(19) United States
(12) Patent Application Publication
(10) Pub. No.: US 2025/0276080 A1
(43) Pub. Date: Sep. 4, 2025

(54) GLUCAGON RECEPTOR AGONISTS AND CONJUGATES TO GLUCOSE-DEPENDENT INSULINOTROPIC POLYPEPTIDE ANTAGONISTS

(71) Applicant: AMGEN INC., Thousand Oaks, CA (US)

(72) Inventors: James R. FALSEY, Moorpark, CA (US); Bin WU, Thousand Oaks, CA (US); Shu-Chen LU, Thousand Oaks, CA (US); Kelvin K.C. SHAM, Thousand Oaks, CA (US); Yuan CHENG, Newbury Park, CA (US); Leslie P. MIRANDA, Thousand Oaks, CA (US); Murielle Marie VENIANT-ELLISON, Thousand Oaks, CA (US); Jennifer ARAL, Newbury Park, CA (US)

(73) Assignee: AMGEN INC., Thousand Oaks, CA (US)

(21) Appl. No.: 19/065,310

(22) Filed: Feb. 27, 2025

Related U.S. Application Data
(60) Provisional application No. 63/559,342, filed on Feb. 29, 2024.

(51) Int. Cl. A61K 47/68; A61P 3/04; C07K 14/605; C07K 16/28

(52) U.S. Cl. CPC A61K 47/6811; A61K 47/6849; A61P 3/04; C07K 14/605; C07K 16/2869; C07K 2317/24; C07K 2317/92

(57) ABSTRACT

Disclosed herein are polypeptides having activity as agonists of a glucagon receptor (“GCGR”), molecules comprising such polypeptides and a half-life extending domain (e.g., an Fc-containing polypeptide), including molecules comprising an antibody that specifically binds to a glucose-dependent insulinotropic polypeptide receptor (“GIPR”), pharmaceutical compositions comprising such polypeptides and molecules, and methods of using such polypeptides, molecules, and pharmaceutical compositions in weight management and the treatment of certain disorders such as obesity.

### CROSS-REFERENCE TO RELATED APPLICATIONS

This application claims the benefit of priority to U.S. Provisional Patent Application No. 63/559,342, filed Feb. 29, 2024, which is hereby incorporated by reference in its entirety.

### SUBMISSION OF SEQUENCE LISTING

The content of the following Sequence Listing XML is incorporated herein by reference in its entirety: file name: 10674-WO01-SEC_Seglisting, date created: Feb. 21, 2025; size: 1,917,591 bytes.

### FIELD

The present disclosure provides polypeptides that agonize a glucagon receptor (“GCGR”), molecules comprising such polypeptides and a half-life extending domain (e.g., an Fc-containing polypeptide), including molecules comprising a polypeptide that agonizes a GCGR and an antibody that specifically binds to a glucose-dependent insulinotropic polypeptide receptor (“GIPR”), pharmaceutical compositions comprising such polypeptides and molecules, and methods of using such polypeptides, molecules, and pharmaceutical compositions in weight management and the treatment of obesity.

### BACKGROUND

Obesity is a chronic, heterogeneous, neurometabolic disease that has grown into an extremely prevalent public health problem. Obesity is projected to affect nearly a quarter of the world's population by 2035 (World Obesity Federation's World Obesity Atlas 2023), and obesity-associated morbidity has exerted a tremendous burden on patients and the healthcare system globally.

Incretin-based therapeutics have transformed type 2 diabetes management, with some recently developed agents, such as the glucagon-like peptide-1 (GLP-1) agonist semaglutide and the GLP-1/glucose-dependent insulinotropic polypeptide (GIP) dual agonist tirzepatide, also promoting notable weight reductions in diabetic and non-diabetic patients. GLP-1 and GIP are endogenous, gut-derived incretin hormones that regulate weight through their receptors. These incretins augment glucose-stimulated insulin secretion and play important roles in weight regulation. For example, GIP promotes fat storage in adipocytes, as well as pancreatic islet j-cell function and glucose-dependent insulin secretion, while GLP-1 promotes satiety.

GIP, formerly known as gastric inhibitory polypeptide, is a single 42-amino acid peptide secreted from K-cells in the small intestine (duodenum and jejunum). Food ingestion induces GIP secretion. Human GIP is derived from the processing of proGIP, a 153-amino acid precursor that is encoded by a gene localized to chromosome 17q. (Inagaki et al., Mol Endocrinol 1989, 3:1014-1021; Fehmann et al. Endocr Rev. 1995; 16:390-410.)

The GIP receptor (GIPR) is a member of the secretin-glucagon family of G-protein coupled receptors (GPCRs). GIPR is expressed in a number of tissues, including the pancreas, gut, adipose tissue, heart, pituitary, adrenal cortex, and brain. (Usdin et al., Endocrinology, 1993, 133:2861-2870.) GIPR knockout mice (Gipr^{-/-}) are resistant to high fat diet-induced weight gain and have improved insulin sensitivity and lipid profiles. (Yamada et al., Diabetes, 2006, 55:S86; Miyawaki et al., Nature Med., 2002, 8:738-742.)

GLP-1 is a 31-amino acid peptide derived from the proglucagon gene. It is secreted by intestinal L-cells and released in response to food ingestion to induce insulin secretion from pancreatic β-cells. (Baggio et al., Diabetes, 2004, 53(S3):S205-S214.) In addition to its incretin effects, GLP-1 also decreases glucagon secretion, delays gastric emptying, and reduces caloric intake. (Drucker, Diabetes Care, 2003, 26(10): 2929-2940.) GLP-1 exerts its effects by activating the GLP-1 receptor, which belongs to a class B G-protein-coupled receptor. GLP-1 is rapidly degraded by the DPP-IV enzyme, resulting in a physiological half-life of approximately two minutes. Recently, long-lasting GLP-1 receptor agonists (GLP-1 RAs) such as exenatide, liraglutide, dulaglutide, and semaglutide have been developed and are now being used clinically to improve glycemic control in patients with type 2 diabetes.

While incretin-based therapeutics have transformed the obesity treatment landscape, not all patients are able to tolerate GLP-1-based therapies, which have been associated with an increased risk of gastrointestinal adverse events (e.g., biliary disease, pancreatitis, bowel obstruction, and gastroparesis) in some patients. (Sodhi et al., JAMA, 2023, 330(18):1795-1797.) Moreover, some patients hit a weight loss plateau on GLP-1-based therapies but still need to further reduce their body weight. Accordingly, there remains a need for alternative therapeutic agents for use in weight management and the treatment or amelioration of obesity.

### SUMMARY

Glucagon is a 29-amino acid peptide hormone (HSQGT FTSDY SKYLD SRRAQ DFVQW LMNT (SEQ ID NO: 1576)) secreted by α-cells of the islet of Langerhans in the pancreas. Glucagon is involved in glucose homeostasis, lipolysis, and amino acid catabolism. Under normal physiological conditions, glucagon levels increase when blood glucose falls, which causes glycogen in the liver to be broken down into glucose for release into the bloodstream. Acute glucagon administration has been associated with increased energy expenditure and circulating insulin and glucose concentrations in adults without diabetes. (Frampton et al., IJO, 2022, 48:1948-1959.)

Provided herein are polypeptides having activity as agonists of a glucagon receptor (“GCGR”) and molecules comprising such polypeptides and a half-life extending domain (e.g., an Fc-containing polypeptide), including molecules comprising an antibody that specifically binds to a glucose-dependent insulinotropic polypeptide receptor (“GIPR”). These polypeptides and molecules, e.g., molecules comprising an anti-GIPR antibody and a GCGR agonist (“GIPR×GCG”), may reduce body weight, liver weight, and fat mass and improve plasma glucose and insulin levels and plasma lipid profiles in obese subjects, alone or in combination with GLP-1 based therapies, e.g., semaglutide. Also provided herein are pharmaceutical compositions comprising these polypeptides and molecules, uses of the polypeptides, molecules, and pharmaceutical compositions in weight management and the treatment of, for example, obesity.

One aspect of the disclosure provides a polypeptide that agonizes a glucagon receptor (“GCGR”), wherein:
the polypeptide comprises at least 25 amino acids, wherein the polypeptide comprises 5-bromo-tryptophan at position 25; and
the polypeptide has at least 79% (e.g., at least 82%, at least 86%, at least 93%, at least 96%, or 100%) sequence identity to the amino acid sequence of SEQ ID NO: 1587.

Another aspect of the disclosure provides polypeptide that agonizes a GCGR, wherein:
the polypeptide comprises at least 28 amino acids, wherein the polypeptide comprises 2-aminoisobutyric acid at position 16 and lysine, serine, or aspartic acid at position 28; and
the polypeptide has at least 89% (e.g., at least 93%, at least 96%, or 100%) sequence identity to the amino acid sequence of SEQ ID NO: 1596.

Still another aspect of the disclosure provides a polypeptide that agonizes a GCGR, wherein:
the polypeptide comprises at least 28 amino acids, wherein the polypeptide comprises 2-aminoisobutyric acid at position 16, leucine at position 27, and lysine at position 28; and
the polypeptide has at least 89% sequence identity to the amino acid sequence of SEQ ID NO: 1615.

Yet another aspect of the disclosure provides a polypeptide that agonizes a GCGR, wherein:
the polypeptide comprises at least 28 amino acids, wherein the polypeptide comprises 2-aminoisobutyric acid at position 16, aspartic acid at position 24, and lysine at position 28; and
the polypeptide has at least 89% (e.g., at least 93%, at least 96%, or 100%) sequence identity to the amino acid sequence of SEQ ID NO: 1626.

In some embodiments, the polypeptide further comprises d-serine at position 2. In some embodiments, the polypeptide further comprises lysine at position 17. In some embodiments, the polypeptide further comprises d-serine at position 2 and lysine at position 17.

Another aspect of the disclosure provides a polypeptide that agonizes a GCGR, wherein:
the polypeptide comprises at least 28 amino acids, wherein the polypeptide comprises a d-serine at position 2 and a 2-aminoisobutyric acid at position 16; and
the polypeptide has at least 89% (e.g., at least 93%, at least 96%, or 100%) sequence identity to the amino acid sequence of SEQ ID NO: 1822.

A further aspect of the disclosure provides a polypeptide comprising an amino acid sequence with between three and nine modifications relative to SEQ ID NO: 1576, wherein the modifications are selected from:
tyrosine and phenylalanine at position 1;
d-serine, 2-aminoisobutyric acid, and d-threonine at position 2;
glutamic acid at position 3;
histidine at position 7;
tryptophan at position 10;
glutamic acid at position 15;
2-aminoisobutyric acid, glutamine, homophenylalanine, and glutamic acid at position 16;
lysine, citrulline, glutamine, and alanine at position 17;
2-naphthylalanine, L-4,4′-biphenylalanine, alanine, citrulline, and lysine at position 18;
4-chloro-L-phenylalanine, alanine, d-glutamine, homoserine, histidine, arginine, and glutamic acid at position 20;
glutamic acid, citrulline, and d-aspartic acid at position 21;
tryptophan and β-cyclohexyl-L-alanine at position 22;
aspartic acid, lysine, alanine, 2-aminoisobutyric acid, glycine, histidine, asparagine, threonine, d-glutamine, glutamic acid, arginine, phenylalanine, leucine, serine, tyrosine, valine, isoleucine, homoserine, and 2,3-diaminopropionic acid at position 24;
5-bromo-L-tryptophan, tyrosine, L-beta-homotryptophan, 5-methoxy-L-tryptophan, 5-methyl-L-tryptophan, 6-bromo-L-tryptophan, 6-chloro-L-tryptophan, 6-methyl-L-tryptophan, and 7-bromo-L-tryptophan at position 25;
leucine, glutamic acid, and L-α-aminoadipic acid at position 27;
lysine, aspartic acid, serine, 6-azido-L-lysine, glutamic acid, and alanine at position 28;
glutamic acid, serine, aspartic acid, and alanine at position 29;
an additional amino acid at position 30, wherein the additional amino acid is lysine; and
an additional amino acid at position 31, wherein the additional amino acid is lysine.

In some embodiments, the polypeptide comprises 31 amino acids. In some embodiments, the polypeptide comprises 31 amino acids, wherein the additional amino acid at position 30 is lysine and the additional amino acid at position 31 is lysine.

In some embodiments, the polypeptide comprises 29 amino acids.

In some embodiments, the modifications comprise a d-serine at position 2, a 2-aminoisobutyric acid at position 16, and between one and seven other modifications at positions 1, 3, 7, 10, 15, 17, 18, 20, 21, 22, 24, 25, 26, 27, 28, 29, 30, or 31 (e.g., at positions 17, 21, 24, 25, 27, 28, or 29) as described above. In some embodiments, the modifications are selected from:
lysine at position 17;
glutamic acid at position 21;
lysine, alanine, and glutamic acid at position 24;
5-bromo-L-tryptophan at position 25;
leucine and glutamic acid at position 27;
lysine, aspartic acid, and glutamic acid at position 28; and
glutamic acid and serine at position 29.

In some embodiments, the polypeptide comprises 29 amino acids.

Another aspect of the disclosure provides a polypeptide that agonizes a GCGR comprising the amino acid sequence of any one of SEQ ID NOs: 1587-1627, 1747-1840, 1859-1862, or 1879-1881. In some embodiments, the polypeptide comprises the amino acid sequence of any one of SEQ ID NOs: 1587-1627 or 1747. In some embodiments, the polypeptide comprises the amino acid sequence of any one of SEQ ID NOs: 1747-1840, 1859-1862, or 1879-1881.

Still another aspect of the disclosure provides a polypeptide that agonizes a GCGR comprising the amino acid sequence of any one of SEQ ID NOs: 1587-1627.

Yet another aspect of the disclosure provides a molecule comprising:
a first polypeptide that agonizes a GCGR, wherein the first polypeptide is selected from those described herein; and
a second polypeptide comprising the amino acid sequence of any one of SEQ ID NOs: 1628-1683 (preferably SEQ ID NO: 1628 or SEQ ID NO: 1629),
wherein the C-terminus of the second polypeptide is covalently linked to an ε-amino group of a lysine residue of the first polypeptide.

In some embodiments, the first polypeptide is glucagon or a glucagon analog. In some embodiments, the first polypeptide is glucagon. In some embodiments, the first polypeptide is human glucagon. In some embodiments, the first polypeptide is a human glucagon analog.

In some embodiments, the first polypeptide comprises an amino acid sequence selected from SEQ ID NOs: 1587-1627, 1747-1749, 1751-1792, 1794-1798, 1801-1830, 1833-1840, and 1859-1862. In some embodiments, the first polypeptide comprises an amino acid sequence selected from SEQ ID NOs: 1587-1627 and 1747.

In some embodiments, the N-terminal amino acid residue of the second polypeptide is modified for coupling to a cysteine residue (e.g., is bromoacetylated).

Another aspect of the disclosure provides a molecule comprising:
a first polypeptide that agonizes a GCGR, wherein the first polypeptide is selected from those described herein; and
a second polypeptide comprising the amino acid sequence of any one of SEQ ID NOs: 1628-1683, 1739, 1850, or 1851,
wherein the C-terminus of the second polypeptide is covalently linked to an ε-amino group of a lysine residue of the first polypeptide.

In some embodiments, the first polypeptide comprises an amino acid sequence selected from SEQ ID NOs: 1587-1627, 1747-1749, 1751-1792, 1794-1798, 1801-1830, 1833-1840, and 1859-1862. In some embodiments, the second polypeptide comprises the amino acid sequence of any one of SEQ ID NOs: 1628, 1629, 1630, 1631, 1632, 1640, or 1644. In some embodiments, the first polypeptide comprises an amino acid sequence selected from SEQ ID NOs: 1587-1627, 1747-1749, 1751-1792, 1794-1798, 1801-1830, 1833-1840, and 1859-1862, and the second polypeptide comprises an amino acid sequence selected from SEQ ID NOs: 1628, 1629, 1630, 1631, 1632, 1640, or 1644.

In some embodiments, the C-terminal amino acid residue of the first polypeptide is modified (e.g., amidated).

In some embodiments, the N-terminal amino acid residue of the second polypeptide is modified for coupling to a cysteine residue (e.g., is bromoacetylated).

Yet another aspect of the disclosure provides a molecule comprising
a first polypeptide that agonizes a GCGR, wherein the first polypeptide is selected from those described herein; and
a second polypeptide comprising the amino acid sequence of any one of SEQ ID NOs: 1740-1746, 1841-1849, or 1852,
wherein the C-terminus/C-terminal amino acid residue of the first polypeptide is covalently linked to the N-terminus/N-terminal amino acid residue of the second polypeptide.

In some embodiments, the first polypeptide is glucagon or a glucagon analog. In some embodiments, the first polypeptide is glucagon. In some embodiments, the first polypeptide is human glucagon. In some embodiments, the first polypeptide is a glucagon analog. In some embodiments, the first polypeptide is a human glucagon analog.

In some embodiments, the first polypeptide comprises an amino acid sequence selected from SEQ ID NOs: 1793, 1799, 1800, 1831, and 1832.

In some embodiments, the C-terminal amino acid residue of the second polypeptide is a lysine residue. In some embodiments, the C-terminal amino acid residue of the second polypeptide is modified for coupling to a cysteine residue (e.g., is bromoacetylated). In some embodiments, the C-terminal amino acid residue of the second polypeptide is a bromoacetylated lysine residue.

Still another aspect of the disclosure provides a molecule comprising a polypeptide that agonizes a GCGR, wherein the polypeptide is selected from those described herein; and an antigen-binding protein. In some embodiments, the polypeptide comprises an amino acid sequence selected from SEQ ID NOs: 1587-1627, 1747-1840, and 1859-1862. In some embodiments, the polypeptide comprises an amino acid sequence selected from SEQ ID NOs: 1587-1627 and 1747. In some embodiments, the polypeptide comprises an amino acid sequence selected from SEQ ID NOs: 1747-1840 and 1859-1862.

Yet another aspect of the disclosure provides a molecule comprising: a polypeptide that agonizes a GCGR, wherein the polypeptide is selected from those described herein; and a half-life extending domain (e.g., an Fc-containing polypeptide, such as, e.g., an antibody). In some embodiments, the polypeptide comprises an amino acid sequence selected from SEQ ID NOs: 1587-1627, 1747-1840, and 1859-1862. In some embodiments, the polypeptide comprises an amino acid sequence selected from SEQ ID NOs: 1587-1627 and 1747. In some embodiments, the polypeptide comprises an amino acid sequence selected from SEQ ID NOs: 1747-1840 and 1859-1862.

Still another aspect of the disclosure provides a molecule comprising a polypeptide that agonizes a GCGR, wherein the polypeptide is selected from those described herein; and an antibody that specifically binds to a glucose-dependent insulinotropic polypeptide receptor (“GIPR”).

In some embodiments, the polypeptide is glucagon or a glucagon analog. In some embodiments, the polypeptide is glucagon. In some embodiments, the polypeptide is human glucagon. In some embodiments, the polypeptide is a glucagon analog. In some embodiments, the polypeptide is a human glucagon analog.

In some embodiments, the polypeptide comprises an amino acid sequence selected from SEQ ID NOs: 1587-1627, 1747-1840, and 1859-1862. In some embodiments, the polypeptide comprises an amino acid sequence selected from SEQ ID NOs: 1587-1627 and 1747. In some embodiments, the polypeptide comprises an amino acid sequence selected from SEQ ID NOs: 1747-1840 and 1859-1862.

Another aspect of the disclosure provides a molecule comprising: a polypeptide that agonizes a glucagon receptor (“GCGR”); a linker polypeptide; and an antibody that specifically binds to a glucose-dependent insulinotropic polypeptide receptor (“GIPR”), wherein:
a lysine residue or an azido-lysine residue of the polypeptide is covalently linked to a C-terminus of the linker polypeptide; and
an N-terminus of the linker polypeptide is conjugated to a cysteine residue of the antibody.

In some embodiments, the polypeptide is glucagon or a glucagon analog. In some embodiments, the polypeptide is glucagon. In some embodiments, the polypeptide is human glucagon. In some embodiments, the polypeptide is a glucagon analog. In some embodiments, the polypeptide is a human glucagon analog.

In some embodiments, the polypeptide comprises an amino acid sequence selected from SEQ ID NOs: 1587-1627, 1747-1840, and 1859-1862. In some embodiments, the polypeptide comprises an amino acid sequence selected from SEQ ID NOs: 1587-1627 and 1747. In some embodiments, the polypeptide comprises an amino acid sequence selected from SEQ ID NOs: 1747-1840 and 1859-1862.

In some embodiments, an ε-amino group of a lysine residue at position 21, position 24, position 28, or position 31 (e.g., at position 24 or position 28) of the polypeptide is covalently linked to a C-terminus of the linker polypeptide. In some embodiments, an azido-lysine residue at position 28 of the polypeptide is covalently linked to a C-terminus of the linker polypeptide.

In some embodiments, the C-terminal amino acid residue of the polypeptide is modified (e.g., amidated).

Yet another aspect of the disclosure provides a molecule comprising: a polypeptide that agonizes a glucagon receptor (“GCGR”); a linker polypeptide; and an antibody that specifically binds to a glucose-dependent insulinotropic polypeptide receptor (“GIPR”),
wherein:
   an ε-amino group of a lysine residue of the polypeptide is covalently linked to a C-terminus of the linker polypeptide; and
an N-terminus of the linker polypeptide is conjugated to a cysteine residue of the antibody.

In some embodiments, the polypeptide is glucagon or a glucagon analog. In some embodiments, the polypeptide is glucagon. In some embodiments, the polypeptide is human glucagon. In some embodiments, the polypeptide is a glucagon analog. In some embodiments, the polypeptide is a human glucagon analog.

In some embodiments, the polypeptide comprises an amino acid sequence selected from SEQ ID NOs: 1587-1627, 1747-1840, and 1859-1862. In some embodiments, the polypeptide comprises an amino acid sequence selected from SEQ ID NOs: 1587-1627 and 1747. In some embodiments, the polypeptide comprises an amino acid sequence selected from SEQ ID NOs: 1747-1840 and 1859-1862.

In some embodiments, an ε-amino group of a lysine residue at position 21, position 24, position 28, or position 31 of the polypeptide is covalently linked to a C-terminus of the linker polypeptide.

Still another aspect of the disclosure provides a molecule comprising: a polypeptide that agonizes a glucagon receptor (“GCGR”); a linker polypeptide; and an antibody that specifically binds to a glucose-dependent insulinotropic polypeptide receptor (“GIPR”), wherein:
a C-terminus/C-terminal amino acid residue of the polypeptide is covalently linked to an N-terminus/N-terminal amino acid residue of the linker polypeptide; and
a C-terminus/C-terminal amino acid residue of the linker polypeptide is conjugated to a cysteine residue of the antibody.

In some embodiments, the C-terminal amino acid residue of the linker polypeptide is a lysine residue.

In some embodiments, the polypeptide is glucagon or a glucagon analog. In some embodiments, the polypeptide is glucagon. In some embodiments, the polypeptide is human glucagon. In some embodiments, the polypeptide is a glucagon analog. In some embodiments, the polypeptide is a human glucagon analog.

In some embodiments, the polypeptide comprises an amino acid sequence selected from SEQ ID NOs: 1587-1627, 1747-1840, and 1859-1862. In some embodiments, the polypeptide comprises an amino acid sequence selected from SEQ ID NOs: 1587-1627 and 1747. In some embodiments, the polypeptide comprises an amino acid sequence selected from SEQ ID NOs: 1747-1840 and 1859-1862.

Another aspect of the disclosure provides a molecule comprising:
a polypeptide that agonizes a glucagon receptor (“GCGR”) comprising the amino acid sequence of any one of SEQ ID NOs: 1587-1627, 1747-1749, 1751-1792, 1794-1798, 1801-1830, 1833-1840, 1859-1862, or 1879-1881;
a linker polypeptide comprising the amino acid sequence of any one of SEQ ID NOs: 1628-1683, 1739, 1850, or 1851, preferably SEQ ID NOs: 1628-1630, preferably SEQ ID NO: 1628 or SEQ ID NO: 1629; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 21, position 24, position 28, or position 31 of the polypeptide is covalently linked to a C-terminus of the linker polypeptide; and
   an N-terminus of the linker polypeptide is conjugated to a cysteine residue at position 275 of the heavy chain of the antibody.

In some embodiments, an ε-amino group of a lysine residue at position 24 or position 28 of the polypeptide is covalently linked to a C-terminus of the linker polypeptide.

Still another aspect of the disclosure provides a molecule comprising:
a polypeptide that agonizes a glucagon receptor (“GCGR”) comprising the amino acid sequence of any one of SEQ ID NOs: 1589, 1592, 1594, 1597, 1598, 1613, 1625, 1762, 1766-1768, 1787, 1788, 1797, 1798, 1804, 1805, 1811, 1812, 1821, 1822, 1833, 1837, or 1838;
a linker polypeptide comprising the amino acid sequence of any one of SEQ ID NOs: 1628-1683, 1739, 1850, or 1851; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 24 of the polypeptide is covalently linked to a C-terminus of the linker polypeptide; and
   an N-terminus of the linker polypeptide is conjugated to a cysteine residue at position 275 of the heavy chain of the antibody.

In some embodiments, the linker polypeptide comprises the amino acid sequence of any one of SEQ ID NOs: 1628-1630, 1632, 1634, 1641, 1642, 1644, or 1647.

Yet another aspect of the disclosure provides a molecule comprising:
a polypeptide that agonizes a glucagon receptor (“GCGR”) comprising the amino acid sequence of any one of SEQ ID NOs: 1587, 1588, 1590, 1591, 1593, 1595, 1596, 1599-1612, 1614-1624, 1626, 1627, 1747-1749, 1751-1761, 1763-1765, 1769-1786, 1790-1792, 1794-1796, 1801-1803, 1806-1810, 1813-1820, 1823-1830, 1834-1836, 1839, 1840, 1859, 1861, or 1862;
a linker polypeptide comprising the amino acid sequence of any one of SEQ ID NOs: 1628-1683, 1739, 1850, or 1851; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

In some embodiments, the linker polypeptide comprises the amino acid sequence of any one of SEQ ID NOs: 1628-1632, 1635, 1638-1640, 1642, 1644, 1647, 1850, or 1851. In some embodiments, the linker polypeptide comprises the amino acid sequence of any one of SEQ ID NOs: 1628-1632, 1635, 1638-1640, 1642, 1644, or 1647.

Still another aspect of the disclosure provides a molecule comprising:
a polypeptide that agonizes a glucagon receptor (“GCGR”) comprising the amino acid sequence of any one of SEQ ID NOs: 1587-1627 (e.g., SEQ ID NOs: 1587, 1592, 1596, 1615, or 1626);
a linker polypeptide comprising the amino acid sequence of any one of SEQ ID NOs: 1628-1683 (e.g., SEQ ID NOs: 1628-1630); and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 24 or position 28 of the polypeptide is covalently linked to a C-terminus of the linker polypeptide; and
   an N-terminus of the linker polypeptide is conjugated to a cysteine residue at position 275 of the heavy chain of the antibody.

Another aspect of the disclosure provides a molecule comprising: a first polypeptide that agonizes a glucagon receptor (“GCGR”); a first linker polypeptide; an antibody that specifically binds to a glucose-dependent insulinotropic polypeptide receptor (“GIPR”); a second linker polypeptide; and a second polypeptide that agonizes a GCGR, wherein:
a C-terminus/C-terminal amino acid residue of the first polypeptide is covalently linked to an N-terminus/N-terminal amino acid residue of the first linker polypeptide;
a C-terminus/C-terminal amino acid residue of the first linker polypeptide is conjugated to a cysteine residue of the antibody;
a C-terminus/C-terminal amino acid residue of the second polypeptide is covalently linked to an N-terminus/N-terminal amino acid residue of the second linker polypeptide; and
a C-terminus/C-terminal amino acid residue of the second linker polypeptide is conjugated to a cysteine residue of the antibody.

Yet another aspect of the disclosure provides a molecule comprising: a first polypeptide that agonizes a glucagon receptor (“GCGR”); a first linker polypeptide; an antibody that specifically binds to a glucose-dependent insulinotropic polypeptide receptor (“GIPR”); a second linker polypeptide; and a second polypeptide that agonizes a GCGR,
wherein:
   an ε-amino group of a lysine residue of the first polypeptide is covalently linked to a C-terminus of the first linker polypeptide;
an N-terminus of the first linker polypeptide is conjugated to a cysteine residue of the antibody;
an ε-amino group of a lysine residue of the second polypeptide is covalently linked to a C-terminus of the second linker polypeptide; and
an N-terminus of the second linker polypeptide is conjugated to a cysteine residue of the antibody.

Still another aspect of the disclosure provides a molecule comprising:
a polypeptide that agonizes a glucagon receptor (“GCGR”) comprising the amino acid sequence of SEQ ID NO: 1615;
a linker polypeptide comprising the amino acid sequence of SEQ ID NO: 1629; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Yet another aspect of the disclosure provides a molecule comprising:
a polypeptide that agonizes a glucagon receptor (“GCGR”) comprising the amino acid sequence of SEQ ID NO: 1626;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Still another aspect of the disclosure provides a molecule comprising:
a polypeptide that agonizes a glucagon receptor (“GCGR”) comprising the amino acid sequence of SEQ ID NO: 1592;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 24 of the polypeptide is covalently linked to a C-terminus of the linker polypeptide; and
   an N-terminus of the linker polypeptide is conjugated to a cysteine residue at position 275 of the heavy chain of the antibody.

Yet another aspect of the disclosure provides a molecule comprising:
a polypeptide that agonizes a glucagon receptor (“GCGR”) comprising the amino acid sequence of SEQ ID NO: 1626;
a linker polypeptide comprising the amino acid sequence of SEQ ID NO: 1629; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Still another aspect of the disclosure provides a molecule comprising:
a polypeptide that agonizes a glucagon receptor (“GCGR”) comprising the amino acid sequence of SEQ ID NO: 1587;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Yet another aspect of the disclosure provides a molecule comprising:
a polypeptide that agonizes a glucagon receptor (“GCGR”) comprising the amino acid sequence of SEQ ID NO: 1587;
a linker polypeptide comprising the amino acid sequence of SEQ ID NO: 1630; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Another aspect of the disclosure provides a molecule comprising:
a polypeptide that agonizes a glucagon receptor (“GCGR”) comprising the amino acid sequence of SEQ ID NO: 1822;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 24 of the polypeptide is covalently linked to a C-terminus of the linker polypeptide; and
   an N-terminus of the linker polypeptide is conjugated to a cysteine residue at position 275 of the heavy chain of the antibody.

Still another aspect of the disclosure provides a molecule comprising:
a polypeptide that agonizes a glucagon receptor (“GCGR”) comprising the amino acid sequence of SEQ ID NO: 1825;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Yet another aspect of the disclosure provides a molecule comprising:
a polypeptide that agonizes a glucagon receptor (“GCGR”) comprising the amino acid sequence of SEQ ID NO: 1826;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Still another aspect of the disclosure provides a molecule comprising:
a polypeptide that agonizes a glucagon receptor (“GCGR”) comprising the amino acid sequence of SEQ ID NO: 1818;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Another aspect of the disclosure provides a molecule comprising:
a first polypeptide and a second polypeptide that agonize a glucagon receptor (“GCGR”), wherein each of the first polypeptide and the second polypeptide comprises an amino acid sequence independently selected from SEQ ID NOs: 1587-1627, 1747-1749, 1751-1792, 1794-1798, 1801-1830, 1833-1840, and 1859-1862;
a first linker polypeptide and a second linker polypeptide, wherein each of the first linker polypeptide and the second linker polypeptide comprises an amino acid sequence selected from SEQ ID NOs: 1628-1683, 1739-1746, and 1841-1852; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 21, position 24, position 28, or position 31 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 21, position 24, position 28, or position 31 of the second polypeptide is covalently linked to a C-terminus of the second linker polypeptide; and
   an N-terminus of the second linker polypeptide is conjugated to a cysteine residue at position 275 of the second heavy chain of the antibody.

In some embodiments, the first polypeptide and the second polypeptide each comprise an amino acid sequence independently selected from SEQ ID NOs: 1589, 1592, 1594, 1597, 1598, 1613, 1625, 1762, 1766-1768, 1787, 1788, 1797, 1798, 1804, 1805, 1811, 1812, 1821, 1822, 1833, 1837, and 1838, the ε-amino group of the lysine residue at position 24 of the first polypeptide is covalently linked to the C-terminus of the first linker polypeptide, and the ε-amino group of the lysine residue at position 24 of the second polypeptide is covalently linked to the C-terminus of the second linker polypeptide. In some embodiments, the first polypeptide and the second polypeptide have identical amino acid sequences. In some embodiments, the first linker polypeptide and the second linker polypeptide have identical amino acid sequences. In some embodiments, the first polypeptide and the second polypeptide have identical amino acid sequences, and the first linker polypeptide and the second linker polypeptide have identical amino acid sequences.

In some embodiments, the first polypeptide and the second polypeptide comprise an amino acid sequence independently selected from SEQ ID NOs: 1587, 1588, 1590, 1591, 1593, 1595, 1596, 1599-1612, 1614-1624, 1626, 1627, 1747-1749, 1751-1761, 1763-1765, 1769-1786, 1790-1792, 1794-1796, 1801-1803, 1806-1810, 1813-1820, 1823-1830, 1834-1836, 1839, 1840, 1859, 1861, and 1862, the ε-amino group of the lysine residue at position 28 of the first polypeptide is covalently linked to the C-terminus of the first linker polypeptide, and the ε-amino group of the lysine residue at position 28 of the second polypeptide is covalently linked to the C-terminus of the second linker polypeptide. In some embodiments, the first polypeptide and the second polypeptide have identical amino acid sequences. In some embodiments, the first linker polypeptide and the second linker polypeptide have identical amino acid sequences. In some embodiments, the first polypeptide and the second polypeptide have identical amino acid sequences, and the first linker polypeptide and the second linker polypeptide have identical amino acid sequences.

In some embodiments, the first polypeptide and the second polypeptide each comprise an amino acid sequence of SEQ ID NO: 1860, the ε-amino group of the lysine residue at position 21 of the first polypeptide is covalently linked to the C-terminus of the first linker polypeptide, and the ε-amino group of the lysine residue at position 21 of the second polypeptide is covalently linked to the C-terminus of the second linker polypeptide. In some embodiments, the first linker polypeptide and the second linker polypeptide have identical amino acid sequences.

In some embodiments, the first polypeptide and the second polypeptide each comprise an amino acid sequence of SEQ ID NO: 1789, the ε-amino group of the lysine residue at position 31 of the first polypeptide is covalently linked to the C-terminus of the first linker polypeptide, and the ε-amino group of the lysine residue at position 31 of the second polypeptide is covalently linked to the C-terminus of the second linker polypeptide. In some embodiments, the first linker polypeptide and the second linker polypeptide have identical amino acid sequences.

Still another aspect of the disclosure provides a molecule comprising:
a first polypeptide and a second polypeptide that agonize a glucagon receptor (“GCGR”), wherein each of the first polypeptide and the second polypeptide comprise an amino acid sequence independently selected from SEQ ID NOs: 1587-1627 (e.g., SEQ ID NOs: 1587, 1592, 1596, 1615, or 1626);
a first linker polypeptide and a second linker polypeptide, wherein each of the first linker polypeptide and the second linker polypeptide comprise an amino acid sequence selected from SEQ ID NOs: 1628-1683 (e.g., SEQ ID NOs: 1628-1630); and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 24 or position 28 of the first polypeptide is covalently linked to a C-terminus of the first linker polypeptide; an N-terminus of the first linker polypeptide is conjugated to a cysteine residue at position 275 of the first heavy chain of the antibody;
   an ε-amino group of a lysine residue at position 24 or position 28 of the second polypeptide is covalently linked to a C-terminus of the second linker polypeptide; and
   an N-terminus of the second linker polypeptide is conjugated to a cysteine residue at position 275 of the second heavy chain of the antibody.

In some embodiments, the first polypeptide and the second polypeptide have identical amino acid sequences. In some embodiments, the first linker polypeptide and the second linker polypeptide have identical amino acid sequences. In some embodiments, the first polypeptide and the second polypeptide have identical amino acid sequences, and the first linker polypeptide and the second linker polypeptide have identical amino acid sequences.

Another aspect of the disclosure provides a molecule comprising:
a first polypeptide and a second polypeptide that agonize a glucagon receptor (“GCGR”), wherein each of the first polypeptide and the second polypeptide comprise an amino acid sequence independently selected from SEQ ID NOs: 1587-1627, 1747-1840, and 1859-1862;
a first linker polypeptide and a second linker polypeptide, wherein each of the first linker polypeptide and the second linker polypeptide comprise an amino acid sequence selected from SEQ ID NOs: 1628-1683, 1739-1746, and 1841-1852; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   a C-terminus/C-terminal amino acid residue of the first polypeptide is covalently linked to an N-terminus/N-terminal amino acid residue of the first linker polypeptide;
   a C-terminus/C-terminal amino acid residue of the first linker polypeptide is conjugated to a cysteine residue at position 275 of the first heavy chain of the antibody;
   a C-terminus/C-terminal amino acid residue of the second polypeptide is covalently linked to an N-terminus/N-terminal amino acid residue of the second linker polypeptide; and
   a C-terminus/C-terminal amino acid residue of the second linker polypeptide is conjugated to a cysteine residue at position 275 of the second heavy chain of the antibody.

In some embodiments, each of the first polypeptide and the second polypeptide comprise an amino acid sequence independently selected from SEQ ID NOs: 1793, 1799, 1800, 1831, and 1832. In some embodiments, each of the first linker polypeptide and the second linker polypeptide comprise an amino acid sequence independently selected from SEQ ID NOs: 1740-1746, 1841-1849, or 1852. In some embodiments, each of the first polypeptide and the second polypeptide comprise an amino acid sequence independently selected from SEQ ID NOs: 1793, 1799, 1800, 1831, and 1832, and each of the first linker polypeptide and the second linker polypeptide comprise an amino acid sequence independently selected from SEQ ID NOs: 1740-1746, 1841-1849, or 1852.

In some embodiments, the first polypeptide and the second polypeptide have identical amino acid sequences. In some embodiments, the first linker polypeptide and the second linker polypeptide have identical amino acid sequences. In some embodiments, the first polypeptide and the second polypeptide have identical amino acid sequences, and the first linker polypeptide and the second linker polypeptide have identical amino acid sequences.

Yet another aspect of the disclosure provides a molecule comprising:
a first polypeptide and a second polypeptide that agonize a glucagon receptor (“GCGR”), wherein the first polypeptide and the second polypeptide each comprise the amino acid sequence of SEQ ID NO: 1615;
a first linker polypeptide and a second linker polypeptide, wherein the first linker polypeptide and the second linker polypeptide each comprise the amino acid sequence of SEQ ID NO: 1629; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide; an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Still another aspect of the disclosure provides a molecule comprising: a first polypeptide and a second polypeptide that agonize a glucagon receptor (“GCGR”), wherein the first polypeptide and the second polypeptide each comprise the amino acid sequence of SEQ ID NO: 1626;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Yet another aspect of the disclosure provides a molecule comprising: a first polypeptide and a second polypeptide that agonize a glucagon receptor (“GCGR”), wherein the first polypeptide and the second polypeptide each comprise the amino acid sequence of SEQ ID NO: 1592;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 24 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 24 of the second polypeptide is covalently linked to a C-terminus of the second linker polypeptide; and
   an N-terminus of the second linker polypeptide is conjugated to a cysteine residue at position 275 of the second heavy chain of the antibody.

Still another aspect of the disclosure provides a molecule comprising:
a first polypeptide and a second polypeptide that agonize a glucagon receptor (“GCGR”), wherein the first polypeptide and the second polypeptide each comprise the amino acid sequence of SEQ ID NO: 1626;
a first linker polypeptide and a second linker polypeptide, wherein the first linker polypeptide and the second linker polypeptide each comprise the amino acid sequence of SEQ ID NO: 1629; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide; an N-terminus of the first linker polypeptide is conjugated to a cysteine residue at position 275 of the first heavy chain of the antibody;
an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Yet another aspect of the disclosure provides a molecule comprising:
a first polypeptide and a second polypeptide that agonize a glucagon receptor (“GCGR”), wherein the first polypeptide and the second polypeptide each comprise the amino acid sequence of SEQ ID NO: 1587;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide; an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Still another aspect of the disclosure provides a molecule comprising:
a first polypeptide and a second polypeptide that agonize a glucagon receptor (“GCGR”), wherein the first polypeptide and the second polypeptide each comprise the amino acid sequence of SEQ ID NO: 1587;
a first linker polypeptide and a second linker polypeptide, wherein the first linker polypeptide and the second linker polypeptide each comprise the amino acid sequence of SEQ ID NO: 1630; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Another aspect of the disclosure provides a molecule comprising:
a first polypeptide and a second polypeptide that agonize a glucagon receptor (“GCGR”), wherein the first polypeptide and the second polypeptide each comprise the amino acid sequence of SEQ ID NO: 1822;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 24 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 24 of the second polypeptide is covalently linked to a C-terminus of the second linker polypeptide; and
   an N-terminus of the second linker polypeptide is conjugated to a cysteine residue at position 275 of the second heavy chain of the antibody.

Still another aspect of the disclosure provides a molecule comprising:
a first polypeptide and a second polypeptide that agonize a glucagon receptor (“GCGR”), wherein the first polypeptide and the second polypeptide each comprise the amino acid sequence of SEQ ID NO: 1825;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Yet another aspect of the disclosure provides a molecule comprising:
a first polypeptide and a second polypeptide that agonize a glucagon receptor (“GCGR”), wherein the first polypeptide and the second polypeptide each comprise the amino acid sequence of SEQ ID NO: 1826;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Another aspect of the disclosure provides a molecule comprising:
a first polypeptide and a second polypeptide that agonize a glucagon receptor (“GCGR”), wherein the first polypeptide and the second polypeptide each comprise the amino acid sequence of SEQ ID NO: 1818;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Yet another aspect of the disclosure provides a pharmaceutical composition comprising a polypeptide or molecule disclosed herein and a pharmaceutically acceptable excipient.

Still another aspect of the disclosure provides a method of treating obesity in a subject in need of treatment, the method comprising administering a polypeptide, molecule, or a pharmaceutical composition to the subject.

Another aspect of the disclosure provides a method of reducing body weight and/or food intake in a subject in need thereof (e.g., an overweight or obese subject), the method comprising administering a polypeptide, molecule, or a pharmaceutical composition to the subject.

Yet another aspect of the disclosure provides a polypeptide or molecule disclosed herein for use as a medicament. Another aspect of the disclosure provides a polypeptide or molecule disclosed herein, or a pharmaceutical composition disclosed herein, for use in the treatment of obesity. Still another aspect of the disclosure provides a polypeptide or molecule disclosed herein, or a pharmaceutical composition disclosed herein, for use in a method of reducing body weight and/or food intake in a subject in need thereof (e.g., an overweight or obese subject).

Yet another aspect of the disclosure provides a polypeptide or molecule disclosed herein for the manufacture of a medicament for the treatment of obesity. Another aspect of the disclosure provides a polypeptide or molecule disclosed herein for the manufacture of a medicament for reducing body weight and/or food intake in a subject in need thereof (e.g., an overweight or obese subject).

Another aspect of the disclosure provides a GCGR agonist and a GIPR antagonist for use in therapy. Yet another aspect of the disclosure provides a GCGR agonist for use in therapy in combination with a GIPR antagonist. Still another aspect provides a GIPR antagonist for use in therapy in combination with a GCGR agonist. In some embodiments, the therapy is for use in weight management. In some embodiments, the therapy is for use in treating obesity. In some cases, the GCGR agonist and the GIPR antagonist may be used in acute therapy. In other cases, the GCGR agonist and the GIPR antagonist may be used in chronic therapy. The GCGR agonist and the GIPR antagonist may be present in the same or separate pharmaceutical compositions.

Another aspect of the disclosure provides a use of a GCGR agonist and a GIPR antagonist in the preparation of a medicament. Yet another aspect of the disclosure provides a use of a GCGR agonist in the preparation of a medicament for use in combination therapy with a GIPR antagonist. Still another aspect of the disclosure provides a use of a GIPR antagonist in the preparation of a medicament for use in combination therapy with a GCGR agonist. In some embodiments, the medicament is for use in combination therapy for weight management. In some embodiments, the medicament is for use in combination therapy in treating obesity.

Another aspect of the disclosure provides a method of treating obesity in a subject in need thereof, the method comprising administering a glucagon receptor (GCGR) agonist and a GIPR antagonist to the subject. Still another aspect of the disclosure provides a method of reducing body weight and/or food intake in a subject in need thereof, the method comprising administering a glucagon receptor (GCGR) agonist and a GIPR antagonist to the subject.

Another aspect of the disclosure provides a GCGR agonist and a GIPR antagonist for use in treating obesity. Yet another aspect of the disclosure provides a GCGR agonist for use in treating obesity in combination with a GIPR antagonist. Still another aspect of the disclosure provides a GIPR antagonist for use in treating obesity in combination with a GCGR agonist.

Another aspect of the disclosure provides a GCGR agonist and a GIPR antagonist for use in reducing body weight and/or food intake in a subject in need thereof. Yet another aspect of the disclosure provides a GCGR agonist for use in reducing body weight and/or food intake in a subject in need thereof in combination with a GIPR antagonist. Still another aspect of the disclosure provides a GIPR antagonist for use in reducing body weight and/or food intake in a subject in need thereof in combination with a GCGR agonist.

Another aspect of the disclosure provides a use of a GCGR agonist and a GIPR antagonist in the manufacture of a medicament for treating obesity. Yet another aspect of the disclosure provides a use of a GCGR agonist in the manufacture of a medicament for treating obesity in combination with a GIPR antagonist. Still another aspect of the disclosure provides a use of a GIPR antagonist in the manufacture of a medicament for treating obesity in combination with a GCGR agonist.

Another aspect of the disclosure provides a use of a GCGR agonist and a GIPR antagonist in the manufacture of a medicament for reducing body weight and/or food intake in a subject in need thereof. Yet another aspect of the disclosure provides a use of a GCGR agonist in the manufacture of a medicament for reducing body weight and/or food intake in a subject in need thereof in combination with a GIPR antagonist. Still another aspect of the disclosure provides a use of a GIPR antagonist in the manufacture of a medicament for reducing body weight and/or food intake in a subject in need thereof in combination with a GCGR agonist.

Another aspect of the disclosure provides a combination therapy comprising a GLP-1 agonist (e.g., semaglutide) and a polypeptide or molecule disclosed herein for use in weight management. Still another aspect of the disclosure provides a combination therapy comprising a GLP-1 agonist (e.g., semaglutide) and a polypeptide or molecule disclosed herein for use in the treatment of obesity. Another aspect of the disclosure provides a combination therapy comprising a GLP-1 agonist (e.g., semaglutide) and a polypeptide or molecule disclosed herein for use in a method of reducing body weight and/or food intake in a subject in need thereof (e.g., an overweight or obese subject).

Further aspects and advantages will be apparent to those of ordinary skill in the art from a review of the following detailed description. The description hereafter includes specific cases, embodiments, and examples with the understanding that the disclosure is illustrative and is not intended to limit the embodiments of the present disclosure to the specific cases, embodiments, and examples described herein.

### BRIEF DESCRIPTION OF THE DRAWINGS

FIG. 1A depicts cAMP levels expressed as a fluorescence ratio of 665/620 nm in CHOK1 cells stably expressing human GCGR following exposure to six example GIPR×GCG conjugates and a positive control.

FIG. 1B depicts cAMP levels expressed as a fluorescence ratio of 665/620 nm in primary human hepatocytes expressing human GCGR following exposure to an example GIPR×GCG conjugate and a positive control.

FIG. 1C depicts cAMP levels expressed as a fluorescence ratio of 665/620 nm in primary mouse hepatocytes expressing mouse GCGR following exposure to an example GIPR×GCG conjugate and a positive control.

FIG. 2 shows cAMP levels expressed as a fluorescence ratio of 665/620 nm in CHOK1 cells stably expressing human GLP-1R following exposure to six example GIPR×GCG conjugates and a positive control.

FIG. 3A provides cAMP levels expressed as a fluorescence ratio of 665/620 nm in HEK 293T cells stably expressing human GIPR following exposure to six example GIPR×GCG conjugates and a positive control.

FIG. 3B provides cAMP levels expressed as a fluorescence ratio of 665/620 nm in CHO AMID cells expressing mouse GIPR following exposure to an example GIPR×GCG conjugate and a positive control.

FIGS. 4A-4D depict changes in body weight from baseline for mice with diet-induced obesity treated with either vehicle, a long-lasting GCG agonist (DNP×GCG conjugate), or a GIPR×GCG conjugate.

FIG. 5A shows changes in body weight from baseline for mice with diet-induced obesity treated with either vehicle, a long-lasting GCG agonist (DNP×GCG conjugate), or a GIPR×GCG conjugate.

FIGS. 5B and 5C show blood glucose levels 3 hours (FIG. 5B) or 24 and 72 hours (FIG. 5C), respectively, post-injection of vehicle, a long-lasting GCG agonist (DNP×GCG conjugate), or a GIPR×GCG conjugate in diet-induced obese mice.

FIG. 6A depicts changes in body weight from baseline for mice with diet-induced obesity treated with vehicle or one of six example GIPR×GCG conjugates.

FIG. 6B shows liver triglycerides in milligrams of triacylglycerol (TGA) per gram of liver tissue on day 14 of a study in which mice with diet-induced obesity were treated with vehicle or one of six example GIPR×GCG conjugates.

FIGS. 6C-6E provide liver tissue (FIG. 6C), inguinal white adipose tissue (FIG. 6D), and epididymal white adipose tissue (FIG. 6E) weights on day 14 of a study in which mice with diet-induced obesity were treated with vehicle or one of six example GIPR×GCG conjugates.

FIGS. 6F-6J depict plasma glucose (FIG. 6F), plasma insulin (FIG. 6G), plasma cholesterol (FIG. 6H), plasma low-density lipoprotein (LDL)-cholesterol (C) (FIG. 6I), and plasma triglyceride (FIG. 6J) levels on day 14 of a study in which mice with diet-induced obesity were treated with vehicle or one of six example GIPR×GCG conjugates.

FIGS. 7A and 7B show blood glucose levels over 90 minutes (FIG. 7A) and glucose area under the curve (AUC) (FIG. 7B) observed during an oral glucose tolerance test (OGTT) in diet-induced obese (DIO) mice pre-treated with vehicle or one of five example GIPR×GCG conjugates.

FIGS. 7C and 7D show plasma insulin levels over 90 minutes (FIG. 7C) and insulin area under the curve (AUC) (FIG. 7D) observed during OGTT in DIO mice pre-treated with vehicle or one of five example GIPR×GCG conjugates.

FIG. 8A depicts changes in body weight from baseline for DIO mice treated with vehicle, the GLP-1 agonist semaglutide, semaglutide followed by a GIPR×GCG conjugate, or semaglutide plus a GIPR×GCG conjugate over a 28-day study period.

FIG. 8B depicts daily food intake for DIO mice treated with vehicle, the GLP-1 agonist semaglutide, semaglutide followed by a GIPR×GCG conjugate, or semaglutide plus a GIPR×GCG conjugate over a 28-day study period.

FIGS. 8C-8G provide inguinal white adipose tissue (FIG. 8C), epididymal white adipose tissue (FIG. 8D), liver tissue (FIG. 8E), kidney tissue (paired) (FIG. 8F), and brain tissue (FIG. 8G) weights on day 28 of a study in which mice with diet-induced obesity were treated with vehicle, semaglutide, semaglutide followed by a GIPR×GCG conjugate, or semaglutide plus a GIPR×GCG conjugate.

FIGS. 8H-8M show plasma triglyceride (FIG. 8H), plasma cholesterol (FIG. 8I), plasma high-density lipoprotein (HDL)-cholesterol (C) (FIG. 8J), plasma LDL-C (FIG. 8K), plasma glucose (FIG. 8L), and plasma insulin levels (FIG. 8M) on day 28 of a study in which mice with diet-induced obesity were treated with vehicle, semaglutide, semaglutide followed by a GIPR×GCG conjugate, or semaglutide plus a GIPR×GCG conjugate.

FIG. 9 depicts the structure of a molecule provided herein. The molecule comprises a polypeptide agonist of GCGR (SEQ ID NO: 1871, which shares a linear peptide sequence with SEQ ID NO: 1587) covalently linked via an amide bond formed between an ε-amino group of a lysine residue at position 28 and the C-terminus of a polypeptide linker (SEQ ID NO: 1872, which shares a linear peptide sequence with SEQ ID NO: 1628), wherein the N-terminus of the polypeptide linker has been bromoacetylated to permit further conjugation to an anti-GIPR antibody via an alkylation reaction with a thiol group of a cysteine residue. For example, in GIPR×GCG conjugate 51671, two molecules with the structure depicted in FIG. 9 are covalently linked to an anti-GIPR antibody comprising two heavy chains, each comprising the amino acid sequence of SEQ ID NO: 1571, and two light chains, each comprising the amino acid sequence of SEQ ID NO: 388, wherein each molecule of FIG. 9 is covalently linked to the anti-GIPR antibody via a thiol-bromoacetyl reaction between the bromoacetylated N-terminus and a thiol group of a cysteine residue at position 275 of each heavy chain, which results in the formation of a thioether linkage that comprises a sulfur atom of the cysteine residue (one molecule per heavy chain).

FIG. 10 depicts the partial structure of a molecule provided herein comprising SEQ ID NOs: 1863 and 1864. The squiggly line represents a connection point between the partial structure and a sulfur atom of a cysteine residue of an anti-GIPR antibody described herein.

FIG. 11 depicts the structure of a molecule provided herein. The molecule comprises a polypeptide agonist of GCGR (SEQ ID NO: 1873, which shares a linear peptide sequence with SEQ ID NO: 1626) covalently linked via an amide bond formed between an ε-amino group of a lysine residue at position 28 and the C-terminus of a polypeptide linker (SEQ ID NO: 1874, which shares a linear peptide sequence with SEQ ID NO: 1629), wherein the N-terminus of the polypeptide linker has been bromoacetylated to permit further conjugation to an anti-GIPR antibody via a bromoacetyl-thiol reaction with a thiol group of a cysteine residue.

FIG. 12 depicts the partial structure of a molecule provided herein comprising SEQ ID NOs: 1865 and 1866. The squiggly line represents a connection point between the partial structure and a sulfur atom of a cysteine residue of an anti-GIPR antibody described herein.

FIG. 13 depicts the structure of a molecule provided herein. The molecule comprises a polypeptide agonist of GCGR (SEQ ID NO: 1875, which shares a linear peptide sequence with SEQ ID NO: 1626) covalently linked via an amide bond formed between an ε-amino group of a lysine residue at position 28 and the C-terminus of a polypeptide linker (SEQ ID NO: 1876, which shares a linear peptide sequence with SEQ ID NO: 1628), wherein the N-terminus of the polypeptide linker has been bromoacetylated to permit further conjugation to an anti-GIPR antibody via a bromoacetyl-thiol reaction with a thiol group of a cysteine residue.

FIG. 14 depicts the partial structure of a molecule provided herein comprising SEQ ID NOs: 1867 and 1868. The squiggly line represents a connection point between the partial structure and a sulfur atom of a cysteine residue of an anti-GIPR antibody described herein.

FIG. 15 depicts the structure of a molecule provided herein. The molecule comprises a polypeptide agonist of GCGR (SEQ ID NO: 1877, which shares a linear peptide sequence with SEQ ID NO: 1825) covalently linked via an amide bond formed between an ε-amino group of a lysine residue at position 28 and the C-terminus of a polypeptide linker (SEQ ID NO: 1878, which shares a linear peptide sequence with SEQ ID NO: 1628), wherein the N-terminus of the polypeptide linker has been bromoacetylated to permit further conjugation to an anti-GIPR antibody via a bromoacetyl-thiol reaction with a thiol group of a cysteine residue.

FIG. 16 depicts the partial structure of a molecule provided herein comprising SEQ ID NOs: 1869 and 1870. The squiggly line represents a connection point between the partial structure and a sulfur atom of a cysteine residue of an anti-GIPR antibody described herein.

### DETAILED DESCRIPTION

Disclosed herein are polypeptides having activity as agonists of a glucagon receptor, molecules comprising such polypeptides, pharmaceutical compositions comprising the polypeptides and molecules, and uses and methods in weight management and treating disorders, including obesity, with the polypeptides, molecules, and pharmaceutical compositions described herein.

### Definitions

The following definitions are provided to assist in understanding the scope of this disclosure. Unless defined otherwise, all technical and scientific terms used herein have the same meaning as is commonly understood by one of ordinary skill in the art to which this disclosure belongs.

As used herein, the terms “a” and “an” mean “one or more” unless specifically indicated otherwise. Additionally, “one or more” and “at least one” are used interchangeably herein. Furthermore, unless otherwise required by context, singular terms shall include pluralities, and plural terms shall include the singular.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges also encompassed within the disclosure, subject to any specifically excluded limit in the stated range. Unless otherwise required by context, numeric ranges are inclusive of the numbers defining the range (i.e., the endpoints). Where the stated range includes one or both of the limits, ranges excluding either or both of those included limits are also included in the disclosure.

Other than in the Examples, or where otherwise indicated, all numbers expressing quantities (e.g., of ingredients or reaction conditions) used herein should be understood as modified in all instances by the term “about.” As used herein, “about,” when used in connection with a measurable numerical variable, refers to the indicated value of the variable and to all values of the variable that are within the experimental error of the indicated value (e.g., within the 95% confidence interval for the mean) or +10% of the indicated value, whichever is greater.

Generally, nomenclatures used in connection with, and techniques of, cell and tissue culture, molecular biology, immunology, microbiology, genetics, and protein and nucleic acid chemistry and hybridization described herein are those well-known and commonly used in the art. For example, the methods and techniques of the present application (e.g., recombinant polypeptide and nucleic acid methods) are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001), Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Illustratively, protein purification methods that can be employed to isolate a polypeptide, as well as associated materials and reagents, are known in the art, and additional purification methods that may be useful for isolating a polypeptide can be found in references such as Bootcov MR, 1997, Proc. Natl. Acad. Sci. USA 94:11514-9, Fairlie W D, 2000, Gene 254: 67-76. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Illustratively, standard techniques can be used for chemical syntheses, chemical analyses, pharmaceutical preparation, formulation, and delivery, and treatment of patients. It should be understood that the subject matter of this disclosure is not limited to the particular methodology, protocols, and reagents, etc., described herein and as such may vary.

The term “naturally occurring,” as used herein in connection with biological materials such as polypeptides, nucleic acids, host cells, and the like, refers to materials which are found in nature.

As used herein, a “recombinant protein” is a protein made using recombinant techniques, i.e., through the expression of a recombinant nucleic acid, as described herein. Methods and techniques for the production of recombinant proteins are well-known in the art.

As used herein, the terms “amino acid” and “residue” are used interchangeably and, when used in the context of a polypeptide, refer to both naturally occurring and synthetic amino acids, as well as amino acid analogs, amino acid mimetics, and non-naturally occurring amino acids that are chemically similar to the naturally occurring amino acids.

As used herein, a “naturally occurring amino acid” is an amino acid that is encoded by the genetic code, as well as those amino acids that are encoded by the genetic code that are modified after synthesis, such as, e.g., hydroxyproline, γ-carboxyglutamate, and O-phosphoserine. An amino acid analog is a compound that has the same basic chemical structure as a naturally occurring amino acid, i.e., an a carbon that is bound to a hydrogen, a carboxyl group, an amino group, and an R group, e.g., homoserine, norleucine, methionine sulfoxide, or methionine methyl sulfonium. Such analogs can have modified R groups (e.g., norleucine) or modified peptide backbones, but will retain the same basic chemical structure as a naturally occurring amino acid.

Naturally occurring residues can be divided into classes based on common side chain properties:
1) hydrophobic: norleucine, Met, Ala, Val, Leu, Ile;
2) neutral hydrophilic: Cys, Ser, Thr;
3) acidic: Asp, Glu;
4) basic: Asn, Gln, His, Lys, Arg;
5) residues that influence chain orientation: Gly, Pro; and
6) aromatic: Trp, Tyr, Phe.

Additional groups of amino acids can also be formulated using the principles described in, e.g., Creighton (1984) PROTEINS: STRUCTURE AND MOLECULAR PROPERTIES (2d Ed. 1993), W.H. Freeman and Company. In some instances, it can be useful to further characterize substitutions based on two or more of such features (e.g., substitution with a “small polar” residue, such as a Thr residue, can represent a highly conservative substitution in an appropriate context).

As used herein, a “conservative amino acid substitution” can involve a substitution of a native amino acid residue (i.e., a residue found in a given position of a reference polypeptide sequence) with a non-native residue (i.e., a residue that is not found in a given position of the reference sequence) such that there is little or no effect on the polarity or charge of the amino acid residue at that position. Conservative amino acid substitutions also encompass non-naturally occurring amino acid residues that are typically incorporated by chemical peptide synthesis rather than by synthesis in biological systems. These include peptidomimetics, and other reversed or inverted forms of amino acid moieties.

Conservative substitutions can involve the exchange of a member of one of these classes for another member of the same class. Non-conservative substitutions can involve the exchange of a member of one of these classes for a member from another class.

Synthetic, rare, or modified amino acid residues having known similar physiochemical properties to those of an above-described grouping can be used as a “conservative” substitute for a particular amino acid residue in a sequence. For example, a D-Arg residue may serve as a substitute for a typical L-Arg residue. It also can be the case that a particular substitution can be described in terms of two or more of the above described classes (e.g., a substitution with a small and hydrophobic residue means substituting one amino acid with a residue(s) that is found in both of the above-described classes or other synthetic, rare, or modified residues that are known in the art to have similar physiochemical properties to such residues meeting both definitions).

Conservative substitutions can be determined by considering the hydropathic index of amino acids. The hydropathic profile of a protein is calculated by assigning each amino acid a numerical value (“hydropathy index”) and then repetitively averaging these values along the peptide chain. Each amino acid has been assigned a hydropathic index on the basis of its hydrophobicity and charge characteristics, e.g.: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic profile in conferring interactive biological function on a protein is understood in the art (see, e.g., Kyte et al., 1982, J. Mol. Biol. 157:105-131). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In some embodiments, those which are within ±1 are included, and in some embodiments, those within ±0.5 are included.

It is also understood in the art that the substitution of like amino acids can be made effectively on the basis of hydrophilicity, particularly where the biologically functional protein or peptide thereby created is intended for use in immunological embodiments. In some embodiments, the greatest local average hydrophilicity of a protein, as governed by the hydrophilicity of its adjacent amino acids, correlates with its immunogenicity and antigen-binding or immunogenicity, that is, with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within 2 is included, in other embodiments, those which are within 1 are included, and in still other embodiments, those within 0.5 are included. In some instances, one may also identify epitopes from primary amino acid sequences on the basis of hydrophilicity. These regions are also referred to as “epitopic core regions.”

Non-limiting examples of conservative amino acid substitutions are set forth in Table 1.

TABLE 1
Non-Limiting Example Conservative Amino Acid Substitutions

| | | Specific Example |
|---|---|---|
| Original | Example Substitutions | Substitutions |
| Ala (A) | Val, Leu, Ile | Val |
| Arg (R) | Lys, Gln, Asn | Lys |
| Asn (N) | Gln, His, Asp, Lys, Arg | Gln |
| Asp (D) | Glu, Asn | Glu |
| Cys (C) | Ser, Ala | Ser |
| Gln (Q) | Asn, Glu | Asn |
| Glu (E) | Asp, Gln | Asp |
| Gly (G) | Ala | Ala |
| His (H) | Asn, Gln, Lys, Arg | Arg |
| Ile (I) | Leu, Val, Met, Ala, Phe | Leu |
| Leu (L) | Norleucine, Ile, Val, Met, Ala | Ile |
| Lys (K) | Arg, Gln, Asn | Arg |
| Met (M) | Leu, Phe, Ile | Leu |
| Phe (F) | Leu, Val, Ile, Ala, Tyr | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala | Leu |

As used herein, an “amino acid mimetic” is a chemical compound that has a structure that is different from the general chemical structure of an amino acid but that functions in a manner similar to a naturally occurring amino acid. Examples include, but are not limited to, a methacryloyl or acryloyl derivative of an amide, β-, γ-, δ-imino acids (such as, e.g., piperidine-4-carboxylic acid), and the like.

As used herein, a “non-naturally occurring amino acid” is a compound that has the same basic chemical structure as a naturally occurring amino acid but is not incorporated into a growing polypeptide chain by the translation complex. “Non-naturally occurring amino acid” also refers to, but is not limited to, amino acids that occur by modification (e.g., post-translational modification(s)) of a naturally encoded amino acid (including but not limited to, the 20 common amino acids) but are not themselves naturally incorporated into a growing polypeptide chain by the translation complex. A non-limiting list of examples of non-naturally occurring amino acids that can be inserted into a polypeptide sequence or substituted for a wild-type residue in a polypeptide sequence include β-amino acids, homoamino acids, cyclic amino acids, and amino acids with derivatized side chains. Examples include (in the L-form or D-form; abbreviated as in parentheses) but are not limited to: citrulline (Cit), homocitrulline (hCit), Nα-methylcitrulline (NMeCit), Nα-methylhomocitrulline (Nα-MeHoCit), ornithine (Orn), Nα-Methylornithine (Nα-MeOrn or NMeOrn), sarcosine (Sar), homolysine (hLys or hK), homoarginine (hArg or hR), homoglutamine (hQ), Nα-methylarginine (NMeR), Nα-methylleucine (Nα-MeL or NMeL), N-methylhomolysine (NMeHoK), Nα-methylglutamine (NMeQ), norleucine (Nle), norvaline (Nva), 1,2,3,4-tetrahydroisoquinoline (Tic), Octahydroindole-2-carboxylic acid (Oic), 3-(1-naphthyl)alanine (1-Nal), 3-(2-naphthyl)alanine (2-Nal), 1,2,3,4-tetrahydroisoquinoline (Tic), 2-indanylglycine (IgI), para-iodophenylalanine (pI-Phe), para-aminophenylalanine (4AmP or 4-Amino-Phe), 4-guanidino phenylalanine (Guf), glycyllysine (abbreviated “K (NF-glycyl)” or “K(glycyl)” or “K(gly)”), nitrophenylalanine (nitrophe), aminophenylalanine (aminophe or Amino-Phe), benzylphenylalanine (benzylphe), γ-carboxyglutamic acid (γ-carboxyglu), hydroxyproline (hydroxypro), p-carboxyl-phenylalanine (Cpa), α-aminoadipic acid (Aad), Nα-methyl valine (NMeVal), N-α-methyl leucine (NMeLeu), Nα-methylnorleucine (NMeNle), cyclopentylglycine (Cpg), cyclohexylglycine (Chg), acetylarginine (acetylarg), a, β-diaminopropionoic acid (Dpr), a, γ-diaminobutyric acid (Dab), diaminopropionic acid (Dap), cyclohexylalanine (Cha), 4-methyl-phenylalanine (MePhe), β, β-diphenyl-alanine (BiPhA), aminobutyric acid (Abu), 4-phenyl-phenylalanine (or biphenylalanine; 4Bip), α-amino-isobutyric acid (Aib), beta-alanine, beta-aminopropionic acid, piperidinic acid, aminocaprioic acid, aminoheptanoic acid, aminopimelic acid, desmosine, diaminopimelic acid, N-ethylglycine, N-ethylaspargine, hydroxylysine, allo-hydroxylysine, isodesmosine, allo-isoleucine, N-methylglycine, N-methylisoleucine, N-methylvaline, 4-hydroxyproline (Hyp), γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-methylarginine, 4-amino-O-phthalic acid (4APA), and other similar amino acids, and derivatized forms of any of those specifically listed.

As used herein, the term “antigen” refers to a molecule or a portion of a molecule capable of being bound by a selective binding agent, such as an antigen binding protein (including, e.g., an antibody), and additionally capable of being used in an animal to produce antibodies capable of binding to that antigen. An antigen may possess one or more epitopes that are capable of interacting with different antigen binding proteins, e.g., antibodies.

As used herein, an “antigen-binding region” refers to a protein, or a portion of a protein, that specifically binds a specified antigen. For example, that portion of an antigen-binding protein that contains the amino acid residues that interact with an antigen and confer on the antigen-binding protein its specificity and affinity for the antigen is referred to as “antigen binding region.” An antigen-binding region typically includes one or more “complementary binding regions” (“CDRs”) of an immunoglobulin, single-chain immunoglobulin, or camelid antibody. Certain antigen binding regions also include one or more “framework” regions. A “CDR” is an amino acid sequence that contributes to antigen binding specificity and affinity. “Framework” regions can aid in maintaining the proper conformation of the CDRs to promote binding between the antigen-binding region and an antigen.

As used herein, the term “polypeptide” refers to a polymer of amino acid residues. Polypeptides comprising between two and fifty amino acids may also be referred to as “peptides” herein. “Polypeptide” further encompasses an amino acid polymer in which one or more amino acid residues is an analog or mimetic of a corresponding naturally occurring amino acid, as well as to naturally occurring amino acid polymers. The term can also encompass an amino acid polymer that have been modified, e.g., by the addition of carbohydrate residues to form glycoproteins, or phosphorylated. Polypeptides can be produced by a naturally-occurring and non-recombinant cell, or polypeptides can be produced by a genetically-engineered or recombinant cell, and comprise molecules having the amino acid sequence of the native protein, or molecules having deletions from, additions to, and/or substitutions of one or more amino acids of the native sequence. The terms “polypeptide” and “protein” are used interchangeably herein.

As used herein, the term “polypeptide fragment” refers to a polypeptide that has an amino-terminal deletion, a carboxyl-terminal deletion, and/or an internal deletion as compared with a reference polypeptide. Such fragments may also contain modified amino acids as compared with the reference polypeptide. In certain embodiments, fragments are five to 500 amino acids long. For example, fragments may be at least 5, 6, 8, 10, 14, 20, 50, 70, 100, 110, 150, 200, 250, 300, 350, 400, or 450 amino acids long.

As used herein, a “variant” of a polypeptide (e.g., an antigen-binding protein such as an antibody) comprises an amino acid sequence wherein one or more amino acid residues are inserted into, deleted from and/or substituted into the amino acid sequence relative to a reference polypeptide sequence. Variants include fusion proteins.

As used herein, a “derivative” of a polypeptide is a polypeptide (e.g., an antigen binding protein such as an antibody) that has been chemically modified in some manner distinct from insertion, deletion, or substitution variants, such as, e.g., via conjugation to another chemical moiety.

As used herein, the terms “chemical derivative” or “chemically derivatized,” with respect to a polypeptide, refer to a polypeptide that comprises one or more residues that have been chemically derivatized by reaction of a functional side group. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups, or formyl groups. For example, free carboxyl groups can be derivatized to form salts, methyl and ethyl esters, or other types of esters or hydrazides. Additionally, free hydroxyl groups can be derivatized to form O-acyl or O-alkyl derivatives, and the imidazole nitrogen of histidine can be derivatized to form Nim-benzylhistidine.

Non-limiting examples of derivatizations also include the following:

Chemical modification of the amino terminal of the polypeptide: In some embodiments, the N-terminus can be acylated or modified to a substituted amine, or derivatized with another functional group, such as an aromatic moiety (e.g., an indole acid, benzyl (Bzl or Bn), dibenzyl (DiBzl or Bn₂), or benzyloxycarbonyl (Cbz or Z)), N,N-dimethylglycine, or creatine). For example, in some embodiments, an acyl moiety, such as, but not limited to, a formyl, acetyl (Ac), propanoyl, butanyl, heptanyl, hexanoyl, octanoyl, or nonanoyl, can be covalently linked to the N-terminal end of the polypeptide. Other example N-terminal derivative groups include, but are not limited to, —NRR¹ (other than —NH₂), —NRC(O)R¹, —NRC(O)OR¹, —NRS(O)₂R¹, —NHC(O)NHR¹, succinimide, or benzyloxycarbonyl-NH— (Cbz-NH—), wherein R and R¹ are each independently hydrogen or C₁₋₄ alkyl and wherein the phenyl ring may be substituted with 1 to 3 substituents independently selected from C₁₋₄ alkyl, C₁₋₄ alkoxy, chloro, and bromo.

Bond substitutions: In some embodiments, one or more peptidyl [—C(O)NR—] linkages (bonds) between amino acid residues can be replaced by a non-peptidyl linkage. Example non-peptidyl linkages include, but are not limited to, —CH₂-carbamate [—CH₂—OC(O)NR—], phosphonate, —CH₂-sulfonamide [—CH₂—S(O)₂NR—], urea [—NHC(O)NH—], —CH₂-secondary amine, and alkylated peptide [—C(O)NR⁶—, wherein R⁶ is C₁₋₄ alkyl].

Derivatization of one or more individual amino acid residues: In some embodiments, lysinyl residues and amino terminal residues can be reacted with succinic or other carboxylic acid anhydrides, which reverse the charge of the lysinyl residues. Other suitable reagents for derivatizing alpha-amino-containing residues include, but are not limited to, imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

In some embodiments, arginyl residues can be modified by reaction with any one or more of several conventional reagents, including phenylglyoxal, 2,3-butanedione, 1,2-cyclohexanedione, and ninhydrin. Derivatization of arginyl residues requires that the reaction be performed in alkaline conditions because of the high pKa of the guanidine functional group. Furthermore, these reagents can react with the groups of lysine as well as the arginine epsilon-amino group.

Specific modification of tyrosyl residues has been studied extensively, with particular interest in introducing spectral labels into tyrosyl residues by reaction with aromatic diazonium compounds or tetranitromethane. Most commonly, N-acetylimidizole and tetranitromethane are used to form 0-acetyl tyrosyl species and 3-nitro derivatives, respectively.

In some embodiments, carboxyl sidechain groups (aspartyl or glutamyl) can be selectively modified by reaction with carbodiimides (R′—N═C═N—R′) such as 1-cyclohexyl-3-(2-morpholinyl-(4-ethyl) carbodiimide or 1-ethyl-3-(4-azonia-4,4-dimethylpentyl) carbodiimide. Furthermore, in some embodiment, aspartyl and glutamyl residues can be converted to asparaginyl and glutaminyl residues by reaction with ammonium ions.

In some embodiments, glutaminyl and asparaginyl residues can be deamidated to the corresponding glutamyl and aspartyl residues. In alternative embodiments, these residues are deamidated under mildly acidic conditions.

In some embodiments, cysteinyl residues can be replaced by amino acid residues or other moieties either to eliminate disulfide bonding or, conversely, to stabilize cross-linking. (See, e.g., Bhatnagar et al., J. Med. Chem., 39:3814-3819 (1996)).

Other possible modifications include, but are not limited to, hydroxylation of proline and lysine, phosphorylation of hydroxyl groups of seryl or threonyl residues, oxidation of the sulfur atom in Cys, methylation of the alpha-amino groups of lysine, arginine, and histidine side chains. Creighton, Proteins: Structure and Molecule Properties (W. H. Freeman & Co., San Francisco), 79-86 (1983).

As used herein, the term “alkyl” refers to a saturated straight chain hydrocarbon or saturated branched chain hydrocarbon containing the indicated number of carbon atoms. For example, C₃ alkyl means an alkyl group that has 3 carbon atoms (e.g., n-propyl or isopropyl). For example, a C₁₋₆ alkyl refers to an alkyl group having 1 to 6 carbon atoms. Where a range is indicated, all members of that range and all subgroups within that range are envisioned. For example, a C₁₋₆ alkyl includes alkyl groups having 1, 2, 3, 4, 5, or 6 carbon atoms (or any combination of the foregoing), as well as all subgroups in the indicated range (e.g., 1-2, 1-3, 1-4, 1-5, 1-6, 2-3, 2-4, 2-5, 2-6, 3-4, 3-5, 3-6, 4-5, 4-6, or 5-6 carbon atoms, or any combination of the foregoing ranges)). A “C₁₋₄ alkyl” includes, for example, methyl, ethyl, n-propyl, isopropyl, n-butyl, isobutyl, sec-butyl, or t-butyl. Nonlimiting examples of alkyl groups include methyl, ethyl, n-propyl, isopropyl, n-butyl, sec-butyl, isobutyl, tert-butyl, n-pentyl, and n-hexyl.

A molecule of the present disclosure that includes a polypeptide which is covalently linked, attached, or bound, either directly or indirectly through a linker moiety (e.g., a linker polypeptide), to another polypeptide, such as, e.g., an anti-GIPR antibody of the present disclosure, may be described herein as a “conjugate.”

As used herein, the term “thiol” refers to a —SH group.

As used herein, the terms “alkoxy” and “alkoxyl” are interchangeable and refer to an —O-alkyl group, where the alkyl group is as defined elsewhere herein. For example, a C₃alkoxy group means the alkoxy group has 3 carbon atoms (e.g., OCH₂CH₂CH₃). Where a range is indicated, all members of that range and all subgroups within that range are envisioned. For example, a C₁₋₆alkoxy includes alkoxy groups having 2, 3, 4, 5, or 6 carbon atoms, or any combination of the foregoing, as well as all subgroups in the indicated range (e.g., 2-3, 2-4, 2-5, 2-6, 3-4, 3-5, 3-6, 4-5, 4-6, and 5-6 carbon atoms, or any combination of the foregoing). Nonlimiting examples of alkoxy groups include methoxy, ethoxy, n-propoxy, 1-methylethyloxy (iso-propoxy), n-butoxy, isobutoxy, sec-butoxy, and tert-butoxy.

As used herein, the term “linker moiety” refers to a biologically acceptable peptidyl or non-peptidyl organic group that is covalently bound to a first molecule (e.g., a first polypeptide) and covalently joins or conjugates the molecule to a second molecule (e.g., a second polypeptide). Where the linker moiety consists of a polypeptide or a polypeptide derivative (e.g., a polypeptide that has been chemically modified at one or both of the N-terminus and C-terminus to incorporate a functional group that permits conjugation to the first or second molecule), it may be referred to as a “linker polypeptide” herein. For example, in some embodiments, a linker polypeptide may comprise an acetylated N-terminus (e.g., when a thiol-bromoacetyl reaction was used to conjugate the derivatized N-terminus of the linker polypeptide to a cysteine residue of a polypeptide by forming a thioether linkage that comprises a sulfur atom of the cysteine residue).

As used herein, the term “isolated polypeptide” refers to a polypeptide that has been separated from at least about 50 percent of polypeptides, lipids, carbohydrates, polynucleotides, or other materials with which the polypeptide is naturally found when isolated from a source cell. In some embodiments, the isolated polypeptide is substantially free from any other contaminating polypeptides or other contaminants that are found in its natural environment that would interfere with its therapeutic, diagnostic, prophylactic, or research use.

As used herein, the term “antigen-binding protein” refers to any protein that specifically binds a specified target antigen, such as a GIPR polypeptide (e.g., a human GIPR polypeptide such as those provided in SEQ ID NOs: 1577, 1578, or 1579). The term encompasses intact antibodies that comprise at least two full-length heavy chains and two full-length light chains, as well as derivatives, variants, fragments, and mutations thereof. An antigen-binding protein also includes domain antibodies such as nanobodies and scFvs as described further below.

An antigen-binding protein, such as, e.g., an anti-GIPR polypeptide, is said to “specifically bind” its target antigen when the antigen binding protein exhibits essentially background binding to non-target antigen molecules. An antigen binding protein that specifically binds a target antigen may, however, cross-react with target antigens from different species. Typically, an antigen binding protein specifically binds its target antigen when the dissociation constant (K_{D}) is ≤10⁻⁷ M as measured via a surface plasma resonance technique (e.g., BIACore, GE-Healthcare Uppsala, Sweden) or Kinetic Exclusion Assay (KinExA, Sapidyne, Boise, Idaho). An antigen-binding protein specifically binds its target antigen with “high affinity” when the K_{D} is ≤5×10⁻⁹ M, and with “very high affinity” when the K_{D} is ≤5×10⁻¹⁰ M, as measured using methods described.

As used herein, the term “epitope” is the portion of a molecule that is bound by an antigen-binding protein (e.g., an antibody). The term includes any determinant capable of specifically binding to an antigen-binding protein, such as an antibody. An epitope can be contiguous or non-contiguous (discontinuous) (e.g., amino acid residues that are not contiguous to one another in an amino acid sequence but that within in context of the molecule are bound by the antigen-binding protein). A conformational epitope is an epitope that exists within the conformation of an active protein but is not present in a denatured protein. In some embodiments, epitopes may be mimetic in that they comprise a three dimensional structure that is similar to an epitope used to generate the antigen-binding protein, yet comprise none or only some of the amino acid residues found in that epitope used to generate the antigen binding protein. More often, epitopes reside on proteins, but in some instances may reside on other kinds of molecules, such as nucleic acids. Epitope determinants may include chemically active surface groupings of molecules such as amino acids, sugar side chains, phosphoryl or sulfonyl groups, and may have specific three dimensional structural characteristics, and/or specific charge characteristics. Generally, antigen-binding proteins specific for a particular target antigen will preferentially recognize an epitope on the target antigen in a complex mixture of proteins and/or macromolecules.

As used herein, a “bivalent antigen-binding protein” (e.g., a bivalent antibody) comprises two antigen binding regions. In some instances, the two binding regions have the same antigen specificities. Bivalent antigen binding proteins and bivalent antibodies may be bispecific.

As used herein, a “multispecific antigen-binding protein” (e.g., a multispecific antibody) is an antigen-binding protein that targets more than one antigen or epitope.

As used herein, a “bispecific,” “dual-specific” or “bifunctional” antigen-binding protein (e.g., antibody) is a hybrid antigen-binding protein having two different antigen binding sites. Bispecific antigen-binding proteins are a subclass of multispecific antigen-binding proteins and may be produced by a variety of methods including, but not limited to, fusion of hybridomas or linking of Fab′ fragments. See, e.g., Songsivilai and Lachmann, 1990, Clin. Exp. Immunol. 79:315-321; Kostelny et al., 1992, J. Immunol. 148:1547-1553. The two binding sites of a bispecific antigen binding protein will bind to two different epitopes, which may reside on the same or different protein targets.

As used herein, the term “antibody” refers to an intact immunoglobulin of any isotype, and includes, for instance, chimeric, humanized, fully human, and bispecific antibodies. An “antibody” as such is a species of an antigen-binding protein. An antibody generally comprises two full-length heavy chains and two full-length light chains. Antibodies may be derived solely from a single source, or may be “chimeric,” that is, different portions of the antibody may be derived from two different antibodies as described further below.

As used herein, the term “light chain” or “immunoglobulin light chain” refers to a polypeptide comprising, from amino terminus (N-terminus) to carboxyl terminus (C-terminus), a single immunoglobulin light chain variable region (VL) and a single immunoglobulin light chain constant domain (CL). The immunoglobulin light chain constant domain (CL) can be a human kappa (κ) or human lambda (λ) constant domain.

As used herein, the term “heavy chain” or “immunoglobulin heavy chain” refers to a polypeptide comprising, from amino terminus (N-terminus) to carboxyl terminus (C-terminus), a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the two antibody heavy chains.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein. From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309(3):657-670; 2001).

As used herein, the term “immunologically functional fragment” (or simply “fragment” or “functional fragment”) of an antibody is an antigen-binding protein comprising a portion (regardless of how that portion is obtained or synthesized) of an antibody that lacks at least some of the amino acids present in a full-length chain but which is capable of specifically binding to the same antigen at the same epitope as the antibody. Such fragments are biologically active in that they bind specifically to the target antigen and can compete with other antigen binding proteins, including intact antibodies, for specific binding to a given epitope. These biologically active fragments may be produced by recombinant DNA techniques, or may be produced by enzymatic or chemical cleavage of antigen binding proteins, including intact antibodies. Immunologically functional immunoglobulin fragments include, but are not limited to, Fab, Fab′, and F(ab′)₂ fragments.

Papain digestion of antibodies produces two identical antigen-binding proteins, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CH1 domain and variable region (VH) of one immunoglobulin heavy chain. The heavy chain of a Fab molecule cannot form a disulfide bond with another heavy chain molecule. The “Fd fragment” comprises the VH and CH1 domains from an immunoglobulin heavy chain. The Fd fragment represents the heavy chain component of the Fab fragment.

As used herein, a “Fc fragment” or “Fc region” of an immunoglobulin generally comprises two constant domains, a CH2 domain and a CH3 domain, and optionally comprises a CH4 domain. The Fc region may be an Fc region from an IgG1, IgG2, IgG3, or IgG4 immunoglobulin. In some embodiments, the Fc region comprises CH2 and CH3 domains from a human IgG1 or human IgG2 immunoglobulin. The Fc region may retain effector function, such as C1q binding, complement dependent cytotoxicity (CDC), Fc receptor binding, antibody-dependent cell-mediated cytotoxicity (ADCC), and phagocytosis. In other embodiments, the Fc region may be modified to reduce or eliminate effector function.

As used herein, a “Fab′ fragment” contains one light chain and a portion of one heavy chain that contains the VH domain and the CH1 domain and also the region between the CH1 and CH2 domains, such that an interchain disulfide bond can be formed between the two heavy chains of two Fab′ fragments to form an F(ab′)₂ molecule.

As used herein, a “F(ab′)₂ fragment” contains two light chains and two heavy chains containing a portion of the constant region between the CH1 and CH2 domains, such that an interchain disulfide bond is formed between the two heavy chains. A F(ab′)₂ fragment thus is composed of two Fab′ fragments that are held together by a disulfide bond between the two heavy chains.

As used herein, a “Fv region” comprises the variable regions from both the heavy and light chains, but lacks the constant regions. The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

As used herein, a “single-chain variable fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

As used herein, a “nanobody” is the heavy chain variable region of a heavy-chain antibody. Such variable domains are the smallest fully functional antigen-binding fragment of such heavy-chain antibodies with a molecular mass of only 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks, and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only having the structure H2L2 (referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276:26285-90, 2001) and possess high stability in solution (Ewert et al., Biochemistry, Vol. 41:3628-36, 2002). Methods for generating antibodies having camelized heavy chains are described in, for example, U.S. Patent Publication Nos. 2005/0136049 and 2005/0037421. Alternative scaffolds can be made from human variable-like domains that more closely match the shark V-NAR scaffold and may provide a framework for a long penetrating loop structure.

As used herein, the term “heavy chain-only antibody” refers to an immunoglobulin protein consisting of two heavy chain polypeptides (such as, e.g., heavy chain polypeptides that are about 50-70 kDa each). A “heavy chain-only antibody” lacks the two light chain polypeptides found in a conventional antibody. Heavy-chain antibodies constitute about one-fourth of the IgG antibodies produced by the camelids, e.g., camels and llamas (Hamers-Casterman C., et al. Nature. 363, 446-448 (1993)). These molecules are formed by two heavy chains but are devoid of light chains. As a consequence, the variable antigen binding part is referred to as the VHH domain, and it represents the smallest naturally occurring, intact, antigen-binding site, being only around 120 amino acids in length (Desmyter, A., et al. J. Biol. Chem. 276, 26285-26290 (2001)). Heavy chain antibodies with a high specificity and affinity can be generated against a variety of antigens through immunization (van der Linden, R. H., et al. Biochim. Biophys. Acta. 1431, 37-46 (1999)), and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)). Sharks have also been shown to have a single VH-like domain in their antibodies, termed VNAR. (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003).)

In some embodiments, a “heavy chain-only antibody” is a dimeric antibody comprising a VH antigen-binding domain and the CH2 and CH3 constant domains, in the absence of the CH1 domain. In some embodiments, a heavy chain-only antibody is composed of a variable region antigen-binding domain composed of framework 1, CDR1, framework 2, CDR2, framework 3, CDR3, and framework 4. In some embodiments, a heavy chain-only antibody is composed of an antigen-binding domain, at least part of a hinge region, and CH2 and CH3 domains. In some embodiments, a heavy chain-only antibody is composed of an antigen-binding domain, at least part of a hinge region, and a CH2 domain. In some embodiments, a heavy chain-only antibody is composed of an antigen-binding domain, at least part of a hinge region, and a CH3 domain.

Heavy chain-only antibodies in which the CH2 and/or CH3 domain is truncated are also included herein. The heavy chain-only antibodies described herein may belong to the IgG subclass, but heavy chain-only antibodies belonging to other subclasses, such as IgM, IgA, IgD and IgE subclass, are also included herein. In some embodiments, a heavy chain-only antibody may belong to the IgG1, IgG2, IgG3, or IgG4 subtype, e.g., the IgG1 or IgG4 subtype. In some embodiments, a heavy chain antibody-only is of the IgG1 or IgG4 subtype, wherein one or more of the CH domains is modified to alter an effector function of the antibody. In some embodiments, a heavy chain-only antibody is of the IgG4 subtype, wherein one or more of the CH domains is modified to alter an effector function of the antibody. In some embodiments, a heavy chain-only antibody is of the IgG1 subtype, wherein one or more of the CH domains is modified to alter an effector function of the antibody. Modifications of CH domains that alter effector function are further described herein. Non-limiting examples of heavy-chain-only antibodies are described, for example, in WO2018/039180, the disclosure of which is incorporated herein by reference herein in its entirety.

As used herein, the term “three-chain antibody like molecule” or “TCA” refers to an antibody-like molecule comprising, consisting essentially of, or consisting of three polypeptide subunits, two of which comprise, consist essentially of, or consist of one heavy and one light chain of an antibody, or antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy-chain only antibody comprising an Fc portion comprising CH2 and/or CH3 and/or CH4 domains, in the absence of a CH1 domain, and one or more antigen binding domains (such as, e.g., two antigen binding domains) that binds an epitope of a second antigen or a different epitope of the first antigen, where such binding domain is derived from or has sequence identity with the variable region of an antibody heavy or light chain. Parts of such variable region may be encoded by VH and/or VL gene segments, D and JH gene segments, or JL gene segments. The variable region may be encoded by rearranged VHDJH, VLDJH, VHJL, or VLJL gene segments.

As used herein, the term “identical,” in the context of two or more nucleic acids or polypeptide sequences, refer to two or more sequences or subsequences that are the same.

As used herein, “percent identity” means the percent of identical residues between the amino acids or nucleotides in compared molecules and is calculated based on the size of the smallest of the molecules being compared. For these calculations, gaps in alignments (if any) can be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), (1988) New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., (1987) Sequence Analysis in Molecular Biology, New York: Academic Press; Sequence Analysis Primer, (Gribskov, M. and Devereux, J., eds.), 1991, New York: M. Stockton Press; and Carillo et al., (1988) SIAM J. Applied Math. 48:1073.

In calculating percent identity, the sequences being compared are aligned in a way that gives the largest match between the sequences. The computer program used to determine percent identity is the GCG program package, which includes GAP (Devereux et al., (1984) Nucl. Acid Res. 12:387; Genetics Computer Group, University of Wisconsin, Madison, WI). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In some embodiments, a standard comparison matrix (see, Dayhoff et al., (1978) Atlas of Protein Sequence and Structure 5:345-352 for the PAM 250 comparison matrix; Henikoff et al., (1992) Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program are the following:
Algorithm: Needleman et al., 1970, J. Mol. Biol. 48:443-453;
Comparison matrix: BLOSUM 62 from Henikoff et al., 1992, supra;
Gap Penalty: 12 (but with no penalty for end gaps)
Gap Length Penalty: 4
Threshold of Similarity: 0

Certain alignment schemes for aligning two amino acid sequences can result in matching of only a short region of the two sequences, and this small, aligned region can have very high sequence identity even though there is no significant relationship between the two full-length sequences. Accordingly, the selected alignment method (e.g., the GAP program) can be adjusted if so desired to result in an alignment that spans at least 50 contiguous amino acids of the target polypeptide.

As used herein, the terms “GIP,” “gastric inhibitory polypeptide,” “glucose-dependent insulinotropic polypeptide,” and “GIP ligand” are used interchangeably and refer to a naturally-occurring wild-type polypeptide expressed in a mammal, such as a human or a mouse, and include naturally occurring alleles (e.g., naturally occurring allelic forms of human GIP protein). For the purposes of this disclosure, the term “GIP” can be used interchangeably to refer to any mature GIP polypeptide.

The 42 amino acid sequence of mature human GIP is:

(SEQ ID NO: 1582)
YAEGTFISDY SIAMDKIHQQ DFVNWLLAQK GKKNDWKHINI TQ.

The 42 amino acid sequence of mature murine GIP is:

(SEQ ID NO: 1583)
YAEGTFISDY SIAMDKIRQQ DFVNWLLAQR GKKSDWKHNI TQ.

The 42 amino acid sequence of mature rat GIP is:

(SEQ ID NO: 1584)
YAEGTFISDY SIAMDKIRQQ DFVNWLLAQK GKKNDWKHINL TQ.

Additionally, as used herein, the terms “GIPR polypeptide” and “GIPR protein” are used interchangeably and refer to a naturally-occurring wild-type polypeptide expressed in a mammal, such as a human or a mouse, and include naturally occurring alleles (e.g., naturally occurring allelic forms of human GIPR protein). For the purposes of this disclosure, the term “GIPR polypeptide” can be used interchangeably to refer to any full-length GIPR polypeptide, e.g., SEQ ID NO: 1577, which consists of 466 amino acid residues, or SEQ ID NO: 1578, which consists of 430 amino acid residues, or SEQ ID NO: 1579, which consists of 493 amino acid resides, or SEQ ID NO: 1580, which consists of 460 amino acids residues, or SEQ ID NO: 1581, which consists of 230 amino acids residues.

The 466 amino acid sequence of human GIPR is (Volz et al., FEBS Lett. 373:23-29 (1995); NCBI Reference Sequence NP_0001555):

(SEQ ID NO: 1577)
MTTSPILQLL LRLSLCGLLL QRAETGSKGQ TAGELYQRWE
RYRRECQETL AAAEPPSGLA CNGSFDMYVC WDYAAPNATA
RASCPWYLPW HHHVAAGFVL RQCGSDGQWG LWRDHTQCEN
PEKNEAFLDQ RLILERLQVM YTVGYSLSLA TLLLALLILS
LFRRLHCTRN YIHINLFTSF MLRAAAILSR DRLLPRPGPY
LGDQALALWN QALAACRTAQ IVTQYCVGAN YTWLLVEGVY
LHSLLVLVGG SEEGHFRYYL LLGWGAPALF VIPWVIVRYL
YENTQCWERN EVKAIWWIIR TPILMTILIN FLIFIRILGI
LLSKLRTRQM RCRDYRLRLA RSTLTLVPLL GVHEVVFAPV
TEEQARGALR FAKLGFEIFL SSFQGFLVSV LYCFINKEVQ
SEIRRGWHHC RLRRSLGEEQ RQLPERAFRA LPSGSGPGEV
PTSRGLSSGT LPGPGNEASR ELESYC

A 430 amino acid isoform of human GIPR (isoform X1), predicted by automated computational analysis, has the sequence (NCBI Reference Sequence XP_005258790):

(SEQ ID NO: 1578)
MTTSPILQLL LRLSLCGLLL QRAETGSKGQ TAGELYQRWE
RYRRECQETL AAAEPPSVAA GFVLRQCGSD GQWGLWRDHT
QCENPEKNEA FLDQRLILER LQVMYTVGYS LSLATLLLAL
LILSLFRRLH CTRNYIHINL FTSFMLRAAA ILSRDRLLPR
PGPYLGDQAL ALWNQALAAC RTAQIVTQYC VGANYTWLLV
EGVYLHSLLV LVGGSEEGHF RYYLLLGWGA PALFVIPWVI
VRYLYENTQC WERNEVKAIW WIIRTPILMT ILINFLIFIR
ILGILLSKLR TRQMRCRDYR LRLARSTLTL VPLLGVHEVV
FAPVTEEQAR GALRFAKLGF EIFLSSFQGF LVSVLYCFIN
KEVQSEIRRG WHHCRLRRSL GEEQRQLPER AFRALPSGSG
PGEVPTSRGL SSGTLPGPGN EASRELESYC

493 amino acid isoform of human GIPR, produced by alternative splicing, as the sequence (Gremlich et al., Diabetes 44:1202-8 (1995); UniProtKB Sequence Identifier: P48546-2):

(SEQ ID NO: 1579)
MTTSPILQLL LRLSLCGLLL QRAETGSKGQ TAGELYQRWE
RYRRECQETL AAAEPPSGLA CNGSFDMYVC WDYAAPNATA
RASCPWYLPW HHHVAAGFVL RQCGSDGQWG LWRDHTQCEN
PEKNEAFLDQ RLILERLQVM YTVGYSLSLA TLLLALLILS
LFRRLHCTRN YIHINLFTSF MLRAAAILSR DRLLPRPGPY
LGDQALALWN QALAACRTAQ IVTQYCVGAN YTWLLVEGVY
LHSLLVLVGG SEEGHFRYYL LLGWGAPALF VIPWVIVRYL
YENTQCWERN EVKAIWWIIR TPILMTILIN FLIFIRILGI
LLSKLRTRQM RCRDYRLRLA RSTLTLVPLL GVHEVVFAPV
TEEQARGALR FAKLGFEIFL SSFQGFLVSV LYCFINKEVG
RDPAAAPALW RRRGTAPPLS AIVSQVQSEI RRGWHHCRLR
RSLGEEQRQL PERAFRALPS GSGPGEVPTS RGLSSGTLPG
PGNEASRELE SYC

The 460 amino acid sequence of murine GIPR is (NCBI Reference Sequence: NP_001074284; UniProtKB/Swiss-Prot Q0P543-1); see Vassilatis et al., PNAS USA 2003, 100:4903-4908.

(SEQ ID NO: 1580)
MPLRLLLLLL WLWGLQWAET DSEGQTTTGE LYQRWEHYGQ
ECQKMLETTE PPSGLACNGS FDMYACWNYT AANTTARVSC
PWYLPWFRQV SAGFVFRQCG SDGQWGSWRD HTQCENPEKN
GAFQDQTLIL ERLQIMYTVG YSLSLTTLLL ALLILSLFRR
LHCTRNYIHM NLFTSFMLRA AAILTRDQLL PPLGPYTGDQ
APTPWNQALA ACRTAQIMTQ YCVGANYTWL LVEGVYLHHL
LVIVGRSEKG HFRCYLLLGW GAPALFVIPW VIVRYLRENT
QCWERNEVKA IWWIIRTPIL ITILINFLIF IRILGILVSK
LRTRQMRCPD YRLRLARSTL TLVPLLGVHE VVFAPVTEEQ
VEGSLRFAKL AFEIFLSSFQ GFLVSVLYCFINKEVQSEIRQ
GWRHRRLRLS LQEQRPRPHQ ELAPRAVPLS SACREAAVGN
ALPSGMLHVP GDEVLESYC

A 230 amino acid isoform of murine GIPR, produced by alternative splicing, has the sequence (Gerhard et al., Genome Res, 14:2121-2127 (2004); NCBI Reference Sequence: AAI20674):

(SEQ ID NO: 1581)
MPLRLLLLLL WLWGLQWAET DSEGQTTTGE LYQRWEHYGQ
ECQKMLETTE PPSGLACNGS FDMYACWNYT AANTTARVSC
PWYLPWFRQV SAGFVFRQCG SDGQWGSWRD HTQCENPEKN
GAFQDQTLIL ERLQIMYTVG YSLSLTTLLL ALLILSLFRR
LHCTRNYIHM NLFTSFMLRA AAILTRDQLL PPLGPYTGDQ
APTPWNQVLH RLLPGGTKTF PIYFRTFPHH

As stated herein, the term “GIPR polypeptide” encompasses naturally occurring GIPR polypeptide sequences, e.g., human amino acid sequences SEQ ID NOs: 1577, 1578, or 1579. The term “GIPR polypeptide,” however, also encompasses polypeptides comprising an amino acid sequence that has been modified relative to the amino acid sequence of a naturally occurring GIPR polypeptide sequence, e.g., SEQ ID NOs: 1577, 1578, or 1579, by one or more amino acids, such that the sequence is at least 90% identical to SEQ ID NOs: 1577, 1578, or 1579. Such modifications include, but are not limited to, one or more amino acid substitutions, including substitutions with non-naturally occurring amino acids, non-naturally-occurring amino acid analogs, and amino acid mimetics. For example, GIPR polypeptides can be generated by introducing one or more amino acid substitutions, either conservative or non-conservative and using naturally or non-naturally occurring amino acids, at particular positions of the GIPR polypeptide.

In some embodiments, a GIPR polypeptide comprises an amino acid sequence that is at least 90 percent identical to a naturally-occurring GIPR polypeptide (e.g., SEQ ID NOs: 1577, 1578, or 1579). In some embodiments, a GIPR polypeptide comprises an amino acid sequence that is at least 95, at least 96, at least 97, at least 98, or at least 99 percent identical to a naturally-occurring GIPR polypeptide amino acid sequence (e.g., SEQ ID NOs: 1577, 1578, or 1579). Such GIPR polypeptides preferably, but need not, possess at least one activity of a wild-type GIPR polypeptide, such as the ability to bind GIP. The present disclosure also encompasses nucleic acid molecules encoding such GIPR polypeptide sequences.

As used herein, the term “GIPR activity assay” (also referred to as a “GIPR functional assay”) means an assay that can be used to measure GIP or a GIP binding protein activity in a cellular setting. In some embodiments, the “activity assay” or “functional assay” can be a cAMP assay in GIPR-expressing cells, in which GIP can induce cAMP signal, and the activity of a GIP/GIPR binding protein could be measured in the presence/absence of GIP ligand, in which IC₅₀/EC₅₀ and degree of inhibition/activation can be obtained (Biochemical and Biophysical Research Communications (2002) 290:1420-1426). In other embodiments, the “activity assay” or “functional assay” can be an insulin secretion assay in pancreatic beta cells, in which GIP can induce glucose-dependent insulin secretion, and the activity of a GIP/GIPR binding protein could be measured in the presence/absence of GIP ligand, in which IC₅₀/EC₅₀ and degree of inhibition/activation can be obtained (Biochemical and Biophysical Research Communications (2002) 290:1420-1426).

As used herein, the term “GIPR binding assay” refers to an assay that can be used to measure binding of GIP to GIPR. In some embodiments, a “GIPR binding assay” can be an assay using Fluorometric Microvolume Assay Technology (“FMAT”) or Fluorescence-Activated Cell Sorting (“FACS”) that measures fluorescence-labeled GIP binding to GIPR expression cells, and GIP/GIPR binding protein's activity can be measured for displacing fluorescence-labeled GIP binding to GIPR expression cells. In other embodiments, a “GIPR binding assay” can be an assay that measures radioactive-labeled GIP binding to GIPR expression cells, and GIP/GIPR binding protein's activity can be measured for displacing radioactive labeled GIP binding to GIPR expression cells (Biochimica et Biophysica Acta (2001) 1547:143-155).

As used herein, a “GIPR antagonist” refers to a molecule that reduces or inhibits GIP activation of GIPR. Such antagonists include chemically synthesized small molecules and antigen binding proteins. In some embodiments, a GIPR antagonist may reduce or inhibit GIP activation of GIPR by preventing binding of GIP to GIPR.

As used herein, the term “compete,” when used in the context of antigen-binding proteins (e.g., antibodies), means competition between antigen-binding proteins is determined by an assay in which the tested antigen-binding protein (e.g., antibody or immunologically functional fragment thereof) prevents or inhibits specific binding of a reference antigen-binding protein to a common antigen (e.g., GIPR or a fragment thereof). Numerous types of competitive binding assays can be used, for example: solid phase direct or indirect radioimmunoassay (RIA); solid phase direct or indirect enzyme immunoassay (EIA); sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619); solid phase direct labeled assay; solid phase direct labeled sandwich assay (see, e.g., Harlow and Lane, 1988, Antibodies, A Laboratory Manual, Cold Spring Harbor Press); solid phase direct label RIA using I-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antigen binding protein, and a labeled reference antigen binding protein. Competitive inhibition is measured by determining the amount of label bound to the solid surface or cells in the presence of the test antigen binding protein. Usually, the test antigen-binding protein is present in excess. Commonly, when a competing antigen binding protein is present in excess, it will inhibit specific binding of a reference antigen binding protein to a common antigen by at least 40%, at least 45%, at least 50%, at least 55%, at least 60%, at least 65%, at least 70%, or at least 75%. In some instances, binding is inhibited by at least 80%, at least 85%, at least 90%, at least 95%, or at least 97%.

As used herein, the term “encoding” refers to a polynucleotide sequence encoding one or more amino acids. The term does not require a start or stop codon.

As used herein, the term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. The modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate, and phosphoroamidate.

As used herein, the term “oligonucleotide” means a polynucleotide comprising 200 or fewer nucleotides. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including a radiolabel, a fluorescent label, a hapten or an antigenic label, for detection assays. Oligonucleotides may be used, for example, as PCR primers, cloning primers, or hybridization probes.

Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

As used herein, the term “control sequence” refers to a polynucleotide sequence that can affect the expression and processing of coding sequences to which it is ligated. The nature of such control sequences may depend upon the host organism. In some embodiments, control sequences for prokaryotes may include a promoter, a ribosomal binding site, and a transcription termination sequence. For example, control sequences for eukaryotes may include promoters comprising one or a plurality of recognition sites for transcription factors, transcription enhancer sequences, and transcription termination sequences. “Control sequences” can include leader sequences and/or fusion partner sequences.

As used herein, the term “isolated nucleic acid molecule” refers to a single- or double-stranded polymer of deoxyribonucleotide or ribonucleotide bases read from the 5′ to the 3′ end, or an analog thereof, that has been separated from at least about 50 percent of polypeptides, peptides, lipids, carbohydrates, polynucleotides, or other materials with which the nucleic acid is naturally found when total nucleic acid is isolated from the source cells. In some embodiments, an isolated nucleic acid molecule is substantially free from any other contaminating nucleic acid molecules or other molecules that are found in the natural environment of the nucleic acid that would interfere with its use in polypeptide production or its therapeutic, diagnostic, prophylactic, or research use.

Polypeptides described herein can be engineered and/or produced using standard molecular biology methodology. For example, a nucleic acid sequence encoding a GIPR, which can comprise all or a portion of SEQ ID NOs: 1577, 1578, or 1579, can be isolated and/or amplified from genomic DNA, or cDNA using appropriate oligonucleotide primers. Primers can be designed based on the nucleic and amino acid sequences provided herein according to standard (RT)-PCR amplification techniques. The amplified GIPR nucleic acid can then be cloned into a suitable vector and characterized by DNA sequence analysis.

Oligonucleotides for use as probes in isolating or amplifying all or a portion of the amino acid sequences provided herein can be designed and generated using standard synthetic techniques, e.g., automated DNA synthesis apparatus, or can be isolated from a longer sequence of DNA.

As used herein, the term “host cell” means a cell that has been transformed with a nucleic acid sequence and thereby expresses a gene of interest. The term includes the progeny of the parent cell, whether or not the progeny is identical in morphology or in genetic make-up to the original parent cell, so long as the gene of interest is present. In some embodiments, the host cell is a mammalian, non-human host cell. Representative host cells include, but are not limited to, those hosts typically used for cloning and expression, including Escherichia coli strains TOP10F′, TOP10, DH10B, DH5a, HB101, W3110, BL21(DE3) and BL21 (DE3)pLysS, BLUESCRIPT (Stratagene), mammalian cell lines CHO, CHO-K1, HEK293, 293-EBNA pIN vectors (Van Heeke & Schuster, J. Biol. Chem. 264: 5503-5509 (1989); pET vectors (Novagen, Madison Wis.).

In some embodiments, host cells comprising vectors disclosed herein are provided. In some embodiments, a vector or nucleic acid is integrated into the host cell genome; in other embodiments, the vector is extra-chromosomal.

As used herein, the term “vector” means any molecule or entity (e.g., nucleic acid, plasmid, bacteriophage, or virus) used to transfer protein coding information into a host cell. A “vector” refers to a delivery vehicle that (a) promotes the expression of a polypeptide-encoding nucleic acid sequence; (b) promotes the production of the polypeptide therefrom; (c) promotes the transfection/transformation of target cells therewith; (d) promotes the replication of the nucleic acid sequence; (e) promotes stability of the nucleic acid; (f) promotes detection of the nucleic acid and/or transformed/transfected cells; and/or (g) otherwise imparts advantageous biological and/or physiochemical function to the polypeptide-encoding nucleic acid. A vector can be any suitable molecule or entity, including chromosomal, non-chromosomal, and synthetic nucleic acid vectors (a nucleic acid sequence comprising a suitable set of expression control elements). Non-limiting examples of vectors include derivatives of SV40, bacterial plasmids, phage DNA, baculovirus, yeast plasmids, vectors derived from combinations of plasmids and phage DNA, and viral nucleic acid (RNA or DNA) vectors.

As used herein, the term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct may include, but is not limited to, sequences that affect or control transcription, translation, and, if introns are present, affect RNA splicing of a coding region operably linked thereto.

In order to express a polypeptide provided herein, the appropriate coding sequence(s) can be cloned into a suitable vector and after introduction in a suitable host, the sequence can be expressed to produce the encoded polypeptide according to standard cloning and expression techniques, which are known in the art (e.g., as described in Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989). In some embodiments, the present disclosure provides such vectors comprising a nucleic acid sequence encoding an amino acid sequence described herein.

A recombinant expression vector can be designed for expression of a protein in prokaryotic (e.g., E. coli) or eukaryotic cells (e.g., insect cells, using baculovirus expression vectors, yeast cells, or mammalian cells). Alternatively, a recombinant expression vector can be transcribed and translated in vitro, for example, using T7 promoter regulatory sequences and T7 polymerase and an in vitro translation system. In some embodiments, the vector contains a promoter upstream of the cloning site containing the nucleic acid sequence encoding the polypeptide. Examples of promoters, which can be switched on and off, include, but are not limited to, the lac promoter, the T7 promoter, the trc promoter, the tac promoter, and the trp promoter.

A vector can comprise or be associated with any suitable promoter, enhancer, and other expression-facilitating elements. Examples of such elements include strong expression promoters (e.g., a human CMV IE promoter/enhancer, an RSV promoter, SV40 promoter, SL3-3 promoter, MMTV promoter, or HIV LTR promoter, EF1alpha promoter, CAG promoter), effective poly (A) termination sequences, an origin of replication for plasmid product in E. coli, an antibiotic resistance gene as a selectable marker, and/or a convenient cloning site (e.g., a polylinker). Vectors also can comprise an inducible promoter as opposed to a constitutive promoter such as CMV IE. In some embodiments, a nucleic acid comprising an amino acid sequence described herein which is operably linked to a tissue-specific promoter which promotes expression of the sequence in a metabolically-relevant tissue, such as liver or pancreatic tissue, is provided.

In some embodiments, a nucleic acid can be positioned in and/or delivered to a host cell or host animal via a viral vector. Any suitable viral vector can be used in this capacity. A viral vector can comprise any number of viral polynucleotides, alone or in combination with one or more viral proteins, which facilitate delivery, replication, and/or expression of the nucleic acid of the present disclosure in a desired host cell. The viral vector can be a polynucleotide comprising all or part of a viral genome, a viral protein/nucleic acid conjugate, a virus-like particle (VLP), or an intact virus particle comprising viral nucleic acids and a GIPR polypeptide-encoding nucleic acid. A viral particle viral vector can comprise a wild-type viral particle or a modified viral particle. The viral vector can be a vector which requires the presence of another vector or wild-type virus for replication and/or expression (e.g., a viral vector can be a helper-dependent virus), such as an adenoviral vector amplicon. Typically, such viral vectors consist of a wild-type viral particle, or a viral particle modified in its protein and/or nucleic acid content to increase transgene capacity or aid in transfection and/or expression of the nucleic acid (examples of such vectors include the herpes virus/AAV amplicons). Typically, a viral vector is similar to and/or derived from a virus that normally infects humans. Suitable viral vector particles in this respect, include, but are not limited to, adenoviral vector particles (including any virus of or derived from a virus of the adenoviridae), adeno-associated viral vector particles (AAV vector particles) or other parvoviruses and parvoviral vector particles, papillomaviral vector particles, flaviviral vectors, alphaviral vectors, herpes viral vectors, pox virus vectors, retroviral vectors, including lentiviral vectors.

As used herein, “operably linked” means that the components to which the term is applied are in a relationship that allows them to carry out their inherent functions under suitable conditions. For example, a control sequence in a vector that is “operably linked” to a protein coding sequence is ligated thereto so that expression of the protein coding sequence is achieved under conditions compatible with the transcriptional activity of the control sequences.

As used herein, the terms “glucagon agonist” and “glucagon receptor (GCGR) agonist” are used interchangeably and refer to a molecule that mimics a biological activity of a glucagon molecule with respect to a glucagon receptor.

As used herein, the term “glucagon analog” refers to a molecule which elicits a biological activity similar to that of glucagon, when evaluated by art-known measures such as receptor binding assays or in vivo blood glucose assays as described, e.g., by Hargrove et al., Regulatory Peptides, 141:113-119 (2007), the disclosure of which is incorporated by reference herein. In some embodiments, the term “glucagon analog” refers to a peptide that has an amino acid sequence with 1, 2, 3, 4, 5, 6, 7 or 8 amino acid substitutions, insertions, deletions, or a combination of two or more of the preceding, when compared to the amino acid sequence of a glucagon. In some embodiments, the glucagon analog is glucagon-NH2. Glucagon analogs include the amidated forms, the acid form, the pharmaceutically acceptable salt form, and any other physiologically active form of the molecule. In some embodiments, a simple nomenclature is used to describe the glucagon receptor agonist, e.g., “glucagon (s2)” glucagon” or “glucagon S2s” designates an analog of glucagon wherein the naturally occurring L-serine at position 2 has been substituted with D-serine.

As used herein, the terms “GLP-1 agonist” and “GLP-1R agonist” are used interchangeably and refer to a molecule that mimics a biological activity of a GLP-1 molecule with respect to a GLP-1R.

As used herein, a “GIPR antagonist” refers to a molecule that partially or fully blocks, inhibits, or neutralizes a biological activity of a GIPR molecule.

As used herein, the term “pharmaceutically acceptable” refers to a species or component that is generally safe, non-toxic, and neither biologically nor otherwise undesirable for use in a subject.

As used herein, the term “pharmaceutically acceptable excipient” refers to a broad range of ingredients that may be combined with a polypeptide or molecule disclosed herein to prepare a pharmaceutically acceptable composition or formulation. Excipients include, for example, vehicles (e.g., solvents, dispersion media), coatings, isotonic and absorption delaying agents, diluents, colorants, glidants, disintegrants, flavoring agents, coatings, binders, sweeteners, lubricants, sorbents, and preservatives (e.g., antibacterial and antifungal agents).

As used herein, “substantially pure” means that the described species is the predominant species present, that is, on a molar basis it is more abundant than any other individual species in the same mixture. In some embodiments, a substantially pure molecule is a composition wherein the object species comprises at least 50% (on a molar basis) of all macromolecular species present. In other embodiments, a substantially pure composition will comprise at least 80%, at least 85%, at least 90%, at least 95%, at least 96%, at least 97%, at least 98%, or at least 99% of all macromolecular species present in the composition. In other embodiments, the object species is purified to essential homogeneity wherein contaminating species cannot be detected in the composition by conventional detection methods and thus the composition consists of a single detectable macromolecular species.

As used herein, the term “treating” refers to any indicia of success in the treatment or amelioration of an injury, pathology, or condition, including any objective or subjective parameter, such as, e.g., abatement, remission, diminishing of symptoms or making the injury, pathology, or condition more tolerable to the patient, slowing in the rate of degeneration or decline, making the final point of degeneration less debilitating, or improving a patient's physical or mental well-being. The treatment or amelioration of symptoms can be based on objective or subjective parameters, including, e.g., the results of a physical examination, neuropsychiatric exam, or a psychiatric evaluation.

As used herein, the term “therapeutically effective amount” refers to that amount of a polypeptide or molecule disclosed herein that elicits a desired biological or medical response in a cell, a tissue, a system, or a subject. The desired biological or medical response does not necessarily occur by administration of one dose, and may occur only after administration of a series of doses. Thus, a therapeutically effective amount may be administered in one or more administrations.

As used herein, the term “patient” or “subject” refers to humans and other mammals. The term “mammal” as used herein includes, for example, humans, non-human primates, cattle, sheep, goats, pigs, horses, cats, dog, rabbits, rodents (e.g., rats or mice), and monkeys. Human subjects include neonates, infants, juveniles, adults, and geriatric subjects. In some embodiments, the subject or patient is a human. In some embodiments, the subject or patient is an adult human.

### Glucagon Receptor Agonists

The present disclosure provides polypeptides that agonize a glucagon receptor (“GCGR”). Such polypeptides may be referred to as polypeptide agonists, glucagon receptor agonists, or GCGR agonists herein. The polypeptide agonists provided herein are glucagon analogs that mimic at least one biological activity of a glucagon molecule (SEQ ID NO: 1576) with respect to a glucagon receptor. Relative to native glucagon, such polypeptide agonists may possess one or more advantageous properties. For example, in some embodiments, a polypeptide agonist may exhibit improved stability relative to native glucagon.

Non-limiting examples of polypeptide agonists of the present disclosure are presented in Table 2A and Table 2B. Descriptions associated with the sequences are non-limiting and provided for the purpose of illustration, e.g., the N- and C-termini of the example sequences of Table 2A and Table 2B may be modified as described herein without being limited by the descriptions (e.g., OH; NH₂) provided. Illustratively, in some non-limiting embodiments, the C-termini of the disclosed sequences may be unmodified (e.g., terminating with an —OH), amidated (terminating with an —NH₂), or connected to another polypeptide sequence via, for example, an amide bond.

The following abbreviations for non-canonical amino acids are used in the Tables below.
Aad: L-α-aminoadipic acid
Aib: 2-aminoisobutyric acid
Azk: 6-azido-L-lysine
hPhe: homophenylalanine
2-Nal: 2-naphthylalanine
Bip: L-4,4′-biphenylalanine
Cit: citrulline
4-CiF: 4-chloro-L-phenylalanine
hSer: homoserine
Cha: β-cyclohexyl-L-alanine
Dpr: 2,3-diaminopropionic acid
5-BrW: 5-bromo-L-tryptophan
BhTrp: L-beta-homotryptophan
5-MeOW: 5-methoxy-L-tryptophan
5-MeW: 5-methyl-L-tryptophan
6-BrW: 6-bromo-L-tryptophan
6-ClW: 6-chloro-L-tryptophan
6-MeW: 6-methyl-L-tryptophan
7-MeW: 7-methyl-L-tryptophan

TABLE 2A
Examples of GCG Receptor Agonist Sequences

| Description | Sequence | SEQ ID NO: |
|---|---|---|
| [s2, Aib16, K17, D24, 5- | HsQGT FTSDY SKYLD [Aib]KRAQ | 1587 |
| BrW25, L27, K28]-GCG-OH | DFVD[5-BrW] LLKT | |
| [s2, Aib16, 5- | HsQGT FTSDY SKYLD [Aib ]RRAQ | 1588 |
| BrW25, L27, K28]-GCG-OH | DFVQ[5-BrW] LLKT | |
| [s2, Aib16, K24, 5- | HsQGT FTSDY SKYLD [Aib]RRAQ | 1589 |
| BrW25, L27, D28]-GCG-OH | DFVK[5-BrW] LLDT | |
| [s2, Aib16, K17, Ala24, 5- | HsQGT FTSDY SKYLD [Aib]KRAQ | 1590 |
| BrW25, L27, K28]-GCG-OH | DFVA[5-BrW] LLKT | |
| [s2, Aib16, A24, 5- | HsQGT FTSDY SKYLD [Aib]RRAQ | 1591 |
| BrW25, K28]-GCG-OH | DFVA[5-BrW] LLKT | |
| [s2, Aib16, K17, K24, 5- | HsQGT FTSDY SKYLD [Aib]KRAQ | 1592 |
| BrW25, L27, D28]-GCG-OH | DFVK[5-BrW] LLDT | |
| [s2, Aib16, K17, E24, 5- | HsQGT FTSDY SKYLD [Aib]KRAQ | 1593 |
| BrW25, L27, K28]-GCG-OH | DFVE[5-BrW] LLKT | |
| [s2, Aib16, 2-Nal18, K24, 5- | HsQGT FTSDY SKYLD [Aib]R[2-Nal] | 1594 |
| BrW25, L27, D28]-GCG-OH | AQDFVK[5-BrW] LLDT | |
| [s2, Aib16, 2-Nal18, Ala24, | HsQGT FTSDY SKYLD [Aib]R[2-Nal] | 1595 |
| 5-BrW25, L27, K28]-GCG-OH | AQDFVA[5-BrW] LLKT | |
| [s2, Aib16, L27, K28]-GCG- | HsQGT FTSDY SKYLD [Aib]RRAQ | 1596 |
| OH | DFVQW LLKT | |
| [s2, Aib16, K24, L27, D28]- | HsQGT FTSDY SKYLD [Aib]RRAQ | 1597 |
| GCG-OH | DFVKW LLDT | |
| [s2, Aib16, K24, E27, S28]- | HsQGT FTSDY SKYLD [Aib]RRAQ | 1598 |
| GCG-OH | DFVKW LEST | |
| [s2, Aib16, K17, L27, K28]- | HsQGT FTSDY SKYLD [Aib]KRAQ | 1599 |
| GCG-OH | DFVQW LLKT | |
| [Y1, s2, Aib16, L27, K28]- | YsQGT FTSDY SKYLD [Aib]RRAQ | 1600 |
| GCG-OH | DFVQW LLKT | |
| [Y1, Aib2, Aib16, L27, K28]- | Y[Aib]QGT FTSDY SKYLD [Aib]RRAQ | 1601 |
| GCG-OH | DFVQW LLKT | |
| [t2, Aib16, L27, K28]-GCG- | HtQGT FTSDY SKYLD [Aib]RRAQ | 1602 |
| OH | DFVQW LLKT | |
| [s2, Aib16, A24, L27, K28]- | HsQGT FTSDY SKYLD [Aib]RRAQ | 1603 |
| GCG-OH | DFVAW LLKT | |
| [s2, Aib16, Aib24, L27, K28]- | HsQGT FTSDY SKYLD [Aib]RRAQ | 1604 |
| GCG-OH | DFV[Aib]W LLKT | |
| [s2, Aib16, Aib24, E27, K28]- | HsQGT FTSDY SKYLD [Aib]RRAQ | 1605 |
| GCG-OH | DFV[Aib]W LEKT | |
| [s2, Aib16, Y25, L27, K28]- | HsQGT FTSDY SKYLD [Aib]RRAQ | 1606 |
| GCG-OH | DFVQY LLKT | |
| [s2, Aib16, Bip18, L27, K28]- | HsQGT FTSDY SKYLD [Aib]R[Bip]AQ | 1607 |
| GCG-OH | DFVQW LLKT | |
| [s2, Aib16, 2-NaI18, L27, K28]- | HsQGT FTSDY SKYLD [Aib]R[2-Nal] | 1608 |
| GCG-OH | AQDFVQW LLKT | |
| [s2, Aib16, Cit17, L27, K28]- | HsQGT FTSDY SKYLD [Aib][Cit]RAQ | 1609 |
| GCG-OH | DFVQW LLKT | |
| [Aib16, Aad27, K28]-GCG- | HSQGT FTSDY SKYLD [Aib]RRAQ | 1610 |
| OH | DFVQW L[Aad]KT | |
| [s2, Aib16, Q17, L27, K28]- | HsQGT FTSDY SKYLD [Aib]QRAQ | 1611 |
| GCG-OH | DFVQW LLKT | |
| [s2, H7, Aib16, L27, K28, E29]- | HsQGT FHSDY SKYLD [Aib]RRAQ | 1612 |
| GCG-OH | DFVQW LLKE | |
| [s2, Aib16, K24, L27, D28, S29] | HsQGT FTSDY SKYLD [Aib]RRAQ | 1613 |
| -GCG-OH | DFVKW LLDS | |
| [Aib16, G24, L27, K28]-GCG- | HSQGT FTSDY SKYLD [Aib]RRAQ | 1614 |
| OH | DFVGW LLKT | |
| [s2, Aib16, D24, L27, K28]- | HsQGT FTSDY SKYLD [Aib]RRAQ | 1615 |
| GCG-OH | DFVDW LLKT | |
| [s2, Aib16, D24, L27, K28, E29] | HsQGT FTSDY SKYLD [Aib]RRAQ | 1616 |
| -GCG-OH | DFVDW LLKE | |
| [s2, Aib16, H24, L27, K28]- | HsQGT FTSDY SKYLD [Aib]RRAQ | 1617 |
| GCG-OH | DFVHW LLKT | |
| [s2, Aib16, K24, L27, K28]- | HsQGT FTSDY SKYLD [Aib]RRAQ | 1618 |
| GCG-OH | DFVKW LLKT | |
| [s2, Aib16, N24, L27, K28]- | HsQGT FTSDY SKYLD [Aib]RRAQ | 1619 |
| GCG-OH | DFVNW LLKT | |
| [s2, Aib16, T24, L27, K28]- | HsQGT FTSDY SKYLD [Aib]RRAQ | 1620 |
| GCG-OH | DFVTW LLKT | |
| [s2, Aib16, E24, L27, K28]- | HsQGT FTSDY SKYLD [Aib]RRAQ | 1621 |
| GCG-OH | DFVEW LLKT | |
| [s2, Aib16, L27, K28, D29]- | HsQGT FTSDY SKYLD [Aib]RRAQ | 1622 |
| GCG-OH | DFVQW LLKD | |
| [s2, Aib16, L27, K28, A29]- | HsQGT FTSDY SKYLD [Aib]RRAQ | 1623 |
| GCG-OH | DFVQW LLKA | |
| [s2, Aib16, BhTrp25, L27, K28] | HsQGT FTSDY SKYLD [Aib]RRAQ | 1624 |
| -GCG-OH | DFVQ[BhTrp] LLKT | |
| [Aib16, K17, K24, L27, D28]- | HSQGT FTSDY SKYLD [Aib]KRAQ | 1625 |
| GCG-OH | DFVKW LLDT | |
| [s2, Aib16, K17, D24, L27, K28] | HsQGT FTSDY SKYLD [Aib]KRAQ | 1626 |
| -GCG-OH | DFVDW LLKT | |
| [s2, Aib16, K17, E24, L27, K28] | HsQGT FTSDY SKYLD [Aib]KRAQ | 1627 |
| -GCG-OH | DFVEW LLKT | |
| [s2, Aib16, q24, L27, K28]- | HsQGT FTSDY SKYLD [Aib]RRAQ | 1747 |
| GCG-OH | DFVqW LLKT | |

TABLE 2B
Additional Examples of GCG Receptor Agonist Sequences

| Description | Sequence | SEQ ID NO: |
|---|---|---|
| [s2, Aib16, A17, L27, K28]- | HsQGTFTSDYSKYLD[Aib]ARAQDFVQ | 1748 |
| GCG-OH | WLLKT | |
| [Aib16, L27, K28]-GCG-OH | HSQGTFTSDYSKYLD[Aib]RRAQDFVQ | 1749 |
| | WLLKT | |
| [s2, Aib16, L27, Azk28]-GCG- | HsQGTFTSDYSKYLD[Aib]RRAQDFVQ | 1750 |
| OH | WLL[AzK]T | |
| [Y1, Aib2, L27, K28]-GCG-OH | Y[Aib]QGTFTSDYSKYLDSRRAQDFVQ | 1751 |
| | WLLKT | |
| [A16, K17, E21, E24, L27, K28, | HSQGTFTSDYSKYLDAKRAQEFVEWL | 1752 |
| S29]-GCG-OH | LKS | |
| [Aib16, E24, 5- | HSQGTFTSDYSKYLD[Aib]RRAQDFVE[ | 1753 |
| BrW25, L27, K28]-GCG-OH | 5-BrWILLKT | |
| [F1, s2, Aib16, L27, K28]- | FsQGTFTSDYSKYLD[Aib]RRAQDFVQ | 1754 |
| GCG-OH | WLLKT | |
| [s2, Aib16, 4-CIF20, L27, K28]- | HsQGTFTSDYSKYLD[Aib]RRA[4-C1- | 1755 |
| GCG-OH | F]DFVQWLLKT | |
| [s2, Aib16, 5- | HsQGTFTSDYSKYLD[Aib]RRAQDFVQ[ | 1756 |
| MeOW25, L27, K28]-GCG- | 5-MeOW]LLKT | |
| OH | | |
| [s2, Aib 16, 5- | HsQGTFTSDYSKYLD[Aib]RRAQDFVQ[ | 1757 |
| MeW25, L27, K28]-GCG-OH | 5-MeWILLKT | |
| [s2, Aib16, 6- | HsQGTFTSDYSKYLD[Aib]RRAQDFVQ[ | 1758 |
| BrW25, L27, K28]-GCG-OH | 6-BrWILLKT | |
| [s2, Aib16, 6- | HsQGTFTSDYSKYLD[Aib]RRAQDFVQ[ | 1759 |
| CIW25, L27, K28]-GCG-OH | 6-CIWILLKT | |
| [s2, Aib16, 6- | HsQGTFTSDYSKYLD[Aib]RRAQDFVQ[ | 1760 |
| MeW25, L27, K28]-GCG-OH | 6-MeWILLKT | |
| [s2, Aib16, 7- | HsQGTFTSDYSKYLD[Aib]RRAQDFVQ[ | 1761 |
| MeW25, L27, K28]-GCG-OH | 7-MeWILLKT | |
| [s2, Aib16, A18, K24, E27, D28, | HsQGTFTSDYSKYLD[Aib]RAAQDFVK | 1762 |
| S29]-GCG-NH2 | WLEDS | |
| [s2, Aib16, A20, A24, L27, K28] | HsQGTFTSDYSKYLD[Aib]RRAADFVA | 1763 |
| -GCG-OH | WLLKT | |
| [s2, Aib16, E27, K28]-GCG- | HsQGTFTSDYSKYLD[Aib]RRAQDFVQ | 1764 |
| OH | WLEKT | |
| [s2, Aib16, Q17, E27, K28]- | HsQGTFTSDYSKYLD[Aib]QRAQDFVQ | 1765 |
| GCG-OH | WLEKT | |
| [s2, Aib16, W22, L27, K28]- | HsQGTFTSDYSKYLD[Aib]RRAQDWVQ | 1766 |
| GCG-OH | WLLKT | |
| [Y1, s2, Aib16, K24, E27, D28, S | YsQGTFTSDYSKYLD[Aib]RRAQDFVK | 1767 |
| 29]-GCG-OH | WLEDS | |
| [Y1, s2, Aib16, Q17, K24, E27, | YsQGTFTSDYSKYLD[Aib]QRAQDFVK | 1768 |
| D28, S29]-GCG-OH | WLEDS | |
| [s2, Aib16, Cit21, L27, K28]- | HsQGTFTSDYSKYLD[Aib]RRAQ[Cit] | 1769 |
| GCG-OH | FVQWLLKT | |
| [s2, Aib16, L27, K28, E29]- | HsQGTFTSDYSKYLD[Aib]RRAQDFVQ | 1770 |
| GCG-OH | WLLKE | |
| [s2, Aib16, Cit18, L27, K28]- | HsQGTFTSDYSKYLD[Aib]R[Cit] | 1771 |
| GCG-OH | AQDFVQWLLKT | |
| [s2, Aib16, q20, L27, K28]- | HsQGTFTSDYSKYLD[Aib]RRAqDFVQ | 1772 |
| GCG-OH | WLLKT | |
| [s2, Aib16, d21, L27, K28]- | HsQGTFTSDYSKYLD[Aib]RRAQdFVQ | 1773 |
| GCG-OH | WLLKT | |
| [s2, H7, Aib16, L27, K28]- | HsQGTFHSDYSKYLD[Aib]RRAQDFVQ | 1774 |
| GCG-OH | WLLKT | |
| [s2, Aib16, R24, L27, K28]- | HsQGTFTSDYSKYLD[Aib]RRAQDFVR | 1775 |
| GCG-OH | WLLKT | |
| [s2, Aib16, F24, L27, K28]- | HsQGTFTSDYSKYLD[Aib]RRAQDFVF | 1776 |
| GCG-OH | WLLKT | |
| [s2, Aib16, L24, L27, K28]- | HsQGTFTSDYSKYLD[Aib]RRAQDFVL | 1777 |
| GCG-OH | WLLKT | |
| [s2, Aib16, S24, L27, K28]- | HsQGTFTSDYSKYLD[Aib]RRAQDFVS | 1778 |
| GCG-OH | WLLKT | |
| [s2, Aib16, Y24, L27, K28]- | HsQGTFTSDYSKYLD[Aib]RRAQDFVY | 1779 |
| GCG-OH | WLLKT | |
| [s2, Aib16, V24, L27, K28]- | HsQGTFTSDYSKYLD[Aib]RRAQDFVV | 1780 |
| GCG-OH | WLLKT | |
| [s2, Aib16, 124, L27, K28]- | HsQGTFTSDYSKYLD[Aib]RRAQDFVI | 1781 |
| GCG-OH | WLLKT | |
| [s2, Aib16, hSer24, L27, K28]- | HsQGTFTSDYSKYLD[Aib]RRAQDFV[h | 1782 |
| GCG-OH | Ser]WLLKT | |
| [s2, Aib16, Dpr24, L27, K28]- | HsQGTFTSDYSKYLD[Aib]RRAQDFV[D | 1783 |
| GCG-OH | pr]WLLKT | |
| [s2, Q16, L27, K28]-GCG-OH | HsQGTFTSDYSKYLDQRRAQDFVQWL | 1784 |
| | LKT | |
| [s2, Aib16, hSer20, L27, K28]- | HsQGTFTSDYSKYLD[Aib]RRA[hSer]DF | 1785 |
| GCG-OH | VQWLLKT | |
| [s2, Aib16, K18, E21, L27, K28] | HsQGTFTSDYSKYLD[Aib]RKAQEFVQ | 1786 |
| -GCG-OH | WLLKT | |
| [s2, Aib16, K24, E27, S28, E29]- | HsQGTFTSDYSKYLD[Aib]RRAQDFVK | 1787 |
| GCG-OH | WLESE | |
| [s2, Aib16, K24, L27, D28, E29] | HsQGTFTSDYSKYLD[Aib]RRAQDFVK | 1788 |
| -GCG-NH2 | WLLDE | |
| [s2, Aib16, K24, L27, D28, E29] | | |
| -GCG-OH | | |
| [s2, Aib16, L27, D28, K30, K31] | HsQGTFTSDYSKYLD[Aib]RRAQDFVQ | 1789 |
| -GCG-NH2 | WLLDTKK | |
| [s2, Aib16, Q17, A24, E27, 29, K | HsQGTFTSDYSKYLD[Aib]QRAQDFVA | 1790 |
| 28]-GCG-OH | WLEKE | |
| [s2, F7, Aib16, L27, K28, E29]- | HsQGTFFSDYSKYLD[Aib]RRAQDFVQ | 1791 |
| GCG-OH | WLLKE | |
| [s2, hPhe16, L27, K28]-GCG- | HsQGTFTSDYSKYLD[hPhe]RRAQDFVQ | 1792 |
| OH | WLLKT | |
| [s2, Aib16, E24, L27, D28, S29]- | HsQGTFTSDYSKYLD[Aib]RRAQDFVE | 1793 |
| GCG | WLLDS | |
| [s2, Aib16, Cha22, L27, K28]- | HsQGTFTSDYSKYLD[Aib]RRAQD[Cha] | 1794 |
| GCG-OH | VQWLLKT | |
| [s2, W10, Aib16, L27, K28]- | HsQGTFTSDWSKYLD[Aib]RRAQDFVQ | 1795 |
| GCG-OH | WLLKT | |
| [s2, Aib16, A24, E27, E29, K28] | HsQGTFTSDYSKYLD[Aib]RRAQDFVA | 1796 |
| -GCG-OH | WLEKE | |
| [s2, Aib16, K24, E27, D28, S29] | HsQGTFTSDYSKYLD[Aib]RRAQDFVK | 1797 |
| -GCG-OH | WLEDS | |
| [s2, Aib16, Nal18, K24, L27, D2 | HsQGTFTSDYSKYLD[Aib]R[2- | 1798 |
| 8]-GCG-OH | Nal]AQDFVKWLLDT | |
| [s2, Aib16, A24, L27, D28, S29] | HsQGTFTSDYSKYLD[Aib]RRAQDFVA | 1799 |
| -GCG | WLLDS | |
| [s2, Aib16, A24, L27, D28, E29] | HsQGTFTSDYSKYLD[Aib]RRAQDFVA | 1800 |
| -GCG | WLLDE | |
| [s2, Aib16, K17, A24, L27, K28, | HsQGTFTSDYSKYLD[Aib]KRAQDFVA | 1801 |
| E29]-GCG-OH | WLLKE | |
| [s2, Aib16, A24, L27, K28, E29] | HsQGTFTSDYSKYLD[Aib]RRAQDFVA | 1802 |
| -GCG-OH | WLLKE | |
| [s2, Aib16, D24, E27, E29, K28] | HsQGTFTSDYSKYLD[Aib]RRAQDFVD | 1803 |
| -GCG-OH | WLEKE | |
| [s2, Aib16, H20, K24, L27]- | HsQGTFTSDYSKYLD[Aib]RRAHDFVK | 1804 |
| GCG-NH2 | WLLNT | |
| [s2, Aib16, K24, L27, D28, S29] | HsQGTFTSDYSKYLD[Aib]RRAQDFVK | 1805 |
| -GCG-NH2 | WLLDS | |
| [s2, Aib16, D24, L27, K28, S29] | HsQGTFTSDYSKYLD[Aib]RRAQDFVD | 1806 |
| GCG-OH | WLLKS | |
| [s2, Aib16, E24, L27, K28, N29] | HsQGTFTSDYSKYLD[Aib]RRAQDFVE | 1807 |
| -GCG-OH | WLLKN | |
| [s2, Aib16, D24, 5- | HsQGTFTSDYSKYLD[Aib ]RRAQDFVD[ | 1808 |
| BrW25, L27, K28]-GCG-OH | 5-BrWILLKT | |
| [s2, Aib16, A24, 5- | HsQGTFTSDYSKYLD[Aib]RRAQDFVA[ | 1809 |
| BrW25, L27, K28, E29]-GCG- | 5-BrW]LLKE | |
| OH | | |
| [s2, Aib16, A24, 5- | HsQGTFTSDYSKYLD[Aib]RRAQDFVA[ | 1810 |
| BrW25, L27, K28, S29]-GCG- | 5-BrWILLKS | |
| OH | | |
| [s2, Aib16, K24, 5- | HsQGTFTSDYSKYLD[Aib]RRAQDFVK[ | 1811 |
| BrW25, L27, D28, E29]-GCG- | 5-BrWILLDE | |
| OH | | |
| [s2, Aib16, K24, 5- | HsQGTFTSDYSKYLD[Aib]RRAQDFVK[ | 1812 |
| BrW25, L27, D28, S29]-GCG- | 5-BrWILLDS | |
| OH | | |
| [s2, H7, Aib16, A24, 5- | HsQGTFHSDYSKYLD[Aib]RRAQDFVA[ | 1813 |
| BrW25, L27, K28]-GCG-OH | 5-BrWILLKT | |
| [s2, H7, Aib 16, A24, 5- | HsQGTFHSDYSKYLD[Aib]RRAQDFVA[ | 1814 |
| BrW25, L27, K28, S29]-GCG- | 5-BrWILLKS | |
| OH | | |
| [s2, Aib16, D24, 5- | HsQGTFTSDYSKYLD[Aib]RRAQDFVD[ | 1815 |
| BrW25, L27, K28, S29]-GCG- | 5-BrWILLKS | |
| OH | | |
| [s2, Aib16, E24, 5- | HsQGTFTSDYSKYLD[Aib]RRAQDFVE[ | 1816 |
| BrW25, L27, K28]-GCG-OH | 5-BrWILLKT | |
| [s2, Aib16, E24, 5- | HsQGTFTSDYSKYLD[Aib]RRAQDFVE[ | 1817 |
| BrW25, L27, K28, S29]-GCG- | 5-BrWILLKS | |
| OH | | |
| [s2, Aib16, K17, E24, L27, K28, | HsQGTFTSDYSKYLD[Aib]KRAQDFVE | 1818 |
| S29]-GCG-OH | WLLKS | |
| [s2, Aib16, K17, E24, L27, K28, | HsQGTFTSDYSKYLD[Aib]KRAQDFVE | 1819 |
| D29]-GCG-OH | WLLKD | |
| [s2, Aib16, K17, E24, E27, K28] | HsQGTFTSDYSKYLD[Aib]KRAQDFVE | 1820 |
| -GCG-OH | WLEKT | |
| [s2, Aib16, K17, E21, K24, L27, | HsQGTFTSDYSKYLD[Aib]KRAQEFVK | 1821 |
| E28]-GCG-OH | WLLET | |
| [s2, Aib16, K17, E21, K24, L27, | HsQGTFTSDYSKYLD[Aib]KRAQEFVK | 1822 |
| E28, S29]-GCG-OH | WLLES | |
| [s2, Aib16, K17, E24, L27, K28, | HsQGTFTSDYSKYLD[Aib]KRAQDFVE | 1823 |
| E29]-GCG-OH | WLLKE | |
| [s2, Aib16, K17, R20, E24, L27, | HsQGTFTSDYSKYLD[Aib]KRARDFVE | 1824 |
| K28, E29]-GCG-OH | WLLKE | |
| [s2, Aib16, K17, E21, E24, L27, | HsQGTFTSDYSKYLD[Aib]KRAQEFVE | 1825 |
| K28, S29]-GCG-OH | WLLKS | |
| [s2, Aib16, K17, E21, E24, L27, | HsQGTFTSDYSKYLD[Aib]KRAQEFVE | 1826 |
| K28, D29]-GCG-OH | WLLKD | |
| [s2, Aib16, K17, E21, E24, L27, | HsQGTFTSDYSKYLD[Aib]KRAQEFVE | 1827 |
| K28]-GCG(1-29)-OH | WLLKT | |
| [s2, Aib16, K17, R20, E21, E24, | HsQGTFTSDYSKYLD[Aib]KRAREFVE | 1828 |
| L27, K28, S29]-GCG(1-29)- | WLLKS | |
| OH | | |
| [s2, E16, K17, E21, E24, L27, K2 | HsQGTFTSDYSKYLDEKRAQEFVEWLL | 1829 |
| 8, S29]-GCG(1-29)-OH | KS | |
| [s2, Aib16, K17, E21, E24, L27, | HsQGTFTSDYSKYLD[Aib]KRAQEFVE | 1830 |
| K28, E29]-GCG(1-29)-NH2 | WLLKE | |
| [s2, Aib16, K17, E21, E24, L27, | HsQGTFTSDYSKYLD[Aib]KRAQEFVE | 1831 |
| E28, S29]-GCG(1-29) | WLLES | |
| [s2, Aib16, K17, E21, E24, L27, | HsQGTFTSDYSKYLD[Aib]KRAQEFVE | 1832 |
| E28, E29]-GCG(1-29) | WLLEE | |
| [s2, E15, Aib16, K17, E21, K24, | HsQGTFTSDYSKYLE[Aib]KRAQEFVK | 1833 |
| L27, E28, S29]-GCG(1-29)- | WLLES | |
| OH | | |
| [s2, E16, K17, R20, E21, E24, L2 | HsQGTFTSDYSKYLDEKRAREFVEWLL | 1834 |
| 7, K28, S29]-GCG(1-29)-OH | KS | |
| [s2, E3, Aib16, K17, E21, E24, L | HsEGTFTSDYSKYLD[Aib]KRAQEFVE | 1835 |
| 27, K28, S29]-GCG(1-29)-OH | WLLKS | |
| [s2, Aib16, K17, A18, E21, E24, | HsQGTFTSDYSKYLD[Aib]KAAQEFVE | 1836 |
| L27, K28, S29]-GCG(1-29)- | WLLKS | |
| OH | | |
| [s2, Aib16, K17, E20, E21, K24, | HsQGTFTSDYSKYLD[Aib]KRAEEFVK | 1837 |
| L27, E28, S29]-GCG(1-29)- | WLLES | |
| OH | | |
| [s2, Aib16, K17, E21, K24, L27, | HsQGTFTSDYSKYLD[Aib]KRAQEFVK | 1838 |
| A28, S29]-GCG(1-29)-OH | WLLAS | |
| [s2, E15, Aib16, K17, E21, E24, | HsQGT FTSDY SKYLE [Aib]KRAQ | 1839 |
| L27, K28, S29]-GCG(1-29)- | EFVEW LLKS | |
| OH | | |
| [s2, Aib16, a24, L27, K28]- | HsQGTFTSDYSKYLD[Aib]RRAQDFVa | 1840 |
| GCG-OH | WLLKT | |
| [K28]-GCG(1-29) | HSQGTFTSDYSKYLDSRRAQDFVKWL | 1859 |
| | MKT | |
| [K21]-GCG(1-29) | HSQGTFTSDYSKYLDSRRAQKFVKWL | 1860 |
| | MNT | |
| [Y1, s2, Aib16, Q17, E27, K28, | YsQGTFTSDYSKYLD[Aib]QRAQDFVQ | 1861 |
| E29]-GCG-OH | WLEKE | |
| [s2, Aib16, r17, L27, K28]- | HsQGTFTSDYSKYLD[Aib]rRAQDFVQ | 1862 |
| GCG-OH | WLLKT | |
| [s2, Aib16, Aad27, K28]-GCG- | HsQGT FTSDY SKYLD [Aib]RRAQ | 1879 |
| OH | DFVQW L[Aad]KT | |
| [s2, Aib16, G24, L27, K28]- | HsQGT FTSDY SKYLD [Aib]RRAQ | 1880 |
| GCG-OH | DFVGW LLKT | |
| [s2, Aib16, K17, K24, L27, D28] | HsQGT FTSDY SKYLD [Aib]KRAQ | 1881 |
| -GCG-OH | DFVKW LLDT | |

Provided herein is a polypeptide that agonizes a glucagon receptor (“GCGR”) comprising an amino acid sequence disclosed in Table 2A or Table 2B. In some embodiments, the polypeptide comprises an amino acid sequence disclosed in Table 2A. In some embodiments, the polypeptide comprises an amino acid sequence disclosed in Table 2B.

Provided herein is a polypeptide that agonizes a glucagon receptor (“GCGR”) that consists of an amino acid sequence disclosed in Table 2A or Table 2B. In some embodiments, the polypeptide consists of an amino acid sequence disclosed in Table 2A. In some embodiments, the polypeptide consists of an amino acid sequence disclosed in Table 2B.

Provided herein is a polypeptide comprising an amino acid sequence with between three and nine modifications relative to SEQ ID NO: 1576, wherein the modifications are selected from:
tyrosine and phenylalanine at position 1;
d-serine, 2-aminoisobutyric acid, and d-threonine at position 2;
glutamic acid at position 3;
histidine at position 7;
tryptophan at position 10;
glutamic acid at position 15;
2-aminoisobutyric acid, glutamine, homophenylalanine, and glutamic acid at position 16;
lysine, citrulline, glutamine, and alanine at position 17;
2-naphthylalanine, L-4,4′-biphenylalanine, alanine, citrulline, and lysine at position 18;
4-chloro-L-phenylalanine, alanine, d-glutamine, homoserine, histidine, arginine, and glutamic acid at position 20;
glutamic acid, citrulline, and d-aspartic acid at position 21;
tryptophan and β-cyclohexyl-L-alanine at position 22;
aspartic acid, lysine, alanine, 2-aminoisobutyric acid, glycine, histidine, asparagine, threonine, d-glutamine, glutamic acid, arginine, phenylalanine, leucine, serine, tyrosine, valine, isoleucine, homoserine, and 2,3-diaminopropionic acid at position 24;
5-bromo-L-tryptophan, tyrosine, L-beta-homotryptophan, 5-methoxy-L-tryptophan, 5-methyl-L-tryptophan, 6-bromo-L-tryptophan, 6-chloro-L-tryptophan, 6-methyl-L-tryptophan, and 7-bromo-L-tryptophan at position 25;
leucine, glutamic acid, and L-α-aminoadipic acid at position 27;
lysine, aspartic acid, serine, 6-azido-L-lysine, glutamic acid, and alanine at position 28;
glutamic acid, serine, aspartic acid, and alanine at position 29;
an additional amino acid at position 30, wherein the additional amino acid is lysine; and
an additional amino acid at position 31, wherein the additional amino acid is lysine.

In some embodiments, the amino acid sequence comprises between three and eight modifications relative to SEQ ID NO: 1576. In some embodiments, the amino acid sequence comprises between three and seven modifications relative to SEQ ID NO: 1576. In some embodiments, the amino acid sequence comprises between three and six modifications relative to SEQ ID NO: 1576. In some embodiments, the amino acid sequence comprises between three and five modifications relative to SEQ ID NO: 1576. In some embodiments, the amino acid sequence comprises three or four modifications relative to SEQ ID NO: 1576.

In some embodiments, the amino acid sequence comprises three modifications relative to SEQ ID NO: 1576.

In some embodiments, the modifications comprise a d-serine at position 2, a 2-aminoisobutyric acid at position 16, and between one and seven other modifications selected from:
tyrosine and phenylalanine at position 1;
glutamic acid at position 3;
histidine at position 7;
tryptophan at position 10;
glutamic acid at position 15;
lysine, citrulline, glutamine, and alanine at position 17;
2-naphthylalanine, L-4,4′-biphenylalanine, alanine, citrulline, and lysine at position 18;
4-chloro-L-phenylalanine, alanine, d-glutamine, homoserine, histidine, arginine, and glutamic acid at position 20;
glutamic acid, citrulline, and d-aspartic acid at position 21;
tryptophan and β-cyclohexyl-L-alanine at position 22;
aspartic acid, lysine, alanine, 2-aminoisobutyric acid, glycine, histidine, asparagine, threonine, d-glutamine, glutamic acid, arginine, phenylalanine, leucine, serine, tyrosine, valine, isoleucine, homoserine, and 2,3-diaminopropionic acid at position 24;
5-bromo-L-tryptophan, tyrosine, L-beta-homotryptophan, 5-methoxy-L-tryptophan, 5-methyl-L-tryptophan, 6-bromo-L-tryptophan, 6-chloro-L-tryptophan, 6-methyl-L-tryptophan, and 7-bromo-L-tryptophan at position 25;
leucine, glutamic acid, and L-α-aminoadipic acid at position 27;
lysine, aspartic acid, serine, 6-azido-L-lysine, glutamic acid, and alanine at position 28;
glutamic acid, serine, aspartic acid, and alanine at position 29;
an additional amino acid at position 30, wherein the additional amino acid is lysine; and
an additional amino acid at position 31, wherein the additional amino acid is lysine.

In some embodiments, the modifications comprise a d-serine at position 2, a 2-aminoisobutyric acid at position 16, and between one and seven other modifications selected from:
lysine at position 17;
glutamic acid at position 21;
lysine, alanine, and glutamic acid at position 24;
5-bromo-L-tryptophan at position 25;
leucine and glutamic acid at position 27;
lysine, aspartic acid, and glutamic acid at position 28; and
glutamic acid and serine at position 29.

In some embodiments, the modifications comprise a d-serine at position 2, a 2-aminoisobutyric acid at position 16, and between one and six other modifications selected from:
lysine at position 17;
glutamic acid at position 21;
lysine, alanine, and glutamic acid at position 24;
leucine and glutamic acid at position 27;
lysine, aspartic acid, and glutamic acid at position 28; and
glutamic acid and serine at position 29.

In some embodiments, the modifications comprise a d-serine at position 2, a 2-aminoisobutyric acid at position 16, and one or two other modifications selected from:
lysine at position 24; and
lysine and glutamic acid at position 28.

In some embodiments, the modifications comprise d-serine at position 2, 2-aminoisobutyric acid at position 16, and lysine at position 24. In some embodiments, the modifications comprise d-serine at position 2, 2-aminoisobutyric acid at position 16, lysine at position 24, and glutamic acid at position 28.

In some embodiments, the modifications comprise d-serine at position 2, 2-aminoisobutyric acid at position 16, and lysine at position 28.

In some embodiments, the modifications comprise tyrosine at position 1, d-serine at position 2, and 2-aminoisobutyric acid at position 16. In some embodiments, the modifications comprise phenylalanine at position 1, d-serine at position 2, and 2-aminoisobutyric acid at position 16.

In some embodiments, the modifications comprise d-serine at position 2, glutamic acid at position 3, and 2-aminoisobutyric acid at position 16.

In some embodiments, the modifications comprise d-serine at position 2, histidine at position 7, and 2-aminoisobutyric acid at position 16.

In some embodiments, the modifications comprise d-serine at position 2, tryptophan at position 10, and 2-aminoisobutyric acid at position 16.

In some embodiments, the modifications comprise d-serine at position 2, glutamic acid at position 15, and 2-aminoisobutyric acid at position 16.

In some embodiments, the modifications comprise d-serine at position 2, 2-aminoisobutyric acid at position 16, and lysine at position 17. In some embodiments, the modifications comprise d-serine at position 2, 2-aminoisobutyric acid at position 16, and citrulline at position 17. In some embodiments, the modifications comprise d-serine at position 2, 2-aminoisobutyric acid at position 16, and glutamine at position 17. In some embodiments, the modifications comprise d-serine at position 2, 2-aminoisobutyric acid at position 16, and alanine at position 17.

In some embodiments, the modifications comprise d-serine at position 2, 2-aminoisobutyric acid at position 16, and 2-naphthylalanine at position 18. In some embodiments, the modifications comprise d-serine at position 2, 2-aminoisobutyric acid at position 16, and L-4,4′-biphenylalanine at position 18. In some embodiments, the modifications comprise d-serine at position 2, 2-aminoisobutyric acid at position 16, and alanine at position 18. In some embodiments, the modifications comprise d-serine at position 2, 2-aminoisobutyric acid at position 16, and citrulline at position 18. In some embodiments, the modifications comprise d-serine at position 2, 2-aminoisobutyric acid at position 16, and lysine at position 18.

In some embodiments, the modifications comprise d-serine at position 2, 2-aminoisobutyric acid at position 16, and 4-chloro-L-phenylalanine at position 20. In some embodiments, the modifications comprise d-serine at position 2, 2-aminoisobutyric acid at position 16, and alanine at position 20. In some embodiments, the modifications comprise d-serine at position 2, 2-aminoisobutyric acid at position 16, and d-glutamine at position 20. In some embodiments, the modifications comprise d-serine at position 2, 2-aminoisobutyric acid at position 16, and homoserine at position 20. In some embodiments, the modifications comprise d-serine at position 2, 2-aminoisobutyric acid at position 16, and histidine at position 20. In some embodiments, the modifications comprise d-serine at position 2, 2-aminoisobutyric acid at position 16, and arginine at position 20. In some embodiments, the modifications comprise d-serine at position 2, 2-aminoisobutyric acid at position 16, and glutamic acid at position 20.

In some embodiments, the modifications comprise d-serine at position 2, 2-aminoisobutyric acid at position 16, and glutamic acid at position 21. In some embodiments, the modifications comprise d-serine at position 2, 2-aminoisobutyric acid at position 16, and citrulline at position 21. In some embodiments, the modifications comprise d-serine at position 2, 2-aminoisobutyric acid at position 16, and d-aspartic acid at position 21.

In some embodiments, the modifications comprise d-serine at position 2, 2-aminoisobutyric acid at position 16, and tryptophan at position 22. In some embodiments, the modifications comprise d-serine at position 2, 2-aminoisobutyric acid at position 16, and β-cyclohexyl-L-alanine at position 22.

In some embodiments, the modifications comprise d-serine at position 2, 2-aminoisobutyric acid at position 16, and aspartic acid at position 24. In some embodiments, the modifications comprise d-serine at position 2, 2-aminoisobutyric acid at position 16, and lysine at position 24. In some embodiments, the modifications comprise d-serine at position 2, 2-aminoisobutyric acid at position 16, and alanine at position 24. In some embodiments, the modifications comprise d-serine at position 2, 2-aminoisobutyric acid at position 16, and 2-aminoisobutyric acid at position 24. In some embodiments, the modifications comprise d-serine at position 2, 2-aminoisobutyric acid at position 16, and glycine at position 24. In some embodiments, the modifications comprise d-serine at position 2, 2-aminoisobutyric acid at position 16, and histidine at position 24. In some embodiments, the modifications comprise d-serine at position 2, 2-aminoisobutyric acid at position 16, and asparagine at position 24. In some embodiments, the modifications comprise d-serine at position 2, 2-aminoisobutyric acid at position 16, and threonine at position 24. In some embodiments, the modifications comprise d-serine at position 2, 2-aminoisobutyric acid at position 16, and d-glutamine at position 24. In some embodiments, the modifications comprise d-serine at position 2, 2-aminoisobutyric acid at position 16, and glutamic acid at position 24. In some embodiments, the modifications comprise d-serine at position 2, 2-aminoisobutyric acid at position 16, and arginine at position 24. In some embodiments, the modifications comprise d-serine at position 2, 2-aminoisobutyric acid at position 16, and phenylalanine at position 24. In some embodiments, the modifications comprise d-serine at position 2, 2-aminoisobutyric acid at position 16, and leucine at position 24. In some embodiments, the modifications comprise d-serine at position 2, 2-aminoisobutyric acid at position 16, and serine at position 24. In some embodiments, the modifications comprise d-serine at position 2, 2-aminoisobutyric acid at position 16, and tyrosine at position 24. In some embodiments, the modifications comprise d-serine at position 2, 2-aminoisobutyric acid at position 16, and valine at position 24. In some embodiments, the modifications comprise d-serine at position 2, 2-aminoisobutyric acid at position 16, and isoleucine at position 24. In some embodiments, the modifications comprise d-serine at position 2, 2-aminoisobutyric acid at position 16, and homoserine at position 24. In some embodiments, the modifications comprise d-serine at position 2, 2-aminoisobutyric acid at position 16, and 2,3-diaminopropionic acid at position 24.

In some embodiments, the modifications comprise d-serine at position 2, 2-aminoisobutyric acid at position 16, and 5-bromo-L-tryptophan at position 25. In some embodiments, the modifications comprise d-serine at position 2, 2-aminoisobutyric acid at position 16, and tyrosine at position 25. In some embodiments, the modifications comprise d-serine at position 2, 2-aminoisobutyric acid at position 16, and L-beta-homotryptophan at position 25. In some embodiments, the modifications comprise d-serine at position 2, 2-aminoisobutyric acid at position 16, and 5-methoxy-L-tryptophan at position 25. In some embodiments, the modifications comprise d-serine at position 2, 2-aminoisobutyric acid at position 16, and 5-methyl-L-tryptophan at position 25. In some embodiments, the modifications comprise d-serine at position 2, 2-aminoisobutyric acid at position 16, and 6-bromo-L-tryptophan at position 25. In some embodiments, the modifications comprise d-serine at position 2, 2-aminoisobutyric acid at position 16, and 6-chloro-L-tryptophan at position 25. In some embodiments, the modifications comprise d-serine at position 2, 2-aminoisobutyric acid at position 16, and 6-methyl-L-tryptophan at position 25. In some embodiments, the modifications comprise d-serine at position 2, 2-aminoisobutyric acid at position 16, and 7-bromo-L-tryptophan at position 25.

In some embodiments, the modifications comprise d-serine at position 2, 2-aminoisobutyric acid at position 16, and alanine at position 24.

In some embodiments, the modifications comprise d-serine at position 2, 2-aminoisobutyric acid at position 16, and leucine at position 27. In some embodiments, the modifications comprise d-serine at position 2, 2-aminoisobutyric acid at position 16, and glutamic acid at position 27. In some embodiments, the modifications comprise d-serine at position 2, 2-aminoisobutyric acid at position 16, and L-α-aminoadipic acid at position 27.

In some embodiments, the modifications comprise d-serine at position 2, 2-aminoisobutyric acid at position 16, and lysine at position 28. In some embodiments, the modifications comprise d-serine at position 2, 2-aminoisobutyric acid at position 16, and aspartic acid at position 28. In some embodiments, the modifications comprise d-serine at position 2, 2-aminoisobutyric acid at position 16, and serine at position 28. In some embodiments, the modifications comprise d-serine at position 2, 2-aminoisobutyric acid at position 16, and 6-azido-L-lysine at position 28. In some embodiments, the modifications comprise d-serine at position 2, 2-aminoisobutyric acid at position 16, and glutamic acid at position 28. In some embodiments, the modifications comprise d-serine at position 2, 2-aminoisobutyric acid at position 16, and alanine at position 28.

In some embodiments, the modifications comprise d-serine at position 2, 2-aminoisobutyric acid at position 16, and glutamic acid at position 29. In some embodiments, the modifications comprise d-serine at position 2, 2-aminoisobutyric acid at position 16, and serine at position 29. In some embodiments, the modifications comprise d-serine at position 2, 2-aminoisobutyric acid at position 16, and aspartic acid at position 29. In some embodiments, the modifications comprise d-serine at position 2, 2-aminoisobutyric acid at position 16, and alanine at position 29.

In some embodiments, the modifications comprise d-serine at position 2, 2-aminoisobutyric acid at position 16, and an additional amino acid at position 30, wherein the additional amino acid is lysine.

In some embodiments, the modifications comprise d-serine at position 2, 2-aminoisobutyric acid at position 16, and an additional amino acid at position 31, wherein the additional amino acid is lysine. In some embodiments, the modifications comprise d-serine at position 2, 2-aminoisobutyric acid at position 16, an additional amino acid at position 30, and an additional amino acid at position 31, wherein the additional amino acid at position 30 and the additional amino acid at position 31 are both lysine.

In some embodiments, the polypeptide is a linear polypeptide. In some embodiments, the polypeptide is a peptide. In some embodiments, the peptide is a linear peptide.

Provided herein is a polypeptide that agonizes a glucagon receptor (“GCGR”), wherein:
the polypeptide comprises at least 25 amino acids, wherein the polypeptide comprises 5-bromo-tryptophan at position 25; and
the polypeptide has at least 79% sequence identity to the amino acid sequence of SEQ ID NO: 1587.

In some embodiments, the polypeptide is a linear polypeptide. In some embodiments, the polypeptide is a peptide. In some embodiments, the peptide is a linear peptide.

In some embodiments, the polypeptide comprises at least 27 (e.g., at least 27, at least 28, at least 29; 27, 28, 29) amino acids. In some embodiments, the polypeptide comprises at least 28 amino acids. In some embodiments, the polypeptide comprises at least 29 amino acids.

In some embodiments, the polypeptide comprises 29 amino acids.

In some embodiments, the polypeptide has at least 82% sequence identity to the amino acid sequence of SEQ ID NO: 1587. In some embodiments, the polypeptide has at least 86% sequence identity to the amino acid sequence of SEQ ID NO: 1587. In some embodiments, the polypeptide has at least 89% sequence identity to the amino acid sequence of SEQ ID NO: 1587. In some embodiments, the polypeptide has at least 93% sequence identity to the amino acid sequence of SEQ ID NO: 1587. In some embodiments, the polypeptide has at least 96% sequence identity to the amino acid sequence of SEQ ID NO: 1587.

In some embodiments, the polypeptide comprises an amino acid sequence having at most six (e.g., zero, one, two, three, four, five or six) amino acid modifications relative to SEQ ID NO: 1587. In some embodiments, the polypeptide comprises an amino acid sequence having at most five amino acid modifications relative to SEQ ID NO: 1587. In some embodiments, the polypeptide comprises an amino acid sequence having at most four amino acid modifications relative to SEQ ID NO: 1587. In some embodiments, the polypeptide comprises an amino acid sequence having at most three amino acid modifications relative to SEQ ID NO: 1587. In some embodiments, the polypeptide comprises an amino acid sequence having at most two amino acid modifications relative to SEQ ID NO: 1587. In some embodiments, the polypeptide comprises an amino acid sequence having at most one amino acid modification relative to SEQ ID NO: 1587.

In some embodiments, each amino acid modification, if any, is an amino acid substitution. In some embodiments, each amino acid modification, if any, is a conservative amino acid substitution. In some embodiments, each amino acid modification, if any, is a conservative amino acid substitution listed in Table 1.

In some embodiments, the at most one amino acid modification is an amino acid deletion. In some embodiments, the at most one amino acid modification is an amino acid addition.

In some embodiments, the polypeptide comprises one or more (e.g., two or more, three or more, four or more, five or more; one, two, three, four, five, or six) of: d-serine at position 2; 2-aminoisobutyric acid at position 16; lysine at position 17; β-(2-naphthyl)-L-alanine at position 18; aspartic acid, lysine, alanine, or glutamic acid at position 24; and leucine at position 27. In some embodiments, the polypeptide comprises d-serine at position 2. In some embodiments, the polypeptide comprises 2-aminoisobutyric acid at position 16. In some embodiments, the polypeptide comprises lysine at position 17. In some embodiments, the polypeptide comprises β-(2-naphthyl)-L-alanine at position 18. In some embodiments, the polypeptide comprises aspartic acid, lysine, alanine, or glutamic acid at position 24. In some embodiments, the polypeptide comprises aspartic acid at position 24. In some embodiments, the polypeptide comprises lysine at position 24. In some embodiments, the polypeptide comprises alanine at position 24. In some embodiments, the polypeptide comprises glutamic acid at position 24. In some embodiments, the polypeptide comprises leucine at position 27.

In some embodiments, the polypeptide comprises lysine or aspartic acid at position 28. In some embodiments, the polypeptide comprises lysine at position 28. In some embodiments, the polypeptide comprises aspartic acid at position 28.

In some embodiments, the polypeptide comprises d-serine at position 2 and 2-aminoisobutyric acid at position 16. In some embodiments, the polypeptide comprises d-serine at position 2, 2-aminoisobutyric acid at position 16, and leucine at position 27. In some embodiments, the polypeptide further comprises lysine or aspartic acid at position 28. In some embodiments, the polypeptide further comprises lysine at position 28. In some embodiments, the polypeptide comprises further aspartic acid at position 28.

In some embodiments, the polypeptide comprises d-serine at position 2, 2-aminoisobutyric acid at position 16, leucine at position 27, and lysine or aspartic acid at position 28. In some embodiments, the polypeptide comprises d-serine at position 2, 2-aminoisobutyric acid at position 16, leucine at position 27, and lysine at position 28. In some embodiments, the polypeptide comprises d-serine at position 2, 2-aminoisobutyric acid at position 16, leucine at position 27, and aspartic acid at position 28.

In some embodiments, the polypeptide comprises d-serine at position 2, 2-aminoisobutyric acid at position 16, leucine at position 27, and lysine at position 28. In some embodiments, the polypeptide comprises d-serine at position 2, 2-aminoisobutyric acid at position 16, aspartic acid at position 24, leucine at position 27, and lysine at position 28.

In some embodiments, the polypeptide comprises d-serine at position 2, 2-aminoisobutyric acid at position 16, lysine at position 24, leucine at position 27, and aspartic acid at position 28.

In some embodiments, the polypeptide comprises d-serine at position 2, 2-aminoisobutyric acid at position 16, aspartic acid at position 24, leucine at position 27, and lysine at position 28.

In some embodiments, the polypeptide comprises d-serine at position 2, 2-aminoisobutyric acid at position 16, alanine at position 24, and lysine at position 28. In some embodiments, the polypeptide comprises d-serine at position 2, 2-aminoisobutyric acid at position 16, lysine at position 17, alanine at position 24, leucine at position 27, and lysine at position 28. In some embodiments, the polypeptide comprises d-serine at position 2, 2-aminoisobutyric acid at position 16, lysine at position 17, glutamic acid at position 24, leucine at position 27, and lysine at position 28.

In some embodiments, the polypeptide comprises d-serine at position 2, 2-aminoisobutyric acid at position 16, β-(2-naphthyl)-L-alanine at position 18, lysine or alanine at position 24, leucine at position 27, and lysine or aspartic acid at position 28. In some embodiments, the polypeptide comprises d-serine at position 2, 2-aminoisobutyric acid at position 16, β-(2-naphthyl)-L-alanine at position 18, lysine at position 24, leucine at position 27, and aspartic acid at position 28. In some embodiments, the polypeptide comprises d-serine at position 2, 2-aminoisobutyric acid at position 16, β-(2-naphthyl)-L-alanine at position 18, alanine at position 24, leucine at position 27, and lysine at position 28.

In some embodiments, the polypeptide comprises the amino acid sequence of any one of SEQ ID NOs: 1587-1595. In some embodiments, the polypeptide comprises the amino acid sequence of SEQ ID NO: 1587. In some embodiments, the polypeptide comprises the amino acid sequence of SEQ ID NO: 1588. In some embodiments, the polypeptide comprises the amino acid sequence of SEQ ID NO: 1589. In some embodiments, the polypeptide comprises the amino acid sequence of SEQ ID NO: 1590. In some embodiments, the polypeptide comprises the amino acid sequence of SEQ ID NO: 1591. In some embodiments, the polypeptide comprises the amino acid sequence of SEQ ID NO: 1592. In some embodiments, the polypeptide comprises the amino acid sequence of SEQ ID NO: 1593. In some embodiments, the polypeptide comprises the amino acid sequence of SEQ ID NO: 1594. In some embodiments, the polypeptide comprises the amino acid sequence of SEQ ID NO: 1595.

In some embodiments, the polypeptide consists of the amino acid sequence of any one of SEQ ID NOs: 1587-1595. In some embodiments, the polypeptide consists of the amino acid sequence of SEQ ID NO: 1587. In some embodiments, the polypeptide consists of the amino acid sequence of SEQ ID NO: 1588. In some embodiments, the polypeptide consists of the amino acid sequence of SEQ ID NO: 1589. In some embodiments, the polypeptide consists of the amino acid sequence of SEQ ID NO: 1590. In some embodiments, the polypeptide consists of the amino acid sequence of SEQ ID NO: 1591. In some embodiments, the polypeptide consists of the amino acid sequence of SEQ ID NO: 1592. In some embodiments, the polypeptide consists of the amino acid sequence of SEQ ID NO: 1593. In some embodiments, the polypeptide consists of the amino acid sequence of SEQ ID NO: 1594. In some embodiments, the polypeptide consists of the amino acid sequence of SEQ ID NO: 1595.

In some embodiments, the polypeptide agonizes human GCGR (“hGCGR”). In some embodiments, the hGCGR comprises the amino acid sequence of SEQ ID NO: 1576.

In some embodiments, the polypeptide has a hGCGR EC₅₀ of less than or equal to 1 nM.

In some embodiments, the polypeptide has a hGCGR EC₅₀ of less than or equal to 500 pM (e.g., less than or equal to 475 pM, less than or equal to 450 pM, less than or equal to 425 pM, less than or equal to 400 pM, less than or equal to 375 pM, less than or equal to 350 pM, less than or equal to 325 pM, less than or equal to 300 pM, less than or equal to 275 pM, less than or equal to 250 pM, less than or equal to 225 pM, less than or equal to 200 pM, less than or equal to 175 pM, less than or equal to 150 pM, less than or equal to 125 pM, less than or equal to 100 pM). In some embodiments, the hGCGR EC₅₀ is measured in a functional assay described in “EXAMPLE 1: GCG Receptor Agonist Activity.”

In some embodiments, the polypeptide has a hGCGR EC₅₀ of 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 155 pM, 200 pM, 205 pM, 210 pM, 215 pM, 220 pM, 225 pM, 230 pM, 235 pM, 240 pM, 245 pM, 250 pM, 255 pM, 260 pM, 265 pM, 270 pM, 275 pM, 280 pM, 285 pM, 290 pM, 295 pM, 300 pM, 305 pM, 310 pM, 315 pM, 320 pM, 325 pM, 330 pM, 335 pM, 340 pM, 345 pM, 350 pM, 355 pM, 360 pM, 365 pM, 370 pM, 375 pM, 380 pM, 385 pM, 390 pM, 395 pM, 400 pM, 405 pM, 410 pM, 415 pM, 420 pM, 425 pM, 430 pM, 435 pM, 440 pM, 445 pM, 450 pM, 455 pM, 460 pM, 465 pM, 470 pM, 475 pM, 480 pM, 485 pM, 490 pM, 495 pM, or 500 pM. In some embodiments, the hGCGR EC₅₀ is measured in a functional assay described in “EXAMPLE 1: GCG Receptor Agonist Activity.”

In some embodiments, the polypeptide has a human glucagon-like peptide-1 receptor (“hGLP-1R”) EC₅₀:hGCGR EC₅₀ ratio of at least 30:1. In some embodiments, the polypeptide has a human glucagon-like peptide-1 receptor (“hGLP-1R”) EC₅₀:hGCGR EC₅₀ ratio of at least 40:1. In some embodiments, the polypeptide has a hGLP-1R EC₅₀:hGCGR EC₅₀ ratio of at least 50:1. In some embodiments, the polypeptide has a hGLP-1R EC₅₀:hGCGR EC₅₀ ratio of at least 60:1. In some embodiments, the polypeptide has a hGLP-1R EC₅₀:hGCGR EC₅₀ ratio of at least 70:1. In some embodiments, the polypeptide has a hGLP-1R EC₅₀:hGCGR EC₅₀ ratio of at least 80:1. In some embodiments, the polypeptide has a hGLP-1R EC₅₀:hGCGR EC₅₀ ratio of at least 90:1. In some embodiments, the polypeptide has a hGLP-1R EC₅₀:hGCGR EC₅₀ ratio of at least 100:1 In some embodiments, the polypeptide has a hGLP-1R EC₅₀:hGCGR EC₅₀ ratio of at least 150:1. In some embodiments, the polypeptide has a hGLP-1R EC₅₀:hGCGR EC₅₀ ratio of at least 200:1. In some embodiments, the polypeptide has a hGLP-1R EC₅₀:hGCGR EC₅₀ ratio of at least 250:1. In some embodiments, the polypeptide has a hGLP-1R EC₅₀:hGCGR EC₅₀ ratio of at least 300:1. In some embodiments, the polypeptide has a hGLP-1R EC₅₀:hGCGR EC₅₀ ratio of at least 350:1. In some embodiments, the polypeptide has a hGLP-1R EC₅₀:hGCGR EC₅₀ ratio of at least 400:1. In some embodiments, the polypeptide has a hGLP-1R EC₅₀:hGCGR EC₅₀ ratio of at least 450:1. In some embodiments, the polypeptide has a hGLP-1R EC₅₀:hGCGR EC₅₀ ratio of at least 500:1. In some embodiments, the polypeptide has a hGLP-1R EC₅₀:hGCGR EC₅₀ ratio of at least 550:1. In some embodiments, the polypeptide has a hGLP-1R EC₅₀:hGCGR EC₅₀ ratio of at least 650:1. In some embodiments, the polypeptide has a hGLP-1R EC₅₀:hGCGR EC₅₀ ratio of at least 700:1. In some embodiments, the polypeptide has a hGLP-1R EC₅₀:hGCGR EC₅₀ ratio of at least 750:1. In some embodiments, the polypeptide has a hGLP-1R EC₅₀:hGCGR EC₅₀ ratio of at least 800:1. In some embodiments, the polypeptide has a hGLP-1R EC₅₀:hGCGR EC₅₀ ratio of at least 850:1. In some embodiments, the polypeptide has a hGLP-1R EC₅₀:hGCGR EC₅₀ ratio of at least 900:1. In some embodiments, the polypeptide has a hGLP-1R EC₅₀:hGCGR EC₅₀ ratio of at least 950:1. In some embodiments, the polypeptide has a hGLP-1R EC₅₀:hGCGR EC₅₀ ratio of at least 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

In some embodiments, the polypeptide has a hGLP-1R EC₅₀:hGCGR EC₅₀ ratio of 30:1, 40:1, 45:1, 50:1, 55:1, 60:1, 65:1, 70:1, 75:1, 80:1, 85:1, 90:1, 95:1, 100:1, 125:1, 150:1, 175:1, 200:1, 225:1, 250:1, 275:1, 300:1, 325:1, 350:1, 375:1, 400:1, 425:1, 450:1, 475:1, 500:1, 525:1, 550:1, 575:1, 600:1, 625:1, 650:1, 675:1, 700:1, 725:1, 750:1, 775:1, 800:1, 825:1, 850:1, 875:1, 900:1, 925:1, 950:1, 975:1, or 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

Provided herein is a polypeptide that agonizes a GCGR, wherein:
the polypeptide comprises at least 28 amino acids, wherein the polypeptide comprises 2-aminoisobutyric acid at position 16 and lysine, serine, or aspartic acid at position 28; and
the polypeptide has at least 89% sequence identity to the amino acid sequence of SEQ ID NO: 1596.

In some embodiments, the polypeptide comprises lysine at position 28.

In some embodiments, the polypeptide comprises serine at position 28.

In some embodiments, the polypeptide comprises aspartic acid at position 28.

In some embodiments, the polypeptide is a linear polypeptide. In some embodiments, the polypeptide is a peptide. In some embodiments, the peptide is a linear peptide.

In some embodiments, the polypeptide comprises 29 amino acids.

In some embodiments, the polypeptide has at least 93% sequence identity to the amino acid sequence of SEQ ID NO: 1596. In some embodiments, the polypeptide has at least 96% sequence identity to the amino acid sequence of SEQ ID NO: 1596.

In some embodiments, the polypeptide comprises an amino acid sequence having at most three (e.g., zero, one, two, three) amino acid modifications relative to SEQ ID NO: 1596. In some embodiments, the polypeptide comprises an amino acid sequence having at most two amino acid modifications relative to SEQ ID NO: 1596. In some embodiments, the polypeptide comprises an amino acid sequence having at most one amino acid modification relative to SEQ ID NO: 1596.

In some embodiments, each amino acid modification, if any, is an amino acid substitution. In some embodiments, each amino acid modification, if any, is a conservative amino acid substitution. In some embodiments, each amino acid modification, if any, is a conservative amino acid substitution listed in Table 1.

In some embodiments, the at most one amino acid modification is an amino acid deletion. In some embodiments, the at most one amino acid modification is an amino acid addition.

In some embodiments, the polypeptide comprises one or more (e.g., two or more, three of more, four or more, five or more; one, two, three, four, or five) of the following: tyrosine at position 1; d-serine, d-threonine, or 2-aminoisobutyric acid at position 2; histidine at position 7; lysine, citrulline, or glutamine at position 17; β-(2-naphthyl)-L-alanine or β-(4,4′-biphenyl)alanine at position 18; lysine, alanine, asparagine, glutamic acid, glycine, aspartic acid, histidine, threonine, or 2-aminoisobutyric acid at position 24; tyrosine, 5-bromo-tryptophan, or L-beta-homotryptophan at position 25; leucine, glutamic acid, or α-aminoadipic acid at position 27; and alanine, aspartic acid, glutamic acid, or serine at position 29.

In some embodiments, the polypeptide comprises tyrosine at position 1.

In some embodiments, the polypeptide comprises d-serine, d-threonine, or 2-aminoisobutyric acid at position 2. In some embodiments, the polypeptide comprises d-serine at position 2. In some embodiments, the polypeptide comprises d-threonine at position 2. In some embodiments, the polypeptide comprises 2-aminoisobutyric acid at position 2.

In some embodiments, the polypeptide comprises histidine at position 7.

In some embodiments, the polypeptide comprises lysine, citrulline, or glutamine at position 17. In some embodiments, the polypeptide comprises lysine at position 17. In some embodiments, the polypeptide comprises citrulline at position 17. In some embodiments, the polypeptide comprises glutamine at position 17.

In some embodiments, the polypeptide comprises β-(2-naphthyl)-L-alanine or β-(4,4′-biphenyl)alanine at position 18. In some embodiments, the polypeptide comprises β-(2-naphthyl)-L-alanine at position 18. In some embodiments, the polypeptide comprises β-(4,4′-biphenyl)alanine at position 18.

In some embodiments, the polypeptide comprises lysine, alanine, asparagine, glutamic acid, glycine, aspartic acid, histidine, threonine, or 2-aminoisobutyric acid at position 24. In some embodiments, the polypeptide comprises lysine at position 24. In some embodiments, the polypeptide comprises alanine at position 24. In some embodiments, the polypeptide comprises asparagine at position 24. In some embodiments, the polypeptide comprises glutamic acid at position 24. In some embodiments, the polypeptide comprises glycine at position 24. In some embodiments, the polypeptide comprises aspartic acid at position 24. In some embodiments, the polypeptide comprises histidine at position 24. In some embodiments, the polypeptide comprises threonine at position 24. In some embodiments, the polypeptide comprises 2-aminoisobutyric acid at position 24.

In some embodiments, the polypeptide comprises tyrosine, 5-bromo-tryptophan, or L-beta-homotryptophan at position 25. In some embodiments, the polypeptide comprises tyrosine at position 25. In some embodiments, the polypeptide comprises 5-bromo-tryptophan at position 25. In some embodiments, the polypeptide comprises L-beta-homotryptophan at position 25.

In some embodiments, the polypeptide comprises leucine, glutamic acid, or α-aminoadipic acid at position 27. In some embodiments, the polypeptide comprises leucine at position 27. In some embodiments, the polypeptide comprises glutamic acid at position 27. In some embodiments, the polypeptide comprises α-aminoadipic acid at position 27.

In some embodiments, the polypeptide comprises alanine, aspartic acid, glutamic acid, or serine at position 29. In some embodiments, the polypeptide comprises alanine at position 29. In some embodiments, the polypeptide comprises aspartic acid at position 29. In some embodiments, the polypeptide comprises glutamic acid at position 29. In some embodiments, the polypeptide comprises serine at position 29.

In some embodiments, the polypeptide comprises d-serine at position 2, 2-aminoisobutyric acid at position 16, leucine at position 27, and lysine at position 28.

In some embodiments, the polypeptide comprises d-serine at position 2, 2-aminoisobutyric acid at position 16, lysine at position 17, leucine at position 27, and lysine at position 28.

In some embodiments, the polypeptide comprises d-serine at position 2, 2-aminoisobutyric acid at position 16, lysine, alanine, asparagine, glutamic acid, glycine, aspartic acid, histidine, threonine, or 2-aminoisobutyric acid at position 24, and lysine at position 28.

In some embodiments, the polypeptide comprises d-serine at position 2, 2-aminoisobutyric acid at position 16, tyrosine, 5-bromo-tryptophan, or L-beta-homotryptophan at position 25, leucine at position 27, and lysine at position 28.

In some embodiments, the polypeptide comprises d-serine at position 2, 2-aminoisobutyric acid at position 16, lysine at position 28, and alanine, aspartic acid, glutamic acid, or serine at position 29.

In some embodiments, the polypeptide agonizes human GCGR (“hGCGR”). In some embodiments, the hGCGR comprises the amino acid sequence of SEQ ID NO: 1576.

In some embodiments, the polypeptide has a hGCGR EC₅₀ of less than or equal to 1 nM.

In some embodiments, the polypeptide has a hGCGR EC₅₀ of less than or equal to 500 pM (e.g., less than or equal to 475 pM, less than or equal to 450 pM, less than or equal to 425 pM, less than or equal to 400 pM, less than or equal to 375 pM, less than or equal to 350 pM, less than or equal to 325 pM, less than or equal to 300 pM, less than or equal to 275 pM, less than or equal to 250 pM, less than or equal to 225 pM, less than or equal to 200 pM, less than or equal to 175 pM, less than or equal to 150 pM, less than or equal to 125 pM, less than or equal to 100 pM). In some embodiments, the hGCGR EC₅₀ is measured in a functional assay described in “EXAMPLE 1: GCG Receptor Agonist Activity.”

In some embodiments, the polypeptide has a hGCGR EC₅₀ of 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 155 pM, 200 pM, 205 pM, 210 pM, 215 pM, 220 pM, 225 pM, 230 pM, 235 pM, 240 pM, 245 pM, 250 pM, 255 pM, 260 pM, 265 pM, 270 pM, 275 pM, 280 pM, 285 pM, 290 pM, 295 pM, 300 pM, 305 pM, 310 pM, 315 pM, 320 pM, 325 pM, 330 pM, 335 pM, 340 pM, 345 pM, 350 pM, 355 pM, 360 pM, 365 pM, 370 pM, 375 pM, 380 pM, 385 pM, 390 pM, 395 pM, 400 pM, 405 pM, 410 pM, 415 pM, 420 pM, 425 pM, 430 pM, 435 pM, 440 pM, 445 pM, 450 pM, 455 pM, 460 pM, 465 pM, 470 pM, 475 pM, 480 pM, 485 pM, 490 pM, 495 pM, or 500 pM. In some embodiments, the hGCGR EC₅₀ is measured in a functional assay described in “EXAMPLE 1: GCG Receptor Agonist Activity.”

In some embodiments, the polypeptide has a human glucagon-like peptide-1 receptor (“hGLP-1R”) EC₅₀:hGCGR EC₅₀ ratio of at least 30:1. In some embodiments, the polypeptide has a hGLP-1R EC₅₀:hGCGR EC₅₀ ratio of at least 40:1. In some embodiments, the polypeptide has a hGLP-1R EC₅₀:hGCGR EC₅₀ ratio of at least 50:1. In some embodiments, the polypeptide has a hGLP-1R EC₅₀:hGCGR EC₅₀ ratio of at least 60:1. In some embodiments, the polypeptide has a hGLP-1R EC₅₀:hGCGR EC₅₀ ratio of at least 70:1. In some embodiments, the polypeptide has a hGLP-1R EC₅₀:hGCGR EC₅₀ ratio of at least 80:1. In some embodiments, the polypeptide has a hGLP-1R EC₅₀:hGCGR EC₅₀ ratio of at least 90:1. In some embodiments, the polypeptide has a hGLP-1R EC₅₀:hGCGR EC₅₀ ratio of at least 100:1. In some embodiments, the polypeptide has a hGLP-1R EC₅₀:hGCGR EC₅₀ ratio of at least 150:1. In some embodiments, the polypeptide has a hGLP-1R EC₅₀:hGCGR EC₅₀ ratio of at least 200:1. In some embodiments, the polypeptide has a hGLP-1R EC₅₀:hGCGR EC₅₀ ratio of at least 250:1. In some embodiments, the polypeptide has a hGLP-1R EC₅₀:hGCGR EC₅₀ ratio of at least 300:1. In some embodiments, the polypeptide has a hGLP-1R EC₅₀:hGCGR EC₅₀ ratio of at least 350:1. In some embodiments, the polypeptide has a hGLP-1R EC₅₀:hGCGR EC₅₀ ratio of at least 400:1. In some embodiments, the polypeptide has a hGLP-1R EC₅₀:hGCGR EC₅₀ ratio of at least 450:1. In some embodiments, the polypeptide has a hGLP-1R EC₅₀:hGCGR EC₅₀ ratio of at least 500:1. In some embodiments, the polypeptide has a hGLP-1R EC₅₀:hGCGR EC₅₀ ratio of at least 550:1. In some embodiments, the polypeptide has a hGLP-1R EC₅₀:hGCGR EC₅₀ ratio of at least 650:1. In some embodiments, the polypeptide has a hGLP-1R EC₅₀:hGCGR EC₅₀ ratio of at least 700:1. In some embodiments, the polypeptide has a hGLP-1R EC₅₀:hGCGR EC₅₀ ratio of at least 750:1. In some embodiments, the polypeptide has a hGLP-1R EC₅₀:hGCGR EC₅₀ ratio of at least 800:1. In some embodiments, the polypeptide has a hGLP-1R EC₅₀:hGCGR EC₅₀ ratio of at least 850:1. In some embodiments, the polypeptide has a hGLP-1R EC₅₀:hGCGR EC₅₀ ratio of at least 900:1. In some embodiments, the polypeptide has a hGLP-1R EC₅₀:hGCGR EC₅₀ ratio of at least 950:1. In some embodiments, the polypeptide has a hGLP-1R EC₅₀:hGCGR EC₅₀ ratio of at least 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

In some embodiments, the polypeptide has a hGLP-1R EC₅₀:hGCGR EC₅₀ ratio of 30:1, 40:1, 45:1, 50:1, 55:1, 60:1, 65:1, 70:1, 75:1, 80:1, 85:1, 90:1, 95:1, 100:1, 125:1, 150:1, 175:1, 200:1, 225:1, 250:1, 275:1, 300:1, 325:1, 350:1, 375:1, 400:1, 425:1, 450:1, 475:1, 500:1, 525:1, 550:1, 575:1, 600:1, 625:1, 650:1, 675:1, 700:1, 725:1, 750:1, 775:1, 800:1, 825:1, 850:1, 875:1, 900:1, 925:1, 950:1, 975:1, or 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

Provided herein is a polypeptide that agonizes a GCGR, wherein:
the polypeptide comprises at least 28 amino acids, wherein the polypeptide comprises 2-aminoisobutyric acid at position 16, leucine at position 27, and lysine at position 28; and
the polypeptide has at least 89% sequence identity to the amino acid sequence of SEQ ID NO: 1615.

In some embodiments, the polypeptide is a linear polypeptide. In some embodiments, the polypeptide is a peptide. In some embodiments, the peptide is a linear peptide.

In some embodiments, the polypeptide comprises 29 amino acids.

In some embodiments, the polypeptide has at least 93% sequence identity to the amino acid sequence of SEQ ID NO: 1615. In some embodiments, the polypeptide has at least 96% sequence identity to the amino acid sequence of SEQ ID NO: 1615.

In some embodiments, the polypeptide comprises an amino acid sequence having at most three (e.g., zero, one, two, three) amino acid modifications relative to SEQ ID NO: 1615. In some embodiments, the polypeptide comprises an amino acid sequence having at most two amino acid modifications relative to SEQ ID NO: 1615. In some embodiments, the polypeptide comprises an amino acid sequence having at most one amino acid modification relative to SEQ ID NO: 1615.

In some embodiments, each amino acid modification, if any, is an amino acid substitution. In some embodiments, each amino acid modification, if any, is a conservative amino acid substitution. In some embodiments, each amino acid modification, if any, is a conservative amino acid substitution listed in Table 1.

In some embodiments, the at most one amino acid modification is an amino acid deletion. In some embodiments, the at most one amino acid modification is an amino acid addition.

In some embodiments, the polypeptide comprises d-serine at position 2.

In some embodiments, the polypeptide comprises aspartic acid or glutamic acid at position 24. In some embodiments, the polypeptide comprises aspartic acid at position 24. In some embodiments, the polypeptide comprises glutamic acid at position 24.

In some embodiments, the polypeptide comprises d-serine at position 2 and aspartic acid or glutamic acid at position 24. In some embodiments, the polypeptide comprises d-serine at position 2 and aspartic acid at position 24. In some embodiments, the polypeptide comprises d-serine at position 2 and glutamic acid at position 24.

In some embodiments, the polypeptide comprises lysine at position 17.

In some embodiments, the polypeptide further comprises d-serine at position 2, lysine at position 17, and aspartic acid at position 24.

In some embodiments, the polypeptide agonizes human GCGR (“hGCGR”). In some embodiments, the hGCGR comprises the amino acid sequence of SEQ ID NO: 1576.

In some embodiments, the polypeptide has a hGCGR EC₅₀ of less than or equal to 1 nM.

In some embodiments, the polypeptide has a hGCGR EC₅₀ of less than or equal to 500 pM (e.g., less than or equal to 475 pM, less than or equal to 450 pM, less than or equal to 425 pM, less than or equal to 400 pM, less than or equal to 375 pM, less than or equal to 350 pM, less than or equal to 325 pM, less than or equal to 300 pM, less than or equal to 275 pM, less than or equal to 250 pM, less than or equal to 225 pM, less than or equal to 200 pM, less than or equal to 175 pM, less than or equal to 150 pM, less than or equal to 125 pM, less than or equal to 100 pM). In some embodiments, the hGCGR EC₅₀ is measured in a functional assay described in “EXAMPLE 1: GCG Receptor Agonist Activity.”

In some embodiments, the polypeptide has a hGCGR EC₅₀ of 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 155 pM, 200 pM, 205 pM, 210 pM, 215 pM, 220 pM, 225 pM, 230 pM, 235 pM, 240 pM, 245 pM, 250 pM, 255 pM, 260 pM, 265 pM, 270 pM, 275 pM, 280 pM, 285 pM, 290 pM, 295 pM, 300 pM, 305 pM, 310 pM, 315 pM, 320 pM, 325 pM, 330 pM, 335 pM, 340 pM, 345 pM, 350 pM, 355 pM, 360 pM, 365 pM, 370 pM, 375 pM, 380 pM, 385 pM, 390 pM, 395 pM, 400 pM, 405 pM, 410 pM, 415 pM, 420 pM, 425 pM, 430 pM, 435 pM, 440 pM, 445 pM, 450 pM, 455 pM, 460 pM, 465 pM, 470 pM, 475 pM, 480 pM, 485 pM, 490 pM, 495 pM, or 500 pM. In some embodiments, the hGCGR EC₅₀ is measured in a functional assay described in “EXAMPLE 1: GCG Receptor Agonist Activity.”

In some embodiments, the polypeptide has a human glucagon-like peptide-1 receptor (“hGLP-1R”) EC₅₀:hGCGR EC₅₀ ratio of at least 30:1. In some embodiments, the polypeptide has a hGLP-1R EC₅₀:hGCGR EC₅₀ ratio of at least 40:1. In some embodiments, the polypeptide has a hGLP-1R EC₅₀:hGCGR EC₅₀ ratio of at least 50:1. In some embodiments, the polypeptide has a hGLP-1R EC₅₀:hGCGR EC₅₀ ratio of at least 60:1. In some embodiments, the polypeptide has a hGLP-1R EC₅₀:hGCGR EC₅₀ ratio of at least 70:1. In some embodiments, the polypeptide has a hGLP-1R EC₅₀:hGCGR EC₅₀ ratio of at least 80:1. In some embodiments, the polypeptide has a hGLP-1R EC₅₀:hGCGR EC₅₀ ratio of at least 90:1. In some embodiments, the polypeptide has a hGLP-1R EC₅₀:hGCGR EC₅₀ ratio of at least 100:1. In some embodiments, the polypeptide has a hGLP-1R EC₅₀:hGCGR EC₅₀ ratio of at least 150:1. In some embodiments, the polypeptide has a hGLP-1R EC₅₀:hGCGR EC₅₀ ratio of at least 200:1. In some embodiments, the polypeptide has a hGLP-1R EC₅₀:hGCGR EC₅₀ ratio of at least 250:1. In some embodiments, the polypeptide has a hGLP-1R EC₅₀:hGCGR EC₅₀ ratio of at least 300:1. In some embodiments, the polypeptide has a hGLP-1R EC₅₀:hGCGR EC₅₀ ratio of at least 350:1. In some embodiments, the polypeptide has a hGLP-1R EC₅₀:hGCGR EC₅₀ ratio of at least 400:1. In some embodiments, the polypeptide has a hGLP-1R EC₅₀:hGCGR EC₅₀ ratio of at least 450:1. In some embodiments, the polypeptide has a hGLP-1R EC₅₀:hGCGR EC₅₀ ratio of at least 500:1. In some embodiments, the polypeptide has a hGLP-1R EC₅₀:hGCGR EC₅₀ ratio of at least 550:1. In some embodiments, the polypeptide has a hGLP-1R EC₅₀:hGCGR EC₅₀ ratio of at least 650:1. In some embodiments, the polypeptide has a hGLP-1R EC₅₀:hGCGR EC₅₀ ratio of at least 700:1. In some embodiments, the polypeptide has a hGLP-1R EC₅₀:hGCGR EC₅₀ ratio of at least 750:1. In some embodiments, the polypeptide has a hGLP-1R EC₅₀:hGCGR EC₅₀ ratio of at least 800:1. In some embodiments, the polypeptide has a hGLP-1R EC₅₀:hGCGR EC₅₀ ratio of at least 850:1. In some embodiments, the polypeptide has a hGLP-1R EC₅₀:hGCGR EC₅₀ ratio of at least 900:1. In some embodiments, the polypeptide has a hGLP-1R EC₅₀:hGCGR EC₅₀ ratio of at least 950:1. In some embodiments, the polypeptide has a hGLP-1R EC₅₀:hGCGR EC₅₀ ratio of at least 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

In some embodiments, the polypeptide has a hGLP-1R EC₅₀:hGCGR EC₅₀ ratio of 30:1, 40:1, 45:1, 50:1, 55:1, 60:1, 65:1, 70:1, 75:1, 80:1, 85:1, 90:1, 95:1, 100:1, 125:1, 150:1, 175:1, 200:1, 225:1, 250:1, 275:1, 300:1, 325:1, 350:1, 375:1, 400:1, 425:1, 450:1, 475:1, 500:1, 525:1, 550:1, 575:1, 600:1, 625:1, 650:1, 675:1, 700:1, 725:1, 750:1, 775:1, 800:1, 825:1, 850:1, 875:1, 900:1, 925:1, 950:1, 975:1, or 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

Provided herein is a polypeptide that agonizes a GCGR, wherein:
the polypeptide comprises at least 28 amino acids, wherein the polypeptide comprises 2-aminoisobutyric acid at position 16, aspartic acid at position 24, and lysine at position 28; and
the polypeptide has at least 89% sequence identity to the amino acid sequence of SEQ ID NO: 1626.

In some embodiments, the polypeptide is a linear polypeptide. In some embodiments, the polypeptide is a peptide. In some embodiments, the peptide is a linear peptide.

In some embodiments, the polypeptide comprises 29 amino acids.

In some embodiments, the polypeptide has at least 93% sequence identity to the amino acid sequence of SEQ ID NO: 1626. In some embodiments, the polypeptide has at least 96% sequence identity to the amino acid sequence of SEQ ID NO: 1626.

In some embodiments, the polypeptide comprises an amino acid sequence having at most three (e.g., zero, one, two, three) amino acid modifications relative to SEQ ID NO: 1626. In some embodiments, the polypeptide comprises an amino acid sequence having at most two amino acid modifications relative to SEQ ID NO: 1626. In some embodiments, the polypeptide comprises an amino acid sequence having at most one amino acid modification relative to SEQ ID NO: 1626.

In some embodiments, each amino acid modification, if any, is an amino acid substitution. In some embodiments, each amino acid modification, if any, is a conservative amino acid substitution. In some embodiments, each amino acid modification, if any, is a conservative amino acid substitution listed in Table 1.

In some embodiments, the at most one amino acid modification is an amino acid deletion. In some embodiments, the at most one amino acid modification is an amino acid addition.

In some embodiments, the polypeptide comprises d-serine at position 2.

In some embodiments, the polypeptide comprises lysine at position 17.

In some embodiments, the polypeptide further comprises leucine at position 27.

In some embodiments, the polypeptide agonizes human GCGR (“hGCGR”). In some embodiments, the hGCGR comprises the amino acid sequence of SEQ ID NO: 1576.

In some embodiments, the polypeptide has a hGCGR EC₅₀ of less than or equal to 1 nM.

In some embodiments, the polypeptide has a hGCGR EC₅₀ of less than or equal to 500 pM (e.g., less than or equal to 475 pM, less than or equal to 450 pM, less than or equal to 425 pM, less than or equal to 400 pM, less than or equal to 375 pM, less than or equal to 350 pM, less than or equal to 325 pM, less than or equal to 300 pM, less than or equal to 275 pM, less than or equal to 250 pM, less than or equal to 225 pM, less than or equal to 200 pM, less than or equal to 175 pM, less than or equal to 150 pM, less than or equal to 125 pM, less than or equal to 100 pM). In some embodiments, the hGCGR EC₅₀ is measured in a functional assay described in “EXAMPLE 1: GCG Receptor Agonist Activity.”

In some embodiments, the polypeptide has a hGCGR EC₅₀ of 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 155 pM, 200 pM, 205 pM, 210 pM, 215 pM, 220 pM, 225 pM, 230 pM, 235 pM, 240 pM, 245 pM, 250 pM, 255 pM, 260 pM, 265 pM, 270 pM, 275 pM, 280 pM, 285 pM, 290 pM, 295 pM, 300 pM, 305 pM, 310 pM, 315 pM, 320 pM, 325 pM, 330 pM, 335 pM, 340 pM, 345 pM, 350 pM, 355 pM, 360 pM, 365 pM, 370 pM, 375 pM, 380 pM, 385 pM, 390 pM, 395 pM, 400 pM, 405 pM, 410 pM, 415 pM, 420 pM, 425 pM, 430 pM, 435 pM, 440 pM, 445 pM, 450 pM, 455 pM, 460 pM, 465 pM, 470 pM, 475 pM, 480 pM, 485 pM, 490 pM, 495 pM, or 500 pM. In some embodiments, the hGCGR EC₅₀ is measured in a functional assay described in “EXAMPLE 1: GCG Receptor Agonist Activity.”

In some embodiments, the polypeptide has a human glucagon-like peptide-1 receptor (“hGLP-1R”) EC₅₀:hGCGR EC₅₀ ratio of at least 30:1. In some embodiments, the polypeptide has a hGLP-1R EC₅₀:hGCGR EC₅₀ ratio of at least 40:1. In some embodiments, the polypeptide has a hGLP-1R EC₅₀:hGCGR EC₅₀ ratio of at least 50:1. In some embodiments, the polypeptide has a hGLP-1R EC₅₀:hGCGR EC₅₀ ratio of at least 60:1. In some embodiments, the polypeptide has a hGLP-1R EC₅₀:hGCGR EC₅₀ ratio of at least 70:1. In some embodiments, the polypeptide has a hGLP-1R EC₅₀:hGCGR EC₅₀ ratio of at least 80:1. In some embodiments, the polypeptide has a hGLP-1R EC₅₀:hGCGR EC₅₀ ratio of at least 90:1. In some embodiments, the polypeptide has a hGLP-1R EC₅₀:hGCGR EC₅₀ ratio of at least 100:1. In some embodiments, the polypeptide has a hGLP-1R EC₅₀:hGCGR EC₅₀ ratio of at least 150:1. In some embodiments, the polypeptide has a hGLP-1R EC₅₀:hGCGR EC₅₀ ratio of at least 200:1. In some embodiments, the polypeptide has a hGLP-1R EC₅₀:hGCGR EC₅₀ ratio of at least 250:1. In some embodiments, the polypeptide has a hGLP-1R EC₅₀:hGCGR EC₅₀ ratio of at least 300:1. In some embodiments, the polypeptide has a hGLP-1R EC₅₀:hGCGR EC₅₀ ratio of at least 350:1. In some embodiments, the polypeptide has a hGLP-1R EC₅₀:hGCGR EC₅₀ ratio of at least 400:1. In some embodiments, the polypeptide has a hGLP-1R EC₅₀:hGCGR EC₅₀ ratio of at least 450:1. In some embodiments, the polypeptide has a hGLP-1R EC₅₀:hGCGR EC₅₀ ratio of at least 500:1. In some embodiments, the polypeptide has a hGLP-1R EC₅₀:hGCGR EC₅₀ ratio of at least 550:1. In some embodiments, the polypeptide has a hGLP-1R EC₅₀:hGCGR EC₅₀ ratio of at least 650:1. In some embodiments, the polypeptide has a hGLP-1R EC₅₀:hGCGR EC₅₀ ratio of at least 700:1. In some embodiments, the polypeptide has a hGLP-1R EC₅₀:hGCGR EC₅₀ ratio of at least 750:1. In some embodiments, the polypeptide has a hGLP-1R EC₅₀:hGCGR EC₅₀ ratio of at least 800:1. In some embodiments, the polypeptide has a hGLP-1R EC₅₀:hGCGR EC₅₀ ratio of at least 850:1. In some embodiments, the polypeptide has a hGLP-1R EC₅₀:hGCGR EC₅₀ ratio of at least 900:1. In some embodiments, the polypeptide has a hGLP-1R EC₅₀:hGCGR EC₅₀ ratio of at least 950:1. In some embodiments, the polypeptide has a hGLP-1R EC₅₀:hGCGR EC₅₀ ratio of at least 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

In some embodiments, the polypeptide has a hGLP-1R EC₅₀:hGCGR EC₅₀ ratio of 30:1, 40:1, 45:1, 50:1, 55:1, 60:1, 65:1, 70:1, 75:1, 80:1, 85:1, 90:1, 95:1, 100:1, 125:1, 150:1, 175:1, 200:1, 225:1, 250:1, 275:1, 300:1, 325:1, 350:1, 375:1, 400:1, 425:1, 450:1, 475:1, 500:1, 525:1, 550:1, 575:1, 600:1, 625:1, 650:1, 675:1, 700:1, 725:1, 750:1, 775:1, 800:1, 825:1, 850:1, 875:1, 900:1, 925:1, 950:1, 975:1, or 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.

Provided herein is a polypeptide that agonizes a GCGR, wherein:
the polypeptide comprises at least 28 amino acids, wherein the polypeptide comprises a d-serine at position 2 and a 2-aminoisobutyric acid at position 16; and
the polypeptide has at least 89% sequence identity to the amino acid sequence of SEQ ID NO: 1822.

In some embodiments, the polypeptide is a linear polypeptide. In some embodiments, the polypeptide is a peptide. In some embodiments, the peptide is a linear peptide.

In some embodiments, the polypeptide comprises 29 amino acids.

In some embodiments, the polypeptide has at least 93% sequence identity to the amino acid sequence of SEQ ID NO: 1822. In some embodiments, the polypeptide has at least 96% sequence identity to the amino acid sequence of SEQ ID NO: 1822.

In some embodiments, the polypeptide comprises an amino acid sequence having at most three (e.g., zero, one, two, three) amino acid modifications relative to SEQ ID NO: 1822. In some embodiments, the polypeptide comprises an amino acid sequence having at most two amino acid modifications relative to SEQ ID NO: 1822. In some embodiments, the polypeptide comprises an amino acid sequence having at most one amino acid modification relative to SEQ ID NO: 1615.

In some embodiments, each amino acid modification, if any, is an amino acid substitution. In some embodiments, each amino acid modification, if any, is a conservative amino acid substitution. In some embodiments, each amino acid modification, if any, is a conservative amino acid substitution listed in Table 1.

In some embodiments, the at most one amino acid modification is an amino acid deletion. In some embodiments, the at most one amino acid modification is an amino acid addition.

In some embodiments, the polypeptide further comprises lysine at position 17, glutamic acid at position 21, lysine at position 24, leucine at position 27, glutamic acid, alanine, or lysine at position 28, serine or threonine at position 29, or a combination of any of the foregoing.

In some embodiments, the polypeptide further comprises lysine at position 17, glutamic acid at position 21, lysine at position 24, leucine at position 27, glutamic acid at position 28, serine at position 29, or a combination of any of the foregoing.

In some embodiments, the polypeptide further comprises lysine at position 17. In some embodiments, the polypeptide further comprises glutamic acid at position 21. In some embodiments, the polypeptide further comprises lysine at position 24. In some embodiments, the polypeptide further comprises leucine at position 27. In some embodiments, the polypeptide further comprises glutamic acid at position 28. In some embodiments, the polypeptide further comprises serine at position 29.

In some embodiments, the polypeptide further comprises lysine at position 17, glutamic acid at position 21, leucine at position 27, and glutamic acid at position 28.

In some embodiments, the polypeptide further comprises lysine at position 17, glutamic acid at position 21, leucine at position 27, glutamic acid at position 28, and lysine at position 24 or position 28.

In some embodiments, the polypeptide has a hGCGR EC₅₀ of less than or equal to 1 nM.

In some embodiments, the polypeptide has a hGCGR EC₅₀ of less than or equal to 500 pM (e.g., less than or equal to 475 pM, less than or equal to 450 pM, less than or equal to 425 pM, less than or equal to 400 pM, less than or equal to 375 pM, less than or equal to 350 pM, less than or equal to 325 pM, less than or equal to 300 pM, less than or equal to 275 pM, less than or equal to 250 pM, less than or equal to 225 pM, less than or equal to 200 pM, less than or equal to 175 pM, less than or equal to 150 pM, less than or equal to 125 pM, less than or equal to 100 pM). In some embodiments, the hGCGR EC₅₀ is measured in a functional assay described in “EXAMPLE 1: GCG Receptor Agonist Activity.”

In some embodiments, the polypeptide has a hGCGR EC₅₀ of 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 155 pM, 200 pM, 205 pM, 210 pM, 215 pM, 220 pM, 225 pM, 230 pM, 235 pM, 240 pM, 245 pM, 250 pM, 255 pM, 260 pM, 265 pM, 270 pM, 275 pM, 280 pM, 285 pM, 290 pM, 295 pM, 300 pM, 305 pM, 310 pM, 315 pM, 320 pM, 325 pM, 330 pM, 335 pM, 340 pM, 345 pM, 350 pM, 355 pM, 360 pM, 365 pM, 370 pM, 375 pM, 380 pM, 385 pM, 390 pM, 395 pM, 400 pM, 405 pM, 410 pM, 415 pM, 420 pM, 425 pM, 430 pM, 435 pM, 440 pM, 445 pM, 450 pM, 455 pM, 460 pM, 465 pM, 470 pM, 475 pM, 480 pM, 485 pM, 490 pM, 495 pM, or 500 pM. In some embodiments, the hGCGR EC₅₀ is measured in a functional assay described in “EXAMPLE 1: GCG Receptor Agonist Activity.”

In some embodiments, the polypeptide has a human glucagon-like peptide-1 receptor (“hGLP-1R”) EC₅₀:hGCGR EC₅₀ ratio of at least 30:1. In some embodiments, the polypeptide has a hGLP-1R EC₅₀:hGCGR EC₅₀ ratio of at least 40:1. In some embodiments, the polypeptide has a hGLP-1R EC₅₀:hGCGR EC₅₀ ratio of at least 50:1. In some embodiments, the polypeptide has a hGLP-1R EC₅₀:hGCGR EC₅₀ ratio of at least 60:1. In some embodiments, the polypeptide has a hGLP-1R EC₅₀:hGCGR EC₅₀ ratio of at least 70:1. In some embodiments, the polypeptide has a hGLP-1R EC₅₀:hGCGR EC₅₀ ratio of at least 80:1. In some embodiments, the polypeptide has a hGLP-1R EC₅₀:hGCGR EC₅₀ ratio of at least 90:1. In some embodiments, the polypeptide has a hGLP-1R EC₅₀:hGCGR EC₅₀ ratio of at least 100:1. In some embodiments, the polypeptide has a hGLP-1R EC₅₀:hGCGR EC₅₀ ratio of at least 150:1. In some embodiments, the polypeptide has a hGLP-1R EC₅₀:hGCGR EC₅₀ ratio of at least 200:1. In some embodiments, the polypeptide has a hGLP-1R EC₅₀:hGCGR EC₅₀ ratio of at least 250:1. In some embodiments, the polypeptide has a hGLP-1R EC₅₀:hGCGR EC₅₀ ratio of at least 300:1. In some embodiments, the polypeptide has a hGLP-1R EC₅₀:hGCGR EC₅₀ ratio of at least 350:1. In some embodiments, the polypeptide has a hGLP-1R EC₅₀:hGCGR EC₅₀ ratio of at least 400:1. In some embodiments, the polypeptide has a hGLP-1R EC₅₀:hGCGR EC₅₀ ratio of at least 450:1. In some embodiments, the polypeptide has a hGLP-1R EC₅₀:hGCGR EC₅₀ ratio of at least 500:1. In some embodiments, the polypeptide has a hGLP-1R EC₅₀:hGCGR EC₅₀ ratio of at least 550:1. In some embodiments, the polypeptide has a hGLP-1R EC₅₀:hGCGR EC₅₀ ratio of at least 650:1. In some embodiments, the polypeptide has a hGLP-1R EC₅₀:hGCGR EC₅₀ ratio of at least 700:1. In some embodiments, the polypeptide has a hGLP-1R EC₅₀:hGCGR EC₅₀ ratio of at least 750:1. In some embodiments, the polypeptide has a hGLP-1R EC₅₀:hGCGR EC₅₀ ratio of at least 800:1. In some embodiments, the polypeptide has a hGLP-1R EC₅₀:hGCGR EC₅₀ ratio of at least 850:1. In some embodiments, the polypeptide has a hGLP-1R EC₅₀:hGCGR EC₅₀ ratio of at least 900:1. In some embodiments, the polypeptide has a hGLP-1R EC₅₀:hGCGR EC₅₀ ratio of at least 950:1. In some embodiments, the polypeptide has a hGLP-1R EC₅₀:hGCGR EC₅₀ ratio of at least 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

In some embodiments, the polypeptide has a hGLP-1R EC₅₀:hGCGR EC₅₀ ratio of 30:1, 40:1, 45:1, 50:1, 55:1, 60:1, 65:1, 70:1, 75:1, 80:1, 85:1, 90:1, 95:1, 100:1, 125:1, 150:1, 175:1, 200:1, 225:1, 250:1, 275:1, 300:1, 325:1, 350:1, 375:1, 400:1, 425:1, 450:1, 475:1, 500:1, 525:1, 550:1, 575:1, 600:1, 625:1, 650:1, 675:1, 700:1, 725:1, 750:1, 775:1, 800:1, 825:1, 850:1, 875:1, 900:1, 925:1, 950:1, 975:1, or 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

Provided herein is a polypeptide that agonizes a GCGR comprising the amino acid sequence of any one of SEQ ID NOs: 1587-1627, 1747-1840, 1859-1862, or 1879-1881. In some embodiments, the polypeptide comprises the amino acid sequence of any one of SEQ ID NOs: 1587-1627 or 1747. In some embodiments, the polypeptide comprises the amino acid sequence of any one of SEQ ID NOs: 1748-1840, 1859-1862, or 1879-1881.

In some embodiments, the polypeptide comprises the amino acid sequence of SEQ ID NO: 1587. In some embodiments, the polypeptide comprises the amino acid sequence of SEQ ID NO: 1588. In some embodiments, the polypeptide comprises the amino acid sequence of SEQ ID NO: 1589. In some embodiments, the polypeptide comprises the amino acid sequence of SEQ ID NO: 1590. In some embodiments, the polypeptide comprises the amino acid sequence of SEQ ID NO: 1591. In some embodiments, the polypeptide comprises the amino acid sequence of SEQ ID NO: 1592. In some embodiments, the polypeptide comprises the amino acid sequence of SEQ ID NO: 1593. In some embodiments, the polypeptide comprises the amino acid sequence of SEQ ID NO: 1594. In some embodiments, the polypeptide comprises the amino acid sequence of SEQ ID NO: 1595. In some embodiments, the polypeptide comprises the amino acid sequence of SEQ ID NO: 1596. In some embodiments, the polypeptide comprises the amino acid sequence of SEQ ID NO: 1597. In some embodiments, the polypeptide comprises the amino acid sequence of SEQ ID NO: 1598. In some embodiments, the polypeptide comprises the amino acid sequence of SEQ ID NO: 1599. In some embodiments, the polypeptide comprises the amino acid sequence of SEQ ID NO: 1600. In some embodiments, the polypeptide comprises the amino acid sequence of SEQ ID NO: 1601. In some embodiments, the polypeptide comprises the amino acid sequence of SEQ ID NO: 1602. In some embodiments, the polypeptide comprises the amino acid sequence of SEQ ID NO: 1603. In some embodiments, the polypeptide comprises the amino acid sequence of SEQ ID NO: 1604. In some embodiments, the polypeptide comprises the amino acid sequence of SEQ ID NO: 1605. In some embodiments, the polypeptide comprises the amino acid sequence of SEQ ID NO: 1606. In some embodiments, the polypeptide comprises the amino acid sequence of SEQ ID NO: 1607. In some embodiments, the polypeptide comprises the amino acid sequence of SEQ ID NO: 1608. In some embodiments, the polypeptide comprises the amino acid sequence of SEQ ID NO: 1609. In some embodiments, the polypeptide comprises the amino acid sequence of SEQ ID NO: 1610. In some embodiments, the polypeptide comprises the amino acid sequence of SEQ ID NO: 1611. In some embodiments, the polypeptide comprises the amino acid sequence of SEQ ID NO: 1612. In some embodiments, the polypeptide comprises the amino acid sequence of SEQ ID NO: 1613. In some embodiments, the polypeptide comprises the amino acid sequence of SEQ ID NO: 1614. In some embodiments, the polypeptide comprises the amino acid sequence of SEQ ID NO: 1615. In some embodiments, the polypeptide comprises the amino acid sequence of SEQ ID NO: 1616. In some embodiments, the polypeptide comprises the amino acid sequence of SEQ ID NO: 1617. In some embodiments, the polypeptide comprises the amino acid sequence of SEQ ID NO: 1618. In some embodiments, the polypeptide comprises the amino acid sequence of SEQ ID NO: 1619. In some embodiments, the polypeptide comprises the amino acid sequence of SEQ ID NO: 1620. In some embodiments, the polypeptide comprises the amino acid sequence of SEQ ID NO: 1621. In some embodiments, the polypeptide comprises the amino acid sequence of SEQ ID NO: 1622. In some embodiments, the polypeptide comprises the amino acid sequence of SEQ ID NO: 1623. In some embodiments, the polypeptide comprises the amino acid sequence of SEQ ID NO: 1624. In some embodiments, the polypeptide comprises the amino acid sequence of SEQ ID NO: 1625. In some embodiments, the polypeptide comprises the amino acid sequence of SEQ ID NO: 1626. In some embodiments, the polypeptide comprises the amino acid sequence of SEQ ID NO: 1627.

In some embodiments, the polypeptide comprises the amino acid sequence of SEQ ID NO: 1747. In some embodiments, the polypeptide comprises the amino acid sequence of SEQ ID NO: 1748. In some embodiments, the polypeptide comprises the amino acid sequence of SEQ ID NO: 1749. In some embodiments, the polypeptide comprises the amino acid sequence of SEQ ID NO: 1750. In some embodiments, the polypeptide comprises the amino acid sequence of SEQ ID NO: 1751. In some embodiments, the polypeptide comprises the amino acid sequence of SEQ ID NO: 1752. In some embodiments, the polypeptide comprises the amino acid sequence of SEQ ID NO: 1753. In some embodiments, the polypeptide comprises the amino acid sequence of SEQ ID NO: 1754. In some embodiments, the polypeptide comprises the amino acid sequence of SEQ ID NO: 1755. In some embodiments, the polypeptide comprises the amino acid sequence of SEQ ID NO: 1756. In some embodiments, the polypeptide comprises the amino acid sequence of SEQ ID NO: 1757. In some embodiments, the polypeptide comprises the amino acid sequence of SEQ ID NO: 1758. In some embodiments, the polypeptide comprises the amino acid sequence of SEQ ID NO: 1759. In some embodiments, the polypeptide comprises the amino acid sequence of SEQ ID NO: 1760. In some embodiments, the polypeptide comprises the amino acid sequence of SEQ ID NO: 1761. In some embodiments, the polypeptide comprises the amino acid sequence of SEQ ID NO: 1762. In some embodiments, the polypeptide comprises the amino acid sequence of SEQ ID NO: 1763. In some embodiments, the polypeptide comprises the amino acid sequence of SEQ ID NO: 1764. In some embodiments, the polypeptide comprises the amino acid sequence of SEQ ID NO: 1765. In some embodiments, the polypeptide comprises the amino acid sequence of SEQ ID NO: 1766. In some embodiments, the polypeptide comprises the amino acid sequence of SEQ ID NO: 1767. In some embodiments, the polypeptide comprises the amino acid sequence of SEQ ID NO: 1768. In some embodiments, the polypeptide comprises the amino acid sequence of SEQ ID NO: 1769. In some embodiments, the polypeptide comprises the amino acid sequence of SEQ ID NO: 1770. In some embodiments, the polypeptide comprises the amino acid sequence of SEQ ID NO: 1771. In some embodiments, the polypeptide comprises the amino acid sequence of SEQ ID NO: 1772. In some embodiments, the polypeptide comprises the amino acid sequence of SEQ ID NO: 1773. In some embodiments, the polypeptide comprises the amino acid sequence of SEQ ID NO: 1774. In some embodiments, the polypeptide comprises the amino acid sequence of SEQ ID NO: 1775. In some embodiments, the polypeptide comprises the amino acid sequence of SEQ ID NO: 1776. In some embodiments, the polypeptide comprises the amino acid sequence of SEQ ID NO: 1777. In some embodiments, the polypeptide comprises the amino acid sequence of SEQ ID NO: 1778. In some embodiments, the polypeptide comprises the amino acid sequence of SEQ ID NO: 1779. In some embodiments, the polypeptide comprises the amino acid sequence of SEQ ID NO: 1780. In some embodiments, the polypeptide comprises the amino acid sequence of SEQ ID NO: 1781. In some embodiments, the polypeptide comprises the amino acid sequence of SEQ ID NO: 1782. In some embodiments, the polypeptide comprises the amino acid sequence of SEQ ID NO: 1783. In some embodiments, the polypeptide comprises the amino acid sequence of SEQ ID NO: 1784. In some embodiments, the polypeptide comprises the amino acid sequence of SEQ ID NO: 1785. In some embodiments, the polypeptide comprises the amino acid sequence of SEQ ID NO: 1786. In some embodiments, the polypeptide comprises the amino acid sequence of SEQ ID NO: 1787. In some embodiments, the polypeptide comprises the amino acid sequence of SEQ ID NO: 1788. In some embodiments, the polypeptide comprises the amino acid sequence of SEQ ID NO: 1789. In some embodiments, the polypeptide comprises the amino acid sequence of SEQ ID NO: 1790. In some embodiments, the polypeptide comprises the amino acid sequence of SEQ ID NO: 1791. In some embodiments, the polypeptide comprises the amino acid sequence of SEQ ID NO: 1792. In some embodiments, the polypeptide comprises the amino acid sequence of SEQ ID NO: 1793. In some embodiments, the polypeptide comprises the amino acid sequence of SEQ ID NO: 1794. In some embodiments, the polypeptide comprises the amino acid sequence of SEQ ID NO: 1795. In some embodiments, the polypeptide comprises the amino acid sequence of SEQ ID NO: 1796. In some embodiments, the polypeptide comprises the amino acid sequence of SEQ ID NO: 1797. In some embodiments, the polypeptide comprises the amino acid sequence of SEQ ID NO: 1798. In some embodiments, the polypeptide comprises the amino acid sequence of SEQ ID NO: 1799. In some embodiments, the polypeptide comprises the amino acid sequence of SEQ ID NO: 1800. In some embodiments, the polypeptide comprises the amino acid sequence of SEQ ID NO: 1801. In some embodiments, the polypeptide comprises the amino acid sequence of SEQ ID NO: 1802. In some embodiments, the polypeptide comprises the amino acid sequence of SEQ ID NO: 1803. In some embodiments, the polypeptide comprises the amino acid sequence of SEQ ID NO: 1804. In some embodiments, the polypeptide comprises the amino acid sequence of SEQ ID NO: 1805. In some embodiments, the polypeptide comprises the amino acid sequence of SEQ ID NO: 1806. In some embodiments, the polypeptide comprises the amino acid sequence of SEQ ID NO: 1807. In some embodiments, the polypeptide comprises the amino acid sequence of SEQ ID NO: 1808. In some embodiments, the polypeptide comprises the amino acid sequence of SEQ ID NO: 1809. In some embodiments, the polypeptide comprises the amino acid sequence of SEQ ID NO: 1810. In some embodiments, the polypeptide comprises the amino acid sequence of SEQ ID NO: 1811. In some embodiments, the polypeptide comprises the amino acid sequence of SEQ ID NO: 1812. In some embodiments, the polypeptide comprises the amino acid sequence of SEQ ID NO: 1813. In some embodiments, the polypeptide comprises the amino acid sequence of SEQ ID NO: 1814. In some embodiments, the polypeptide comprises the amino acid sequence of SEQ ID NO: 1815. In some embodiments, the polypeptide comprises the amino acid sequence of SEQ ID NO: 1816. In some embodiments, the polypeptide comprises the amino acid sequence of SEQ ID NO: 1817. In some embodiments, the polypeptide comprises the amino acid sequence of SEQ ID NO: 1818. In some embodiments, the polypeptide comprises the amino acid sequence of SEQ ID NO: 1819. In some embodiments, the polypeptide comprises the amino acid sequence of SEQ ID NO: 1820. In some embodiments, the polypeptide comprises the amino acid sequence of SEQ ID NO: 1821. In some embodiments, the polypeptide comprises the amino acid sequence of SEQ ID NO: 1822. In some embodiments, the polypeptide comprises the amino acid sequence of SEQ ID NO: 1823. In some embodiments, the polypeptide comprises the amino acid sequence of SEQ ID NO: 1824. In some embodiments, the polypeptide comprises the amino acid sequence of SEQ ID NO: 1825. In some embodiments, the polypeptide comprises the amino acid sequence of SEQ ID NO: 1826. In some embodiments, the polypeptide comprises the amino acid sequence of SEQ ID NO: 1827. In some embodiments, the polypeptide comprises the amino acid sequence of SEQ ID NO: 1828. In some embodiments, the polypeptide comprises the amino acid sequence of SEQ ID NO: 1829. In some embodiments, the polypeptide comprises the amino acid sequence of SEQ ID NO: 1830. In some embodiments, the polypeptide comprises the amino acid sequence of SEQ ID NO: 1831. In some embodiments, the polypeptide comprises the amino acid sequence of SEQ ID NO: 1832. In some embodiments, the polypeptide comprises the amino acid sequence of SEQ ID NO: 1833. In some embodiments, the polypeptide comprises the amino acid sequence of SEQ ID NO: 1834. In some embodiments, the polypeptide comprises the amino acid sequence of SEQ ID NO: 1835. In some embodiments, the polypeptide comprises the amino acid sequence of SEQ ID NO: 1836. In some embodiments, the polypeptide comprises the amino acid sequence of SEQ ID NO: 1837. In some embodiments, the polypeptide comprises the amino acid sequence of SEQ ID NO: 1838. In some embodiments, the polypeptide comprises the amino acid sequence of SEQ ID NO: 1839. In some embodiments, the polypeptide comprises the amino acid sequence of SEQ ID NO: 1840. In some embodiments, the polypeptide comprises the amino acid sequence of SEQ ID NO: 1859. In some embodiments, the polypeptide comprises the amino acid sequence of SEQ ID NO: 1860. In some embodiments, the polypeptide comprises the amino acid sequence of SEQ ID NO: 1861. In some embodiments, the polypeptide comprises the amino acid sequence of SEQ ID NO: 1862. In some embodiments, the polypeptide comprises the amino acid sequence of SEQ ID NO: 1879. In some embodiments, the polypeptide comprises the amino acid sequence of SEQ ID NO: 1880. In some embodiments, the polypeptide comprises the amino acid sequence of SEQ ID NO: 1881.

In some embodiments, the polypeptide comprises the amino acid sequence of any one of SEQ ID NOs: 1587, 1592, 1615, 1626, 1818, 1822, 1825, or 1826. In some embodiments, the polypeptide comprises the amino acid sequence of any one of SEQ ID NOs: 1587, 1592, 1615, or 1626.

In some embodiments, the polypeptide comprises the amino acid sequence of any one of SEQ ID NOs: 1596, 1597, 1615, 1626, 1818, 1822, 1825, or 1826. In some embodiments, the polypeptide comprises the amino acid sequence of any one of SEQ ID NOs: 1626, 1818, 1822, 1825, or 1826. In some embodiments, the polypeptide comprises the amino acid sequence of any one of SEQ ID NOs: 1596, 1597, 1615, or 1626.

In some embodiments, the polypeptide consists of the amino acid sequence of any one of SEQ ID NOs: 1587-1627 or 1747. In some embodiments, the polypeptide consists of the amino acid sequence of any one of SEQ ID NOs: 1748-1840, 1859-1862, or 1879-1881.

In some embodiments, the polypeptide consists of the amino acid sequence of any one of SEQ ID NOs: 1587-1627.

In some embodiments, the polypeptide consists of the amino acid sequence of SEQ ID NO: 1587. In some embodiments, the polypeptide consists of the amino acid sequence of SEQ ID NO: 1588. In some embodiments, the polypeptide consists of the amino acid sequence of SEQ ID NO: 1589. In some embodiments, the polypeptide consists of the amino acid sequence of SEQ ID NO: 1590. In some embodiments, the polypeptide consists of the amino acid sequence of SEQ ID NO: 1591. In some embodiments, the polypeptide consists of the amino acid sequence of SEQ ID NO: 1592. In some embodiments, the polypeptide consists of the amino acid sequence of SEQ ID NO: 1593. In some embodiments, the polypeptide consists of the amino acid sequence of SEQ ID NO: 1594. In some embodiments, the polypeptide consists of the amino acid sequence of SEQ ID NO: 1595. In some embodiments, the polypeptide consists of the amino acid sequence of SEQ ID NO: 1596. In some embodiments, the polypeptide consists of the amino acid sequence of SEQ ID NO: 1597. In some embodiments, the polypeptide consists of the amino acid sequence of SEQ ID NO: 1598. In some embodiments, the polypeptide consists of the amino acid sequence of SEQ ID NO: 1599. In some embodiments, the polypeptide consists of the amino acid sequence of SEQ ID NO: 1600. In some embodiments, the polypeptide consists of the amino acid sequence of SEQ ID NO: 1601. In some embodiments, the polypeptide consists of the amino acid sequence of SEQ ID NO: 1602. In some embodiments, the polypeptide consists of the amino acid sequence of SEQ ID NO: 1603. In some embodiments, the polypeptide consists of the amino acid sequence of SEQ ID NO: 1604. In some embodiments, the polypeptide consists of the amino acid sequence of SEQ ID NO: 1605. In some embodiments, the polypeptide consists of the amino acid sequence of SEQ ID NO: 1606. In some embodiments, the polypeptide consists of the amino acid sequence of SEQ ID NO: 1607. In some embodiments, the polypeptide consists of the amino acid sequence of SEQ ID NO: 1608. In some embodiments, the polypeptide consists of the amino acid sequence of SEQ ID NO: 1609. In some embodiments, the polypeptide consists of the amino acid sequence of SEQ ID NO: 1610. In some embodiments, the polypeptide consists of the amino acid sequence of SEQ ID NO: 1611. In some embodiments, the polypeptide consists of the amino acid sequence of SEQ ID NO: 1612. In some embodiments, the polypeptide consists of the amino acid sequence of SEQ ID NO: 1613. In some embodiments, the polypeptide consists of the amino acid sequence of SEQ ID NO: 1614. In some embodiments, the polypeptide consists of the amino acid sequence of SEQ ID NO: 1615. In some embodiments, the polypeptide consists of the amino acid sequence of SEQ ID NO: 1616. In some embodiments, the polypeptide consists of the amino acid sequence of SEQ ID NO: 1617. In some embodiments, the polypeptide consists of the amino acid sequence of SEQ ID NO: 1618. In some embodiments, the polypeptide consists of the amino acid sequence of SEQ ID NO: 1619. In some embodiments, the polypeptide consists of the amino acid sequence of SEQ ID NO: 1620. In some embodiments, the polypeptide consists of the amino acid sequence of SEQ ID NO: 1621. In some embodiments, the polypeptide consists of the amino acid sequence of SEQ ID NO: 1622. In some embodiments, the polypeptide consists of the amino acid sequence of SEQ ID NO: 1623. In some embodiments, the polypeptide consists of the amino acid sequence of SEQ ID NO: 1624. In some embodiments, the polypeptide consists of the amino acid sequence of SEQ ID NO: 1625. In some embodiments, the polypeptide consists of the amino acid sequence of SEQ ID NO: 1626. In some embodiments, the polypeptide consists of the amino acid sequence of SEQ ID NO: 1627.

In some embodiments, the polypeptide consists of the amino acid sequence of SEQ ID NO: 1747. In some embodiments, the polypeptide consists of the amino acid sequence of SEQ ID NO: 1748. In some embodiments, the polypeptide consists of the amino acid sequence of SEQ ID NO: 1749. In some embodiments, the polypeptide consists of the amino acid sequence of SEQ ID NO: 1750. In some embodiments, the polypeptide consists of the amino acid sequence of SEQ ID NO: 1751. In some embodiments, the polypeptide consists of the amino acid sequence of SEQ ID NO: 1752. In some embodiments, the polypeptide consists of the amino acid sequence of SEQ ID NO: 1753. In some embodiments, the polypeptide consists of the amino acid sequence of SEQ ID NO: 1754. In some embodiments, the polypeptide consists of the amino acid sequence of SEQ ID NO: 1755. In some embodiments, the polypeptide consists of the amino acid sequence of SEQ ID NO: 1756. In some embodiments, the polypeptide consists of the amino acid sequence of SEQ ID NO: 1757. In some embodiments, the polypeptide consists of the amino acid sequence of SEQ ID NO: 1758. In some embodiments, the polypeptide consists of the amino acid sequence of SEQ ID NO: 1759. In some embodiments, the polypeptide consists of the amino acid sequence of SEQ ID NO: 1760. In some embodiments, the polypeptide consists of the amino acid sequence of SEQ ID NO: 1761. In some embodiments, the polypeptide consists of the amino acid sequence of SEQ ID NO: 1762. In some embodiments, the polypeptide consists of the amino acid sequence of SEQ ID NO: 1763. In some embodiments, the polypeptide consists of the amino acid sequence of SEQ ID NO: 1764. In some embodiments, the polypeptide consists of the amino acid sequence of SEQ ID NO: 1765. In some embodiments, the polypeptide consists of the amino acid sequence of SEQ ID NO: 1766. In some embodiments, the polypeptide consists of the amino acid sequence of SEQ ID NO: 1767. In some embodiments, the polypeptide consists of the amino acid sequence of SEQ ID NO: 1768. In some embodiments, the polypeptide consists of the amino acid sequence of SEQ ID NO: 1769. In some embodiments, the polypeptide consists of the amino acid sequence of SEQ ID NO: 1770. In some embodiments, the polypeptide consists of the amino acid sequence of SEQ ID NO: 1771. In some embodiments, the polypeptide consists of the amino acid sequence of SEQ ID NO: 1772. In some embodiments, the polypeptide consists of the amino acid sequence of SEQ ID NO: 1773. In some embodiments, the polypeptide consists of the amino acid sequence of SEQ ID NO: 1774. In some embodiments, the polypeptide consists of the amino acid sequence of SEQ ID NO: 1775. In some embodiments, the polypeptide consists of the amino acid sequence of SEQ ID NO: 1776. In some embodiments, the polypeptide consists of the amino acid sequence of SEQ ID NO: 1777. In some embodiments, the polypeptide consists of the amino acid sequence of SEQ ID NO: 1778. In some embodiments, the polypeptide consists of the amino acid sequence of SEQ ID NO: 1779. In some embodiments, the polypeptide consists of the amino acid sequence of SEQ ID NO: 1780. In some embodiments, the polypeptide consists of the amino acid sequence of SEQ ID NO: 1781. In some embodiments, the polypeptide consists of the amino acid sequence of SEQ ID NO: 1782. In some embodiments, the polypeptide consists of the amino acid sequence of SEQ ID NO: 1783. In some embodiments, the polypeptide consists of the amino acid sequence of SEQ ID NO: 1784. In some embodiments, the polypeptide consists of the amino acid sequence of SEQ ID NO: 1785. In some embodiments, the polypeptide consists of the amino acid sequence of SEQ ID NO: 1786. In some embodiments, the polypeptide consists of the amino acid sequence of SEQ ID NO: 1787. In some embodiments, the polypeptide consists of the amino acid sequence of SEQ ID NO: 1788. In some embodiments, the polypeptide consists of the amino acid sequence of SEQ ID NO: 1789. In some embodiments, the polypeptide consists of the amino acid sequence of SEQ ID NO: 1790. In some embodiments, the polypeptide consists of the amino acid sequence of SEQ ID NO: 1791. In some embodiments, the polypeptide consists of the amino acid sequence of SEQ ID NO: 1792. In some embodiments, the polypeptide consists of the amino acid sequence of SEQ ID NO: 1793. In some embodiments, the polypeptide consists of the amino acid sequence of SEQ ID NO: 1794. In some embodiments, the polypeptide consists of the amino acid sequence of SEQ ID NO: 1795. In some embodiments, the polypeptide consists of the amino acid sequence of SEQ ID NO: 1796. In some embodiments, the polypeptide consists of the amino acid sequence of SEQ ID NO: 1797. In some embodiments, the polypeptide consists of the amino acid sequence of SEQ ID NO: 1798. In some embodiments, the polypeptide consists of the amino acid sequence of SEQ ID NO: 1799. In some embodiments, the polypeptide consists of the amino acid sequence of SEQ ID NO: 1800. In some embodiments, the polypeptide consists of the amino acid sequence of SEQ ID NO: 1801. In some embodiments, the polypeptide consists of the amino acid sequence of SEQ ID NO: 1802. In some embodiments, the polypeptide consists of the amino acid sequence of SEQ ID NO: 1803. In some embodiments, the polypeptide consists of the amino acid sequence of SEQ ID NO: 1804. In some embodiments, the polypeptide consists of the amino acid sequence of SEQ ID NO: 1805. In some embodiments, the polypeptide consists of the amino acid sequence of SEQ ID NO: 1806. In some embodiments, the polypeptide consists of the amino acid sequence of SEQ ID NO: 1807. In some embodiments, the polypeptide consists of the amino acid sequence of SEQ ID NO: 1808. In some embodiments, the polypeptide consists of the amino acid sequence of SEQ ID NO: 1809. In some embodiments, the polypeptide consists of the amino acid sequence of SEQ ID NO: 1810. In some embodiments, the polypeptide consists of the amino acid sequence of SEQ ID NO: 1811. In some embodiments, the polypeptide consists of the amino acid sequence of SEQ ID NO: 1812. In some embodiments, the polypeptide consists of the amino acid sequence of SEQ ID NO: 1813. In some embodiments, the polypeptide consists of the amino acid sequence of SEQ ID NO: 1814. In some embodiments, the polypeptide consists of the amino acid sequence of SEQ ID NO: 1815. In some embodiments, the polypeptide consists of the amino acid sequence of SEQ ID NO: 1816. In some embodiments, the polypeptide consists of the amino acid sequence of SEQ ID NO: 1817. In some embodiments, the polypeptide consists of the amino acid sequence of SEQ ID NO: 1818. In some embodiments, the polypeptide consists of the amino acid sequence of SEQ ID NO: 1819. In some embodiments, the polypeptide consists of the amino acid sequence of SEQ ID NO: 1820. In some embodiments, the polypeptide consists of the amino acid sequence of SEQ ID NO: 1821. In some embodiments, the polypeptide consists of the amino acid sequence of SEQ ID NO: 1822. In some embodiments, the polypeptide consists of the amino acid sequence of SEQ ID NO: 1823. In some embodiments, the polypeptide consists of the amino acid sequence of SEQ ID NO: 1824. In some embodiments, the polypeptide consists of the amino acid sequence of SEQ ID NO: 1825. In some embodiments, the polypeptide consists of the amino acid sequence of SEQ ID NO: 1826. In some embodiments, the polypeptide consists of the amino acid sequence of SEQ ID NO: 1827. In some embodiments, the polypeptide consists of the amino acid sequence of SEQ ID NO: 1828. In some embodiments, the polypeptide consists of the amino acid sequence of SEQ ID NO: 1829. In some embodiments, the polypeptide consists of the amino acid sequence of SEQ ID NO: 1830. In some embodiments, the polypeptide consists of the amino acid sequence of SEQ ID NO: 1831. In some embodiments, the polypeptide consists of the amino acid sequence of SEQ ID NO: 1832. In some embodiments, the polypeptide consists of the amino acid sequence of SEQ ID NO: 1833. In some embodiments, the polypeptide consists of the amino acid sequence of SEQ ID NO: 1834. In some embodiments, the polypeptide consists of the amino acid sequence of SEQ ID NO: 1835. In some embodiments, the polypeptide consists of the amino acid sequence of SEQ ID NO: 1836. In some embodiments, the polypeptide consists of the amino acid sequence of SEQ ID NO: 1837. In some embodiments, the polypeptide consists of the amino acid sequence of SEQ ID NO: 1838. In some embodiments, the polypeptide consists of the amino acid sequence of SEQ ID NO: 1839. In some embodiments, the polypeptide consists of the amino acid sequence of SEQ ID NO: 1840. In some embodiments, the polypeptide consists of the amino acid sequence of SEQ ID NO: 1859. In some embodiments, the polypeptide consists of the amino acid sequence of SEQ ID NO: 1860. In some embodiments, the polypeptide consists of the amino acid sequence of SEQ ID NO: 1861. In some embodiments, the polypeptide consists of the amino acid sequence of SEQ ID NO: 1862. In some embodiments, the polypeptide consists of the amino acid sequence of SEQ ID NO: 1879. In some embodiments, the polypeptide consists of the amino acid sequence of SEQ ID NO: 1880. In some embodiments, the polypeptide consists of the amino acid sequence of SEQ ID NO: 1881.

In some embodiments, the polypeptide consists of the amino acid sequence of any one of SEQ ID NOs: 1587, 1592, 1615, 1626, 1818, 1822, 1825, or 1826. In some embodiments, the polypeptide consists of the amino acid sequence of any one of SEQ ID NOs: 1587, 1592, 1615, or 1626. In some embodiments, the polypeptide comprises the amino acid sequence of any one of SEQ ID NOs: 1626, 1818, 1822, 1825, or 1826.

In some embodiments, the polypeptide consists of the amino acid sequence of any one of SEQ ID NOs: 1596, 1597, 1615, 1626, 1818, 1822, 1825, or 1826. In some embodiments, the polypeptide consists of the amino acid sequence of any one of SEQ ID NOs: 1596, 1597, 1615, or 1626.

In some embodiments, the polypeptide agonizes human GCGR (“hGCGR”). In some embodiments, the hGCGR comprises the amino acid sequence of SEQ ID NO: 1576.

In some embodiments, the polypeptide has a hGCGR EC₅₀ of less than or equal to 1 nM.

In some embodiments, the polypeptide has a hGCGR EC₅₀ of less than or equal to 500 pM (e.g., less than or equal to 475 pM, less than or equal to 450 pM, less than or equal to 425 pM, less than or equal to 400 pM, less than or equal to 375 pM, less than or equal to 350 pM, less than or equal to 325 pM, less than or equal to 300 pM, less than or equal to 275 pM, less than or equal to 250 pM, less than or equal to 225 pM, less than or equal to 200 pM, less than or equal to 175 pM, less than or equal to 150 pM, less than or equal to 125 pM, less than or equal to 100 pM). In some embodiments, the hGCGR EC₅₀ is measured in a functional assay described in “EXAMPLE 1: GCG Receptor Agonist Activity.”

In some embodiments, the polypeptide has a hGCGR EC₅₀ of 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 155 pM, 200 pM, 205 pM, 210 pM, 215 pM, 220 pM, 225 pM, 230 pM, 235 pM, 240 pM, 245 pM, 250 pM, 255 pM, 260 pM, 265 pM, 270 pM, 275 pM, 280 pM, 285 pM, 290 pM, 295 pM, 300 pM, 305 pM, 310 pM, 315 pM, 320 pM, 325 pM, 330 pM, 335 pM, 340 pM, 345 pM, 350 pM, 355 pM, 360 pM, 365 pM, 370 pM, 375 pM, 380 pM, 385 pM, 390 pM, 395 pM, 400 pM, 405 pM, 410 pM, 415 pM, 420 pM, 425 pM, 430 pM, 435 pM, 440 pM, 445 pM, 450 pM, 455 pM, 460 pM, 465 pM, 470 pM, 475 pM, 480 pM, 485 pM, 490 pM, 495 pM, or 500 pM. In some embodiments, the hGCGR EC₅₀ is measured in a functional assay described in “EXAMPLE 1: GCG Receptor Agonist Activity.”

In some embodiments, the polypeptide has a human glucagon-like peptide-1 receptor (“hGLP-1R”) EC₅₀:hGCGR EC₅₀ ratio of at least 30:1. In some embodiments, the polypeptide has a hGLP-1R EC₅₀:hGCGR EC₅₀ ratio of at least 40:1. In some embodiments, the polypeptide has a hGLP-1R EC₅₀:hGCGR EC₅₀ ratio of at least 50:1. In some embodiments, the polypeptide has a hGLP-1R EC₅₀:hGCGR EC₅₀ ratio of at least 60:1. In some embodiments, the polypeptide has a hGLP-1R EC₅₀:hGCGR EC₅₀ ratio of at least 70:1. In some embodiments, the polypeptide has a hGLP-1R EC₅₀:hGCGR EC₅₀ ratio of at least 80:1. In some embodiments, the polypeptide has a hGLP-1R EC₅₀:hGCGR EC₅₀ ratio of at least 90:1. In some embodiments, the polypeptide has a hGLP-1R EC₅₀:hGCGR EC₅₀ ratio of at least 100:1. In some embodiments, the polypeptide has a hGLP-1R EC₅₀:hGCGR EC₅₀ ratio of at least 150:1. In some embodiments, the polypeptide has a hGLP-1R EC₅₀:hGCGR EC₅₀ ratio of at least 200:1. In some embodiments, the polypeptide has a hGLP-1R EC₅₀:hGCGR EC₅₀ ratio of at least 250:1. In some embodiments, the polypeptide has a hGLP-1R EC₅₀:hGCGR EC₅₀ ratio of at least 300:1. In some embodiments, the polypeptide has a hGLP-1R EC₅₀:hGCGR EC₅₀ ratio of at least 350:1. In some embodiments, the polypeptide has a hGLP-1R EC₅₀:hGCGR EC₅₀ ratio of at least 400:1. In some embodiments, the polypeptide has a hGLP-1R EC₅₀:hGCGR EC₅₀ ratio of at least 450:1. In some embodiments, the polypeptide has a hGLP-1R EC₅₀:hGCGR EC₅₀ ratio of at least 500:1. In some embodiments, the polypeptide has a hGLP-1R EC₅₀:hGCGR EC₅₀ ratio of at least 550:1. In some embodiments, the polypeptide has a hGLP-1R EC₅₀:hGCGR EC₅₀ ratio of at least 650:1. In some embodiments, the polypeptide has a hGLP-1R EC₅₀:hGCGR EC₅₀ ratio of at least 700:1. In some embodiments, the polypeptide has a hGLP-1R EC₅₀:hGCGR EC₅₀ ratio of at least 750:1. In some embodiments, the polypeptide has a hGLP-1R EC₅₀:hGCGR EC₅₀ ratio of at least 800:1. In some embodiments, the polypeptide has a hGLP-1R EC₅₀:hGCGR EC₅₀ ratio of at least 850:1. In some embodiments, the polypeptide has a hGLP-1R EC₅₀:hGCGR EC₅₀ ratio of at least 900:1. In some embodiments, the polypeptide has a hGLP-1R EC₅₀:hGCGR EC₅₀ ratio of at least 950:1. In some embodiments, the polypeptide has a hGLP-1R EC₅₀:hGCGR EC₅₀ ratio of at least 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

In some embodiments, the polypeptide has a hGLP-1R EC₅₀:hGCGR EC₅₀ ratio of 30:1, 40:1, 45:1, 50:1, 55:1, 60:1, 65:1, 70:1, 75:1, 80:1, 85:1, 90:1, 95:1, 100:1, 125:1, 150:1, 175:1, 200:1, 225:1, 250:1, 275:1, 300:1, 325:1, 350:1, 375:1, 400:1, 425:1, 450:1, 475:1, 500:1, 525:1, 550:1, 575:1, 600:1, 625:1, 650:1, 675:1, 700:1, 725:1, 750:1, 775:1, 800:1, 825:1, 850:1, 875:1, 900:1, 925:1, 950:1, 975:1, or 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

The present disclosure further provides molecules in which a polypeptide agonist disclosed herein is conjugated to a linker moiety, such as, e.g., a linker polypeptide. Linker moieties, including linker polypeptides, are discussed in more detail below. Such linker moieties include, for example, the linker sequences of any one of SEQ ID NOs: 1628-1683, 1739-1746, or 1841-1852. If desired, such molecules can be further conjugated to another molecule via the linker moiety. To facilitate such conjugation reactions, the polypeptide agonist may be derivatized.

For example, provided herein is a molecule comprising a first polypeptide that agonizes a glucagon receptor (“GCGR”) selected from the polypeptides provided herein (e.g., polypeptides comprising the amino acid sequence of any one of SEQ ID NOs: 1587-1627, 1747-1840, 1859-1862, or 1879-1881; SEQ ID NOs: 1587-1627 or 1747; SEQ ID NOs: 1587-1627; SEQ ID NOs: 1748-1840, 1859-1862, or 1879-1881); and a second polypeptide comprising the amino acid sequence of any one of SEQ ID NOs: 1628-1683, 1739-1746, or 1841-1852 (e.g., SEQ ID NOs: 1587-1627, 1747-1749, 1751-1792, 1794-1798, 1801-1830, 1833-1840, 1859-1862, or 1879-1881), wherein the C-terminus of the second polypeptide is covalently linked to an ε-amino group of a lysine residue of the first polypeptide, such as, e.g., a molecule comprising a first polypeptide that agonizes a glucagon receptor (“GCGR”) selected from the polypeptides provided herein (e.g., polypeptides comprising the amino acid sequence of any one of SEQ ID NOs: 1587-1627, 1747-1840, 1859-1862, or 1879-1881; SEQ ID NOs: 1587-1627 or 1747; SEQ ID NOs: 1587-1627; SEQ ID NOs: 1748-1840, 1859-1862, or 1879-1881); and a second polypeptide comprising the amino acid sequence of any one of SEQ ID NOs: 1628-1683, wherein the C-terminus of the second polypeptide is covalently linked to an ε-amino group of a lysine residue of the first polypeptide. For example, in some embodiments, a lysine residue of the first polypeptide and the C-terminus of the second polypeptide are covalently linked by an amide bond.

Illustratively, in some embodiments, the first polypeptide and the second polypeptide are covalently linked by an amide bond formed by the condensation of an ε-amino group of a lysine residue of the first polypeptide and a carboxyl group of the C-terminus of the second polypeptide.

In some embodiments, the molecule is a branched polypeptide. In some embodiments, the molecule is a branched peptide.

In some embodiments, the C-terminus of the second polypeptide is covalently linked to the ε-amino group of a lysine at position 21, position 24, position 28, or position 31 of the first polypeptide (e.g., a lysine residue at position 21, position 24, position 28, or position 31 of the first polypeptide is covalently linked to the C-terminus of the second polypeptide by an amide bond, e.g., an amide bond formed by the condensation of an ε-amino group of a lysine residue of the first polypeptide and a carboxyl group of the C-terminus of the second polypeptide). In some embodiments, the C-terminus of the second polypeptide is covalently linked to the β-amino group of a lysine at position 24 or position 28 of the first polypeptide (e.g., a lysine residue at position 24 or position 28 of the first polypeptide is covalently linked to the C-terminus of the second polypeptide by an amide bond, e.g., an amide bond formed by the condensation of an ε-amino group of a lysine residue of the first polypeptide and a carboxyl group of the C-terminus of the second polypeptide). In some embodiments, the C-terminus of the second polypeptide is covalently linked to the ε-amino group of a lysine at position 21 of the first polypeptide. In some embodiments, the C-terminus of the second polypeptide is covalently linked to the ε-amino group of a lysine at position 24 of the first polypeptide. In some embodiments, the C-terminus of the second polypeptide is covalently linked to the ε-amino group of a lysine at position 28 of the first polypeptide. In some embodiments, the C-terminus of the second polypeptide is covalently linked to the ε-amino group of a lysine at position 31 of the first polypeptide.

In some embodiments, the first polypeptide comprises at least 25 amino acids, wherein: the first polypeptide comprises 5-bromo-tryptophan at position 25; and the first polypeptide has at least 79% (e.g., at least 82%, at least 86%, at least 89%, at least 93%, at least 96%) sequence identity to the amino acid sequence of SEQ ID NO: 1587. In some embodiments, the first polypeptide comprises SEQ ID NO: 1587. In some embodiments, the first polypeptide consists of SEQ ID NO: 1587.

In some embodiments, the first polypeptide comprises at least 28 amino acids, wherein: the first polypeptide comprises 2-aminoisobutyric acid at position 16 and lysine, serine, or aspartic acid at position 28; and the first polypeptide has at least 89% (e.g., at least 93%, at least 96%) sequence identity to the amino acid sequence of SEQ ID NO: 1596. In some embodiments, the first polypeptide comprises SEQ ID NO: 1596. In some embodiments, the first polypeptide consists of SEQ ID NO: 1596.

In some embodiments, the first polypeptide comprises at least 28 amino acids, wherein: the first polypeptide comprises 2-aminoisobutyric acid at position 16, leucine at position 27, and lysine at position 28; and the first polypeptide has at least 89% (e.g., at least 93%, at least 96%) sequence identity to the amino acid sequence of SEQ ID NO: 1615. In some embodiments, the first polypeptide comprises SEQ ID NO: 1615. In some embodiments, the first polypeptide consists of SEQ ID NO: 1615.

In some embodiments, the first polypeptide comprises at least 28 amino acids, wherein: the first polypeptide comprises 2-aminoisobutyric acid at position 16, aspartic acid at position 24, and lysine at position 28; and the first polypeptide has at least 89% (e.g., at least 93%, at least 96%) sequence identity to the amino acid sequence of SEQ ID NO: 1626.

In some embodiments, the first polypeptide comprises SEQ ID NO: 1626. In some embodiments, the first polypeptide consists of SEQ ID NO: 1626.

In some embodiments, the first polypeptide comprises at least 28 amino acids, wherein the first polypeptide comprises a d-serine at position 2 and a 2-aminoisobutyric acid at position 16; and the first polypeptide has at least 89% (e.g., at least 93%, at least 96%) sequence identity to the amino acid sequence of SEQ ID NO: 1822. In some embodiments, the first polypeptide comprises SEQ ID NO: 1822. In some embodiments, the first polypeptide consists of SEQ ID NO: 1822.

In some embodiments, the first polypeptide comprises the amino acid sequence of any one of SEQ ID NOs: 1587-1627, 1747-1840, 1859-1862, or 1879-1881. In some embodiments, the first polypeptide comprises the amino acid sequence of any one of SEQ ID NOs: 1587-1627. In some embodiments, the first polypeptide comprises the amino acid sequence of any one of SEQ ID NOs: 1587-1627 or 1747. In some embodiments, the first polypeptide comprises the amino acid sequence of any one of SEQ ID NOs: 1748-1840, 1859-1862, or 1879-1881.

In some embodiments, the first polypeptide consists of the amino acid sequence of any one of SEQ ID NOs: 1587-1627, 1747-1840, 1859-1862, or 1879-1881. In some embodiments, the first polypeptide consists of the amino acid sequence of any one of SEQ ID NOs: 1587-1627. In some embodiments, the first polypeptide consists of the amino acid sequence of any one of SEQ ID NOs: 1587-1627 or 1747. In some embodiments, the first polypeptide consists of the amino acid sequence of any one of SEQ ID NOs: 1748-1840, 1859-1862, or 1879-1881.

In some embodiments, the first polypeptide comprises the amino acid sequence of SEQ ID NO: 1587. In some embodiments, the first polypeptide comprises the amino acid sequence of SEQ ID NO: 1588. In some embodiments, the first polypeptide comprises the amino acid sequence of SEQ ID NO: 1589. In some embodiments, the first polypeptide comprises the amino acid sequence of SEQ ID NO: 1590. In some embodiments, the first polypeptide comprises the amino acid sequence of SEQ ID NO: 1591. In some embodiments, the first polypeptide comprises the amino acid sequence of SEQ ID NO: 1592. In some embodiments, the first polypeptide comprises the amino acid sequence of SEQ ID NO: 1593.

In some embodiments, the first polypeptide comprises the amino acid sequence of SEQ ID NO: 1594. In some embodiments, the first polypeptide comprises the amino acid sequence of SEQ ID NO: 1595. In some embodiments, the first polypeptide comprises the amino acid sequence of SEQ ID NO: 1596. In some embodiments, the first polypeptide comprises the amino acid sequence of SEQ ID NO: 1597. In some embodiments, the first polypeptide comprises the amino acid sequence of SEQ ID NO: 1598. In some embodiments, the first polypeptide comprises the amino acid sequence of SEQ ID NO: 1599. In some embodiments, the first polypeptide comprises the amino acid sequence of SEQ ID NO: 1600. In some embodiments, the first polypeptide comprises the amino acid sequence of SEQ ID NO: 1601. In some embodiments, the first polypeptide comprises the amino acid sequence of SEQ ID NO: 1602. In some embodiments, the first polypeptide comprises the amino acid sequence of SEQ ID NO: 1603. In some embodiments, the first polypeptide comprises the amino acid sequence of SEQ ID NO: 1604. In some embodiments, the first polypeptide comprises the amino acid sequence of SEQ ID NO: 1605. In some embodiments, the first polypeptide comprises the amino acid sequence of SEQ ID NO: 1606. In some embodiments, the first polypeptide comprises the amino acid sequence of SEQ ID NO: 1607. In some embodiments, the first polypeptide comprises the amino acid sequence of SEQ ID NO: 1608. In some embodiments, the first polypeptide comprises the amino acid sequence of SEQ ID NO: 1609. In some embodiments, the first polypeptide comprises the amino acid sequence of SEQ ID NO: 1610. In some embodiments, the first polypeptide comprises the amino acid sequence of SEQ ID NO: 1611. In some embodiments, the first polypeptide comprises the amino acid sequence of SEQ ID NO: 1612. In some embodiments, the first polypeptide comprises the amino acid sequence of SEQ ID NO: 1613. In some embodiments, the first polypeptide comprises the amino acid sequence of SEQ ID NO: 1614. In some embodiments, the first polypeptide comprises the amino acid sequence of SEQ ID NO: 1615. In some embodiments, the first polypeptide comprises the amino acid sequence of SEQ ID NO: 1616. In some embodiments, the first polypeptide comprises the amino acid sequence of SEQ ID NO: 1617. In some embodiments, the first polypeptide comprises the amino acid sequence of SEQ ID NO: 1618. In some embodiments, the first polypeptide comprises the amino acid sequence of SEQ ID NO: 1619. In some embodiments, the first polypeptide comprises the amino acid sequence of SEQ ID NO: 1620. In some embodiments, the first polypeptide comprises the amino acid sequence of SEQ ID NO: 1621. In some embodiments, the first polypeptide comprises the amino acid sequence of SEQ ID NO: 1622. In some embodiments, the first polypeptide comprises the amino acid sequence of SEQ ID NO: 1623. In some embodiments, the first polypeptide comprises the amino acid sequence of SEQ ID NO: 1624. In some embodiments, the first polypeptide comprises the amino acid sequence of SEQ ID NO: 1625. In some embodiments, the first polypeptide comprises the amino acid sequence of SEQ ID NO: 1626. In some embodiments, the first polypeptide comprises the amino acid sequence of SEQ ID NO: 1627.

In some embodiments, the first polypeptide comprises the amino acid sequence of SEQ ID NO: 1747. In some embodiments, the first polypeptide comprises the amino acid sequence of SEQ ID NO: 1748. In some embodiments, the first polypeptide comprises the amino acid sequence of SEQ ID NO: 1749. In some embodiments, the first polypeptide comprises the amino acid sequence of SEQ ID NO: 1750. In some embodiments, the first polypeptide comprises the amino acid sequence of SEQ ID NO: 1751. In some embodiments, the first polypeptide comprises the amino acid sequence of SEQ ID NO: 1752. In some embodiments, the first polypeptide comprises the amino acid sequence of SEQ ID NO: 1753. In some embodiments, the first polypeptide comprises the amino acid sequence of SEQ ID NO: 1754. In some embodiments, the first polypeptide comprises the amino acid sequence of SEQ ID NO: 1755. In some embodiments, the first polypeptide comprises the amino acid sequence of SEQ ID NO: 1756. In some embodiments, the first polypeptide comprises the amino acid sequence of SEQ ID NO: 1757. In some embodiments, the first polypeptide comprises the amino acid sequence of SEQ ID NO: 1758. In some embodiments, the first polypeptide comprises the amino acid sequence of SEQ ID NO: 1759. In some embodiments, the first polypeptide comprises the amino acid sequence of SEQ ID NO: 1760. In some embodiments, the first polypeptide comprises the amino acid sequence of SEQ ID NO: 1761. In some embodiments, the first polypeptide comprises the amino acid sequence of SEQ ID NO: 1762. In some embodiments, the first polypeptide comprises the amino acid sequence of SEQ ID NO: 1763. In some embodiments, the first polypeptide comprises the amino acid sequence of SEQ ID NO: 1764. In some embodiments, the first polypeptide comprises the amino acid sequence of SEQ ID NO: 1765. In some embodiments, the first polypeptide comprises the amino acid sequence of SEQ ID NO: 1766. In some embodiments, the first polypeptide comprises the amino acid sequence of SEQ ID NO: 1767. In some embodiments, the first polypeptide comprises the amino acid sequence of SEQ ID NO: 1768. In some embodiments, the first polypeptide comprises the amino acid sequence of SEQ ID NO: 1769. In some embodiments, the first polypeptide comprises the amino acid sequence of SEQ ID NO: 1770. In some embodiments, the first polypeptide comprises the amino acid sequence of SEQ ID NO: 1771. In some embodiments, the first polypeptide comprises the amino acid sequence of SEQ ID NO: 1772. In some embodiments, the first polypeptide comprises the amino acid sequence of SEQ ID NO: 1773. In some embodiments, the first polypeptide comprises the amino acid sequence of SEQ ID NO: 1774. In some embodiments, the first polypeptide comprises the amino acid sequence of SEQ ID NO: 1775. In some embodiments, the first polypeptide comprises the amino acid sequence of SEQ ID NO: 1776. In some embodiments, the first polypeptide comprises the amino acid sequence of SEQ ID NO: 1777. In some embodiments, the first polypeptide comprises the amino acid sequence of SEQ ID NO: 1778. In some embodiments, the first polypeptide comprises the amino acid sequence of SEQ ID NO: 1779. In some embodiments, the first polypeptide comprises the amino acid sequence of SEQ ID NO: 1780. In some embodiments, the first polypeptide comprises the amino acid sequence of SEQ ID NO: 1781. In some embodiments, the first polypeptide comprises the amino acid sequence of SEQ ID NO: 1782. In some embodiments, the first polypeptide comprises the amino acid sequence of SEQ ID NO: 1783. In some embodiments, the first polypeptide comprises the amino acid sequence of SEQ ID NO: 1784. In some embodiments, the first polypeptide comprises the amino acid sequence of SEQ ID NO: 1785. In some embodiments, the first polypeptide comprises the amino acid sequence of SEQ ID NO: 1786. In some embodiments, the first polypeptide comprises the amino acid sequence of SEQ ID NO: 1787. In some embodiments, the first polypeptide comprises the amino acid sequence of SEQ ID NO: 1788. In some embodiments, the first polypeptide comprises the amino acid sequence of SEQ ID NO: 1789. In some embodiments, the first polypeptide comprises the amino acid sequence of SEQ ID NO: 1790. In some embodiments, the first polypeptide comprises the amino acid sequence of SEQ ID NO: 1791. In some embodiments, the first polypeptide comprises the amino acid sequence of SEQ ID NO: 1792. In some embodiments, the first polypeptide comprises the amino acid sequence of SEQ ID NO: 1793. In some embodiments, the first polypeptide comprises the amino acid sequence of SEQ ID NO: 1794. In some embodiments, the first polypeptide comprises the amino acid sequence of SEQ ID NO: 1795. In some embodiments, the first polypeptide comprises the amino acid sequence of SEQ ID NO: 1796. In some embodiments, the first polypeptide comprises the amino acid sequence of SEQ ID NO: 1797. In some embodiments, the first polypeptide comprises the amino acid sequence of SEQ ID NO: 1798. In some embodiments, the first polypeptide comprises the amino acid sequence of SEQ ID NO: 1799. In some embodiments, the first polypeptide comprises the amino acid sequence of SEQ ID NO: 1800. In some embodiments, the first polypeptide comprises the amino acid sequence of SEQ ID NO: 1801. In some embodiments, the first polypeptide comprises the amino acid sequence of SEQ ID NO: 1802. In some embodiments, the first polypeptide comprises the amino acid sequence of SEQ ID NO: 1803. In some embodiments, the first polypeptide comprises the amino acid sequence of SEQ ID NO: 1804. In some embodiments, the first polypeptide comprises the amino acid sequence of SEQ ID NO: 1805. In some embodiments, the first polypeptide comprises the amino acid sequence of SEQ ID NO: 1806. In some embodiments, the first polypeptide comprises the amino acid sequence of SEQ ID NO: 1807. In some embodiments, the first polypeptide comprises the amino acid sequence of SEQ ID NO: 1808. In some embodiments, the first polypeptide comprises the amino acid sequence of SEQ ID NO: 1809. In some embodiments, the first polypeptide comprises the amino acid sequence of SEQ ID NO: 1810. In some embodiments, the first polypeptide comprises the amino acid sequence of SEQ ID NO: 1811. In some embodiments, the first polypeptide comprises the amino acid sequence of SEQ ID NO: 1812. In some embodiments, the first polypeptide comprises the amino acid sequence of SEQ ID NO: 1813. In some embodiments, the first polypeptide comprises the amino acid sequence of SEQ ID NO: 1814. In some embodiments, the first polypeptide comprises the amino acid sequence of SEQ ID NO: 1815. In some embodiments, the first polypeptide comprises the amino acid sequence of SEQ ID NO: 1816. In some embodiments, the first polypeptide comprises the amino acid sequence of SEQ ID NO: 1817. In some embodiments, the first polypeptide comprises the amino acid sequence of SEQ ID NO: 1818. In some embodiments, the first polypeptide comprises the amino acid sequence of SEQ ID NO: 1819. In some embodiments, the first polypeptide comprises the amino acid sequence of SEQ ID NO: 1820. In some embodiments, the first polypeptide comprises the amino acid sequence of SEQ ID NO: 1821. In some embodiments, the first polypeptide comprises the amino acid sequence of SEQ ID NO: 1822. In some embodiments, the first polypeptide comprises the amino acid sequence of SEQ ID NO: 1823. In some embodiments, the first polypeptide comprises the amino acid sequence of SEQ ID NO: 1824. In some embodiments, the first polypeptide comprises the amino acid sequence of SEQ ID NO: 1825. In some embodiments, the first polypeptide comprises the amino acid sequence of SEQ ID NO: 1826. In some embodiments, the first polypeptide comprises the amino acid sequence of SEQ ID NO: 1827. In some embodiments, the first polypeptide comprises the amino acid sequence of SEQ ID NO: 1828. In some embodiments, the first polypeptide comprises the amino acid sequence of SEQ ID NO: 1829. In some embodiments, the first polypeptide comprises the amino acid sequence of SEQ ID NO: 1830. In some embodiments, the first polypeptide comprises the amino acid sequence of SEQ ID NO: 1831. In some embodiments, the first polypeptide comprises the amino acid sequence of SEQ ID NO: 1832. In some embodiments, the first polypeptide comprises the amino acid sequence of SEQ ID NO: 1833. In some embodiments, the first polypeptide comprises the amino acid sequence of SEQ ID NO: 1834. In some embodiments, the first polypeptide comprises the amino acid sequence of SEQ ID NO: 1835. In some embodiments, the first polypeptide comprises the amino acid sequence of SEQ ID NO: 1836. In some embodiments, the first polypeptide comprises the amino acid sequence of SEQ ID NO: 1837. In some embodiments, the first polypeptide comprises the amino acid sequence of SEQ ID NO: 1838. In some embodiments, the first polypeptide comprises the amino acid sequence of SEQ ID NO: 1839. In some embodiments, the first polypeptide comprises the amino acid sequence of SEQ ID NO: 1840. In some embodiments, the first polypeptide comprises the amino acid sequence of SEQ ID NO: 1859. In some embodiments, the first polypeptide comprises the amino acid sequence of SEQ ID NO: 1860. In some embodiments, the first polypeptide comprises the amino acid sequence of SEQ ID NO: 1861. In some embodiments, the first polypeptide comprises the amino acid sequence of SEQ ID NO: 1862. In some embodiments, the first polypeptide comprises the amino acid sequence of SEQ ID NO: 1879. In some embodiments, the first polypeptide comprises the amino acid sequence of SEQ ID NO: 1880. In some embodiments, the first polypeptide comprises the amino acid sequence of SEQ ID NO: 1881.

In some embodiments, the first polypeptide comprises the amino acid sequence of any one of SEQ ID NOs: 1596, 1597, 1615, 1626, 1818, 1822, 1825, or 1826. In some embodiments, the first polypeptide comprises the amino acid sequence of any one of SEQ ID NOs: 1596, 1597, 1615, or 1626. In some embodiments, the first polypeptide comprises the amino acid sequence of any one of SEQ ID NOs: 1626, 1818, 1822, 1825, or 1826.

In some embodiments, the polypeptide comprises the amino acid sequence of any one of SEQ ID NOs: 1587, 1592, 1615, 1626, 1818, 1822, 1825, or 1826. In some embodiments, the polypeptide comprises the amino acid sequence of any one of SEQ ID NOs: 1587, 1592, 1615, or 1626.

In some embodiments, the first polypeptide consists of the amino acid sequence of SEQ ID NO: 1587. In some embodiments, the first polypeptide consists of the amino acid sequence of SEQ ID NO: 1588. In some embodiments, the first polypeptide consists of the amino acid sequence of SEQ ID NO: 1589. In some embodiments, the first polypeptide consists of the amino acid sequence of SEQ ID NO: 1590. In some embodiments, the first polypeptide consists of the amino acid sequence of SEQ ID NO: 1591. In some embodiments, the first polypeptide consists of the amino acid sequence of SEQ ID NO: 1592. In some embodiments, the first polypeptide consists of the amino acid sequence of SEQ ID NO: 1593. In some embodiments, the first polypeptide consists of the amino acid sequence of SEQ ID NO: 1594. In some embodiments, the first polypeptide consists of the amino acid sequence of SEQ ID NO: 1595. In some embodiments, the first polypeptide consists of the amino acid sequence of SEQ ID NO: 1596. In some embodiments, the first polypeptide consists of the amino acid sequence of SEQ ID NO: 1597. In some embodiments, the first polypeptide consists of the amino acid sequence of SEQ ID NO: 1598. In some embodiments, the first polypeptide consists of the amino acid sequence of SEQ ID NO: 1599. In some embodiments, the first polypeptide consists of the amino acid sequence of SEQ ID NO: 1600. In some embodiments, the first polypeptide consists of the amino acid sequence of SEQ ID NO: 1601. In some embodiments, the first polypeptide consists of the amino acid sequence of SEQ ID NO: 1602. In some embodiments, the first polypeptide consists of the amino acid sequence of SEQ ID NO: 1603. In some embodiments, the first polypeptide consists of the amino acid sequence of SEQ ID NO: 1604. In some embodiments, the first polypeptide consists of the amino acid sequence of SEQ ID NO: 1605. In some embodiments, the first polypeptide consists of the amino acid sequence of SEQ ID NO: 1606. In some embodiments, the first polypeptide consists of the amino acid sequence of SEQ ID NO: 1607. In some embodiments, the first polypeptide consists of the amino acid sequence of SEQ ID NO: 1608. In some embodiments, the first polypeptide consists of the amino acid sequence of SEQ ID NO: 1609. In some embodiments, the first polypeptide consists of the amino acid sequence of SEQ ID NO: 1610. In some embodiments, the first polypeptide consists of the amino acid sequence of SEQ ID NO: 1611. In some embodiments, the first polypeptide consists of the amino acid sequence of SEQ ID NO: 1612. In some embodiments, the first polypeptide consists of the amino acid sequence of SEQ ID NO: 1613. In some embodiments, the first polypeptide consists of the amino acid sequence of SEQ ID NO: 1614. In some embodiments, the first polypeptide consists of the amino acid sequence of SEQ ID NO: 1615. In some embodiments, the first polypeptide consists of the amino acid sequence of SEQ ID NO: 1616. In some embodiments, the first polypeptide consists of the amino acid sequence of SEQ ID NO: 1617. In some embodiments, the first polypeptide consists of the amino acid sequence of SEQ ID NO: 1618. In some embodiments, the first polypeptide consists of the amino acid sequence of SEQ ID NO: 1619. In some embodiments, the first polypeptide consists of the amino acid sequence of SEQ ID NO: 1620. In some embodiments, the first polypeptide consists of the amino acid sequence of SEQ ID NO: 1621. In some embodiments, the first polypeptide consists of the amino acid sequence of SEQ ID NO: 1622. In some embodiments, the first polypeptide consists of the amino acid sequence of SEQ ID NO: 1623. In some embodiments, the first polypeptide consists of the amino acid sequence of SEQ ID NO: 1624. In some embodiments, the first polypeptide consists of the amino acid sequence of SEQ ID NO: 1625. In some embodiments, the first polypeptide consists of the amino acid sequence of SEQ ID NO: 1626. In some embodiments, the first polypeptide consists of the amino acid sequence of SEQ ID NO: 1627.

In some embodiments, the first polypeptide consists of the amino acid sequence of SEQ ID NO: 1747. In some embodiments, the first polypeptide consists of the amino acid sequence of SEQ ID NO: 1748. In some embodiments, the first polypeptide consists of the amino acid sequence of SEQ ID NO: 1749. In some embodiments, the first polypeptide consists of the amino acid sequence of SEQ ID NO: 1750. In some embodiments, the first polypeptide consists of the amino acid sequence of SEQ ID NO: 1751. In some embodiments, the first polypeptide consists of the amino acid sequence of SEQ ID NO: 1752. In some embodiments, the first polypeptide consists of the amino acid sequence of SEQ ID NO: 1753. In some embodiments, the first polypeptide consists of the amino acid sequence of SEQ ID NO: 1754. In some embodiments, the first polypeptide consists of the amino acid sequence of SEQ ID NO: 1755. In some embodiments, the first polypeptide consists of the amino acid sequence of SEQ ID NO: 1756. In some embodiments, the first polypeptide consists of the amino acid sequence of SEQ ID NO: 1757. In some embodiments, the first polypeptide consists of the amino acid sequence of SEQ ID NO: 1758. In some embodiments, the first polypeptide consists of the amino acid sequence of SEQ ID NO: 1759. In some embodiments, the first polypeptide consists of the amino acid sequence of SEQ ID NO: 1760. In some embodiments, the first polypeptide consists of the amino acid sequence of SEQ ID NO: 1761. In some embodiments, the first polypeptide consists of the amino acid sequence of SEQ ID NO: 1762. In some embodiments, the first polypeptide consists of the amino acid sequence of SEQ ID NO: 1763. In some embodiments, the first polypeptide consists of the amino acid sequence of SEQ ID NO: 1764. In some embodiments, the first polypeptide consists of the amino acid sequence of SEQ ID NO: 1765. In some embodiments, the first polypeptide consists of the amino acid sequence of SEQ ID NO: 1766. In some embodiments, the first polypeptide consists of the amino acid sequence of SEQ ID NO: 1767. In some embodiments, the first polypeptide consists of the amino acid sequence of SEQ ID NO: 1768. In some embodiments, the first polypeptide consists of the amino acid sequence of SEQ ID NO: 1769. In some embodiments, the first polypeptide consists of the amino acid sequence of SEQ ID NO: 1770. In some embodiments, the first polypeptide consists of the amino acid sequence of SEQ ID NO: 1771. In some embodiments, the first polypeptide consists of the amino acid sequence of SEQ ID NO: 1772. In some embodiments, the first polypeptide consists of the amino acid sequence of SEQ ID NO: 1773. In some embodiments, the first polypeptide consists of the amino acid sequence of SEQ ID NO: 1774. In some embodiments, the first polypeptide consists of the amino acid sequence of SEQ ID NO: 1775. In some embodiments, the first polypeptide consists of the amino acid sequence of SEQ ID NO: 1776. In some embodiments, the first polypeptide consists of the amino acid sequence of SEQ ID NO: 1777. In some embodiments, the first polypeptide consists of the amino acid sequence of SEQ ID NO: 1778. In some embodiments, the first polypeptide consists of the amino acid sequence of SEQ ID NO: 1779. In some embodiments, the first polypeptide consists of the amino acid sequence of SEQ ID NO: 1780. In some embodiments, the first polypeptide consists of the amino acid sequence of SEQ ID NO: 1781. In some embodiments, the first polypeptide consists of the amino acid sequence of SEQ ID NO: 1782. In some embodiments, the first polypeptide consists of the amino acid sequence of SEQ ID NO: 1783. In some embodiments, the first polypeptide consists of the amino acid sequence of SEQ ID NO: 1784. In some embodiments, the first polypeptide consists of the amino acid sequence of SEQ ID NO: 1785. In some embodiments, the first polypeptide consists of the amino acid sequence of SEQ ID NO: 1786. In some embodiments, the first polypeptide consists of the amino acid sequence of SEQ ID NO: 1787. In some embodiments, the first polypeptide consists of the amino acid sequence of SEQ ID NO: 1788. In some embodiments, the first polypeptide consists of the amino acid sequence of SEQ ID NO: 1789. In some embodiments, the first polypeptide consists of the amino acid sequence of SEQ ID NO: 1790. In some embodiments, the first polypeptide consists of the amino acid sequence of SEQ ID NO: 1791. In some embodiments, the first polypeptide consists of the amino acid sequence of SEQ ID NO: 1792. In some embodiments, the first polypeptide consists of the amino acid sequence of SEQ ID NO: 1793. In some embodiments, the first polypeptide consists of the amino acid sequence of SEQ ID NO: 1794. In some embodiments, the first polypeptide consists of the amino acid sequence of SEQ ID NO: 1795. In some embodiments, the first polypeptide consists of the amino acid sequence of SEQ ID NO: 1796. In some embodiments, the first polypeptide consists of the amino acid sequence of SEQ ID NO: 1797. In some embodiments, the first polypeptide consists of the amino acid sequence of SEQ ID NO: 1798. In some embodiments, the first polypeptide consists of the amino acid sequence of SEQ ID NO: 1799. In some embodiments, the first polypeptide consists of the amino acid sequence of SEQ ID NO: 1800. In some embodiments, the first polypeptide consists of the amino acid sequence of SEQ ID NO: 1801. In some embodiments, the first polypeptide consists of the amino acid sequence of SEQ ID NO: 1802. In some embodiments, the first polypeptide consists of the amino acid sequence of SEQ ID NO: 1803. In some embodiments, the first polypeptide consists of the amino acid sequence of SEQ ID NO: 1804. In some embodiments, the first polypeptide consists of the amino acid sequence of SEQ ID NO: 1805. In some embodiments, the first polypeptide consists of the amino acid sequence of SEQ ID NO: 1806. In some embodiments, the first polypeptide consists of the amino acid sequence of SEQ ID NO: 1807. In some embodiments, the first polypeptide consists of the amino acid sequence of SEQ ID NO: 1808. In some embodiments, the first polypeptide consists of the amino acid sequence of SEQ ID NO: 1809. In some embodiments, the first polypeptide consists of the amino acid sequence of SEQ ID NO: 1810. In some embodiments, the first polypeptide consists of the amino acid sequence of SEQ ID NO: 1811. In some embodiments, the first polypeptide consists of the amino acid sequence of SEQ ID NO: 1812. In some embodiments, the first polypeptide consists of the amino acid sequence of SEQ ID NO: 1813. In some embodiments, the first polypeptide consists of the amino acid sequence of SEQ ID NO: 1814. In some embodiments, the first polypeptide consists of the amino acid sequence of SEQ ID NO: 1815. In some embodiments, the first polypeptide consists of the amino acid sequence of SEQ ID NO: 1816. In some embodiments, the first polypeptide consists of the amino acid sequence of SEQ ID NO: 1817. In some embodiments, the first polypeptide consists of the amino acid sequence of SEQ ID NO: 1818. In some embodiments, the first polypeptide consists of the amino acid sequence of SEQ ID NO: 1819. In some embodiments, the first polypeptide consists of the amino acid sequence of SEQ ID NO: 1820. In some embodiments, the first polypeptide consists of the amino acid sequence of SEQ ID NO: 1821. In some embodiments, the first polypeptide consists of the amino acid sequence of SEQ ID NO: 1822. In some embodiments, the first polypeptide consists of the amino acid sequence of SEQ ID NO: 1823. In some embodiments, the first polypeptide consists of the amino acid sequence of SEQ ID NO: 1824. In some embodiments, the first polypeptide consists of the amino acid sequence of SEQ ID NO: 1825. In some embodiments, the first polypeptide consists of the amino acid sequence of SEQ ID NO: 1826. In some embodiments, the first polypeptide consists of the amino acid sequence of SEQ ID NO: 1827. In some embodiments, the first polypeptide consists of the amino acid sequence of SEQ ID NO: 1828. In some embodiments, the first polypeptide consists of the amino acid sequence of SEQ ID NO: 1829. In some embodiments, the first polypeptide consists of the amino acid sequence of SEQ ID NO: 1830. In some embodiments, the first polypeptide consists of the amino acid sequence of SEQ ID NO: 1831. In some embodiments, the first polypeptide consists of the amino acid sequence of SEQ ID NO: 1832. In some embodiments, the first polypeptide consists of the amino acid sequence of SEQ ID NO: 1833. In some embodiments, the first polypeptide consists of the amino acid sequence of SEQ ID NO: 1834. In some embodiments, the first polypeptide consists of the amino acid sequence of SEQ ID NO: 1835. In some embodiments, the first polypeptide consists of the amino acid sequence of SEQ ID NO: 1836. In some embodiments, the first polypeptide consists of the amino acid sequence of SEQ ID NO: 1837. In some embodiments, the first polypeptide consists of the amino acid sequence of SEQ ID NO: 1838. In some embodiments, the first polypeptide consists of the amino acid sequence of SEQ ID NO: 1839. In some embodiments, the first polypeptide consists of the amino acid sequence of SEQ ID NO: 1840. In some embodiments, the first polypeptide consists of the amino acid sequence of SEQ ID NO: 1859. In some embodiments, the first polypeptide consists of the amino acid sequence of SEQ ID NO: 1860. In some embodiments, the first polypeptide consists of the amino acid sequence of SEQ ID NO: 1861. In some embodiments, the first polypeptide consists of the amino acid sequence of SEQ ID NO: 1862.

In some embodiments, the first polypeptide consists of the amino acid sequence of any one of SEQ ID NOs: 1596, 1597, 1615, 1626, 1818, 1822, 1825, or 1826. In some embodiments, the first polypeptide consists of the amino acid sequence of any one of SEQ ID NOs: 1596, 1597, 1615, or 1626. In some embodiments, the first polypeptide consists of the amino acid sequence of any one of SEQ ID NOs: 1626, 1818, 1822, 1825, or 1826.

In some embodiments, the first polypeptide consists of the amino acid sequence of any one of SEQ ID NOs: 1587, 1592, 1615, 1626, 1818, 1822, 1825, or 1826. In some embodiments, the first polypeptide consists of the amino acid sequence of any one of SEQ ID NOs: 1587, 1592, 1615, or 1626.

In some embodiments, the second polypeptide comprises the amino acid sequence of SEQ ID NO: 1628. In some embodiments, the second polypeptide comprises the amino acid sequence of SEQ ID NO: 1629. In some embodiments, the second polypeptide comprises the amino acid sequence of SEQ ID NO: 1630. In some embodiments, the second polypeptide comprises the amino acid sequence of SEQ ID NO: 1631. In some embodiments, the second polypeptide comprises the amino acid sequence of SEQ ID NO: 1632. In some embodiments, the second polypeptide comprises the amino acid sequence of SEQ ID NO: 1633. In some embodiments, the second polypeptide comprises the amino acid sequence of SEQ ID NO: 1634. In some embodiments, the second polypeptide comprises the amino acid sequence of SEQ ID NO: 1635. In some embodiments, the second polypeptide comprises the amino acid sequence of SEQ ID NO: 1636. In some embodiments, the second polypeptide comprises the amino acid sequence of SEQ ID NO: 1637. In some embodiments, the second polypeptide comprises the amino acid sequence of SEQ ID NO: 1638. In some embodiments, the second polypeptide comprises the amino acid sequence of SEQ ID NO: 1639. In some embodiments, the second polypeptide comprises the amino acid sequence of SEQ ID NO: 1640. In some embodiments, the second polypeptide comprises the amino acid sequence of SEQ ID NO: 1641. In some embodiments, the second polypeptide comprises the amino acid sequence of SEQ ID NO: 1642. In some embodiments, the second polypeptide comprises the amino acid sequence of SEQ ID NO: 1643. In some embodiments, the second polypeptide comprises the amino acid sequence of SEQ ID NO: 1644. In some embodiments, the second polypeptide comprises the amino acid sequence of SEQ ID NO: 1645. In some embodiments, the second polypeptide comprises the amino acid sequence of SEQ ID NO: 1646. In some embodiments, the second polypeptide comprises the amino acid sequence of SEQ ID NO: 1647. In some embodiments, the second polypeptide comprises the amino acid sequence of SEQ ID NO: 1648. In some embodiments, the second polypeptide comprises the amino acid sequence of SEQ ID NO: 1649. In some embodiments, the second polypeptide comprises the amino acid sequence of SEQ ID NO: 1650. In some embodiments, the second polypeptide comprises the amino acid sequence of SEQ ID NO: 1651. In some embodiments, the second polypeptide comprises the amino acid sequence of SEQ ID NO: 1652. In some embodiments, the second polypeptide comprises the amino acid sequence of SEQ ID NO: 1653. In some embodiments, the second polypeptide comprises the amino acid sequence of SEQ ID NO: 1654. In some embodiments, the second polypeptide comprises the amino acid sequence of SEQ ID NO: 1655. In some embodiments, the second polypeptide comprises the amino acid sequence of SEQ ID NO: 1656. In some embodiments, the second polypeptide comprises the amino acid sequence of SEQ ID NO: 1657. In some embodiments, the second polypeptide comprises the amino acid sequence of SEQ ID NO: 1658. In some embodiments, the second polypeptide comprises the amino acid sequence of SEQ ID NO: 1659. In some embodiments, the second polypeptide comprises the amino acid sequence of SEQ ID NO: 1660. In some embodiments, the second polypeptide comprises the amino acid sequence of SEQ ID NO: 1661. In some embodiments, the second polypeptide comprises the amino acid sequence of SEQ ID NO: 1662. In some embodiments, the second polypeptide comprises the amino acid sequence of SEQ ID NO: 1663. In some embodiments, the second polypeptide comprises the amino acid sequence of SEQ ID NO: 1664. In some embodiments, the second polypeptide comprises the amino acid sequence of SEQ ID NO: 1665. In some embodiments, the second polypeptide comprises the amino acid sequence of SEQ ID NO: 1666. In some embodiments, the second polypeptide comprises the amino acid sequence of SEQ ID NO: 1667. In some embodiments, the second polypeptide comprises the amino acid sequence of SEQ ID NO: 1668. In some embodiments, the second polypeptide comprises the amino acid sequence of SEQ ID NO: 1669. In some embodiments, the second polypeptide comprises the amino acid sequence of SEQ ID NO: 1670. In some embodiments, the second polypeptide comprises the amino acid sequence of SEQ ID NO: 1671. In some embodiments, the second polypeptide comprises the amino acid sequence of SEQ ID NO: 1672. In some embodiments, the second polypeptide comprises the amino acid sequence of SEQ ID NO: 1673. In some embodiments, the second polypeptide comprises the amino acid sequence of SEQ ID NO: 1674. In some embodiments, the second polypeptide comprises the amino acid sequence of SEQ ID NO: 1675. In some embodiments, the second polypeptide comprises the amino acid sequence of SEQ ID NO: 1676. In some embodiments, the second polypeptide comprises the amino acid sequence of SEQ ID NO: 1677. In some embodiments, the second polypeptide comprises the amino acid sequence of SEQ ID NO: 1678. In some embodiments, the second polypeptide comprises the amino acid sequence of SEQ ID NO: 1679. In some embodiments, the second polypeptide comprises the amino acid sequence of SEQ ID NO: 1680. In some embodiments, the second polypeptide comprises the amino acid sequence of SEQ ID NO: 1681. In some embodiments, the second polypeptide comprises the amino acid sequence of SEQ ID NO: 1682. In some embodiments, the second polypeptide comprises the amino acid sequence of SEQ ID NO: 1683. In some embodiments, the second polypeptide comprises the amino acid sequence of SEQ ID NO: 1739. In some embodiments, the second polypeptide comprises the amino acid sequence of SEQ ID NO: 1740. In some embodiments, the second polypeptide comprises the amino acid sequence of SEQ ID NO: 1741. In some embodiments, the second polypeptide comprises the amino acid sequence of SEQ ID NO: 1742. In some embodiments, the second polypeptide comprises the amino acid sequence of SEQ ID NO: 1743. In some embodiments, the second polypeptide comprises the amino acid sequence of SEQ ID NO: 1744. In some embodiments, the second polypeptide comprises the amino acid sequence of SEQ ID NO: 1745. In some embodiments, the second polypeptide comprises the amino acid sequence of SEQ ID NO: 1746. In some embodiments, the second polypeptide comprises the amino acid sequence of SEQ ID NO: 1841. In some embodiments, the second polypeptide comprises the amino acid sequence of SEQ ID NO: 1842. In some embodiments, the second polypeptide comprises the amino acid sequence of SEQ ID NO: 1843. In some embodiments, the second polypeptide comprises the amino acid sequence of SEQ ID NO: 1844. In some embodiments, the second polypeptide comprises the amino acid sequence of SEQ ID NO: 1845. In some embodiments, the second polypeptide comprises the amino acid sequence of SEQ ID NO: 1846. In some embodiments, the second polypeptide comprises the amino acid sequence of SEQ ID NO: 1847. In some embodiments, the second polypeptide comprises the amino acid sequence of SEQ ID NO: 1848. In some embodiments, the second polypeptide comprises the amino acid sequence of SEQ ID NO: 1849. In some embodiments, the second polypeptide comprises the amino acid sequence of SEQ ID NO: 1850. In some embodiments, the second polypeptide comprises the amino acid sequence of SEQ ID NO: 1851. In some embodiments, the second polypeptide comprises the amino acid sequence of SEQ ID NO: 1852.

In some embodiments, the second polypeptide comprises the amino acid sequence of SEQ ID NO: 1628-1630. In some embodiments, the second polypeptide comprises the amino acid sequence of SEQ ID NO: 1628. In some embodiments, the second polypeptide comprises the amino acid sequence of SEQ ID NO: 1629. In some embodiments, the second polypeptide comprises the amino acid sequence of SEQ ID NO: 1630.

In some embodiments, the first polypeptide comprises at least 25 amino acids, wherein: the first polypeptide comprises 5-bromo-tryptophan at position 25; and the first polypeptide has at least 79% (e.g., at least 82%, at least 86%, at least 89%, at least 93%, at least 96%) sequence identity to the amino acid sequence of SEQ ID NO: 1587; and the second polypeptide comprises the amino acid sequence of SEQ ID NO: 1628-1630. In some embodiments, the first polypeptide comprises SEQ ID NO: 1587; and the second polypeptide comprises the amino acid sequence of SEQ ID NO: 1628-1630. In some embodiments, the second polypeptide comprises the amino acid sequence of SEQ ID NO: 1628. In some embodiments, the second polypeptide comprises the amino acid sequence of SEQ ID NO: 1629. In some embodiments, the second polypeptide comprises the amino acid sequence of SEQ ID NO: 1630.

In some embodiments, the first polypeptide comprises at least 28 amino acids, wherein: the first polypeptide comprises 2-aminoisobutyric acid at position 16 and lysine, serine, or aspartic acid at position 28; and the first polypeptide has at least 89% (e.g., at least 93%, at least 96%) sequence identity to the amino acid sequence of SEQ ID NO: 1596; and the second polypeptide comprises the amino acid sequence of SEQ ID NO: 1628-1630. In some embodiments, the first polypeptide comprises SEQ ID NO: 1596; and the second polypeptide comprises the amino acid sequence of SEQ ID NO: 1628-1630. In some embodiments, the second polypeptide comprises the amino acid sequence of SEQ ID NO: 1628. In some embodiments, the second polypeptide comprises the amino acid sequence of SEQ ID NO: 1629. In some embodiments, the second polypeptide comprises the amino acid sequence of SEQ ID NO: 1630.

In some embodiments, the first polypeptide comprises at least 28 amino acids, wherein: the first polypeptide comprises 2-aminoisobutyric acid at position 16, leucine at position 27, and lysine at position 28; and the first polypeptide has at least 89% (e.g., at least 93%, at least 96%) sequence identity to the amino acid sequence of SEQ ID NO: 1615; and the second polypeptide comprises the amino acid sequence of SEQ ID NO: 1628-1630. In some embodiments, the first polypeptide comprises SEQ ID NO: 1615; and the second polypeptide comprises the amino acid sequence of SEQ ID NO: 1628-1630. In some embodiments, the second polypeptide comprises the amino acid sequence of SEQ ID NO: 1628. In some embodiments, the second polypeptide comprises the amino acid sequence of SEQ ID NO: 1629. In some embodiments, the second polypeptide comprises the amino acid sequence of SEQ ID NO: 1630.

In some embodiments, the first polypeptide comprises at least 28 amino acids, wherein: the first polypeptide comprises 2-aminoisobutyric acid at position 16, aspartic acid at position 24, and lysine at position 28; and the first polypeptide has at least 89% (e.g., at least 93%, at least 96%) sequence identity to the amino acid sequence of SEQ ID NO: 1626; and the second polypeptide comprises the amino acid sequence of SEQ ID NO: 1628-1630. In some embodiments, the first polypeptide comprises SEQ ID NO: 1626; and the second polypeptide comprises the amino acid sequence of SEQ ID NO: 1628-1630. In some embodiments, the second polypeptide comprises the amino acid sequence of SEQ ID NO: 1628. In some embodiments, the second polypeptide comprises the amino acid sequence of SEQ ID NO: 1629. In some embodiments, the second polypeptide comprises the amino acid sequence of SEQ ID NO: 1630.

In some embodiments, the first polypeptide comprises at least 28 amino acids, wherein: the first polypeptide comprises d-serine at position 2 and 2-aminoisobutyric acid at position 16; and the first polypeptide has at least 89% (e.g., at least 93%, at least 96%) sequence identity to the amino acid sequence of SEQ ID NO: 1822; and the second polypeptide comprises the amino acid sequence of SEQ ID NO: 1628-1630. In some embodiments, the first polypeptide comprises SEQ ID NO: 1822; and the second polypeptide comprises the amino acid sequence of SEQ ID NO: 1628-1630. In some embodiments, the second polypeptide comprises the amino acid sequence of SEQ ID NO: 1628. In some embodiments, the second polypeptide comprises the amino acid sequence of SEQ ID NO: 1629. In some embodiments, the second polypeptide comprises the amino acid sequence of SEQ ID NO: 1630.

In some embodiments, the first polypeptide comprises the amino acid sequence of any one of SEQ ID NOs: 1587 1627, 1747-1840, 1859-1862, or 1879-1881; and the second polypeptide comprises the amino acid sequence of SEQ ID NO: 1628-1630. In some embodiments, the second polypeptide comprises the amino acid sequence of SEQ ID NO: 1628. In some embodiments, the second polypeptide comprises the amino acid sequence of SEQ ID NO: 1629. In some embodiments, the second polypeptide comprises the amino acid sequence of SEQ ID NO: 1630.

In some embodiments, the first polypeptide comprises the amino acid sequence of any one of SEQ ID NOs: 1587-1627; and the second polypeptide comprises the amino acid sequence of SEQ ID NO: 1628-1630. In some embodiments, the second polypeptide comprises the amino acid sequence of SEQ ID NO: 1628. In some embodiments, the second polypeptide comprises the amino acid sequence of SEQ ID NO: 1629. In some embodiments, the second polypeptide comprises the amino acid sequence of SEQ ID NO: 1630.

In some embodiments, the first polypeptide comprises the amino acid sequence of any one of SEQ ID NOs: 1596, 1597, 1615, 1626, 1818, 1822, 1825, or 1826; and the second polypeptide comprises the amino acid sequence of SEQ ID NO: 1628-1630. In some embodiments, the second polypeptide comprises the amino acid sequence of SEQ ID NO: 1628. In some embodiments, the second polypeptide comprises the amino acid sequence of SEQ ID NO: 1629. In some embodiments, the second polypeptide comprises the amino acid sequence of SEQ ID NO: 1630.

In some embodiments, the first polypeptide comprises the amino acid sequence of any one of SEQ ID NOs: 1587, 1592, 1596, 1615, or 1626; and the second polypeptide comprises the amino acid sequence of SEQ ID NO: 1628-1630. In some embodiments, the first polypeptide comprises the amino acid sequence of any one of SEQ ID NOs: 1587, 1592, 1615, or 1626. In some embodiments, the second polypeptide comprises the amino acid sequence of SEQ ID NO: 1628. In some embodiments, the second polypeptide comprises the amino acid sequence of SEQ ID NO: 1629. In some embodiments, the second polypeptide comprises the amino acid sequence of SEQ ID NO: 1630.

In some embodiments, the first polypeptide comprises the amino acid sequence of any one of SEQ ID NOs: 1587, 1592, 1615, 1626, 1818, 1822, 1825, or 1826; and the second polypeptide comprises the amino acid sequence of SEQ ID NO: 1628-1630. In some embodiments, the second polypeptide comprises the amino acid sequence of SEQ ID NO: 1628. In some embodiments, the second polypeptide comprises the amino acid sequence of SEQ ID NO: 1629. In some embodiments, the second polypeptide comprises the amino acid sequence of SEQ ID NO: 1630.

In some embodiments, the first polypeptide comprises the amino acid sequence of any one of SEQ ID NOs: 1626, 1818, 1822, 1825, or 1826; and the second polypeptide comprises the amino acid sequence of SEQ ID NO: 1628-1630. In some embodiments, the second polypeptide comprises the amino acid sequence of SEQ ID NO: 1628. In some embodiments, the second polypeptide comprises the amino acid sequence of SEQ ID NO: 1629. In some embodiments, the second polypeptide comprises the amino acid sequence of SEQ ID NO: 1630.

In some embodiments, a polypeptide or molecule of this disclosure comprises SEQ ID NOs: 1863 and 1864, as illustrated below:

```
HsQGT FTSDY SKYLD [Aib] KRAQ DFVD[5-BrW] LLKT a
                                                                                      |
                              GGGGS GGGGS PAPAP APAPA PASGG,
```

wherein the solid line between the lysine (K) residue and the glycine (G) residue represents an amide bond formed by condensation of the ε-amino group of the lysine residue and the C-terminal carboxylic acid group of the glycine residue. In some embodiments, the N-terminal glycine residue of the molecule is optionally derivatized to facilitate conjugation to another molecule. In some embodiments, the N-terminal glycine residue of the molecule is bromoacetylated to facilitate a thiol-bromoacetyl reaction with a thiol group (e.g., of a cysteine residue).

In some embodiments, a polypeptide or molecule of this disclosure comprises SEQ ID NOs: 1865 and 1866, as illustrated below:

```
HsQGT FTSDY SKYLD [Aib] KRAQ DFVDW LLKT
                                                                          |
                  GSPAP APAPA PAPAP APAPA PASGG,
```

wherein the solid line between the lysine (K) residue and the glycine (G) residue represents an amide bond formed by condensation of the ε-amino group of the lysine residue and the C-terminal carboxylic acid group of the glycine residue. In some embodiments, the N-terminal glycine residue of the molecule is optionally derivatized to facilitate conjugation to another molecule. In some embodiments, the N-terminal glycine residue of the molecule is bromoacetylated to facilitate a thiol-bromoacetyl reaction with a thiol group (e.g., of a cysteine residue).

In some embodiments, a polypeptide or molecule of this disclosure comprises SEQ ID NOs: 1867 and 1868, as illustrated below:

```
HsQGT FTSDY SKYLD [Aib] KRAQ DFVDW LLKT
                                                                          |
                  GGGGS GGGGS PAPAP APAPA PASGG,
```

wherein the solid line between the lysine (K) residue and the glycine (G) residue represents an amide bond formed by condensation of the ε-amino group of the lysine residue and the C-terminal carboxylic acid group of the glycine residue. In some embodiments, the N-terminal glycine residue of the molecule is optionally derivatized to facilitate conjugation to another molecule. In some embodiments, the N-terminal glycine residue of the molecule is bromoacetylated to facilitate a thiol-bromoacetyl reaction with a thiol group (e.g., of a cysteine residue).

In some embodiments, a polypeptide or molecule of this disclosure comprises SEQ ID NOs: 1869 and 1870, as illustrated below:

```
HsQGT FTSDY SKYLD [Aib] KRAQ EFVEW LLKS A
                                                                          |
                  GGGGS GGGGS PAPAP APAPA PASGG,
```

wherein the solid line between the lysine (K) residue and the glycine (G) residue represents an amide bond formed by condensation of the ε-amino group of the lysine residue and the C-terminal carboxylic acid group of the glycine residue. In some embodiments, the N-terminal glycine residue of the molecule is optionally derivatized to facilitate conjugation to another molecule. In some embodiments, the N-terminal glycine residue of the molecule is bromoacetylated to facilitate a thiol-bromoacetyl reaction with a thiol group (e.g., of a cysteine residue).

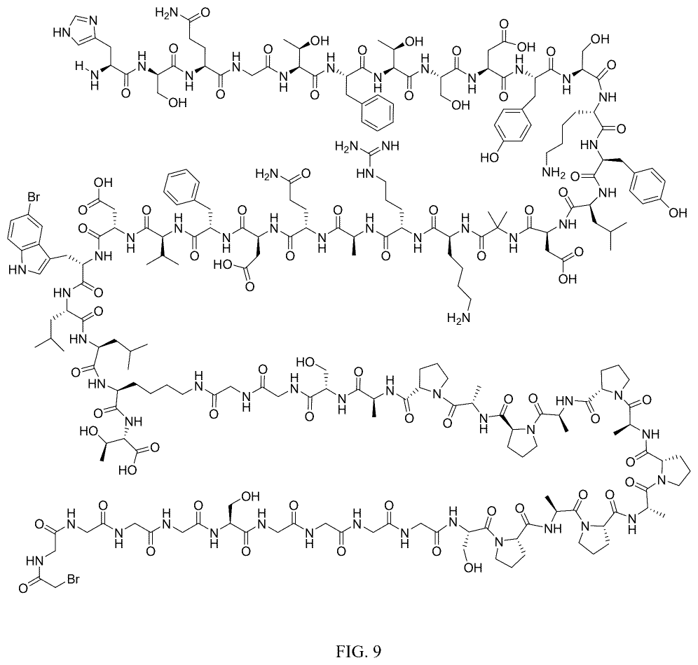

In some embodiments, a molecule or polypeptide of this disclosure comprises the structure depicted in FIG. 9.

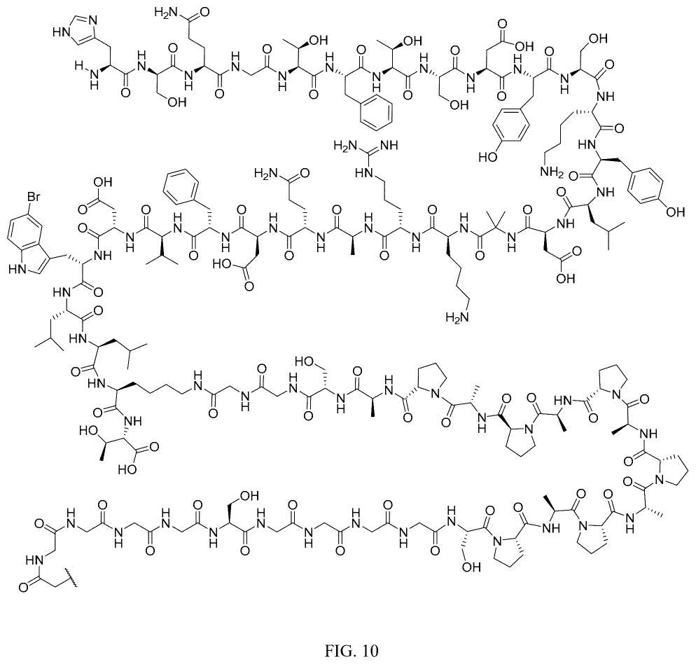

In some embodiments, a molecule or polypeptide of this disclosure comprises the structure depicted in FIG. 10.

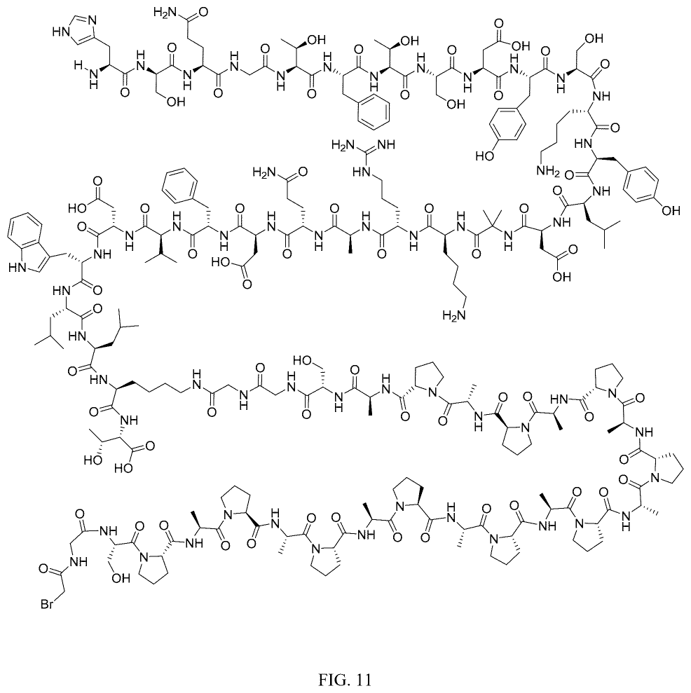

In some embodiments, a molecule or polypeptide of this disclosure comprises the structure depicted in FIG. 11.

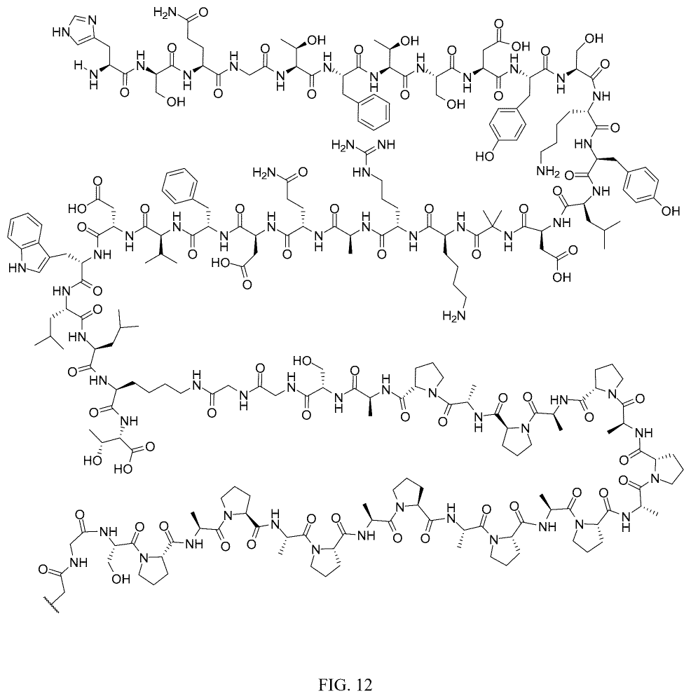

In some embodiments, a molecule or polypeptide of this disclosure comprises the structure depicted in FIG. 12.

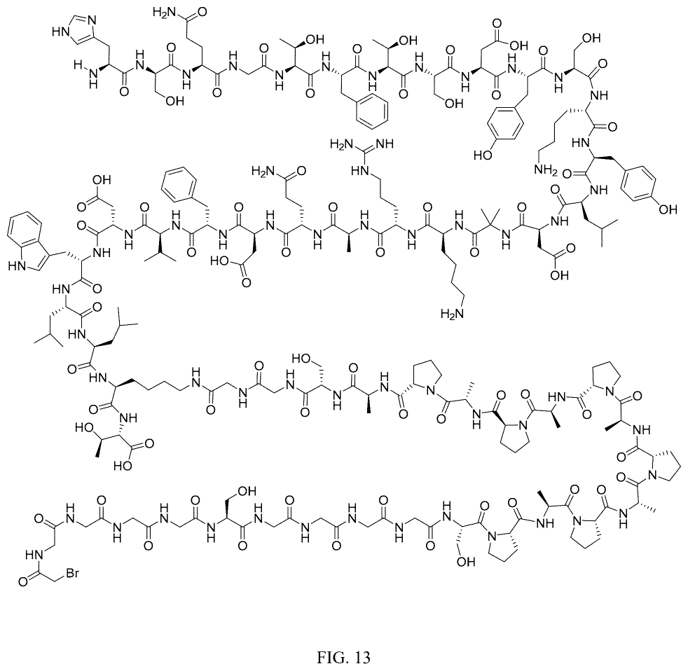

In some embodiments, a molecule or polypeptide of this disclosure comprises the structure depicted in FIG. 13.

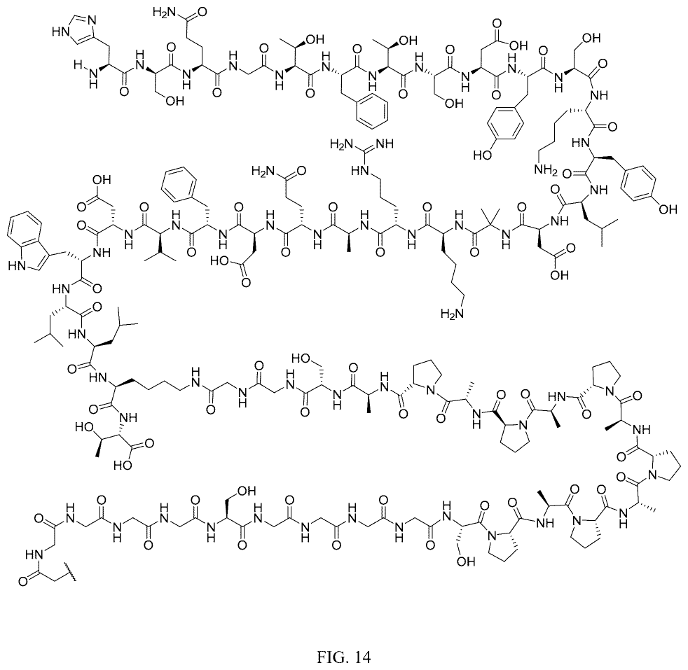

In some embodiments, a molecule or polypeptide of this disclosure comprises the structure depicted in FIG. 14.

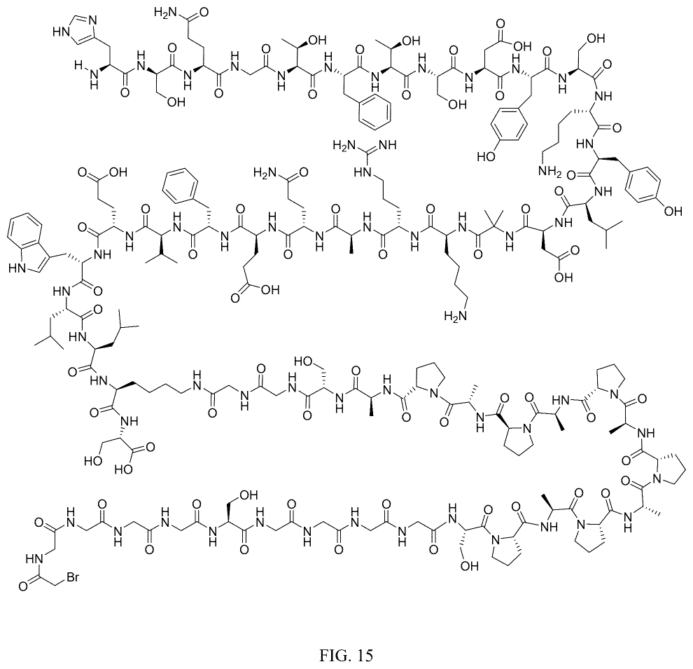

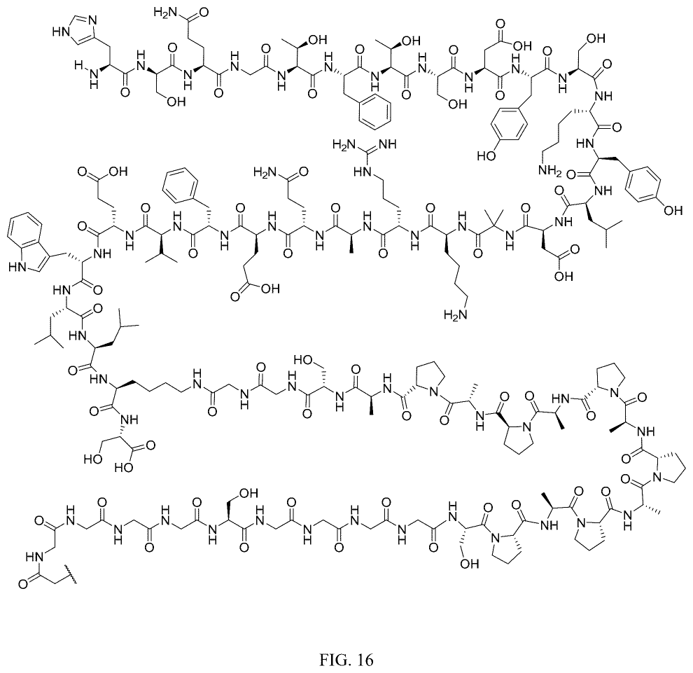

In some embodiments, a molecule or polypeptide of this disclosure comprises the structure depicted in FIG. 15.

In some embodiments, a molecule or polypeptide of this disclosure comprises the structure depicted in FIG. 16.

Also provided herein is a molecule comprising a first polypeptide that agonizes a GCGR, wherein the first polypeptide is selected from those described herein (e.g., a polypeptide comprising the amino acid sequence of any one of SEQ ID NOs: 1587-1627, 1747-1840, 1859-1862, or 1879-1881; SEQ ID NOs: 1587-1627 or 1747; SEQ ID NOs: 1587-1627; SEQ ID NOs: 1748-1840, 1859-1862, or 1879-1881); and a second polypeptide comprising the amino acid sequence of any one of SEQ ID NOs: 1740-1746, 1841-1849, or 1852, wherein the C-terminal amino acid residue of the first polypeptide is covalently linked to the N-terminal amino acid residue of the second polypeptide. In some embodiments, the C-terminal amino acid residue of the second polypeptide is a lysine residue. In some embodiments, the C-terminal amino acid residue of the second polypeptide is modified for conjugation to a cysteine residue (e.g., bromoacetylated). In some embodiments, the C-terminal amino acid residue of the second polypeptide is a bromoacetylated lysine residue.

In some embodiments, the molecule is a branched polypeptide. In some embodiments, the molecule is a branched peptide.

In some embodiments, the molecule has a hGCGR EC₅₀ of less than or equal to 1 nM.

In some embodiments, the molecule has a hGCGR EC₅₀ of less than or equal to 500 pM (e.g., less than or equal to 475 pM, less than or equal to 450 pM, less than or equal to 425 pM, less than or equal to 400 pM, less than or equal to 375 pM, less than or equal to 350 pM, less than or equal to 325 pM, less than or equal to 300 pM, less than or equal to 275 pM, less than or equal to 250 pM, less than or equal to 225 pM, less than or equal to 200 pM, less than or equal to 175 pM, less than or equal to 150 pM, less than or equal to 125 pM, less than or equal to 100 pM). In some embodiments, the hGCGR EC₅₀ is measured in a functional assay described in “EXAMPLE 1: GCG Receptor Agonist Activity.”

In some embodiments, the molecule has a hGCGR EC₅₀ of 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 155 pM, 200 pM, 205 pM, 210 pM, 215 pM, 220 pM, 225 pM, 230 pM, 235 pM, 240 pM, 245 pM, 250 pM, 255 pM, 260 pM, 265 pM, 270 pM, 275 pM, 280 pM, 285 pM, 290 pM, 295 pM, 300 pM, 305 pM, 310 pM, 315 pM, 320 pM, 325 pM, 330 pM, 335 pM, 340 pM, 345 pM, 350 pM, 355 pM, 360 pM, 365 pM, 370 pM, 375 pM, 380 pM, 385 pM, 390 pM, 395 pM, 400 pM, 405 pM, 410 pM, 415 pM, 420 pM, 425 pM, 430 pM, 435 pM, 440 pM, 445 pM, 450 pM, 455 pM, 460 pM, 465 pM, 470 pM, 475 pM, 480 pM, 485 pM, 490 pM, 495 pM, or 500 pM. In some embodiments, the hGCGR EC₅₀ is measured in a functional assay described in “EXAMPLE 1: GCG Receptor Agonist Activity.”

In some embodiments, the molecule has a human glucagon-like peptide-1 receptor (“hGLP-1R”) EC₅₀:hGCGR EC₅₀ ratio of at least 30:1. In some embodiments, the molecule has a hGLP-1R EC₅₀:hGCGR EC₅₀ ratio of at least 40:1. In some embodiments, the molecule has a hGLP-1R EC₅₀:hGCGR EC₅₀ ratio of at least 50:1. In some embodiments, the molecule has a hGLP-1R EC₅₀:hGCGR EC₅₀ ratio of at least 60:1. In some embodiments, the molecule has a hGLP-1R EC₅₀:hGCGR EC₅₀ ratio of at least 70:1. In some embodiments, the molecule has a hGLP-1R EC₅₀:hGCGR EC₅₀ ratio of at least 80:1. In some embodiments, the molecule has a hGLP-1R EC₅₀:hGCGR EC₅₀ ratio of at least 90:1. In some embodiments, the molecule has a hGLP-1R EC₅₀:hGCGR EC₅₀ ratio of at least 100:1. In some embodiments, the molecule has a hGLP-1R EC₅₀:hGCGR EC₅₀ ratio of at least 150:1. In some embodiments, the molecule has a hGLP-1R EC₅₀:hGCGR EC₅₀ ratio of at least 200:1. In some embodiments, the molecule has a hGLP-1R EC₅₀:hGCGR EC₅₀ ratio of at least 250:1. In some embodiments, the molecule has a hGLP-1R EC₅₀:hGCGR EC₅₀ ratio of at least 300:1. In some embodiments, the molecule has a hGLP-1R EC₅₀:hGCGR EC₅₀ ratio of at least 350:1. In some embodiments, the molecule has a hGLP-1R EC₅₀:hGCGR EC₅₀ ratio of at least 400:1. In some embodiments, the molecule has a hGLP-1R EC₅₀:hGCGR EC₅₀ ratio of at least 450:1. In some embodiments, the molecule has a hGLP-1R EC₅₀:hGCGR EC₅₀ ratio of at least 500:1. In some embodiments, the molecule has a hGLP-1R EC₅₀:hGCGR EC₅₀ ratio of at least 550:1. In some embodiments, the molecule has a hGLP-1R EC₅₀:hGCGR EC₅₀ ratio of at least 650:1. In some embodiments, the molecule has a hGLP-1R EC₅₀:hGCGR EC₅₀ ratio of at least 700:1. In some embodiments, the molecule has a hGLP-1R EC₅₀:hGCGR EC₅₀ ratio of at least 750:1. In some embodiments, the molecule has a hGLP-1R EC₅₀:hGCGR EC₅₀ ratio of at least 800:1. In some embodiments, the molecule has a hGLP-1R EC₅₀:hGCGR EC₅₀ ratio of at least 850:1. In some embodiments, the molecule has a hGLP-1R EC₅₀:hGCGR EC₅₀ ratio of at least 900:1. In some embodiments, the molecule has a hGLP-1R EC₅₀:hGCGR EC₅₀ ratio of at least 950:1. In some embodiments, the molecule has a hGLP-1R EC₅₀:hGCGR EC₅₀ ratio of at least 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

In some embodiments, the molecule has a hGLP-1R EC₅₀:hGCGR EC₅₀ ratio of 30:1, 40:1, 45:1, 50:1, 55:1, 60:1, 65:1, 70:1, 75:1, 80:1, 85:1, 90:1, 95:1, 100:1, 125:1, 150:1, 175:1, 200:1, 225:1, 250:1, 275:1, 300:1, 325:1, 350:1, 375:1, 400:1, 425:1, 450:1, 475:1, 500:1, 525:1, 550:1, 575:1, 600:1, 625:1, 650:1, 675:1, 700:1, 725:1, 750:1, 775:1, 800:1, 825:1, 850:1, 875:1, 900:1, 925:1, 950:1, 975:1, or 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

Peptides described herein may be prepared by processes well-known in the art, e.g., peptide purification as described in Eng et al., J. Biol. Chem., 265:20259-62 (1990); standard solid-phase peptide synthesis techniques as described in Raufman et al., J. Biol. Chem., 267:21432-37 (1992) or liquid-phase peptide techniques as described in Sharma et al., Chem. Rev., 122(16):13516-13546 (2022); recombinant DNA techniques as described in Sambrook et al., Molecular Cloning: A Laboratory Manual, 2d Ed., Cold Spring Harbor (1989); and the like. Additionally, peptides provided herein can be chemically derivatized at one or more amino acid residues (e.g., N-terminal acetylation or C-terminal amidation) by known organic chemistry techniques.

Glucagon Receptor Agonist Conjugates with Half-Life Extending Domains

Provided herein is a molecule that comprises a polypeptide that agonizes a glucagon receptor (“GCGR”), wherein: the polypeptide is selected from the polypeptides described herein; and a half-life extending domain (e.g., an Fc-containing polypeptide). Such molecules can possess one or more desirable properties in addition to GCGR agonism, such as, e.g., an extended serum half-life.

Conjugation of a half-life extending domain to a polypeptide, either directly or via a linker moiety, can enable altered pharmacodynamics and pharmacokinetics relative to the unmodified polypeptide. For example, in some embodiments, a half-life extending domain can extend elimination half-time relative to the unmodified polypeptide. In addition, in some embodiments, a half-life extending domain can alter one or more pharmacodynamic properties of the polypeptide, such as, e.g., tissue distribution, penetration, or diffusion.

In some embodiments, the half-life extending domain is a molecule that specifically binds to a circulating plasma protein.

In some embodiments, the half-life extending domain is a molecule that specifically binds to an albumin. In some embodiments, the half-life extending domain is a polypeptide (e.g., an antibody, antibody fragment, or peptide) that specifically binds to an albumin.

In some embodiments, the half-life extending domain is a molecule that specifically binds to human serum albumin (HSA). In some embodiments, the half-life extending domain is a polypeptide (e.g., an antibody, antibody fragment, or peptide) that specifically binds to HSA.

In some embodiments, the half-life extending domain is HSA or a variant thereof.

In some embodiments, the half-life extending domain is a molecule that specifically binds to the neonatal Fc receptor (FcRn). In some embodiments, the half-life extending domain is a polypeptide (e.g., an antibody, antibody fragment, or peptide) that specifically binds to FcRn.

In some embodiments, the half-life extending domain is an Fc domain. In some embodiments, the half-life extending domain is an Fc domain described in Table S1 below. In some embodiments, the half-life extending domain comprises the amino acid sequence of SEQ ID NO: 1858.

In some embodiments, the half-life extending domain is an Fc-containing polypeptide. In some embodiments, the half-life extending domain is an antibody. In some embodiments, the half-life extending domain is an antibody fragment. In some embodiments, the half-life extending domain is an scFv.

In some embodiments, the half-life extending domain is a transferrin.

In some embodiments, the polypeptide comprises at least 25 amino acids, wherein: the polypeptide comprises 5-bromo-tryptophan at position 25; and the polypeptide has at least 79% (e.g., at least 82%, at least 86%, at least 89%, at least 93%, at least 96%) sequence identity to the amino acid sequence of SEQ ID NO: 1587. In some embodiments, the polypeptide comprises SEQ ID NO: 1587. In some embodiments, the polypeptide consists of SEQ ID NO: 1587.

In some embodiments, the polypeptide comprises at least 28 amino acids, wherein: the polypeptide comprises 2-aminoisobutyric acid at position 16 and lysine, serine, or aspartic acid at position 28; and the polypeptide has at least 89% (e.g., at least 93%, at least 96%) sequence identity to the amino acid sequence of SEQ ID NO: 1596. In some embodiments, the polypeptide comprises SEQ ID NO: 1596. In some embodiments, the polypeptide consists of SEQ ID NO: 1596.

In some embodiments, the polypeptide comprises at least 28 amino acids, wherein: the polypeptide comprises 2-aminoisobutyric acid at position 16, leucine at position 27, and lysine at position 28; and the polypeptide has at least 89% (e.g., at least 93%, at least 96%) sequence identity to the amino acid sequence of SEQ ID NO: 1615. In some embodiments, the polypeptide comprises SEQ ID NO: 1615. In some embodiments, the polypeptide consists of SEQ ID NO: 1615.

In some embodiments, the polypeptide comprises at least 28 amino acids, wherein: the polypeptide comprises 2-aminoisobutyric acid at position 16, aspartic acid at position 24, and lysine at position 28; and the polypeptide has at least 89% (e.g., at least 93%, at least 96%) sequence identity to the amino acid sequence of SEQ ID NO: 1626. In some embodiments, the polypeptide comprises SEQ ID NO: 1626. In some embodiments, the polypeptide consists of SEQ ID NO: 1626.

In some embodiments, the polypeptide comprises at least 28 amino acids, wherein: the polypeptide comprises d-serine at position 2 and 2-aminoisobutyric acid at position 16; and the polypeptide has at least 89% (e.g., at least 93%, at least 96%) sequence identity to the amino acid sequence of SEQ ID NO: 1822. In some embodiments, the polypeptide comprises SEQ ID NO: 1822. In some embodiments, the polypeptide consists of SEQ ID NO: 1822.

In some embodiments, the polypeptide comprises an amino acid sequence with between three and nine modifications relative to SEQ ID NO: 1576, wherein the modifications are selected from:
tyrosine and phenylalanine at position 1;
d-serine, 2-aminoisobutyric acid, and d-threonine at position 2;
glutamic acid at position 3;
histidine at position 7;
tryptophan at position 10;
glutamic acid at position 15;
2-aminoisobutyric acid, glutamine, homophenylalanine, and glutamic acid at position 16;
lysine, citrulline, glutamine, and alanine at position 17;
2-naphthylalanine, L-4,4′-biphenylalanine, alanine, citrulline, and lysine at position 18;
4-chloro-L-phenylalanine, alanine, d-glutamine, homoserine, histidine, arginine, and glutamic acid at position 20;
glutamic acid, citrulline, and d-aspartic acid at position 21;
tryptophan and β-cyclohexyl-L-alanine at position 22;
aspartic acid, lysine, alanine, 2-aminoisobutyric acid, glycine, histidine, asparagine, threonine, d-glutamine, glutamic acid, arginine, phenylalanine, leucine, serine, tyrosine, valine, isoleucine, homoserine, and 2,3-diaminopropionic acid at position 24;
5-bromo-L-tryptophan, tyrosine, L-beta-homotryptophan, 5-methoxy-L-tryptophan, 5-methyl-L-tryptophan, 6-bromo-L-tryptophan, 6-chloro-L-tryptophan, 6-methyl-L-tryptophan, and 7-bromo-L-tryptophan at position 25;
leucine, glutamic acid, and L-α-aminoadipic acid at position 27;
lysine, aspartic acid, serine, 6-azido-L-lysine, glutamic acid, and alanine at position 28;
glutamic acid, serine, aspartic acid, and alanine at position 29;
an additional amino acid at position 30, wherein the additional amino acid is lysine; and
an additional amino acid at position 31, wherein the additional amino acid is lysine.

In some embodiments, the modifications comprise a d-serine at position 2, a 2-aminoisobutyric acid at position 16, and between one and seven other modifications at position 1, 3, 7, 10, 15, 17, 18, 20, 21, 22, 24, 25, 26, 27, 28, 29, 30, or 31 (e.g., at position 17, 21, 24, 25, 27, 28, or 29) as described above. In some embodiments, the modifications are selected from:
lysine at position 17;
glutamic acid at position 21;
lysine, alanine, and glutamic acid at position 24;
5-bromo-L-tryptophan at position 25;
leucine and glutamic acid at position 27;
lysine, aspartic acid, and glutamic acid at position 28; and
glutamic acid and serine at position 29.

In some embodiments, the polypeptide comprises the amino acid sequence of any one of SEQ ID NOs: 1587-1627, 1747-1840, 1859-1862, or 1879-1881. In some embodiments, the polypeptide comprises the amino acid sequence of any one of SEQ ID NOs: 1587-1627 or 1747. In some embodiments, the polypeptide comprises the amino acid sequence of any one of SEQ ID NOs: 1747-1840, 1859-1862, or 1879-1881. In some embodiments, the polypeptide consists of the amino acid sequence of any one of SEQ ID NOs: 1587-1627, 1747-1840, 1859-1862, or 1879-1881. In some embodiments, the polypeptide consists of the amino acid sequence of any one of SEQ ID NOs: 1587-1627 or 1747. In some embodiments, the polypeptide consists of the amino acid sequence of any one of SEQ ID NOs: 1747-1840, 1859-1862, or 1879-1881.

In some embodiments, the polypeptide comprises the amino acid sequence of any one of SEQ ID NOs: 1587-1627. In some embodiments, the polypeptide consists of the amino acid sequence of any one of SEQ ID NOs: 1587-1627.

In some embodiments, the polypeptide comprises the amino acid sequence of any one of SEQ ID NOs: 1596, 1597, 1615, 1626, 1818, 1822, 1825, or 1826. In some embodiments, the polypeptide comprises the amino acid sequence of any one of SEQ ID NOs: 1587, 1592, 1596, 1615, or 1626. In some embodiments, the polypeptide comprises the amino acid sequence of any one of SEQ ID NOs: 1626, 1818, 1822, 1825, or 1826.

In some embodiments, the polypeptide comprises the amino acid sequence of SEQ ID NO: 1587. In some embodiments, the polypeptide comprises the amino acid sequence of SEQ ID NO: 1592. In some embodiments, the polypeptide comprises the amino acid sequence of SEQ ID NO: 1596. In some embodiments, the polypeptide comprises the amino acid sequence of SEQ ID NO: 1615. In some embodiments, the polypeptide comprises the amino acid sequence of SEQ ID NO: 1626.

In some embodiments, the polypeptide comprises the amino acid sequence of any one of SEQ ID NOs: 1588 or 1596-1611. In some embodiments, the polypeptide comprises the amino acid sequence of SEQ ID NO: 1588. In some embodiments, the polypeptide comprises the amino acid sequence of SEQ ID NO: 1596. In some embodiments, the polypeptide comprises the amino acid sequence of SEQ ID NO: 1597. In some embodiments, the polypeptide comprises the amino acid sequence of SEQ ID NO: 1598. In some embodiments, the polypeptide comprises the amino acid sequence of SEQ ID NO: 1599. In some embodiments, the polypeptide comprises the amino acid sequence of SEQ ID NO: 1600. In some embodiments, the polypeptide comprises the amino acid sequence of SEQ ID NO: 1601. In some embodiments, the polypeptide comprises the amino acid sequence of SEQ ID NO: 1602. In some embodiments, the polypeptide comprises the amino acid sequence of SEQ ID NO: 1603. In some embodiments, the polypeptide comprises the amino acid sequence of SEQ ID NO: 1604. In some embodiments, the polypeptide comprises the amino acid sequence of SEQ ID NO: 1605. In some embodiments, the polypeptide comprises the amino acid sequence of SEQ ID NO: 1606. In some embodiments, the polypeptide comprises the amino acid sequence of SEQ ID NO: 1607. In some embodiments, the polypeptide comprises the amino acid sequence of SEQ ID NO: 1608. In some embodiments, the polypeptide comprises the amino acid sequence of SEQ ID NO: 1609. In some embodiments, the polypeptide comprises the amino acid sequence of SEQ ID NO: 1610. In some embodiments, the polypeptide comprises the amino acid sequence of SEQ ID NO: 1611.

In some embodiments, the half-life extending domain (e.g., the Fc-containing polypeptide) is conjugated, i.e., covalently bound, directly to an amino acid residue of the polypeptide agonist, or optionally, to a peptidyl or non-peptidyl linker moiety (including, but not limited to, aromatic or aryl linkers) that is covalently bound to an amino acid residue of the polypeptide agonist. For example, in some embodiments, an β-amino group of a lysine residue of the polypeptide is covalently linked to a C-terminus of a linker polypeptide; and an N-terminus of the linker polypeptide is conjugated to a cysteine residue of a half-life extending domain (e.g., an Fc-containing polypeptide). Illustratively, in some embodiments, a lysine residue of the polypeptide is covalently linked to a C-terminus of a linker polypeptide via an amide bond, e.g., an amide bond formed by condensation of an ε-amino group of a lysine residue and a carboxyl group of a C-terminus of a polypeptide linker. Additionally, in some embodiments, the N-terminus of the linker polypeptide is derivatized. For example, in some embodiments, the N-terminus of the linker polypeptide is acetylated, such as when a thioether linkage connects an acetylated N-terminus of the linker polypeptide and the cysteine residue of the half-life extending domain (e.g., the Fc-containing polypeptide), wherein the thioether linkage comprises a sulfur atom of the cysteine residue.

Alternatively, in some embodiments, the C-terminal amino acid residue of the polypeptide is covalently linked to the N-terminal amino acid residue of the linker polypeptide. In some embodiments, the C-terminus of the linker polypeptide is derivatized. In some embodiments, the C-terminal amino acid residue of the linker polypeptide is modified for coupling to a cysteine residue (e.g., is bromoacetylated). Illustratively, in some embodiments, the C-terminus of the linker polypeptide is acetylated, such as when a thioether linkage connects an acetylated C-terminus of the linker polypeptide and the cysteine residue of the half-life extending domain (e.g., the Fc-containing polypeptide), wherein the thioether linkage comprises a sulfur atom of the cysteine residue.

In some embodiments, the linker polypeptide is a linear polypeptide.

In some embodiments, the linker polypeptide comprises the amino acid sequence of any one of SEQ ID NOs: 1628-1683, 1739-1746, or 1841-1852. In some embodiments, the linker polypeptide comprises the amino acid sequence of any one of SEQ ID NOs: 1628-1683, 1739, 1850, or 1851. In some embodiments, the linker polypeptide comprises the amino acid sequence of any one of SEQ ID NOs: 1740-1746, 1841-1849, or 1852.

In some embodiments, the linker polypeptide comprises the amino acid sequence of any one of SEQ ID NOs: 1628-1683. In some embodiments, the linker polypeptide comprises the amino acid sequence of any one of SEQ ID NOs: 1628-1630. In some embodiments, the linker polypeptide comprises the amino acid sequence of SEQ ID NO: 1628. In some embodiments, the linker polypeptide comprises the amino acid sequence of SEQ ID NO: 1629. In some embodiments, the linker polypeptide comprises the amino acid sequence of SEQ ID NO: 1630.

The presence of any linker moiety is optional. Non-limiting example linker moieties suitable for use in GCGR agonist conjugates with half-life extending domains (e.g., Fc-containing polypeptides) are described in more detail below.

In some embodiments, the half-life extending domain is an antigen-binding protein.

In some embodiments, the half-life extending domain is an antibody fragment.

In some embodiments, the half-life extending domain is an antibody. In some embodiments, the half-life extending domain is an isotype antibody such as 655-351, which is described in Table S1 below. In some embodiments, the half-life extending domain comprises a a heavy chain, wherein the heavy chain comprises an amino acid sequence selected from SEQ ID NOs: 1853-1855. In some embodiments, the heavy chain comprises the amino acid sequence of SEQ ID NO: 1853. In some embodiments, the heavy chain comprises the amino acid sequence of SEQ ID NO: 1854. In some embodiments, the heavy chain comprises the amino acid sequence of SEQ ID NO: 1855. In some embodiments, the half-life extending domain comprises a light chain, wherein the light chain comprises an amino acid sequence of SEQ ID NO: 1856 or SEQ ID NO: 1857.

In some embodiments, the half-life extending domain comprises a light chain and a heavy chain, wherein the light chain and heavy chain comprise the amino acid sequences of SEQ ID NO: 1856 and SEQ ID NO: 1853, respectively.

In some embodiments, the half-life extending domain comprises a light chain and a heavy chain, wherein the light chain and heavy chain comprise the amino acid sequences of SEQ ID NO: 1856 and SEQ ID NO: 1854, respectively.

In some embodiments, the half-life extending domain comprises a light chain and a heavy chain, wherein the light chain and heavy chain comprise the amino acid sequences of SEQ ID NO: 1857 and SEQ ID NO: 1855, respectively.

In some embodiments, the antibody is a monoclonal antibody, a recombinant antibody, a human antibody, a humanized antibody, or a chimeric antibody. In some embodiments, the antibody is a monoclonal antibody. In some embodiments, the antibody is a recombinant antibody. In some embodiments, the antibody is a human antibody. In some embodiments, the antibody is a humanized antibody. In some embodiments, the antibody is a chimeric antibody.

In some embodiments, the antibody is of the IgG1-, IgG2- IgG3-, or IgG4-type. In some embodiments, the antibody is of the IgG1-, IgG2-, or IgG4-subclass. In some embodiments, the antibody is of the IgG1- or IgG2-subclass.

In some embodiments, the antibody is of the IgG1-type. In some embodiments, the antibody is of the IgG2-type. In some embodiments, the antibody is of the IgG3-type. In some embodiments, the antibody is of the IgG4-type.

In some embodiments, the half-life extending domain is an antibody that specifically binds to 2,4-dinitrophenol (“DNP”). Conjugates with aDNP antibodies are described in more detail below.

In some embodiments, the half-life extending domain is an antibody that specifically binds to a glucose-dependent insulinotropic polypeptide receptor (“GIPR”). In some embodiments, the antibody specifically binds to human GIPR. In some embodiments, the antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise the amino acid sequences of SEQ ID NO: 702, SEQ ID NO: 859, SEQ ID NO: 1016, SEQ ID NO: 1173, SEQ ID NO: 1330, and SEQ ID NO: 1487, respectively. In some embodiments, the antibody comprises a light chain variable region comprising the amino acid sequence of SEQ ID NO: 74 and a heavy chain variable region comprising the amino acid sequence of SEQ ID NO: 231. In some embodiments, the antibody comprises a light chain comprising the amino acid sequence of SEQ ID NO: 388 and a heavy chain comprising the amino acid sequence of SEQ ID NO: 1571. Conjugates with aGIPR antibodies are described in more detail below.

In some embodiments, the molecule has a hGCGR EC₅₀ of less than or equal to 1 nM.

In some embodiments, the molecule has a hGCGR EC₅₀ of less than or equal to 500 pM (e.g., less than or equal to 475 pM, less than or equal to 450 pM, less than or equal to 425 pM, less than or equal to 400 pM, less than or equal to 375 pM, less than or equal to 350 pM, less than or equal to 325 pM, less than or equal to 300 pM, less than or equal to 275 pM, less than or equal to 250 pM, less than or equal to 225 pM, less than or equal to 200 pM, less than or equal to 175 pM, less than or equal to 150 pM, less than or equal to 125 pM, less than or equal to 100 pM). In some embodiments, the hGCGR EC₅₀ is measured in a functional assay described in “EXAMPLE 1: GCG Receptor Agonist Activity.”

In some embodiments, the molecule has a hGCGR EC₅₀ of 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 155 pM, 200 pM, 205 pM, 210 pM, 215 pM, 220 pM, 225 pM, 230 pM, 235 pM, 240 pM, 245 pM, 250 pM, 255 pM, 260 pM, 265 pM, 270 pM, 275 pM, 280 pM, 285 pM, 290 pM, 295 pM, 300 pM, 305 pM, 310 pM, 315 pM, 320 pM, 325 pM, 330 pM, 335 pM, 340 pM, 345 pM, 350 pM, 355 pM, 360 pM, 365 pM, 370 pM, 375 pM, 380 pM, 385 pM, 390 pM, 395 pM, 400 pM, 405 pM, 410 pM, 415 pM, 420 pM, 425 pM, 430 pM, 435 pM, 440 pM, 445 pM, 450 pM, 455 pM, 460 pM, 465 pM, 470 pM, 475 pM, 480 pM, 485 pM, 490 pM, 495 pM, or 500 pM. In some embodiments, the hGCGR EC₅₀ is measured in a functional assay described in “EXAMPLE 1: GCG Receptor Agonist Activity.”

In some embodiments, the molecule has a human glucagon-like peptide-1 receptor (“hGLP-1R”) EC₅₀:hGCGR EC₅₀ ratio of at least 30:1. In some embodiments, the molecule has a hGLP-1R EC₅₀:hGCGR EC₅₀ ratio of at least 40:1. In some embodiments, the molecule has a hGLP-1R EC₅₀:hGCGR EC₅₀ ratio of at least 50:1. In some embodiments, the molecule has a hGLP-1R EC₅₀:hGCGR EC₅₀ ratio of at least 60:1. In some embodiments, the molecule has a hGLP-1R EC₅₀:hGCGR EC₅₀ ratio of at least 70:1. In some embodiments, the molecule has a hGLP-1R EC₅₀:hGCGR EC₅₀ ratio of at least 80:1. In some embodiments, the molecule has a hGLP-1R EC₅₀:hGCGR EC₅₀ ratio of at least 90:1. In some embodiments, the molecule has a hGLP-1R EC₅₀:hGCGR EC₅₀ ratio of at least 100:1. In some embodiments, the molecule has a hGLP-1R EC₅₀:hGCGR EC₅₀ ratio of at least 150:1. In some embodiments, the molecule has a hGLP-1R EC₅₀:hGCGR EC₅₀ ratio of at least 200:1. In some embodiments, the molecule has a hGLP-1R EC₅₀:hGCGR EC₅₀ ratio of at least 250:1. In some embodiments, the molecule has a hGLP-1R EC₅₀:hGCGR EC₅₀ ratio of at least 300:1. In some embodiments, the molecule has a hGLP-1R EC₅₀:hGCGR EC₅₀ ratio of at least 350:1. In some embodiments, the molecule has a hGLP-1R EC₅₀:hGCGR EC₅₀ ratio of at least 400:1. In some embodiments, the molecule has a hGLP-1R EC₅₀:hGCGR EC₅₀ ratio of at least 450:1. In some embodiments, the molecule has a hGLP-1R EC₅₀:hGCGR EC₅₀ ratio of at least 500:1. In some embodiments, the molecule has a hGLP-1R EC₅₀:hGCGR EC₅₀ ratio of at least 550:1. In some embodiments, the molecule has a hGLP-1R EC₅₀:hGCGR EC₅₀ ratio of at least 650:1. In some embodiments, the molecule has a hGLP-1R EC₅₀:hGCGR EC₅₀ ratio of at least 700:1. In some embodiments, the molecule has a hGLP-1R EC₅₀:hGCGR EC₅₀ ratio of at least 750:1. In some embodiments, the molecule has a hGLP-1R EC₅₀:hGCGR EC₅₀ ratio of at least 800:1. In some embodiments, the molecule has a hGLP-1R EC₅₀:hGCGR EC₅₀ ratio of at least 850:1. In some embodiments, the molecule has a hGLP-1R EC₅₀:hGCGR EC₅₀ ratio of at least 900:1. In some embodiments, the molecule has a hGLP-1R EC₅₀:hGCGR EC₅₀ ratio of at least 950:1. In some embodiments, the molecule has a hGLP-1R EC₅₀:hGCGR EC₅₀ ratio of at least 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

In some embodiments, the molecule has a hGLP-1R EC₅₀:hGCGR EC₅₀ ratio of 30:1, 40:1, 45:1, 50:1, 55:1, 60:1, 65:1, 70:1, 75:1, 80:1, 85:1, 90:1, 95:1, 100:1, 125:1, 150:1, 175:1, 200:1, 225:1, 250:1, 275:1, 300:1, 325:1, 350:1, 375:1, 400:1, 425:1, 450:1, 475:1, 500:1, 525:1, 550:1, 575:1, 600:1, 625:1, 650:1, 675:1, 700:1, 725:1, 750:1, 775:1, 800:1, 825:1, 850:1, 875:1, 900:1, 925:1, 950:1, 975:1, or 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

Conjugates with ADNP Antibodies

Antibodies that specifically bind to 2,4 dinitrophenol (“DNP”) can be used as carrier immunoglobulins to improve one or more pharmacokinetic characteristics of a GCGR agonist provided herein. For example, in some embodiments, conjugation of a GCGR agonist to an aDNP antibody can prevent or mitigate in vivo degradation of the GCGR agonist by proteolysis or other in vivo activity-diminishing chemical modifications of the GCGR agonist, reduce renal clearance, enhance in vivo half-life or other pharmacokinetic properties of the GCGR agonist, such as, e.g., increasing the rate of absorption, reducing toxicity or immunogenicity, improving solubility, and/or increasing manufacturability or storage stability, compared to an unconjugated form of the GCGR agonist. Antibodies that specifically bind to DNP but have not been detected to bind to human proteins, cells, or tissues are described in WO 2010/108153, which is incorporated by reference herein.

Provided herein is a molecule that comprises a polypeptide that agonizes a glucagon receptor (“GCGR”), wherein: the polypeptide is selected from the polypeptides described herein; and an antibody that specifically binds to 2,4 dinitrophenol (“DNP”).

In some embodiments, the antibody is a monoclonal antibody, a recombinant antibody, a human antibody, a humanized antibody, or a chimeric antibody. In some embodiments, the antibody is a monoclonal antibody. In some embodiments, the antibody is a recombinant antibody. In some embodiments, the antibody is a human antibody. In some embodiments, the antibody is a humanized antibody. In some embodiments, the antibody is a chimeric antibody.

In some embodiments, the antibody is of the IgG1-, IgG2- IgG3-, or IgG4-type. In some embodiments, the antibody is of the IgG1-, IgG2-, or IgG4-subclass. In some embodiments, the antibody is of the IgG1- or IgG2-subclass.

In some embodiments, the antibody is of the IgG1-type. In some embodiments, the antibody is of the IgG2-type. In some embodiments, the antibody is of the IgG3-type. In some embodiments, the antibody is of the IgG4-type.

SEQ ID NOs have been assigned to variable light chain, variable heavy chain, light chain, heavy chain, CDRL1, CDRL2, CDRL3, CDRH1, CDRH2, and CDRH3 sequences of non-limiting example antibodies that specifically bind to DNP (“aDNP antibodies”) and are shown in Tables 3-8. The specific CDRs identified in Tables 5 and 6 are defined by Kabat. Each of the example anti-DNP heavy chains (H1-H10) listed in Table 8 can be combined with any of the example anti-DNP light chains shown in Table 7 to form an antibody.

In some embodiments, the aDNP antibody comprises at least one anti-DNP heavy chain and one anti-DNP light chain from those listed in Tables 7 and 8.

In some embodiments, the aDNP antibody comprises two different anti-DNP heavy chains and two different anti-DNP light chains listed in Tables 7 and 8. In other embodiments, the aDNP antibody comprises two identical light chains and two identical heavy chains.

In some embodiments, the aDNP antibody comprises two H1 heavy chains and two L1 light chains, or two H2 heavy chains and two L2 light chains, or two H3 heavy chains and two L3 light chains and other similar combinations of pairs of anti-DNP light chains and pairs of anti-DNP heavy chains.

In some embodiments, the aDNP antibody comprises a light chain variable (VH) region in which the full set of VL CDRs 1, 2, and 3 (combined) has at least 95% sequence identity to the VL CDRs 1, 2, and 3 of any one of SEQ ID NOs: 1688-1692. In some embodiments, the aDNP antibody comprises a light chain variable (VL) region in which the full set of VL CDRs 1, 2, and 3 (combined) has at least 95% sequence identity to the VL CDRs 1, 2, and 3 of any one of SEQ ID NOs: 1688-1692, wherein the VL CDRs 1, 2, and 3 are defined by any one of Kabat, Chothia, or IGMT. In some embodiments, the aDNP antibody comprises a light chain variable (VL) region in which the full set of VL CDRs 1, 2, and 3 (combined) has at least 95% sequence identity to the VL CDRs 1, 2, and 3 of any one of SEQ ID NOs: 1688-1692, wherein the VL CDRs 1, 2, and 3 are defined by Kabat. In some embodiments, the aDNP antibody comprises a light chain variable (VL) region in which the full set of VL CDRs 1, 2, and 3 (combined) has at least 95% sequence identity to the VL CDRs 1, 2, and 3 of any one of SEQ ID NOs: 1688-1692, wherein the VL CDRs 1, 2, and 3 are defined by Chothia. In some embodiments, the aDNP antibody comprises a light chain variable (VL) region in which the full set of VL CDRs 1, 2, and 3 (combined) has at least 95% sequence identity to the VL CDRs 1, 2, and 3 of any one of SEQ ID NOs: 1688-1692, wherein the VL CDRs 1, 2, and 3 are defined by IGMT.

In some embodiments, the aDNP antibody comprises a light chain variable (VH) region in which the full set of VL CDRs 1, 2, and 3 (combined) is identical to the VL CDRs 1, 2, and 3 of any one of SEQ ID NOs: 1688-1692. In some embodiments, the aDNP antibody comprises a light chain variable (VL) region in which the full set of VL CDRs 1, 2, and 3 (combined) is identical to the VL CDRs 1, 2, and 3 of any one of SEQ ID NOs: 1688-1692, wherein the VL CDRs 1, 2, and 3 are defined by any one of Kabat, Chothia, or IGMT. In some embodiments, the aDNP antibody comprises a light chain variable (VL) region in which the full set of VL CDRs 1, 2, and 3 (combined) is identical to the VL CDRs 1, 2, and 3 of any one of SEQ ID NOs: 1688-1692, wherein the VL CDRs 1, 2, and 3 are defined by Kabat. In some embodiments, the aDNP antibody comprises a light chain variable (VL) region in which the full set of VL CDRs 1, 2, and 3 (combined) is identical to the VL CDRs 1, 2, and 3 of any one of SEQ ID NOs: 1688-1692, wherein the VL CDRs 1, 2, and 3 are defined by Chothia. In some embodiments, the aDNP antibody comprises a light chain variable (VL) region in which the full set of VL CDRs 1, 2, and 3 (combined) is identical to the VL CDRs 1, 2, and 3 of any one of SEQ ID NOs: 1688-1692, wherein the VL CDRs 1, 2, and 3 are defined by IGMT.

In some embodiments, the aDNP antibody comprises a CDRL1, a CDRL2, and a CDRL3, wherein the CDRL1, the CDRL2, and the CDRL3 comprise amino acid sequences selected from:
i. SEQ ID NO: 1714, SEQ ID NO: 1718, and SEQ ID NO: 1720, respectively;
ii. SEQ ID NO: 1715, SEQ ID NO: 1718, and SEQ ID NO: 1721, respectively;
iii. SEQ ID NO: 1716, SEQ ID NO: 1718, and SEQ ID NO: 1722, respectively;
iv. SEQ ID NO: 1717, SEQ ID NO: 1719, and SEQ ID NO: 1723, respectively; or
v. SEQ ID NO: 1716, SEQ ID NO: 1718, and SEQ ID NO: 1724, respectively.

In some embodiments, the aDNP antibody comprises a heavy chain variable (VH) region in which the full set of VH CDRs 1, 2, and 3 (combined) has at least 95% sequence identity to the VH CDRs 1, 2, and 3 of any one of SEQ ID NOs: 1693-1697. In some embodiments, the aDNP antibody comprises a heavy chain variable (VH) region in which the full set of VH CDRs 1, 2, and 3 (combined) has at least 95% sequence identity to the VH CDRs 1, 2, and 3 of any one of SEQ ID NOs: 1693-1697, wherein the VH CDRs 1, 2, and 3 are defined by any one of Kabat, Chothia, or IGMT. In some embodiments, the aDNP antibody comprises a heavy chain variable (VH) region in which the full set of VH CDRs 1, 2, and 3 (combined) has at least 95% sequence identity to the VH CDRs 1, 2, and 3 of any one of SEQ ID NOs: 1693-1697, wherein the VH CDRs 1, 2, and 3 are defined by Kabat. In some embodiments, the aDNP antibody comprises a heavy chain variable (VH) region in which the full set of VH CDRs 1, 2, and 3 (combined) has at least 95% sequence identity to the VH CDRs 1, 2, and 3 of any one of SEQ ID NOs: 1693-1697, wherein the VH CDRs 1, 2, and 3 are defined by Chothia. In some embodiments, the aDNP antibody comprises a heavy chain variable (VH) region in which the full set of VH CDRs 1, 2, and 3 (combined) has at least 95% sequence identity to the VH CDRs 1, 2, and 3 of any one of SEQ ID NOs: 1693-1697, wherein the VH CDRs 1, 2, and 3 are defined by IGMT.

In some embodiments, the aDNP antibody comprises a heavy chain variable (VH) region in which the full set of VH CDRs 1, 2, and 3 (combined) are identical to the VH CDRs 1, 2, and 3 of any one of SEQ ID NOs: 1693-1697. In some embodiments, the aDNP antibody comprises a heavy chain variable (VH) region in which the full set of VH CDRs 1, 2, and 3 (combined) is identical to the VH CDRs 1, 2, and 3 of any one of SEQ ID NOs: 1693-1697, wherein the VH CDRs 1, 2, and 3 are defined by any one of Kabat, Chothia, or IGMT. In some embodiments, the aDNP antibody comprises a heavy chain variable (VH) region in which the full set of VH CDRs 1, 2, and 3 (combined) is identical to the VH CDRs 1, 2, and 3 of any one of SEQ ID NOs: 1693-1697, wherein the VH CDRs 1, 2, and 3 are defined by Kabat. In some embodiments, the aDNP antibody comprises a heavy chain variable (VH) region in which the full set of VH CDRs 1, 2, and 3 (combined) is identical to the VH CDRs 1, 2, and 3 of any one of SEQ ID NOs: 1693-1697, wherein the VH CDRs 1, 2, and 3 are defined by Chothia. In some embodiments, the aDNP antibody comprises a heavy chain variable (VH) region in which the full set of VH CDRs 1, 2, and 3 (combined) is identical to the VH CDRs 1, 2, and 3 of any one of SEQ ID NOs: 1693-1697, wherein the VH CDRs 1, 2, and 3 are defined by IGMT.

In some embodiments, the aDNP antibody comprises a CDRH1, a CDRH2, and a CDRH3, wherein the CDRH1, the CDRH2, and the CDRH3 comprise amino acid sequences selected from:
i. SEQ ID NO: 1725, SEQ ID NO: 1729, and SEQ ID NO: 1733, respectively;
ii. SEQ ID NO: 1726, SEQ ID NO: 1730, and SEQ ID NO: 1734, respectively;
iii. SEQ ID NO: 1726, SEQ ID NO: 1730, and SEQ ID NO: 1735, respectively;
iv. SEQ ID NO: 1726, SEQ ID NO: 1730, and SEQ ID NO: 1736, respectively;
v. SEQ ID NO: 1727, SEQ ID NO: 1731, and SEQ ID NO: 1737, respectively; or
vi. SEQ ID NO: 1728, SEQ ID NO: 1732, and SEQ ID NO: 1738, respectively.

In some embodiments, the aDNP antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise amino acid sequences of SEQ ID NO: 1715, SEQ ID NO: 1718, SEQ ID NO: 1721, SEQ ID NO: 1726, SEQ ID NO: 1730, SEQ ID NO: 1735, respectively.

In some embodiments, the aDNP antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise amino acid sequences of SEQ ID NO: 1716, SEQ ID NO: 1718, SEQ ID NO: 1722, SEQ ID NO: 1727, SEQ ID NO: 1731, and SEQ ID NO: 1737, respectively.

In some embodiments, the aDNP antibody comprises a light chain variable region, wherein the light chain variable region comprises an amino acid sequence selected from SEQ ID NOs: 1688-1962. In some embodiments, the light chain variable region comprises the amino acid sequence of SEQ ID NO: 1688. In some embodiments, the light chain variable region comprises the amino acid sequence of SEQ ID NO: 1689. In some embodiments, the light chain variable region comprises the amino acid sequence of SEQ ID NO: 1690. In some embodiments, the light chain variable region comprises the amino acid sequence of SEQ ID NO: 1691. In some embodiments, the light chain variable region comprises the amino acid sequence of SEQ ID NO: 1692.

In some embodiments, the aDNP antibody comprises a heavy chain variable region, wherein the heavy chain variable region comprises an amino acid sequence selected from SEQ ID NOs: 1693-1698. In some embodiments, the heavy chain variable region comprises the amino acid sequence of SEQ ID NO: 1693. In some embodiments, the heavy chain variable region comprises the amino acid sequence of SEQ ID NO: 1694. In some embodiments, the heavy chain variable region comprises the amino acid sequence of SEQ ID NO: 1695. In some embodiments, the heavy chain variable region comprises the amino acid sequence of SEQ ID NO: 1696. In some embodiments, the heavy chain variable region comprises the amino acid sequence of SEQ ID NO: 1697. In some embodiments, the heavy chain variable region comprises the amino acid sequence of SEQ ID NO: 1698.

In some embodiments, the aDNP antibody comprises a light chain variable region and a heavy chain variable region, wherein the light chain variable region and the heavy chain variable region comprise the amino acid sequences of SEQ ID NO: 1689 and SEQ ID NO: 1695, respectively.

In some embodiments, the aDNP antibody comprises a light chain variable (VL) region in which the full set of VL CDRs 1, 2, and 3 (combined) has at least 95% sequence identity to the VL CDRs 1, 2, and 3 of SEQ ID NO: 1689 and a heavy chain variable (VH) region in which the full set of VH CDRs 1, 2, and 3 (combined) has at least 95% sequence identity to the VH CDRs 1, 2, and 3 of SEQ ID NO: 1695. In some embodiments, the aDNP antibody comprises a VL region in which the full set of VL CDRs 1, 2, and 3 (combined) has at least 95% sequence identity to the VL CDRs 1, 2, and 3 of SEQ ID NO: 1689 and a VH region in which the full set of VH CDRs 1, 2, and 3 (combined) has at least 95% sequence identity to the VH CDRs 1, 2, and 3 of SEQ ID NO: 1695, wherein the VL CDRs 1, 2, and 3 and the VH CDRs 1, 2, and 3 are defined by any one of Kabat, Chothia, or IGMT.

In some embodiments, the aDNP antibody comprises a light chain variable (VL) region in which the full set of VL CDRs 1, 2, and 3 (combined) is identical to the VL CDRs 1, 2, and 3 of SEQ ID NO: 1689 and a heavy chain variable (VH) region in which the full set of VH CDRs 1, 2, and 3 (combined) is identical to the VH CDRs 1, 2, and 3 of SEQ ID NO: 1695. In some embodiments, the aDNP antibody comprises a VL region in which the full set of VL CDRs 1, 2, and 3 (combined) is identical to the VL CDRs 1, 2, and 3 of SEQ ID NO: 1689 and a VH region in which the full set of VH CDRs 1, 2, and 3 (combined) is identical to the VH CDRs 1, 2, and 3 of SEQ ID NO: 1695, wherein the VL CDRs 1, 2, and 3 and the VH CDRs 1, 2, and 3 are defined by any one of Kabat, Chothia, or IGMT. In some embodiments, the VL CDRs 1, 2, and 3 and the VH CDRs 1, 2, and 3 are defined by Kabat. In some embodiments, the VL CDRs 1, 2, and 3 and the VH CDRs 1, 2, and 3 are defined by Chothia. In some embodiments, the VL CDRs 1, 2, and 3 and the VH CDRs 1, 2, and 3 are defined by IGMT.

In some embodiments, the aDNP antibody comprises a light chain variable region and a heavy chain variable region, wherein the light chain variable region and the heavy chain variable region comprise the amino acid sequences of SEQ ID NO: 1690 and SEQ ID NO: 1697, respectively.

In some embodiments, the aDNP antibody comprises a light chain variable (VL) region in which the full set of VL CDRs 1, 2, and 3 (combined) has at least 95% sequence identity to the VL CDRs 1, 2, and 3 of SEQ ID NO: 1690 and a heavy chain variable (VH) region in which the full set of VH CDRs 1, 2, and 3 (combined) has at least 95% sequence identity to the VH CDRs 1, 2, and 3 of SEQ ID NO: 1697. In some embodiments, the aDNP antibody comprises a VL region in which the full set of VL CDRs 1, 2, and 3 (combined) has at least 95% sequence identity to the VL CDRs 1, 2, and 3 of SEQ ID NO: 1690 and a VH region in which the full set of VH CDRs 1, 2, and 3 (combined) has at least 95% sequence identity to the VH CDRs 1, 2, and 3 of SEQ ID NO: 1697, wherein the VL CDRs 1, 2, and 3 and the VH CDRs 1, 2, and 3 are defined by any one of Kabat, Chothia, or IGMT.

In some embodiments, the aDNP antibody comprises a light chain variable (VL) region in which the full set of VL CDRs 1, 2, and 3 (combined) is identical to the VL CDRs 1, 2, and 3 of SEQ ID NO: 1690 and a heavy chain variable (VH) region in which the full set of VH CDRs 1, 2, and 3 (combined) is identical to the VH CDRs 1, 2, and 3 of SEQ ID NO: 1697. In some embodiments, the aDNP antibody comprises a VL region in which the full set of VL CDRs 1, 2, and 3 (combined) is identical to the VL CDRs 1, 2, and 3 of SEQ ID NO: 1690 and a VH region in which the full set of VH CDRs 1, 2, and 3 (combined) is identical to the VH CDRs 1, 2, and 3 of SEQ ID NO: 1697, wherein the VL CDRs 1, 2, and 3 and the VH CDRs 1, 2, and 3 are defined by any one of Kabat, Chothia, or IGMT. In some embodiments, the VL CDRs 1, 2, and 3 and the VH CDRs 1, 2, and 3 are defined by Kabat. In some embodiments, the VL CDRs 1, 2, and 3 and the VH CDRs 1, 2, and 3 are defined by Chothia. In some embodiments, the VL CDRs 1, 2, and 3 and the VH CDRs 1, 2, and 3 are defined by IGMT.

In some embodiments, the aDNP antibody comprises a light chain, wherein the light chain comprises an amino acid sequence selected from SEQ ID NOs: 1699-1703. In some embodiments, the light chain comprises the amino acid sequence of SEQ ID NO: 1699. In some embodiments, the light chain comprises the amino acid sequence of SEQ ID NO: 1700. In some embodiments, the light chain comprises the amino acid sequence of SEQ ID NO: 1701. In some embodiments, the light chain comprises the amino acid sequence of SEQ ID NO: 1702. In some embodiments, the light chain comprises the amino acid sequence of SEQ ID NO: 1703.

In some embodiments, the aDNP antibody comprises a heavy chain, wherein the heavy chain comprises an amino acid sequence selected from SEQ ID NOs: 1704-1713. In some embodiments, the heavy chain comprises the amino acid sequence of SEQ ID NO: 1704. In some embodiments, the heavy chain comprises the amino acid sequence of SEQ ID NO: 1705. In some embodiments, the heavy chain comprises the amino acid sequence of SEQ ID NO: 1706. In some embodiments, the heavy chain comprises the amino acid sequence of SEQ ID NO: 1707. In some embodiments, the heavy chain comprises the amino acid sequence of SEQ ID NO: 1708. In some embodiments, the heavy chain comprises the amino acid sequence of SEQ ID NO: 1709. In some embodiments, the heavy chain comprises the amino acid sequence of SEQ ID NO: 1710. In some embodiments, the heavy chain comprises the amino acid sequence of SEQ ID NO: 1711. In some embodiments, the heavy chain comprises the amino acid sequence of SEQ ID NO: 1712. In some embodiments, the heavy chain comprises the amino acid sequence of SEQ ID NO: 1713.

In some embodiments, the aDNP antibody comprises a light chain and a heavy chain, wherein the light chain and heavy chain comprise the amino acid sequences of SEQ ID NO: 1700 and SEQ ID NO: 1712, respectively.

In some embodiments, the aDNP antibody comprises a light chain and a heavy chain, wherein the light chain and heavy chain comprise the amino acid sequences of SEQ ID NO: 1701 and SEQ ID NO: 1713, respectively.

In some embodiments, the aDNP antibody is conjugated, i.e., covalently bound, directly to an amino acid residue of the polypeptide agonist, or optionally, to a peptidyl or non-peptidyl linker moiety (including, but not limited to, aromatic or aryl linkers) that is covalently bound to an amino acid residue of the polypeptide agonist. For example, in some embodiments, an β-amino group of a lysine residue of the polypeptide is covalently linked to a C-terminus of a linker polypeptide; and an N-terminus of the linker polypeptide is conjugated to a cysteine residue of an aDNP antibody. Illustratively, in some embodiments, a lysine residue of the polypeptide agonist is covalently linked to a C-terminus of a linker polypeptide via an amide bond, e.g., an amide bond formed by condensation of an F amino group of a lysine residue and a carboxyl group of a C-terminus of a polypeptide linker. Additionally, in some embodiments, the N-terminus of the linker polypeptide is derivatized. For example, in some embodiments, the N-terminus of the linker polypeptide is acetylated, such as when a thioether linkage connects an acetylated N terminus of the linker polypeptide and a cysteine residue of the aDNP antibody, wherein the thioether linkage comprises a sulfur atom of the cysteine residue.

Alternatively, in some embodiments, the C-terminal amino acid residue of the polypeptide is covalently linked to the N-terminal amino acid residue of the linker polypeptide; and the C-terminal amino acid residue of the linker polypeptide is conjugated to a cysteine residue of an aDNP antibody. In some embodiments, the C-terminus of the linker polypeptide is derivatized. In some embodiments, the C-terminal amino acid residue of the linker polypeptide is modified for coupling to a cysteine residue (e.g., is bromoacetylated). Illustratively, in some embodiments, the C-terminus of the linker polypeptide is acetylated, such as when a thioether linkage connects an acetylated C-terminus of the linker polypeptide and a cysteine residue of the aDNP antibody, wherein the thioether linkage comprises a sulfur atom of the cysteine residue.

In some embodiments, the linker polypeptide is a linear polypeptide.

In some embodiments, the linker polypeptide comprises the amino acid sequence of any one of SEQ ID NOs: 1628-1683, 1739-1746, or 1841-1852. In some embodiments, the linker polypeptide comprises the amino acid sequence of any one of SEQ ID NOs: 1628-1683, 1739, 1850, or 1851. In some embodiments, the linker polypeptide comprises the amino acid sequence of any one of SEQ ID NOs: 1740-1746, 1841-1849, or 1852.

In some embodiments, the linker polypeptide comprises the amino acid sequence of any one of SEQ ID NOs: 1628-1683. In some embodiments, the linker polypeptide comprises the amino acid sequence of any one of SEQ ID NOs: 1628-1630. In some embodiments, the linker polypeptide comprises the amino acid sequence of SEQ ID NO: 1628. In some embodiments, the linker polypeptide comprises the amino acid sequence of SEQ ID NO: 1629. In some embodiments, the linker polypeptide comprises the amino acid sequence of SEQ ID NO: 1630. In some embodiments, the linker polypeptide comprises the amino acid sequence of SEQ ID NO: 1631.

TABLE 3
Variable Light (VL) Region Amino Acid Sequences of Example Anti-DNP Antibodies

| Designation | Clone | VL | SEQ ID NO: |
|---|---|---|---|
| L1 | 3A1 | DIQMTQSPSSVSASVGDRVTITCRASQGISNWLAWYQRKPGKAPKLLIYAASSLQS | 1688 |
| | | GVPSRFSGSGSGTDFTLTISSLQPEDFAAYYCQQASSFPWTFGQGTRVEIKRTV | |
| L2 | 3A4 | DIQMTQSPSSVSASVGDRVTITCRASQGISRRLAWYQQKPGKAPKLLIYAASSLQS | 1689 |
| | | GVPSRFSGSGSGTDFTLTISSLQPEDFATYYCQQANSFPFTFGPGTKVDIKRTV | |
| L3 | 3B1 | DIQMTQSPSSLSASEGDRVTITCRASQGIRNDLGWYQQKPGKAPKRLIYAASSLQS | 1690 |
| | | GVPLRFSGSGSGTEFTLTISSLQPEDFATYYCLQYNSYPWTFGQGTKVEIKRTV | |
| L4 | 3C2 | DIQMTQSPSSLSASVGDRVTITCRASQGMSNYLAWYQQKPRKVPKLLIYAASTLQ | 1691 |
| | | SGVPSRFSGSGSGTDFTLTISSLQPEDVATYYCQKFNSAPFTFGPGTKVDIKRTV | |
| L5 | 3H4 | DIQMTLSPSSLSASVGDRVTITCRASQGIRNDLGWYQQKPGKAPKRLIYAASSLQS | 1692 |
| | | GVPSRFSGSGSGTEFTLTISSLQPEDFATYYCLQYNSSPWTFGQGTEVEIKRTV | |

TABLE 4
Variable Heavy (VH) Region Amino Acid Sequences of Example Anti-DNP Antibodies

| Designation | Clone | VH | SEQ ID NO: |
|---|---|---|---|
| H1 | 3A1 | QVQLQESGPGLVKPSETLSLTCTVSGGSISHYYWSWIRQPPGKGLGWIGYIYYSGS | 1693 |
| | | TNYNPSLKSRVTISVDTSKNQFSLKLTSVTAADTAVYYCARARGDGYNYPDAFDI | |
| | | WGQGTMVTVSS | |
| H2 | 3A4 | QVQLVESGGGVVQPGRSLRLSCAASGFTFSSYGMHWVRQAPGKGLEWVAVIWY | 1694 |
| | 3C2 | DGSNKYYADSVKGRFTISRDNSKNTLYLQMNSLRAEDTAVYYCARYNWNYGMD | |
| | | VWGQGTTVTVSS | |
| H3 | 3A4-F | QVQLVESGGGVVQPGRSLRLSCAASGFTFSSYGMHWVRQAPGKGLEWVAVIWY | 1695 |
| | (W101F) | DGSNKYYADSVKGRFTISRDNSKNTLYLQMNSLRAEDTAVYYCARYNFNYGMD | |
| | | VWGQGTTVTVSS | |
| H4 | 3A4-Y | QVQLVESGGGVVQPGRSLRLSCAASGFTFSSYGMHWVRQAPGKGLEWVAVIWY | 1696 |
| | (W101Y) | DGSNKYYADSVKGRFTISRDNSKNTLYLQMNSLRAEDTAVYYCARYNYNYGMD | |
| | | VWGQGTTVTVSS | |
| H5 | 3A4-FSS | QVQLVESGGGVVQPGRSLRLSCAASGFTFSSYGMHWVRQAPGKGLEWVAVIWY | 1695 |
| | | DGSNKYYADSVKGRFTISRDNSKNTLYLQMNSLRAEDTAVYYCARYNFNYGMD | |
| | | VWGQGTTVTVSS | |
| H6 | 3A4-YSS | QVQLVESGGGVVQPGRSLRLSCAASGFTFSSYGMHWVRQAPGKGLEWVAVIWY | 1696 |
| | | DGSNKYYADSVKGRFTISRDNSKNTLYLQMNSLRAEDTAVYYCARYNYNYGMD | |
| | | VWGQGTTVTVSS | |
| H7 | 3B1 | QVQLQESGPGLVKPSETLSLTCTVSGGSISSYYWSWIRQPPGKGLEWIGYIYYSGN | 1697 |
| | | TNSNPSLKSRVTISVDTSKNQFSLKLSSVTAADTAVYYCARTYYDSSGYYYRAFDI | |
| | | WGQGTMVTVSS | |
| H8 | 3H4 | QVQLQESGPGLVKPLQTLSLTCTVSGGSISSGGYYWSWIRQHPGKGLEWIGYIYYS | 1698 |
| | | RSTYYNPSLKSRVTISVDTSKNQFSLKLSSVTAADTAVYYCARTGYSSGWYPFDY | |
| | | WGQGTLVTVSS | |
| H9 | 3A4 SEFL2 | QVQLVESGGGVVQPGRSLRLSCAASGFTFSSYGMHWVRQAPGKGLEWVAVIWY | 1695 |
| | E384C | DGSNKYYADSVKGRFTISRDNSKNTLYLQMNSLRAEDTAVYYCARYNFNYGMD | |
| | | VWGQGTTVTVSS | |
| H10 | 3B1 SEFL2 | QVQLQESGPGLVKPSETLSLTCTVSGGSISSYYWSWIRQPPGKGLEWIGYIYYSGN | 1697 |
| | E384C | TNSNPSLKSRVTISVDTSKNQFSLKLSSVTAADTAVYYCARTYYDSSGYYYRAFDI | |
| | | WGQGTMVTVSS | |

TABLE 5
CDRL1, CDRL2, and CDRL3 Amino Acid Sequences of Example Anti-DNP Antibodies

| Designation | Clone | CDRL1 | CDRL2 | CDRL3 |
|---|---|---|---|---|
| L1 | 3A1 | RASQGISNWLA | AASSLQS | QQASSFPWT |
| | | SEQ ID NO: 1714 | SEQ ID NO: 1718 | SEQ ID NO: 1720 |
| L2 | 3A4 | RASQGISRRLA | AASSLQS | QQANSFPFT |
| | | SEQ ID NO: 1715 | SEQ ID NO: 1718 | SEQ ID NO: 1721 |
| L3 | 3B1 | RASQGIRNDLG | AASSLQS | LQYNSYPWT |
| | | SEQ ID NO: 1716 | SEQ ID NO: 1718 | SEQ ID NO: 1722 |
| L4 | 3C2 | RASQGMSNYLA | AASTLQS | QKFNSAPFT |
| | | SEQ ID NO: 1717 | SEQ ID NO: 1719 | SEQ ID NO: 1723 |
| L5 | 3H4 | RASQGIRNDLG | AASSLQS | LQYNSSPWT |
| | | SEQ ID NO: 1716 | SEQ ID NO: 1718 | SEQ ID NO: 1724 |

TABLE 6
CDRH1, CDRH2, and CDRH3 Amino Acid Sequences of Example Anti-DNP Antibodies

| Designation | Clone | CDRH1 | CDRH2 | CDRH3 |
|---|---|---|---|---|
| H1 | 3A1 | HYYWS | YIYYSGSTNYNPSLKS | ARGDGYNYPDAFDI |
| | | SEQ ID NO: 1725 | SEQ ID NO: 1729 | SEQ ID NO: 1733 |
| H2 | 3A4 | SYGMH | VIWYDGSNKYYADSVKG | YNWNYGMDV |
| | 3C2 | SEQ ID NO: 1726 | SEQ ID NO: 1730 | SEQ ID NO: 1734 |
| H3 | 3A4-F | SYGMH | VIWYDGSNKYYADSVKG | YNFNYGMDV |
| | (W101F) | SEQ ID NO: 1726 | SEQ ID NO: 1730 | SEQ ID NO: 1735 |
| H4 | 3A4-Y | SYGMH | VIWYDGSNKYYADSVKG | YNYNYGMDV |
| | (W101Y) | SEQ ID NO: 1726 | SEQ ID NO: 1730 | SEQ ID NO: 1736 |
| H5 | 3A4-FSS | SYGMH | VIWYDGSNKYYADSVKG | YNFNYGMDV |
| | | SEQ ID NO: 1726 | SEQ ID NO: 1730 | SEQ ID NO: 1735 |
| H6 | 3A4-YSS | SYGMH | VIWYDGSNKYYADSVKG | YNYNYGMDV |
| | | SEQ ID NO: 1726 | SEQ ID NO: 1730 | SEQ ID NO: 1736 |
| H7 | 3B1 | SYYWS | YIYYSGNTNSNPSLKS | TYYDSSGYYYRAFDI |
| | | SEQ ID NO: 1727 | SEQ ID NO: 1731 | SEQ ID NO: 1737 |
| H8 | 3H4 | SGGYYWS | YIYYSRSTYYNPSLKS | TGYSSGWYPFDY |
| | | SEQ ID NO: 1728 | SEQ ID NO: 1732 | SEQ ID NO: 1738 |
| H9 | 3A4-F SEFL2 | SYGMH | VIWYDGSNKYYADSVKG | YNFNYGMDV |
| | E384C | SEQ ID NO: 1726 | SEQ ID NO: 1730 | SEQ ID NO: 1735 |
| H10 | 3B1 SEFL2 | SYYWS | YIYYSGNTNSNPSLKS | TYYDSSGYYYRAFDI |
| | E384C | SEQ ID NO: 1727 | SEQ ID NO: 1731 | SEQ ID NO: 1737 |

TABLE 7
Light Chain (LC) Amino Acid Sequences of Example Anti-DNP Antibodies

| Designation | Clone | LC | SEQ ID NO: |
|---|---|---|---|
| L1 | 3A1 | DIQMTQSPSSVSASVGDRVTITCRASQGISNWLAWYQRKPGKAPKLLIYAASSLQS | 1699 |
| | | GVPSRFSGSGSGTDFTLTISSLQPEDFAAYYCQQASSFPWTFGQGTRVEIKRTVAA | |
| | | PSVFIFPPSDEQLKSGTASVVCLLNNFYPREAKVQWKVDNALQSGNSQESVTEQD | |
| | | SKDSTYSLSSTLTLSKADYEKHKVYACEVTHQGLSSPVTKSFNRGEC | |
| L2 | 3A4 | DIQMTQSPSSVSASVGDRVTITCRASQGISRRLAWYQQKPGKAPKLLIYAASSLQS | 1700 |
| | | GVPSRFSGSGSGTDFTLTISSLQPEDFATYYCQQANSFPFTFGPGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| L3 | 3B1 | DIQMTQSPSSLSASEGDRVTITCRASQGIRNDLGWYQQKPGKAPKRLIYAASSLQS | 1701 |
| | | GVPLRFSGSGSGTEFTLTISSLQPEDFATYYCLQYNSYPWTFGQGTKVEIKRTVAA | |
| | | PSVFIFPPSDEQLKSGTASVVCLLNNFYPREAKVQWKVDNALQSGNSQESVTEQD | |
| | | SKDSTYSLSSTLTLSKADYEKHKVYACEVTHQGLSSPVTKSFNRGEC | |
| L4 | 3C2 | DIQMTQSPSSLSASVGDRVTITCRASQGMSNYLAWYQQKPRKVPKLLIYAASTLQ | 1702 |
| | | SGVPSRFSGSGSGTDFTLTISSLQPEDVATYYCQKFNSAPFTFGPGTKVDIKRTVAA | |
| | | PSVFIFPPSDEQLKSGTASVVCLLNNFYPREAKVQWKVDNALQSGNSQESVTEQD | |
| | | SKDSTYSLSSTLTLSKADYEKHKVYACEVTHQGLSSPVTKSFNRGEC | |
| L5 | 3H4 | DIQMTLSPSSLSASVGDRVTITCRASQGIRNDLGWYQQKPGKAPKRLIYAASSLQS | 1703 |
| | | GVPSRFSGSGSGTEFTLTISSLQPEDFATYYCLQYNSSPWTFGQGTEVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |

TABLE 8
Heavy Chain (HC) Amino Acid Sequences of Example Anti-DNP Antibodies

| Designation | Clone | HC | SEQ ID NO: |
|---|---|---|---|
| H1 | 3A1 | QVQLQESGPGLVKPSETLSLTCTVSGGSISHYYWSWIRQPPGKGLGWIGYIYYSGS | 1704 |
| | | TNYNPSLKSRVTISVDTSKNQFSLKLTSVTAADTAVYYCARARGDGYNYPDAFDI | |
| | | WGQGTMVTVSSASTKGPSVFPLAPCSRSTSESTAALGCLVKDYFPEPVTVSWNSG | |
| | | ALTSGVHTFPAVLQSSGLYSLSSVVTVPSSNFGTQTYTCNVDHKPSNTKVDKTVE | |
| | | RKCCVECPPCPAPPVAGPSVFLFPPKPKDTLMISRTPEVTCVVVDVSHEDPEVQFN | |
| | | WYVDGVEVHNAKTKPREEQFNSTFRVVSVLTVVHQDWLNGKEYKCKVSNKGLP | |
| | | APIEKTISKTKGQPREPQVYTLPPSREEMTKNQVSLTCLVKGFYPSDIAVEWESNG | |
| | | QPENNYKTTPPMLDSDGSFFLYSKLTVDKSRWQQGNVFSCSVMHEALHNHYTQK | |
| | | SLSLSPG(K) | |
| H2 | 3A4 | QVQLVESGGGVVQPGRSLRLSCAASGFTFSSYGMHWVRQAPGKGLEWVAVIWY | 1705 |
| | 3C2 | DGSNKYYADSVKGRFTISRDNSKNTLYLQMNSLRAEDTAVYYCARYNWNYGMD | |
| | | VWGQGTTVTVSSASTKGPSVFPLAPCSRSTSESTAALGCLVKDYFPEPVTVSWNS | |
| | | GALTSGVHTFPAVLQSSGLYSLSSVVTVPSSNFGTQTYTCNVDHKPSNTKVDKTV | |
| | | ERKCCVECPPCPAPPVAGPSVFLFPPKPKDTLMISRTPEVTCVVVDVSHEDPEVQF | |
| | | NWYVDGVEVHNAKTKPREEQFNSTFRVVSVLTVVHQDWLNGKEYKCKVSNKG | |
| | | LPAPIEKTISKTKGQPREPQVYTLPPSREEMTKNQVSLTCLVKGFYPSDIAVEWESN | |
| | | GQPENNYKTTPPMLDSDGSFFLYSKLTVDKSRWQQGNVFSCSVMHEALHNHYTQ | |
| | | KSLSLSPG(K) | |
| H3 | 3A4-F | QVQLVESGGGVVQPGRSLRLSCAASGFTFSSYGMHWVRQAPGKGLEWVAVIWY | 1706 |
| | (W101F) | DGSNKYYADSVKGRFTISRDNSKNTLYLQMNSLRAEDTAVYYCARYNFNYGMD | |
| | | VWGQGTTVTVSSASTKGPSVFPLAPCSRSTSESTAALGCLVKDYFPEPVTVSWNS | |
| | | GALTSGVHTFPAVLQSSGLYSLSSVVTVPSSNFGTQTYTCNVDHKPSNTKVDKTV | |
| | | ERKCCVECPPCPAPPVAGPSVFLFPPKPKDTLMISRTPEVTCVVVDVSHEDPEVQF | |
| | | NWYVDGVEVHNAKTKPREEQFNSTFRVVSVLTVVHQDWLNGKEYKCKVSNKG | |
| | | LPAPIEKTISKTKGQPREPQVYTLPPSREEMTKNQVSLTCLVKGFYPSDIAVEWESN | |
| | | GQPENNYKTTPPMLDSDGSFFLYSKLTVDKSRWQQGNVFSCSVMHEALHNHYTQ | |
| | | KSLSLSPG(K) | |
| H4 | 3A4-Y | QVQLVESGGGVVQPGRSLRLSCAASGFTFSSYGMHWVRQAPGKGLEWVAVIWY | 1707 |
| | (W101Y) | DGSNKYYADSVKGRFTISRDNSKNTLYLQMNSLRAEDTAVYYCARYNYNYGMD | |
| | | VWGQGTTVTVSSASTKGPSVFPLAPCSRSTSESTAALGCLVKDYFPEPVTVSWNS | |
| | | GALTSGVHTFPAVLQSSGLYSLSSVVTVPSSNFGTQTYTCNVDHKPSNTKVDKTV | |
| | | ERKCCVECPPCPAPPVAGPSVFLFPPKPKDTLMISRTPEVTCVVVDVSHEDPEVQF | |
| | | NWYVDGVEVHNAKTKPREEQFNSTFRVVSVLTVVHQDWLNGKEYKCKVSNKG | |
| | | LPAPIEKTISKTKGQPREPQVYTLPPSREEMTKNQVSLTCLVKGFYPSDIAVEWESN | |
| | | GQPENNYKTTPPMLDSDGSFFLYSKLTVDKSRWQQGNVFSCSVMHEALHNHYTQ | |
| | | KSLSLSPG(K) | |
| H5 | 3A4-FSS | QVQLVESGGGVVQPGRSLRLSCAASGFTFSSYGMHWVRQAPGKGLEWVAVIWY | 1708 |
| | | DGSNKYYADSVKGRFTISRDNSKNTLYLQMNSLRAEDTAVYYCARYNFNYGMD | |
| | | VWGQGTTVTVSSASTKGPSVFPLAPCSRSTSESTAALGCLVKDYFPEPVTVSWNS | |
| | | GALTSGVHTFPAVLQSSGLYSLSSVVTVPSSNFGTQTYTCNVDHKPSNTKVDKTV | |
| | | ERKSSVECPPCPAPPVAGPSVFLFPPKPKDTLMISRTPEVTCVVVDVSHEDPEVQFN | |
| | | WYVDGVEVHNAKTKPREEQFNSTFRVVSVLTVVHQDWLNGKEYKCKVSNKGLP | |
| | | APIEKTISKTKGQPREPQVYTLPPSREEMTKNQVSLTCLVKGFYPSDIAVEWESNG | |
| | | QPENNYKTTPPMLDSDGSFFLYSKLTVDKSRWQQGNVFSCSVMHEALHNHYTQK | |
| | | SLSLSPG(K) | |
| H6 | 3A4-YSS | QVQLVESGGGVVQPGRSLRLSCAASGFTFSSYGMHWVRQAPGKGLEWVAVIWY | 1709 |
| | | DGSNKYYADSVKGRFTISRDNSKNTLYLQMNSLRAEDTAVYYCARYNYNYGMD | |
| | | VWGQGTTVTVSSASTKGPSVFPLAPCSRSTSESTAALGCLVKDYFPEPVTVSWNS | |
| | | GALTSGVHTFPAVLQSSGLYSLSSVVTVPSSNFGTQTYTCNVDHKPSNTKVDKTV | |
| | | ERKSSVECPPCPAPPVAGPSVFLFPPKPKDTLMISRTPEVTCVVVDVSHEDPEVQFN | |
| | | WYVDGVEVHNAKTKPREEQFNSTFRVVSVLTVVHQDWLNGKEYKCKVSNKGLP | |
| | | APIEKTISKTKGQPREPQVYTLPPSREEMTKNQVSLTCLVKGFYPSDIAVEWESNG | |
| | | QPENNYKTTPPMLDSDGSFFLYSKLTVDKSRWQQGNVFSCSVMHEALHNHYTQK | |
| | | SLSLSPG(K) | |
| H7 | 3B1 | QVQLQESGPGLVKPSETLSLTCTVSGGSISSYYWSWIRQPPGKGLEWIGYIYYSGN | 1710 |
| | | TNSNPSLKSRVTISVDTSKNQFSLKLSSVTAADTAVYYCARTYYDSSGYYYRAFDI | |
| | | WGQGTMVTVSSASTKGPSVFPLAPCSRSTSESTAALGCLVKDYFPEPVTVSWNSG | |
| | | ALTSGVHTFPAVLQSSGLYSLSSVVTVPSSNFGTQTYTCNVDHKPSNTKVDKTVE | |
| | | RKCCVECPPCPAPPVAGPSVFLFPPKPKDTLMISRTPEVTCVVVDVSHEDPEVQFN | |
| | | WYVDGVEVHNAKTKPREEQFNSTFRVVSVLTVVHQDWLNGKEYKCKVSNKGLP | |
| | | APIEKTISKTKGQPREPQVYTLPPSREEMTKNQVSLTCLVKGFYPSDIA VEWESNG | |
| | | QPENNYKTTPPMLDSDGSFFLYSKLTVDKSRWQQGNVFSCSVMHEALHNHYTQK | |
| | | SLSLSPG(K) | |
| H8 | 3H4 | QVQLQESGPGLVKPLQTLSLTCTVSGGSISSGGYYWSWIRQHPGKGLEWIGYIYYS | 1711 |
| | | RSTYYNPSLKSRVTISVDTSKNQFSLKLSSVTAADTAVYYCARTGYSSGWYPFDY | |
| | | WGQGTLVTVSSASTKGPSVFPLAPCSRSTSESTAALGCLVKDYFPEPVTVSWNSG | |
| | | ALTSGVHTFPAVLQSSGLYSLSSVVTVPSSNFGTQTYTCNVDHKPSNTKVDKTVE | |
| | | RKCCVECPPCPAPPVAGPSVFLFPPKPKDTLMISRTPEVTCVVVDVSHEDPEVQFN | |
| | | WYVDGVEVHNAKTKPREEQFNSTFRVVSVLTVVHQDWLNGKEYKCKVSNKGLP | |
| | | APIEKTISKTKGQPREPQVYTLPPSREEMTKNQVSLTCLVKGFYPSDIAVEWESNG | |
| | | QPENNYKTTPPMLDSDGSFFLYSKLTVDKSRWQQGNVFSCSVMHEALHNHYTQK | |
| | | SLSLSPG(K) | |
| H9 | 3A4-F | QVQLVESGGGVVQPGRSLRLSCAASGFTFSSYGMHWVRQAPGKGLEWVAVIWY | 1712 |
| | SEFL2 | DGSNKYYADSVKGRFTISRDNSKNTLYLQMNSLRAEDTAVYYCARYNFNYGMD | |
| | E384C | VWGQGTTVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPC | |
| | | VKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPG(K) | |
| H10 | 3B1 SEFL2 | QVQLQESGPGLVKPSETLSLTCTVSGGSISSYYWSWIRQPPGKGLEWIGYIYYSGN | 1713 |
| | E384C | TNSNPSLKSRVTISVDTSKNQFSLKLSSVTAADTAVYYCARTYYDSSGYYYRAFDI | |
| | | WGQGTMVTVSSASTKGPSVFPLAPSSKSTSGGTAALGCLVKDYFPEPVTVSWNSG | |
| | | ALTSGVHTFPAVLQSSGLYSLSSVVTVPSSSLGTQTYICNVNHKPSNTKVDKKVEP | |
| | | KSCDKTHTCPPCPAPELLGGPSVFLFPPKPKDTLMISRTPEVTCVVVDVSHEDPCV | |
| | | KFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEW | |
| | | ESNGQPENNYKTTPPVLDSDGSFFLYSKLTVDKSRWQQGNVFSCSVMHEALHNH | |
| | | YTQKSLSLSPG(K) | |

TABLE S1
Amino Acid Sequences of Example Half-Life Extending Domains

| Description | Amino Acid Sequence | SEQ ID NO: |
|---|---|---|
| 655-341 SEFL2 | QVQLQESGPGLVKPSQTLSLTCTVSGGSISSGDYFWSWIRQLPGKGLEWIGHIHNSGTTYY | 1853 |
| T487C HC | NPSLKSRVTISVDTSKKQFSLRLSSVTAADTAVYYCARDRGGDYAYGMDVWGQGTTVTV | |
| | SSASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHTCPPCPAPELLGG | |
| | PSVFLFPPKPKDTLMISRTPEVTCVVVDVSHEDPEVKFNWYVDGVEVHNAKTKPCEEQYG | |
| | STYRCVSVLTVLHQDWLNGKEYKCKVSNKALPAPIEKTISKAKGQPREPQVYTLPPSREE | |
| | MCKNQVSLTCLVKGFYPSDIAVEWESNGQPENNYKTTPPVLDSDGSFFLYSKLTVDKSRW | |
| | QQGNVFSCSVMHEALHNHYTQKSLSLSPGK | |
| 655-341 SEFL2 | QVQLQESGPGLVKPSQTLSLTCTVSGGSISSGDYFWSWIRQLPGKGLEWIGHIHNSGTTYY | 1854 |
| E384C HC | NPSLKSRVTISVDTSKKQFSLRLSSVTAADTAVYYCARDRGGDYAYGMDVWGQGTTVTV | |
| | SSASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHTCPPCPAPELLGG | |
| | PSVFLFPPKPKDTLMISRTPEVTCVVVDVSHEDPCVKFNWYVDGVEVHNAKTKPCEEQYG | |
| | STYRCVSVLTVLHQDWLNGKEYKCKVSNKALPAPIEKTISKAKGQPREPQVYTLPPSREE | |
| | MTKNQVSLTCLVKGFYPSDIAVEWESNGQPENNYKTTPPVLDSDGSFFLYSKLTVDKSRW | |
| | QQGNVFSCSVMHEALHNHYTQKSLSLSPGK | |
| 655-341 SEFL2 | QVQLQESGPGLVKPSQTLSLTCTVSGGSISSGDYFWSWIRQLPGKGLEWIGHIHNSGTTYY | 1855 |
| D88C HC | NPSLKSRVTISVDTSKKQFSLRLSSVTAADTAVYYCARDRGGDYAYGMDVWGQGTTVTV | |
| | SSASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHTCPPCPAPELLGG | |
| | PSVFLFPPKPKDTLMISRTPEVTCVVVDVSHEDPEVKFNWYVDGVEVHNAKTKPCEEQYG | |
| | STYRCVSVLTVLHQDWLNGKEYKCKVSNKALPAPIEKTISKAKGQPREPQVYTLPPSREE | |
| | MTKNQVSLTCLVKGFYPSDIAVEWESNGQPENNYKTTPPVLDSDGSFFLYSKLTVDKSRW | |
| | QQGNVFSCSVMHEALHNHYTQKSLSLSPGK | |
| 655-341 SEFL2 | EIVLTQSPGTLSLSPGERATLSCRASQGISRSELAWYQQKPGQAPSLLIYGASSRATGIPDRF | 1856 |
| T487C LC | SGSGSGTDFTLTISRLEPEDFAVYYCQQFGSSPWTFGQGTKVEIKRTVAAPSVFIFPPSDEQL | |
| 655-341 SEFL2 | KSGTASVVCLLNNFYPREAKVQWKVDNALQSGNSQESVTEQDSKDSTYSLSSTLTLSKAD | |
| E384C LC | YEKHKVYACEVTHQGLSSPVTKSFNRGEC | |
| 655-341 SEFL2 | EIVLTQSPGTLSLSPGERATLSCRASQGISRSELAWYQQKPGQAPSLLIYGASSRATGIPDRF | 1857 |
| D88C LC | SGSGSGTCFTLTISRLEPEDFAVYYCQQFGSSPWTFGQGTKVEIKRTVAAPSVFIFPPSDEQL | |
| | KSGTASVVCLLNNFYPREAKVQWKVDNALQSGNSQESVTEQDSKDSTYSLSSTLTLSKAD | |
| | YEKHKVYACEVTHQGLSSPVTKSFNRGEC | |
| Fc SEFL2 T487C | DKTHTCPPCPAPELLGGPSVFLFPPKPKDTLMISRTPEVTCVVVDVSHEDPEVKFNWYVDG | 1858 |
| | VEVHNAKTKPCEEQYGSTYRCVSVLTVLHQDWLNGKEYKCKVSNKALPAPIEKTISKAK | |
| | GQPREPQVYTLPPSREEMCKNQVSLTCLVKGFYPSDIAVEWESNGQPENNYKTTPPVLDS | |
| | DGSFFLYSKLTVDKSRWQQGNVFSCSVMHEALHNHYTQKSLSLSPGK | |

Conjugates with Glucose-Dependent Insulinotropic Polypeptide Receptor Antagonists

The present disclosure further provides molecules in which a glucagon (SEQ ID NO: 1576) or glucagon analog, including, but not limited to, a polypeptide agonist described above, is conjugated to an antigen-binding protein that specifically binds to a glucose-dependent insulinotropic polypeptide receptor (“GIPR”), such as an anti-GIPR antibody.

Provided herein is a molecule comprising: a first polypeptide that agonizes a glucagon receptor (“GCGR”); and an antigen-binding protein that specifically binds to a glucose-dependent insulinotropic polypeptide receptor (“GIPR”).

In some embodiments, the antigen-binding protein (e.g., the anti-GIPR antibody) binds to the extracellular portion of human GIPR. In some embodiments, the antigen-binding protein inhibits binding of GIP to the extracellular portion of human GIPR. In some embodiments, the human GIPR has the amino acid sequence of SEQ ID NO: 1577. In some embodiments, the human GIPR has the amino acid sequence of SEQ ID NO: 1578. In some embodiments, the human GIPR has the amino acid sequence of SEQ ID NO: 1579.

In some embodiments, the antigen-binding protein (e.g., the anti-GIPR antibody) binds to the N-terminal extracellular domain of human GIPR. In some embodiments, the antigen-binding protein inhibits binding of GIP to the N-terminal extracellular domain of human GIPR. In some embodiments, the human GIPR has the amino acid sequence of SEQ ID NO: 1577. In some embodiments, the human GIPR has the amino acid sequence of SEQ ID NO: 1578. In some embodiments, the human GIPR has the amino acid sequence of SEQ ID NO: 1579.

In some embodiments, the antigen-binding protein (e.g., the anti-GIPR antibody) binds to one or more amino acids at positions 1-139 of human GIPR. In some embodiments, the antigen-binding protein (e.g., the anti-GIPR antibody) inhibits binding of GIP to the amino acids at positions 1-139 of human GIPR. In some embodiments, the human GIPR has the amino acid sequence of SEQ ID NO: 1577. In some embodiments, the human GIPR has the amino acid sequence of SEQ ID NO: 1578. In some embodiments, the human GIPR has the amino acid sequence of SEQ ID NO: 1579.

In some embodiments, the antigen-binding protein is an antagonist of GIPR. In some embodiments, the antigen-binding protein is an antagonist of human GIPR. In some embodiments, the human GIPR has the amino acid sequence of SEQ ID NO: 1577. In some embodiments, the human GIPR has the amino acid sequence of SEQ ID NO: 1578. In some embodiments, the human GIPR has the amino acid sequence of SEQ ID NO: 1579.

In some embodiments, the antigen-binding protein (e.g., the anti-GIPR antibody) is an antagonist of human GIPR and inhibits binding of GIP to the N-terminal extracellular domain of human GIPR. In some embodiments, the antigen-binding protein (e.g., the anti-GIPR antibody) is an antagonist of human GIPR and inhibits binding of GIP to the amino acids at positions 1-139 of human GIPR. In some embodiments, the human GIPR has the amino acid sequence of SEQ ID NO: 1577. In some embodiments, the human GIPR has the amino acid sequence of SEQ ID NO: 1578. In some embodiments, the human GIPR has the amino acid sequence of SEQ ID NO: 1579.

In some embodiments, the antigen-binding protein (e.g., the anti-GIPR antibody) is an antagonist of human GIPR and specifically binds to the N-terminal extracellular domain of human GIPR. In some embodiments, the antigen-binding protein (e.g., the anti-GIPR antibody) is an antagonist of human GIPR and specifically binds to one or more of the amino acids at positions 1-139 of human GIPR. In some embodiments, the human GIPR has the amino acid sequence of SEQ ID NO: 1577. In some embodiments, the human GIPR has the amino acid sequence of SEQ ID NO: 1578. In some embodiments, the human GIPR has the amino acid sequence of SEQ ID NO: 1579.

Non-limiting examples of GIPR antagonists are provided in, for example, WO 2017/112824.

Non-limiting example antigen-binding proteins that specifically bind to GIPR, including human GIPR (hGIPR), are described herein. In some embodiments, the human GIPR has the amino acid sequence of SEQ ID NO: 1577. In some embodiments, the human GIPR has the amino acid sequence of SEQ ID NO: 1578. In some embodiments, the human GIPR has the amino acid sequence of SEQ ID NO: 1579.

The non-limiting example antigen-binding proteins described herein are antagonists of GIPR and can have one, two, three, four, five, six, seven, or all eight of the following characteristics:
a) ability to prevent or reduce binding of GIP to GIPR, where the levels can be measured, for example, by the methods such as radioactive- or fluorescence-labeled ligand binding study, or by the methods described herein (e.g. a cAMP assay or another functional assay). The decrease can be at least 10%, 25%, 50%, 100% or more relative to the pre-treatment levels of SEQ ID NO: 1577, 1578, or 1579 under comparable conditions;
b) ability to decrease body weight or reduce body weight gain;
c) ability to decrease fat mass or decrease inflammation in fat tissue;
d) ability to decrease circulating cholesterol levels;
e) ability to decrease circulating triglyceride levels;
f) ability to decrease liver steatosis or reduce triglyceride level in liver;
g) decrease AST, ALT, and/or ALP levels.

In some embodiments, the antigen-binding protein has one or more of the following activities:
a) binds human GIPR such that KD is ≤200 nM, is ≤150 nM, is ≤100 nM, is ≤50 nM, is ≤10 nM, is ≤5 nM, is ≤2 nM, or is ≤1 nM, e.g., as measured via a surface plasma resonance or kinetic exclusion assay technique; or
b) has a half-life in human serum of at least 3 days.

In some embodiments, the antigen-binding protein has an on-rate (ka) for GIPR of at least 10⁴/M×seconds, at least 10⁵/M×seconds, or at least 10⁶/M×seconds as measured, for instance, as described below.

In some embodiments, the antigen-binding protein has a slow dissociation rate or off-rate. For example, in some embodiments, the GIPR antigen-binding protein has a kd (off-rate) of 1×10⁻² s⁻¹, or 1×10⁻³ s⁻¹, or 1×10⁻⁴ s⁻¹, or 1×10⁻⁵ s⁻¹.

In some embodiments, the antigen-binding protein has a KD (equilibrium binding affinity) for human GIPR of less than 25 pM, less than 50 pM, less than 100 pM, less than 500 pM, less than 1 nM, less than 5 nM, less than 10 nM, less than 25 nM, or less than 50 nM. In some embodiments, the antigen-binding protein has a KD for human GIPR of 1 nM, 2 nM, 3 nM, 4 nM, 5 nM, 6 nM, 7 nM, 8 nM, 9 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 110 nM, 120 nM, 130 nM, 140 nM, 150 nM, 160 nM, 170 nM, 180 nM, 190 nM, 200 nM, 210 nM, 220 nM, 230 nM, 240 nM, 250 nM, 260 nM, 270 nM, 280 nM, 290 nM, 300 nM, 310 nM, 320 nM, 330 nM, 340 nM, 350 nM, 360 nM, 370 nM, 380 nM, 390 nM, 400 nM, 410 nM, 420 nM, 430 nM, 440 nM, 450 nM, 460 nM, 470 nM, 480 nM, 490 nM, or 500 nM.

In some embodiments, the antigen-binding protein is a polypeptide into which one or more complementary determining regions (CDRs), as described herein, are embedded and/or joined. In some embodiments, the CDRs are embedded into a “framework” region, which orients the CDR(s) such that the proper antigen binding properties of the CDR(s) are achieved.

In some embodiments, the antigen-binding protein is an antibody. In other embodiments, the CDR sequences are embedded in a different type of protein scaffold. Various example protein scaffolds are further described below.

In some embodiments, the antigen-binding protein comprises one or more CDRs (e.g., 1, 2, 3, 4, 5, or 6) as described herein. In some embodiments, the antigen-binding protein comprises (a) a polypeptide structure and (b) one or more CDRs that are inserted into and/or joined to the polypeptide structure. The polypeptide structure can take a variety of different forms. For example, it can be, or comprise, the framework of a naturally occurring antibody, or a fragment or a variant thereof, or may be completely synthetic in nature.

In some embodiments, the polypeptide structure of the antigen-binding protein is an antibody or is derived from an antibody. Non-limiting examples of antigen binding proteins within the scope of this disclosure include, but are not limited to, monoclonal antibodies, bispecific antibodies, minibodies, domain antibodies such as Nanobodies®, synthetic antibodies (sometimes referred to herein as “antibody mimetics”), chimeric antibodies, humanized antibodies, human antibodies, antibody fusions, and portions or fragments of each, respectively. In some embodiments, the antigen-binding protein is an immunological fragment of a complete antibody (e.g., a Fab, a Fab′, a F(ab′)2). In other embodiments, the antigen-binding protein is a scFv that uses CDRs from an anti-GIPR antibody described herein.

In some embodiments, the antigen-binding protein is an antibody that specifically binds to a protein having an amino acid sequence having at least 90% sequence identity to an amino acid sequence of a human GIPR. In some embodiments, the human GIPR has a sequence comprising a sequence selected from the group consisting of SEQ ID NO: 1577, SEQ ID NO: 1578, and SEQ ID NO: 1579.

In some embodiments, the antigen-binding protein is a monoclonal antibody, a recombinant antibody, a human antibody, a humanized antibody, a chimeric antibody, or a multispecific antibody. In some embodiments, the antigen-binding protein is a monoclonal antibody. In some embodiments, the antigen-binding protein is a recombinant antibody. In some embodiments, the antigen-binding protein is a human antibody. In some embodiments, the antigen-binding protein is a humanized antibody. In some embodiments, the antigen-binding protein is a chimeric antibody. In some embodiments, the antigen-binding protein is a multispecific antibody.

In some embodiments, the antigen-binding protein is an antibody is of the IgG1-, IgG2- IgG3- or IgG4-type. In some embodiments, the antibody is of the IgG1-, IgG2-, or IgG4-subclass. In some embodiments, the antibody is of the IgG1- or IgG2-subclass. In some embodiments, the antibody is of the IgG1-type. In some embodiments, the antibody is of the IgG2-type. In some embodiments, the antibody is of the IgG3-type. In some embodiments, the antibody is of the IgG4-type.

In some embodiments, the antigen-binding protein is an antibody inhibits binding of GIP to the extracellular portion of human GIPR.

In some embodiments, the antigen-binding protein (e.g., the anti-GIPR antibody) is conjugated, i.e., covalently bound, directly to an amino acid residue of the polypeptide agonist, or optionally, to a peptidyl or non-peptidyl linker moiety (including, but not limited to, aromatic or aryl linkers) that is covalently bound to an amino acid residue of the polypeptide agonist. For example, in some embodiments, an ε-amino group of a lysine residue of the polypeptide is covalently linked to a C-terminus of a linker polypeptide; and an N-terminus of the linker polypeptide is conjugated to a cysteine residue of the antigen-binding protein (e.g., the anti-GIPR antibody).

Illustratively, in some embodiments, a lysine residue of a polypeptide agonist is covalently linked to a C-terminus of a linker polypeptide via an amide bond, e.g., an amide bond formed by condensation of an ε-amino group of a lysine residue and a carboxyl group of a C-terminus of a polypeptide linker. Additionally, in some embodiments, the N-terminus of the linker polypeptide is derivatized. For example, in some embodiments, the N-terminus of the linker polypeptide is acetylated, such as when a thioether linkage connects an acetylated N-terminus of the linker polypeptide and the cysteine residue of the antigen-binding protein (e.g., the anti-GIPR antibody), wherein the thioether linkage comprises a sulfur atom of the cysteine residue.

In some embodiments, a lysine residue of a polypeptide agonist is covalently linked to a C-terminus of a linker polypeptide via an amide bond, and an acetylated N-terminus of the linker polypeptide is covalently linked to a cysteine residue of the antigen-binding protein (e.g., the anti-GIPR antibody) via a thioether linkage that comprises a sulfur atom of the cysteine residue.

In some embodiments, a lysine residue of a polypeptide agonist is covalently linked to a C-terminus of a linker polypeptide via an amide bond formed by condensation of an ε-amino group of the lysine residue and a carboxyl group of a C-terminus of the polypeptide linker, and an acetylated N-terminus of the linker polypeptide is covalently linked to a cysteine residue of the antigen-binding protein (e.g., the anti-GIPR antibody) via a thioether linkage that comprises a sulfur atom of the cysteine residue.

In some embodiments, the linker polypeptide is a linear polypeptide.

In some embodiments, the linker polypeptide comprises the amino acid sequence of any one of SEQ ID NOs: 1628-1683. In some embodiments, the linker polypeptide comprises the amino acid sequence of any one of SEQ ID NOs: 1628-1630. In some embodiments, the linker polypeptide comprises the amino acid sequence of SEQ ID NO: 1628. In some embodiments, the linker polypeptide comprises the amino acid sequence of SEQ ID NO: 1629. In some embodiments, the linker polypeptide comprises the amino acid sequence of SEQ ID NO: 1630.

Accordingly, in some embodiments, the anti-GIPR antigen-binding protein comprises at least one conjugation site. In some embodiments, the conjugation site is amenable to conjugation of an additional functional moiety (e.g., a glucagon receptor agonist) by a defined conjugation chemistry through the side chain of an amino acid residue at the conjugation site. Achieving highly selective, site-specific conjugation requires consideration of a diverse variety of design criteria. First, a preferred conjugation or coupling chemistry must be defined or predetermined. Functional moieties such as, e.g., glucagon receptor agonists, can be conjugated or coupled to the selected conjugation site of the anti-GIPR antigen-binding protein through an assortment of different conjugation chemistries known in the art. For example, in some embodiments, a maleimide-activated conjugation partner targeting an accessible cysteine thiol on the anti-GIPR antigen-binding protein can be used. In other embodiments, conjugation or coupling chemistries targeting the side chains of either canonical or non-canonical, e.g., unnatural, amino acids in the anti-GIPR antigen binding protein sequence can be used.

Chemistries for chemoselective conjugation of the antigen-binding protein to the polypeptide agonist include, but are not limited to, copper(I)-catalyzed azide-alkyne [3+2] dipolar cycloadditions, Staudinger ligation, other acyl transfers processes, oximations, hydrazone bonding formation, and other suitable organic chemistry reactions such as cross-couplings using water-soluble palladium catalysts. (E.g., Bong et al., Chemoselective Pd(0)-catalyzed peptide coupling in water, Organic Letters 3(16):2509-11 (2001); Dibowski et al., Bioconjugation of peptides by palladium-catalyzed C—C cross-coupling in water, Angew. Chem. Int. Ed. 37(4):476-78 (1998); DeVasher et al., Aqueous-phase, palladium-catalyzed cross-coupling of aryl bromides under mild conditions, using water-soluble, sterically demanding alkylphosphines, J. Org. Chem. 69:7919-27 (2004); Shaugnessy et al., J. Org. Chem, 2003, 68, 6767-6774; Prescher, JA and Bertozzi C R, Chemistry in living system, Nature Chemical Biology 1(1); 13-21 (2005)).

In some embodiments, conjugation (or covalent binding) to the anti-GIPR antigen-binding protein is through the side chain of an amino acid residue at the conjugation site, for example, but not limited to, a cysteinyl residue. The amino acid residue, for example, a cysteinyl residue, at the internal conjugation site that is selected can be one that occupies the same amino acid residue position in a native Fc domain sequence, or the amino acid residue can be engineered into the Fc domain sequence by substitution or insertion.

Non-limiting examples of unnatural amino acid residues that can be useful as a conjugation site include: azido-containing amino acid residues, e.g., azidohomoalanine, p-azido-phenylalanine; keto-containing amino acid residues, e.g., p-acetyl-phenylalanine; alkyne-containing amino acid residues, e.g., p-ethynylphenylalanine, homopropargylglycine, p-(prop-2-ynyl)-tyrosine; alkene-containing amino acid residues e.g., homoallylglycine; aryl halide-containing amino acid residues e.g. p-iodophenylalanine, p-bromophenylalanine; and 1,2-aminothiol containing amino acid residues.

Non-canonical amino acid residues can be incorporated into an anti-GIPR antigen-binding protein by amino acid substitution or insertion. Non-canonical amino acid residues can be incorporated into the peptide by chemical peptide synthesis rather than by synthesis in biological systems, such as recombinantly expressing cells, or alternatively the skilled artisan can employ known techniques of protein engineering that use recombinantly expressing cells. (See, e.g., Link et al., Non-canonical amino acids in protein engineering, Current Opinion in Biotechnology, 14(6):603-609 (2003); Schultz et al., In vivo incorporation of unnatural amino acids, U.S. Pat. No. 7,045,337.)

The selection of the placement of the conjugation site in the overall anti-GIPR antigen binding protein is another relevant facet of selecting an internal conjugation site. Any of the exposed amino acid residues on the anti-GIPR antigen binding protein can be potentially useful conjugation sites and can be mutated to cysteine or some other reactive amino acid for site-selective coupling, if not already present at the selected conjugation site of the anti-GIPR antigen-binding protein sequence. However, this approach does not take into account potential steric constraints that may perturb the activity of the conjugated partner or limit the reactivity of the engineered mutation.

In some embodiments, the anti-GIPR antigen-binding protein is an antibody comprising a cysteine amino acid at one or more conjugation site(s). In some embodiments, the one or more conjugation site(s) is located within the CL, CH1, CH2 or CH3 region of the antibody. In some embodiments, the one or more conjugation site(s) are at positions independently selected from the group consisting of 88 (e.g., D88) of the light chain, 384 (e.g., E384) of the heavy chain, and 487 (e.g., T487) of the heavy chain, according to AHo numbering. For sake of clarity, “D70 of the antibody light chain relative to reference sequence SEQ ID NO: 455” is the same substitution site as AHo position D88 of the light chain of antibody 5G12.006 and Kabat position D70 of the light chain of antibody 5G12.006; “E276 of the antibody heavy chain relative to reference sequence SEQ ID NO: 612” is the same substitution site as AHo position E384 of the heavy chain of antibody 5G12.006 and Kabat position E285 of the heavy chain of antibody 5G12.006; and “T363 of the antibody heavy chain relative to reference sequence SEQ ID NO: 612” is the same substitution site as AHo position T487 of the heavy chain of antibody 5G12.006 and Kabat position T382 of the heavy chain of antibody 5G12.006.

In some embodiments, the anti-GIPR antibody specifically binds to a protein having an amino acid sequence having at least 90% sequence identity to an amino acid sequence of a human GIPR, wherein the antibody comprises at least one cysteine amino acid conjugation site. In some embodiments, the at least one cysteine amino acid conjugation site is selected from the group consisting of 88 of the light chain, 384 of the heavy chain, and 487 of the heavy chain, all according to AHo numbering. In some embodiments, the human GIPR has an amino acid sequence of SEQ ID NO: 1577, SEQ ID NO: 1578, or SEQ ID NO: 1579.

Non-limiting examples of anti-GIPR antibodies are summarized in Table 9. In some embodiments, the antigen-binding protein is an antibody with the CDR, variable domain, and light and heavy chain sequences as specified in one of the rows of Table 9.

SEQ ID NOs have been assigned to variable light chain, variable heavy chain, light chain, heavy chain, CDRL1, CDRL2, CDRL3, CDRH1, CDRH2, and CDRH3 sequences of the non-limiting example antibodies and are shown in Tables 9-17. These antibodies can be identified by SEQ ID NO, but also by construct name (e.g., 2C2.005) or identifier number (e.g., iPS:336175). The anti-GIPR antibodies identified in Tables 9-17 below can be grouped into families based on construct name. For example, the “4B1 family” includes the constructs 4B1, 4B1.010, 4B1.011, 4B1.012, 4B1.013, 4B1.014, 4B1.015, and 4B1.016.

The various light chain and heavy chain variable regions provided herein are depicted in Tables 10 and 11, respectively. Each of these variable regions may be attached to a heavy or light chain constant regions to form a complete antibody heavy and light chain, respectively. Furthermore, each of the so generated heavy and light chain sequences may be combined to form a complete antibody structure.

TABLE 9
Amino Acid SEQ ID NOs. of Example Anti-GIPR Antibodies

| Identifier | Construct | VL | VH | LC | HC | CDRL1 | CDRL2 | CDRL3 | CDRH1 | CDRH2 | CDRH3 |
|---|---|---|---|---|---|---|---|---|---|---|---|
| iPS:336175 | 2C2.005 | 1 | 158 | 315 | 472 | 629 | 786 | 943 | 1100 | 1257 | 1414 |
| iPS:335914 | 4B1 | 2 | 159 | 316 | 473 | 630 | 787 | 944 | 1101 | 1258 | 1415 |
| iPS:335938 | 6H1 | 3 | 160 | 317 | 474 | 631 | 788 | 945 | 1102 | 1259 | 1416 |
| iPS:335941 | 2F11 | 4 | 161 | 318 | 475 | 632 | 789 | 946 | 1103 | 1260 | 1417 |
| iPS:335970 | 5C2 | 5 | 162 | 319 | 476 | 633 | 790 | 947 | 1104 | 1261 | 1418 |
| iPS:335978 | 13H12 | 6 | 163 | 320 | 477 | 634 | 791 | 948 | 1105 | 1262 | 1419 |
| iPS:335986 | 11C1 | 7 | 164 | 321 | 478 | 635 | 792 | 949 | 1106 | 1263 | 1420 |
| iPS:335994 | 12H11 | 8 | 165 | 322 | 479 | 636 | 793 | 950 | 1107 | 1264 | 1421 |
| iPS:336024 | 18E3 | 9 | 166 | 323 | 480 | 637 | 794 | 951 | 1108 | 1265 | 1422 |
| iPS:336041 | 2G10_LC1 | 10 | 167 | 324 | 481 | 638 | 795 | 952 | 1109 | 1266 | 1423 |
| iPS:336077 | 4H9.004 | 11 | 168 | 325 | 482 | 639 | 796 | 953 | 1110 | 1267 | 1424 |
| iPS:336088 | 6A5.004 | 12 | 169 | 326 | 483 | 640 | 797 | 954 | 1111 | 1268 | 1425 |
| iPS:336099 | 17H11.004 | 13 | 170 | 327 | 484 | 641 | 798 | 955 | 1112 | 1269 | 1426 |
| iPS:359621 | 17H11.004.001 | 14 | 171 | 328 | 485 | 642 | 799 | 956 | 1113 | 1270 | 1427 |
| iPS:359781 | 4H9.004.001 | 15 | 172 | 329 | 486 | 643 | 800 | 957 | 1114 | 1271 | 1428 |
| iPS:360929 | 4H9.004.002 | 16 | 173 | 330 | 487 | 644 | 801 | 958 | 1115 | 1272 | 1429 |
| iPS:360936 | 4H9.004.003 | 17 | 174 | 331 | 488 | 645 | 802 | 959 | 1116 | 1273 | 1430 |
| iPS:360943 | 4H9.004.004 | 18 | 175 | 332 | 489 | 646 | 803 | 960 | 1117 | 1274 | 1431 |
| iPS:360949 | 4H9.004.005 | 19 | 176 | 333 | 490 | 647 | 804 | 961 | 1118 | 1275 | 1432 |
| iPS:359761 | 4H9.004.006 | 20 | 177 | 334 | 491 | 648 | 805 | 962 | 1119 | 1276 | 1433 |
| iPS:360955 | 4B1.010 | 21 | 178 | 335 | 492 | 649 | 806 | 963 | 1120 | 1277 | 1434 |
| iPS:360962 | 4B1.011 | 22 | 179 | 336 | 493 | 650 | 807 | 964 | 1121 | 1278 | 1435 |
| iPS:360968 | 4B1.012 | 23 | 180 | 337 | 494 | 651 | 808 | 965 | 1122 | 1279 | 1436 |
| iPS:360974 | 4B1.013 | 24 | 181 | 338 | 495 | 652 | 809 | 966 | 1123 | 1280 | 1437 |
| iPS:360978 | 4B1.014 | 25 | 182 | 339 | 496 | 653 | 810 | 967 | 1124 | 1281 | 1438 |
| iPS:359785 | 4B1.015 | 26 | 183 | 340 | 497 | 654 | 811 | 968 | 1125 | 1282 | 1439 |
| iPS:360982 | 4B1.016 | 27 | 184 | 341 | 498 | 655 | 812 | 969 | 1126 | 1283 | 1440 |
| iPS:335922 | 18F2 | 28 | 185 | 342 | 499 | 656 | 813 | 970 | 1127 | 1284 | 1441 |
| iPS:360986 | 18F2.002 | 29 | 186 | 343 | 500 | 657 | 814 | 971 | 1128 | 1285 | 1442 |
| iPS:360995 | 18F2.003 | 30 | 187 | 344 | 501 | 658 | 815 | 972 | 1129 | 1286 | 1443 |
| iPS:360999 | 18F2.004 | 31 | 188 | 345 | 502 | 659 | 816 | 973 | 1130 | 1287 | 1444 |
| iPS:361005 | 18F2.005 | 32 | 189 | 346 | 503 | 660 | 817 | 974 | 1131 | 1288 | 1445 |
| iPS:361009 | 18F2.006 | 33 | 190 | 347 | 504 | 661 | 818 | 975 | 1132 | 1289 | 1446 |
| iPS:361013 | 18F2.007 | 34 | 191 | 348 | 505 | 662 | 819 | 976 | 1133 | 1290 | 1447 |
| iPS:361017 | 18F2.008 | 35 | 192 | 349 | 506 | 663 | 820 | 977 | 1134 | 1291 | 1448 |
| iPS:361021 | 18F2.009 | 36 | 193 | 350 | 507 | 664 | 821 | 978 | 1135 | 1292 | 1449 |
| iPS:361028 | 18F2.010 | 37 | 194 | 351 | 508 | 665 | 822 | 979 | 1136 | 1293 | 1450 |
| iPS:360535 | 18F2.011 | 38 | 195 | 352 | 509 | 666 | 823 | 980 | 1137 | 1294 | 1451 |
| iPS:361035 | 18F2.012 | 39 | 196 | 353 | 510 | 667 | 824 | 981 | 1138 | 1295 | 1452 |
| iPS:359940 | 2F11.002 | 40 | 197 | 354 | 511 | 668 | 825 | 982 | 1139 | 1296 | 1453 |
| iPS:361039 | 2F11.003 | 41 | 198 | 355 | 512 | 669 | 826 | 983 | 1140 | 1297 | 1454 |
| iPS:361043 | 2F11.004 | 42 | 199 | 356 | 513 | 670 | 827 | 984 | 1141 | 1298 | 1455 |
| iPS:361049 | 2F11.005 | 43 | 200 | 357 | 514 | 671 | 828 | 985 | 1142 | 1299 | 1456 |
| iPS:361055 | 2F11.006 | 44 | 201 | 358 | 515 | 672 | 829 | 986 | 1143 | 1300 | 1457 |
| iPS:361059 | 2F11.007 | 45 | 202 | 359 | 516 | 673 | 830 | 987 | 1144 | 1301 | 1458 |
| iPS:361063 | 2F11.008 | 46 | 203 | 360 | 517 | 674 | 831 | 988 | 1145 | 1302 | 1459 |
| iPS:359949 | 2F11.009 | 47 | 204 | 361 | 518 | 675 | 832 | 989 | 1146 | 1303 | 1460 |
| iPS:359956 | 2F11.010 | 48 | 205 | 362 | 519 | 676 | 833 | 990 | 1147 | 1304 | 1461 |
| iPS:359865 | 6H1.002 | 49 | 206 | 363 | 520 | 677 | 834 | 991 | 1148 | 1305 | 1462 |
| iPS:359869 | 6H1.003 | 50 | 207 | 364 | 521 | 678 | 835 | 992 | 1149 | 1306 | 1463 |
| iPS:359873 | 6H1.004 | 51 | 208 | 365 | 522 | 679 | 836 | 993 | 1150 | 1307 | 1464 |
| iPS:359877 | 6H1.005 | 52 | 209 | 366 | 523 | 680 | 837 | 994 | 1151 | 1308 | 1465 |
| iPS:361067 | 6H1.006 | 53 | 210 | 367 | 524 | 681 | 838 | 995 | 1152 | 1309 | 1466 |
| iPS:361071 | 6H1.007 | 54 | 211 | 368 | 525 | 682 | 839 | 996 | 1153 | 1310 | 1467 |
| iPS:361075 | 6H1.008 | 55 | 212 | 369 | 526 | 683 | 840 | 997 | 1154 | 1311 | 1468 |
| iPS:361079 | 6A5.004.001 | 56 | 213 | 370 | 527 | 684 | 841 | 998 | 1155 | 1312 | 1469 |
| iPS:361085 | 6A5.004.002 | 57 | 214 | 371 | 528 | 685 | 842 | 999 | 1156 | 1313 | 1470 |
| iPS:361091 | 6A5.004.003 | 58 | 215 | 372 | 529 | 686 | 843 | 1000 | 1157 | 1314 | 1471 |
| iPS:361095 | 6A5.004.004 | 59 | 216 | 373 | 530 | 687 | 844 | 1001 | 1158 | 1315 | 1472 |
| iPS:361101 | 6A5.004.005 | 60 | 217 | 374 | 531 | 688 | 845 | 1002 | 1159 | 1316 | 1473 |
| iPS:361105 | 6A5.004.006 | 61 | 218 | 375 | 532 | 689 | 846 | 1003 | 1160 | 1317 | 1474 |
| iPS:361109 | 6A5.004.007 | 62 | 219 | 376 | 533 | 690 | 847 | 1004 | 1161 | 1318 | 1475 |
| iPS:361113 | 6A5.004.008 | 63 | 220 | 377 | 534 | 691 | 848 | 1005 | 1162 | 1319 | 1476 |
| iPS:361120 | 6A5.004.009 | 64 | 221 | 378 | 535 | 692 | 849 | 1006 | 1163 | 1320 | 1477 |
| iPS:359896 | 6A5.004.010 | 65 | 222 | 379 | 536 | 693 | 850 | 1007 | 1164 | 1321 | 1478 |
| iPS:359890 | 6A5.004.011 | 66 | 223 | 380 | 537 | 694 | 851 | 1008 | 1165 | 1322 | 1479 |
| iPS:359567 | 2A11.002 | 67 | 224 | 381 | 538 | 695 | 852 | 1009 | 1166 | 1323 | 1480 |
| iPS:335965 | 2A11.003 | 68 | 225 | 382 | 539 | 696 | 853 | 1010 | 1167 | 1324 | 1481 |
| iPS:361158 | 2A11.004 | 69 | 226 | 383 | 540 | 697 | 854 | 1011 | 1168 | 1325 | 1482 |
| iPS:361165 | 2A11.005 | 70 | 227 | 384 | 541 | 698 | 855 | 1012 | 1169 | 1326 | 1483 |
| iPS:361172 | 2G10_LC1.003 | 71 | 228 | 385 | 542 | 699 | 856 | 1013 | 1170 | 1327 | 1484 |
| iPS:361178 | 2G10_LC1.004 | 72 | 229 | 386 | 543 | 700 | 857 | 1014 | 1171 | 1328 | 1485 |
| iPS:361185 | 2G10_LC1.005 | 73 | 230 | 387 | 544 | 701 | 858 | 1015 | 1172 | 1329 | 1486 |
| iPS:361192 | 2G10_LC1.006 | 74 | 231 | 388 | 545 | 702 | 859 | 1016 | 1173 | 1330 | 1487 |
| iPS:359609 | 2G10_LC1.007 | 75 | 232 | 389 | 546 | 703 | 860 | 1017 | 1174 | 1331 | 1488 |
| iPS:359615 | 2G10_LC1.008 | 76 | 233 | 390 | 547 | 704 | 861 | 1018 | 1175 | 1332 | 1489 |
| iPS:361196 | 2G10_LC1.009 | 77 | 234 | 391 | 548 | 705 | 862 | 1019 | 1176 | 1333 | 1490 |
| iPS:361202 | 2G10_LC1.010 | 78 | 235 | 392 | 549 | 706 | 863 | 1020 | 1177 | 1334 | 1491 |
| iPS:359644 | 18E3.002 | 79 | 236 | 393 | 550 | 707 | 864 | 1021 | 1178 | 1335 | 1492 |
| PS:361206 | 18E3.003 | 80 | 237 | 394 | 551 | 708 | 865 | 1022 | 1179 | 1336 | 1493 |
| iPS:359628 | 18E3.004 | 81 | 238 | 395 | 552 | 709 | 866 | 1023 | 1180 | 1337 | 1494 |
| iPS:359637 | 18E3.005 | 82 | 239 | 396 | 553 | 710 | 867 | 1024 | 1181 | 1338 | 1495 |
| iPS:361210 | 18E3.006 | 83 | 240 | 397 | 554 | 711 | 868 | 1025 | 1182 | 1339 | 1496 |
| iPS:361214 | 18E3.007 | 84 | 241 | 398 | 555 | 712 | 869 | 1026 | 1183 | 1340 | 1497 |
| iPS:361218 | 18E3.008 | 85 | 242 | 399 | 556 | 713 | 870 | 1027 | 1184 | 1341 | 1498 |
| iPS:361222 | 5C2.006 | 86 | 243 | 400 | 557 | 714 | 871 | 1028 | 1185 | 1342 | 1499 |
| iPS:361229 | 5C2.007 | 87 | 244 | 401 | 558 | 715 | 872 | 1029 | 1186 | 1343 | 1500 |
| iPS:361236 | 5C2.008 | 88 | 245 | 402 | 559 | 716 | 873 | 1030 | 1187 | 1344 | 1501 |
| iPS:359685 | 5C2.009 | 89 | 246 | 403 | 560 | 717 | 874 | 1031 | 1188 | 1345 | 1502 |
| iPS:361240 | 5C2.010 | 90 | 247 | 404 | 561 | 718 | 875 | 1032 | 1189 | 1346 | 1503 |
| iPS:361247 | 5C2.011 | 91 | 248 | 405 | 562 | 719 | 876 | 1033 | 1190 | 1347 | 1504 |
| iPS:361254 | 5C2.012 | 92 | 249 | 406 | 563 | 720 | 877 | 1034 | 1191 | 1348 | 1505 |
| iPS:361261 | 5C2.013 | 93 | 250 | 407 | 564 | 721 | 878 | 1035 | 1192 | 1349 | 1506 |
| iPS:361268 | 5C2.014 | 94 | 251 | 408 | 565 | 722 | 879 | 1036 | 1193 | 1350 | 1507 |
| iPS:359669 | 5C2.015 | 95 | 252 | 409 | 566 | 723 | 880 | 1037 | 1194 | 1351 | 1508 |
| iPS:359678 | 5C2.016 | 96 | 253 | 410 | 567 | 724 | 881 | 1038 | 1195 | 1352 | 1509 |
| iPS:361275 | 11C1.002 | 97 | 254 | 411 | 568 | 725 | 882 | 1039 | 1196 | 1353 | 1510 |
| iPS:361282 | 11C1.003 | 98 | 255 | 412 | 569 | 726 | 883 | 1040 | 1197 | 1354 | 1511 |
| iPS:361289 | 11C1.004 | 99 | 256 | 413 | 570 | 727 | 884 | 1041 | 1198 | 1355 | 1512 |
| iPS:359712 | 11C1.005 | 100 | 257 | 414 | 571 | 728 | 885 | 1042 | 1199 | 1356 | 1513 |
| iPS:361293 | 11C1.006 | 101 | 258 | 415 | 572 | 729 | 886 | 1043 | 1200 | 1357 | 1514 |
| iPS:361297 | 11C1.007 | 102 | 259 | 416 | 573 | 730 | 887 | 1044 | 1201 | 1358 | 1515 |
| iPS:361301 | 11C1.008 | 103 | 260 | 417 | 574 | 731 | 888 | 1045 | 1202 | 1359 | 1516 |
| iPS:359703 | 11C1.009 | 104 | 261 | 418 | 575 | 732 | 889 | 1046 | 1203 | 1360 | 1517 |
| iPS:361305 | 11C1.010 | 105 | 262 | 419 | 576 | 733 | 890 | 1047 | 1204 | 1361 | 1518 |
| iPS:361312 | 13H12.002 | 106 | 263 | 420 | 577 | 734 | 891 | 1048 | 1205 | 1362 | 1519 |
| iPS:359744 | 13H12.003 | 107 | 264 | 421 | 578 | 735 | 892 | 1049 | 1206 | 1363 | 1520 |
| iPS:361319 | 13H12.004 | 108 | 265 | 422 | 579 | 736 | 893 | 1050 | 1207 | 1364 | 1521 |
| iPS:359737 | 13H12.005 | 109 | 266 | 423 | 580 | 737 | 894 | 1051 | 1208 | 1365 | 1522 |
| iPS:361326 | 13H12.006 | 110 | 267 | 424 | 581 | 738 | 895 | 1052 | 1209 | 1366 | 1523 |
| iPS:361330 | 12H11.002 | 111 | 268 | 425 | 582 | 739 | 896 | 1053 | 1210 | 1367 | 1524 |
| iPS:361337 | 12H11.003 | 112 | 269 | 426 | 583 | 740 | 897 | 1054 | 1211 | 1368 | 1525 |
| iPS:361344 | 12H11.004 | 113 | 270 | 427 | 584 | 741 | 898 | 1055 | 1212 | 1369 | 1526 |
| iPS:361351 | 12H11.005 | 114 | 271 | 428 | 585 | 742 | 899 | 1056 | 1213 | 1370 | 1527 |
| iPS:361358 | 12H11.006 | 115 | 272 | 429 | 586 | 743 | 900 | 1057 | 1214 | 1371 | 1528 |
| iPS:361365 | 12H11.007 | 116 | 273 | 430 | 587 | 744 | 901 | 1058 | 1215 | 1372 | 1529 |
| iPS:361372 | 12H11.008 | 117 | 274 | 431 | 588 | 745 | 902 | 1059 | 1216 | 1373 | 1530 |
| iPS:361379 | 12H11.009 | 118 | 275 | 432 | 589 | 746 | 903 | 1060 | 1217 | 1374 | 1531 |
| iPS:361383 | 12H11.010 | 119 | 276 | 433 | 590 | 747 | 904 | 1061 | 1218 | 1375 | 1532 |
| iPS:361387 | 12H11.011 | 120 | 277 | 434 | 591 | 748 | 905 | 1062 | 1219 | 1376 | 1533 |
| iPS:361391 | 12H11.012 | 121 | 278 | 435 | 592 | 749 | 906 | 1063 | 1220 | 1377 | 1534 |
| iPS:361395 | 12H11.013 | 122 | 279 | 436 | 593 | 750 | 907 | 1064 | 1221 | 1378 | 1535 |
| iPS:361402 | 12H11.014 | 123 | 280 | 437 | 594 | 751 | 908 | 1065 | 1222 | 1379 | 1536 |
| iPS:361406 | 2C2.005.001 | 124 | 281 | 438 | 595 | 752 | 909 | 1066 | 1223 | 1380 | 1537 |
| iPS:361412 | 2C2.005.002 | 125 | 282 | 439 | 596 | 753 | 910 | 1067 | 1224 | 1381 | 1538 |
| iPS:361418 | 2C2.005.003 | 126 | 283 | 440 | 59 | 754 | 911 | 1068 | 1225 | 1382 | 1539 |
| iPS:361424 | 2C2.005.004 | 127 | 284 | 441 | 598 | 755 | 912 | 1069 | 1226 | 1383 | 1540 |
| iPS:361430 | 2C2.005.005 | 128 | 285 | 442 | 599 | 756 | 913 | 1070 | 1227 | 1384 | 1541 |
| iPS:361436 | 2C2.005.006 | 129 | 286 | 443 | 600 | 757 | 914 | 1071 | 1228 | 1385 | 1542 |
| iPS:361442 | 2C2.005.007 | 130 | 287 | 444 | 601 | 758 | 915 | 1072 | 1229 | 1386 | 1543 |
| iPS:361448 | 2C2.005.008 | 131 | 288 | 445 | 602 | 759 | 916 | 1073 | 1230 | 1387 | 1544 |
| iPS:361454 | 2C2.005.009 | 132 | 289 | 446 | 603 | 760 | 917 | 1074 | 1231 | 1388 | 1545 |
| iPS:361460 | 2C2.005.010 | 133 | 290 | 447 | 604 | 761 | 918 | 1075 | 1232 | 1389 | 1546 |
| iPS:361466 | 2C2.005.011 | 134 | 291 | 448 | 605 | 762 | 919 | 1076 | 1233 | 1390 | 1547 |
| iPS:361472 | 2C2.005.012 | 135 | 292 | 449 | 606 | 763 | 920 | 1077 | 1234 | 1391 | 1548 |
| iPS:361478 | 2C2.005.013 | 136 | 293 | 450 | 607 | 764 | 921 | 1078 | 1235 | 1392 | 1549 |
| iPS:361485 | 2C2.005.014 | 137 | 294 | 451 | 608 | 765 | 922 | 1079 | 1236 | 1393 | 1550 |
| iPS:361845 | 18F2.013 | 138 | 295 | 452 | 609 | 766 | 923 | 1080 | 1237 | 1394 | 1551 |
| iPS:361851 | 6H1.009 | 139 | 296 | 453 | 610 | 767 | 924 | 1081 | 1238 | 1395 | 1552 |
| iPS:361855 | 2C2.005.015 | 140 | 297 | 454 | 611 | 768 | 925 | 1082 | 1239 | 1396 | 1553 |
| iPS:336067 | 5G12.006 | 141 | 298 | 455 | 612 | 769 | 926 | 1083 | 1240 | 1397 | 1554 |
| iPS:336169 | 17B11.002 | 142 | 299 | 456 | 613 | 770 | 927 | 1084 | 1241 | 1398 | 1555 |
| iPS:361127 | 5G12.006.001 | 143 | 300 | 457 | 614 | 771 | 928 | 1085 | 1242 | 1399 | 1556 |
| iPS:361136 | 5G12.006.002 | 144 | 301 | 458 | 615 | 772 | 929 | 1086 | 1243 | 1400 | 1557 |
| iPS:361140 | 5G12.006.003 | 145 | 302 | 459 | 616 | 773 | 930 | 1087 | 1244 | 1401 | 1558 |
| iPS:359919 | 5G12.006.004 | 146 | 303 | 460 | 617 | 774 | 931 | 1088 | 1245 | 1402 | 1559 |
| iPS:361144 | 5G12.006.005 | 147 | 304 | 461 | 618 | 775 | 932 | 1089 | 1246 | 1403 | 1560 |
| iPS:361151 | 5G12.006.006 | 148 | 305 | 462 | 619 | 776 | 933 | 1090 | 1247 | 1404 | 1561 |
| iPS:361491 | 17B11.002.001 | 149 | 306 | 463 | 620 | 777 | 934 | 1091 | 1248 | 1405 | 1562 |
| iPS:361499 | 17B11.002.002 | 150 | 307 | 464 | 621 | 778 | 935 | 1092 | 1249 | 1406 | 1563 |
| iPS:361503 | 17B11.002.003 | 151 | 308 | 465 | 622 | 779 | 936 | 1093 | 1250 | 1407 | 1564 |
| iPS:361507 | 17B11.002.004 | 152 | 309 | 466 | 623 | 780 | 937 | 1094 | 1251 | 1408 | 1565 |
| iPS:361514 | 17B11.002.005 | 153 | 310 | 467 | 624 | 781 | 938 | 1095 | 1252 | 1409 | 1566 |
| iPS:360582 | 17B11.002.006 | 154 | 311 | 468 | 625 | 782 | 939 | 1096 | 1253 | 1410 | 1567 |
| iPS:361518 | 17B11.002.007 | 155 | 312 | 469 | 626 | 783 | 940 | 1097 | 1254 | 1411 | 1568 |
| iPS:360570 | 17B11.002.008 | 156 | 313 | 470 | 627 | 784 | 941 | 1098 | 1255 | 1412 | 1569 |
| iPS:362051 | 5G12.005.001 | 157 | 314 | 471 | 628 | 785 | 942 | 1099 | 1256 | 1413 | 1570 |

TABLE 10
Variable Light (VL) Region Amino Acid Sequences of Example Anti-GIPR Antibodies

| | | | SEQ |
|---|---|---|---|
| Identifier | Construct | VL | ID NO: |
| iPS:336175 | 2C2.005 | QSVLTQPPSASGTPGQRVTISCSGSSSNIGSNTVNWYQHLPGTAPKLLIYTNNQRPS | 1 |
| | | GVPDRFSGSKSGTSASLAISGLQSEDEADYFCATFDDSLNGPVFGGGTKLTVLG | |
| iPS:335914 | 4B1 | DIQMTQSPSSLSASIGDRVTITCRASQDIRDYLGWYQQKPGKAPKRLIYGASSLQS | 2 |
| | | GVPSRFSGSGSGTEFTLTISSLQPEDFATYYCLQHNNYPFTFGPGTKVDIKR | |
| iPS:335938 | 6H1 | DIQMTQSPSSLSASIGDRVTITCRASQDIRDYLGWYQQKPGKAPKRLIYGASSLQS | 3 |
| | | GVPSRFSGSGSGTEFTLTISSLQPEDFATYYCLQHNNYPFTFGPGTKVDIKR | |
| iPS:335941 | 2F11 | DIQMTQSPSSLSASVGDRVTITCQASQDISNYLNWYQQKPGKAPKLLIYDASNLET | 4 |
| | | GVPSRFSGSGSGTDFSFTISSLQPEDIATYYCQQYDILLTFGGGTKVEIKR | |
| iPS:335970 | 5C2 | DIVMTQSPDSLAVSLGERATINCKSSQSVLSSSNNKNYIAWYQQQPGHPPKLLIYW | 5 |
| | | ASTRESGVPDRFSGSGSGTDFTLTISSLQAEDVAVFYCQQYYSTPWTFGQGTKVEI | |
| | | KR | |
| iPS:335978 | 13H12 | DIVMTQSPDSLAVSLGERATINCKSSQSVLSSSNNKNYIAWYQQKPGHPPKLLIYW | 6 |
| | | ASTRESGVPDRFSGSGSGTDFTLTISSLQAEDVAVFYCQQYYSTPWTFGQGTKVEI | |
| | | KR | |
| iPS:335986 | 11C1 | DIVMTQSPDSLSVSLGERATINCKSSQSVLSSSNNKNYLAWYQQKPGQPPNLLIY | 7 |
| | | WTSTRDSGVPDRFSGSGSGTDFTLTINSLQAEDVAVYYCQQYYRTPWTFGQGTK | |
| | | VEIKR | |
| iPS:335994 | 12H11 | DIVMTQSPDSLSVSLGERATINCKSSQSVLSSSNNKNYLAWYQQKPGQPPNLLIY | 8 |
| | | WTSTRDSGVPDRFSGSGSGTDFTLTINSLQAEDVAVYYCQQYYRTPWTFGQGTK | |
| | | VEIKR | |
| iPS:336024 | 18E3 | EIVLTQSPGTLSLSPGERATLSCRASQSISYSYLAWYQQKPGQAPRLLIYGASSRAT | 9 |
| | | GIPDRFSGSGSGTDFTLTISRQEPDDFAVYYCQQYGSSPLTFGGGTKVEIKR | |
| iPS:336041 | 2G10_LC1 | EIVMTQSPATLSVSPGERATLSCRASQSVSSNLAWYQQKPGQAPRLLIYGAATRA | 10 |
| | | TGIPARVSGSGSGTEFTLTISSLQSEDFAVYYCQQYNNWPLTFGGGTKVEIKR | |
| iPS:336077 | 4H9.004 | DIQMTQSPSSLSASVGDRVTITCRASQGIRNELGWYQQKPGKAPKRLIYGASSLQS | 11 |
| | | GVPSRFSGSGSGTEFTLTISSLQPEDFVTYYCLQHNSYPFTFGPGTKVDVKR | |
| iPS:336088 | 6A5.004 | DIQMTQSPSSLSASVGDRVTITCQASQDITNYLNWYQQKPGKAPKLLIYDASNLEP | 12 |
| | | GVPSRFSGSGSGTDFTFTISSLQPEDIATYYCQQYDDLFTFGPGTKVDIKR | |
| iPS:336099 | 17H11.004 | DIQMTQSPSSVSASVGDRVTITCRASQGLIIWLAWYQQKPGKAPKLLIYAASSLQS | 13 |
| | | GVPSRFSGSGSGTDFTLTISSLQPEDFATYYCQQTNSFPPTFGQGTKVEIKR | |
| iPS:359621 | 17H11.004.001 | DIQMTQSPSSVSASVGDRVTITCRASQGLIIWLAWYQQKPGKAPKLLIYAASSLQS | 14 |
| | | GVPSRFSGSGSGTDFTLTISSLQPEDFATYYCQQTNSFPPTFGQGTKVEIKR | |
| iPS:359781 | 4H9.004.001 | DIQMTQSPSSLSASVGDRVTITCRASQGIRNELGWYQQKPGKAPKLLIYGASSLQS | 15 |
| | | GVPSRFSGSGSGTEFTLTISSLQPEDFVTYYCLQHNSYPFTFGQGTKVDVKR | |
| iPS:360929 | 4H9.004.002 | DIQMTQSPSSLSASVGDRVTITCRASQGIRNELGWYQQKPGKAPKLLIYGASSLQS | 16 |
| | | GVPSRFSGSGSGTEFTLTISSLQPEDFVTYYCLQHNSYPFTFGQGTKVDVKR | |
| iPS:360936 | 4H9.004.003 | DIQMTQSPSSLSASVGDRVTITCRASQGIRNELGWYQQKPGKAPKLLIYGASSLQS | 17 |
| | | GVPSRFSGSGSGTEFTLTISSLQPEDFVTYYCLQHNSYPFTFGQGTKVDVKR | |
| iPS:360943 | 4H9.004.004 | DIQMTQSPSSLSASVGDRVTITCRASQGIRNELGWYQQKPGKAPKLLIYGASSLQS | 18 |
| | | GVPSRFSGSGSGTEFTLTISSLQPEDFVTYYCLQHNSYPFTFGPGTKVDVKR | |
| iPS:360949 | 4H9.004.005 | DIQMTQSPSSLSASVGDRVTITCRASQGIRNELGWYQQKPGKAPKRLIYGASSLQS | 19 |
| | | GVPSRFSGSGSGTEFTLTISSLQPEDFVTYYCLQHNSYPFTFGQGTKVDVKR | |
| iPS:359761 | 4H9.004.006 | DIQMTQSPSSLSASVGDRVTITCRASQGIRNELGWYQQKPGKAPKLLIYGASSLQS | 20 |
| | | GVPSRFSGSGSGTEFTLTISSLQPEDFVTYYCLQHNSYPFTFGQGTKVDVKR | |
| iPS:360955 | 4B1.010 | DIQMTQSPSSLSASIGDRVTITCRASQDIRDYLGWYQQKPGKAPKLLIYGASSLQS | 21 |
| | | GVPSRFSGSGSGTEFTLTISSLQPEDFATYYCLQHNNYPFTFGQGTKVDIKR | |
| iPS:360962 | 4B1.011 | DIQMTQSPSSLSASIGDRVTITCRASQDIRDYLGWYQQKPGKAPKRLIYGASSLQS | 22 |
| | | GVPSRFSGSGSGTEFTLTISSLQPEDFATYYCLQHNNYPFTFGQGTKVDIKR | |
| iPS:360968 | 4B1.012 | DIQMTQSPSSLSASIGDRVTITCRASQDIRDYLGWYQQKPGKAPKLLIYGASSLQS | 23 |
| | | GVPSRFSGSGSGTEFTLTISSLQPEDFATYYCLQHNNYPFTFGPGTKVDIKR | |
| iPS:360974 | 4B1.013 | DIQMTQSPSSLSASIGDRVTITCRASQDIRDYLGWYQQKPGKAPKLLIYGASSLQS | 24 |
| | | GVPSRFSGSGSGTEFTLTISSLQPEDFATYYCLQHNNYPFTFGQGTKVDIKR | |
| iPS:360978 | 4B1.014 | DIQMTQSPSSLSASIGDRVTITCRASQDIRDYLGWYQQKPGKAPKLLIYGASSLQS | 25 |
| | | GVPSRFSGSGSGTEFTLTISSLQPEDFATYYCLQHNNYPFTFGQGTKVDIKR | |
| iPS:359785 | 4B1.015 | DIQMTQSPSSLSASIGDRVTITCRASQDIRDYLGWYQQKPGKAPKLLIYGASSLQS | 26 |
| | | GVPSRFSGSGSGTEFTLTISSLQPEDFATYYCLQHNNYPFTFGQGTKVDIKR | |
| iPS:360982 | 4B1.016 | DIQMTQSPSSLSASIGDRVTITCRASQDIRDYLGWYQQKPGKAPKLLIYGASSLQS | 27 |
| | | GVPSRFSGSGSGTEFTLTISSLQPEDFATYYCLQHNNYPFTFGQGTKVDIKR | |
| iPS:335922 | 18F2 | DIQMTQSPSSLSASIGDRVTITCRASQDLRNYLGWYHQKPGKAPKRLIYGASSLQS | 28 |
| | | GVPSRFSGSGSGTEFTLTISSLQPEDFATYYCLQHNNYPFTFGPGTKVDIKR | |
| iPS:360986 | 18F2.002 | DIQMTQSPSSLSASIGDRVTITCRASQDLRNYLGWYQQKPGKAPKLLIYGASSLQS | 29 |
| | | GVPSRFSGSGSGTEFTLTISSLQPEDFATYYCLQHNNYPFTFGPGTKVDIKR | |
| iPS:360995 | 18F2.003 | DIQMTQSPSSLSASIGDRVTITCRASQDLRNYLGWYQQKPGKAPKLLIYGASSLQS | 30 |
| | | GVPSRFSGSGSGTEFTLTISSLQPEDFATYYCLQHNNYPFTFGPGTKVDIKR | |
| iPS:360999 | 18F2.004 | DIQMTQSPSSLSASIGDRVTITCRASQDLRNYLGWYHQKPGKAPKLLIYGASSLQS | 31 |
| | | GVPSRFSGSGSGTEFTLTISSLQPEDFATYYCLQHNNYPFTFGPGTKVDIKR | |
| iPS:361005 | 18F2.005 | DIQMTQSPSSLSASIGDRVTITCRASQDLRNYLGWYQQKPGKAPKRLIYGASSLQS | 32 |
| | | GVPSRFSGSGSGTEFTLTISSLQPEDFATYYCLQHNNYPFTFGPGTKVDIKR | |
| iPS:361009 | 18F2.006 | DIQMTQSPSSLSASIGDRVTITCRASQDLRNYLGWYQQKPGKAPKRLIYGASSLQS | 33 |
| | | GVPSRFSGSGSGTEFTLTISSLQPEDFATYYCLQHNNYPFTFGPGTKVDIKR | |
| iPS:361013 | 18F2.007 | DIQMTQSPSSLSASIGDRVTITCRASQDLRNYLGWYHQKPGKAPKLLIYGASSLQS | 34 |
| | | GVPSRFSGSGSGTEFTLTISSLQPEDFATYYCLQHNNYPFTFGPGTKVDIKR | |
| iPS:361017 | 18F2.008 | DIQMTQSPSSLSASIGDRVTITCRASQDLRNYLGWYHQKPGKAPKRLIYGASSLQS | 35 |
| | | GVPSRFSGSGSGTEFTLTISSLQPEDFATYYCLQHNNYPFTFGPGTKVDIKR | |
| iPS:361021 | 18F2.009 | DIQMTQSPSSLSASIGDRVTITCRASQDLRNYLGWYQQKPGKAPKLLIYGASSLQS | 36 |
| | | GVPSRFSGSGSGTEFTLTISSLQPEDFATYYCLQHNNYPFTFGPGTKVDIKR | |
| iPS:361028 | 18F2.010 | DIQMTQSPSSLSASIGDRVTITCRASQDLRNYLGWYQQKPGKAPKLLIYGASSLQS | 37 |
| | | GVPSRFSGSGSGTEFTLTISSLQPEDFATYYCLQHNNYPFTFGPGTKVDIKR | |
| iPS:360535 | 18F2.011 | DIQMTQSPSSLSASIGDRVTITCRASQDLRNYLGWYQQKPGKAPKLLIYGASSLQS | 38 |
| | | GVPSRFSGSGSGTEFTLTISSLQPEDFATYYCLQHNNYPFTFGQGTKVDIKR | |
| iPS:361035 | 18F2.012 | DIQMTQSPSSLSASIGDRVTITCRASQDLRNYLGWYQQKPGKAPKLLIYGASSLQS | 39 |
| | | GVPSRFSGSGSGTEFTLTISSLQPEDFATYYCLQHNNYPFTFGPGTKVDIKR | |
| iPS:359940 | 2F11.002 | DIQMTQSPSSLSASVGDRVTITCQASQDISNYLNWYQQKPGKAPKLLIYDASNLET | 40 |
| | | GVPSRFSGSGSGTDFSLTISSLQPEDFATYYCQQYDILLTFGGGTKVEIKR | |
| iPS:361039 | 2F11.003 | DIQMTQSPSSLSASVGDRVTITCQASQDISNYLNWYQQKPGKAPKLLIYDASNLET | 41 |
| | | GVPSRFSGSGSGTDFSLTISSLQPEDFATYYCQQYDILLTFGGGTKVEIKR | |
| iPS:361043 | 2F11.004 | DIQMTQSPSSLSASVGDRVTITCQASQDISNYLNWYQQKPGKAPKLLIYDASNLET | 42 |
| | | GVPSRFSGSGSGTDFSLTISSLQPEDIATYYCQQYDILLTFGGGTKVEIKR | |
| iPS:361049 | 2F11.005 | DIQMTQSPSSLSASVGDRVTITCQASQDISNYLNWYQQKPGKAPKLLIYDASNLET | 43 |
| | | GVPSRFSGSGSGTDFSFTISSLQPEDFATYYCQQYDILLTFGGGTKVEIKR | |
| iPS:361055 | 2F11.006 | DIQMTQSPSSLSASVGDRVTITCQASQDISNYLNWYQQKPGKAPKLLIYDASNLET | 44 |
| | | GVPSRFSGSGSGTDFSLTISSLQPEDIATYYCQQYDILLTFGGGTKVEIKR | |
| iPS:361059 | 2F11.007 | DIQMTQSPSSLSASVGDRVTITCQASQDISNYLNWYQQKPGKAPKLLIYDASNLET | 45 |
| | | GVPSRFSGSGSGTDFSFTISSLQPEDFATYYCQQYDILLTFGGGTKVEIKR | |
| iPS:361063 | 2F11.008 | DIQMTQSPSSLSASVGDRVTITCQASQDISNYLNWYQQKPGKAPKLLIYDASNLET | 46 |
| | | GVPSRFSGSGSGTDFSFTISSLQPEDIATYYCQQYDILLTFGGGTKVEIKR | |
| iPS:359949 | 2F11.009 | DIQMTQSPSSLSASVGDRVTITCQASQDISNYLNWYQQKPGKAPKLLIYDASNLET | 47 |
| | | GVPSRFSGSGSGTDFSLTISSLQPEDFATYYCQQYDILLTFGGGTKVEIKR | |
| iPS:359956 | 2F11.010 | DIQMTQSPSSLSASVGDRVTITCQASQDISNYLNWYQQKPGKAPKLLIYDASNLET | 48 |
| | | GVPSRFSGSGSGTDFSLTISSLQPEDFATYYCQQYDILLTFGGGTKVEIKR | |
| iPS:359865 | 6H1.002 | DIQMTQSPSSLSASIGDRVTITCRASQDIRDYLGWYQQKPGKAPKRLIYGASSLQS | 49 |
| | | GVPSRFSGSGSGTEFTLTISSLQPEDFATYYCLQHNNYPFTFGPGTKVDIKR | |
| iPS:359869 | 6H1.003 | DIQMTQSPSSLSASIGDRVTITCRASQDIRDYLGWYQQKPGKAPKRLIYGASSLQS | 50 |
| | | GVPSRFSGSGSGTEFTLTISSLQPEDFATYYCLQHNNYPFTFGPGTKVDIKR | |
| iPS:359873 | 6H1.004 | DIQMTQSPSSLSASIGDRVTITCRASQDIRDYLGWYQQKPGKAPKLLIYGASSLQS | 51 |
| | | GVPSRFSGSGSGTEFTLTISSLQPEDFATYYCLQHNNYPFTFGQGTKVDIKR | |
| iPS:359877 | 6H1.005 | DIQMTQSPSSLSASIGDRVTITCRASQDIRDYLGWYQQKPGKAPKLLIYGASSLQS | 52 |
| | | GVPSRFSGSGSGTEFTLTISSLQPEDFATYYCLQHNNYPFTFGQGTKVDIKR | |
| iPS:361067 | 6H1.006 | DIQMTQSPSSLSASIGDRVTITCRASQDIRDYLGWYQQKPGKAPKLLIYGASSLQS | 53 |
| | | GVPSRFSGSGSGTEFTLTISSLQPEDFATYYCLQHNNYPFTFGPGTKVDIKR | |
| iPS:361071 | 6H1.007 | DIQMTQSPSSLSASIGDRVTITCRASQDIRDYLGWYQQKPGKAPKLLIYGASSLQS | 54 |
| | | GVPSRFSGSGSGTEFTLTISSLQPEDFATYYCLQHNNYPFTFGPGTKVDIKR | |
| iPS:361075 | 6H1.008 | DIQMTQSPSSLSASIGDRVTITCRASQDIRDYLGWYQQKPGKAPKLLIYGASSLQS | 55 |
| | | GVPSRFSGSGSGTEFTLTISSLQPEDFATYYCLQHNNYPFTFGPGTKVDIKR | |
| iPS:361079 | 6A5.004.001 | DIQMTQSPSSLSASVGDRVTITCQASQDITNYLNWYQQKPGKAPKLLIYDASNLEP | 56 |
| | | GVPSRFSGSGSGTDFTLTISSLQPEDFATYYCQQYDDLFTFGPGTKVDIKR | |
| iPS:361085 | 6A5.004.002 | DIQMTQSPSSLSASVGDRVTITCQASQDITNYLNWYQQKPGKAPKLLIYDASNLEP | 57 |
| | | GVPSRFSGSGSGTDFTLTISSLQPEDIATYYCQQYDDLFTFGPGTKVDIKR | |
| iPS:361091 | 6A5.004.003 | DIQMTQSPSSLSASVGDRVTITCQASQDITNYLNWYQQKPGKAPKLLIYDASNLEP | 58 |
| | | GVPSRFSGSGSGTDFTLTISSLQPEDFATYYCQQYDDLFTFGPGTKVDIKR | |
| iPS:361095 | 6A5.004.004 | DIQMTQSPSSLSASVGDRVTITCQASQDITNYLNWYQQKPGKAPKLLIYDASNLEP | 59 |
| | | GVPSRFSGSGSGTDFTFTISSLQPEDFATYYCQQYDDLFTFGPGTKVDIKR | |
| iPS:361101 | 6A5.004.005 | DIQMTQSPSSLSASVGDRVTITCQASQDITNYLNWYQQKPGKAPKLLIYDASNLEP | 60 |
| | | GVPSRFSGSGSGTDFTLTISSLQPEDIATYYCQQYDDLFTFGPGTKVDIKR | |
| iPS:361105 | 6A5.004.006 | DIQMTQSPSSLSASVGDRVTITCQASQDITNYLNWYQQKPGKAPKLLIYDASNLEP | 61 |
| | | GVPSRFSGSGSGTDFTFTISSLQPEDFATYYCQQYDDLFTFGPGTKVDIKR | |
| iPS:361109 | 6A5.004.007 | DIQMTQSPSSLSASVGDRVTITCQASQDITNYLNWYQQKPGKAPKLLIYDASNLEP | 62 |
| | | GVPSRFSGSGSGTDFTFTISSLQPEDIATYYCQQYDDLFTFGPGTKVDIKR | |
| iPS:361113 | 6A5.004.008 | DIQMTQSPSSLSASVGDRVTITCQASQDITNYLNWYQQKPGKAPKLLIYDASNLEP | 63 |
| | | GVPSRFSGSGSGTDFTLTISSLQPEDFATYYCQQYDDLFTFGPGTKVDIKR | |
| iPS:361120 | 6A5.004.009 | DIQMTQSPSSLSASVGDRVTITCQASQDITNYLNWYQQKPGKAPKLLIYDASNLEP | 64 |
| | | GVPSRFSGSGSGTDFTLTISSLQPEDFATYYCQQYDDLFTFGPGTKVDIKR | |
| iPS:359896 | 6A5.004.010 | DIQMTQSPSSLSASVGDRVTITCQASQDITNYLNWYQQKPGKAPKLLIYDASNLEP | 65 |
| | | GVPSRFSGSGSGTDFTLTISSLQPEDFATYYCQQYDDLFTFGQGTKVDIKR | |
| iPS:359890 | 6A5.004.011 | DIQMTQSPSSLSASVGDRVTITCQASQDITNYLNWYQQKPGKAPKLLIYDASNLEP | 66 |
| | | GVPSRFSGSGSGTDFTLTISSLQPEDFATYYCQQYDDLFTFGQGTKVDIKR | |
| iPS:359567 | 2A11.002 | EIVLTQSPGTLSLSPGERATLSCRASQIFTSTYLAWYQQKPGQAPRLLIYGASSRAT | 67 |
| | | GIPDRFSGSGSGTDFTLTISRLEPEDFAVYYCQQYGSSPRFGQGTRLEIKR | |
| iPS:335965 | 2A11.003 | EIVLTQSPGTLSLSPGERATLSCRASQIFTSTYLAWYQQKPGQAPRLLIYGASSRAT | 68 |
| | | GIPDRFSGSGSGTDFTLTISRLEPEDFAVYYCQQYGSSPRFGQGTRLEIKR | |
| iPS:361158 | 2A11.004 | EIVLTQSPGTLSLSPGERATLSCRASQIFTSTYLAWYQQKPGQAPRLLIYGASSRAT | 69 |
| | | GIPDRFSGSGSGTDFTLTISRLEPEDFAVYYCQQYGSSPRFGQGTRLEIKR | |
| iPS:361165 | 2A11.005 | EIVLTQSPGTLSLSPGERATLSCRASQIFTSTYLAWYQQKPGQAPRLLIYGASSRAT | 70 |
| | | GIPDRFSGSGSGTDFTLTISRLEPEDFAVYYCQQYGSSPRFGQGTRLEIKR | |
| iPS:361172 | 2G10_LC1.003 | EIVMTQSPATLSVSPGERATLSCRASQSVSSNLAWYQQKPGQAPRLLIYGAATRA | 71 |
| | | TGIPARFSGSGSGTEFTLTISSLQSEDFAVYYCQQYNNWPLTFGGGTKVEIKR | |
| iPS:361178 | 2G10_LC1.004 | EIVMTQSPATLSVSPGERATLSCRASQSVSSNLAWYQQKPGQAPRLLIYGAATRA | 72 |
| | | TGIPARFSGSGSGTEFTLTISSLQSEDFAVYYCQQYNNWPLTFGGGTKVEIKR | |
| iPS:361185 | 2G10_LC1.005 | EIVMTQSPATLSVSPGERATLSCRASQSVSSNLAWYQQKPGQAPRLLIYGAATRA | 73 |
| | | TGIPARFSGSGSGTEFTLTISSLQSEDFAVYYCQQYNNWPLTFGGGTKVEIKR | |
| iPS:361192 | 2G10_LC1.006 | EIVMTQSPATLSVSPGERATLSCRASQSVSSNLAWYQQKPGQAPRLLIYGAATRA | 74 |
| | | TGIPARVSGSGSGTEFTLTISSLQSEDFAVYYCQQYNNWPLTFGGGTKVEIKR | |
| iPS:359609 | 2G10_LC1.007 | EIVMTQSPATLSVSPGERATLSCRASQSVSSNLAWYQQKPGQAPRLLIYGAATRA | 75 |
| | | TGIPARFSGSGSGTEFTLTISSLEPEDFAVYYCQQYNNFPLTFGGGTKVEIKR | |
| iPS:359615 | 2G10_LC1.008 | EIVMTQSPATLSVSPGERATLSCRASQSVSSNLAWYQQKPGQAPRLLIYGAATRA | 76 |
| | | TGIPARFSGSGSGTEFTLTISSLEPEDFAVYYCQQYNNYPLTFGGGTKVEIKR | |
| iPS:361196 | 2G10_LC1.009 | EIVMTQSPATLSVSPGERATLSCRASQSVSSNLAWYQQKPGQAPRLLIYGAATRA | 77 |
| | | TGIPARFSGSGSGTEFTLTISSLEPEDFAVYYCQQYNNWPLTFGGGTKVEIKR | |
| iPS:361202 | 2G10_LC1.010 | EIVMTQSPATLSVSPGERATLSCRASQSVSSNLAWYQQKPGQAPRLLIYGAATRA | 78 |
| | | TGIPARFSGSGSGTEFTLTISSLEPEDFAVYYCQQYNNWPLTFGGGTKVEIKR | |
| iPS:359644 | 18E3.002 | EIVLTQSPGTLSLSPGERATLSCRASQSISYSYLAWYQQKPGQAPRLLIYGASSRAT | 79 |
| | | GIPDRFSGSGSGTDFTLTISRLEPDDFAVYYCQQYGSSPLTFGGGTKVEIKR | |
| iPS:361206 | 18E3.003 | EIVLTQSPGTLSLSPGERATLSCRASQSISYSYLAWYQQKPGQAPRLLIYGASSRAT | 80 |
| | | GIPDRFSGSGSGTDFTLTISRQEPDDFAVYYCQQYGSSPLTFGGGTKVEIKR | |
| iPS:359628 | 18E3.004 | EIVLTQSPGTLSLSPGERATLSCRASQSISYSYLAWYQQKPGQAPRLLIYGASSRAT | 81 |
| | | GIPDRFSGSGSGTDFTLTISRLEPDDFAVYYCQQYGSSPLTFGGGTKVEIKR | |
| iPS:359637 | 18E3.005 | EIVLTQSPGTLSLSPGERATLSCRASQSISYSYLAWYQQKPGQAPRLLIYGASSRAT | 82 |
| | | GIPDRFSGSGSGTDFTLTISRLEPDDFAVYYCQQYGSSPLTFGGGTKVEIKR | |
| iPS:361210 | 18E3.006 | EIVLTQSPGTLSLSPGERATLSCRASQSISYSYLAWYQQKPGQAPRLLIYGASSRAT | 83 |
| | | GIPDRFSGSGSGTDFTLTISRLEPDDFAVYYCQQYGSSPLTFGGGTKVEIKR | |
| iPS:361214 | 18E3.007 | EIVLTQSPGTLSLSPGERATLSCRASQSISYSYLAWYQQKPGQAPRLLIYGASSRAT | 84 |
| | | GIPDRFSGSGSGTDFTLTISRQEPDDFAVYYCQQYGSSPLTFGGGTKVEIKR | |
| iPS:361218 | 18E3.008 | EIVLTQSPGTLSLSPGERATLSCRASQSISYSYLAWYQQKPGQAPRLLIYGASSRAT | 85 |
| | | GIPDRFSGSGSGTDFTLTISRQEPDDFAVYYCQQYGSSPLTFGGGTKVEIKR | |
| iPS:361222 | 5C2.006 | DIVMTQSPDSLAVSLGERATINCKSSQSVLSSSNNKNYIAWYQQQPGHPPKLLIYW | 86 |
| | | ASTRESGVPDRFSGSGSGTDFTLTISSLQAEDVAVFYCQQYYSTPWTFGQGTKVEI | |
| | | KR | |
| iPS:361229 | 5C2.007 | DIVMTQSPDSLAVSLGERATINCKSSQSVLSSSNNKNYIAWYQQQPGHPPKLLIYW | 87 |
| | | ASTRESGVPDRFSGSGSGTDFTLTISSLQAEDVAVFYCQQYYSTPWTFGQGTKVEI | |
| | | KR | |
| iPS:361236 | 5C2.008 | DIVMTQSPDSLAVSLGERATINCKSSQSVLSSSNNKNYIAWYQQQPGHPPKLLIYW | 88 |
| | | ASTRESGVPDRFSGSGSGTDFTLTISSLQAEDVAVFYCQQYYSTPWTFGQGTKVEI | |
| | | KR | |
| iPS:359685 | 5C2.009 | DIVMTQSPDSLAVSLGERATINCKSSQSVLSSSNNKNYIAWYQQQPGHPPKLLIYW | 89 |
| | | ASTRESGVPDRFSGSGSGTDFTLTISSLQAEDVAVFYCQQYYSTPWTFGQGTKVEI | |
| | | KR | |
| iPS:361240 | 5C2.010 | DIVMTQSPDSLAVSLGERATINCKSSQSVLSSSNNKNYIAWYQQQPGHPPKLLIYW | 90 |
| | | ASTRESGVPDRFSGSGSGTDFTLTISSLQAEDVAVFYCQQYYSTPWTFGQGTKVEI | |
| | | KR | |
| iPS:361247 | 5C2.011 | DIVMTQSPDSLAVSLGERATINCKSSQSVLSSSNNKNYIAWYQQQPGHPPKLLIYW | 91 |
| | | ASTRESGVPDRFSGSGSGTDFTLTISSLQAEDVAVFYCQQYYSTPWTFGQGTKVEI | |
| | | KR | |
| iPS:361254 | 5C2.012 | DIVMTQSPDSLAVSLGERATINCKSSQSVLSSSNNKNYIAWYQQQPGHPPKLLIYW | 92 |
| | | ASTRESGVPDRFSGSGSGTDFTLTISSLQAEDVAVFYCQQYYSTPWTFGQGTKVEI | |
| | | KR | |
| iPS:361261 | 5C2.013 | DIVMTQSPDSLAVSLGERATINCKSSQSVLSSSNNKNYIAWYQQQPGHPPKLLIYW | 93 |
| | | ASTRESGVPDRFSGSGSGTDFTLTISSLQAEDVAVFYCQQYYSTPWTFGQGTKVEI | |
| | | KR | |
| iPS:361268 | 5C2.014 | DIVMTQSPDSLAVSLGERATINCKSSQSVLSSSNNKNYIAWYQQQPGHPPKLLIYW | 94 |
| | | ASTRESGVPDRFSGSGSGTDFTLTISSLQAEDVAVFYCQQYYSTPWTFGQGTKVEI | |
| | | KR | |
| iPS:359669 | 5C2.015 | DIVMTQSPDSLAVSLGERATINCKSSQSVLSSSNNKNYIAWYQQKPGQPPKLLIYW | 95 |
| | | ASTRESGVPDRFSGSGSGTDFTLTISSLQAEDVAVFYCQQYYSTPWTFGQGTKVEI | |
| | | KR | |
| iPS:359678 | 5C2.016 | DIVMTQSPDSLAVSLGERATINCKSSQSVLSSSNNKNYIAWYQQKPGQPPKLLIYW | 96 |
| | | ASTRESGVPDRFSGSGSGTDFTLTISSLQAEDVAVFYCQQYYSTPWTFGQGTKVEI | |
| | | KR | |
| iPS:361275 | 11C1.002 | DIVMTQSPDSLSVSLGERATINCKSSQSVLSSSNNKNYLAWYQQKPGQPPNLLIY | 97 |
| | | WTSTRESGVPDRFSGSGSGTDFTLTINSLQAEDVAVYYCQQYYRTPWTFGQGTKV | |
| | | EIKR | |
| iPS:361282 | 11C1.003 | DIVMTQSPDSLSVSLGERATINCKSSQSVLSSSNNKNYLAWYQQKPGQPPNLLIY | 98 |
| | | WTSTRESGVPDRFSGSGSGTDFTLTINSLQAEDVAVYYCQQYYRTPWTFGQGTKV | |
| | | EIKR | |
| iPS:361289 | 11C1.004 | DIVMTQSPDSLSVSLGERATINCKSSQSVLSSSNNKNYLAWYQQKPGQPPNLLIY | 99 |
| | | WTSTRDSGVPDRFSGSGSGTDFTLTINSLQAEDVAVYYCQQYYRTPWTFGQGTK | |
| | | VEIKR | |
| iPS:359712 | 11C1.005 | DIVMTQSPDSLSVSLGERATINCKSSQSVLSSSNNKNYLAWYQQKPGQPPNLLIY | 100 |
| | | WTSTRESGVPDRFSGSGSGTDFTLTINSLQAEDVAVYYCQQYYRTPWTFGQGTKV | |
| | | EIKR | |
| iPS:361293 | 11C1.006 | DIVMTQSPDSLSVSLGERATINCKSSQSVLSSSNNKNYLAWYQQKPGQPPNLLIY | 101 |
| | | WTSTRESGVPDRFSGSGSGTDFTLTINSLQAEDVAVYYCQQYYRTPWTFGQGTKV | |
| | | EIKR | |
| iPS:361297 | 11C1.007 | DIVMTQSPDSLSVSLGERATINCKSSQSVLSSSNNKNYLAWYQQKPGQPPNLLIY | 102 |
| | | WTSTRDSGVPDRFSGSGSGTDFTLTINSLQAEDVAVYYCQQYYRTPWTFGQGTK | |
| | | VEIKR | |
| iPS:361301 | 11C1.008 | DIVMTQSPDSLSVSLGERATINCKSSQSVLSSSNNKNYLAWYQQKPGQPPNLLIY | 103 |
| | | WTSTRDSGVPDRFSGSGSGTDFTLTINSLQAEDVAVYYCQQYYRTPWTFGQGTK | |
| | | VEIKR | |
| iPS:359703 | 11C1.009 | DIVMTQSPDSLAVSLGERATINCKSSQSVLSSSNNKNYLAWYQQKPGQPPKLLIY | 104 |
| | | WTSTRESGVPDRFSGSGSGTDFTLTINSLQAEDVAVYYCQQYYRTPWTFGQGTKV | |
| | | EIKR | |
| iPS:361305 | 11C1.010 | DIVMTQSPDSLSVSLGERATINCKSSQSVLSSSNNKNYLAWYQQKPGQPPNLLIY | 105 |
| | | WTSTRESGVPDRFSGSGSGTDFTLTINSLQAEDVAVYYCQQYYRTPWTFGQGTKV | |
| | | EIKR | |
| iPS:361312 | 13H12.002 | DIVMTQSPDSLAVSLGERATINCKSSQSVLSSSNNKNYIAWYQQKPGHPPKLLIYW | 106 |
| | | ASTRESGVPDRFSGSGSGTDFTLTISSLQAEDVAVFYCQQYYSTPWTFGQGTKVEI | |
| | | KR | |
| iPS:359744 | 13H12.003 | DIVMTQSPDSLAVSLGERATINCKSSQSVLSSSNNKNYIAWYQQKPGHPPKLLIYW | 107 |
| | | ASTRESGVPDRFSGSGSGTDFTLTISSLQAEDVAVFYCQQYYSTPWTFGQGTKVEI | |
| | | KR | |
| iPS:361319 | 13H12.004 | DIVMTQSPDSLAVSLGERATINCKSSQSVLSSSNNKNYIAWYQQKPGHPPKLLIYW | 108 |
| | | ASTRESGVPDRFSGSGSGTDFTLTISSLQAEDVAVFYCQQYYSTPWTFGQGTKVEI | |
| | | KR | |
| iPS:359737 | 13H12.005 | DIVMTQSPDSLAVSLGERATINCKSSQSVLSSSNNKNYIAWYQQKPGQPPKLLIYW | 109 |
| | | ASTRESGVPDRFSGSGSGTDFTLTISSLQAEDVAVFYCQQYYSTPWTFGQGTKVEI | |
| | | KR | |
| iPS:361326 | 13H12.006 | DIVMTQSPDSLAVSLGERATINCKSSQSVLSSSNNKNYIAWYQQKPGQPPKLLIYW | 110 |
| | | ASTRESGVPDRFSGSGSGTDFTLTISSLQAEDVAVFYCQQYYSTPWTFGQGTKVEI | |
| | | KR | |
| iPS:361330 | 12H11.002 | DIVMTQSPDSLSVSLGERATINCKSSQSVLSSSNNKNYLAWYQQKPGQPPNLLIY | 111 |
| | | WTSTRDSGVPDRFSGSGSGTDFTLTINSLQAEDVAVYYCQQYYRTPWTFGQGTK | |
| | | VEIKR | |
| iPS:361337 | 12H11.003 | DIVMTQSPDSLSVSLGERATINCKSSQSVLSSSNNKNYLAWYQQKPGQPPNLLIY | 112 |
| | | WTSTRDSGVPDRFSGSGSGTDFTLTINSLQAEDVAVYYCQQYYRTPWTFGQGTK | |
| | | VEIKR | |
| iPS:361344 | 12H11.004 | DIVMTQSPDSLSVSLGERATINCKSSQSVLSSSNNKNYLAWYQQKPGQPPNLLIY | 113 |
| | | WTSTRDSGVPDRFSGSGSGTDFTLTINSLQAEDVAVYYCQQYYRTPWTFGQGTK | |
| | | VEIKR | |
| iPS:361351 | 12H11.005 | DIVMTQSPDSLSVSLGERATINCKSSQSVLSSSNNKNYLAWYQQKPGQPPNLLIY | 114 |
| | | WTSTRDSGVPDRFSGSGSGTDFTLTINSLQAEDVAVYYCQQYYRTPWTFGQGTK | |
| | | VEIKR | |
| iPS:361358 | 12H11.006 | DIVMTQSPDSLSVSLGERATINCKSSQSVLSSSNNKNYLAWYQQKPGQPPNLLIY | 115 |
| | | WTSTRDSGVPDRFSGSGSGTDFTLTINSLQAEDVAVYYCQQYYRTPWTFGQGTK | |
| | | VEIKR | |
| iPS:361365 | 12H11.007 | DIVMTQSPDSLSVSLGERATINCKSSQSVLSSSNNKNYLAWYQQKPGQPPNLLIY | 116 |
| | | WTSTRDSGVPDRFSGSGSGTDFTLTINSLQAEDVAVYYCQQYYRTPWTFGQGTK | |
| | | VEIKR | |
| iPS:361372 | 12H11.008 | DIVMTQSPDSLSVSLGERATINCKSSQSVLSSSNNKNYLAWYQQKPGQPPNLLIY | 117 |
| | | WTSTRDSGVPDRFSGSGSGTDFTLTINSLQAEDVAVYYCQQYYRTPWTFGQGTK | |
| | | VEIKR | |
| iPS:361379 | 12H11.009 | DIVMTQSPDSLSVSLGERATINCKSSQSVLSSSNNKNYLAWYQQKPGQPPNLLIY | 118 |
| | | WTSTRESGVPDRFSGSGSGTDFTLTINSLQAEDVAVYYCQQYYRTPWTFGQGTKV | |
| | | EIKR | |
| iPS:361383 | 12H11.010 | DIVMTQSPDSLSVSLGERATINCKSSQSVLSSSNNKNYLAWYQQKPGQPPNLLIY | 119 |
| | | WTSTRESGVPDRFSGSGSGTDFTLTINSLQAEDVAVYYCQQYYRTPWTFGQGTKV | |
| | | EIKR | |
| iPS:361387 | 12H11.011 | DIVMTQSPDSLSVSLGERATINCKSSQSVLSSSNNKNYLAWYQQKPGQPPNLLIY | 120 |
| | | WTSTRESGVPDRFSGSGSGTDFTLTINSLQAEDVAVYYCQQYYRTPWTFGQGTKV | |
| | | EIKR | |
| iPS:361391 | 12H11.012 | DIVMTQSPDSLSVSLGERATINCKSSQSVLSSSNNKNYLAWYQQKPGQPPNLLIY | 121 |
| | | WTSTRESGVPDRFSGSGSGTDFTLTINSLQAEDVAVYYCQQYYRTPWTFGQGTKV | |
| | | EIKR | |
| iPS:361395 | 12H11.013 | DIVMTQSPDSLAVSLGERATINCKSSQSVLSSSNNKNYLAWYQQKPGQPPKLLIY | 122 |
| | | WTSTRDSGVPDRFSGSGSGTDFTLTINSLQAEDVAVYYCQQYYRTPWTFGQGTK | |
| | | VEIKR | |
| iPS:361402 | 12H11.014 | DIVMTQSPDSLAVSLGERATINCKSSQSVLSSSNNKNYLAWYQQKPGQPPKLLIY | 123 |
| | | WTSTRESGVPDRFSGSGSGTDFTLTINSLQAEDVAVYYCQQYYRTPWTFGQGTKV | |
| | | EIKR | |
| iPS:361406 | 2C2.005.001 | QSVLTQPPSASGTPGQRVTISCSGSSSNIGSQTVNWYQHLPGTAPKLLIYTNNQRPS | 124 |
| | | GVPDRFSGSKSGTSASLAISGLQSEDEADYFCATFDESLSGPVFGGGTKLTVLG | |
| iPS:361412 | 2C2.005.002 | QSVLTQPPSASGTPGQRVTISCSGSSSNIGSQTVNWYQHLPGTAPKLLIYTNNQRPS | 125 |
| | | GVPDRFSGSKSGTSASLAISGLQSEDEADYFCATFDESLQGPVFGGGTKLTVLG | |
| iPS:361418 | 2C2.005.003 | QSVLTQPPSASGTPGQRVTISCSGSSSNIGSNYVNWYQHLPGTAPKLLIYTNNQRPS | 126 |
| | | GVPDRFSGSKSGTSASLAISGLQSEDEADYFCATFDESLSGPVFGGGTKLTVLG | |
| iPS:361424 | 2C2.005.004 | QSVLTQPPSASGTPGQRVTISCSGSSSNIGSNYVNWYQHLPGTAPKLLIYTNNQRPS | 127 |
| | | GVPDRFSGSKSGTSASLAISGLQSEDEADYFCATFDESLQGPVFGGGTKLTVLG | |
| iPS:361430 | 2C2.005.005 | QSVLTQPPSASGTPGQRVTISCSGSSSNIGSQTVNWYQHLPGTAPKLLIYTNNQRPS | 128 |
| | | GVPDRFSGSKSGTSASLAISGLQSEDEADYFCATFDDSLNGPVFGGGTKLTVLG | |
| iPS:361436 | 2C2.005.006 | QSVLTQPPSASGTPGQRVTISCSGSSSNIGSNYVNWYQHLPGTAPKLLIYTNNQRPS | 129 |
| | | GVPDRFSGSKSGTSASLAISGLQSEDEADYFCATFDDSLNGPVFGGGTKLTVLG | |
| iPS:361442 | 2C2.005.007 | QSVLTQPPSASGTPGQRVTISCSGSSSNIGSNTVNWYQHLPGTAPKLLIYTNNQRPS | 130 |
| | | GVPDRFSGSKSGTSASLAISGLQSEDEADYFCATFDESLNGPVFGGGTKLTVLG | |
| iPS:361448 | 2C2.005.008 | QSVLTQPPSASGTPGQRVTISCSGSSSNIGSNTVNWYQHLPGTAPKLLIYTNNQRPS | 131 |
| | | GVPDRFSGSKSGTSASLAISGLQSEDEADYFCATFDDSLQGPVFGGGTKLTVLG | |
| iPS:361454 | 2C2.005.009 | QSVLTQPPSASGTPGQRVTISCSGSSSNIGSNTVNWYQHLPGTAPKLLIYTNNQRPS | 132 |
| | | GVPDRFSGSKSGTSASLAISGLQSEDEADYFCATFDDSLSGPVFGGGTKLTVLG | |
| iPS:361460 | 2C2.005.010 | QSVLTQPPSASGTPGQRVTISCSGSSSNIGSNTVNWYQHLPGTAPKLLIYTNNQRPS | 133 |
| | | GVPDRFSGSKSGTSASLAISGLQSEDEADYFCATFDDSLNAPVFGGGTKLTVLG | |
| iPS:361466 | 2C2.005.011 | QSVLTQPPSASGTPGQRVTISCSGSSSNIGSNTVNWYQHLPGTAPKLLIYTNNQRPS | 134 |
| | | GVPDRFSGSKSGTSASLAISGLQSEDEADYFCATFDSSLNGPVFGGGTKLTVLG | |
| iPS:361472 | 2C2.005.012 | QSVLTQPPSASGTPGQRVTISCSGSSSNIGSNYVNWYQHLPGTAPKLLIYTNNQRPS | 135 |
| | | GVPDRFSGSKSGTSASLAISGLQSEDEADYFCATFDSSLNAPVFGGGTKLTVLG | |
| iPS:361478 | 2C2.005.013 | QSVLTQPPSASGTPGQRVTISCSGSSSNIGSNYVNWYQHLPGTAPKLLIYTNNQRPS | 136 |
| | | GVPDRFSGSKSGTSASLAISGLQSEDEADYFCATFDSSLNAPVFGGGTKLTVLG | |
| iPS:361485 | 2C2.005.014 | QSVLTQPPSASGTPGQRVTISCSGSSSNIGSQTVNWYQHLPGTAPKLLIYTNNQRPS | 137 |
| | | GVPDRFSGSKSGTSASLAISGLQSEDEADYFCATFDESLSGPVFGGGTKLTVLG | |
| iPS:361845 | 18F2.013 | DIQMTQSPSSLSASIGDRVTITCRASQDLRNYLGWYQQKPGKAPKRLIYGASSLQS | 138 |
| | | GVPSRFSGSGSGTEFTLTISSLQPEDFATYYCLQHNNYPFTFGQGTKVDIKR | |
| iPS:361851 | 6H1.009 | DIQMTQSPSSLSASIGDRVTITCRASQDIRDYLGWYQQKPGKAPKRLIYGASSLQS | 139 |
| | | GVPSRFSGSGSGTEFTLTISSLQPEDFATYYCLQHNNYPFTFGQGTKVDIKR | |
| iPS:361855 | 2C2.005.015 | QSVLTQPPSASGTPGQRVTISCSGSSSNIGSNTVNWYQQLPGTAPKLLIYTNNQRPS | 140 |
| | | GVPDRFSGSKSGTSASLAISGLQSEDEADYFCATFDSSLSGPVFGGGTKLTVLG | |
| iPS:336067 | 5G12.006 | DIQLTQSPSSLSASVGDRVTITCRASQTISRFLNWYQQKPGKAPELLIYVASSLQSG | 141 |
| | | VPSRFSGSGSGTDFTLTISSLQPEDFATYYCQQSYSTLISFGQGTKLEITR | |
| iPS:336169 | 17B11.002 | QSVLTQPPSVSAAPGQKVTISCSGSSSNIGNNYVSWYEQFPGTAPKLLIYDNNKRP | 142 |
| | | SGIPDRFSGSKSGTSATLGITGLQTGDEADYYCGTWDSSLSAVVFGGGTKLTVLG | |
| iPS:361127 | 5G12.006.001 | DIQLTQSPSSLSASVGDRVTITCRASQTISRFLNWYQQKPGKAPELLIYVASSLQSG | 143 |
| | | VPSRFSGSGSGTDFTLTISSLQPEDFATYYCQQSYSTLISFGQGTKLEIKR | |
| iPS:361136 | 5G12.006.002 | DIQLTQSPSSLSASVGDRVTITCRASQTISRFLNWYQQKPGKAPELLIYVASSLQSG | 144 |
| | | VPSRFSGSGSGTDFTLTISSLQPEDFATYYCQQSYSTLISFGQGTKLEIKR | |
| iPS:361140 | 5G12.006.003 | DIQLTQSPSSLSASVGDRVTITCRASQTISRFLNWYQQKPGKAPELLIYVASSLQSG | 145 |
| | | VPSRFSGSGSGTDFTLTISSLQPEDFATYYCQQSYSTLISFGQGTKLEITR | |
| iPS:359919 | 5G12.006.004 | DIQLTQSPSSLSASVGDRVTITCRASQTISRFLNWYQQKPGKAPKLLIYVASSLQSG | 146 |
| | | VPSRFSGSGSGTDFTLTISSLQPEDFATYYCQQSYSTLISFGQGTKLEIKR | |
| iPS:361144 | 5G12.006.005 | DIQLTQSPSSLSASVGDRVTITCRASQTISRFLNWYQQKPGKAPELLIYVASSLQSG | 147 |
| | | VPSRFSGSGSGTDFTLTISSLQPEDFATYYCQQSYSTLISFGQGTKLEIKR | |
| iPS:361151 | 5G12.006.006 | DIQLTQSPSSLSASVGDRVTITCRASQTISRFLNWYQQKPGKAPELLIYVASSLQSG | 148 |
| | | VPSRFSGSGSGTDFTLTISSLQPEDFATYYCQQSYSTLISFGQGTKLEIKR | |
| iPS:361491 | 17B11.002.001 | QSVLTQPPSVSAAPGQKVTISCSGSSSNIGNNYVSWYQQLPGTAPKLLIYDNNKRP | 149 |
| | | SGIPDRFSGSKSGTSATLGITGLQTGDEADYYCGTWDSSLSAVVFGGGTKLTVLG | |
| iPS:361499 | 17B11.002.002 | QSVLTQPPSVSAAPGQKVTISCSGSSSNIGNNYVSWYQQLPGTAPKLLIYDNNKRP | 150 |
| | | SGIPDRFSGSKSGTSATLGITGLQTGDEADYYCGTWDSSLSAVVFGGGTKLTVLG | |
| iPS:361503 | 17B11.002.003 | QSVLTQPPSVSAAPGQKVTISCSGSSSNIGNNYVSWYEQFPGTAPKLLIYDNNKRP | 151 |
| | | SGIPDRFSGSKSGTSATLGITGLQTGDEADYYCGTWDSSLSAVVFGGGTKLTVLG | |
| iPS:361507 | 17B11.002.004 | QSVLTQPPSVSAAPGQKVTISCSGSSSNIGNNYVSWYQQLPGTAPKLLIYDNNKRP | 152 |
| | | SGIPDRFSGSKSGTSATLAITGLQTGDEADYYCGTFESSLSAVVFGGGTKLTVLG | |
| iPS:361514 | 17B11.002.005 | QSVLTQPPSVSAAPGQKVTISCSGSSSNIGNNYVSWYQQLPGTAPKLLIYDNNKRP | 153 |
| | | SGIPDRFSGSKSGTSATLAITGLQTGDEADYYCGTWDSSLSAVVFGGGTKLTVLG | |
| iPS:360582 | 17B11.002.006 | QSVLTQPPSVSAAPGQKVTISCSGSSSNIGNNYVSWYQQLPGTAPKLLIYDNNKRP | 154 |
| | | SGIPDRFSGSKSGTSATLAITGLQTGDEADYYCGTWDSSLSAVVFGGGTKLTVLG | |
| iPS:361518 | 17B11.002.007 | QSVLTQPPSVSAAPGQKVTISCSGSSSNIGNNYVSWYQQLPGTAPKLLIYDNNKRP | 155 |
| | | SGIPDRFSGSKSGTSATLGITGLQTGDEADYYCGTWDSSLSAVVFGGGTKLTVLG | |
| iPS:360570 | 17B11.002.008 | QSVLTQPPSVSAAPGQKVTISCSGSSSNIGNNYVSWYQQLPGTAPKLLIYDNNKRP | 156 |
| | | SGIPDRFSGSKSGTSATLAITGLQTGDEADYYCGTFESSLSAVVFGGGTKLTVLG | |
| iPS:362051 | 5G12.005.001 | DIQLTQSPSSLSASVGDRVTITCRASQTISRFLNWYQQKPGKAPKLLIYVASSLQSG | 157 |
| | | VPSRFSGSGSGTDFTLTISSLQPEDFATYYCQQSYSTLLSFGQGTKLEIKR | |

TABLE 11
Variable Heavy (VH) Region Amino Acid Sequences of Example Anti-GIPR Antibodies

| | | | SEQ |
|---|---|---|---|
| | | | ID |
| Identifier | Construct | VH | NO: |
| iPS:336175 | 2C2.005 | QMQVVQSGAEVKKPGASVKVSCKASGYTFTGYYMHWVRQAPGQGLEWMGWIN | 158 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:335914 | 4B1 | QVQLVESGGGVVQPGRSLRLSCAASGFTFSNFGMHWVRQAPGKGLEWVAVIWY | 159 |
| | | DGSNENYADSVKGRFTISRDNSKNTLYLHMNSLRVADTAVYYCARDGTIFGVVL | |
| | | GDYWGQGTLVTVSS | |
| iPS:335938 | 6H1 | QVQLVESGGGVVQPGRSLRLSCAASGFTFSYFGMHWVRQAPGKGLEWVAVIWY | 160 |
| | | DGSNKYYADSVKGRFTISRDNSKNTLYLQMNSLRAEDTAVYYCARDGTIFGVLLG | |
| | | DYWGQGTLVTVSS | |
| iPS:335941 | 2F11 | QVQLVESGGGVVQPGRSLRLSCAASGFTFSSYGMHWVRQAPGKGLEWVAVIWY | 161 |
| | | DGSNKYYADSVKGRFTISSDNSKNTLYLQMNSLRAEDTAVYYCARDRTIFGVVLD | |
| | | YWGQGTLVTVSS | |
| iPS:335970 | 5C2 | QVPLVQSGAEVKKPGASVKVSCKASGYTFTGYYIHWVRQAPGQGLEWMGWINP | 162 |
| | | DSGGTDYSQRFQGRFTMTRDTSISTAYMELNRLRSDDTAVYYCAREATIFGMVIV | |
| | | PFDYWGQGTLVTVSS | |
| iPS:335978 | 13H12 | QVPLVQSGAEVKKPGASVKVSCKASGYTFTGYYIHWVRQAPGQGLEWMGWINP | 163 |
| | | DSGGTDYSQRFQGRFTMTRDTSISTAYMELSRLRSDDTAVYYCAREATIFGMVIVP | |
| | | FDYWGQGTLVTVSS | |
| iPS:335986 | 11C1 | QVPLVQSGAEVKKPGASVKVSCKASGYTFTGYYIHWVRQAPGQGLVWMGWISP | 164 |
| | | NSGDTSYAQKFQDRVTMTRDTSISTAYMELSRLRSDDTAVYFCTREATIFGMLIVP | |
| | | FDYWGQGTLVTVSS | |
| iPS:335994 | 12H11 | QVPLVQSGAEVKKPGASVKVSCKASGYTFTGYYIHWVRQAPGQGLEWMGWISPN | 165 |
| | | NGDTNYAQKFQDRVTMTRDTSISTAYMELSRLRSDDTAVYYCTREATIFGMLIVP | |
| | | FDYWGQGTLVTVSS | |
| iPS:336024 | 18E3 | QVQLQESGPGLVKPSQTLSLTCTVSGDSISSGGYYWSWIRQHPGKGLEWIGYIYYS | 166 |
| | | GSTYYNPSLKSRVTISVDTSKNQFSLKLSSVTAADTAVYYCARDRITIFGVVMGGG | |
| | | MDVWGQGTTVTVSS | |
| iPS:336041 | 2G10_LC1 | QVQLVESGGGVVQPGRSLRLSCAASGFTFSNYGMHWVRQAPGEGLEWVAAIWF | 167 |
| | | DGSDKYYADSVKGRFTISRDNSKNTLYLQMNSLRAEDTAVYYCARDQAIFGVVP | |
| | | DYWGQGTLVTVSS | |
| iPS:336077 | 4H9.004 | QVQLVESGGGVVQPGRSLRLSCAASGFTFSYFGMHWVRQAPGKGLEWVAVIWY | 168 |
| | | DGSNKYYADSVKGRFTISRDNSKNTLYLQMNSLRAEDTAVYYCARDGTIFGVLLG | |
| | | DYWGQGTLVTVSS | |
| iPS:336088 | 6A5.004 | QVQLVESGGGVVQPGRSLRLSCAASGFSFSSYGMHWVRQPPGKGLEWVAAIWYD | 169 |
| | | GNNKYYADSVKGRFTISRDSSKNTLYLQMNSLRAEDTAVYYCARDRTIFGVVLEY | |
| | | LGQGTLVTVSS | |
| iPS:336099 | 17H11.004 | QVQLQESGPGLVKPSETLSLTCTVSQGSISSYYWSWIRQPAGKGLEWIGRIYTSGST | 170 |
| | | NYNPSLKSRVTMSIDTSKNQFSLKLNSVTAADTAVYYCARDVAVAGFDYWGQGT | |
| | | LVTVSS | |
| iPS:359621 | 17H11.004.001 | QVQLQESGPGLVKPSETLSLTCTVSGGSISSYYWSWIRQPAGKGLEWIGRIYTSGST | 171 |
| | | NYNPSLKSRVTMSIDTSKNQFSLKLNSVTAADTAVYYCARDVAVAGFDYWGQGT | |
| | | LVTVSS | |
| iPS:359781 | 4H9.004.001 | QVQLVESGGGVVQPGRSLRLSCAASGFTFSYFGMHWVRQAPGKGLEWVAVIWY | 172 |
| | | DASNKYYADAVKGRFTISRDNSKNTLYLQMNSLRAEDTAVYYCARDGTIFGVLL | |
| | | GDYWGQGTLVTVSS | |
| iPS:360929 | 4H9.004.002 | QVQLVESGGGVVQPGRSLRLSCAASGFTFSYFGMHWVRQAPGKGLEWVAVIWY | 173 |
| | | DASNKYYADSVKGRFTISRDNSKNTLYLQMNSLRAEDTAVYYCARDGTIFGVLLG | |
| | | DYWGQGTLVTVSS | |
| iPS:360936 | 4H9.004.003 | QVQLVESGGGVVQPGRSLRLSCAASGFTFSYFGMHWVRQAPGKGLEWVAVIWY | 174 |
| | | DGSNKYYADAVKGRFTISRDNSKNTLYLQMNSLRAEDTAVYYCARDGTIFGVLL | |
| | | GDYWGQGTLVTVSS | |
| iPS:360943 | 4H9.004.004 | QVQLVESGGGVVQPGRSLRLSCAASGFTFSYFGMHWVRQAPGKGLEWVAVIWY | 175 |
| | | DASNKYYADAVKGRFTISRDNSKNTLYLQMNSLRAEDTAVYYCARDGTIFGVLL | |
| | | GDYWGQGTLVTVSS | |
| iPS:360949 | 4H9.004.005 | QVQLVESGGGVVQPGRSLRLSCAASGFTFSYFGMHWVRQAPGKGLEWVAVIWY | 176 |
| | | DASNKYYADAVKGRFTISRDNSKNTLYLQMNSLRAEDTAVYYCARDGTIFGVLL | |
| | | GDYWGQGTLVTVSS | |
| iPS:359761 | 4H9.004.006 | QVQLVESGGGVVQPGRSLRLSCAASGFTFSYFGMHWVRQAPGKGLEWVAVIWY | 177 |
| | | DASNKYYADAVKGRFTISRDNSKNTLYLQMNSLRAEDTAVYYCARDRTIFGVLL | |
| | | GDYWGQGTLVTVSS | |
| iPS:360955 | 4B1.010 | QVQLVESGGGVVQPGRSLRLSCAASGFTFSNFGMHWVRQAPGKGLEWVAVIWY | 178 |
| | | DASNENYADAVKGRFTISRDNSKNTLYLQMNSLRVADTAVYYCARDGTIFGVVL | |
| | | GDYWGQGTLVTVSS | |
| iPS:360962 | 4B1.011 | QVQLVESGGGVVQPGRSLRLSCAASGFTFSNFGMHWVRQAPGKGLEWVAVIWY | 179 |
| | | DASNENYADAVKGRFTISRDNSKNTLYLQMNSLRVADTAVYYCARDGTIFGVVL | |
| | | GDYWGQGTLVTVSS | |
| iPS:360968 | 4B1.012 | QVQLVESGGGVVQPGRSLRLSCAASGFTFSNFGMHWVRQAPGKGLEWVAVIWY | 180 |
| | | DASNENYADAVKGRFTISRDNSKNTLYLQMNSLRVADTAVYYCARDGTIFGVVL | |
| | | GDYWGQGTLVTVSS | |
| iPS:360974 | 4B1.013 | QVQLVESGGGVVQPGRSLRLSCAASGFTFSNFGMHWVRQAPGKGLEWVAVIWY | 181 |
| | | DASNENYADSVKGRFTISRDNSKNTLYLHMNSLRVADTAVYYCARDGTIFGVVL | |
| | | GDYWGQGTLVTVSS | |
| iPS:360978 | 4B1.014 | QVQLVESGGGVVQPGRSLRLSCAASGFTFSNFGMHWVRQAPGKGLEWVAVIWY | 182 |
| | | DGSNENYADAVKGRFTISRDNSKNTLYLHMNSLRVADTAVYYCARDGTIFGVVL | |
| | | GDYWGQGTLVTVSS | |
| iPS:359785 | 4B1.015 | QVQLVESGGGVVQPGRSLRLSCAASGFTFSNFGMHWVRQAPGKGLEWVAVIWY | 183 |
| | | DASNENYADAVKGRFTISRDNSKNTLYLQMNSLRVEDTAVYYCARDRTIFGVVL | |
| | | GDYWGQGTLVTVSS | |
| iPS:360982 | 4B1.016 | QVQLVESGGGVVQPGRSLRLSCAASGFTFSNFGMHWVRQAPGKGLEWVAVIWY | 184 |
| | | DASNENYADAVKGRFTISRDNSKNTLYLQMNSLRVEDTAVYYCARDGTIFGVVL | |
| | | GDYWGQGTLVTVSS | |
| iPS:335922 | 18F2 | QVQLVESGGGVVQPGRSLRLSCAASGFTFSNFGMHWVRQAPGKGLEWVAVIWY | 185 |
| | | DGSNENYADSVKGRFTISRDNSKNTLYLHMNSLRAADTAVYYCARDGTIFGVVL | |
| | | GDYWGQGTLVTVSS | |
| iPS:360986 | 18F2.002 | QVQLVESGGGVVQPGRSLRLSCAASGFTFSNFGMHWVRQAPGKGLEWVAVIWY | 186 |
| | | DASNENYADAVKGRFTISRDNSKNTLYLQMNSLRAADTAVYYCARDGTIFGVVL | |
| | | GDYWGQGTLVTVSS | |
| iPS:360995 | 18F2.003 | QVQLVESGGGVVQPGRSLRLSCAASGFTFSNFGMHWVRQAPGKGLEWVAVIWY | 187 |
| | | DASNENYADAVKGRFTISRDNSKNTLYLHMNSLRAADTAVYYCARDGTIFGVVL | |
| | | GDYWGQGTLVTVSS | |
| iPS:360999 | 18F2.004 | QVQLVESGGGVVQPGRSLRLSCAASGFTFSNFGMHWVRQAPGKGLEWVAVIWY | 188 |
| | | DASNENYADAVKGRFTISRDNSKNTLYLQMNSLRAADTAVYYCARDGTIFGVVL | |
| | | GDYWGQGTLVTVSS | |
| iPS:361005 | 18F2.005 | QVQLVESGGGVVQPGRSLRLSCAASGFTFSNFGMHWVRQAPGKGLEWVAVIWY | 189 |
| | | DASNENYADAVKGRFTISRDNSKNTLYLQMNSLRAADTAVYYCARDGTIFGVVL | |
| | | GDYWGQGTLVTVSS | |
| iPS:361009 | 18F2.006 | QVQLVESGGGVVQPGRSLRLSCAASGFTFSNFGMHWVRQAPGKGLEWVAVIWY | 190 |
| | | DASNENYADAVKGRFTISRDNSKNTLYLHMNSLRAADTAVYYCARDGTIFGVVL | |
| | | GDYWGQGTLVTVSS | |
| iPS:361013 | 18F2.007 | QVQLVESGGGVVQPGRSLRLSCAASGFTFSNFGMHWVRQAPGKGLEWVAVIWY | 191 |
| | | DASNENYADAVKGRFTISRDNSKNTLYLHMNSLRAADTAVYYCARDGTIFGVVL | |
| | | GDYWGQGTLVTVSS | |
| iPS:361017 | 18F2.008 | QVQLVESGGGVVQPGRSLRLSCAASGFTFSNFGMHWVRQAPGKGLEWVAVIWY | 192 |
| | | DASNENYADAVKGRFTISRDNSKNTLYLQMNSLRAADTAVYYCARDGTIFGVVL | |
| | | GDYWGQGTLVTVSS | |
| iPS:361021 | 18F2.009 | QVQLVESGGGVVQPGRSLRLSCAASGFTFSNFGMHWVRQAPGKGLEWVAVIWY | 193 |
| | | DGSNENYADAVKGRFTISRDNSKNTLYLQMNSLRAADTAVYYCARDGTIFGVVL | |
| | | GDYWGQGTLVTVSS | |
| iPS:361028 | 18F2.010 | QVQLVESGGGVVQPGRSLRLSCAASGFTFSNFGMHWVRQAPGKGLEWVAVIWY | 194 |
| | | DASNENYADSVKGRFTISRDNSKNTLYLQMNSLRAADTAVYYCARDGTIFGVVL | |
| | | GDYWGQGTLVTVSS | |
| iPS:360535 | 18F2.011 | QVQLVESGGGVVQPGRSLRLSCAASGFTFSNFGMHWVRQAPGKGLEWVAVIWY | 195 |
| | | DASNENYADAVKGRFTISRDNSKNTLYLQMNSLRAEDTAVYYCARDRTIFGVVL | |
| | | GDYWGQGTLVTVSS | |
| iPS:361035 | 18F2.012 | QVQLVESGGGVVQPGRSLRLSCAASGFTFSNFGMHWVRQAPGKGLEWVAVIWY | 196 |
| | | DASNENYADAVKGRFTISRDNSKNTLYLQMNSLRAEDTAVYYCARDGTIFGVVL | |
| | | GDYWGQGTLVTVSS | |
| iPS:359940 | 2F11.002 | QVQLVESGGGVVQPGRSLRLSCAASGFTFSSYGMHWVRQAPGKGLEWVAVIWY | 197 |
| | | DASNKYYADAVKGRFTISRDNSKNTLYLQMNSLRAEDTAVYYCARDRTIFGVVL | |
| | | DYWGQGTLVTVSS | |
| iPS:361039 | 2F11.003 | QVQLVESGGGVVQPGRSLRLSCAASGFTFSSYGMHWVRQAPGKGLEWVAVIWY | 198 |
| | | DASNKYYADAVKGRFTISSDNSKNTLYLQMNSLRAEDTAVYYCARDRTIFGVVL | |
| | | DYWGQGTLVTVSS | |
| iPS:361043 | 2F11.004 | QVQLVESGGGVVQPGRSLRLSCAASGFTFSSYGMHWVRQAPGKGLEWVAVIWY | 199 |
| | | DASNKYYADAVKGRFTISRDNSKNTLYLQMNSLRAEDTAVYYCARDRTIFGVVL | |
| | | DYWGQGTLVTVSS | |
| iPS:361049 | 2F11.005 | QVQLVESGGGVVQPGRSLRLSCAASGFTFSSYGMHWVRQAPGKGLEWVAVIWY | 200 |
| | | DASNKYYADAVKGRFTISRDNSKNTLYLQMNSLRAEDTAVYYCARDRTIFGVVL | |
| | | DYWGQGTLVTVSS | |
| iPS:361055 | 2F11.006 | QVQLVESGGGVVQPGRSLRLSCAASGFTFSSYGMHWVRQAPGKGLEWVAVIWY | 201 |
| | | DASNKYYADAVKGRFTISSDNSKNTLYLQMNSLRAEDTAVYYCARDRTIFGVVL | |
| | | DYWGQGTLVTVSS | |
| iPS:361059 | 2F11.007 | QVQLVESGGGVVQPGRSLRLSCAASGFTFSSYGMHWVRQAPGKGLEWVAVIWY | 202 |
| | | DASNKYYADAVKGRFTISSDNSKNTLYLQMNSLRAEDTAVYYCARDRTIFGVVL | |
| | | DYWGQGTLVTVSS | |
| iPS:361063 | 2F11.008 | QVQLVESGGGVVQPGRSLRLSCAASGFTFSSYGMHWVRQAPGKGLEWVAVIWY | 203 |
| | | DASNKYYADAVKGRFTISRDNSKNTLYLQMNSLRAEDTAVYYCARDRTIFGVVL | |
| | | DYWGQGTLVTVSS | |
| iPS:359949 | 2F11.009 | QVQLVESGGGVVQPGRSLRLSCAASGFTFSSYGMHWVRQAPGKGLEWVAVIWY | 204 |
| | | DASNKYYADSVKGRFTISRDNSKNTLYLQMNSLRAEDTAVYYCARDRTIFGVVLD | |
| | | YWGQGTLVTVSS | |
| iPS:359956 | 2F11.010 | QVQLVESGGGVVQPGRSLRLSCAASGFTFSSYGMHWVRQAPGKGLEWVAVIWY | 205 |
| | | DGSNKYYADAVKGRFTISRDNSKNTLYLQMNSLRAEDTAVYYCARDRTIFGVVL | |
| | | DYWGQGTLVTVSS | |
| iPS:359865 | 6H1.002 | QVQLVESGGGVVQPGRSLRLSCAASGFTFSYFGMHWVRQAPGKGLEWVAVIWY | 206 |
| | | DASNKYYADAVKGRFTISRDNSKNTLYLQMNSLRAEDTAVYYCARDGTIFGVLL | |
| | | GDYWGQGTLVTVSS | |
| iPS:359869 | 6H1.003 | QVQLVESGGGVVQPGRSLRLSCAASGFTFSYFGMHWVRQAPGKGLEWVAVIWY | 207 |
| | | DASNKYYADAVKGRFTISRDNSKNTLYLQMNSLRAEDTAVYYCARDRTIFGVLL | |
| | | GDYWGQGTLVTVSS | |
| iPS:359873 | 6H1.004 | QVQLVESGGGVVQPGRSLRLSCAASGFTFSYFGMHWVRQAPGKGLEWVAVIWY | 208 |
| | | DASNKYYADAVKGRFTISRDNSKNTLYLQMNSLRAEDTAVYYCARDGTIFGVLL | |
| | | GDYWGQGTLVTVSS | |
| iPS:359877 | 6H1.005 | QVQLVESGGGVVQPGRSLRLSCAASGFTFSYFGMHWVRQAPGKGLEWVAVIWY | 209 |
| | | DASNKYYADAVKGRFTISRDNSKNTLYLQMNSLRAEDTAVYYCARDRTIFGVLL | |
| | | GDYWGQGTLVTVSS | |
| iPS:361067 | 6H1.006 | QVQLVESGGGVVQPGRSLRLSCAASGFTFSYFGMHWVRQAPGKGLEWVAVIWY | 210 |
| | | DGSNKYYADSVKGRFTISRDNSKNTLYLQMNSLRAEDTAVYYCARDGTIFGVLLG | |
| | | DYWGQGTLVTVSS | |
| iPS:361071 | 6H1.007 | QVQLVESGGGVVQPGRSLRLSCAASGFTFSYFGMHWVRQAPGKGLEWVAVIWY | 211 |
| | | DASNKYYADSVKGRFTISRDNSKNTLYLQMNSLRAEDTAVYYCARDGTIFGVLLG | |
| | | DYWGQGTLVTVSS | |
| iPS:361075 | 6H1.008 | QVQLVESGGGVVQPGRSLRLSCAASGFTFSYFGMHWVRQAPGKGLEWVAVIWY | 212 |
| | | DGSNKYYADAVKGRFTISRDNSKNTLYLQMNSLRAEDTAVYYCARDGTIFGVLL | |
| | | GDYWGQGTLVTVSS | |
| iPS:361079 | 6A5.004.001 | QVQLVESGGGVVQPGRSLRLSCAASGFSFSSYGMHWVRQPPGKGLEWVAAIWYD | 213 |
| | | ANNKYYADAVKGRFTISRDSSKNTLYLQMNSLRAEDTAVYYCARDRTIFGVVLE | |
| | | YWGQGTLVTVSS | |
| iPS:361085 | 6A5.004.002 | QVQLVESGGGVVQPGRSLRLSCAASGFSFSSYGMHWVRQPPGKGLEWVAAIWYD | 214 |
| | | ANNKYYADAVKGRFTISRDSSKNTLYLQMNSLRAEDTAVYYCARDRTIFGVVLE | |
| | | YWGQGTLVTVSS | |
| iPS:361091 | 6A5.004.003 | QVQLVESGGGVVQPGRSLRLSCAASGFSFSSYGMHWVRQPPGKGLEWVAAIWYD | 215 |
| | | ANNKYYADAVKGRFTISRDSSKNTLYLQMNSLRAEDTAVYYCARDRTIFGVVLE | |
| | | YLGQGTLVTVSS | |
| iPS:361095 | 6A5.004.004 | QVQLVESGGGVVQPGRSLRLSCAASGFSFSSYGMHWVRQPPGKGLEWVAAIWYD | 216 |
| | | ANNKYYADAVKGRFTISRDSSKNTLYLQMNSLRAEDTAVYYCARDRTIFGVVLE | |
| | | YWGQGTLVTVSS | |
| iPS:361101 | 6A5.004.005 | QVQLVESGGGVVQPGRSLRLSCAASGFSFSSYGMHWVRQPPGKGLEWVAAIWYD | 217 |
| | | ANNKYYADAVKGRFTISRDSSKNTLYLQMNSLRAEDTAVYYCARDRTIFGVVLE | |
| | | YLGQGTLVTVSS | |
| iPS:361105 | 6A5.004.006 | QVQLVESGGGVVQPGRSLRLSCAASGFSFSSYGMHWVRQPPGKGLEWVAAIWYD | 218 |
| | | ANNKYYADAVKGRFTISRDSSKNTLYLQMNSLRAEDTAVYYCARDRTIFGVVLE | |
| | | YLGQGTLVTVSS | |
| iPS:361109 | 6A5.004.007 | QVQLVESGGGVVQPGRSLRLSCAASGFSFSSYGMHWVRQPPGKGLEWVAAIWYD | 219 |
| | | ANNKYYADAVKGRFTISRDSSKNTLYLQMNSLRAEDTAVYYCARDRTIFGVVLE | |
| | | YWGQGTLVTVSS | |
| iPS:361113 | 6A5.004.008 | QVQLVESGGGVVQPGRSLRLSCAASGFSFSSYGMHWVRQPPGKGLEWVAAIWYD | 220 |
| | | GNNKYYADAVKGRFTISRDSSKNTLYLQMNSLRAEDTAVYYCARDRTIFGVVLE | |
| | | YWGQGTLVTVSS | |
| iPS:361120 | 6A5.004.009 | QVQLVESGGGVVQPGRSLRLSCAASGFSFSSYGMHWVRQPPGKGLEWVAAIWYD | 221 |
| | | ANNKYYADSVKGRFTISRDSSKNTLYLQMNSLRAEDTAVYYCARDRTIFGVVLEY | |
| | | WGQGTLVTVSS | |
| iPS:359896 | 6A5.004.010 | QVQLVESGGGVVQPGRSLRLSCAASGFSFSSYGMHWVRQPPGKGLEWVAAIWYD | 222 |
| | | ANNKYYADAVKGRFTISRDSSKNTLYLQMNSLRAEDTAVYYCARDRTIFGVVLE | |
| | | YWGQGTLVTVSS | |
| iPS:359890 | 6A5.004.011 | QVQLVESGGGVVQPGRSLRLSCAASGFSFSSYGMHWVRQPPGKGLEWVAAIWYD | 223 |
| | | ANNKYYADAVKGRFTISRDSSKNTLYLQMNSLRAEDTAVYYCARDRTIFGVVLE | |
| | | YLGQGTLVTVSS | |
| iPS:359567 | 2A11.002 | QVQLVESGGGVVQPGRSLRLSCAASGFSFSSYGMHWVRQAPGKGLEWVAVIWY | 224 |
| | | DASNKYYEDAVKGRFTISRDNSKNTLFLQVNSLRAEDTAVYYCARGITIFGHGFEY | |
| | | WGQGTLVTVSS | |
| iPS:335965 | 2A11.003 | QVQLVESGGGVVQPGRSLRLSCAASGFSFSSYGMHWVRQAPGKGLEWVAVIWY | 225 |
| | | DGSNKYYEDSVKGRFTISRDNSKNTLFLQVNSLRAEDTAVYYCARGITIFGHGFEY | |
| | | WGQGTLVTVSS | |
| iPS:361158 | 2A11.004 | QVQLVESGGGVVQPGRSLRLSCAASGFSFSSYGMHWVRQAPGKGLEWVAVIWY | 226 |
| | | DGSNKYYEDAVKGRFTISRDNSKNTLFLQVNSLRAEDTAVYYCARGITIFGHGFEY | |
| | | WGQGTLVTVSS | |
| iPS:361165 | 2A11.005 | QVQLVESGGGVVQPGRSLRLSCAASGFSFSSYGMHWVRQAPGKGLEWVAVIWY | 227 |
| | | DASNKYYEDSVKGRFTISRDNSKNTLFLQVNSLRAEDTAVYYCARGITIFGHGFEY | |
| | | WGQGTLVTVSS | |
| iPS:361172 | 2G10_LC1.003 | QVQLVESGGGVVQPGRSLRLSCAASGFTFSNYGMHWVRQAPGEGLEWVAAIWF | 228 |
| | | DASDKYYADAVKGRFTISRDNSKNTLYLQMNSLRAEDTAVYYCARDQAIFGVVP | |
| | | DYWGQGTLVTVSS | |
| iPS:361178 | 2G10_LC1.004 | QVQLVESGGGVVQPGRSLRLSCAASGFTFSNYGMHWVRQAPGEGLEWVAAIWF | 229 |
| | | DASDKYYADSVKGRFTISRDNSKNTLYLQMNSLRAEDTAVYYCARDQAIFGVVP | |
| | | DYWGQGTLVTVSS | |
| iPS:361185 | 2G10_LC1.005 | QVQLVESGGGVVQPGRSLRLSCAASGFTFSNYGMHWVRQAPGEGLEWVAAIWF | 230 |
| | | DGSDKYYADAVKGRFTISRDNSKNTLYLQMNSLRAEDTAVYYCARDQAIFGVVP | |
| | | DYWGQGTLVTVSS | |
| iPS:361192 | 2G10_LC1.006 | QVQLVESGGGVVQPGRSLRLSCAASGFTFSNYGMHWVRQAPGEGLEWVAAIWF | 231 |
| | | DASDKYYADAVKGRFTISRDNSKNTLYLQMNSLRAEDTAVYYCARDQAIFGVVP | |
| | | DYWGQGTLVTVSS | |
| iPS:359609 | 2G10_LC1.007 | QVQLVESGGGVVQPGRSLRLSCAASGFTFSNYGMHWVRQAPGKGLEWVAAIWF | 232 |
| | | DASDKYYADAVKGRFTISRDNSKNTLYLQMNSLRAEDTAVYYCARDQAIFGVVP | |
| | | DYWGQGTLVTVSS | |
| iPS:359615 | 2G10_LC1.008 | QVQLVESGGGVVQPGRSLRLSCAASGFTFSNYGMHWVRQAPGKGLEWVAAIWF | 233 |
| | | DASDKYYADAVKGRFTISRDNSKNTLYLQMNSLRAEDTAVYYCARDQAIFGVVP | |
| | | DYWGQGTLVTVSS | |
| iPS:361196 | 2G10_LC1.009 | QVQLVESGGGVVQPGRSLRLSCAASGFTFSNYGMHWVRQAPGEGLEWVAAIWF | 234 |
| | | DASDKYYADAVKGRFTISRDNSKNTLYLQMNSLRAEDTAVYYCARDQAIFGVVP | |
| | | DYWGQGTLVTVSS | |
| iPS:361202 | 2G10_LC1.010 | QVQLVESGGGVVQPGRSLRLSCAASGFTFSNYGMHWVRQAPGKGLEWVAAIWF | 235 |
| | | DASDKYYADAVKGRFTISRDNSKNTLYLQMNSLRAEDTAVYYCARDQAIFGVVP | |
| | | DYWGQGTLVTVSS | |
| iPS:359644 | 18E3.002 | QVQLQESGPGLVKPSETLSLTCTVSGDSISSGGYYWSWIRQPPGKGLEWIGYIYYS | 236 |
| | | GSTYYNPSLKSRVTISVDTSKNQFSLKLSSVTAADTAVYYCARDRITIFGVVMGGG | |
| | | MDVWGQGTTVTVSS | |
| iPS:361206 | 18E3.003 | QVQLQESGPGLVKPSETLSLTCTVSGDSISSGGYYWSWIRQPPGKGLEWIGYIYYS | 237 |
| | | GSTYYNPSLKSRVTISVDTSKNQFSLKLSSVTAADTAVYYCARDRITIFGVVMGGG | |
| | | MDVWGQGTTVTVSS | |
| iPS:359628 | 18E3.004 | QVQLQESGPGLVKPSQTLSLTCTVSGDSISSGGYYWSWIRQPPGKGLEWIGYIYYS | 238 |
| | | GSTYYNPSLKSRVTISVDTSKNQFSLKLSSVTAADTAVYYCARDRITIFGVVMGGG | |
| | | MDVWGQGTTVTVSS | |
| iPS:359637 | 18E3.005 | QVQLQESGPGLVKPSETLSLTCTVSGDSISSGGYYWSWIRQHPGKGLEWIGYIYYS | 239 |
| | | GSTYYNPSLKSRVTISVDTSKNQFSLKLSSVTAADTAVYYCARDRITIFGVVMGGG | |
| | | MDVWGQGTTVTVSS | |
| iPS:361210 | 18E3.006 | QVQLQESGPGLVKPSQTLSLTCTVSGDSISSGGYYWSWIRQHPGKGLEWIGYIYYS | 240 |
| | | GSTYYNPSLKSRVTISVDTSKNQFSLKLSSVTAADTAVYYCARDRITIFGVVMGGG | |
| | | MDVWGQGTTVTVSS | |
| iPS:361214 | 18E3.007 | QVQLQESGPGLVKPSETLSLTCTVSGDSISSGGYYWSWIRQHPGKGLEWIGYIYYS | 241 |
| | | GSTYYNPSLKSRVTISVDTSKNQFSLKLSSVTAADTAVYYCARDRITIFGVVMGGG | |
| | | MDVWGQGTTVTVSS | |
| iPS:361218 | 18E3.008 | QVQLQESGPGLVKPSQTLSLTCTVSGDSISSGGYYWSWIRQPPGKGLEWIGYIYYS | 242 |
| | | GSTYYNPSLKSRVTISVDTSKNQFSLKLSSVTAADTAVYYCARDRITIFGVVMGGG | |
| | | MDVWGQGTTVTVSS | |
| iPS:361222 | 5C2.006 | QVQLVQSGAEVKKPGASVKVSCKASGYTFTGYYIHWVRQAPGQGLEWMGWINP | 243 |
| | | ESGGTDYSQRFQGRVTMTRDTSISTAYMELNRLRSDDTAVYYCAREATIFGMVIV | |
| | | PFDYWGQGTLVTVSS | |
| iPS:361229 | 5C2.007 | QVPLVQSGAEVKKPGASVKVSCKASGYTFTGYYIHWVRQAPGQGLEWMGWINP | 244 |
| | | ESGGTDYSQRFQGRFTMTRDTSISTAYMELNRLRSDDTAVYYCAREATIFGMVIVP | |
| | | FDYWGQGTLVTVSS | |
| iPS:361236 | 5C2.008 | QVQLVQSGAEVKKPGASVKVSCKASGYTFTGYYIHWVRQAPGQGLEWMGWINP | 245 |
| | | DAGGTDYSQRFQGRVTMTRDTSISTAYMELNRLRSDDTAVYYCAREATIFGMVIV | |
| | | PFDYWGQGTLVTVSS | |
| iPS:359685 | 5C2.009 | QVPLVQSGAEVKKPGASVKVSCKASGYTFTGYYIHWVRQAPGQGLEWMGWINP | 246 |
| | | DAGGTDYSQRFQGRFTMTRDTSISTAYMELNRLRSDDTAVYYCAREATIFGMVIV | |
| | | PFDYWGQGTLVTVSS | |
| iPS:361240 | 5C2.010 | QVQLVQSGAEVKKPGASVKVSCKASGYTFTGYYIHWVRQAPGQGLEWMGWINP | 247 |
| | | DSGGTDYSQRFQGRVTMTRDTSISTAYMELNRLRSDDTAVYYCAREATIFGMVIV | |
| | | PFDYWGQGTLVTVSS | |
| iPS:361247 | 5C2.011 | QVQLVQSGAEVKKPGASVKVSCKASGYTFTGYYIHWVRQAPGQGLEWMGWINP | 248 |
| | | DSGGTDYSQRFQGRFTMTRDTSISTAYMELNRLRSDDTAVYYCAREATIFGMVIV | |
| | | PFDYWGQGTLVTVSS | |
| iPS:361254 | 5C2.012 | QVPLVQSGAEVKKPGASVKVSCKASGYTFTGYYIHWVRQAPGQGLEWMGWINP | 249 |
| | | DSGGTDYSQRFQGRVTMTRDTSISTAYMELNRLRSDDTAVYYCAREATIFGMVIV | |
| | | PFDYWGQGTLVTVSS | |
| iPS:361261 | 5C2.013 | QVQLVQSGAEVKKPGASVKVSCKASGYTFTGYYIHWVRQAPGQGLEWMGWINP | 250 |
| | | DAGGTDYSQRFQGRFTMTRDTSISTAYMELNRLRSDDTAVYYCAREATIFGMVIV | |
| | | PFDYWGQGTLVTVSS | |
| iPS:361268 | 5C2.014 | QVPLVQSGAEVKKPGASVKVSCKASGYTFTGYYIHWVRQAPGQGLEWMGWINP | 251 |
| | | DAGGTDYSQRFQGRVTMTRDTSISTAYMELNRLRSDDTAVYYCAREATIFGMVIV | |
| | | PFDYWGQGTLVTVSS | |
| iPS:359669 | 5C2.015 | QVQLVQSGAEVKKPGASVKVSCKASGYTFTGYYIHWVRQAPGQGLEWMGWINP | 252 |
| | | DAGGTDYSQRFQGRVTMTRDTSISTAYMELNSLRSDDTAVYYCAREATIFGMVIV | |
| | | PFDYWGQGTLVTVSS | |
| iPS:359678 | 5C2.016 | QVQLVQSGAEVKKPGASVKVSCKASGYTFTGYYIHWVRQAPGQGLEWMGWINP | 253 |
| | | ESGGTDYSQRFQGRVTMTRDTSISTAYMELNSLRSDDTAVYYCAREATIFGMVIVP | |
| | | FDYWGQGTLVTVSS | |
| iPS:361275 | 11C1.002 | QVPLVQSGAEVKKPGASVKVSCKASGYTFTGYYIHWVRQAPGQGLVWMGWISP | 254 |
| | | NSGETSYAQKFQDRVTMTRDTSISTAYMELSRLRSDDTAVYFCAREATIFGMLIVP | |
| | | FDYWGQGTLVTVSS | |
| iPS:361282 | 11C1.003 | QVPLVQSGAEVKKPGASVKVSCKASGYTFTGYYIHWVRQAPGQGLVWMGWISP | 255 |
| | | NSGDTSYAQKFQDRVTMTRDTSISTAYMELSRLRSDDTAVYFCAREATIFGMLIVP | |
| | | FDYWGQGTLVTVSS | |
| iPS:361289 | 11C1.004 | QVPLVQSGAEVKKPGASVKVSCKASGYTFTGYYIHWVRQAPGQGLVWMGWISP | 256 |
| | | NSGETSYAQKFQDRVTMTRDTSISTAYMELSRLRSDDTAVYFCAREATIFGMLIVP | |
| | | FDYWGQGTLVTVSS | |
| iPS:359712 | 11C1.005 | QVPLVQSGAEVKKPGASVKVSCKASGYTFTGYYIHWVRQAPGQGLVWMGWISP | 257 |
| | | NSGETSYAQKFQDRVTMTRDTSISTAYMELSRLRSDDTAVYFCTREATIFGMLIVP | |
| | | FDYWGQGTLVTVSS | |
| iPS:361293 | 11C1.006 | QVPLVQSGAEVKKPGASVKVSCKASGYTFTGYYIHWVRQAPGQGLVWMGWISP | 258 |
| | | NSGDTSYAQKFQDRVTMTRDTSISTAYMELSRLRSDDTAVYFCTREATIFGMLIVP | |
| | | FDYWGQGTLVTVSS | |
| iPS:361297 | 11C1.007 | QVPLVQSGAEVKKPGASVKVSCKASGYTFTGYYIHWVRQAPGQGLVWMGWISP | 259 |
| | | NSGETSYAQKFQDRVTMTRDTSISTAYMELSRLRSDDTAVYFCTREATIFGMLIVP | |
| | | FDYWGQGTLVTVSS | |
| iPS:361301 | 11C1.008 | QVPLVQSGAEVKKPGASVKVSCKASGYTFTGYYIHWVRQAPGQGLVWMGWISP | 260 |
| | | NSGDTSYAQKFQDRVTMTRDTSISTAYMELSRLRSDDTAVYFCAREATIFGMLIVP | |
| | | FDYWGQGTLVTVSS | |
| iPS:359703 | 11C1.009 | QVQLVQSGAEVKKPGASVKVSCKASGYTFTGYYIHWVRQAPGQGLEWMGWISP | 261 |
| | | NSGETSYAQKFQDRVTMTRDTSISTAYMELSSLRSDDTAVYFCAREATIFGMLIVP | |
| | | FDYWGQGTLVTVSS | |
| iPS:361305 | 11C1.010 | QVQLVQSGAEVKKPGASVKVSCKASGYTFTGYYIHWVRQAPGQGLEWMGWISP | 262 |
| | | NSGETSYAQKFQDRVTMTRDTSISTAYMELSRLRSDDTAVYFCAREATIFGMLIVP | |
| | | FDYWGQGTLVTVSS | |
| iPS:361312 | 13H12.002 | QVPLVQSGAEVKKPGASVKVSCKASGYTFTGYYIHWVRQAPGQGLEWMGWINP | 263 |
| | | ESGGTDYSQRFQGRVTMTRDTSISTAYMELSRLRSDDTAVYYCAREATIFGMVIVP | |
| | | FDYWGQGTLVTVSS | |
| iPS:359744 | 13H12.003 | QVPLVQSGAEVKKPGASVKVSCKASGYTFTGYYIHWVRQAPGQGLEWMGWINP | 264 |
| | | ESGGTDYSQRFQGRFTMTRDTSISTAYMELSRLRSDDTAVYYCAREATIFGMVIVP | |
| | | FDYWGQGTLVTVSS | |
| iPS:361319 | 13H12.004 | QVPLVQSGAEVKKPGASVKVSCKASGYTFTGYYIHWVRQAPGQGLEWMGWINP | 265 |
| | | DSGGTDYSQRFQGRVTMTRDTSISTAYMELSRLRSDDTAVYYCAREATIFGMVIV | |
| | | PFDYWGQGTLVTVSS | |
| iPS:359737 | 13H12.005 | QVQLVQSGAEVKKPGASVKVSCKASGYTFTGYYIHWVRQAPGQGLEWMGWINP | 266 |
| | | ESGGTDYSQRFQGRVTMTRDTSISTAYMELSSLRSDDTAVYYCAREATIFGMVIVP | |
| | | FDYWGQGTLVTVSS | |
| iPS:361326 | 13H12.006 | QVPLVQSGAEVKKPGASVKVSCKASGYTFTGYYIHWVRQAPGQGLEWMGWINP | 267 |
| | | ESGGTDYSQRFQGRVTMTRDTSISTAYMELSRLRSDDTAVYYCAREATIFGMVIVP | |
| | | FDYWGQGTLVTVSS | |
| iPS:361330 | 12H11.002 | QVPLVQSGAEVKKPGASVKVSCKASGYTFTGYYIHWVRQAPGQGLEWMGWISPN | 268 |
| | | QGETNYAQKFQDRVTMTRDTSISTAYMELSRLRSDDTAVYYCAREATIFGMLIVP | |
| | | FDYWGQGTLVTVSS | |
| iPS:361337 | 12H11.003 | QVPLVQSGAEVKKPGASVKVSCKASGYTFTGYYIHWVRQAPGQGLEWMGWISPN | 269 |
| | | QGDTNYAQKFQDRVTMTRDTSISTAYMELSRLRSDDTAVYYCAREATIFGMLIVP | |
| | | FDYWGQGTLVTVSS | |
| iPS:361344 | 12H11.004 | QVPLVQSGAEVKKPGASVKVSCKASGYTFTGYYIHWVRQAPGQGLEWMGWISPN | 270 |
| | | QGETNYAQKFQDRVTMTRDTSISTAYMELSRLRSDDTAVYYCTREATIFGMLIVPF | |
| | | DYWGQGTLVTVSS | |
| iPS:361351 | 12H11.005 | QVPLVQSGAEVKKPGASVKVSCKASGYTFTGYYIHWVRQAPGQGLEWMGWISPN | 271 |
| | | NGETNYAQKFQDRVTMTRDTSISTAYMELSRLRSDDTAVYYCAREATIFGMLIVP | |
| | | FDYWGQGTLVTVSS | |
| iPS:361358 | 12H11.006 | QVPLVQSGAEVKKPGASVKVSCKASGYTFTGYYIHWVRQAPGQGLEWMGWISPN | 272 |
| | | NGDTNYAQKFQDRVTMTRDTSISTAYMELSRLRSDDTAVYYCAREATIFGMLIVP | |
| | | FDYWGQGTLVTVSS | |
| iPS:361365 | 12H11.007 | QVPLVQSGAEVKKPGASVKVSCKASGYTFTGYYIHWVRQAPGQGLEWMGWISPN | 273 |
| | | QGDTNYAQKFQDRVTMTRDTSISTAYMELSRLRSDDTAVYYCTREATIFGMLIVP | |
| | | FDYWGQGTLVTVSS | |
| iPS:361372 | 12H11.008 | QVPLVQSGAEVKKPGASVKVSCKASGYTFTGYYIHWVRQAPGQGLEWMGWISPN | 274 |
| | | NGETNYAQKFQDRVTMTRDTSISTAYMELSRLRSDDTAVYYCTREATIFGMLIVPF | |
| | | DYWGQGTLVTVSS | |
| iPS:361379 | 12H11.009 | QVPLVQSGAEVKKPGASVKVSCKASGYTFTGYYIHWVRQAPGQGLEWMGWISPN | 275 |
| | | QGETNYAQKFQDRVTMTRDTSISTAYMELSRLRSDDTAVYYCAREATIFGMLIVP | |
| | | FDYWGQGTLVTVSS | |
| iPS:361383 | 12H11.010 | QVPLVQSGAEVKKPGASVKVSCKASGYTFTGYYIHWVRQAPGQGLEWMGWISPN | 276 |
| | | NGDTNYAQKFQDRVTMTRDTSISTAYMELSRLRSDDTAVYYCAREATIFGMLIVP | |
| | | FDYWGQGTLVTVSS | |
| iPS:361387 | 12H11.011 | QVPLVQSGAEVKKPGASVKVSCKASGYTFTGYYIHWVRQAPGQGLEWMGWISPN | 277 |
| | | QGDTNYAQKFQDRVTMTRDTSISTAYMELSRLRSDDTAVYYCAREATIFGMLIVP | |
| | | FDYWGQGTLVTVSS | |
| iPS:361391 | 12H11.012 | QVPLVQSGAEVKKPGASVKVSCKASGYTFTGYYIHWVRQAPGQGLEWMGWISPN | 278 |
| | | NGETNYAQKFQDRVTMTRDTSISTAYMELSRLRSDDTAVYYCAREATIFGMLIVP | |
| | | FDYWGQGTLVTVSS | |
| iPS:361395 | 12H11.013 | QVQLVQSGAEVKKPGASVKVSCKASGYTFTGYYIHWVRQAPGQGLEWMGWISP | 279 |
| | | NQGETNYAQKFQDRVTMTRDTSISTAYMELSSLRSDDTAVYYCAREATIFGMLIV | |
| | | PFDYWGQGTLVTVSS | |
| iPS:361402 | 12H11.014 | QVQLVQSGAEVKKPGASVKVSCKASGYTFTGYYIHWVRQAPGQGLEWMGWISP | 280 |
| | | NQGETNYAQKFQDRVTMTRDTSISTAYMELSSLRSDDTAVYYCAREATIFGMLIV | |
| | | PFDYWGQGTLVTVSS | |
| iPS:361406 | 2C2.005.001 | QMQVVQSGAEVKKPGASVKVSCKASGYTFTGYYMHWVRQAPGQGLEWMGWIN | 281 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:361412 | 2C2.005.002 | QMQVVQSGAEVKKPGASVKVSCKASGYTFTGYYMHWVRQAPGQGLEWMGWIN | 282 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:361418 | 2C2.005.003 | QMQVVQSGAEVKKPGASVKVSCKASGYTFTGYYMHWVRQAPGQGLEWMGWIN | 283 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:361424 | 2C2.005.004 | QMQVVQSGAEVKKPGASVKVSCKASGYTFTGYYMHWVRQAPGQGLEWMGWIN | 284 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:361430 | 2C2.005.005 | QMQVVQSGAEVKKPGASVKVSCKASGYTFTGYYMHWVRQAPGQGLEWMGWIN | 285 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:361436 | 2C2.005.006 | QMQVVQSGAEVKKPGASVKVSCKASGYTFTGYYMHWVRQAPGQGLEWMGWIN | 286 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:361442 | 2C2.005.007 | QMQVVQSGAEVKKPGASVKVSCKASGYTFTGYYMHWVRQAPGQGLEWMGWIN | 287 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:361448 | 2C2.005.008 | QMQVVQSGAEVKKPGASVKVSCKASGYTFTGYYMHWVRQAPGQGLEWMGWIN | 288 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:361454 | 2C2.005.009 | QMQVVQSGAEVKKPGASVKVSCKASGYTFTGYYMHWVRQAPGQGLEWMGWIN | 289 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:361460 | 2C2.005.010 | QMQVVQSGAEVKKPGASVKVSCKASGYTFTGYYMHWVRQAPGQGLEWMGWIN | 290 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:361466 | 2C2.005.011 | QMQVVQSGAEVKKPGASVKVSCKASGYTFTGYYMHWVRQAPGQGLEWMGWIN | 291 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:361472 | 2C2.005.012 | QMQVVQSGAEVKKPGASVKVSCKASGYTFTGYYMHWVRQAPGQGLEWMGWIN | 292 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:361478 | 2C2.005.013 | QMQVVQSGAEVKKPGASVKVSCKASGYTFTGYYMHWVRQAPGQGLEWMGWIN | 293 |
| | | PNSGGTNYAQKFQGRVTMTRDTSISTAYMELSSLRSDDTAVYYCARGGDYVFGT | |
| | | YRPHYYYGMDVWGQGTTVTVSS | |
| iPS:361485 | 2C2.005.014 | QMQVVQSGAEVKKPGASVKVSCKASGYTFTGYYMHWVRQAPGQGLEWMGWIN | 294 |
| | | PNSGGTNYAQKFQGRVTMTRDTSISTAYMELSRLRSDDTAVYYCARGGDYVFGT | |
| | | YRPHYYYGMDVWGQGTTVTVSS | |
| iPS:361845 | 18F2.013 | QVQLVESGGGVVQPGRSLRLSCAASGFTFSNFGMHWVRQAPGKGLEWVAVIWY | 295 |
| | | DASNENYADAVKGRFTISRDNSKNTLYLQMNSLRAEDTAVYYCARDRTIFGVVL | |
| | | GDYWGQGTLVTVSS | |
| iPS:361851 | 6H1.009 | QVQLVESGGGVVQPGRSLRLSCAASGFTFSYFGMHWVRQAPGKGLEWVAVIWY | 296 |
| | | DASNKYYADAVKGRFTISRDNSKNTLYLQMNSLRAEDTAVYYCARDRTIFGVLL | |
| | | GDYWGQGTLVTVSS | |
| iPS:361855 | 2C2.005.015 | QMQVVQSGAEVKKPGASVKVSCKASGYTFTGYYMHWVRQAPGQGLEWMGWIN | 297 |
| | | PNSGGTNYAQKFQGRVTMTRDTSISTAYMELSRLRSDDTAVYYCARGGDYVWGT | |
| | | YRPHYYYGMDVWGQGTTVTVSS | |
| iPS:336067 | 5G12.006 | QVQLVESGGGVVQPGRSLRLSCTASGFTFSSYGIHWVRQAPGKGLEWVAVIWYD | 298 |
| | | GSNKFHADSVKGRFTISRDNSKNTLYLQMNSLRAEDSAMYFCARGKVAGMPEAF | |
| | | EIWGQGTKVTVSS | |
| iPS:336169 | 17B11.002 | QMQLVQSGAEVKKPGASVKVSCKASGYTFTSYDINWVRQATGQGLEWMGWMN | 299 |
| | | PNSGNTGYAQKFQGRVTMTRDTSISTAYMELSSLRSQDTAVYYCARGGDYVWGS | |
| | | YRPYYYYYGMDVWGQGTTVTVSS | |
| iPS:361127 | 5G12.006.001 | QVQLVESGGGVVQPGRSLRLSCAASGFTFSSYGIHWVRQAPGKGLEWVAVIWYD | 300 |
| | | ASNKFHADAVKGRFTISRDNSKNTLYLQMNSLRAEDSAMYFCARGKVAGMPEAF | |
| | | EIWGQGTKVTVSS | |
| iPS:361136 | 5G12.006.002 | QVQLVESGGGVVQPGRSLRLSCTASGFTFSSYGIHWVRQAPGKGLEWVAVIWYD | 301 |
| | | ASNKFHADAVKGRFTISRDNSKNTLYLQMNSLRAEDSAMYFCARGKVAGMPEAF | |
| | | EIWGQGTKVTVSS | |
| iPS:361140 | 5G12.006.003 | QVQLVESGGGVVQPGRSLRLSCAASGFTFSSYGIHWVRQAPGKGLEWVAVIWYD | 302 |
| | | ASNKFHADAVKGRFTISRDNSKNTLYLQMNSLRAEDSAMYFCARGKVAGMPEAF | |
| | | EIWGQGTKVTVSS | |
| iPS:359919 | 5G12.006.004 | QVQLVESGGGVVQPGRSLRLSCAASGFTFSSYGIHWVRQAPGKGLEWVAVIWYD | 303 |
| | | ASNKFHADAVKGRFTISRDNSKNTLYLQMNSLRAEDSAMYFCARGKVAGMPEAF | |
| | | EIWGQGTLVTVSS | |
| iPS:361144 | 5G12.006.005 | QVQLVESGGGVVQPGRSLRLSCAASGFTFSSYGIHWVRQAPGKGLEWVAVIWYD | 304 |
| | | ASNKFHADSVKGRFTISRDNSKNTLYLQMNSLRAEDSAMYFCARGKVAGMPEAF | |
| | | EIWGQGTKVTVSS | |
| iPS:361151 | 5G12.006.006 | QVQLVESGGGVVQPGRSLRLSCAASGFTFSSYGIHWVRQAPGKGLEWVAVIWYD | 305 |
| | | GSNKFHADAVKGRFTISRDNSKNTLYLQMNSLRAEDSAMYFCARGKVAGMPEAF | |
| | | EIWGQGTKVTVSS | |
| iPS:361491 | 17B11.002.001 | QMQLVQSGAEVKKPGASVKVSCKASGYTFTSYDINWVRQATGQGLEWMGWMN | 306 |
| | | PNSGGTGYAQKFQGRVTMTRDTSISTAYMELSSLRSQDTAVYYCARGGDYVWGS | |
| | | YRPYYYYYGMDVWGQGTTVTVSS | |
| iPS:361499 | 17B11.002.002 | QMQLVQSGAEVKKPGASVKVSCKASGYTFTSYDINWVRQATGQGLEWMGWMN | 307 |
| | | PNSGNTGYAQKFQGRVTMTRDTSISTAYMELSSLRSQDTAVYYCARGGDYVWGS | |
| | | YRPYYYYYGMDVWGQGTTVTVSS | |
| iPS:361503 | 17B11.002.003 | QMQLVQSGAEVKKPGASVKVSCKASGYTFTSYDINWVRQATGQGLEWMGWMN | 308 |
| | | PNSGGTGYAQKFQGRVTMTRDTSISTAYMELSSLRSQDTAVYYCARGGDYVWGS | |
| | | YRPYYYYYGMDVWGQGTTVTVSS | |
| iPS:361507 | 17B11.002.004 | QMQLVQSGAEVKKPGASVKVSCKASGYTFTSYDINWVRQAPGQGLEWMGWMN | 309 |
| | | PNSGGTGYAQKFQGRVTMTRDTSISTAYMELSSLRSDDTAVYYCARGGDYVWGS | |
| | | YRPYYYYYGMDVWGQGTTVTVSS | |
| iPS:361514 | 17B11.002.005 | QMQLVQSGAEVKKPGASVKVSCKASGYTFTSYDINWVRQATGQGLEWMGWMN | 310 |
| | | PNSGGTGYAQKFQGRVTMTRDTSISTAYMELSSLRSQDTAVYYCARGGDYVWGS | |
| | | YRPYYYYYGMDVWGQGTTVTVSS | |
| iPS:360582 | 17B11.002.006 | QMQLVQSGAEVKKPGASVKVSCKASGYTFTSYDINWVRQAPGQGLEWMGWMN | 311 |
| | | PNSGNTGYAQKFQGRVTMTRDTSISTAYMELSSLRSDDTAVYYCARGGDYVWGS | |
| | | YRPYYYYYGMDVWGQGTTVTVSS | |
| iPS:361518 | 17B11.002.007 | QMQLVQSGAEVKKPGASVKVSCKASGYTFTSYDINWVRQATGQGLEWMGWMN | 312 |
| | | PNSGGTGYAQKFQGRVTMTRDTSISTAYMELSSLRSQDTAVYYCARGGDYVFGS | |
| | | YRPYYYYYGMDVWGQGTTVTVSS | |
| iPS:360570 | 17B11.002.008 | QMQLVQSGAEVKKPGASVKVSCKASGYTFTSYDINWVRQAPGQGLEWMGWMN | 313 |
| | | PNSGGTGYAQKFQGRVTMTRDTSISTAYMELSSLRSDDTAVYYCARGGDYVFGS | |
| | | YRPYYYYYGMDVWGQGTTVTVSS | |
| iPS:362051 | 5G12.005.001 | QVQLVESGGGVVQPGRSLRLSCAASGFTFSSYGIHWVRQAPGKGLEWVAVIWYD | 314 |
| | | ASNKFHADAVKGRFTISRDNSKNTLYLQMNSLRAEDSAMYFCARGKVAGMPEAF | |
| | | EIWGQGTLVTVSS | |

TABLE 12
CDRL1, CDRL2, and CDRL3 Amino Acid Sequences of Example Anti-GIPR Antibodies

| Identifier | Construct | CDRL1 | CDRL2 | CDRL3 |
|---|---|---|---|---|
| iPS: 336175 | 2C2.005 | SGSSSNIGSNTVN | TNNQRPS | ATFDDSLNGPV |
| | | SEQ ID NO: 629 | SEQ ID NO: 786 | SEQ ID NO: 943 |
| iPS: 335914 | 4B1 | RASQDIRDYLG | GASSLQS | LQHNNYPFT |
| | | SEQ ID NO: 630 | SEQ ID NO: 787 | SEQ ID NO: 944 |
| iPS: 335938 | 6H1 | RASQDIRDYLG | GASSLQS | LQHNNYPFT |
| | | SEQ ID NO: 631 | SEQ ID NO: 788 | SEQ ID NO: 945 |
| iPS: 335941 | 2F11 | QASQDISNYLN | DASNLET | QQYDILLT |
| | | SEQ ID NO: 632 | SEQ ID NO: 789 | SEQ ID NO: 946 |
| iPS: 335970 | 5C2 | KSSQSVLSSSNNKNYIA | WASTRES | QQYYSTPWT |
| | | SEQ ID NO: 633 | SEQ ID NO: 790 | SEQ ID NO: 947 |
| iPS: 335978 | 13H12 | KSSQSVLSSSNNKNYIA | WASTRES | QQYYSTPWT |
| | | SEQ ID NO: 634 | SEQ ID NO: 791 | SEQ ID NO: 948 |
| iPS: 335986 | 11C1 | KSSQSVLSSSNNKNYLA | WTSTRDS | QQYYRTPWT |
| | | SEQ ID NO: 635 | SEQ ID NO: 792 | SEQ ID NO: 949 |
| iPS: 335994 | 12H11 | KSSQSVLSSSNNKNYLA | WTSTRDS | QQYYRTPWT |
| | | SEQ ID NO: 636 | SEQ ID NO: 793 | SEQ ID NO: 950 |
| iPS: 336024 | 18E3 | RASQSISYSYLA | GASSRAT | QQYGSSPLT |
| | | SEQ ID NO: 637 | SEQ ID NO: 794 | SEQ ID NO: 951 |
| iPS: 336041 | 2G10_LC1 | RASQSVSSNLA | GAATRAT | QQYNNWPLT |
| | | SEQ ID NO: 638 | SEQ ID NO: 795 | SEQ ID NO: 952 |
| iPS: 336077 | 4H9.004 | RASQGIRNELG | GASSLQS | LQHNSYPFT |
| | | SEQ ID NO: 639 | SEQ ID NO: 796 | SEQ ID NO: 953 |
| iPS: 336088 | 6A5.004 | QASQDITNYLN | DASNLEP | QQYDDLFT |
| | | SEQ ID NO: 640 | SEQ ID NO: 797 | SEQ ID NO: 954 |
| iPS: 336099 | 17H11.004 | RASQGLIIWLA | AASSLQS | QQTNSFPPT |
| | | SEQ ID NO: 641 | SEQ ID NO: 798 | SEQ ID NO: 955 |
| iPS: 359621 | 17H11.004.001 | RASQGLIIWLA | AASSLQS | QQTNSFPPT |
| | | SEQ ID NO: 642 | SEQ ID NO: 799 | SEQ ID NO: 956 |
| iPS: 359781 | 4H9.004.001 | RASQGIRNELG | GASSLQS | LQHNSYPFT |
| | | SEQ ID NO: 643 | SEQ ID NO: 800 | SEQ ID NO: 957 |
| iPS: 360929 | 4H9.004.002 | RASQGIRNELG | GASSLQS | LQHNSYPFT |
| | | SEQ ID NO: 644 | SEQ ID NO: 801 | SEQ ID NO: 958 |
| iPS: 360936 | 4H9.004.003 | RASQGIRNELG | GASSLQS | LQHNSYPFT |
| | | SEQ ID NO: 645 | SEQ ID NO: 802 | SEQ ID NO: 959 |
| iPS: 360943 | 4H9.004.004 | RASQGIRNELG | GASSLQS | LQHNSYPFT |
| | | SEQ ID NO: 646 | SEQ ID NO: 803 | SEQ ID NO: 960 |
| iPS: 360949 | 4H9.004.005 | RASQGIRNELG | GASSLQS | LQHNSYPFT |
| | | SEQ ID NO: 647 | SEQ ID NO: 804 | SEQ ID NO: 961 |
| iPS: 359761 | 4H9.004.006 | RASQGIRNELG | GASSLQS | LQHNSYPFT |
| | | SEQ ID NO: 648 | SEQ ID NO: 805 | SEQ ID NO: 962 |
| iPS: 360955 | 4B1.010 | RASQDIRDYLG | GASSLQS | LQHNNYPFT |
| | | SEQ ID NO: 649 | SEQ ID NO: 806 | SEQ ID NO: 963 |
| iPS: 360962 | 4B1.011 | RASQDIRDYLG | GASSLQS | LQHNNYPFT |
| | | SEQ ID NO: 650 | SEQ ID NO: 807 | SEQ ID NO: 964 |
| iPS: 360968 | 4B1.012 | RASQDIRDYLG | GASSLQS | LQHNNYPFT |
| | | SEQ ID NO: 651 | SEQ ID NO: 808 | SEQ ID NO: 965 |
| iPS: 360974 | 4B1.013 | RASQDIRDYLG | GASSLQS | LQHNNYPFT |
| | | SEQ ID NO: 652 | SEQ ID NO: 809 | SEQ ID NO: 966 |
| iPS: 360978 | 4B1.014 | RASQDIRDYLG | GASSLQS | LQHNNYPFT |
| | | SEQ ID NO: 653 | SEQ ID NO: 810 | SEQ ID NO: 967 |
| iPS: 359785 | 4B1.015 | RASQDIRDYLG | GASSLQS | LQHNNYPFT |
| | | SEQ ID NO: 654 | SEQ ID NO: 811 | SEQ ID NO: 968 |
| iPS: 360982 | 4B1.016 | RASQDIRDYLG | GASSLQS | LQHNNYPFT |
| | | SEQ ID NO: 655 | SEQ ID NO: 812 | SEQ ID NO: 969 |
| iPS: 335922 | 18F2 | RASQDLRNYLG | GASSLQS | LQHNNYPFT |
| | | SEQ ID NO: 656 | SEQ ID NO: 813 | SEQ ID NO: 970 |
| iPS: 360986 | 18F2.002 | RASQDLRNYLG | GASSLQS | LQHNNYPFT |
| | | SEQ ID NO: 657 | SEQ ID NO: 814 | SEQ ID NO: 971 |
| iPS: 360995 | 18F2.003 | RASQDLRNYLG | GASSLQS | LQHNNYPFT |
| | | SEQ ID NO: 658 | SEQ ID NO: 815 | SEQ ID NO: 972 |
| iPS: 360999 | 18F2.004 | RASQDLRNYLG | GASSLQS | LQHNNYPFT |
| | | SEQ ID NO: 659 | SEQ ID NO: 816 | SEQ ID NO: 973 |
| iPS: 361005 | 18F2.005 | RASQDLRNYLG | GASSLQS | LQHNNYPFT |
| | | SEQ ID NO: 660 | SEQ ID NO: 817 | SEQ ID NO: 974 |
| iPS: 361009 | 18F2.006 | RASQDLRNYLG | GASSLQS | LQHNNYPFT |
| | | SEQ ID NO: 661 | SEQ ID NO: 818 | SEQ ID NO: 975 |
| iPS: 361013 | 18F2.007 | RASQDLRNYLG | GASSLQS | LQHNNYPFT |
| | | SEQ ID NO: 662 | SEQ ID NO: 819 | SEQ ID NO: 976 |
| iPS: 361017 | 18F2.008 | RASQDLRNYLG | GASSLQS | LQHNNYPFT |
| | | SEQ ID NO: 663 | SEQ ID NO: 820 | SEQ ID NO: 977 |
| iPS: 361021 | 18F2.009 | RASQDLRNYLG | GASSLQS | LQHNNYPFT |
| | | SEQ ID NO: 664 | SEQ ID NO: 821 | SEQ ID NO: 978 |
| iPS: 361028 | 18F2.010 | RASQDLRNYLG | GASSLQS | LQHNNYPFT |
| | | SEQ ID NO: 665 | SEQ ID NO: 822 | SEQ ID NO: 979 |
| iPS: 360535 | 18F2.011 | RASQDLRNYLG | GASSLQS | LQHNNYPFT |
| | | SEQ ID NO: 666 | SEQ ID NO: 823 | SEQ ID NO: 980 |
| iPS: 361035 | 18F2.012 | RASQDLRNYLG | GASSLQS | LQHNNYPFT |
| | | SEQ ID NO: 667 | SEQ ID NO: 824 | SEQ ID NO: 981 |
| iPS: 359940 | 2F11.002 | QASQDISNYLN | DASNLET | QQYDILLT |
| | | SEQ ID NO: 668 | SEQ ID NO: 825 | SEQ ID NO: 982 |
| iPS: 361039 | 2F11.003 | QASQDISNYLN | DASNLET | QQYDILLT |
| | | SEQ ID NO: 669 | SEQ ID NO: 826 | SEQ ID NO: 983 |
| iPS: 361043 | 2F11.004 | QASQDISNYLN | DASNLET | QQYDILLT |
| | | SEQ ID NO: 670 | SEQ ID NO: 827 | SEQ ID NO: 984 |
| iPS: 361049 | 2F11.005 | QASQDISNYLN | DASNLET | QQYDILLT |
| | | SEQ ID NO: 671 | SEQ ID NO: 828 | SEQ ID NO: 985 |
| iPS: 361055 | 2F11.006 | QASQDISNYLN | DASNLET | QQYDILLT |
| | | SEQ ID NO: 672 | SEQ ID NO: 829 | SEQ ID NO: 986 |
| iPS: 361059 | 2F11.007 | QASQDISNYLN | DASNLET | QQYDILLT |
| | | SEQ ID NO: 673 | SEQ ID NO: 830 | SEQ ID NO: 987 |
| iPS: 361063 | 2F11.008 | QASQDISNYLN | DASNLET | QQYDILLT |
| | | SEQ ID NO: 674 | SEQ ID NO: 831 | SEQ ID NO: 988 |
| iPS: 359949 | 2F11.009 | QASQDISNYLN | DASNLET | QQYDILLT |
| | | SEQ ID NO: 675 | SEQ ID NO: 832 | SEQ ID NO: 989 |
| iPS: 359956 | 2F11.010 | QASQDISNYLN | DASNLET | QQYDILLT |
| | | SEQ ID NO: 676 | SEQ ID NO: 833 | SEQ ID NO: 990 |
| iPS: 359865 | 6H1.002 | RASQDIRDYLG | GASSLQS | LQHNNYPFT |
| | | SEQ ID NO: 677 | SEQ ID NO: 834 | SEQ ID NO: 991 |
| iPS: 359869 | 6H1.003 | RASQDIRDYLG | GASSLQS | LQHNNYPFT |
| | | SEQ ID NO: 678 | SEQ ID NO: 835 | SEQ ID NO: 992 |
| iPS: 359873 | 6H1.004 | RASQDIRDYLG | GASSLQS | LQHNNYPFT |
| | | SEQ ID NO: 679 | SEQ ID NO: 836 | SEQ ID NO: 993 |
| iPS: 359877 | 6H1.005 | RASQDIRDYLG | GASSLQS | LQHNNYPFT |
| | | SEQ ID NO: 680 | SEQ ID NO: 837 | SEQ ID NO: 994 |
| iPS: 361067 | 6H1.006 | RASQDIRDYLG | GASSLQS | LQHNNYPFT |
| | | SEQ ID NO: 681 | SEQ ID NO: 838 | SEQ ID NO: 995 |
| iPS: 361071 | 6H1.007 | RASQDIRDYLG | GASSLQS | LQHNNYPFT |
| | | SEQ ID NO: 682 | SEQ ID NO: 839 | SEQ ID NO: 996 |
| iPS: 361075 | 6H1.008 | RASQDIRDYLG | GASSLQS | LQHNNYPFT |
| | | SEQ ID NO: 683 | SEQ ID NO: 840 | SEQ ID NO: 997 |
| iPS: 361079 | 6A5.004.001 | QASQDITNYLN | DASNLEP | QQYDDLFT |
| | | SEQ ID NO: 684 | SEQ ID NO: 841 | SEQ ID NO: 998 |
| iPS: 361085 | 6A5.004.002 | QASQDITNYLN | DASNLEP | QQYDDLFT |
| | | SEQ ID NO: 685 | SEQ ID NO: 842 | SEQ ID NO: 999 |
| iPS: 361091 | 6A5.004.003 | QASQDITNYLN | DASNLEP | QQYDDLFT |
| | | SEQ ID NO: 686 | SEQ ID NO: 843 | SEQ ID NO: 1000 |
| iPS: 361095 | 6A5.004.004 | QASQDITNYLN | DASNLEP | QQYDDLFT |
| | | SEQ ID NO: 687 | SEQ ID NO: 844 | SEQ ID NO: 1001 |
| iPS: 361101 | 6A5.004.005 | QASQDITNYLN | DASNLEP | QQYDDLFT |
| | | SEQ ID NO: 688 | SEQ ID NO: 845 | SEQ ID NO: 1002 |
| iPS: 361105 | 6A5.004.006 | QASQDITNYLN | DASNLEP | QQYDDLFT |
| | | SEQ ID NO: 689 | SEQ ID NO: 846 | SEQ ID NO: 1003 |
| iPS: 361109 | 6A5.004.007 | QASQDITNYLN | DASNLEP | QQYDDLFT |
| | | SEQ ID NO: 690 | SEQ ID NO: 847 | SEQ ID NO: 1004 |
| iPS: 361113 | 6A5.004.008 | QASQDITNYLN | DASNLEP | QQYDDLFT |
| | | SEQ ID NO: 691 | SEQ ID NO: 848 | SEQ ID NO: 1005 |
| iPS: 361120 | 6A5.004.009 | QASQDITNYLN | DASNLEP | QQYDDLFT |
| | | SEQ ID NO: 692 | SEQ ID NO: 849 | SEQ ID NO: 1006 |
| iPS: 359896 | 6A5.004.010 | QASQDITNYLN | DASNLEP | QQYDDLFT |
| | | SEQ ID NO: 693 | SEQ ID NO: 850 | SEQ ID NO: 1007 |
| iPS: 359890 | 6A5.004.011 | QASQDITNYLN | DASNLEP | QQYDDLFT |
| | | SEQ ID NO: 694 | SEQ ID NO: 851 | SEQ ID NO: 1008 |
| iPS: 359567 | 2A11.002 | RASQIFTSTYLA | GASSRAT | QQYGSSPR |
| | | SEQ ID NO: 695 | SEQ ID NO: 852 | SEQ ID NO: 1009 |
| iPS: 335965 | 2A11.003 | RASQIFTSTYLA | GASSRAT | QQYGSSPR |
| | | SEQ ID NO: 696 | SEQ ID NO: 853 | SEQ ID NO: 1010 |
| iPS: 361158 | 2A11.004 | RASQIFTSTYLA | GASSRAT | QQYGSSPR |
| | | SEQ ID NO: 697 | SEQ ID NO: 854 | SEQ ID NO: 1011 |
| iPS: 361165 | 2A11.005 | RASQIFTSTYLA | GASSRAT | QQYGSSPR |
| | | SEQ ID NO: 698 | SEQ ID NO: 855 | SEQ ID NO: 1012 |
| iPS: 361172 | 2G10_LC1.003 | RASQSVSSNLA | GAATRAT | QQYNNWPLT |
| | | SEQ ID NO: 699 | SEQ ID NO: 856 | SEQ ID NO: 1013 |
| iPS: 361178 | 2G10_LC1.004 | RASQSVSSNLA | GAATRAT | QQYNNWPLT |
| | | SEQ ID NO: 700 | SEQ ID NO: 857 | SEQ ID NO: 1014 |
| iPS: 361185 | 2G10_LC1.005 | RASQSVSSNLA | GAATRAT | QQYNNWPLT |
| | | SEQ ID NO: 701 | SEQ ID NO: 858 | SEQ ID NO: 1015 |
| iPS: 361192 | 2G10_LC1.006 | RASQSVSSNLA | GAATRAT | QQYNNWPLT |
| | | SEQ ID NO: 702 | SEQ ID NO: 859 | SEQ ID NO: 1016 |
| iPS: 359609 | 2G10_LC1.007 | RASQSVSSNLA | GAATRAT | QQYNNFPLT |
| | | SEQ ID NO: 703 | SEQ ID NO: 860 | SEQ ID NO: 1017 |
| iPS: 359615 | 2G10_LC1.008 | RASQSVSSNLA | GAATRAT | QQYNNYPLT |
| | | SEQ ID NO: 704 | SEQ ID NO: 861 | SEQ ID NO: 1018 |
| iPS: 361196 | 2G10_LC1.009 | RASQSVSSNLA | GAATRAT | QQYNNWPLT |
| | | SEQ ID NO: 705 | SEQ ID NO: 862 | SEQ ID NO: 1019 |
| iPS: 361202 | 2G10_LC1.010 | RASQSVSSNLA | GAATRAT | QQYNNWPLT |
| | | SEQ ID NO: 706 | SEQ ID NO: 863 | SEQ ID NO: 1020 |
| iPS: 359644 | 18E3.002 | RASQSISYSYLA | GASSRAT | QQYGSSPLT |
| | | SEQ ID NO: 707 | SEQ ID NO: 864 | SEQ ID NO: 1021 |
| iPS: 361206 | 18E3.003 | RASQSISYSYLA | GASSRAT | QQYGSSPLT |
| | | SEQ ID NO: 708 | SEQ ID NO: 865 | SEQ ID NO: 1022 |
| iPS: 359628 | 18E3.004 | RASQSISYSYLA | GASSRAT | QQYGSSPLT |
| | | SEQ ID NO: 709 | SEQ ID NO: 866 | SEQ ID NO: 1023 |
| iPS: 359637 | 18E3.005 | RASQSISYSYLA | GASSRAT | QQYGSSPLT |
| | | SEQ ID NO: 710 | SEQ ID NO: 867 | SEQ ID NO: 1024 |
| iPS: 361210 | 18E3.006 | RASQSISYSYLA | GASSRAT | QQYGSSPLT |
| | | SEQ ID NO: 711 | SEQ ID NO: 868 | SEQ ID NO: 1025 |
| iPS: 361214 | 18E3.007 | RASQSISYSYLA | GASSRAT | QQYGSSPLT |
| | | SEQ ID NO: 712 | SEQ ID NO: 869 | SEQ ID NO: 1026 |
| iPS: 361218 | 18E3.008 | RASQSISYSYLA | GASSRAT | QQYGSSPLT |
| | | SEQ ID NO: 713 | SEQ ID NO: 870 | SEQ ID NO: 1027 |
| iPS: 361222 | 5C2.006 | KSSQSVLSSSNNKNYIA | WASTRES | QQYYSTPWT |
| | | SEQ ID NO: 714 | SEQ ID NO: 871 | SEQ ID NO: 1028 |
| iPS: 361229 | 5C2.007 | KSSQSVLSSSNNKNYIA | WASTRES | QQYYSTPWT |
| | | SEQ ID NO: 715 | SEQ ID NO: 872 | SEQ ID NO: 1029 |
| iPS: 361236 | 5C2.008 | KSSQSVLSSSNNKNYIA | WASTRES | QQYYSTPWT |
| | | SEQ ID NO: 716 | SEQ ID NO: 873 | SEQ ID NO: 1030 |
| iPS: 359685 | 5C2.009 | KSSQSVLSSSNNKNYIA | WASTRES | QQYYSTPWT |
| | | SEQ ID NO: 717 | SEQ ID NO: 874 | SEQ ID NO: 1031 |
| iPS: 361240 | 5C2.010 | KSSQSVLSSSNNKNYIA | WASTRES | QQYYSTPWT |
| | | SEQ ID NO: 718 | SEQ ID NO: 875 | SEQ ID NO: 1032 |
| iPS: 361247 | 5C2.011 | KSSQSVLSSSNNKNYIA | WASTRES | QQYYSTPWT |
| | | SEQ ID NO: 719 | SEQ ID NO: 876 | SEQ ID NO: 1033 |
| iPS: 361254 | 5C2.012 | KSSQSVLSSSNNKNYIA | WASTRES | QQYYSTPWT |
| | | SEQ ID NO: 720 | SEQ ID NO: 877 | SEQ ID NO: 1034 |
| iPS: 361261 | 5C2.013 | KSSQSVLSSSNNKNYIA | WASTRES | QQYYSTPWT |
| | | SEQ ID NO: 721 | SEQ ID NO: 878 | SEQ ID NO: 1035 |
| iPS: 361268 | 5C2.014 | KSSQSVLSSSNNKNYIA | WASTRES | QQYYSTPWT |
| | | SEQ ID NO: 722 | SEQ ID NO: 879 | SEQ ID NO: 1036 |
| iPS: 359669 | 5C2.015 | KSSQSVLSSSNNKNYIA | WASTRES | QQYYSTPWT |
| | | SEQ ID NO: 723 | SEQ ID NO: 880 | SEQ ID NO: 1037 |
| iPS: 359678 | 5C2.016 | KSSQSVLSSSNNKNYIA | WASTRES | QQYYSTPWT |
| | | SEQ ID NO: 724 | SEQ ID NO: 881 | SEQ ID NO: 1038 |
| iPS: 361275 | 11C1.002 | KSSQSVLSSSNNKNYLA | WTSTRES | QQYYRTPWT |
| | | SEQ ID NO: 725 | SEQ ID NO: 882 | SEQ ID NO: 1039 |
| iPS: 361282 | 11C1.003 | KSSQSVLSSSNNKNYLA | WTSTRES | QQYYRTPWT |
| | | SEQ ID NO: 726 | SEQ ID NO: 883 | SEQ ID NO: 1040 |
| iPS: 361289 | 11C1.004 | KSSQSVLSSSNNKNYLA | WTSTRDS | QQYYRTPWT |
| | | SEQ ID NO: 727 | SEQ ID NO: 884 | SEQ ID NO: 1041 |
| iPS: 359712 | 11C1.005 | KSSQSVLSSSNNKNYLA | WTSTRES | QQYYRTPWT |
| | | SEQ ID NO: 728 | SEQ ID NO: 885 | SEQ ID NO: 1042 |
| iPS: 361293 | 11C1.006 | KSSQSVLSSSNNKNYLA | WTSTRES | QQYYRTPWT |
| | | SEQ ID NO: 729 | SEQ ID NO: 886 | SEQ ID NO: 1043 |
| iPS: 361297 | 11C1.007 | KSSQSVLSSSNNKNYLA | WTSTRDS | QQYYRTPWT |
| | | SEQ ID NO: 730 | SEQ ID NO: 887 | SEQ ID NO: 1044 |
| iPS: 361301 | 11C1.008 | KSSQSVLSSSNNKNYLA | WTSTRDS | QQYYRTPWT |
| | | SEQ ID NO: 731 | SEQ ID NO: 888 | SEQ ID NO: 1045 |
| iPS: 359703 | 11C1.009 | KSSQSVLSSSNNKNYLA | WTSTRES | QQYYRTPWT |
| | | SEQ ID NO: 732 | SEQ ID NO: 889 | SEQ ID NO: 1046 |
| iPS: 361305 | 11C1.010 | KSSQSVLSSSNNKNYLA | WTSTRES | QQYYRTPWT |
| | | SEQ ID NO: 733 | SEQ ID NO: 890 | SEQ ID NO: 1047 |
| iPS: 361312 | 13H12.002 | KSSQSVLSSSNNKNYIA | WASTRES | QQYYSTPWT |
| | | SEQ ID NO: 734 | SEQ ID NO: 891 | SEQ ID NO: 1048 |
| iPS: 359744 | 13H12.003 | KSSQSVLSSSNNKNYIA | WASTRES | QQYYSTPWT |
| | | SEQ ID NO: 735 | SEQ ID NO: 892 | SEQ ID NO: 1049 |
| iPS: 361319 | 13H12.004 | KSSQSVLSSSNNKNYIA | WASTRES | QQYYSTPWT |
| | | SEQ ID NO: 736 | SEQ ID NO: 893 | SEQ ID NO: 1050 |
| iPS: 359737 | 13H12.005 | KSSQSVLSSSNNKNYIA | WASTRES | QQYYSTPWT |
| | | SEQ ID NO: 737 | SEQ ID NO: 894 | SEQ ID NO: 1051 |
| iPS: 361326 | 13H12.006 | KSSQSVLSSSNNKNYIA | WASTRES | QQYYSTPWT |
| | | SEQ ID NO: 738 | SEQ ID NO: 895 | SEQ ID NO: 1052 |
| iPS: 361330 | 12H11.002 | KSSQSVLSSSNNKNYLA | WTSTRDS | QQYYRTPWT |
| | | SEQ ID NO: 739 | SEQ ID NO: 896 | SEQ ID NO: 1053 |
| iPS: 361337 | 12H11.003 | KSSQSVLSSSNNKNYLA | WTSTRDS | QQYYRTPWT |
| | | SEQ ID NO: 740 | SEQ ID NO: 897 | SEQ ID NO: 1054 |
| iPS: 361344 | 12H11.004 | KSSQSVLSSSNNKNYLA | WTSTRDS | QQYYRTPWT |
| | | SEQ ID NO: 741 | SEQ ID NO: 898 | SEQ ID NO: 1055 |
| iPS: 361351 | 12H11.005 | KSSQSVLSSSNNKNYLA | WTSTRDS | QQYYRTPWT |
| | | SEQ ID NO: 742 | SEQ ID NO: 899 | SEQ ID NO: 1056 |
| iPS: 361358 | 12H11.006 | KSSQSVLSSSNNKNYLA | WTSTRDS | QQYYRTPWT |
| | | SEQ ID NO: 743 | SEQ ID NO: 900 | SEQ ID NO: 1057 |
| iPS: 361365 | 12H11.007 | KSSQSVLSSSNNKNYLA | WTSTRDS | QQYYRTPWT |
| | | SEQ ID NO: 744 | SEQ ID NO: 901 | SEQ ID NO: 1058 |
| iPS: 361372 | 12H11.008 | KSSQSVLSSSNNKNYLA | WTSTRDS | QQYYRTPWT |
| | | SEQ ID NO: 745 | SEQ ID NO: 902 | SEQ ID NO: 1059 |
| iPS: 361379 | 12H11.009 | KSSQSVLSSSNNKNYLA | WTSTRES | QQYYRTPWT |
| | | SEQ ID NO: 746 | SEQ ID NO: 903 | SEQ ID NO: 1060 |
| iPS: 361383 | 12H11.010 | KSSQSVLSSSNNKNYLA | WTSTRES | QQYYRTPWT |
| | | SEQ ID NO: 747 | SEQ ID NO: 904 | SEQ ID NO: 1061 |
| iPS: 361387 | 12H11.011 | KSSQSVLSSSNNKNYLA | WTSTRES | QQYYRTPWT |
| | | SEQ ID NO: 748 | SEQ ID NO: 905 | SEQ ID NO: 1062 |
| iPS: 361391 | 12H11.012 | KSSQSVLSSSNNKNYLA | WTSTRES | QQYYRTPWT |
| | | SEQ ID NO: 749 | SEQ ID NO: 906 | SEQ ID NO: 1063 |
| iPS: 361395 | 12H11.013 | KSSQSVLSSSNNKNYLA | WTSTRDS | QQYYRTPWT |
| | | SEQ ID NO: 750 | SEQ ID NO: 907 | SEQ ID NO: 1064 |
| iPS: 361402 | 12H11.014 | KSSQSVLSSSNNKNYLA | WTSTRES | QQYYRTPWT |
| | | SEQ ID NO: 751 | SEQ ID NO: 908 | SEQ ID NO: 1065 |
| iPS: 361406 | 2C2.005.001 | SGSSSNIGSQTVN | TNNQRPS | ATFDESLSGPV |
| | | SEQ ID NO: 752 | SEQ ID NO: 909 | SEQ ID NO: 1066 |
| iPS: 361412 | 2C2.005.002 | SGSSSNIGSQTVN | TNNQRPS | ATFDESLQGPV |
| | | SEQ ID NO: 753 | SEQ ID NO: 910 | SEQ ID NO: 1067 |
| iPS: 361418 | 2C2.005.003 | SGSSSNIGSNYVN | TNNQRPS | ATFDESLSGPV |
| | | SEQ ID NO: 754 | SEQ ID NO: 911 | SEQ ID NO: 1068 |
| iPS: 361424 | 2C2.005.004 | SGSSSNIGSNYVN | TNNQRPS | ATFDESLQGPV |
| | | SEQ ID NO: 755 | SEQ ID NO: 912 | SEQ ID NO: 1069 |
| iPS: 361430 | 2C2.005.005 | SGSSSNIGSQTVN | TNNQRPS | ATFDDSLNGPV |
| | | SEQ ID NO: 756 | SEQ ID NO: 913 | SEQ ID NO: 1070 |
| iPS: 361436 | 2C2.005.006 | SGSSSNIGSNYVN | TNNQRPS | ATFDDSLNGPV |
| | | SEQ ID NO: 757 | SEQ ID NO: 914 | SEQ ID NO: 1071 |
| iPS: 361442 | 2C2.005.007 | SGSSSNIGSNTVN | TNNQRPS | ATFDESLNGPV |
| | | SEQ ID NO: 758 | SEQ ID NO: 915 | SEQ ID NO: 1072 |
| iPS: 361448 | 2C2.005.008 | SGSSSNIGSNTVN | TNNQRPS | ATFDDSLQGPV |
| | | SEQ ID NO: 759 | SEQ ID NO: 916 | SEQ ID NO: 1073 |
| iPS: 361454 | 2C2.005.009 | SGSSSNIGSNTVN | TNNQRPS | ATFDDSLSGPV |
| | | SEQ ID NO: 760 | SEQ ID NO: 917 | SEQ ID NO: 1074 |
| iPS: 361460 | 2C2.005.010 | SGSSSNIGSNTVN | TNNQRPS | ATFDDSLNAPV |
| | | SEQ ID NO: 761 | SEQ ID NO: 918 | SEQ ID NO: 1075 |
| iPS: 361466 | 2C2.005.011 | SGSSSNIGSNTVN | TNNQRPS | ATFDSSLNGPV |
| | | SEQ ID NO: 762 | SEQ ID NO: 919 | SEQ ID NO: 1076 |
| iPS: 361472 | 2C2.005.012 | SGSSSNIGSNYVN | TNNQRPS | ATFDSSLNAPV |
| | | SEQ ID NO: 763 | SEQ ID NO: 920 | SEQ ID NO: 1077 |
| iPS: 361478 | 2C2.005.013 | SGSSSNIGSNYVN | TNNQRPS | ATFDSSLNAPV |
| | | SEQ ID NO: 764 | SEQ ID NO: 921 | SEQ ID NO: 1078 |
| iPS: 361485 | 2C2.005.014 | SGSSSNIGSQTVN | TNNQRPS | ATFDESLSGPV |
| | | SEQ ID NO: 765 | SEQ ID NO: 922 | SEQ ID NO: 1079 |
| iPS: 361845 | 18F2.013 | RASQDLRNYLG | GASSLQS | LQHNNYPFT |
| | | SEQ ID NO: 766 | SEQ ID NO: 923 | SEQ ID NO: 1080 |
| iPS: 361851 | 6H1.009 | RASQDIRDYLG | GASSLQS | LQHNNYPFT |
| | | SEQ ID NO: 767 | SEQ ID NO: 924 | SEQ ID NO: 1081 |
| iPS: 361855 | 2C2.005.015 | SGSSSNIGSNTVN | TNNQRPS | ATFDSSLSGPV |
| | | SEQ ID NO: 768 | SEQ ID NO: 925 | SEQ ID NO: 1082 |
| iPS: 336067 | 5G12.006 | RASQTISRFLN | VASSLQS | QQSYSTLIS |
| | | SEQ ID NO: 769 | SEQ ID NO: 926 | SEQ ID NO: 1083 |
| iPS: 336169 | 17B11.002 | SGSSSNIGNNYVS | DNNKRPS | GTWDSSLSAVV |
| | | SEQ ID NO: 770 | SEQ ID NO: 927 | SEQ ID NO: 1084 |
| iPS: 361127 | 5G12.006.001 | RASQTISRFLN | VASSLQS | QQSYSTLIS |
| | | SEQ ID NO: 771 | SEQ ID NO: 928 | SEQ ID NO: 1085 |
| iPS: 361136 | 5G12.006.002 | RASQTISRFLN | VASSLQS | QQSYSTLIS |
| | | SEQ ID NO: 772 | SEQ ID NO: 929 | SEQ ID NO: 1086 |
| iPS: 361140 | 5G12.006.003 | RASQTISRFLN | VASSLQS | QQSYSTLIS |
| | | SEQ ID NO: 773 | SEQ ID NO: 930 | SEQ ID NO: 1087 |
| iPS: 359919 | 5G12.006.004 | RASQTISRFLN | VASSLQS | QQSYSTLIS |
| | | SEQ ID NO: 774 | SEQ ID NO: 931 | SEQ ID NO: 1088 |
| iPS: 361144 | 5G12.006.005 | RASQTISRFLN | VASSLQS | QQSYSTLIS |
| | | SEQ ID NO: 775 | SEQ ID NO: 932 | SEQ ID NO: 1089 |
| iPS: 361151 | 5G12.006.006 | RASQTISRFLN | VASSLQS | QQSYSTLIS |
| | | SEQ ID NO: 776 | SEQ ID NO: 933 | SEQ ID NO: 1090 |
| iPS: 361491 | 17B11.002.001 | SGSSSNIGNNYVS | DNNKRPS | GTWDSSLSAVV |
| | | SEQ ID NO: 777 | SEQ ID NO: 934 | SEQ ID NO: 1091 |
| iPS: 361499 | 17B11.002.002 | SGSSSNIGNNYVS | DNNKRPS | GTWDSSLSAVV |
| | | SEQ ID NO: 778 | SEQ ID NO: 935 | SEQ ID NO: 1092 |
| iPS: 361503 | 17B11.002.003 | SGSSSNIGNNYVS | DNNKRPS | GTWDSSLSAVV |
| | | SEQ ID NO: 779 | SEQ ID NO: 936 | SEQ ID NO: 1093 |
| iPS: 361507 | 17B11.002.004 | SGSSSNIGNNYVS | DNNKRPS | GTFESSLSAVV |
| | | SEQ ID NO: 780 | SEQ ID NO: 937 | SEQ ID NO: 1094 |
| iPS: 361514 | 17B11.002.005 | SGSSSNIGNNYVS | DNNKRPS | GTWDSSLSAVV |
| | | SEQ ID NO: 781 | SEQ ID NO: 938 | SEQ ID NO: 1095 |
| iPS: 360582 | 17B11.002.006 | SGSSSNIGNNYVS | DNNKRPS | GTWDSSLSAVV |
| | | SEQ ID NO: 782 | SEQ ID NO: 939 | SEQ ID NO: 1096 |
| iPS: 361518 | 17B11.002.007 | SGSSSNIGNNYVS | DNNKRPS | GTWDSSLSAVV |
| | | SEQ ID NO: 783 | SEQ ID NO: 940 | SEQ ID NO: 1097 |
| iPS: 360570 | 17B11.002.008 | SGSSSNIGNNYVS | DNNKRPS | GTFESSLSAVV |
| | | SEQ ID NO: 784 | SEQ ID NO: 941 | SEQ ID NO: 1098 |
| iPS: 362051 | 5G12.005.001 | RASQTISRFLN | VASSLQS | QQSYSTLLS |
| | | SEQ ID NO: 785 | SEQ ID NO: 942 | SEQ ID NO: 1099 |

TABLE 13
CDRH1, CDRH2, and CDRH3 Amino Acid Sequences of Example Anti-GIPR Antibodies

| Identifier | Construct | CDRH1 | CDRH2 | CDRH3 |
|---|---|---|---|---|
| iPS: 336175 | 2C2.005 | GYYMH | WINPNSGGTNYAQKFQG | GGDYVWGTYRPHYYYGMDV |
| | | SEQ ID NO: 1100 | SEQ ID NO: 1257 | SEQ ID NO: 1414 |
| iPS: 335914 | 4B1 | NFGMH | VIWYDGSNENYADSVKG | DGTIFGVVLGDY |
| | | SEQ ID NO: 1101 | SEQ ID NO: 1258 | SEQ ID NO: 1415 |
| iPS: 335938 | 6H1 | YFGMH | VIWYDGSNKYYADSVKG | DGTIFGVLLGDY |
| | | SEQ ID NO: 1102 | SEQ ID NO: 1259 | SEQ ID NO: 1416 |
| iPS: 335941 | 2F11 | SYGMH | VIWYDGSNKYYADSVKG | DRTIFGVVLDY |
| | | SEQ ID NO: 1103 | SEQ ID NO: 1260 | SEQ ID NO: 1417 |
| iPS: 335970 | 5C2 | GYYIH | WINPDSGGTDYSQRFQG | EATIFGMVIVPFDY |
| | | SEQ ID NO: 1104 | SEQ ID NO: 1261 | SEQ ID NO: 1418 |
| iPS: 335978 | 13H12 | GYYIH | WINPDSGGTDYSQRFQG | EATIFGMVIVPFDY |
| | | SEQ ID NO: 1105 | SEQ ID NO: 1262 | SEQ ID NO: 1419 |
| iPS: 335986 | 11C1 | GYYIH | WISPNSGDTSYAQKFQD | EATIFGMLIVPFDY |
| | | SEQ ID NO: 1106 | SEQ ID NO: 1263 | SEQ ID NO: 1420 |
| iPS: 335994 | 12H11 | GYYIH | WISPNNGDTNYAQKFQD | EATIFGMLIVPFDY |
| | | SEQ ID NO: 1107 | SEQ ID NO: 1264 | SEQ ID NO: 1421 |
| iPS: 336024 | 18E3 | SGGYYWS | YIYYSGSTYYNPSLKS | DRITIFGVVMGGGMDV |
| | | SEQ ID NO: 1108 | SEQ ID NO: 1265 | SEQ ID NO: 1422 |
| iPS: 336041 | 2G10_LC1 | NYGMH | AIWFDGSDKYYADSVKG | DQAIFGVVPDY |
| | | SEQ ID NO: 1109 | SEQ ID NO: 1266 | SEQ ID NO: 1423 |
| iPS: 336077 | 4H9.004 | YFGMH | VIWYDGSNKYYADSVKG | DGTIFGVLLGDY |
| | | SEQ ID NO: 1110 | SEQ ID NO: 1267 | SEQ ID NO: 1424 |
| iPS: 336088 | 6A5.004 | SYGMH | AIWYDGNNKYYADSVKG | DRTIFGVVLEY |
| | | SEQ ID NO: 1111 | SEQ ID NO: 1268 | SEQ ID NO: 1425 |
| iPS: 336099 | 17H11.004 | SYYWS | RIYTSGSTNYNPSLKS | DVAVAGFDY |
| | | SEQ ID NO: 1112 | SEQ ID NO: 1269 | SEQ ID NO: 1426 |
| iPS: 359621 | 17H11.004.001 | SYYWS | RIYTSGSTNYNPSLKS | DVAVAGFDY |
| | | SEQ ID NO: 1113 | SEQ ID NO: 1270 | SEQ ID NO: 1427 |
| iPS: 359781 | 4H9.004.001 | YFGMH | VIWYDASNKYYADAVKG | DGTIFGVLLGDY |
| | | SEQ ID NO: 1114 | SEQ ID NO: 1271 | SEQ ID NO: 1428 |
| iPS: 360929 | 4H9.004.002 | YFGMH | VIWYDASNKYYADSVKG | DGTIFGVLLGDY |
| | | SEQ ID NO: 1115 | SEQ ID NO: 1272 | SEQ ID NO: 1429 |
| iPS: 360936 | 4H9.004.003 | YFGMH | VIWYDGSNKYYADAVKG | DGTIFGVLLGDY |
| | | SEQ ID NO: 1116 | SEQ ID NO: 1273 | SEQ ID NO: 1430 |
| iPS: 360943 | 4H9.004.004 | YFGMH | VIWYDASNKYYADAVKG | DGTIFGVLLGDY |
| | | SEQ ID NO: 1117 | SEQ ID NO: 1274 | SEQ ID NO: 1431 |
| iPS: 360949 | 4H9.004.005 | YFGMH | VIWYDASNKYYADAVKG | DGTIFGVLLGDY |
| | | SEQ ID NO: 1118 | SEQ ID NO: 1275 | SEQ ID NO: 1432 |
| iPS: 359761 | 4H9.004.006 | YFGMH | VIWYDASNKYYADAVKG | DRTIFGVLLGDY |
| | | SEQ ID NO: 1119 | SEQ ID NO: 1276 | SEQ ID NO: 1433 |
| iPS: 360955 | 4B1.010 | NFGMH | VIWYDASNENYADAVKG | DGTIFGVVLGDY |
| | | SEQ ID NO: 1120 | SEQ ID NO: 1277 | SEQ ID NO: 1434 |
| iPS: 360962 | 4B1.011 | NFGMH | VIWYDASNENYADAVKG | DGTIFGVVLGDY |
| | | SEQ ID NO: 1121 | SEQ ID NO: 1278 | SEQ ID NO: 1435 |
| iPS: 360968 | 4B1.012 | NFGMH | VIWYDASNENYADAVKG | DGTIFGVVLGDY |
| | | SEQ ID NO: 1122 | SEQ ID NO: 1279 | SEQ ID NO: 1436 |
| iPS: 360974 | 4B1.013 | NFGMH | VIWYDASNENYADSVKG | DGTIFGVVLGDY |
| | | SEQ ID NO: 1123 | SEQ ID NO: 1280 | SEQ ID NO: 1437 |
| iPS: 360978 | 4B1.014 | NFGMH | VIWYDGSNENYADAVKG | DGTIFGVVLGDY |
| | | SEQ ID NO: 1124 | SEQ ID NO: 1281 | SEQ ID NO: 1438 |
| iPS: 359785 | 4B1.015 | NFGMH | VIWYDASNENYADAVKG | DRTIFGVVLGDY |
| | | SEQ ID NO: 1125 | SEQ ID NO: 1282 | SEQ ID NO: 1439 |
| iPS: 360982 | 4B1.016 | NFGMH | VIWYDASNENYADAVKG | DGTIFGVVLGDY |
| | | SEQ ID NO: 1126 | SEQ ID NO: 1283 | SEQ ID NO: 1440 |
| iPS: 335922 | 18F2 | NFGMH | VIWYDGSNENYADSVKG | DGTIFGVVLGDY |
| | | SEQ ID NO: 1127 | SEQ ID NO: 1284 | SEQ ID NO: 1441 |
| iPS: 360986 | 18F2.002 | NFGMH | VIWYDASNENYADAVKG | DGTIFGVVLGDY |
| | | SEQ ID NO: 1128 | SEQ ID NO: 1285 | SEQ ID NO: 1442 |
| iPS: 360995 | 18F2.003 | NFGMH | VIWYDASNENYADAVKG | DGTIFGVVLGDY |
| | | SEQ ID NO: 1129 | SEQ ID NO: 1286 | SEQ ID NO: 1443 |
| iPS: 360999 | 18F2.004 | NFGMH | VIWYDASNENYADAVKG | DGTIFGVVLGDY |
| | | SEQ ID NO: 1130 | SEQ ID NO: 1287 | SEQ ID NO: 1444 |
| iPS: 361005 | 18F2.005 | NFGMH | VIWYDASNENYADAVKG | DGTIFGVVLGDY |
| | | SEQ ID NO: 1131 | SEQ ID NO: 1288 | SEQ ID NO: 1445 |
| iPS: 361009 | 18F2.006 | NFGMH | VIWYDASNENYADAVKG | DGTIFGVVLGDY |
| | | SEQ ID NO: 1132 | SEQ ID NO: 1289 | SEQ ID NO: 1446 |
| iPS: 361013 | 18F2.007 | NFGMH | VIWYDASNENYADAVKG | DGTIFGVVLGDY |
| | | SEQ ID NO: 1133 | SEQ ID NO: 1290 | SEQ ID NO: 1447 |
| iPS: 361017 | 18F2.008 | NFGMH | VIWYDASNENYADAVKG | DGTIFGVVLGDY |
| | | SEQ ID NO: 1134 | SEQ ID NO: 1291 | SEQ ID NO: 1448 |
| iPS: 361021 | 18F2.009 | NFGMH | VIWYDGSNENYADAVKG | DGTIFGVVLGDY |
| | | SEQ ID NO: 1135 | SEQ ID NO: 1292 | SEQ ID NO: 1449 |
| iPS: 361028 | 18F2.010 | NFGMH | VIWYDASNENYADSVKG | DGTIFGVVLGDY |
| | | SEQ ID NO: 1136 | SEQ ID NO: 1293 | SEQ ID NO: 1450 |
| iPS: 360535 | 18F2.011 | NFGMH | VIWYDASNENYADAVKG | DRTIFGVVLGDY |
| | | SEQ ID NO: 1137 | SEQ ID NO: 1294 | SEQ ID NO: 1451 |
| iPS: 361035 | 18F2.012 | NFGMH | VIWYDASNENYADAVKG | DGTIFGVVLGDY |
| | | SEQ ID NO: 1138 | SEQ ID NO: 1295 | SEQ ID NO: 1452 |
| iPS: 359940 | 2F11.002 | SYGMH | VIWYDASNKYYADAVKG | DRTIFGVVLDY |
| | | SEQ ID NO: 1139 | SEQ ID NO: 1296 | SEQ ID NO: 1453 |
| iPS: 361039 | 2F11.003 | SYGMH | VIWYDASNKYYADAVKG | DRTIFGVVLDY |
| | | SEQ ID NO: 1140 | SEQ ID NO: 1297 | SEQ ID NO: 1454 |
| iPS: 361043 | 2F11.004 | SYGMH | VIWYDASNKYYADAVKG | DRTIFGVVLDY |
| | | SEQ ID NO: 1141 | SEQ ID NO: 1298 | SEQ ID NO: 1455 |
| iPS: 361049 | 2F11.005 | SYGMH | VIWYDASNKYYADAVKG | DRTIFGVVLDY |
| | | SEQ ID NO: 1142 | SEQ ID NO: 1299 | SEQ ID NO: 1456 |
| iPS: 361055 | 2F11.006 | SYGMH | VIWYDASNKYYADAVKG | DRTIFGVVLDY |
| | | SEQ ID NO: 1143 | SEQ ID NO: 1300 | SEQ ID NO: 1457 |
| iPS: 361059 | 2F11.007 | SYGMH | VIWYDASNKYYADAVKG | DRTIFGVVLDY |
| | | SEQ ID NO: 1144 | SEQ ID NO: 1301 | SEQ ID NO: 1458 |
| iPS: 361063 | 2F11.008 | SYGMH | VIWYDASNKYYADAVKG | DRTIFGVVLDY |
| | | SEQ ID NO: 1145 | SEQ ID NO: 1302 | SEQ ID NO: 1459 |
| iPS: 359949 | 2F11.009 | SYGMH | VIWYDASNKYYADSVKG | DRTIFGVVLDY |
| | | SEQ ID NO: 1146 | SEQ ID NO: 1303 | SEQ ID NO: 1460 |
| iPS: 359956 | 2F11.010 | SYGMH | VIWYDGSNKYYADAVKG | DRTIFGVVLDY |
| | | SEQ ID NO: 1147 | SEQ ID NO: 1304 | SEQ ID NO: 1461 |
| iPS: 359865 | 6H1.002 | YFGMH | VIWYDASNKYYADAVKG | DGTIFGVLLGDY |
| | | SEQ ID NO: 1148 | SEQ ID NO: 1305 | SEQ ID NO: 1462 |
| iPS: 359869 | 6H1.003 | YFGMH | VIWYDASNKYYADAVKG | DRTIFGVLLGDY |
| | | SEQ ID NO: 1149 | SEQ ID NO: 1306 | SEQ ID NO: 1463 |
| iPS: 359873 | 6H1.004 | YFGMH | VIWYDASNKYYADAVKG | DGTIFGVLLGDY |
| | | SEQ ID NO: 1150 | SEQ ID NO: 1307 | SEQ ID NO: 1464 |
| iPS: 359877 | 6H1.005 | YFGMH | VIWYDASNKYYADAVKG | DRTIFGVLLGDY |
| | | SEQ ID NO: 1151 | SEQ ID NO: 1308 | SEQ ID NO: 1465 |
| iPS: 361067 | 6H1.006 | YFGMH | VIWYDGSNKYYADSVKG | DGTIFGVLLGDY |
| | | SEQ ID NO: 1152 | SEQ ID NO: 1309 | SEQ ID NO: 1466 |
| iPS: 361071 | 6H1.007 | YFGMH | VIWYDASNKYYADSVKG | DGTIFGVLLGDY |
| | | SEQ ID NO: 1153 | SEQ ID NO: 1310 | SEQ ID NO: 1467 |
| iPS: 361075 | 6H1.008 | YFGMH | VIWYDGSNKYYADAVKG | DGTIFGVLLGDY |
| | | SEQ ID NO: 1154 | SEQ ID NO: 1311 | SEQ ID NO: 1468 |
| iPS: 361079 | 6A5.004.001 | SYGMH | AIWYDANNKYYADAVKG | DRTIFGVVLEY |
| | | SEQ ID NO: 1155 | SEQ ID NO: 1312 | SEQ ID NO: 1469 |
| iPS: 361085 | 6A5.004.002 | SYGMH | AIWYDANNKYYADAVKG | DRTIFGVVLEY |
| | | SEQ ID NO: 1156 | SEQ ID NO: 1313 | SEQ ID NO: 1470 |
| iPS: 361091 | 6A5.004.003 | SYGMH | AIWYDANNKYYADAVKG | DRTIFGVVLEY |
| | | SEQ ID NO: 1157 | SEQ ID NO: 1314 | SEQ ID NO: 1471 |
| iPS: 361095 | 6A5.004.004 | SYGMH | AIWYDANNKYYADAVKG | DRTIFGVVLEY |
| | | SEQ ID NO: 1158 | SEQ ID NO: 1315 | SEQ ID NO: 1472 |
| iPS: 361101 | 6A5.004.005 | SYGMH | AIWYDANNKYYADAVKG | DRTIFGVVLEY |
| | | SEQ ID NO: 1159 | SEQ ID NO: 1316 | SEQ ID NO: 1473 |
| iPS: 361105 | 6A5.004.006 | SYGMH | AIWYDANNKYYADAVKG | DRTIFGVVLEY |
| | | SEQ ID NO: 1160 | SEQ ID NO: 1317 | SEQ ID NO: 1474 |
| iPS: 361109 | 6A5.004.007 | SYGMH | AIWYDANNKYYADAVKG | DRTIFGVVLEY |
| | | SEQ ID NO: 1161 | SEQ ID NO: 1318 | SEQ ID NO: 1475 |
| iPS: 361113 | 6A5.004.008 | SYGMH | AIWYDGNNKYYADAVKG | DRTIFGVVLEY |
| | | SEQ ID NO: 1162 | SEQ ID NO: 1319 | SEQ ID NO: 1476 |
| iPS: 361120 | 6A5.004.009 | SYGMH | AIWYDANNKYYADSVKG | DRTIFGVVLEY |
| | | SEQ ID NO: 1163 | SEQ ID NO: 1320 | SEQ ID NO: 1477 |
| iPS: 359896 | 6A5.004.010 | SYGMH | AIWYDANNKYYADAVKG | DRTIFGVVLEY |
| | | SEQ ID NO: 1164 | SEQ ID NO: 1321 | SEQ ID NO: 1478 |
| iPS: 359890 | 6A5.004.011 | SYGMH | AIWYDANNKYYADAVKG | DRTIFGVVLEY |
| | | SEQ ID NO: 1165 | SEQ ID NO: 1322 | SEQ ID NO: 1479 |
| iPS: 359567 | 2A11.002 | SYGMH | VIWYDASNKYYEDAVKG | GITIFGHGFEY |
| | | SEQ ID NO: 1166 | SEQ ID NO: 1323 | SEQ ID NO: 1480 |
| iPS: 335965 | 2A11.003 | SYGMH | VIWYDGSNKYYEDSVKG | GITIFGHGFEY |
| | | SEQ ID NO: 1167 | SEQ ID NO: 1324 | SEQ ID NO: 1481 |
| iPS: 361158 | 2A11.004 | SYGMH | VIWYDGSNKYYEDAVKG | GITIFGHGFEY |
| | | SEQ ID NO: 1168 | SEQ ID NO: 1325 | SEQ ID NO: 1482 |
| iPS: 361165 | 2A11.005 | SYGMH | VIWYDASNKYYEDSVKG | GITIFGHGFEY |
| | | SEQ ID NO: 1169 | SEQ ID NO: 1326 | SEQ ID NO: 1483 |
| iPS: 361172 | 2G10_LC1.003 | NYGMH | AIWFDASDKYYADAVKG | DQAIFGVVPDY |
| | | SEQ ID NO: 1170 | SEQ ID NO: 1327 | SEQ ID NO: 1484 |
| iPS: 361178 | 2G10_LC1.004 | NYGMH | AIWFDASDKYYADSVKG | DQAIFGVVPDY |
| | | SEQ ID NO: 1171 | SEQ ID NO: 1328 | SEQ ID NO: 1485 |
| iPS: 361185 | 2G10_LC1.005 | NYGMH | AIWFDGSDKYYADAVKG | DQAIFGVVPDY |
| | | SEQ ID NO: 1172 | SEQ ID NO: 1329 | SEQ ID NO: 1486 |
| iPS: 361192 | 2G10_LC1.006 | NYGMH | AIWFDASDKYYADAVKG | DQAIFGVVPDY |
| | | SEQ ID NO: 1173 | SEQ ID NO: 1330 | SEQ ID NO: 1487 |
| iPS: 359609 | 2G10_LC1.007 | NYGMH | AIWFDASDKYYADAVKG | DQAIFGVVPDY |
| | | SEQ ID NO: 1174 | SEQ ID NO: 1331 | SEQ ID NO: 1488 |
| iPS: 359615 | 2G10_LC1.008 | NYGMH | AIWFDASDKYYADAVKG | DQAIFGVVPDY |
| | | SEQ ID NO: 1175 | SEQ ID NO: 1332 | SEQ ID NO: 1489 |
| iPS: 361196 | 2G10_LC1.009 | NYGMH | AIWFDASDKYYADAVKG | DQAIFGVVPDY |
| | | SEQ ID NO: 1176 | SEQ ID NO: 1333 | SEQ ID NO: 1490 |
| iPS: 361202 | 2G10_LC1.010 | NYGMH | AIWFDASDKYYADAVKG | DQAIFGVVPDY |
| | | SEQ ID NO: 1177 | SEQ ID NO: 1334 | SEQ ID NO: 1491 |
| iPS: 359644 | 18E3.002 | SGGYYWS | YIYYSGSTYYNPSLKS | DRITIFGVVMGGGMDV |
| | | SEQ ID NO: 1178 | SEQ ID NO: 1335 | SEQ ID NO: 1492 |
| iPS: 361206 | 18E3.003 | SGGYYWS | YIYYSGSTYYNPSLKS | DRITIFGVVMGGGMDV |
| | | SEQ ID NO: 1179 | SEQ ID NO: 1336 | SEQ ID NO: 1493 |
| iPS: 359628 | 18E3.004 | SGGYYWS | YIYYSGSTYYNPSLKS | DRITIFGVVMGGGMDV |
| | | SEQ ID NO: 1180 | SEQ ID NO: 1337 | SEQ ID NO: 1494 |
| iPS: 359637 | 18E3.005 | SGGYYWS | YIYYSGSTYYNPSLKS | DRITIFGVVMGGGMDV |
| | | SEQ ID NO: 1181 | SEQ ID NO: 1338 | SEQ ID NO: 1495 |
| iPS: 361210 | 18E3.006 | SGGYYWS | YIYYSGSTYYNPSLKS | DRITIFGVVMGGGMDV |
| | | SEQ ID NO: 1182 | SEQ ID NO: 1339 | SEQ ID NO: 1496 |
| iPS: 361214 | 18E3.007 | SGGYYWS | YIYYSGSTYYNPSLKS | DRITIFGVVMGGGMDV |
| | | SEQ ID NO: 1183 | SEQ ID NO: 1340 | SEQ ID NO: 1497 |
| iPS: 361218 | 18E3.008 | SGGYYWS | YIYYSGSTYYNPSLKS | DRITIFGVVMGGGMDV |
| | | SEQ ID NO: 1184 | SEQ ID NO: 1341 | SEQ ID NO: 1498 |
| iPS: 361222 | 5C2.006 | GYYIH | WINPESGGTDYSQRFQG | EATIFGMVIVPFDY |
| | | SEQ ID NO: 1185 | SEQ ID NO: 1342 | SEQ ID NO: 1499 |
| iPS: 361229 | 5C2.007 | GYYIH | WINPESGGTDYSQRFQG | EATIFGMVIVPFDY |
| | | SEQ ID NO: 1186 | SEQ ID NO: 1343 | SEQ ID NO: 1500 |
| iPS: 361236 | 5C2.008 | GYYIH | WINPDAGGTDYSQRFQG | EATIFGMVIVPFDY |
| | | SEQ ID NO: 1187 | SEQ ID NO: 1344 | SEQ ID NO: 1501 |
| iPS: 359685 | 5C2.009 | GYYIH | WINPDAGGTDYSQRFQG | EATIFGMVIVPFDY |
| | | SEQ ID NO: 1188 | SEQ ID NO: 1345 | SEQ ID NO: 1502 |
| iPS: 361240 | 5C2.010 | GYYIH | WINPDSGGTDYSQRFQG | EATIFGMVIVPFDY |
| | | SEQ ID NO: 1189 | SEQ ID NO: 1346 | SEQ ID NO: 1503 |
| iPS: 361247 | 5C2.011 | GYYIH | WINPDSGGTDYSQRFQG | EATIFGMVIVPFDY |
| | | SEQ ID NO: 1190 | SEQ ID NO: 1347 | SEQ ID NO: 1504 |
| iPS: 361254 | 5C2.012 | GYYIH | WINPDSGGTDYSQRFQG | EATIFGMVIVPFDY |
| | | SEQ ID NO: 1191 | SEQ ID NO: 1348 | SEQ ID NO: 1505 |
| iPS: 361261 | 5C2.013 | GYYIH | WINPDAGGTDYSQRFQG | EATIFGMVIVPFDY |
| | | SEQ ID NO: 1192 | SEQ ID NO: 1349 | SEQ ID NO: 1506 |
| iPS: 361268 | 5C2.014 | GYYIH | WINPDAGGTDYSQRFQG | EATIFGMVIVPFDY |
| | | SEQ ID NO: 1193 | SEQ ID NO: 1350 | SEQ ID NO: 1507 |
| iPS: 359669 | 5C2.015 | GYYIH | WINPDAGGTDYSQRFQG | EATIFGMVIVPFDY |
| | | SEQ ID NO: 1194 | SEQ ID NO: 1351 | SEQ ID NO: 1508 |
| iPS: 359678 | 5C2.016 | GYYIH | WINPESGGTDYSQRFQG | EATIFGMVIVPFDY |
| | | SEQ ID NO: 1195 | SEQ ID NO: 1352 | SEQ ID NO: 1509 |
| iPS: 361275 | 11C1.002 | GYYIH | WISPNSGETSYAQKFQD | EATIFGMLIVPFDY |
| | | SEQ ID NO: 1196 | SEQ ID NO: 1353 | SEQ ID NO: 1510 |
| iPS: 361282 | 11C1.003 | GYYIH | WISPNSGDTSYAQKFQD | EATIFGMLIVPFDY |
| | | SEQ ID NO: 1197 | SEQ ID NO: 1354 | SEQ ID NO: 1511 |
| iPS: 361289 | 11C1.004 | GYYIH | WISPNSGETSYAQKFQD | EATIFGMLIVPFDY |
| | | SEQ ID NO: 1198 | SEQ ID NO: 1355 | SEQ ID NO: 1512 |
| iPS: 359712 | 11C1.005 | GYYIH | WISPNSGETSYAQKFQD | EATIFGMLIVPFDY |
| | | SEQ ID NO: 1199 | SEQ ID NO: 1356 | SEQ ID NO: 1513 |
| iPS: 361293 | 11C1.006 | GYYIH | WISPNSGDTSYAQKFQD | EATIFGMLIVPFDY |
| | | SEQ ID NO: 1200 | SEQ ID NO: 1357 | SEQ ID NO: 1514 |
| iPS: 361297 | 11C1.007 | GYYIH | WISPNSGETSYAQKFQD | EATIFGMLIVPFDY |
| | | SEQ ID NO: 1201 | SEQ ID NO: 1358 | SEQ ID NO: 1515 |
| iPS: 361301 | 11C1.008 | GYYIH | WISPNSGDTSYAQKFQD | EATIFGMLIVPFDY |
| | | SEQ ID NO: 1202 | SEQ ID NO: 1359 | SEQ ID NO: 1516 |
| iPS: 359703 | 11C1.009 | GYYIH | WISPNSGETSYAQKFQD | EATIFGMLIVPFDY |
| | | SEQ ID NO: 1203 | SEQ ID NO: 1360 | SEQ ID NO: 1517 |
| iPS: 361305 | 11C1.010 | GYYIH | WISPNSGETSYAQKFQD | EATIFGMLIVPFDY |
| | | SEQ ID NO: 1204 | SEQ ID NO: 1361 | SEQ ID NO: 1518 |
| iPS: 361312 | 13H12.002 | GYYIH | WINPESGGTDYSQRFQG | EATIFGMVIVPFDY |
| | | SEQ ID NO: 1205 | SEQ ID NO: 1362 | SEQ ID NO: 1519 |
| iPS: 359744 | 13H12.003 | GYYIH | WINPESGGTDYSQRFQG | EATIFGMVIVPFDY |
| | | SEQ ID NO: 1206 | SEQ ID NO: 1363 | SEQ ID NO: 1520 |
| iPS: 361319 | 13H12.004 | GYYIH | WINPDSGGTDYSQRFQG | EATIFGMVIVPFDY |
| | | SEQ ID NO: 1207 | SEQ ID NO: 1364 | SEQ ID NO: 1521 |
| iPS: 359737 | 13H12.005 | GYYIH | WINPESGGTDYSQRFQG | EATIFGMVIVPFDY |
| | | SEQ ID NO: 1208 | SEQ ID NO: 1365 | SEQ ID NO: 1522 |
| iPS: 361326 | 13H12.006 | GYYIH | WINPESGGTDYSQRFQG | EATIFGMVIVPFDY |
| | | SEQ ID NO: 1209 | SEQ ID NO: 1366 | SEQ ID NO: 1523 |
| iPS: 361330 | 12H11.002 | GYYIH | WISPNQGETNYAQKFQD | EATIFGMLIVPFDY |
| | | SEQ ID NO: 1210 | SEQ ID NO: 1367 | SEQ ID NO: 1524 |
| iPS: 361337 | 12H11.003 | GYYIH | WISPNQGDTNYAQKFQD | EATIFGMLIVPFDY |
| | | SEQ ID NO: 1211 | SEQ ID NO: 1368 | SEQ ID NO: 1525 |
| iPS: 361344 | 12H11.004 | GYYIH | WISPNQGETNYAQKFQD | EATIFGMLIVPFDY |
| | | SEQ ID NO: 1212 | SEQ ID NO: 1369 | SEQ ID NO: 1526 |
| iPS: 361351 | 12H11.005 | GYYIH | WISPNNGETNYAQKFQD | EATIFGMLIVPFDY |
| | | SEQ ID NO: 1213 | SEQ ID NO: 1370 | SEQ ID NO: 1527 |
| iPS: 361358 | 12H11.006 | GYYIH | WISPNNGDTNYAQKFQD | EATIFGMLIVPFDY |
| | | SEQ ID NO: 1214 | SEQ ID NO: 1371 | SEQ ID NO: 1528 |
| iPS: 361365 | 12H11.007 | GYYIH | WISPNQGDTNYAQKFQD | EATIFGMLIVPFDY |
| | | SEQ ID NO: 1215 | SEQ ID NO: 1372 | SEQ ID NO: 1529 |
| iPS: 361372 | 12H11.008 | GYYIH | WISPNNGETNYAQKFQD | EATIFGMLIVPFDY |
| | | SEQ ID NO: 1216 | SEQ ID NO: 1373 | SEQ ID NO: 1530 |
| iPS: 361379 | 12H11.009 | GYYIH | WISPNQGETNYAQKFQD | EATIFGMLIVPFDY |
| | | SEQ ID NO: 1217 | SEQ ID NO: 1374 | SEQ ID NO: 1531 |
| iPS: 361383 | 12H11.010 | GYYIH | WISPNNGDTNYAQKFQD | EATIFGMLIVPFDY |
| | | SEQ ID NO: 1218 | SEQ ID NO: 1375 | SEQ ID NO: 1532 |
| iPS: 361387 | 12H11.011 | GYYIH | WISPNQGDTNYAQKFQD | EATIFGMLIVPFDY |
| | | SEQ ID NO: 1219 | SEQ ID NO: 1376 | SEQ ID NO: 1533 |
| iPS: 361391 | 12H11.012 | GYYIH | WISPNNGETNYAQKFQD | EATIFGMLIVPFDY |
| | | SEQ ID NO: 1220 | SEQ ID NO: 1377 | SEQ ID NO: 1534 |
| iPS: 361395 | 12H11.013 | GYYIH | WISPNQGETNYAQKFQD | EATIFGMLIVPFDY |
| | | SEQ ID NO: 1221 | SEQ ID NO: 1378 | SEQ ID NO: 1535 |
| iPS: 361402 | 12H11.014 | GYYIH | WISPNQGETNYAQKFQD | EATIFGMLIVPFDY |
| | | SEQ ID NO: 1222 | SEQ ID NO: 1379 | SEQ ID NO: 1536 |
| iPS: 361406 | 2C2.005.001 | GYYMH | WINPNSGGTNYAQKFQG | GGDYVWGTYRPHYYYGMDV |
| | | SEQ ID NO: 1223 | SEQ ID NO: 1380 | SEQ ID NO: 1537 |
| iPS: 361412 | 2C2.005.002 | GYYMH | WINPNSGGTNYAQKFQG | GGDYVWGTYRPHYYYGMDV |
| | | SEQ ID NO: 1224 | SEQ ID NO: 1381 | SEQ ID NO: 1538 |
| iPS: 361418 | 2C2.005.003 | GYYMH | WINPNSGGTNYAQKFQG | GGDYVWGTYRPHYYYGMDV |
| | | SEQ ID NO: 1225 | SEQ ID NO: 1382 | SEQ ID NO: 1539 |
| iPS: 361424 | 2C2.005.004 | GYYMH | WINPNSGGTNYAQKFQG | GGDYVWGTYRPHYYYGMDV |
| | | SEQ ID NO: 1226 | SEQ ID NO: 1383 | SEQ ID NO: 1540 |
| iPS: 361430 | 2C2.005.005 | GYYMH | WINPNSGGTNYAQKFQG | GGDYVWGTYRPHYYYGMDV |
| | | SEQ ID NO: 1227 | SEQ ID NO: 1384 | SEQ ID NO: 1541 |
| iPS: 361436 | 2C2.005.006 | GYYMH | WINPNSGGTNYAQKFQG | GGDYVWGTYRPHYYYGMDV |
| | | SEQ ID NO: 1228 | SEQ ID NO: 1385 | SEQ ID NO: 1542 |
| iPS: 361442 | 2C2.005.007 | GYYMH | WINPNSGGTNYAQKFQG | GGDYVWGTYRPHYYYGMDV |
| | | SEQ ID NO: 1229 | SEQ ID NO: 1386 | SEQ ID NO: 1543 |
| iPS: 361448 | 2C2.005.008 | GYYMH | WINPNSGGTNYAQKFQG | GGDYVWGTYRPHYYYGMDV |
| | | SEQ ID NO: 1230 | SEQ ID NO: 1387 | SEQ ID NO: 1544 |
| iPS: 361454 | 2C2.005.009 | GYYMH | WINPNSGGTNYAQKFQG | GGDYVWGTYRPHYYYGMDV |
| | | SEQ ID NO: 1231 | SEQ ID NO: 1388 | SEQ ID NO: 1545 |
| iPS: 361460 | 2C2.005.010 | GYYMH | WINPNSGGTNYAQKFQG | GGDYVWGTYRPHYYYGMDV |
| | | SEQ ID NO: 1232 | SEQ ID NO: 1389 | SEQ ID NO: 1546 |
| iPS: 361466 | 2C2.005.011 | GYYMH | WINPNSGGTNYAQKFQG | GGDYVWGTYRPHYYYGMDV |
| | | SEQ ID NO: 1233 | SEQ ID NO: 1390 | SEQ ID NO: 1547 |
| iPS: 361472 | 2C2.005.012 | GYYMH | WINPNSGGTNYAQKFQG | GGDYVWGTYRPHYYYGMDV |
| | | SEQ ID NO: 1234 | SEQ ID NO: 1391 | SEQ ID NO: 1548 |
| iPS: 361478 | 2C2.005.013 | GYYMH | WINPNSGGTNYAQKFQG | GGDYVFGTYRPHYYYGMDV |
| | | SEQ ID NO: 1235 | SEQ ID NO: 1392 | SEQ ID NO: 1549 |
| iPS: 361485 | 2C2.005.014 | GYYMH | WINPNSGGTNYAQKFQG | GGDYVFGTYRPHYYYGMDV |
| | | SEQ ID NO: 1236 | SEQ ID NO: 1393 | SEQ ID NO: 1550 |
| iPS: 361845 | 18F2.013 | NFGMH | VIWYDASNENYADAVKG | DRTIFGVVLGDY |
| | | SEQ ID NO: 1237 | SEQ ID NO: 1394 | SEQ ID NO: 1551 |
| iPS: 361851 | 6H1.009 | YFGMH | VIWYDASNKYYADAVKG | DRTIFGVLLGDY |
| | | SEQ ID NO: 1238 | SEQ ID NO: 1395 | SEQ ID NO: 1552 |
| iPS: 361855 | 2C2.005.015 | GYYMH | WINPNSGGTNYAQKFQG | GGDYVWGTYRPHYYYGMDV |
| | | SEQ ID NO: 1239 | SEQ ID NO: 1396 | SEQ ID NO: 1553 |
| iPS: 336067 | 5G12.006 | SYGIH | VIWYDGSNKFHADSVKG | GKVAGMPEAFEI |
| | | SEQ ID NO: 1240 | SEQ ID NO: 1397 | SEQ ID NO: 1554 |
| iPS: 336169 | 17B11.002 | SYDIN | WMNPNSGNTGYAQKFQG | GGDYVWGSYRPYYYYYGMDV |
| | | SEQ ID NO: 1241 | SEQ ID NO: 1398 | SEQ ID NO: 1555 |
| iPS: 361127 | 5G12.006.001 | SYGIH | VIWYDASNKFHADAVKG | GKVAGMPEAFEI |
| | | SEQ ID NO: 1242 | SEQ ID NO: 1399 | SEQ ID NO: 1556 |
| iPS: 361136 | 5G12.006.002 | SYGIH | VIWYDASNKFHADAVKG | GKVAGMPEAFEI |
| | | SEQ ID NO: 1243 | SEQ ID NO: 1400 | SEQ ID NO: 1557 |
| iPS: 361140 | 5G12.006.003 | SYGIH | VIWYDASNKFHADAVKG | GKVAGMPEAFEI |
| | | SEQ ID NO: 1244 | SEQ ID NO: 1401 | SEQ ID NO: 1558 |
| iPS: 359919 | 5G12.006.004 | SYGIH | VIWYDASNKFHADAVKG | GKVAGMPEAFEI |
| | | SEQ ID NO: 1245 | SEQ ID NO: 1402 | SEQ ID NO: 1559 |
| iPS: 361144 | 5G12.006.005 | SYGIH | VIWYDASNKFHADSVKG | GKVAGMPEAFEI |
| | | SEQ ID NO: 1246 | SEQ ID NO: 1403 | SEQ ID NO: 1560 |
| iPS: 361151 | 5G12.006.006 | SYGIH | VIWYDGSNKFHADAVKG | GKVAGMPEAFEI |
| | | SEQ ID NO: 1247 | SEQ ID NO: 1404 | SEQ ID NO: 1561 |
| iPS: 361491 | 17B11.002.001 | SYDIN | WMNPNSGGTGYAQKFQG | GGDYVWGSYRPYYYYYGMDV |
| | | SEQ ID NO: 1248 | SEQ ID NO: 1405 | SEQ ID NO: 1562 |
| iPS: 361499 | 17B11.002.002 | SYDIN | WMNPNSGNTGYAQKFQG | GGDYVWGSYRPYYYYYGMDV |
| | | SEQ ID NO: 1249 | SEQ ID NO: 1406 | SEQ ID NO: 1563 |
| iPS: 361503 | 17B11.002.003 | SYDIN | WMNPNSGGTGYAQKFQG | GGDYVWGSYRPYYYYYGMDV |
| | | SEQ ID NO: 1250 | SEQ ID NO: 1407 | SEQ ID NO: 1564 |
| iPS: 361507 | 17B11.002.004 | SYDIN | WMNPNSGGTGYAQKFQG | GGDYVWGSYRPYYYYYGMDV |
| | | SEQ ID NO: 1251 | SEQ ID NO: 1408 | SEQ ID NO: 1565 |
| iPS: 361514 | 17B11.002.005 | SYDIN | WMNPNSGGTGYAQKFQG | GGDYVWGSYRPYYYYYGMDV |
| | | SEQ ID NO: 1252 | SEQ ID NO: 1409 | SEQ ID NO: 1566 |
| iPS: 360582 | 17B11.002.006 | SYDIN | WMNPNSGNTGYAQKFQG | GGDYVWGSYRPYYYYYGMDV |
| | | SEQ ID NO: 1253 | SEQ ID NO: 1410 | SEQ ID NO: 1567 |
| iPS: 361518 | 17B11.002.007 | SYDIN | WMNPNSGGTGYAQKFQG | GGDYVFGSYRPYYYYYGMDV |
| | | SEQ ID NO: 1254 | SEQ ID NO: 1411 | SEQ ID NO: 1568 |
| iPS: 360570 | 17B11.002.008 | SYDIN | WMNPNSGGTGYAQKFQG | GGDYVFGSYRPYYYYYGMDV |
| | | SEQ ID NO: 1255 | SEQ ID NO: 1412 | SEQ ID NO: 1569 |
| iPS: 362051 | 5G12.005.001 | SYGIH | VIWYDASNKFHADAVKG | GKVAGMPEAFEI |
| | | SEQ ID NO: 1256 | SEQ ID NO: 1413 | SEQ ID NO: 1570 |

TABLE 14
Light Chain (LC) Amino Acid Sequences of Example Anti-GIPR Antibodies

| | | | SEQ |
|---|---|---|---|
| Identifier | Construct | LC | ID NO: |
| iPS:336175 | 2C2.005 | QSVLTQPPSASGTPGQRVTISCSGSSSNIGSNTVNWYQHLPGTAPKLLIYTNNQRPS | 315 |
| | | GVPDRFSGSKSGTSASLAISGLQSEDEADYFCATFDDSLNGPVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:335914 | 4B1 | DIQMTQSPSSLSASIGDRVTITCRASQDIRDYLGWYQQKPGKAPKRLIYGASSLQSG | 316 |
| | | VPSRFSGSGSGTEFTLTISSLQPEDFATYYCLQHNNYPFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:335938 | 6H1 | DIQMTQSPSSLSASIGDRVTITCRASQDIRDYLGWYQQKPGKAPKRLIYGASSLQSG | 317 |
| | | VPSRFSGSGSGTEFTLTISSLQPEDFATYYCLQHNNYPFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:335941 | 2F11 | DIQMTQSPSSLSASVGDRVTITCQASQDISNYLNWYQQKPGKAPKLLIYDASNLET | 318 |
| | | GVPSRFSGSGSGTDFSFTISSLQPEDIATYYCQQYDILLTFGGGTKVEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:335970 | 5C2 | DIVMTQSPDSLAVSLGERATINCKSSQSVLSSSNNKNYIAWYQQQPGHPPKLLIYW | 319 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:335978 | 13H12 | DIVMTQSPDSLAVSLGERATINCKSSQSVLSSSNNKNYIAWYQQKPGHPPKLLIYW | 320 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:335986 | 11C1 | DIVMTQSPDSLSVSLGERATINCKSSQSVLSSSNNKNYLAWYQQKPGQPPNLLIYW | 321 |
| | | TSTRDSGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:335994 | 12H11 | DIVMTQSPDSLSVSLGERATINCKSSQSVLSSSNNKNYLAWYQQKPGQPPNLLIYW | 322 |
| | | TSTRDSGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:336024 | 18E3 | EIVLTQSPGTLSLSPGERATLSCRASQSISYSYLAWYQQKPGQAPRLLIYGASSRAT | 323 |
| | | GIPDRFSGSGSGTDFTLTISRQEPDDFAVYYCQQYGSSPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:336041 | 2G10_LC1 | EIVMTQSPATLSVSPGERATLSCRASQSVSSNLAWYQQKPGQAPRLLIYGAATRAT | 324 |
| | | GIPARVSGSGSGTEFTLTISSLQSEDFAVYYCQQYNNWPLTFGGGTKVEIKRTVAA | |
| | | PSVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:336077 | 4H9.004 | DIQMTQSPSSLSASVGDRVTITCRASQGIRNELGWYQQKPGKAPKRLIYGASSLQS | 325 |
| | | GVPSRFSGSGSGTEFTLTISSLQPEDFVTYYCLQHNSYPFTFGPGTKVDVKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:336088 | 6A5.004 | DIQMTQSPSSLSASVGDRVTITCQASQDITNYLNWYQQKPGKAPKLLIYDASNLEP | 326 |
| | | GVPSRFSGSGSGTDFTFTISSLQPEDIATYYCQQYDDLFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:336099 | 17H11.004 | DIQMTQSPSSVSASVGDRVTITCRASQGLIIWLAWYQQKPGKAPKLLIYAASSLQS | 327 |
| | | GVPSRFSGSGSGTDFTLTISSLQPEDFATYYCQQTNSFPPTFGQGTKVEIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:359621 | 17H11.004.001 | DIQMTQSPSSVSASVGDRVTITCRASQGLIIWLAWYQQKPGKAPKLLIYAASSLQS | 328 |
| | | GVPSRFSGSGSGTDFTLTISSLQPEDFATYYCQQTNSFPPTFGQGTKVEIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:359781 | 4H9.004.001 | DIQMTQSPSSLSASVGDRVTITCRASQGIRNELGWYQQKPGKAPKLLIYGASSLQS | 329 |
| | | GVPSRFSGSGSGTEFTLTISSLQPEDFVTYYCLQHNSYPFTFGQGTKVDVKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:360929 | 4H9.004.002 | DIQMTQSPSSLSASVGDRVTITCRASQGIRNELGWYQQKPGKAPKLLIYGASSLQS | 330 |
| | | GVPSRFSGSGSGTEFTLTISSLQPEDFVTYYCLQHNSYPFTFGQGTKVDVKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:360936 | 4H9.004.003 | DIQMTQSPSSLSASVGDRVTITCRASQGIRNELGWYQQKPGKAPKLLIYGASSLQS | 331 |
| | | GVPSRFSGSGSGTEFTLTISSLQPEDFVTYYCLQHNSYPFTFGQGTKVDVKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:360943 | 4H9.004.004 | DIQMTQSPSSLSASVGDRVTITCRASQGIRNELGWYQQKPGKAPKLLIYGASSLQS | 332 |
| | | GVPSRFSGSGSGTEFTLTISSLQPEDFVTYYCLQHNSYPFTFGPGTKVDVKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:360949 | 4H9.004.005 | DIQMTQSPSSLSASVGDRVTITCRASQGIRNELGWYQQKPGKAPKRLIYGASSLQS | 333 |
| | | GVPSRFSGSGSGTEFTLTISSLQPEDFVTYYCLQHNSYPFTFGQGTKVDVKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:359761 | 4H9.004.006 | DIQMTQSPSSLSASVGDRVTITCRASQGIRNELGWYQQKPGKAPKLLIYGASSLQS | 334 |
| | | GVPSRFSGSGSGTEFTLTISSLQPEDFVTYYCLQHNSYPFTFGQGTKVDVKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:360955 | 4B1.010 | DIQMTQSPSSLSASIGDRVTITCRASQDIRDYLGWYQQKPGKAPKLLIYGASSLQSG | 335 |
| | | VPSRFSGSGSGTEFTLTISSLQPEDFATYYCLQHNNYPFTFGQGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:360962 | 4B1.011 | DIQMTQSPSSLSASIGDRVTITCRASQDIRDYLGWYQQKPGKAPKRLIYGASSLQSG | 336 |
| | | VPSRFSGSGSGTEFTLTISSLQPEDFATYYCLQHNNYPFTFGQGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:360968 | 4B1.012 | DIQMTQSPSSLSASIGDRVTITCRASQDIRDYLGWYQQKPGKAPKLLIYGASSLQSG | 337 |
| | | VPSRFSGSGSGTEFTLTISSLQPEDFATYYCLQHNNYPFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:360974 | 4B1.013 | DIQMTQSPSSLSASIGDRVTITCRASQDIRDYLGWYQQKPGKAPKLLIYGASSLQSG | 338 |
| | | VPSRFSGSGSGTEFTLTISSLQPEDFATYYCLQHNNYPFTFGQGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:360978 | 4B1.014 | DIQMTQSPSSLSASIGDRVTITCRASQDIRDYLGWYQQKPGKAPKLLIYGASSLQSG | 339 |
| | | VPSRFSGSGSGTEFTLTISSLQPEDFATYYCLQHNNYPFTFGQGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:359785 | 4B1.015 | DIQMTQSPSSLSASIGDRVTITCRASQDIRDYLGWYQQKPGKAPKLLIYGASSLQSG | 340 |
| | | VPSRFSGSGSGTEFTLTISSLQPEDFATYYCLQHNNYPFTFGQGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:360982 | 4B1.016 | DIQMTQSPSSLSASIGDRVTITCRASQDIRDYLGWYQQKPGKAPKLLIYGASSLQSG | 341 |
| | | VPSRFSGSGSGTEFTLTISSLQPEDFATYYCLQHNNYPFTFGQGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:335922 | 18F2 | DIQMTQSPSSLSASIGDRVTITCRASQDLRNYLGWYHQKPGKAPKRLIYGASSLQS | 342 |
| | | GVPSRFSGSGSGTEFTLTISSLQPEDFATYYCLQHNNYPFTFGPGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:360986 | 18F2.002 | DIQMTQSPSSLSASIGDRVTITCRASQDLRNYLGWYQQKPGKAPKLLIYGASSLQS | 343 |
| | | GVPSRFSGSGSGTEFTLTISSLQPEDFATYYCLQHNNYPFTFGPGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:360995 | 18F2.003 | DIQMTQSPSSLSASIGDRVTITCRASQDLRNYLGWYQQKPGKAPKLLIYGASSLQS | 344 |
| | | GVPSRFSGSGSGTEFTLTISSLQPEDFATYYCLQHNNYPFTFGPGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:360999 | 18F2.004 | DIQMTQSPSSLSASIGDRVTITCRASQDLRNYLGWYHQKPGKAPKLLIYGASSLQS | 345 |
| | | GVPSRFSGSGSGTEFTLTISSLQPEDFATYYCLQHNNYPFTFGPGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361005 | 18F2.005 | DIQMTQSPSSLSASIGDRVTITCRASQDLRNYLGWYQQKPGKAPKRLIYGASSLQS | 346 |
| | | GVPSRFSGSGSGTEFTLTISSLQPEDFATYYCLQHNNYPFTFGPGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361009 | 18F2.006 | DIQMTQSPSSLSASIGDRVTITCRASQDLRNYLGWYQQKPGKAPKRLIYGASSLQS | 347 |
| | | GVPSRFSGSGSGTEFTLTISSLQPEDFATYYCLQHNNYPFTFGPGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361013 | 18F2.007 | DIQMTQSPSSLSASIGDRVTITCRASQDLRNYLGWYHQKPGKAPKLLIYGASSLQS | 348 |
| | | GVPSRFSGSGSGTEFTLTISSLQPEDFATYYCLQHNNYPFTFGPGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361017 | 18F2.008 | DIQMTQSPSSLSASIGDRVTITCRASQDLRNYLGWYHQKPGKAPKRLIYGASSLQS | 349 |
| | | GVPSRFSGSGSGTEFTLTISSLQPEDFATYYCLQHNNYPFTFGPGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361021 | 18F2.009 | DIQMTQSPSSLSASIGDRVTITCRASQDLRNYLGWYQQKPGKAPKLLIYGASSLQS | 350 |
| | | GVPSRFSGSGSGTEFTLTISSLQPEDFATYYCLQHNNYPFTFGPGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361028 | 18F2.010 | DIQMTQSPSSLSASIGDRVTITCRASQDLRNYLGWYQQKPGKAPKLLIYGASSLQS | 351 |
| | | GVPSRFSGSGSGTEFTLTISSLQPEDFATYYCLQHNNYPFTFGPGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:360535 | 18F2.011 | DIQMTQSPSSLSASIGDRVTITCRASQDLRNYLGWYQQKPGKAPKLLIYGASSLQS | 352 |
| | | GVPSRFSGSGSGTEFTLTISSLQPEDFATYYCLQHNNYPFTFGQGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361035 | 18F2.012 | DIQMTQSPSSLSASIGDRVTITCRASQDLRNYLGWYQQKPGKAPKLLIYGASSLQS | 353 |
| | | GVPSRFSGSGSGTEFTLTISSLQPEDFATYYCLQHNNYPFTFGPGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:359940 | 2F11.002 | DIQMTQSPSSLSASVGDRVTITCQASQDISNYLNWYQQKPGKAPKLLIYDASNLET | 354 |
| | | GVPSRFSGSGSGTDFSLTISSLQPEDFATYYCQQYDILLTFGGGTKVEIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361039 | 2F11.003 | DIQMTQSPSSLSASVGDRVTITCQASQDISNYLNWYQQKPGKAPKLLIYDASNLET | 355 |
| | | GVPSRFSGSGSGTDFSLTISSLQPEDFATYYCQQYDILLTFGGGTKVEIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361043 | 2F11.004 | DIQMTQSPSSLSASVGDRVTITCQASQDISNYLNWYQQKPGKAPKLLIYDASNLET | 356 |
| | | GVPSRFSGSGSGTDFSLTISSLQPEDIATYYCQQYDILLTFGGGTKVEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:361049 | 2F11.005 | DIQMTQSPSSLSASVGDRVTITCQASQDISNYLNWYQQKPGKAPKLLIYDASNLET | 357 |
| | | GVPSRFSGSGSGTDFSFTISSLQPEDFATYYCQQYDILLTFGGGTKVEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:361055 | 2F11.006 | DIQMTQSPSSLSASVGDRVTITCQASQDISNYLNWYQQKPGKAPKLLIYDASNLET | 358 |
| | | GVPSRFSGSGSGTDFSLTISSLQPEDIATYYCQQYDILLTFGGGTKVEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:361059 | 2F11.007 | DIQMTQSPSSLSASVGDRVTITCQASQDISNYLNWYQQKPGKAPKLLIYDASNLET | 359 |
| | | GVPSRFSGSGSGTDFSFTISSLQPEDFATYYCQQYDILLTFGGGTKVEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:361063 | 2F11.008 | DIQMTQSPSSLSASVGDRVTITCQASQDISNYLNWYQQKPGKAPKLLIYDASNLET | 360 |
| | | GVPSRFSGSGSGTDFSFTISSLQPEDIATYYCQQYDILLTFGGGTKVEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:359949 | 2F11.009 | DIQMTQSPSSLSASVGDRVTITCQASQDISNYLNWYQQKPGKAPKLLIYDASNLET | 361 |
| | | GVPSRFSGSGSGTDFSLTISSLQPEDFATYYCQQYDILLTFGGGTKVEIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:359956 | 2F11.010 | DIQMTQSPSSLSASVGDRVTITCQASQDISNYLNWYQQKPGKAPKLLIYDASNLET | 362 |
| | | GVPSRFSGSGSGTDFSLTISSLQPEDFATYYCQQYDILLTFGGGTKVEIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:359865 | 6H1.002 | DIQMTQSPSSLSASIGDRVTITCRASQDIRDYLGWYQQKPGKAPKRLIYGASSLQSG | 363 |
| | | VPSRFSGSGSGTEFTLTISSLQPEDFATYYCLQHNNYPFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:359869 | 6H1.003 | DIQMTQSPSSLSASIGDRVTITCRASQDIRDYLGWYQQKPGKAPKRLIYGASSLQSG | 364 |
| | | VPSRFSGSGSGTEFTLTISSLQPEDFATYYCLQHNNYPFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:359873 | 6H1.004 | DIQMTQSPSSLSASIGDRVTITCRASQDIRDYLGWYQQKPGKAPKLLIYGASSLQSG | 365 |
| | | VPSRFSGSGSGTEFTLTISSLQPEDFATYYCLQHNNYPFTFGQGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:359877 | 6H1.005 | DIQMTQSPSSLSASIGDRVTITCRASQDIRDYLGWYQQKPGKAPKLLIYGASSLQSG | 366 |
| | | VPSRFSGSGSGTEFTLTISSLQPEDFATYYCLQHNNYPFTFGQGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361067 | 6H1.006 | DIQMTQSPSSLSASIGDRVTITCRASQDIRDYLGWYQQKPGKAPKLLIYGASSLQSG | 367 |
| | | VPSRFSGSGSGTEFTLTISSLQPEDFATYYCLQHNNYPFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361071 | 6H1.007 | DIQMTQSPSSLSASIGDRVTITCRASQDIRDYLGWYQQKPGKAPKLLIYGASSLQSG | 368 |
| | | VPSRFSGSGSGTEFTLTISSLQPEDFATYYCLQHNNYPFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361075 | 6H1.008 | DIQMTQSPSSLSASIGDRVTITCRASQDIRDYLGWYQQKPGKAPKLLIYGASSLQSG | 369 |
| | | VPSRFSGSGSGTEFTLTISSLQPEDFATYYCLQHNNYPFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361079 | 6A5.004.001 | DIQMTQSPSSLSASVGDRVTITCQASQDITNYLNWYQQKPGKAPKLLIYDASNLEP | 370 |
| | | GVPSRFSGSGSGTDFTLTISSLQPEDFATYYCQQYDDLFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361085 | 6A5.004.002 | DIQMTQSPSSLSASVGDRVTITCQASQDITNYLNWYQQKPGKAPKLLIYDASNLEP | 371 |
| | | GVPSRFSGSGSGTDFTLTISSLQPEDIATYYCQQYDDLFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361091 | 6A5.004.003 | DIQMTQSPSSLSASVGDRVTITCQASQDITNYLNWYQQKPGKAPKLLIYDASNLEP | 372 |
| | | GVPSRFSGSGSGTDFTLTISSLQPEDFATYYCQQYDDLFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361095 | 6A5.004.004 | DIQMTQSPSSLSASVGDRVTITCQASQDITNYLNWYQQKPGKAPKLLIYDASNLEP | 373 |
| | | GVPSRFSGSGSGTDFTFTISSLQPEDFATYYCQQYDDLFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361101 | 6A5.004.005 | DIQMTQSPSSLSASVGDRVTITCQASQDITNYLNWYQQKPGKAPKLLIYDASNLEP | 374 |
| | | GVPSRFSGSGSGTDFTLTISSLQPEDIATYYCQQYDDLFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361105 | 6A5.004.006 | DIQMTQSPSSLSASVGDRVTITCQASQDITNYLNWYQQKPGKAPKLLIYDASNLEP | 375 |
| | | GVPSRFSGSGSGTDFTFTISSLQPEDFATYYCQQYDDLFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361109 | 6A5.004.007 | DIQMTQSPSSLSASVGDRVTITCQASQDITNYLNWYQQKPGKAPKLLIYDASNLEP | 376 |
| | | GVPSRFSGSGSGTDFTFTISSLQPEDIATYYCQQYDDLFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361113 | 6A5.004.008 | DIQMTQSPSSLSASVGDRVTITCQASQDITNYLNWYQQKPGKAPKLLIYDASNLEP | 377 |
| | | GVPSRFSGSGSGTDFTLTISSLQPEDFATYYCQQYDDLFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361120 | 6A5.004.009 | DIQMTQSPSSLSASVGDRVTITCQASQDITNYLNWYQQKPGKAPKLLIYDASNLEP | 378 |
| | | GVPSRFSGSGSGTDFTLTISSLQPEDFATYYCQQYDDLFTFGPGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:359896 | 6A5.004.010 | DIQMTQSPSSLSASVGDRVTITCQASQDITNYLNWYQQKPGKAPKLLIYDASNLEP | 379 |
| | | GVPSRFSGSGSGTDFTLTISSLQPEDFATYYCQQYDDLFTFGQGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:359890 | 6A5.004.011 | DIQMTQSPSSLSASVGDRVTITCQASQDITNYLNWYQQKPGKAPKLLIYDASNLEP | 380 |
| | | GVPSRFSGSGSGTDFTLTISSLQPEDFATYYCQQYDDLFTFGQGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:359567 | 2A11.002 | EIVLTQSPGTLSLSPGERATLSCRASQIFTSTYLAWYQQKPGQAPRLLIYGASSRAT | 381 |
| | | GIPDRFSGSGSGTDFTLTISRLEPEDFAVYYCQQYGSSPRFGQGTRLEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:335965 | 2A11.003 | EIVLTQSPGTLSLSPGERATLSCRASQIFTSTYLAWYQQKPGQAPRLLIYGASSRAT | 382 |
| | | GIPDRFSGSGSGTDFTLTISRLEPEDFAVYYCQQYGSSPRFGQGTRLEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:361158 | 2A11.004 | EIVLTQSPGTLSLSPGERATLSCRASQIFTSTYLAWYQQKPGQAPRLLIYGASSRAT | 383 |
| | | GIPDRFSGSGSGTDFTLTISRLEPEDFAVYYCQQYGSSPRFGQGTRLEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:361165 | 2A11.005 | EIVLTQSPGTLSLSPGERATLSCRASQIFTSTYLAWYQQKPGQAPRLLIYGASSRAT | 384 |
| | | GIPDRFSGSGSGTDFTLTISRLEPEDFAVYYCQQYGSSPRFGQGTRLEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:361172 | 2G10_LC1.003 | EIVMTQSPATLSVSPGERATLSCRASQSVSSNLAWYQQKPGQAPRLLIYGAATRAT | 385 |
| | | GIPARFSGSGSGTEFTLTISSLQSEDFAVYYCQQYNNWPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361178 | 2G10_LC1.004 | EIVMTQSPATLSVSPGERATLSCRASQSVSSNLAWYQQKPGQAPRLLIYGAATRAT | 386 |
| | | GIPARFSGSGSGTEFTLTISSLQSEDFAVYYCQQYNNWPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361185 | 2G10_LC1.005 | EIVMTQSPATLSVSPGERATLSCRASQSVSSNLAWYQQKPGQAPRLLIYGAATRAT | 387 |
| | | GIPARFSGSGSGTEFTLTISSLQSEDFAVYYCQQYNNWPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361192 | 2G10_LC1.006 | EIVMTQSPATLSVSPGERATLSCRASQSVSSNLAWYQQKPGQAPRLLIYGAATRAT | 388 |
| | | GIPARVSGSGSGTEFTLTISSLQSEDFAVYYCQQYNNWPLTFGGGTKVEIKRTVAA | |
| | | PSVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:359609 | 2G10_LC1.007 | EIVMTQSPATLSVSPGERATLSCRASQSVSSNLAWYQQKPGQAPRLLIYGAATRAT | 389 |
| | | GIPARFSGSGSGTEFTLTISSLEPEDFAVYYCQQYNNFPLTFGGGTKVEIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:359615 | 2G10_LC1.008 | EIVMTQSPATLSVSPGERATLSCRASQSVSSNLAWYQQKPGQAPRLLIYGAATRAT | 390 |
| | | GIPARFSGSGSGTEFTLTISSLEPEDFAVYYCQQYNNYPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361196 | 2G10_LC1.009 | EIVMTQSPATLSVSPGERATLSCRASQSVSSNLAWYQQKPGQAPRLLIYGAATRAT | 391 |
| | | GIPARFSGSGSGTEFTLTISSLEPEDFAVYYCQQYNNWPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361202 | 2G10_LC1.010 | EIVMTQSPATLSVSPGERATLSCRASQSVSSNLAWYQQKPGQAPRLLIYGAATRAT | 392 |
| | | GIPARFSGSGSGTEFTLTISSLEPEDFAVYYCQQYNNWPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:359644 | 18E3.002 | EIVLTQSPGTLSLSPGERATLSCRASQSISYSYLAWYQQKPGQAPRLLIYGASSRAT | 393 |
| | | GIPDRFSGSGSGTDFTLTISRLEPDDFAVYYCQQYGSSPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361206 | 18E3.003 | EIVLTQSPGTLSLSPGERATLSCRASQSISYSYLAWYQQKPGQAPRLLIYGASSRAT | 394 |
| | | GIPDRFSGSGSGTDFTLTISRQEPDDFAVYYCQQYGSSPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:359628 | 18E3.004 | EIVLTQSPGTLSLSPGERATLSCRASQSISYSYLAWYQQKPGQAPRLLIYGASSRAT | 395 |
| | | GIPDRFSGSGSGTDFTLTISRLEPDDFAVYYCQQYGSSPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:359637 | 18E3.005 | EIVLTQSPGTLSLSPGERATLSCRASQSISYSYLAWYQQKPGQAPRLLIYGASSRAT | 396 |
| | | GIPDRFSGSGSGTDFTLTISRLEPDDFAVYYCQQYGSSPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361210 | 18E3.006 | EIVLTQSPGTLSLSPGERATLSCRASQSISYSYLAWYQQKPGQAPRLLIYGASSRAT | 397 |
| | | GIPDRFSGSGSGTDFTLTISRLEPDDFAVYYCQQYGSSPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361214 | 18E3.007 | EIVLTQSPGTLSLSPGERATLSCRASQSISYSYLAWYQQKPGQAPRLLIYGASSRAT | 398 |
| | | GIPDRFSGSGSGTDFTLTISRQEPDDFAVYYCQQYGSSPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361218 | 18E3.008 | EIVLTQSPGTLSLSPGERATLSCRASQSISYSYLAWYQQKPGQAPRLLIYGASSRAT | 399 |
| | | GIPDRFSGSGSGTDFTLTISRQEPDDFAVYYCQQYGSSPLTFGGGTKVEIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361222 | 5C2.006 | DIVMTQSPDSLAVSLGERATINCKSSQSVLSSSNNKNYIAWYQQQPGHPPKLLIYW | 400 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361229 | 5C2.007 | DIVMTQSPDSLAVSLGERATINCKSSQSVLSSSNNKNYIAWYQQQPGHPPKLLIYW | 401 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361236 | 5C2.008 | DIVMTQSPDSLAVSLGERATINCKSSQSVLSSSNNKNYIAWYQQQPGHPPKLLIYW | 402 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:359685 | 5C2.009 | DIVMTQSPDSLAVSLGERATINCKSSQSVLSSSNNKNYIAWYQQQPGHPPKLLIYW | 403 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361240 | 5C2.010 | DIVMTQSPDSLAVSLGERATINCKSSQSVLSSSNNKNYIAWYQQQPGHPPKLLIYW | 404 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361247 | 5C2.011 | DIVMTQSPDSLAVSLGERATINCKSSQSVLSSSNNKNYIAWYQQQPGHPPKLLIYW | 405 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361254 | 5C2.012 | DIVMTQSPDSLAVSLGERATINCKSSQSVLSSSNNKNYIAWYQQQPGHPPKLLIYW | 406 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361261 | 5C2.013 | DIVMTQSPDSLAVSLGERATINCKSSQSVLSSSNNKNYIAWYQQQPGHPPKLLIYW | 407 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361268 | 5C2.014 | DIVMTQSPDSLAVSLGERATINCKSSQSVLSSSNNKNYIAWYQQQPGHPPKLLIYW | 408 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:359669 | 5C2.015 | DIVMTQSPDSLAVSLGERATINCKSSQSVLSSSNNKNYIAWYQQKPGQPPKLLIYW | 409 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:359678 | 5C2.016 | DIVMTQSPDSLAVSLGERATINCKSSQSVLSSSNNKNYIAWYQQKPGQPPKLLIYW | 410 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361275 | 11C1.002 | DIVMTQSPDSLSVSLGERATINCKSSQSVLSSSNNKNYLAWYQQKPGQPPNLLIYW | 411 |
| | | TSTRESGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361282 | 11C1.003 | DIVMTQSPDSLSVSLGERATINCKSSQSVLSSSNNKNYLAWYQQKPGQPPNLLIYW | 412 |
| | | TSTRESGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361289 | 11C1.004 | DIVMTQSPDSLSVSLGERATINCKSSQSVLSSSNNKNYLAWYQQKPGQPPNLLIYW | 413 |
| | | TSTRDSGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:359712 | 11C1.005 | DIVMTQSPDSLSVSLGERATINCKSSQSVLSSSNNKNYLAWYQQKPGQPPNLLIYW | 414 |
| | | TSTRESGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361293 | 11C1.006 | DIVMTQSPDSLSVSLGERATINCKSSQSVLSSSNNKNYLAWYQQKPGQPPNLLIYW | 415 |
| | | TSTRESGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361297 | 11C1.007 | DIVMTQSPDSLSVSLGERATINCKSSQSVLSSSNNKNYLAWYQQKPGQPPNLLIYW | 416 |
| | | TSTRDSGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361301 | 11C1.008 | DIVMTQSPDSLSVSLGERATINCKSSQSVLSSSNNKNYLAWYQQKPGQPPNLLIYW | 417 |
| | | TSTRDSGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:359703 | 11C1.009 | DIVMTQSPDSLAVSLGERATINCKSSQSVLSSSNNKNYLAWYQQKPGQPPKLLIYW | 418 |
| | | TSTRESGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361305 | 11C1.010 | DIVMTQSPDSLSVSLGERATINCKSSQSVLSSSNNKNYLAWYQQKPGQPPNLLIYW | 419 |
| | | TSTRESGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361312 | 13H12.002 | DIVMTQSPDSLAVSLGERATINCKSSQSVLSSSNNKNYIAWYQQKPGHPPKLLIYW | 420 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:359744 | 13H12.003 | DIVMTQSPDSLAVSLGERATINCKSSQSVLSSSNNKNYIAWYQQKPGHPPKLLIYW | 421 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361319 | 13H12.004 | DIVMTQSPDSLAVSLGERATINCKSSQSVLSSSNNKNYIAWYQQKPGHPPKLLIYW | 422 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:359737 | 13H12.005 | DIVMTQSPDSLAVSLGERATINCKSSQSVLSSSNNKNYIAWYQQKPGQPPKLLIYW | 423 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361326 | 13H12.006 | DIVMTQSPDSLAVSLGERATINCKSSQSVLSSSNNKNYIAWYQQKPGQPPKLLIYW | 424 |
| | | ASTRESGVPDRFSGSGSGTDFTLTISSLQAEDVAVFYCQQYYSTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361330 | 12H11.002 | DIVMTQSPDSLSVSLGERATINCKSSQSVLSSSNNKNYLAWYQQKPGQPPNLLIYW | 425 |
| | | TSTRDSGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361337 | 12H11.003 | DIVMTQSPDSLSVSLGERATINCKSSQSVLSSSNNKNYLAWYQQKPGQPPNLLIYW | 426 |
| | | TSTRDSGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361344 | 12H11.004 | DIVMTQSPDSLSVSLGERATINCKSSQSVLSSSNNKNYLAWYQQKPGQPPNLLIYW | 427 |
| | | TSTRDSGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361351 | 12H11.005 | DIVMTQSPDSLSVSLGERATINCKSSQSVLSSSNNKNYLAWYQQKPGQPPNLLIYW | 428 |
| | | TSTRDSGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361358 | 12H11.006 | DIVMTQSPDSLSVSLGERATINCKSSQSVLSSSNNKNYLAWYQQKPGQPPNLLIYW | 429 |
| | | TSTRDSGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361365 | 12H11.007 | DIVMTQSPDSLSVSLGERATINCKSSQSVLSSSNNKNYLAWYQQKPGQPPNLLIYW | 430 |
| | | TSTRDSGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361372 | 12H11.008 | DIVMTQSPDSLSVSLGERATINCKSSQSVLSSSNNKNYLAWYQQKPGQPPNLLIYW | 431 |
| | | TSTRDSGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361379 | 12H11.009 | DIVMTQSPDSLSVSLGERATINCKSSQSVLSSSNNKNYLAWYQQKPGQPPNLLIYW | 432 |
| | | TSTRESGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361383 | 12H11.010 | DIVMTQSPDSLSVSLGERATINCKSSQSVLSSSNNKNYLAWYQQKPGQPPNLLIYW | 433 |
| | | TSTRESGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361387 | 12H11.011 | DIVMTQSPDSLSVSLGERATINCKSSQSVLSSSNNKNYLAWYQQKPGQPPNLLIYW | 434 |
| | | TSTRESGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361391 | 12H11.012 | DIVMTQSPDSLSVSLGERATINCKSSQSVLSSSNNKNYLAWYQQKPGQPPNLLIYW | 435 |
| | | TSTRESGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361395 | 12H11.013 | DIVMTQSPDSLAVSLGERATINCKSSQSVLSSSNNKNYLAWYQQKPGQPPKLLIYW | 436 |
| | | TSTRDSGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361402 | 12H11.014 | DIVMTQSPDSLAVSLGERATINCKSSQSVLSSSNNKNYLAWYQQKPGQPPKLLIYW | 437 |
| | | TSTRESGVPDRFSGSGSGTDFTLTINSLQAEDVAVYYCQQYYRTPWTFGQGTKVEI | |
| | | KRTVAAPSVFIFPPSDEQLKSGTASVVCLLNNFYPREAKVQWKVDNALQSGNSQE | |
| | | SVTEQDSKDSTYSLSSTLTLSKADYEKHKVYACEVTHQGLSSPVTKSFNRGEC | |
| iPS:361406 | 2C2.005.001 | QSVLTQPPSASGTPGQRVTISCSGSSSNIGSQTVNWYQHLPGTAPKLLIYTNNQRPS | 438 |
| | | GVPDRFSGSKSGTSASLAISGLQSEDEADYFCATFDESLSGPVFGGGTKLTVLGQPK | |
| | | AAPSVTLFPPSSEELQANKATLVCLISDFYPGAVTVAWKADSSPVKAGVETTTPSK | |
| | | QSNNKYAASSYLSLTPEQWKSHRSYSCQVTHEGSTVEKTVAPTECS | |
| iPS:361412 | 2C2.005.002 | QSVLTQPPSASGTPGQRVTISCSGSSSNIGSQTVNWYQHLPGTAPKLLIYTNNQRPS | 439 |
| | | GVPDRFSGSKSGTSASLAISGLQSEDEADYFCATFDESLQGPVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361418 | 2C2.005.003 | QSVLTQPPSASGTPGQRVTISCSGSSSNIGSNYVNWYQHLPGTAPKLLIYTNNQRPS | 440 |
| | | GVPDRFSGSKSGTSASLAISGLQSEDEADYFCATFDESLSGPVFGGGTKLTVLGQPK | |
| | | AAPSVTLFPPSSEELQANKATLVCLISDFYPGAVTVAWKADSSPVKAGVETTTPSK | |
| | | QSNNKYAASSYLSLTPEQWKSHRSYSCQVTHEGSTVEKTVAPTECS | |
| iPS:361424 | 2C2.005.004 | QSVLTQPPSASGTPGQRVTISCSGSSSNIGSNYVNWYQHLPGTAPKLLIYTNNQRPS | 441 |
| | | GVPDRFSGSKSGTSASLAISGLQSEDEADYFCATFDESLQGPVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361430 | 2C2.005.005 | QSVLTQPPSASGTPGQRVTISCSGSSSNIGSQTVNWYQHLPGTAPKLLIYTNNQRPS | 442 |
| | | GVPDRFSGSKSGTSASLAISGLQSEDEADYFCATFDDSLNGPVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361436 | 2C2.005.006 | QSVLTQPPSASGTPGQRVTISCSGSSSNIGSNYVNWYQHLPGTAPKLLIYTNNQRPS | 443 |
| | | GVPDRFSGSKSGTSASLAISGLQSEDEADYFCATFDDSLNGPVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361442 | 2C2.005.007 | QSVLTQPPSASGTPGQRVTISCSGSSSNIGSNTVNWYQHLPGTAPKLLIYTNNQRPS | 444 |
| | | GVPDRFSGSKSGTSASLAISGLQSEDEADYFCATFDESLNGPVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361448 | 2C2.005.008 | QSVLTQPPSASGTPGQRVTISCSGSSSNIGSNTVNWYQHLPGTAPKLLIYTNNQRPS | 445 |
| | | GVPDRFSGSKSGTSASLAISGLQSEDEADYFCATFDDSLQGPVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361454 | 2C2.005.009 | QSVLTQPPSASGTPGQRVTISCSGSSSNIGSNTVNWYQHLPGTAPKLLIYTNNQRPS | 446 |
| | | GVPDRFSGSKSGTSASLAISGLQSEDEADYFCATFDDSLSGPVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361460 | 2C2.005.010 | QSVLTQPPSASGTPGQRVTISCSGSSSNIGSNTVNWYQHLPGTAPKLLIYTNNQRPS | 447 |
| | | GVPDRFSGSKSGTSASLAISGLQSEDEADYFCATFDDSLNAPVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361466 | 2C2.005.011 | QSVLTQPPSASGTPGQRVTISCSGSSSNIGSNTVNWYQHLPGTAPKLLIYTNNQRPS | 448 |
| | | GVPDRFSGSKSGTSASLAISGLQSEDEADYFCATFDSSLNGPVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361472 | 2C2.005.012 | QSVLTQPPSASGTPGQRVTISCSGSSSNIGSNYVNWYQHLPGTAPKLLIYTNNQRPS | 449 |
| | | GVPDRFSGSKSGTSASLAISGLQSEDEADYFCATFDSSLNAPVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361478 | 2C2.005.013 | QSVLTQPPSASGTPGQRVTISCSGSSSNIGSNYVNWYQHLPGTAPKLLIYTNNQRPS | 450 |
| | | GVPDRFSGSKSGTSASLAISGLQSEDEADYFCATFDSSLNAPVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361485 | 2C2.005.014 | QSVLTQPPSASGTPGQRVTISCSGSSSNIGSQTVNWYQHLPGTAPKLLIYTNNQRPS | 451 |
| | | GVPDRFSGSKSGTSASLAISGLQSEDEADYFCATFDESLSGPVFGGGTKLTVLGQPK | |
| | | AAPSVTLFPPSSEELQANKATLVCLISDFYPGAVTVAWKADSSPVKAGVETTTPSK | |
| | | QSNNKYAASSYLSLTPEQWKSHRSYSCQVTHEGSTVEKTVAPTECS | |
| iPS:361845 | 18F2.013 | DIQMTQSPSSLSASIGDRVTITCRASQDLRNYLGWYQQKPGKAPKRLIYGASSLQS | 452 |
| | | GVPSRFSGSGSGTEFTLTISSLQPEDFATYYCLQHNNYPFTFGQGTKVDIKRTVAAP | |
| | | SVFIFPPSDEQLKSGTASVVCLLNNFYPREAKVQWKVDNALQSGNSQESVTEQDS | |
| | | KDSTYSLSSTLTLSKADYEKHKVYACEVTHQGLSSPVTKSFNRGEC | |
| iPS:361851 | 6H1.009 | DIQMTQSPSSLSASIGDRVTITCRASQDIRDYLGWYQQKPGKAPKRLIYGASSLQSG | 453 |
| | | VPSRFSGSGSGTEFTLTISSLQPEDFATYYCLQHNNYPFTFGQGTKVDIKRTVAAPS | |
| | | VFIFPPSDEQLKSGTASVVCLLNNFYPREAKVQWKVDNALQSGNSQESVTEQDSK | |
| | | DSTYSLSSTLTLSKADYEKHKVYACEVTHQGLSSPVTKSFNRGEC | |
| iPS:361855 | 2C2.005.015 | QSVLTQPPSASGTPGQRVTISCSGSSSNIGSNTVNWYQQLPGTAPKLLIYTNNQRPS | 454 |
| | | GVPDRFSGSKSGTSASLAISGLQSEDEADYFCATFDSSLSGPVFGGGTKLTVLGQPK | |
| | | AAPSVTLFPPSSEELQANKATLVCLISDFYPGAVTVAWKADSSPVKAGVETTTPSK | |
| | | QSNNKYAASSYLSLTPEQWKSHRSYSCQVTHEGSTVEKTVAPTECS | |
| iPS:336067 | 5G12.006 | DIQLTQSPSSLSASVGDRVTITCRASQTISRFLNWYQQKPGKAPELLIYVASSLQSG | 455 |
| | | VPSRFSGSGSGTDFTLTISSLQPEDFATYYCQQSYSTLISFGQGTKLEITRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:336169 | 17B11.002 | QSVLTQPPSVSAAPGQKVTISCSGSSSNIGNNYVSWYEQFPGTAPKLLIYDNNKRPS | 456 |
| | | GIPDRFSGSKSGTSATLGITGLQTGDEADYYCGTWDSSLSAVVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361127 | 5G12.006.001 | DIQLTQSPSSLSASVGDRVTITCRASQTISRFLNWYQQKPGKAPELLIYVASSLQSG | 457 |
| | | VPSRFSGSGSGTDFTLTISSLQPEDFATYYCQQSYSTLISFGQGTKLEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:361136 | 5G12.006.002 | DIQLTQSPSSLSASVGDRVTITCRASQTISRFLNWYQQKPGKAPELLIYVASSLQSG | 458 |
| | | VPSRFSGSGSGTDFTLTISSLQPEDFATYYCQQSYSTLISFGQGTKLEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:361140 | 5G12.006.003 | DIQLTQSPSSLSASVGDRVTITCRASQTISRFLNWYQQKPGKAPELLIYVASSLQSG | 459 |
| | | VPSRFSGSGSGTDFTLTISSLQPEDFATYYCQQSYSTLISFGQGTKLEITRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:359919 | 5G12.006.004 | DIQLTQSPSSLSASVGDRVTITCRASQTISRFLNWYQQKPGKAPKLLIYVASSLQSG | 460 |
| | | VPSRFSGSGSGTDFTLTISSLQPEDFATYYCQQSYSTLISFGQGTKLEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:361144 | 5G12.006.005 | DIQLTQSPSSLSASVGDRVTITCRASQTISRFLNWYQQKPGKAPELLIYVASSLQSG | 461 |
| | | VPSRFSGSGSGTDFTLTISSLQPEDFATYYCQQSYSTLISFGQGTKLEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:361151 | 5G12.006.006 | DIQLTQSPSSLSASVGDRVTITCRASQTISRFLNWYQQKPGKAPELLIYVASSLQSG | 462 |
| | | VPSRFSGSGSGTDFTLTISSLQPEDFATYYCQQSYSTLISFGQGTKLEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |
| iPS:361491 | 17B11.002.001 | QSVLTQPPSVSAAPGQKVTISCSGSSSNIGNNYVSWYQQLPGTAPKLLIYDNNKRPS | 463 |
| | | GIPDRFSGSKSGTSATLGITGLQTGDEADYYCGTWDSSLSAVVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361499 | 17B11.002.002 | QSVLTQPPSVSAAPGQKVTISCSGSSSNIGNNYVSWYQQLPGTAPKLLIYDNNKRPS | 464 |
| | | GIPDRFSGSKSGTSATLGITGLQTGDEADYYCGTWDSSLSAVVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361503 | 17B11.002.003 | QSVLTQPPSVSAAPGQKVTISCSGSSSNIGNNYVSWYEQFPGTAPKLLIYDNNKRPS | 465 |
| | | GIPDRFSGSKSGTSATLGITGLQTGDEADYYCGTWDSSLSAVVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361507 | 17B11.002.004 | QSVLTQPPSVSAAPGQKVTISCSGSSSNIGNNYVSWYQQLPGTAPKLLIYDNNKRPS | 466 |
| | | GIPDRFSGSKSGTSATLAITGLQTGDEADYYCGTFESSLSAVVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361514 | 17B11.002.005 | QSVLTQPPSVSAAPGQKVTISCSGSSSNIGNNYVSWYQQLPGTAPKLLIYDNNKRPS | 467 |
| | | GIPDRFSGSKSGTSATLAITGLQTGDEADYYCGTWDSSLSAVVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:360582 | 17B11.002.006 | QSVLTQPPSVSAAPGQKVTISCSGSSSNIGNNYVSWYQQLPGTAPKLLIYDNNKRPS | 468 |
| | | GIPDRFSGSKSGTSATLAITGLQTGDEADYYCGTWDSSLSAVVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:361518 | 17B11.002.007 | QSVLTQPPSVSAAPGQKVTISCSGSSSNIGNNYVSWYQQLPGTAPKLLIYDNNKRPS | 469 |
| | | GIPDRFSGSKSGTSATLGITGLQTGDEADYYCGTWDSSLSAVVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:360570 | 17B11.002.008 | QSVLTQPPSVSAAPGQKVTISCSGSSSNIGNNYVSWYQQLPGTAPKLLIYDNNKRPS | 470 |
| | | GIPDRFSGSKSGTSATLAITGLQTGDEADYYCGTFESSLSAVVFGGGTKLTVLGQP | |
| | | KAAPSVTLFPPSSEELQANKATLVCLISDFYPGAVTVAWKADSSPVKAGVETTTPS | |
| | | KQSNNKYAASSYLSLTPEQWKSHRSYSCQVTHEGSTVEKTVAPTECS | |
| iPS:362051 | 5G12.005.001 | DIQLTQSPSSLSASVGDRVTITCRASQTISRFLNWYQQKPGKAPKLLIYVASSLQSG | 471 |
| | | VPSRFSGSGSGTDFTLTISSLQPEDFATYYCQQSYSTLLSFGQGTKLEIKRTVAAPSV | |
| | | FIFPPSDEQLKSGTASVVCLLNNFYPREAKVQWKVDNALQSGNSQESVTEQDSKD | |
| | | STYSLSSTLTLSKADYEKHKVYACEVTHQGLSSPVTKSFNRGEC | |

TABLE 15
Heavy Chain (HC) Amino Acid Sequences of Example Anti-GIPR Antibodies

| | | | SEQ |
|---|---|---|---|
| Identifier | Construct | HC | ID NO: |
| iPS:336175 | 2C2.005 | QMQVVQSGAEVKKPGASVKVSCKASGYTFTGYYMHWVRQAPGQGLEWMGWIN | 472 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:335914 | 4B1 | QVQLVESGGGVVQPGRSLRLSCAASGFTFSNFGMHWVRQAPGKGLEWVAVIWY | 473 |
| | | DGSNENYADSVKGRFTISRDNSKNTLYLHMNSLRVADTAVYYCARDGTIFGVVLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:335938 | 6H1 | QVQLVESGGGVVQPGRSLRLSCAASGFTFSYFGMHWVRQAPGKGLEWVAVIWY | 474 |
| | | DGSNKYYADSVKGRFTISRDNSKNTLYLQMNSLRAEDTAVYYCARDGTIFGVLLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:335941 | 2F11 | QVQLVESGGGVVQPGRSLRLSCAASGFTFSSYGMHWVRQAPGKGLEWVAVIWY | 475 |
| | | DGSNKYYADSVKGRFTISSDNSKNTLYLQMNSLRAEDTAVYYCARDRTIFGVVLD | |
| | | YWGQGTLVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:335970 | 5C2 | QVPLVQSGAEVKKPGASVKVSCKASGYTFTGYYIHWVRQAPGQGLEWMGWINP | 476 |
| | | DSGGTDYSQRFQGRFTMTRDTSISTAYMELNRLRSDDTAVYYCAREATIFGMVIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:335978 | 13H12 | QVPLVQSGAEVKKPGASVKVSCKASGYTFTGYYIHWVRQAPGQGLEWMGWINP | 477 |
| | | DSGGTDYSQRFQGRFTMTRDTSISTAYMELSRLRSDDTAVYYCAREATIFGMVIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:335986 | 11C1 | QVPLVQSGAEVKKPGASVKVSCKASGYTFTGYYIHWVRQAPGQGLVWMGWISP | 478 |
| | | NSGDTSYAQKFQDRVTMTRDTSISTAYMELSRLRSDDTAVYFCTREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:335994 | 12H11 | QVPLVQSGAEVKKPGASVKVSCKASGYTFTGYYIHWVRQAPGQGLEWMGWISPN | 479 |
| | | NGDTNYAQKFQDRVTMTRDTSISTAYMELSRLRSDDTAVYYCTREATIFGMLIVPF | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:336024 | 18E3 | QVQLQESGPGLVKPSQTLSLTCTVSGDSISSGGYYWSWIRQHPGKGLEWIGYIYYS | 480 |
| | | GSTYYNPSLKSRVTISVDTSKNQFSLKLSSVTAADTAVYYCARDRITIFGVVMGGG | |
| | | MDVWGQGTTVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:336041 | 2G10 LC1 | QVQLVESGGGVVQPGRSLRLSCAASGFTFSNYGMHWVRQAPGEGLEWVAAIWFD | 481 |
| | | GSDKYYADSVKGRFTISRDNSKNTLYLQMNSLRAEDTAVYYCARDQAIFGVVPD | |
| | | YWGQGTLVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:336077 | 4H9.004 | QVQLVESGGGVVQPGRSLRLSCAASGFTFSYFGMHWVRQAPGKGLEWVAVIWY | 482 |
| | | DGSNKYYADSVKGRFTISRDNSKNTLYLQMNSLRAEDTAVYYCARDGTIFGVLLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:336088 | 6A5.004 | QVQLVESGGGVVQPGRSLRLSCAASGFSFSSYGMHWVRQPPGKGLEWVAAIWYD | 483 |
| | | GNNKYYADSVKGRFTISRDSSKNTLYLQMNSLRAEDTAVYYCARDRTIFGVVLEY | |
| | | LGQGTLVTVSSASTKGPSVFPLAPSSKSTSGGTAALGCLVKDYFPEPVTVSWNSGA | |
| | | LTSGVHTFPAVLQSSGLYSLSSVVTVPSSSLGTQTYICNVNHKPSNTKVDKKVEPK | |
| | | SCDKTHTCPPCPAPELLGGPSVFLFPPKPKDTLMISRTPEVTCVVVDVSHEDPEVKF | |
| | | NWYVDGVEVHNAKTKPCEEQYGSTYRCVSVLTVLHQDWLNGKEYKCKVSNKAL | |
| | | PAPIEKTISKAKGQPREPQVYTLPPSREEMTKNQVSLTCLVKGFYPSDIAVEWESNG | |
| | | QPENNYKTTPPVLDSDGSFFLYSKLTVDKSRWQQGNVFSCSVMHEALHNHYTQK | |
| | | SLSLSPGK | |
| iPS:336099 | 17H11.004 | QVQLQESGPGLVKPSETLSLTCTVSQGSISSYYWSWIRQPAGKGLEWIGRIYTSGST | 484 |
| | | NYNPSLKSRVTMSIDTSKNQFSLKLNSVTAADTAVYYCARDVAVAGFDYWGQGT | |
| | | LVTVSSASTKGPSVFPLAPSSKSTSGGTAALGCLVKDYFPEPVTVSWNSGALTSGV | |
| | | HTFPAVLQSSGLYSLSSVVTVPSSSLGTQTYICNVNHKPSNTKVDKKVEPKSCDKT | |
| | | HTCPPCPAPELLGGPSVFLFPPKPKDTLMISRTPEVTCVVVDVSHEDPEVKFNWYV | |
| | | DGVEVHNAKTKPCEEQYGSTYRCVSVLTVLHQDWLNGKEYKCKVSNKALPAPIE | |
| | | KTISKAKGQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQKSLSLS | |
| | | PGK | |
| iPS:359621 | 17H11.004.001 | QVQLQESGPGLVKPSETLSLTCTVSGGSISSYYWSWIRQPAGKGLEWIGRIYTSGST | 485 |
| | | NYNPSLKSRVTMSIDTSKNQFSLKLNSVTAADTAVYYCARDVAVAGFDYWGQGT | |
| | | LVTVSSASTKGPSVFPLAPSSKSTSGGTAALGCLVKDYFPEPVTVSWNSGALTSGV | |
| | | HTFPAVLQSSGLYSLSSVVTVPSSSLGTQTYICNVNHKPSNTKVDKKVEPKSCDKT | |
| | | HTCPPCPAPELLGGPSVFLFPPKPKDTLMISRTPEVTCVVVDVSHEDPEVKFNWYV | |
| | | DGVEVHNAKTKPCEEQYGSTYRCVSVLTVLHQDWLNGKEYKCKVSNKALPAPIE | |
| | | KTISKAKGQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQKSLSLS | |
| | | PGK | |
| iPS:359781 | 4H9.004.001 | QVQLVESGGGVVQPGRSLRLSCAASGFTFSYFGMHWVRQAPGKGLEWVAVIWY | 486 |
| | | DASNKYYADAVKGRFTISRDNSKNTLYLQMNSLRAEDTAVYYCARDGTIFGVLL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:360929 | 4H9.004.002 | QVQLVESGGGVVQPGRSLRLSCAASGFTFSYFGMHWVRQAPGKGLEWVAVIWY | 487 |
| | | DASNKYYADSVKGRFTISRDNSKNTLYLQMNSLRAEDTAVYYCARDGTIFGVLLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:360936 | 4H9.004.003 | QVQLVESGGGVVQPGRSLRLSCAASGFTFSYFGMHWVRQAPGKGLEWVAVIWY | 488 |
| | | DGSNKYYADAVKGRFTISRDNSKNTLYLQMNSLRAEDTAVYYCARDGTIFGVLL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:360943 | 4H9.004.004 | QVQLVESGGGVVQPGRSLRLSCAASGFTFSYFGMHWVRQAPGKGLEWVAVIWY | 489 |
| | | DASNKYYADAVKGRFTISRDNSKNTLYLQMNSLRAEDTAVYYCARDGTIFGVLL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:360949 | 4H9.004.005 | QVQLVESGGGVVQPGRSLRLSCAASGFTFSYFGMHWVRQAPGKGLEWVAVIWY | 490 |
| | | DASNKYYADAVKGRFTISRDNSKNTLYLQMNSLRAEDTAVYYCARDGTIFGVLL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:359761 | 4H9.004.006 | QVQLVESGGGVVQPGRSLRLSCAASGFTFSYFGMHWVRQAPGKGLEWVAVIWY | 491 |
| | | DASNKYYADAVKGRFTISRDNSKNTLYLQMNSLRAEDTAVYYCARDRTIFGVLLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:360955 | 4B1.010 | QVQLVESGGGVVQPGRSLRLSCAASGFTFSNFGMHWVRQAPGKGLEWVAVIWY | 492 |
| | | DASNENYADAVKGRFTISRDNSKNTLYLQMNSLRVADTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:360962 | 4B1.011 | QVQLVESGGGVVQPGRSLRLSCAASGFTFSNFGMHWVRQAPGKGLEWVAVIWY | 493 |
| | | DASNENYADAVKGRFTISRDNSKNTLYLQMNSLRVADTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:360968 | 4B1.012 | QVQLVESGGGVVQPGRSLRLSCAASGFTFSNFGMHWVRQAPGKGLEWVAVIWY | 494 |
| | | DASNENYADAVKGRFTISRDNSKNTLYLQMNSLRVADTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:360974 | 4B1.013 | QVQLVESGGGVVQPGRSLRLSCAASGFTFSNFGMHWVRQAPGKGLEWVAVIWY | 495 |
| | | DASNENYADSVKGRFTISRDNSKNTLYLHMNSLRVADTAVYYCARDGTIFGVVLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:360978 | 4B1.014 | QVQLVESGGGVVQPGRSLRLSCAASGFTFSNFGMHWVRQAPGKGLEWVAVIWY | 496 |
| | | DGSNENYADAVKGRFTISRDNSKNTLYLHMNSLRVADTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:359785 | 4B1.015 | QVQLVESGGGVVQPGRSLRLSCAASGFTFSNFGMHWVRQAPGKGLEWVAVIWY | 497 |
| | | DASNENYADAVKGRFTISRDNSKNTLYLQMNSLRVEDTAVYYCARDRTIFGVVLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:360982 | 4B1.016 | QVQLVESGGGVVQPGRSLRLSCAASGFTFSNFGMHWVRQAPGKGLEWVAVIWY | 498 |
| | | DASNENYADAVKGRFTISRDNSKNTLYLQMNSLRVEDTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:335922 | 18F2 | QVQLVESGGGVVQPGRSLRLSCAASGFTFSNFGMHWVRQAPGKGLEWVAVIWY | 499 |
| | | DGSNENYADSVKGRFTISRDNSKNTLYLHMNSLRAADTAVYYCARDGTIFGVVLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:360986 | 18F2.002 | QVQLVESGGGVVQPGRSLRLSCAASGFTFSNFGMHWVRQAPGKGLEWVAVIWY | 500 |
| | | DASNENYADAVKGRFTISRDNSKNTLYLQMNSLRAADTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:360995 | 18F2.003 | QVQLVESGGGVVQPGRSLRLSCAASGFTFSNFGMHWVRQAPGKGLEWVAVIWY | 501 |
| | | DASNENYADAVKGRFTISRDNSKNTLYLHMNSLRAADTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:360999 | 18F2.004 | QVQLVESGGGVVQPGRSLRLSCAASGFTFSNFGMHWVRQAPGKGLEWVAVIWY | 502 |
| | | DASNENYADAVKGRFTISRDNSKNTLYLQMNSLRAADTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361005 | 18F2.005 | QVQLVESGGGVVQPGRSLRLSCAASGFTFSNFGMHWVRQAPGKGLEWVAVIWY | 503 |
| | | DASNENYADAVKGRFTISRDNSKNTLYLQMNSLRAADTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361009 | 18F2.006 | QVQLVESGGGVVQPGRSLRLSCAASGFTFSNFGMHWVRQAPGKGLEWVAVIWY | 504 |
| | | DASNENYADAVKGRFTISRDNSKNTLYLHMNSLRAADTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361013 | 18F2.007 | QVQLVESGGGVVQPGRSLRLSCAASGFTFSNFGMHWVRQAPGKGLEWVAVIWY | 505 |
| | | DASNENYADAVKGRFTISRDNSKNTLYLHMNSLRAADTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361017 | 18F2.008 | QVQLVESGGGVVQPGRSLRLSCAASGFTFSNFGMHWVRQAPGKGLEWVAVIWY | 506 |
| | | DASNENYADAVKGRFTISRDNSKNTLYLQMNSLRAADTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361021 | 18F2.009 | QVQLVESGGGVVQPGRSLRLSCAASGFTFSNFGMHWVRQAPGKGLEWVAVIWY | 507 |
| | | DGSNENYADAVKGRFTISRDNSKNTLYLQMNSLRAADTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361028 | 18F2.010 | QVQLVESGGGVVQPGRSLRLSCAASGFTFSNFGMHWVRQAPGKGLEWVAVIWY | 508 |
| | | DASNENYADSVKGRFTISRDNSKNTLYLQMNSLRAADTAVYYCARDGTIFGVVLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:360535 | 18F2.011 | QVQLVESGGGVVQPGRSLRLSCAASGFTFSNFGMHWVRQAPGKGLEWVAVIWY | 509 |
| | | DASNENYADAVKGRFTISRDNSKNTLYLQMNSLRAEDTAVYYCARDRTIFGVVLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361035 | 18F2.012 | QVQLVESGGGVVQPGRSLRLSCAASGFTFSNFGMHWVRQAPGKGLEWVAVIWY | 510 |
| | | DASNENYADAVKGRFTISRDNSKNTLYLQMNSLRAEDTAVYYCARDGTIFGVVL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:359940 | 2F11.002 | QVQLVESGGGVVQPGRSLRLSCAASGFTFSSYGMHWVRQAPGKGLEWVAVIWY | 511 |
| | | DASNKYYADAVKGRFTISRDNSKNTLYLQMNSLRAEDTAVYYCARDRTIFGVVL | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361039 | 2F11.003 | QVQLVESGGGVVQPGRSLRLSCAASGFTFSSYGMHWVRQAPGKGLEWVAVIWY | 512 |
| | | DASNKYYADAVKGRFTISSDNSKNTLYLQMNSLRAEDTAVYYCARDRTIFGVVLD | |
| | | YWGQGTLVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:361043 | 2F11.004 | QVQLVESGGGVVQPGRSLRLSCAASGFTFSSYGMHWVRQAPGKGLEWVAVIWY | 513 |
| | | DASNKYYADAVKGRFTISRDNSKNTLYLQMNSLRAEDTAVYYCARDRTIFGVVL | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361049 | 2F11.005 | QVQLVESGGGVVQPGRSLRLSCAASGFTFSSYGMHWVRQAPGKGLEWVAVIWY | 514 |
| | | DASNKYYADAVKGRFTISRDNSKNTLYLQMNSLRAEDTAVYYCARDRTIFGVVL | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361055 | 2F11.006 | QVQLVESGGGVVQPGRSLRLSCAASGFTFSSYGMHWVRQAPGKGLEWVAVIWY | 515 |
| | | DASNKYYADAVKGRFTISSDNSKNTLYLQMNSLRAEDTAVYYCARDRTIFGVVLD | |
| | | YWGQGTLVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:361059 | 2F11.007 | QVQLVESGGGVVQPGRSLRLSCAASGFTFSSYGMHWVRQAPGKGLEWVAVIWY | 516 |
| | | DASNKYYADAVKGRFTISSDNSKNTLYLQMNSLRAEDTAVYYCARDRTIFGVVLD | |
| | | YWGQGTLVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:361063 | 2F11.008 | QVQLVESGGGVVQPGRSLRLSCAASGFTFSSYGMHWVRQAPGKGLEWVAVIWY | 517 |
| | | DASNKYYADAVKGRFTISRDNSKNTLYLQMNSLRAEDTAVYYCARDRTIFGVVL | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:359949 | 2F11.009 | QVQLVESGGGVVQPGRSLRLSCAASGFTFSSYGMHWVRQAPGKGLEWVAVIWY | 518 |
| | | DASNKYYADSVKGRFTISRDNSKNTLYLQMNSLRAEDTAVYYCARDRTIFGVVLD | |
| | | YWGQGTLVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:359956 | 2F11.010 | QVQLVESGGGVVQPGRSLRLSCAASGFTFSSYGMHWVRQAPGKGLEWVAVIWY | 519 |
| | | DGSNKYYADAVKGRFTISRDNSKNTLYLQMNSLRAEDTAVYYCARDRTIFGVVL | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:359865 | 6H1.002 | QVQLVESGGGVVQPGRSLRLSCAASGFTFSYFGMHWVRQAPGKGLEWVAVIWY | 520 |
| | | DASNKYYADAVKGRFTISRDNSKNTLYLQMNSLRAEDTAVYYCARDGTIFGVLL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:359869 | 6H1.003 | QVQLVESGGGVVQPGRSLRLSCAASGFTFSYFGMHWVRQAPGKGLEWVAVIWY | 521 |
| | | DASNKYYADAVKGRFTISRDNSKNTLYLQMNSLRAEDTAVYYCARDRTIFGVLLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:359873 | 6H1.004 | QVQLVESGGGVVQPGRSLRLSCAASGFTFSYFGMHWVRQAPGKGLEWVAVIWY | 522 |
| | | DASNKYYADAVKGRFTISRDNSKNTLYLQMNSLRAEDTAVYYCARDGTIFGVLL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:359877 | 6H1.005 | QVQLVESGGGVVQPGRSLRLSCAASGFTFSYFGMHWVRQAPGKGLEWVAVIWY | 523 |
| | | DASNKYYADAVKGRFTISRDNSKNTLYLQMNSLRAEDTAVYYCARDRTIFGVLLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361067 | 6H1.006 | QVQLVESGGGVVQPGRSLRLSCAASGFTFSYFGMHWVRQAPGKGLEWVAVIWY | 524 |
| | | DGSNKYYADSVKGRFTISRDNSKNTLYLQMNSLRAEDTAVYYCARDGTIFGVLLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361071 | 6H1.007 | QVQLVESGGGVVQPGRSLRLSCAASGFTFSYFGMHWVRQAPGKGLEWVAVIWY | 525 |
| | | DASNKYYADSVKGRFTISRDNSKNTLYLQMNSLRAEDTAVYYCARDGTIFGVLLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361075 | 6H1.008 | QVQLVESGGGVVQPGRSLRLSCAASGFTFSYFGMHWVRQAPGKGLEWVAVIWY | 526 |
| | | DGSNKYYADAVKGRFTISRDNSKNTLYLQMNSLRAEDTAVYYCARDGTIFGVLL | |
| | | GDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361079 | 6A5.004.001 | QVQLVESGGGVVQPGRSLRLSCAASGFSFSSYGMHWVRQPPGKGLEWVAAIWYD | 527 |
| | | ANNKYYADAVKGRFTISRDSSKNTLYLQMNSLRAEDTAVYYCARDRTIFGVVLEY | |
| | | WGQGTLVTVSSASTKGPSVFPLAPSSKSTSGGTAALGCLVKDYFPEPVTVSWNSG | |
| | | ALTSGVHTFPAVLQSSGLYSLSSVVTVPSSSLGTQTYICNVNHKPSNTKVDKKVEP | |
| | | KSCDKTHTCPPCPAPELLGGPSVFLFPPKPKDTLMISRTPEVTCVVVDVSHEDPEVK | |
| | | FNWYVDGVEVHNAKTKPCEEQYGSTYRCVSVLTVLHQDWLNGKEYKCKVSNKA | |
| | | LPAPIEKTISKAKGQPREPQVYTLPPSREEMTKNQVSLTCLVKGFYPSDIAVEWESN | |
| | | GQPENNYKTTPPVLDSDGSFFLYSKLTVDKSRWQQGNVFSCSVMHEALHNHYTQ | |
| | | KSLSLSPGK | |
| iPS:361085 | 6A5.004.002 | QVQLVESGGGVVQPGRSLRLSCAASGFSFSSYGMHWVRQPPGKGLEWVAAIWYD | 528 |
| | | ANNKYYADAVKGRFTISRDSSKNTLYLQMNSLRAEDTAVYYCARDRTIFGVVLEY | |
| | | WGQGTLVTVSSASTKGPSVFPLAPSSKSTSGGTAALGCLVKDYFPEPVTVSWNSG | |
| | | ALTSGVHTFPAVLQSSGLYSLSSVVTVPSSSLGTQTYICNVNHKPSNTKVDKKVEP | |
| | | KSCDKTHTCPPCPAPELLGGPSVFLFPPKPKDTLMISRTPEVTCVVVDVSHEDPEVK | |
| | | FNWYVDGVEVHNAKTKPCEEQYGSTYRCVSVLTVLHQDWLNGKEYKCKVSNKA | |
| | | LPAPIEKTISKAKGQPREPQVYTLPPSREEMTKNQVSLTCLVKGFYPSDIAVEWESN | |
| | | GQPENNYKTTPPVLDSDGSFFLYSKLTVDKSRWQQGNVFSCSVMHEALHNHYTQ | |
| | | KSLSLSPGK | |
| iPS:361091 | 6A5.004.003 | QVQLVESGGGVVQPGRSLRLSCAASGFSFSSYGMHWVRQPPGKGLEWVAAIWYD | 529 |
| | | ANNKYYADAVKGRFTISRDSSKNTLYLQMNSLRAEDTAVYYCARDRTIFGVVLEY | |
| | | LGQGTLVTVSSASTKGPSVFPLAPSSKSTSGGTAALGCLVKDYFPEPVTVSWNSGA | |
| | | LTSGVHTFPAVLQSSGLYSLSSVVTVPSSSLGTQTYICNVNHKPSNTKVDKKVEPK | |
| | | SCDKTHTCPPCPAPELLGGPSVFLFPPKPKDTLMISRTPEVTCVVVDVSHEDPEVKF | |
| | | NWYVDGVEVHNAKTKPCEEQYGSTYRCVSVLTVLHQDWLNGKEYKCKVSNKAL | |
| | | PAPIEKTISKAKGQPREPQVYTLPPSREEMTKNQVSLTCLVKGFYPSDIAVEWESNG | |
| | | QPENNYKTTPPVLDSDGSFFLYSKLTVDKSRWQQGNVFSCSVMHEALHNHYTQK | |
| | | SLSLSPGK | |
| iPS:361095 | 6A5.004.004 | QVQLVESGGGVVQPGRSLRLSCAASGFSFSSYGMHWVRQPPGKGLEWVAAIWYD | 530 |
| | | ANNKYYADAVKGRFTISRDSSKNTLYLQMNSLRAEDTAVYYCARDRTIFGVVLEY | |
| | | WGQGTLVTVSSASTKGPSVFPLAPSSKSTSGGTAALGCLVKDYFPEPVTVSWNSG | |
| | | ALTSGVHTFPAVLQSSGLYSLSSVVTVPSSSLGTQTYICNVNHKPSNTKVDKKVEP | |
| | | KSCDKTHTCPPCPAPELLGGPSVFLFPPKPKDTLMISRTPEVTCVVVDVSHEDPEVK | |
| | | FNWYVDGVEVHNAKTKPCEEQYGSTYRCVSVLTVLHQDWLNGKEYKCKVSNKA | |
| | | LPAPIEKTISKAKGQPREPQVYTLPPSREEMTKNQVSLTCLVKGFYPSDIAVEWESN | |
| | | GQPENNYKTTPPVLDSDGSFFLYSKLTVDKSRWQQGNVFSCSVMHEALHNHYTQ | |
| | | KSLSLSPGK | |
| iPS:361101 | 6A5.004.005 | QVQLVESGGGVVQPGRSLRLSCAASGFSFSSYGMHWVRQPPGKGLEWVAAIWYD | 531 |
| | | ANNKYYADAVKGRFTISRDSSKNTLYLQMNSLRAEDTAVYYCARDRTIFGVVLEY | |
| | | LGQGTLVTVSSASTKGPSVFPLAPSSKSTSGGTAALGCLVKDYFPEPVTVSWNSGA | |
| | | LTSGVHTFPAVLQSSGLYSLSSVVTVPSSSLGTQTYICNVNHKPSNTKVDKKVEPK | |
| | | SCDKTHTCPPCPAPELLGGPSVFLFPPKPKDTLMISRTPEVTCVVVDVSHEDPEVKF | |
| | | NWYVDGVEVHNAKTKPCEEQYGSTYRCVSVLTVLHQDWLNGKEYKCKVSNKAL | |
| | | PAPIEKTISKAKGQPREPQVYTLPPSREEMTKNQVSLTCLVKGFYPSDIAVEWESNG | |
| | | QPENNYKTTPPVLDSDGSFFLYSKLTVDKSRWQQGNVFSCSVMHEALHNHYTQK | |
| | | SLSLSPGK | |
| iPS:361105 | 6A5.004.006 | QVQLVESGGGVVQPGRSLRLSCAASGFSFSSYGMHWVRQPPGKGLEWVAAIWYD | 532 |
| | | ANNKYYADAVKGRFTISRDSSKNTLYLQMNSLRAEDTAVYYCARDRTIFGVVLEY | |
| | | LGQGTLVTVSSASTKGPSVFPLAPSSKSTSGGTAALGCLVKDYFPEPVTVSWNSGA | |
| | | LTSGVHTFPAVLQSSGLYSLSSVVTVPSSSLGTQTYICNVNHKPSNTKVDKKVEPK | |
| | | SCDKTHTCPPCPAPELLGGPSVFLFPPKPKDTLMISRTPEVTCVVVDVSHEDPEVKF | |
| | | NWYVDGVEVHNAKTKPCEEQYGSTYRCVSVLTVLHQDWLNGKEYKCKVSNKAL | |
| | | PAPIEKTISKAKGQPREPQVYTLPPSREEMTKNQVSLTCLVKGFYPSDIAVEWESNG | |
| | | QPENNYKTTPPVLDSDGSFFLYSKLTVDKSRWQQGNVFSCSVMHEALHNHYTQK | |
| | | SLSLSPGK | |
| iPS:361109 | 6A5.004.007 | QVQLVESGGGVVQPGRSLRLSCAASGFSFSSYGMHWVRQPPGKGLEWVAAIWYD | 533 |
| | | ANNKYYADAVKGRFTISRDSSKNTLYLQMNSLRAEDTAVYYCARDRTIFGVVLEY | |
| | | WGQGTLVTVSSASTKGPSVFPLAPSSKSTSGGTAALGCLVKDYFPEPVTVSWNSG | |
| | | ALTSGVHTFPAVLQSSGLYSLSSVVTVPSSSLGTQTYICNVNHKPSNTKVDKKVEP | |
| | | KSCDKTHTCPPCPAPELLGGPSVFLFPPKPKDTLMISRTPEVTCVVVDVSHEDPEVK | |
| | | FNWYVDGVEVHNAKTKPCEEQYGSTYRCVSVLTVLHQDWLNGKEYKCKVSNKA | |
| | | LPAPIEKTISKAKGQPREPQVYTLPPSREEMTKNQVSLTCLVKGFYPSDIAVEWESN | |
| | | GQPENNYKTTPPVLDSDGSFFLYSKLTVDKSRWQQGNVFSCSVMHEALHNHYTQ | |
| | | KSLSLSPGK | |
| iPS:361113 | 6A5.004.008 | QVQLVESGGGVVQPGRSLRLSCAASGFSFSSYGMHWVRQPPGKGLEWVAAIWYD | 534 |
| | | GNNKYYADAVKGRFTISRDSSKNTLYLQMNSLRAEDTAVYYCARDRTIFGVVLEY | |
| | | WGQGTLVTVSSASTKGPSVFPLAPSSKSTSGGTAALGCLVKDYFPEPVTVSWNSG | |
| | | ALTSGVHTFPAVLQSSGLYSLSSVVTVPSSSLGTQTYICNVNHKPSNTKVDKKVEP | |
| | | KSCDKTHTCPPCPAPELLGGPSVFLFPPKPKDTLMISRTPEVTCVVVDVSHEDPEVK | |
| | | FNWYVDGVEVHNAKTKPCEEQYGSTYRCVSVLTVLHQDWLNGKEYKCKVSNKA | |
| | | LPAPIEKTISKAKGQPREPQVYTLPPSREEMTKNQVSLTCLVKGFYPSDIAVEWESN | |
| | | GQPENNYKTTPPVLDSDGSFFLYSKLTVDKSRWQQGNVFSCSVMHEALHNHYTQ | |
| | | KSLSLSPGK | |
| iPS:361120 | 6A5.004.009 | QVQLVESGGGVVQPGRSLRLSCAASGFSFSSYGMHWVRQPPGKGLEWVAAIWYD | 535 |
| | | ANNKYYADSVKGRFTISRDSSKNTLYLQMNSLRAEDTAVYYCARDRTIFGVVLEY | |
| | | WGQGTLVTVSSASTKGPSVFPLAPSSKSTSGGTAALGCLVKDYFPEPVTVSWNSG | |
| | | ALTSGVHTFPAVLQSSGLYSLSSVVTVPSSSLGTQTYICNVNHKPSNTKVDKKVEP | |
| | | KSCDKTHTCPPCPAPELLGGPSVFLFPPKPKDTLMISRTPEVTCVVVDVSHEDPEVK | |
| | | FNWYVDGVEVHNAKTKPCEEQYGSTYRCVSVLTVLHQDWLNGKEYKCKVSNKA | |
| | | LPAPIEKTISKAKGQPREPQVYTLPPSREEMTKNQVSLTCLVKGFYPSDIAVEWESN | |
| | | GQPENNYKTTPPVLDSDGSFFLYSKLTVDKSRWQQGNVFSCSVMHEALHNHYTQ | |
| | | KSLSLSPGK | |
| iPS:359896 | 6A5.004.010 | QVQLVESGGGVVQPGRSLRLSCAASGFSFSSYGMHWVRQPPGKGLEWVAAIWYD | 536 |
| | | ANNKYYADAVKGRFTISRDSSKNTLYLQMNSLRAEDTAVYYCARDRTIFGVVLEY | |
| | | WGQGTLVTVSSASTKGPSVFPLAPSSKSTSGGTAALGCLVKDYFPEPVTVSWNSG | |
| | | ALTSGVHTFPAVLQSSGLYSLSSVVTVPSSSLGTQTYICNVNHKPSNTKVDKKVEP | |
| | | KSCDKTHTCPPCPAPELLGGPSVFLFPPKPKDTLMISRTPEVTCVVVDVSHEDPEVK | |
| | | FNWYVDGVEVHNAKTKPCEEQYGSTYRCVSVLTVLHQDWLNGKEYKCKVSNKA | |
| | | LPAPIEKTISKAKGQPREPQVYTLPPSREEMTKNQVSLTCLVKGFYPSDIAVEWESN | |
| | | GQPENNYKTTPPVLDSDGSFFLYSKLTVDKSRWQQGNVFSCSVMHEALHNHYTQ | |
| | | KSLSLSPGK | |
| iPS:359890 | 6A5.004.011 | QVQLVESGGGVVQPGRSLRLSCAASGFSFSSYGMHWVRQPPGKGLEWVAAIWYD | 537 |
| | | ANNKYYADAVKGRFTISRDSSKNTLYLQMNSLRAEDTAVYYCARDRTIFGVVLEY | |
| | | LGQGTLVTVSSASTKGPSVFPLAPSSKSTSGGTAALGCLVKDYFPEPVTVSWNSGA | |
| | | LTSGVHTFPAVLQSSGLYSLSSVVTVPSSSLGTQTYICNVNHKPSNTKVDKKVEPK | |
| | | SCDKTHTCPPCPAPELLGGPSVFLFPPKPKDTLMISRTPEVTCVVVDVSHEDPEVKF | |
| | | NWYVDGVEVHNAKTKPCEEQYGSTYRCVSVLTVLHQDWLNGKEYKCKVSNKAL | |
| | | PAPIEKTISKAKGQPREPQVYTLPPSREEMTKNQVSLTCLVKGFYPSDIAVEWESNG | |
| | | QPENNYKTTPPVLDSDGSFFLYSKLTVDKSRWQQGNVFSCSVMHEALHNHYTQK | |
| | | SLSLSPGK | |
| iPS:359567 | 2A11.002 | QVQLVESGGGVVQPGRSLRLSCAASGFSFSSYGMHWVRQAPGKGLEWVAVIWY | 538 |
| | | DASNKYYEDAVKGRFTISRDNSKNTLFLQVNSLRAEDTAVYYCARGITIFGHGFEY | |
| | | WGQGTLVTVSSASTKGPSVFPLAPSSKSTSGGTAALGCLVKDYFPEPVTVSWNSG | |
| | | ALTSGVHTFPAVLQSSGLYSLSSVVTVPSSSLGTQTYICNVNHKPSNTKVDKKVEP | |
| | | KSCDKTHTCPPCPAPELLGGPSVFLFPPKPKDTLMISRTPEVTCVVVDVSHEDPEVK | |
| | | FNWYVDGVEVHNAKTKPCEEQYGSTYRCVSVLTVLHQDWLNGKEYKCKVSNKA | |
| | | LPAPIEKTISKAKGQPREPQVYTLPPSREEMTKNQVSLTCLVKGFYPSDIAVEWESN | |
| | | GQPENNYKTTPPVLDSDGSFFLYSKLTVDKSRWQQGNVFSCSVMHEALHNHYTQ | |
| | | KSLSLSPGK | |
| iPS:335965 | 2A11.003 | QVQLVESGGGVVQPGRSLRLSCAASGFSFSSYGMHWVRQAPGKGLEWVAVIWY | 539 |
| | | DGSNKYYEDSVKGRFTISRDNSKNTLFLQVNSLRAEDTAVYYCARGITIFGHGFEY | |
| | | WGQGTLVTVSSASTKGPSVFPLAPSSKSTSGGTAALGCLVKDYFPEPVTVSWNSG | |
| | | ALTSGVHTFPAVLQSSGLYSLSSVVTVPSSSLGTQTYICNVNHKPSNTKVDKKVEP | |
| | | KSCDKTHTCPPCPAPELLGGPSVFLFPPKPKDTLMISRTPEVTCVVVDVSHEDPEVK | |
| | | FNWYVDGVEVHNAKTKPCEEQYGSTYRCVSVLTVLHQDWLNGKEYKCKVSNKA | |
| | | LPAPIEKTISKAKGQPREPQVYTLPPSREEMTKNQVSLTCLVKGFYPSDIAVEWESN | |
| | | GQPENNYKTTPPVLDSDGSFFLYSKLTVDKSRWQQGNVFSCSVMHEALHNHYTQ | |
| | | KSLSLSPGK | |
| iPS:361158 | 2A11.004 | QVQLVESGGGVVQPGRSLRLSCAASGFSFSSYGMHWVRQAPGKGLEWVAVIWY | 540 |
| | | DGSNKYYEDAVKGRFTISRDNSKNTLFLQVNSLRAEDTAVYYCARGITIFGHGFEY | |
| | | WGQGTLVTVSSASTKGPSVFPLAPSSKSTSGGTAALGCLVKDYFPEPVTVSWNSG | |
| | | ALTSGVHTFPAVLQSSGLYSLSSVVTVPSSSLGTQTYICNVNHKPSNTKVDKKVEP | |
| | | KSCDKTHTCPPCPAPELLGGPSVFLFPPKPKDTLMISRTPEVTCVVVDVSHEDPEVK | |
| | | FNWYVDGVEVHNAKTKPCEEQYGSTYRCVSVLTVLHQDWLNGKEYKCKVSNKA | |
| | | LPAPIEKTISKAKGQPREPQVYTLPPSREEMTKNQVSLTCLVKGFYPSDIAVEWESN | |
| | | GQPENNYKTTPPVLDSDGSFFLYSKLTVDKSRWQQGNVFSCSVMHEALHNHYTQ | |
| | | KSLSLSPGK | |
| iPS:361165 | 2A11.005 | QVQLVESGGGVVQPGRSLRLSCAASGFSFSSYGMHWVRQAPGKGLEWVAVIWY | 541 |
| | | DASNKYYEDSVKGRFTISRDNSKNTLFLQVNSLRAEDTAVYYCARGITIFGHGFEY | |
| | | WGQGTLVTVSSASTKGPSVFPLAPSSKSTSGGTAALGCLVKDYFPEPVTVSWNSG | |
| | | ALTSGVHTFPAVLQSSGLYSLSSVVTVPSSSLGTQTYICNVNHKPSNTKVDKKVEP | |
| | | KSCDKTHTCPPCPAPELLGGPSVFLFPPKPKDTLMISRTPEVTCVVVDVSHEDPEVK | |
| | | FNWYVDGVEVHNAKTKPCEEQYGSTYRCVSVLTVLHQDWLNGKEYKCKVSNKA | |
| | | LPAPIEKTISKAKGQPREPQVYTLPPSREEMTKNQVSLTCLVKGFYPSDIAVEWESN | |
| | | GQPENNYKTTPPVLDSDGSFFLYSKLTVDKSRWQQGNVFSCSVMHEALHNHYTQ | |
| | | KSLSLSPGK | |
| iPS:361172 | 2G10 LC1.003 | QVQLVESGGGVVQPGRSLRLSCAASGFTFSNYGMHWVRQAPGEGLEWVAAIWFD | 542 |
| | | ASDKYYADAVKGRFTISRDNSKNTLYLQMNSLRAEDTAVYYCARDQAIFGVVPD | |
| | | YWGQGTLVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:361178 | 2G10 LC1.004 | QVQLVESGGGVVQPGRSLRLSCAASGFTFSNYGMHWVRQAPGEGLEWVAAIWFD | 543 |
| | | ASDKYYADSVKGRFTISRDNSKNTLYLQMNSLRAEDTAVYYCARDQAIFGVVPD | |
| | | YWGQGTLVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:361185 | 2G10 LC1.005 | QVQLVESGGGVVQPGRSLRLSCAASGFTFSNYGMHWVRQAPGEGLEWVAAIWFD | 544 |
| | | GSDKYYADAVKGRFTISRDNSKNTLYLQMNSLRAEDTAVYYCARDQAIFGVVPD | |
| | | YWGQGTLVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:361192 | 2G10_LC1.006 | QVQLVESGGGVVQPGRSLRLSCAASGFTFSNYGMHWVRQAPGEGLEWVAAIWFD | 545 |
| | | ASDKYYADAVKGRFTISRDNSKNTLYLQMNSLRAEDTAVYYCARDQAIFGVVPD | |
| | | YWGQGTLVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:359609 | 2G10_LC1.007 | QVQLVESGGGVVQPGRSLRLSCAASGFTFSNYGMHWVRQAPGKGLEWVAAIWF | 546 |
| | | DASDKYYADAVKGRFTISRDNSKNTLYLQMNSLRAEDTAVYYCARDQAIFGVVP | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:359615 | 2G10_LC1.008 | QVQLVESGGGVVQPGRSLRLSCAASGFTFSNYGMHWVRQAPGKGLEWVAAIWF | 547 |
| | | DASDKYYADAVKGRFTISRDNSKNTLYLQMNSLRAEDTAVYYCARDQAIFGVVP | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361196 | 2G10 LC1.009 | QVQLVESGGGVVQPGRSLRLSCAASGFTFSNYGMHWVRQAPGEGLEWVAAIWFD | 548 |
| | | ASDKYYADAVKGRFTISRDNSKNTLYLQMNSLRAEDTAVYYCARDQAIFGVVPD | |
| | | YWGQGTLVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:361202 | 2G10_LC1.010 | QVQLVESGGGVVQPGRSLRLSCAASGFTFSNYGMHWVRQAPGKGLEWVAAIWF | 549 |
| | | DASDKYYADAVKGRFTISRDNSKNTLYLQMNSLRAEDTAVYYCARDQAIFGVVP | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:359644 | 18E3.002 | QVQLQESGPGLVKPSETLSLTCTVSGDSISSGGYYWSWIRQPPGKGLEWIGYIYYS | 550 |
| | | GSTYYNPSLKSRVTISVDTSKNQFSLKLSSVTAADTAVYYCARDRITIFGVVMGGG | |
| | | MDVWGQGTTVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361206 | 18E3.003 | QVQLQESGPGLVKPSETLSLTCTVSGDSISSGGYYWSWIRQPPGKGLEWIGYIYYS | 551 |
| | | GSTYYNPSLKSRVTISVDTSKNQFSLKLSSVTAADTAVYYCARDRITIFGVVMGGG | |
| | | MDVWGQGTTVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:359628 | 18E3.004 | QVQLQESGPGLVKPSQTLSLTCTVSGDSISSGGYYWSWIRQPPGKGLEWIGYIYYS | 552 |
| | | GSTYYNPSLKSRVTISVDTSKNQFSLKLSSVTAADTAVYYCARDRITIFGVVMGGG | |
| | | MDVWGQGTTVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:359637 | 18E3.005 | QVQLQESGPGLVKPSETLSLTCTVSGDSISSGGYYWSWIRQHPGKGLEWIGYIYYS | 553 |
| | | GSTYYNPSLKSRVTISVDTSKNQFSLKLSSVTAADTAVYYCARDRITIFGVVMGGG | |
| | | MDVWGQGTTVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361210 | 18E3.006 | QVQLQESGPGLVKPSQTLSLTCTVSGDSISSGGYYWSWIRQHPGKGLEWIGYIYYS | 554 |
| | | GSTYYNPSLKSRVTISVDTSKNQFSLKLSSVTAADTAVYYCARDRITIFGVVMGGG | |
| | | MDVWGQGTTVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361214 | 18E3.007 | QVQLQESGPGLVKPSETLSLTCTVSGDSISSGGYYWSWIRQHPGKGLEWIGYIYYS | 555 |
| | | GSTYYNPSLKSRVTISVDTSKNQFSLKLSSVTAADTAVYYCARDRITIFGVVMGGG | |
| | | MDVWGQGTTVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361218 | 18E3.008 | QVQLQESGPGLVKPSQTLSLTCTVSGDSISSGGYYWSWIRQPPGKGLEWIGYIYYS | 556 |
| | | GSTYYNPSLKSRVTISVDTSKNQFSLKLSSVTAADTAVYYCARDRITIFGVVMGGG | |
| | | MDVWGQGTTVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361222 | 5C2.006 | QVQLVQSGAEVKKPGASVKVSCKASGYTFTGYYIHWVRQAPGQGLEWMGWINP | 557 |
| | | ESGGTDYSQRFQGRVTMTRDTSISTAYMELNRLRSDDTAVYYCAREATIFGMVIV | |
| | | PFDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361229 | 5C2.007 | QVPLVQSGAEVKKPGASVKVSCKASGYTFTGYYIHWVRQAPGQGLEWMGWINPE | 558 |
| | | SGGTDYSQRFQGRFTMTRDTSISTAYMELNRLRSDDTAVYYCAREATIFGMVIVPF | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361236 | 5C2.008 | QVQLVQSGAEVKKPGASVKVSCKASGYTFTGYYIHWVRQAPGQGLEWMGWINP | 559 |
| | | DAGGTDYSQRFQGRVTMTRDTSISTAYMELNRLRSDDTAVYYCAREATIFGMVIV | |
| | | PFDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:359685 | 5C2.009 | QVPLVQSGAEVKKPGASVKVSCKASGYTFTGYYIHWVRQAPGQGLEWMGWINP | 560 |
| | | DAGGTDYSQRFQGRFTMTRDTSISTAYMELNRLRSDDTAVYYCAREATIFGMVIV | |
| | | PFDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361240 | 5C2.010 | QVQLVQSGAEVKKPGASVKVSCKASGYTFTGYYIHWVRQAPGQGLEWMGWINP | 561 |
| | | DSGGTDYSQRFQGRVTMTRDTSISTAYMELNRLRSDDTAVYYCAREATIFGMVIV | |
| | | PFDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361247 | 5C2.011 | QVQLVQSGAEVKKPGASVKVSCKASGYTFTGYYIHWVRQAPGQGLEWMGWINP | 562 |
| | | DSGGTDYSQRFQGRFTMTRDTSISTAYMELNRLRSDDTAVYYCAREATIFGMVIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361254 | 5C2.012 | QVPLVQSGAEVKKPGASVKVSCKASGYTFTGYYIHWVRQAPGQGLEWMGWINP | 563 |
| | | DSGGTDYSQRFQGRVTMTRDTSISTAYMELNRLRSDDTAVYYCAREATIFGMVIV | |
| | | PFDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361261 | 5C2.013 | QVQLVQSGAEVKKPGASVKVSCKASGYTFTGYYIHWVRQAPGQGLEWMGWINP | 564 |
| | | DAGGTDYSQRFQGRFTMTRDTSISTAYMELNRLRSDDTAVYYCAREATIFGMVIV | |
| | | PFDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361268 | 5C2.014 | QVPLVQSGAEVKKPGASVKVSCKASGYTFTGYYIHWVRQAPGQGLEWMGWINP | 565 |
| | | DAGGTDYSQRFQGRVTMTRDTSISTAYMELNRLRSDDTAVYYCAREATIFGMVIV | |
| | | PFDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:359669 | 5C2.015 | QVQLVQSGAEVKKPGASVKVSCKASGYTFTGYYIHWVRQAPGQGLEWMGWINP | 566 |
| | | DAGGTDYSQRFQGRVTMTRDTSISTAYMELNSLRSDDTAVYYCAREATIFGMVIV | |
| | | PFDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:359678 | 5C2.016 | QVQLVQSGAEVKKPGASVKVSCKASGYTFTGYYIHWVRQAPGQGLEWMGWINP | 567 |
| | | ESGGTDYSQRFQGRVTMTRDTSISTAYMELNSLRSDDTAVYYCAREATIFGMVIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361275 | 11C1.002 | QVPLVQSGAEVKKPGASVKVSCKASGYTFTGYYIHWVRQAPGQGLVWMGWISP | 568 |
| | | NSGETSYAQKFQDRVTMTRDTSISTAYMELSRLRSDDTAVYFCAREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361282 | 11C1.003 | QVPLVQSGAEVKKPGASVKVSCKASGYTFTGYYIHWVRQAPGQGLVWMGWISP | 569 |
| | | NSGDTSYAQKFQDRVTMTRDTSISTAYMELSRLRSDDTAVYFCAREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361289 | 11C1.004 | QVPLVQSGAEVKKPGASVKVSCKASGYTFTGYYIHWVRQAPGQGLVWMGWISP | 570 |
| | | NSGETSYAQKFQDRVTMTRDTSISTAYMELSRLRSDDTAVYFCAREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:359712 | 11C1.005 | QVPLVQSGAEVKKPGASVKVSCKASGYTFTGYYIHWVRQAPGQGLVWMGWISP | 571 |
| | | NSGETSYAQKFQDRVTMTRDTSISTAYMELSRLRSDDTAVYFCTREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361293 | 11C1.006 | QVPLVQSGAEVKKPGASVKVSCKASGYTFTGYYIHWVRQAPGQGLVWMGWISP | 572 |
| | | NSGDTSYAQKFQDRVTMTRDTSISTAYMELSRLRSDDTAVYFCTREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361297 | 11C1.007 | QVPLVQSGAEVKKPGASVKVSCKASGYTFTGYYIHWVRQAPGQGLVWMGWISP | 573 |
| | | NSGETSYAQKFQDRVTMTRDTSISTAYMELSRLRSDDTAVYFCTREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361301 | 11C1.008 | QVPLVQSGAEVKKPGASVKVSCKASGYTFTGYYIHWVRQAPGQGLVWMGWISP | 574 |
| | | NSGDTSYAQKFQDRVTMTRDTSISTAYMELSRLRSDDTAVYFCAREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:359703 | 11C1.009 | QVQLVQSGAEVKKPGASVKVSCKASGYTFTGYYIHWVRQAPGQGLEWMGWISP | 575 |
| | | NSGETSYAQKFQDRVTMTRDTSISTAYMELSSLRSDDTAVYFCAREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361305 | 11C1.010 | QVQLVQSGAEVKKPGASVKVSCKASGYTFTGYYIHWVRQAPGQGLEWMGWISP | 576 |
| | | NSGETSYAQKFQDRVTMTRDTSISTAYMELSRLRSDDTAVYFCAREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361312 | 13H12.002 | QVPLVQSGAEVKKPGASVKVSCKASGYTFTGYYIHWVRQAPGQGLEWMGWINPE | 577 |
| | | SGGTDYSQRFQGRVTMTRDTSISTAYMELSRLRSDDTAVYYCAREATIFGMVIVPF | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:359744 | 13H12.003 | QVPLVQSGAEVKKPGASVKVSCKASGYTFTGYYIHWVRQAPGQGLEWMGWINPE | 578 |
| | | SGGTDYSQRFQGRFTMTRDTSISTAYMELSRLRSDDTAVYYCAREATIFGMVIVPF | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361319 | 13H12.004 | QVPLVQSGAEVKKPGASVKVSCKASGYTFTGYYIHWVRQAPGQGLEWMGWINP | 579 |
| | | DSGGTDYSQRFQGRVTMTRDTSISTAYMELSRLRSDDTAVYYCAREATIFGMVIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:359737 | 13H12.005 | QVQLVQSGAEVKKPGASVKVSCKASGYTFTGYYIHWVRQAPGQGLEWMGWINP | 580 |
| | | ESGGTDYSQRFQGRVTMTRDTSISTAYMELSSLRSDDTAVYYCAREATIFGMVIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361326 | 13H12.006 | QVPLVQSGAEVKKPGASVKVSCKASGYTFTGYYIHWVRQAPGQGLEWMGWINPE | 581 |
| | | SGGTDYSQRFQGRVTMTRDTSISTAYMELSRLRSDDTAVYYCAREATIFGMVIVPF | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361330 | 12H11.002 | QVPLVQSGAEVKKPGASVKVSCKASGYTFTGYYIHWVRQAPGQGLEWMGWISPN | 582 |
| | | QGETNYAQKFQDRVTMTRDTSISTAYMELSRLRSDDTAVYYCAREATIFGMLIVPF | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361337 | 12H11.003 | QVPLVQSGAEVKKPGASVKVSCKASGYTFTGYYIHWVRQAPGQGLEWMGWISPN | 583 |
| | | QGDTNYAQKFQDRVTMTRDTSISTAYMELSRLRSDDTAVYYCAREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361344 | 12H11.004 | QVPLVQSGAEVKKPGASVKVSCKASGYTFTGYYIHWVRQAPGQGLEWMGWISPN | 584 |
| | | QGETNYAQKFQDRVTMTRDTSISTAYMELSRLRSDDTAVYYCTREATIFGMLIVPF | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361351 | 12H11.005 | QVPLVQSGAEVKKPGASVKVSCKASGYTFTGYYIHWVRQAPGQGLEWMGWISPN | 585 |
| | | NGETNYAQKFQDRVTMTRDTSISTAYMELSRLRSDDTAVYYCAREATIFGMLIVPF | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361358 | 12H11.006 | QVPLVQSGAEVKKPGASVKVSCKASGYTFTGYYIHWVRQAPGQGLEWMGWISPN | 586 |
| | | NGDTNYAQKFQDRVTMTRDTSISTAYMELSRLRSDDTAVYYCAREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361365 | 12H11.007 | QVPLVQSGAEVKKPGASVKVSCKASGYTFTGYYIHWVRQAPGQGLEWMGWISPN | 587 |
| | | QGDTNYAQKFQDRVTMTRDTSISTAYMELSRLRSDDTAVYYCTREATIFGMLIVPF | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361372 | 12H11.008 | QVPLVQSGAEVKKPGASVKVSCKASGYTFTGYYIHWVRQAPGQGLEWMGWISPN | 588 |
| | | NGETNYAQKFQDRVTMTRDTSISTAYMELSRLRSDDTAVYYCTREATIFGMLIVPF | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361379 | 12H11.009 | QVPLVQSGAEVKKPGASVKVSCKASGYTFTGYYIHWVRQAPGQGLEWMGWISPN | 589 |
| | | QGETNYAQKFQDRVTMTRDTSISTAYMELSRLRSDDTAVYYCAREATIFGMLIVPF | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361383 | 12H11.010 | QVPLVQSGAEVKKPGASVKVSCKASGYTFTGYYIHWVRQAPGQGLEWMGWISPN | 590 |
| | | NGDTNYAQKFQDRVTMTRDTSISTAYMELSRLRSDDTAVYYCAREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361387 | 12H11.011 | QVPLVQSGAEVKKPGASVKVSCKASGYTFTGYYIHWVRQAPGQGLEWMGWISPN | 591 |
| | | QGDTNYAQKFQDRVTMTRDTSISTAYMELSRLRSDDTAVYYCAREATIFGMLIVP | |
| | | FDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361391 | 12H11.012 | QVPLVQSGAEVKKPGASVKVSCKASGYTFTGYYIHWVRQAPGQGLEWMGWISPN | 592 |
| | | NGETNYAQKFQDRVTMTRDTSISTAYMELSRLRSDDTAVYYCAREATIFGMLIVPF | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361395 | 12H11.013 | QVQLVQSGAEVKKPGASVKVSCKASGYTFTGYYIHWVRQAPGQGLEWMGWISP | 593 |
| | | NQGETNYAQKFQDRVTMTRDTSISTAYMELSSLRSDDTAVYYCAREATIFGMLIV | |
| | | PFDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361402 | 12H11.014 | QVQLVQSGAEVKKPGASVKVSCKASGYTFTGYYIHWVRQAPGQGLEWMGWISP | 594 |
| | | NQGETNYAQKFQDRVTMTRDTSISTAYMELSSLRSDDTAVYYCAREATIFGMLIV | |
| | | PFDYWGQGTLVTVSSASTKGPSVFPLAPSSKSTSGGTAALGCLVKDYFPEPVTVSW | |
| | | NSGALTSGVHTFPAVLQSSGLYSLSSVVTVPSSSLGTQTYICNVNHKPSNTKVDKK | |
| | | VEPKSCDKTHTCPPCPAPELLGGPSVFLFPPKPKDTLMISRTPEVTCVVVDVSHEDP | |
| | | EVKFNWYVDGVEVHNAKTKPCEEQYGSTYRCVSVLTVLHQDWLNGKEYKCKVS | |
| | | NKALPAPIEKTISKAKGQPREPQVYTLPPSREEMTKNQVSLTCLVKGFYPSDIAVE | |
| | | WESNGQPENNYKTTPPVLDSDGSFFLYSKLTVDKSRWQQGNVFSCSVMHEALHN | |
| | | HYTQKSLSLSPGK | |
| iPS:361406 | 2C2.005.001 | QMQVVQSGAEVKKPGASVKVSCKASGYTFTGYYMHWVRQAPGQGLEWMGWIN | 595 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361412 | 2C2.005.002 | QMQVVQSGAEVKKPGASVKVSCKASGYTFTGYYMHWVRQAPGQGLEWMGWIN | 596 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361418 | 2C2.005.003 | QMQVVQSGAEVKKPGASVKVSCKASGYTFTGYYMHWVRQAPGQGLEWMGWIN | 597 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361424 | 2C2.005.004 | QMQVVQSGAEVKKPGASVKVSCKASGYTFTGYYMHWVRQAPGQGLEWMGWIN | 598 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361430 | 2C2.005.005 | QMQVVQSGAEVKKPGASVKVSCKASGYTFTGYYMHWVRQAPGQGLEWMGWIN | 599 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361436 | 2C2.005.006 | QMQVVQSGAEVKKPGASVKVSCKASGYTFTGYYMHWVRQAPGQGLEWMGWIN | 600 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361442 | 2C2.005.007 | QMQVVQSGAEVKKPGASVKVSCKASGYTFTGYYMHWVRQAPGQGLEWMGWIN | 601 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361448 | 2C2.005.008 | QMQVVQSGAEVKKPGASVKVSCKASGYTFTGYYMHWVRQAPGQGLEWMGWIN | 602 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361454 | 2C2.005.009 | QMQVVQSGAEVKKPGASVKVSCKASGYTFTGYYMHWVRQAPGQGLEWMGWIN | 603 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361460 | 2C2.005.010 | QMQVVQSGAEVKKPGASVKVSCKASGYTFTGYYMHWVRQAPGQGLEWMGWIN | 604 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361466 | 2C2.005.011 | QMQVVQSGAEVKKPGASVKVSCKASGYTFTGYYMHWVRQAPGQGLEWMGWIN | 605 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361472 | 2C2.005.012 | QMQVVQSGAEVKKPGASVKVSCKASGYTFTGYYMHWVRQAPGQGLEWMGWIN | 606 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361478 | 2C2.005.013 | QMQVVQSGAEVKKPGASVKVSCKASGYTFTGYYMHWVRQAPGQGLEWMGWIN | 607 |
| | | PNSGGTNYAQKFQGRVTMTRDTSISTAYMELSSLRSDDTAVYYCARGGDYVFGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361485 | 2C2.005.014 | QMQVVQSGAEVKKPGASVKVSCKASGYTFTGYYMHWVRQAPGQGLEWMGWIN | 608 |
| | | PNSGGTNYAQKFQGRVTMTRDTSISTAYMELSRLRSDDTAVYYCARGGDYVFGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:361845 | 18F2.013 | QVQLVESGGGVVQPGRSLRLSCAASGFTFSNFGMHWVRQAPGKGLEWVAVIWY | 609 |
| | | DASNENYADAVKGRFTISRDNSKNTLYLQMNSLRAEDTAVYYCARDRTIFGVVLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361851 | 6H1.009 | QVQLVESGGGVVQPGRSLRLSCAASGFTFSYFGMHWVRQAPGKGLEWVAVIWY | 610 |
| | | DASNKYYADAVKGRFTISRDNSKNTLYLQMNSLRAEDTAVYYCARDRTIFGVLLG | |
| | | DYWGQGTLVTVSSASTKGPSVFPLAPSSKSTSGGTAALGCLVKDYFPEPVTVSWN | |
| | | SGALTSGVHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVVDVSHEDPE | |
| | | VKFNWYVDGVEVHNAKTKPCEEQYGSTYRCVSVLTVLHQDWLNGKEYKCKVSN | |
| | | KALPAPIEKTISKAKGQPREPQVYTLPPSREEMTKNQVSLTCLVKGFYPSDIAVEWE | |
| | | SNGQPENNYKTTPPVLDSDGSFFLYSKLTVDKSRWQQGNVFSCSVMHEALHNHY | |
| | | TQKSLSLSPGK | |
| iPS:361855 | 2C2.005.015 | QMQVVQSGAEVKKPGASVKVSCKASGYTFTGYYMHWVRQAPGQGLEWMGWIN | 611 |
| | | PNSGGTNYAQKFQGRVTMTRDTSISTAYMELSRLRSDDTAVYYCARGGDYVWGT | |
| | | YRPHYYYGMDVWGQGTTVTVSSASTKGPSVFPLAPSSKSTSGGTAALGCLVKDYF | |
| | | PEPVTVSWNSGALTSGVHTFPAVLQSSGLYSLSSVVTVPSSSLGTQTYICNVNHKPS | |
| | | NTKVDKKVEPKSCDKTHTCPPCPAPELLGGPSVFLFPPKPKDTLMISRTPEVTCVV | |
| | | VDVSHEDPEVKFNWYVDGVEVHNAKTKPCEEQYGSTYRCVSVLTVLHQDWLNG | |
| | | KEYKCKVSNKALPAPIEKTISKAKGQPREPQVYTLPPSREEMTKNQVSLTCLVKGF | |
| | | YPSDIAVEWESNGQPENNYKTTPPVLDSDGSFFLYSKLTVDKSRWQQGNVFSCSV | |
| | | MHEALHNHYTQKSLSLSPGK | |
| iPS:336067 | 5G12.006 | QVQLVESGGGVVQPGRSLRLSCTASGFTFSSYGIHWVRQAPGKGLEWVAVIWYD | 612 |
| | | GSNKFHADSVKGRFTISRDNSKNTLYLQMNSLRAEDSAMYFCARGKVAGMPEAF | |
| | | EIWGQGTKVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:336169 | 17B11.002 | QMQLVQSGAEVKKPGASVKVSCKASGYTFTSYDINWVRQATGQGLEWMGWMN | 613 |
| | | PNSGNTGYAQKFQGRVTMTRDTSISTAYMELSSLRSQDTAVYYCARGGDYVWGS | |
| | | YRPYYYYYGMDVWGQGTTVTVSSASTKGPSVFPLAPSSKSTSGGTAALGCLVKD | |
| | | YFPEPVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRTPEVTC | |
| | | VVVDVSHEDPEVKFNWYVDGVEVHNAKTKPCEEQYGSTYRCVSVLTVLHQDWL | |
| | | NGKEYKCKVSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRWQQGNVFSC | |
| | | SVMHEALHNHYTQKSLSLSPGK | |
| iPS:361127 | 5G12.006.001 | QVQLVESGGGVVQPGRSLRLSCAASGFTFSSYGIHWVRQAPGKGLEWVAVIWYD | 614 |
| | | ASNKFHADAVKGRFTISRDNSKNTLYLQMNSLRAEDSAMYFCARGKVAGMPEAF | |
| | | EIWGQGTKVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:361136 | 5G12.006.002 | QVQLVESGGGVVQPGRSLRLSCTASGFTFSSYGIHWVRQAPGKGLEWVAVIWYD | 615 |
| | | ASNKFHADAVKGRFTISRDNSKNTLYLQMNSLRAEDSAMYFCARGKVAGMPEAF | |
| | | EIWGQGTKVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:361140 | 5G12.006.003 | QVQLVESGGGVVQPGRSLRLSCAASGFTFSSYGIHWVRQAPGKGLEWVAVIWYD | 616 |
| | | ASNKFHADAVKGRFTISRDNSKNTLYLQMNSLRAEDSAMYFCARGKVAGMPEAF | |
| | | EIWGQGTKVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:359919 | 5G12.006.004 | QVQLVESGGGVVQPGRSLRLSCAASGFTFSSYGIHWVRQAPGKGLEWVAVIWYD | 617 |
| | | ASNKFHADAVKGRFTISRDNSKNTLYLQMNSLRAEDSAMYFCARGKVAGMPEAF | |
| | | EIWGQGTLVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:361144 | 5G12.006.005 | QVQLVESGGGVVQPGRSLRLSCAASGFTFSSYGIHWVRQAPGKGLEWVAVIWYD | 618 |
| | | ASNKFHADSVKGRFTISRDNSKNTLYLQMNSLRAEDSAMYFCARGKVAGMPEAF | |
| | | EIWGQGTKVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:361151 | 5G12.006.006 | QVQLVESGGGVVQPGRSLRLSCAASGFTFSSYGIHWVRQAPGKGLEWVAVIWYD | 619 |
| | | GSNKFHADAVKGRFTISRDNSKNTLYLQMNSLRAEDSAMYFCARGKVAGMPEAF | |
| | | EIWGQGTKVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |
| iPS:361491 | 17B11.002.001 | QMQLVQSGAEVKKPGASVKVSCKASGYTFTSYDINWVRQATGQGLEWMGWMN | 620 |
| | | PNSGGTGYAQKFQGRVTMTRDTSISTAYMELSSLRSQDTAVYYCARGGDYVWGS | |
| | | YRPYYYYYGMDVWGQGTTVTVSSASTKGPSVFPLAPSSKSTSGGTAALGCLVKD | |
| | | YFPEPVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRTPEVTC | |
| | | VVVDVSHEDPEVKFNWYVDGVEVHNAKTKPCEEQYGSTYRCVSVLTVLHQDWL | |
| | | NGKEYKCKVSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRWQQGNVFSC | |
| | | SVMHEALHNHYTQKSLSLSPGK | |
| iPS:361499 | 17B11.002.002 | QMQLVQSGAEVKKPGASVKVSCKASGYTFTSYDINWVRQATGQGLEWMGWMN | 621 |
| | | PNSGNTGYAQKFQGRVTMTRDTSISTAYMELSSLRSQDTAVYYCARGGDYVWGS | |
| | | YRPYYYYYGMDVWGQGTTVTVSSASTKGPSVFPLAPSSKSTSGGTAALGCLVKD | |
| | | YFPEPVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRTPEVTC | |
| | | VVVDVSHEDPEVKFNWYVDGVEVHNAKTKPCEEQYGSTYRCVSVLTVLHQDWL | |
| | | NGKEYKCKVSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRWQQGNVFSC | |
| | | SVMHEALHNHYTQKSLSLSPGK | |
| iPS:361503 | 17B11.002.003 | QMQLVQSGAEVKKPGASVKVSCKASGYTFTSYDINWVRQATGQGLEWMGWMN | 622 |
| | | PNSGGTGYAQKFQGRVTMTRDTSISTAYMELSSLRSQDTAVYYCARGGDYVWGS | |
| | | YRPYYYYYGMDVWGQGTTVTVSSASTKGPSVFPLAPSSKSTSGGTAALGCLVKD | |
| | | YFPEPVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRTPEVTC | |
| | | VVVDVSHEDPEVKFNWYVDGVEVHNAKTKPCEEQYGSTYRCVSVLTVLHQDWL | |
| | | NGKEYKCKVSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRWQQGNVFSC | |
| | | SVMHEALHNHYTQKSLSLSPGK | |
| iPS:361507 | 17B11.002.004 | QMQLVQSGAEVKKPGASVKVSCKASGYTFTSYDINWVRQAPGQGLEWMGWMN | 623 |
| | | PNSGGTGYAQKFQGRVTMTRDTSISTAYMELSSLRSDDTAVYYCARGGDYVWGS | |
| | | YRPYYYYYGMDVWGQGTTVTVSSASTKGPSVFPLAPSSKSTSGGTAALGCLVKD | |
| | | YFPEPVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRTPEVTC | |
| | | VVVDVSHEDPEVKFNWYVDGVEVHNAKTKPCEEQYGSTYRCVSVLTVLHQDWL | |
| | | NGKEYKCKVSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRWQQGNVFSC | |
| | | SVMHEALHNHYTQKSLSLSPGK | |
| iPS:361514 | 17B11.002.005 | QMQLVQSGAEVKKPGASVKVSCKASGYTFTSYDINWVRQATGQGLEWMGWMN | 624 |
| | | PNSGGTGYAQKFQGRVTMTRDTSISTAYMELSSLRSQDTAVYYCARGGDYVWGS | |
| | | YRPYYYYYGMDVWGQGTTVTVSSASTKGPSVFPLAPSSKSTSGGTAALGCLVKD | |
| | | YFPEPVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRTPEVTC | |
| | | VVVDVSHEDPEVKFNWYVDGVEVHNAKTKPCEEQYGSTYRCVSVLTVLHQDWL | |
| | | NGKEYKCKVSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRWQQGNVFSC | |
| | | SVMHEALHNHYTQKSLSLSPGK | |
| iPS:360582 | 17B11.002.006 | QMQLVQSGAEVKKPGASVKVSCKASGYTFTSYDINWVRQAPGQGLEWMGWMN | 625 |
| | | PNSGNTGYAQKFQGRVTMTRDTSISTAYMELSSLRSDDTAVYYCARGGDYVWGS | |
| | | YRPYYYYYGMDVWGQGTTVTVSSASTKGPSVFPLAPSSKSTSGGTAALGCLVKD | |
| | | YFPEPVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRTPEVTC | |
| | | VVVDVSHEDPEVKFNWYVDGVEVHNAKTKPCEEQYGSTYRCVSVLTVLHQDWL | |
| | | NGKEYKCKVSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRWQQGNVFSC | |
| | | SVMHEALHNHYTQKSLSLSPGK | |
| iPS:361518 | 17B11.002.007 | QMQLVQSGAEVKKPGASVKVSCKASGYTFTSYDINWVRQATGQGLEWMGWMN | 626 |
| | | PNSGGTGYAQKFQGRVTMTRDTSISTAYMELSSLRSQDTAVYYCARGGDYVFGS | |
| | | YRPYYYYYGMDVWGQGTTVTVSSASTKGPSVFPLAPSSKSTSGGTAALGCLVKD | |
| | | YFPEPVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRTPEVTC | |
| | | VVVDVSHEDPEVKFNWYVDGVEVHNAKTKPCEEQYGSTYRCVSVLTVLHQDWL | |
| | | NGKEYKCKVSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRWQQGNVFSC | |
| | | SVMHEALHNHYTQKSLSLSPGK | |
| iPS:360570 | 17B11.002.008 | QMQLVQSGAEVKKPGASVKVSCKASGYTFTSYDINWVRQAPGQGLEWMGWMN | 627 |
| | | PNSGGTGYAQKFQGRVTMTRDTSISTAYMELSSLRSDDTAVYYCARGGDYVFGS | |
| | | YRPYYYYYGMDVWGQGTTVTVSSASTKGPSVFPLAPSSKSTSGGTAALGCLVKD | |
| | | YFPEPVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRTPEVTC | |
| | | VVVDVSHEDPEVKFNWYVDGVEVHNAKTKPCEEQYGSTYRCVSVLTVLHQDWL | |
| | | NGKEYKCKVSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRWQQGNVFSC | |
| | | SVMHEALHNHYTQKSLSLSPGK | |
| iPS:362051 | 5G12.005.001 | QVQLVESGGGVVQPGRSLRLSCAASGFTFSSYGIHWVRQAPGKGLEWVAVIWYD | 628 |
| | | ASNKFHADAVKGRFTISRDNSKNTLYLQMNSLRAEDSAMYFCARGKVAGMPEAF | |
| | | EIWGQGTLVTVSSASTKGPSVFPLAPSSKSTSGGTAALGCLVKDYFPEPVTVSWNS | |
| | | GALTSGVHTFPAVLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTCVVVDVSHEDPEV | |
| | | KFNWYVDGVEVHNAKTKPCEEQYGSTYRCVSVLTVLHQDWLNGKEYKCKVSNK | |
| | | ALPAPIEKTISKAKGQPREPQVYTLPPSREEMTKNQVSLTCLVKGFYPSDIAVEWES | |
| | | NGQPENNYKTTPPVLDSDGSFFLYSKLTVDKSRWQQGNVFSCSVMHEALHNHYT | |
| | | QKSLSLSPGK | |

TABLE 16
Light Chain (LC) Amino Acid Sequences of Example Anti-GIPR Antibodies with Cysteine Modifications

| Description | LC | SEQ ID NO: |
|---|---|---|
| aGIPR 2G10 | EIVMTQSPATLSVSPGERATLSCRASQSVSSNLAWYQQKPGQAPRLLIYGAATRATGIPARVSG | 388 |
| SEFL2 E384C | SGSGTEFTLTISSLQSEDFAVYYCQQYNNWPLTFGGGTKVEIKRTVAAPSVFIFPPSDEQLKSGT | |
| | ASVVCLLNNFYPREAKVQWKVDNALQSGNSQESVTEQDSKDSTYSLSSTLTLSKADYEKHKV | |
| | YACEVTHQGLSSPVTKSFNRGEC | |
| aGIPR 5G12 | DIQLTQSPSSLSASVGDRVTITCRASQTISRFLNWYQQKPGKAPELLIYVASSLQSGVPSRFSGS | 455 |
| SEFL2 E384C | GSGTDFTLTISSLQPEDFATYYCQQSYSTLISFGQGTKLEITRTVAAPSVFIFPPSDEQLKSGTAS | |
| | VVCLLNNFYPREAKVQWKVDNALQSGNSQESVTEQDSKDSTYSLSSTLTLSKADYEKHKVYA | |
| | CEVTHQGLSSPVTKSFNRGEC | |
| aGIPR 2G10.007 | EIVMTQSPATLSVSPGERATLSCRASQSVSSNLAWYQQKPGQAPRLLIYGAATRATGIPARFSG | 389 |
| SEFL2 E384C | SGSGTEFTLTISSLEPEDFAVYYCQQYNNFPLTFGGGTKVEIKRTVAAPSVFIFPPSDEQLKSGT | |
| | ASVVCLLNNFYPREAKVQWKVDNALQSGNSQESVTEQDSKDSTYSLSSTLTLSKADYEKHKV | |
| | YACEVTHQGLSSPVTKSFNRGEC | |
| aGIPR 5G12 | DIQLTQSPSSLSASVGDRVTITCRASQTISRFLNWYQQKPGKAPELLIYVASSLQSGVPSRFSGS | 455 |
| SEFL2 T487C | GSGTDFTLTISSLQPEDFATYYCQQSYSTLISFGQGTKLEITRTVAAPSVFIFPPSDEQLKSGTAS | |
| | VVCLLNNFYPREAKVQWKVDNALQSGNSQESVTEQDSKDSTYSLSSTLTLSKADYEKHKVYA | |
| | CEVTHQGLSSPVTKSFNRGEC | |
| aGIPR 5G12 | DIQLTQSPSSLSASVGDRVTITCRASQTISRFLNWYQQKPGKAPELLIYVASSLQSGVPSRFSGS | 1575 |
| SEFL2 D88C | GSGTCFTLTISSLQPEDFATYYCQQSYSTLISFGQGTKLEITRTVAAPSVFIFPPSDEQLKSGTAS | |
| | VVCLLNNFYPREAKVQWKVDNALQSGNSQESVTEQDSKDSTYSLSSTLTLSKADYEKHKVYA | |
| | CEVTHQGLSSPVTKSFNRGEC | |

TABLE 17
Heavy Chain (HC) Amino Acid Sequences of Example Anti-GIPR Antibodies with Cysteine Modifications

| Description | HC | SEQ ID NO: |
|---|---|---|
| aGIPR 2G10 | QVQLVESGGGVVQPGRSLRLSCAASGFTFSNYGMHWVRQAPGEGLEWVAAIWFDASDKYY | 1571 |
| SEFL2 E384C | ADAVKGRFTISRDNSKNTLYLQMNSLRAEDTAVYYCARDQAIFGVVPDYWGQGTLVTVSSAS | |
| | TKGPSVFPLAPSSKSTSGGTAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPPCPAPELLGGPSVFLFPPKP | |
| | KDTLMISRTPEVTCVVVDVSHEDPCVKFNWYVDGVEVHNAKTKPCEEQYGSTYRCVSVLTV | |
| | LHQDWLNGKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVFSCSVMHEAL | |
| | HNHYTQKSLSLSPGK | |
| aGIPR 5G12 | QVQLVESGGGVVQPGRSLRLSCTASGFTFSSYGIHWVRQAPGKGLEWVAVIWYDGSNKFHA | 1572 |
| SEFL2 E384C | DSVKGRFTISRDNSKNTLYLQMNSLRAEDSAMYFCARGKVAGMPEAFEIWGQGTKVTVSSAS | |
| | TKGPSVFPLAPSSKSTSGGTAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPPCPAPELLGGPSVFLFPPKP | |
| | KDTLMISRTPEVTCVVVDVSHEDPCVKFNWYVDGVEVHNAKTKPCEEQYGSTYRCVSVLTV | |
| | LHQDWLNGKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVFSCSVMHEAL | |
| | HNHYTQKSLSLSPGK | |
| aGIPR 2G10.007 | QVQLVESGGGVVQPGRSLRLSCAASGFTFSNYGMHWVRQAPGKGLEWVAAIWFDASDKYY | 1573 |
| SEFL2 E384C | ADAVKGRFTISRDNSKNTLYLQMNSLRAEDTAVYYCARDQAIFGVVPDYWGQGTLVTVSSAS | |
| | TKGPSVFPLAPSSKSTSGGTAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPPCPAPELLGGPSVFLFPPKP | |
| | KDTLMISRTPEVTCVVVDVSHEDPCVKFNWYVDGVEVHNAKTKPCEEQYGSTYRCVSVLTV | |
| | LHQDWLNGKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVFSCSVMHEAL | |
| | HNHYTQKSLSLSPGK | |
| aGIPR 5G12 | QVQLVESGGGVVQPGRSLRLSCTASGFTFSSYGIHWVRQAPGKGLEWVAVIWYDGSNKFHA | 1574 |
| SEFL2 T487C | DSVKGRFTISRDNSKNTLYLQMNSLRAEDSAMYFCARGKVAGMPEAFEIWGQGTKVTVSSAS | |
| | TKGPSVFPLAPSSKSTSGGTAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPPCPAPELLGGPSVFLFPPKP | |
| | KDTLMISRTPEVTCVVVDVSHEDPEVKFNWYVDGVEVHNAKTKPCEEQYGSTYRCVSVLTVL | |
| | HQDWLNGKEYKCKVSNKALPAPIEKTISKAKGQPREPQVYTLPPSREEMCKNQVSLTCLVKG | |
| | FYPSDIAVEWESNGQPENNYKTTPPVLDSDGSFFLYSKLTVDKSRWQQGNVFSCSVMHEALH | |
| | NHYTQKSLSLSPGK | |
| aGIPR 5G12 | QVQLVESGGGVVQPGRSLRLSCTASGFTFSSYGIHWVRQAPGKGLEWVAVIWYDGSNKFHA | 612 |
| SEFL2 D88C | DSVKGRFTISRDNSKNTLYLQMNSLRAEDSAMYFCARGKVAGMPEAFEIWGQGTKVTVSSAS | |
| | TKGPSVFPLAPSSKSTSGGTAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPPCPAPELLGGPSVFLFPPKP | |
| | KDTLMISRTPEVTCVVVDVSHEDPEVKFNWYVDGVEVHNAKTKPCEEQYGSTYRCVSVLTVL | |
| | HQDWLNGKEYKCKVSNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGNVFSCSVMHEALHN | |
| | HYTQKSLSLSPGK | |

In some embodiments, the antigen-binding protein (e.g., the anti-GIPR antibody) comprises a light chain variable region and a heavy chain variable region as listed in one of the rows for one of the antibodies listed in Tables 9, 10, and 11. In some embodiments, the antigen-binding protein (e.g., the anti-GIPR antibody) comprises two light chain variable region and two identical heavy chain variable regions from one of the antibodies listed in Tables 9, 10, and 11. In some embodiments, the antigen-binding protein (e.g., the anti-GIPR antibody) comprises a light chain variable region and a heavy chain variable region as listed in one of the rows for one of the antibodies listed in Tables 9, 10, and 11, except that one or both of the light chain variable region and the heavy chain variable region differs from the sequence specified in Table 10 (VL) or Table 11 (VH) at 1, 2, 3, 4, 5, 6, 7, 8, 9, 10, 11, 12, 13, 14 or 15 amino acid residues (combined), wherein each such sequence difference is independently either a single amino acid deletion, insertion, or substitution, with the deletions, insertions and/or substitutions resulting in no more than 1, 2, 3, 4, 5, 6, 7, 8, 9, 10, 11, 12, 13, 14 or 15 amino acid changes (combined) relative to the light chain variable region and heavy chain variable region sequences specified in Table 10 (VL) or Table 11 (VH). In some embodiments, the antigen-binding protein (e.g., the anti-GIPR antibody) comprises a variable region sequence from Table 10 or Table 11, but with the N-terminal methionine deleted.

In some embodiments, the antigen-binding protein (e.g., the anti-GIPR antibody) comprises a light chain variable region and a heavy chain variable region as listed in one of the rows for one of the antibodies listed in Tables 9, 10, or 11, except that one or both of the variable regions differs from the amino acid sequence specified in Table 10 (VL) or Table 11 (VH) in that the heavy chain variable region and/or light chain variable region comprises or consists of a sequence of amino acids that has at least 70%, 75%, 80%, 85%, 90%, 95%, 96%, 97%, 98%, or 99% sequence identity to the amino acid sequences of the heavy chain variable region or light chain variable domain region as specified in Table 11 or Table 10, respectively.

In some embodiments, the antigen-binding protein consists just of a light chain variable region or a heavy chain variable region from an antibody listed in Table 9.

In some embodiments, the antigen-binding protein comprises two or more of the same heavy chain variable region or two or more of the same light chain variable region from those listed in Tables 10 and 11. Such domain antibodies can be fused together or joined via a linker moiety, such as a linker moiety described below. The domain antibodies can also be fused or linked to one or more molecules to extend the half-life (such as, e.g., PEG or albumin).

Other antigen-binding proteins that are provided are variants of antibodies formed by combination of the heavy and light chains shown in Tables 10 and 11 and comprise light and/or heavy chains that each have at least 70%, at least 75%, at least 80%, at least 85%, at least 90%, at least 95%, at least 96%, at least 97%, at least 98%, or at least 99% identity to the amino acid sequences of these chains. In some embodiments, such antibody variants comprise at least one heavy chain and at least one light chain. In some embodiments, the antibody variant comprises two identical light chains and two identical heavy chains.

In some embodiments, the antigen-binding protein is a human antibody comprising a sequence as set forth in Table 9 and is of the IgG₁-, IgG₂- IgG₃-, or IgG₄-type.

In some embodiments, the antigen-binding protein (e.g., the anti-GIPR antibody) comprises a light chain variable region comprising an amino acid sequence selected from the group consisting of SEQ ID NOs: 1-157 and a heavy chain variable region comprising an amino acid sequence selected from the group consisting of SEQ ID NOs: 158-314.

In some embodiments, the antigen-binding protein is an antibody comprising a light chain variable region comprising an amino acid sequence selected from the group consisting of SEQ ID NOs: 1-157 and a heavy chain variable region comprising an amino acid sequence selected from the group consisting of SEQ ID NOs: 158-314, wherein the antibody comprises at least one cysteine conjugation site at a position selected from the group consisting of 88 (e.g., D88) of the light chain, 384 (e.g., E384) of the heavy chain, and 487 (e.g., T487) of the heavy chain, all according to AHo numbering.

In some embodiments, the antigen-binding protein (e.g., the anti-GIPR antibody) comprises a light chain variable region and a heavy chain variable region, wherein the light chain variable region and the heavy chain variable region comprise amino acid sequences selected from:
i. SEQ ID NO: 1 and SEQ ID NO: 158, respectively;
ii. SEQ ID NO: 2 and SEQ ID NO: 159, respectively;
iii. SEQ ID NO: 3 and SEQ ID NO: 160, respectively;
iv. SEQ ID NO: 4 and SEQ ID NO: 161, respectively;
v. SEQ ID NO: 5 and SEQ ID NO: 162, respectively;
vi. SEQ ID NO: 6 and SEQ ID NO: 163, respectively;
vii. SEQ ID NO: 7 and SEQ ID NO: 164, respectively;
viii. SEQ ID NO: 8 and SEQ ID NO: 165, respectively;
ix. SEQ ID NO: 9 and SEQ ID NO: 166, respectively;
x. SEQ ID NO: 10 and SEQ ID NO: 167, respectively;
xi. SEQ ID NO: 11 and SEQ ID NO: 168, respectively;
xii. SEQ ID NO: 12 and SEQ ID NO: 169, respectively;
xiii. SEQ ID NO: 13 and SEQ ID NO: 170, respectively;
xiv. SEQ ID NO: 14 and SEQ ID NO: 171, respectively;
xv. SEQ ID NO: 15 and SEQ ID NO: 172, respectively;
xvi. SEQ ID NO: 16 and SEQ ID NO: 173, respectively;
xvii. SEQ ID NO: 17 and SEQ ID NO: 174, respectively;
xviii. SEQ ID NO: 18 and SEQ ID NO: 175, respectively;
xix. SEQ ID NO: 19 and SEQ ID NO: 176, respectively;
xx. SEQ ID NO: 20 and SEQ ID NO: 177, respectively;
xxi. SEQ ID NO: 21 and SEQ ID NO: 178, respectively;
xxii. SEQ ID NO: 22 and SEQ ID NO: 179, respectively;
xxiii. SEQ ID NO: 23 and SEQ ID NO: 180, respectively;
xxiv. SEQ ID NO: 24 and SEQ ID NO: 181, respectively;
xxv. SEQ ID NO: 25 and SEQ ID NO: 182, respectively;
xxvi. SEQ ID NO: 26 and SEQ ID NO: 183, respectively;
xxvii. SEQ ID NO: 27 and SEQ ID NO: 184, respectively;
xxviii. SEQ ID NO: 28 and SEQ ID NO: 185, respectively;
xxix. SEQ ID NO: 29 and SEQ ID NO: 186, respectively;
xxx. SEQ ID NO: 30 and SEQ ID NO: 187, respectively;
xxxi. SEQ ID NO: 31 and SEQ ID NO: 188, respectively;
xxxii. SEQ ID NO: 32 and SEQ ID NO: 189, respectively;
xxxiii. SEQ ID NO: 33 and SEQ ID NO: 190, respectively;
xxxiv. SEQ ID NO: 34 and SEQ ID NO: 191, respectively;
xxxv. SEQ ID NO: 35 and SEQ ID NO: 192, respectively;
xxxvi. SEQ ID NO: 36 and SEQ ID NO: 193, respectively;
xxxvii. SEQ ID NO: 37 and SEQ ID NO: 194, respectively;
xxxviii. SEQ ID NO: 38 and SEQ ID NO: 195, respectively;
xxxix. SEQ ID NO: 39 and SEQ ID NO: 196, respectively;
xl. SEQ ID NO: 40 and SEQ ID NO: 197, respectively;
xli. SEQ ID NO: 41 and SEQ ID NO: 198, respectively;
xlii. SEQ ID NO: 42 and SEQ ID NO: 199, respectively;
xliii. SEQ ID NO: 43 and SEQ ID NO: 200, respectively;
xliv. SEQ ID NO: 44 and SEQ ID NO: 201, respectively;
xlv. SEQ ID NO: 45 and SEQ ID NO: 202, respectively;
xlvi. SEQ ID NO: 46 and SEQ ID NO: 203, respectively;
xlvii. SEQ ID NO: 47 and SEQ ID NO: 204, respectively;
xlviii. SEQ ID NO: 48 and SEQ ID NO: 205, respectively;
xlix. SEQ ID NO: 49 and SEQ ID NO: 206, respectively;
l. SEQ ID NO: 50 and SEQ ID NO: 207, respectively;
li. SEQ ID NO: 51 and SEQ ID NO: 208, respectively;
lii. SEQ ID NO: 52 and SEQ ID NO: 209, respectively;
liii. SEQ ID NO: 53 and SEQ ID NO: 210, respectively;
liv. SEQ ID NO: 54 and SEQ ID NO: 211, respectively;
lv. SEQ ID NO: 55 and SEQ ID NO: 212, respectively;
lvi. SEQ ID NO: 56 and SEQ ID NO: 213, respectively;
lvii. SEQ ID NO: 57 and SEQ ID NO: 214, respectively;
lviii. SEQ ID NO: 58 and SEQ ID NO: 215, respectively;
lix. SEQ ID NO: 59 and SEQ ID NO: 216, respectively;
lx. SEQ ID NO: 60 and SEQ ID NO: 217, respectively;
lxi. SEQ ID NO: 61 and SEQ ID NO: 218, respectively;
lxii. SEQ ID NO: 62 and SEQ ID NO: 219, respectively;
lxiii. SEQ ID NO: 63 and SEQ ID NO: 220, respectively;
lxiv. SEQ ID NO: 64 and SEQ ID NO: 221, respectively;
lxv. SEQ ID NO: 65 and SEQ ID NO: 222, respectively;
lxvi. SEQ ID NO: 66 and SEQ ID NO: 223, respectively;
lxvii. SEQ ID NO: 67 and SEQ ID NO: 224, respectively;
lxviii. SEQ ID NO: 68 and SEQ ID NO: 225, respectively;
lxix. SEQ ID NO: 69 and SEQ ID NO: 226, respectively;
lxx. SEQ ID NO: 70 and SEQ ID NO: 227, respectively;
lxxi. SEQ ID NO: 71 and SEQ ID NO: 228, respectively;
lxxii. SEQ ID NO: 72 and SEQ ID NO: 229, respectively;
lxxiii. SEQ ID NO: 73 and SEQ ID NO: 230, respectively;
lxxiv. SEQ ID NO: 74 and SEQ ID NO: 231, respectively;
lxxv. SEQ ID NO: 75 and SEQ ID NO: 232, respectively;
lxxvi. SEQ ID NO: 76 and SEQ ID NO: 233, respectively;
lxxvii. SEQ ID NO: 77 and SEQ ID NO: 234, respectively;
lxxviii. SEQ ID NO: 78 and SEQ ID NO: 235, respectively;
lxxix. SEQ ID NO: 79 and SEQ ID NO: 236, respectively;
lxxx. SEQ ID NO: 80 and SEQ ID NO: 237, respectively;
lxxxi. SEQ ID NO: 81 and SEQ ID NO: 238, respectively;
lxxxii. SEQ ID NO: 82 and SEQ ID NO: 239, respectively;
lxxxiii. SEQ ID NO: 83 and SEQ ID NO: 240, respectively;
lxxxiv. SEQ ID NO: 84 and SEQ ID NO: 241, respectively;
lxxxv. SEQ ID NO: 85 and SEQ ID NO: 242, respectively;
lxxxvi. SEQ ID NO: 86 and SEQ ID NO: 243, respectively;
lxxxvii. SEQ ID NO: 87 and SEQ ID NO: 244, respectively;
lxxxviii. SEQ ID NO: 88 and SEQ ID NO: 245, respectively;
lxxxix. SEQ ID NO: 89 and SEQ ID NO: 246, respectively;
xc. SEQ ID NO: 90 and SEQ ID NO: 247, respectively;
xci. SEQ ID NO: 91 and SEQ ID NO: 248, respectively;
xcii. SEQ ID NO: 92 and SEQ ID NO: 249, respectively;
xciii. SEQ ID NO: 93 and SEQ ID NO: 250, respectively;
xciv. SEQ ID NO: 94 and SEQ ID NO: 251, respectively;
xcv. SEQ ID NO: 95 and SEQ ID NO: 252, respectively;
xcvi. SEQ ID NO: 96 and SEQ ID NO: 253, respectively;
xcvii. SEQ ID NO: 97 and SEQ ID NO: 254, respectively;
xcviii. SEQ ID NO: 98 and SEQ ID NO: 255, respectively;
xcix. SEQ ID NO: 99 and SEQ ID NO: 256, respectively;
c. SEQ ID NO: 100 and SEQ ID NO: 257, respectively;
ci. SEQ ID NO: 101 and SEQ ID NO: 258, respectively;
cii. SEQ ID NO: 102 and SEQ ID NO: 259, respectively;
ciii. SEQ ID NO: 103 and SEQ ID NO: 260, respectively;
civ. SEQ ID NO: 104 and SEQ ID NO: 261, respectively;
cv. SEQ ID NO: 105 and SEQ ID NO: 262, respectively;
cvi. SEQ ID NO: 106 and SEQ ID NO: 263, respectively;
cvii. SEQ ID NO: 107 and SEQ ID NO: 264, respectively;
cviii. SEQ ID NO: 108 and SEQ ID NO: 265, respectively;
cix. SEQ ID NO: 109 and SEQ ID NO: 266, respectively;
cx. SEQ ID NO: 110 and SEQ ID NO: 267, respectively;
cxi. SEQ ID NO: 111 and SEQ ID NO: 268, respectively;
cxii. SEQ ID NO: 112 and SEQ ID NO: 269, respectively;
cxiii. SEQ ID NO: 113 and SEQ ID NO: 270, respectively;
cxiv. SEQ ID NO: 114 and SEQ ID NO: 271, respectively;
cxv. SEQ ID NO: 115 and SEQ ID NO: 272, respectively;
cxvi. SEQ ID NO: 116 and SEQ ID NO: 273, respectively;
cxvii. SEQ ID NO: 117 and SEQ ID NO: 274, respectively;
cxviii. SEQ ID NO: 118 and SEQ ID NO: 275, respectively;
cxix. SEQ ID NO: 119 and SEQ ID NO: 276, respectively;
cxx. SEQ ID NO: 120 and SEQ ID NO: 277, respectively;
cxxi. SEQ ID NO: 121 and SEQ ID NO: 278, respectively;
cxxii. SEQ ID NO: 122 and SEQ ID NO: 279, respectively;
cxxiii. SEQ ID NO: 123 and SEQ ID NO: 280, respectively;
cxxiv. SEQ ID NO: 124 and SEQ ID NO: 281, respectively;
cxxv. SEQ ID NO: 125 and SEQ ID NO: 282, respectively;
cxxvi. SEQ ID NO: 126 and SEQ ID NO: 283, respectively;
cxxvii. SEQ ID NO: 127 and SEQ ID NO: 284, respectively;
cxxviii. SEQ ID NO: 128 and SEQ ID NO: 285, respectively;
cxxix. SEQ ID NO: 129 and SEQ ID NO: 286, respectively;
cxxx. SEQ ID NO: 130 and SEQ ID NO: 287, respectively;
cxxxi. SEQ ID NO: 131 and SEQ ID NO: 288, respectively;
cxxxii. SEQ ID NO: 132 and SEQ ID NO: 289, respectively;
cxxxiii. SEQ ID NO: 133 and SEQ ID NO: 290, respectively;
cxxxiv. SEQ ID NO: 134 and SEQ ID NO: 291, respectively;
cxxxv. SEQ ID NO: 135 and SEQ ID NO: 292, respectively;
cxxxvi. SEQ ID NO: 136 and SEQ ID NO: 293, respectively;
cxxxvii. SEQ ID NO: 137 and SEQ ID NO: 294, respectively;
cxxxviii. SEQ ID NO: 138 and SEQ ID NO: 295, respectively;
cxxxix. SEQ ID NO: 139 and SEQ ID NO: 296, respectively;
cxl. SEQ ID NO: 140 and SEQ ID NO: 297, respectively;
cxli. SEQ ID NO: 141 and SEQ ID NO: 298, respectively;
cxlii. SEQ ID NO: 142 and SEQ ID NO: 299, respectively;
cxliii. SEQ ID NO: 143 and SEQ ID NO: 300, respectively;
cxliv. SEQ ID NO: 144 and SEQ ID NO: 301, respectively;
cxlv. SEQ ID NO: 145 and SEQ ID NO: 302, respectively;
cxlvi. SEQ ID NO: 146 and SEQ ID NO: 303, respectively;
cxlvii. SEQ ID NO: 147 and SEQ ID NO: 304, respectively;
cxlviii. SEQ ID NO: 148 and SEQ ID NO: 305, respectively;
cxlix. SEQ ID NO: 149 and SEQ ID NO: 306, respectively;
cl. SEQ ID NO: 150 and SEQ ID NO: 307, respectively;
cli. SEQ ID NO: 151 and SEQ ID NO: 308, respectively;
clii. SEQ ID NO: 152 and SEQ ID NO: 309, respectively;
cliii. SEQ ID NO: 153 and SEQ ID NO: 310, respectively;
cliv. SEQ ID NO: 154 and SEQ ID NO: 311, respectively;
clv. SEQ ID NO: 155 and SEQ ID NO: 312, respectively;
clvi. SEQ ID NO: 156 and SEQ ID NO: 313, respectively; and
clvii. SEQ ID NO: 157 and SEQ ID NO: 314, respectively,
preferably wherein the light chain variable region and the heavy chain variable region comprise the amino acid sequences of SEQ ID NO: 74 and SEQ ID NO: 231, respectively.

In some embodiments, the antigen-binding protein is an antibody comprising at least one cysteine conjugation site at a position selected from the group consisting of 88 (e.g., D88) of the light chain, 384 (e.g., E384) of the heavy chain, and 487 (e.g., T487) of the heavy chain, all according to AHo numbering.

In some embodiments, the antigen-binding protein (e.g., the anti-GIPR antibody) is a polypeptide into which one or more CDRs are grafted, inserted, and/or joined. In some embodiments, the antigen-binding protein has 1, 2, 3, 4, 5, or 6 CDRs. An antigen-binding protein thus can have, for example, one heavy chain CDR1 (“CDRH1”), and/or one heavy chain CDR2 (“CDRH2”), and/or one heavy chain CDR3 (“CDRH3”), and/or one light chain CDR1 (“CDRL1”), and/or one light chain CDR2 (“CDRL2”), and/or one light chain CDR3 (“CDRL3”). In some embodiments, the antigen-binding protein comprises both a CDRH3 and a CDRL3. Specific light and heavy chain CDRs are identified in Tables 12 and 13, respectively.

Complementarity determining regions (CDRs) and framework regions (FR) of a given antibody may be identified using the system described by Kabat et al. in Sequences of Proteins of Immunological Interest, 5th Ed., US Dept. of Health and Human Services, PHS, NIH, NIH Publication no. 91-3242, 1991. Certain antibodies that are disclosed herein comprise one or more amino acid sequences that are identical or have substantial sequence identity to the amino acid sequences of one or more of the CDRs presented in Tables 12 and 13. The specific CDRs identified in Tables 12 and 13 are defined by Kabat.

The structure and properties of CDRs within a naturally occurring antibody have been described above. Briefly, in a traditional antibody, the CDRs are embedded within a framework in the heavy or light chain variable region where they constitute the regions responsible for antigen binding and recognition. A variable region comprises at least three heavy or light chain CDRs, see, supra (Kabat et al., 1991, Sequences of Proteins of Immunological Interest, Public Health Service N.I.H., Bethesda, MD; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991, supra; see also Chothia and Lesk, 1987, supra). The CDRs provided herein, however, may not only be used to define the antigen-binding domain of a traditional antibody structure, but may be embedded in a variety of other polypeptide structures, as described herein.

In some embodiments, the antigen-binding protein (e.g., an anti-GIPR antibody) comprises 1, 2, 3, 4, 5, or 6 variant forms of the CDRs listed in Tables 12 and 13, each having at least 80%, at least 85%, at least 90%, at least 95%, at least 96%, at least 97%, at least 98%, or at least 99% sequence identity to a CDR sequence listed in Table 12 or 13. In some embodiments, an antigen-binding protein (e.g., an anti-GIPR antibody) comprises 1, 2, 3, 4, 5, or 6 of the CDRs listed in Tables 12 and 13, each or collectively differing by no more than 1, 2, 3, 4 or 5 amino acids from the CDRs listed in these tables.

In some embodiments, the antigen-binding protein (e.g., the anti-GIPR antibody) comprises 1, 2, 3, 4, 5, or all 6 of the CDRs listed in one of the rows for any particular antibody listed in Table 9.

In some embodiments, the antigen-binding protein (e.g., the anti-GIPR antibody) comprises 1, 2, 3, 4, 5, or 6 variant forms of the CDRs listed in one of the rows for an antibody in Table 9, each CDR having at least 80%, at least 85%, at least 90%, at least 95%, at least 96%, at least 97%, at least 98%, or at least 99% sequence identity to a CDR sequence listed in Table 12 or 13.

In some embodiments, the antigen-binding protein (e.g., the anti-GIPR antibody) comprises 1, 2, 3, 4, 5, or 6 of the CDRs listed in one of the rows of Table 9, each differing by no more than 1, 2, 3, 4, or 5 amino acids from the CDRs listed in Table 9.

In some embodiments, the antigen-binding protein (e.g., the anti-GIPR antibody) comprises all six of the CDRs listed in a row of Table 9 and the total number of amino acid changes to the CDRs collectively is no more than 1, 2, 3, 4, or 5 amino acids.

In some embodiments, the antigen-binding protein (e.g., the anti-GIPR antibody) comprises a light chain variable (VL) region in which the full set of VL CDRs 1, 2, and 3 (combined) has at least 95% sequence identity to the VL CDRs 1, 2, and 3 of any one of SEQ ID NOs: 1-157. In some embodiments, the antigen-binding protein (e.g., the anti-GIPR antibody) comprises a light chain variable (VL) region in which the full set of VL CDRs 1, 2, and 3 (combined) has at least 95% sequence identity to the VL CDRs 1, 2, and 3 of any one of SEQ ID NOs: 1-157, wherein the VL CDRs 1, 2, and 3 are defined by any one of Kabat, Chothia, or IGMT. In some embodiments, the antigen-binding protein (e.g., the anti-GIPR antibody) comprises light chain variable (VL) region in which the full set of VL CDRs 1, 2, and 3 (combined) has at least 95% sequence identity to the VL CDRs 1, 2, and 3 of any one of SEQ ID NOs: 1-157, wherein the VL CDRs 1, 2, and 3 are defined by Kabat. In some embodiments, the antigen-binding protein (e.g., the anti-GIPR antibody) comprises a light chain variable (VL) region in which the full set of VL CDRs 1, 2, and 3 (combined) has at least 95% sequence identity to the VL CDRs 1, 2, and 3 of any one of SEQ ID NOs: 1-157, wherein the VL CDRs 1, 2, and 3 are defined by Chothia. In some embodiments, the antigen-binding protein (e.g., the anti-GIPR antibody) comprises a light chain variable (VL) region in which the full set of VL CDRs 1, 2, and 3 (combined) has at least 95% sequence identity to the VL CDRs 1, 2, and 3 of any one of SEQ ID NOs: 1-157, wherein the VL CDRs 1, 2, and 3 are defined by IGMT.

In some embodiments, the antigen-binding protein (e.g., the anti-GIPR antibody) comprises a light chain variable (VL) region in which the full set of VL CDRs 1, 2, and 3 (combined) is identical to the VL CDRs 1, 2, and 3 of any one of SEQ ID NOs: 1-157. In some embodiments, the antigen-binding protein (e.g., the anti-GIPR antibody) comprises a light chain variable (VL) region in which the full set of VL CDRs 1, 2, and 3 (combined) is identical to the VL CDRs 1, 2, and 3 of any one of SEQ ID NOs: 1-157, wherein the VL CDRs 1, 2, and 3 are defined by any one of Kabat, Chothia, or IGMT. In some embodiments, the antigen-binding protein (e.g., the anti-GIPR antibody) comprises light chain variable (VL) region in which the full set of VL CDRs 1, 2, and 3 (combined) is identical to the VL CDRs 1, 2, and 3 of any one of SEQ ID NOs: 1-157, wherein the VL CDRs 1, 2, and 3 are defined by Kabat. In some embodiments, the antigen-binding protein (e.g., the anti-GIPR antibody) comprises a light chain variable (VL) region in which the full set of VL CDRs 1, 2, and 3 (combined) is identical to the VL CDRs 1, 2, and 3 of any one of SEQ ID NOs: 1-157, wherein the VL CDRs 1, 2, and 3 are defined by Chothia. In some embodiments, the antigen-binding protein (e.g., the anti-GIPR antibody) comprises a light chain variable (VL) region in which the full set of VL CDRs 1, 2, and 3 (combined) is identical to the VL CDRs 1, 2, and 3 of any one of SEQ ID NOs: 1-157, wherein the VL CDRs 1, 2, and 3 are defined by IGMT.

In some embodiments, the antigen-binding protein (e.g., the anti-GIPR antibody) comprises a heavy chain variable (VH) region in which the full set of VH CDRs 1, 2, and 3 (combined) has at least 95% sequence identity to the VH CDRs 1, 2, and 3 of any one of SEQ ID NOs: 158-314. In some embodiments, the antigen-binding protein (e.g., the anti-GIPR antibody) comprises a heavy chain variable (VH) region in which the full set of VH CDRs 1, 2, and 3 (combined) has at least 95% sequence identity to the VH CDRs 1, 2, and 3 of any one of SEQ ID NOs: 158-314, wherein the VH CDRs 1, 2, and 3 are defined by any one of Kabat, Chothia, or IGMT. In some embodiments, the antigen-binding protein (e.g., the anti-GIPR antibody) comprises a heavy chain variable (VH) region in which the full set of VH CDRs 1, 2, and 3 (combined) has at least 95% sequence identity to the VH CDRs 1, 2, and 3 of any one of SEQ ID NOs: 158-314, wherein the VH CDRs 1, 2, and 3 are defined by Kabat. In some embodiments, the antigen-binding protein (e.g., the anti-GIPR antibody) comprises a heavy chain variable (VH) region in which the full set of VH CDRs 1, 2, and 3 (combined) has at least 95% sequence identity to the VH CDRs 1, 2, and 3 of any one of SEQ ID NOs: 158-314, wherein the VH CDRs 1, 2, and 3 are defined by Chothia. In some embodiments, the antigen-binding protein (e.g., the anti-GIPR antibody) comprises a heavy chain variable (VH) region in which the full set of VH CDRs 1, 2, and 3 (combined) has at least 95% sequence identity to the VH CDRs 1, 2, and 3 of any one of SEQ ID NOs: 158-314, wherein the VH CDRs 1, 2, and 3 are defined by IGMT.

In some embodiments, the antigen-binding protein (e.g., the anti-GIPR antibody) comprises a heavy chain variable (VH) region in which the full set of VH CDRs 1, 2, and 3 (combined) is identical to the VH CDRs 1, 2, and 3 of any one of SEQ ID NOs: 158-314. In some embodiments, the antigen-binding protein (e.g., the anti-GIPR antibody) comprises a heavy chain variable (VH) region in which the full set of VH CDRs 1, 2, and 3 (combined) is identical to the VH CDRs 1, 2, and 3 of any one of SEQ ID NOs: 158-314, wherein the VH CDRs 1, 2, and 3 are defined by any one of Kabat, Chothia, or IGMT. In some embodiments, the antigen-binding protein (e.g., the anti-GIPR antibody) comprises a heavy chain variable (VH) region in which the full set of VH CDRs 1, 2, and 3 (combined) is identical to the VH CDRs 1, 2, and 3 of any one of SEQ ID NOs: 158-314, wherein the VH CDRs 1, 2, and 3 are defined by Kabat. In some embodiments, the antigen-binding protein (e.g., the anti-GIPR antibody) comprises a heavy chain variable (VH) region in which the full set of VH CDRs 1, 2, and 3 (combined) is identical to the VH CDRs 1, 2, and 3 of any one of SEQ ID NOs: 158-314, wherein the VH CDRs 1, 2, and 3 are defined by Chothia. In some embodiments, the antigen-binding protein (e.g., the anti-GIPR antibody) comprises a heavy chain variable (VH) region in which the full set of VH CDRs 1, 2, and 3 (combined) is identical to the VH CDRs 1, 2, and 3 of any one of SEQ ID NOs: 158-314, wherein the VH CDRs 1, 2, and 3 are defined by IGMT.

In some embodiments, the antigen-binding protein (e.g., the anti-GIPR antibody) comprises a light chain variable (VL) region in which the full set of VL CDRs 1, 2, and 3 (combined) is identical to the VL CDRs 1, 2, and 3 of an antibody of Table 9 and a heavy chain variable (VH) region in which the full set of VH CDRs 1, 2, and 3 (combined) is identical to the VH CDRs 1, 2, and 3 of an antibody of Table 9. In some embodiments, the antigen-binding protein (e.g., the anti-GIPR antibody) comprises a VL region in which the full set of VL CDRs 1, 2, and 3 (combined) is identical to the VL CDRs 1, 2, and 3 of an antibody of Table 9 and a VH region in which the full set of VH CDRs 1, 2, and 3 (combined) is identical to the VH CDRs 1, 2, and 3 of an antibody of Table 9, wherein the VL CDRs 1, 2, and 3 and the VH CDRs 1, 2, and 3 are defined by any one of Kabat, Chothia, or IGMT. In some embodiments, the VL CDRs 1, 2, and 3 and the VH CDRs 1, 2, and 3 are defined by Kabat. In some embodiments, the VL CDRs 1, 2, and 3 and the VH CDRs 1, 2, and 3 are defined by Chothia. In some embodiments, the VL CDRs 1, 2, and 3 and the VH CDRs 1, 2, and 3 are defined by IGMT.

In some embodiments, the antigen-binding protein (e.g., the anti-GIPR antibody) comprises a light chain variable (VL) region in which the full set of VL CDRs 1, 2, and 3 (combined) is identical to the VL CDRs 1, 2, and 3 of SEQ ID NO: 74 and a heavy chain variable (VH) region in which the full set of VH CDRs 1, 2, and 3 (combined) is identical to the VH CDRs 1, 2, and 3 of SEQ ID NO: 231. In some embodiments, the antigen-binding protein (e.g., the anti-GIPR antibody) comprises a VL region in which the full set of VL CDRs 1, 2, and 3 (combined) is identical to the VL CDRs 1, 2, and 3 of SEQ ID NO: 74 and a VH region in which the full set of VH CDRs 1, 2, and 3 (combined) is identical to the VH CDRs 1, 2, and 3 of SEQ ID NO: 231, wherein the VL CDRs 1, 2, and 3 and the VH CDRs 1, 2, and 3 are defined by any one of Kabat, Chothia, or IGMT. In some embodiments, the VL CDRs 1, 2, and 3 and the VH CDRs 1, 2, and 3 are defined by Kabat. In some embodiments, the VL CDRs 1, 2, and 3 and the VH CDRs 1, 2, and 3 are defined by Chothia. In some embodiments, the VL CDRs 1, 2, and 3 and the VH CDRs 1, 2, and 3 are defined by IGMT.

In some embodiments, the antigen-binding protein (e.g., the anti-GIPR antibody) comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise amino acid sequences selected from:
i. SEQ ID NO: 629, SEQ ID NO: 786, SEQ ID NO: 943, SEQ ID NO: 1100, SEQ ID NO: 1257, and SEQ ID NO: 1414, respectively;
ii. SEQ ID NO: 630, SEQ ID NO: 787, SEQ ID NO: 944, SEQ ID NO: 1101, SEQ ID NO: 1258, and SEQ ID NO: 1415, respectively;
iii. SEQ ID NO: 631, SEQ ID NO: 788, SEQ ID NO: 945, SEQ ID NO: 1102, SEQ ID NO: 1259, and SEQ ID NO: 1416, respectively;
iv. SEQ ID NO: 632, SEQ ID NO: 789, SEQ ID NO: 946, SEQ ID NO: 1103, SEQ ID NO: 1260, and SEQ ID NO: 1417, respectively;
v. SEQ ID NO: 633, SEQ ID NO: 790, SEQ ID NO: 947, SEQ ID NO: 1104, SEQ ID NO: 1261, and SEQ ID NO: 1418, respectively;
vi. SEQ ID NO: 634, SEQ ID NO: 791, SEQ ID NO: 948, SEQ ID NO: 1105, SEQ ID NO: 1262, and SEQ ID NO: 1419, respectively;
vii. SEQ ID NO: 635, SEQ ID NO: 792, SEQ ID NO: 949, SEQ ID NO: 1106, SEQ ID NO: 1263, and SEQ ID NO: 1420, respectively;
viii. SEQ ID NO: 636, SEQ ID NO: 793, SEQ ID NO: 950, SEQ ID NO: 1107, SEQ ID NO: 1264, and SEQ ID NO: 1421, respectively;
ix. SEQ ID NO: 637, SEQ ID NO: 794, SEQ ID NO: 951, SEQ ID NO: 1108, SEQ ID NO: 1265, and SEQ ID NO: 1422, respectively;
x. SEQ ID NO: 638, SEQ ID NO: 795, SEQ ID NO: 952, SEQ ID NO: 1109, SEQ ID NO: 1266, and SEQ ID NO: 1423, respectively;
xi. SEQ ID NO: 639, SEQ ID NO: 796, SEQ ID NO: 953, SEQ ID NO: 1110, SEQ ID NO: 1267, and SEQ ID NO: 1424, respectively;
xii. SEQ ID NO: 640, SEQ ID NO: 797, SEQ ID NO: 954, SEQ ID NO: 1111, SEQ ID NO: 1268, and SEQ ID NO: 1425, respectively;
xiii. SEQ ID NO: 641, SEQ ID NO: 798, SEQ ID NO: 955, SEQ ID NO: 1112, SEQ ID NO: 1269, and SEQ ID NO: 1426, respectively;
xiv. SEQ ID NO: 642, SEQ ID NO: 799, SEQ ID NO: 956, SEQ ID NO: 1113, SEQ ID NO: 1270, and SEQ ID NO: 1427, respectively;
xv. SEQ ID NO: 643, SEQ ID NO: 800, SEQ ID NO: 957, SEQ ID NO: 1114, SEQ ID NO: 1271, and SEQ ID NO: 1428, respectively;
xvi. SEQ ID NO: 644, SEQ ID NO: 801, SEQ ID NO: 958, SEQ ID NO: 1115, SEQ ID NO: 1272, and SEQ ID NO: 1429, respectively;
xvii. SEQ ID NO: 645, SEQ ID NO: 802, SEQ ID NO: 959, SEQ ID NO: 1116, SEQ ID NO: 1273, and SEQ ID NO: 1430, respectively;
xviii. SEQ ID NO: 646, SEQ ID NO: 803, SEQ ID NO: 960, SEQ ID NO: 1117, SEQ ID NO: 1274, and SEQ ID NO: 1431, respectively;
xix. SEQ ID NO: 647, SEQ ID NO: 804, SEQ ID NO: 961, SEQ ID NO: 1118, SEQ ID NO: 1275, and SEQ ID NO: 1432, respectively;
xx. SEQ ID NO: 648, SEQ ID NO: 805, SEQ ID NO: 962, SEQ ID NO: 1119, SEQ ID NO: 1276, and SEQ ID NO: 1433, respectively;
xxi. SEQ ID NO: 649, SEQ ID NO: 806, SEQ ID NO: 963, SEQ ID NO: 1120, SEQ ID NO: 1277, and SEQ ID NO: 1434, respectively;
xxii. SEQ ID NO: 650, SEQ ID NO: 807, SEQ ID NO: 964, SEQ ID NO: 1121, SEQ ID NO: 1278, and SEQ ID NO: 1435, respectively;
xxiii. SEQ ID NO: 651, SEQ ID NO: 808, SEQ ID NO: 965, SEQ ID NO: 1122, SEQ ID NO: 1279, and SEQ ID NO: 1436, respectively;
xxiv. SEQ ID NO: 652, SEQ ID NO: 809, SEQ ID NO: 966, SEQ ID NO: 1123, SEQ ID NO: 1280, and SEQ ID NO: 1437, respectively;
xxv. SEQ ID NO: 653, SEQ ID NO: 810, SEQ ID NO: 967, SEQ ID NO: 1124, SEQ ID NO: 1281, and SEQ ID NO: 1438, respectively;
xxvi. SEQ ID NO: 654, SEQ ID NO: 811, SEQ ID NO: 968, SEQ ID NO: 1125, SEQ ID NO: 1282, and SEQ ID NO: 1439, respectively;
xxvii. SEQ ID NO: 655, SEQ ID NO: 812, SEQ ID NO: 969, SEQ ID NO: 1126, SEQ ID NO: 1283, and SEQ ID NO: 1440, respectively;
xxviii. SEQ ID NO: 656, SEQ ID NO: 813, SEQ ID NO: 970, SEQ ID NO: 1127, SEQ ID NO: 1284, and SEQ ID NO: 1441, respectively;
xxix. SEQ ID NO: 657, SEQ ID NO: 814, SEQ ID NO: 971, SEQ ID NO: 1128, SEQ ID NO: 1285, and SEQ ID NO: 1442, respectively;
xxx. SEQ ID NO: 658, SEQ ID NO: 815, SEQ ID NO: 972, SEQ ID NO: 1129, SEQ ID NO: 1286, and SEQ ID NO: 1443, respectively;
xxxi. SEQ ID NO: 659, SEQ ID NO: 816, SEQ ID NO: 973, SEQ ID NO: 1130, SEQ ID NO: 1287, and SEQ ID NO: 1444, respectively;
xxxii. SEQ ID NO: 660, SEQ ID NO: 817, SEQ ID NO: 974, SEQ ID NO: 1131, SEQ ID NO: 1288, and SEQ ID NO: 1445, respectively;
xxxiii. SEQ ID NO: 661, SEQ ID NO: 818, SEQ ID NO: 975, SEQ ID NO: 1132, SEQ ID NO: 1289, and SEQ ID NO: 1446, respectively;
xxxiv. SEQ ID NO: 662, SEQ ID NO: 819, SEQ ID NO: 976, SEQ ID NO: 1133, SEQ ID NO: 1290, and SEQ ID NO: 1447, respectively;
xxxv. SEQ ID NO: 663, SEQ ID NO: 820, SEQ ID NO: 977, SEQ ID NO: 1134, SEQ ID NO: 1291, and SEQ ID NO: 1448, respectively;
xxxvi. SEQ ID NO: 664, SEQ ID NO: 821, SEQ ID NO: 978, SEQ ID NO: 1135, SEQ ID NO: 1292, and SEQ ID NO: 1449, respectively;
xxxvii. SEQ ID NO: 665, SEQ ID NO: 822, SEQ ID NO: 979, SEQ ID NO: 1136, SEQ ID NO: 1293, and SEQ ID NO: 1450, respectively;
xxxviii. SEQ ID NO: 666, SEQ ID NO: 823, SEQ ID NO: 980, SEQ ID NO: 1137, SEQ ID NO: 1294, and SEQ ID NO: 1451, respectively;
xxxix. SEQ ID NO: 667, SEQ ID NO: 824, SEQ ID NO: 981, SEQ ID NO: 1138, SEQ ID NO: 1295, and SEQ ID NO: 1452, respectively;
xl. SEQ ID NO: 668, SEQ ID NO: 825, SEQ ID NO: 982, SEQ ID NO: 1139, SEQ ID NO: 1296, and SEQ ID NO: 1453, respectively;
xli. SEQ ID NO: 669, SEQ ID NO: 826, SEQ ID NO: 983, SEQ ID NO: 1140, SEQ ID NO: 1297, and SEQ ID NO: 1454, respectively;
xlii. SEQ ID NO: 670, SEQ ID NO: 827, SEQ ID NO: 984, SEQ ID NO: 1141, SEQ ID NO: 1298, and SEQ ID NO: 1455, respectively;
xliii. SEQ ID NO: 671, SEQ ID NO: 828, SEQ ID NO: 985, SEQ ID NO: 1142, SEQ ID NO: 1299, and SEQ ID NO: 1456, respectively;
xliv. SEQ ID NO: 672, SEQ ID NO: 829, SEQ ID NO: 986, SEQ ID NO: 1143, SEQ ID NO: 1300, and SEQ ID NO: 1457, respectively;
xlv. SEQ ID NO: 673, SEQ ID NO: 830, SEQ ID NO: 987, SEQ ID NO: 1144, SEQ ID NO: 1301, and SEQ ID NO: 1458, respectively;
xlvi. SEQ ID NO: 674, SEQ ID NO: 831, SEQ ID NO: 988, SEQ ID NO: 1145, SEQ ID NO: 1302, and SEQ ID NO: 1459, respectively;
xlvii. SEQ ID NO: 675, SEQ ID NO: 832, SEQ ID NO: 989, SEQ ID NO: 1146, SEQ ID NO: 1303, and SEQ ID NO: 1460, respectively;
xlviii. SEQ ID NO: 676, SEQ ID NO: 833, SEQ ID NO: 990, SEQ ID NO: 1147, SEQ ID NO: 1304, and SEQ ID NO: 1461, respectively;
xlix. SEQ ID NO: 677, SEQ ID NO: 834, SEQ ID NO: 991, SEQ ID NO: 1148, SEQ ID NO: 1305, and SEQ ID NO: 1462, respectively;
l. SEQ ID NO: 678, SEQ ID NO: 835, SEQ ID NO: 992, SEQ ID NO: 1149, SEQ ID NO: 1306, and SEQ ID NO: 1463, respectively;
li. SEQ ID NO: 679, SEQ ID NO: 836, SEQ ID NO: 993, SEQ ID NO: 1150, SEQ ID NO: 1307, and SEQ ID NO: 1464, respectively;
lii. SEQ ID NO: 680, SEQ ID NO: 837, SEQ ID NO: 994, SEQ ID NO: 1151, SEQ ID NO: 1308, and SEQ ID NO: 1465, respectively;
liii. SEQ ID NO: 681, SEQ ID NO: 838, SEQ ID NO: 995, SEQ ID NO: 1152, SEQ ID NO: 1309, and SEQ ID NO: 1466, respectively;
liv. SEQ ID NO: 682, SEQ ID NO: 839, SEQ ID NO: 996, SEQ ID NO: 1153, SEQ ID NO: 1310, and SEQ ID NO: 1467, respectively;
lv. SEQ ID NO: 683, SEQ ID NO: 840, SEQ ID NO: 997, SEQ ID NO: 1154, SEQ ID NO: 1311, and SEQ ID NO: 1468, respectively;
lvi. SEQ ID NO: 684, SEQ ID NO: 841, SEQ ID NO: 998, SEQ ID NO: 1155, SEQ ID NO: 1312, and SEQ ID NO: 1469, respectively;
lvii. SEQ ID NO: 685, SEQ ID NO: 842, SEQ ID NO: 999, SEQ ID NO: 1156, SEQ ID NO: 1313, and SEQ ID NO: 1470, respectively;
lviii. SEQ ID NO: 686, SEQ ID NO: 843, SEQ ID NO: 1000, SEQ ID NO: 1157, SEQ ID NO: 1314, and SEQ ID NO: 1471, respectively;
lix. SEQ ID NO: 687, SEQ ID NO: 844, SEQ ID NO: 1001, SEQ ID NO: 1158, SEQ ID NO: 1315, and SEQ ID NO: 1472, respectively;
lx. SEQ ID NO: 688, SEQ ID NO: 845, SEQ ID NO: 1002, SEQ ID NO: 1159, SEQ ID NO: 1316, and SEQ ID NO: 1473, respectively;
lxi. SEQ ID NO: 689, SEQ ID NO: 846, SEQ ID NO: 1003, SEQ ID NO: 1160, SEQ ID NO: 1317, and SEQ ID NO: 1474, respectively;
lxii. SEQ ID NO: 690, SEQ ID NO: 847, SEQ ID NO: 1004, SEQ ID NO: 1161, SEQ ID NO: 1318, and SEQ ID NO: 1475, respectively;
lxiii. SEQ ID NO: 691, SEQ ID NO: 848, SEQ ID NO: 1005, SEQ ID NO: 1162, SEQ ID NO: 1319, and SEQ ID NO: 1476, respectively;
lxiv. SEQ ID NO: 692, SEQ ID NO: 849, SEQ ID NO: 1006, SEQ ID NO: 1163, SEQ ID NO: 1320, and SEQ ID NO: 1477, respectively;
lxv. SEQ ID NO: 693, SEQ ID NO: 850, SEQ ID NO: 1007, SEQ ID NO: 1164, SEQ ID NO: 1321, and SEQ ID NO: 1478, respectively;
lxvi. SEQ ID NO: 694, SEQ ID NO: 851, SEQ ID NO: 1008, SEQ ID NO: 1165, SEQ ID NO: 1322, and SEQ ID NO: 1479, respectively;
lxvii. SEQ ID NO: 695, SEQ ID NO: 852, SEQ ID NO: 1009, SEQ ID NO: 1166, SEQ ID NO: 1323, and SEQ ID NO: 1480, respectively;
lxviii. SEQ ID NO: 696, SEQ ID NO: 853, SEQ ID NO: 1010, SEQ ID NO: 1167, SEQ ID NO: 1324, and SEQ ID NO: 1481, respectively;
lxix. SEQ ID NO: 697, SEQ ID NO: 854, SEQ ID NO: 1011, SEQ ID NO: 1168, SEQ ID NO: 1325, and SEQ ID NO: 1482, respectively;
lxx. SEQ ID NO: 698, SEQ ID NO: 855, SEQ ID NO: 1012, SEQ ID NO: 1169, SEQ ID NO: 1326, and SEQ ID NO: 1483, respectively;
lxxi. SEQ ID NO: 699, SEQ ID NO: 856, SEQ ID NO: 1013, SEQ ID NO: 1170, SEQ ID NO: 1327, and SEQ ID NO: 1484, respectively;
lxxii. SEQ ID NO: 700, SEQ ID NO: 857, SEQ ID NO: 1014, SEQ ID NO: 1171, SEQ ID NO: 1328, and SEQ ID NO: 1485, respectively;
lxxiii. SEQ ID NO: 701, SEQ ID NO: 858, SEQ ID NO: 1015, SEQ ID NO: 1172, SEQ ID NO: 1329, and SEQ ID NO: 1486, respectively;
lxxiv. SEQ ID NO: 702, SEQ ID NO: 859, SEQ ID NO: 1016, SEQ ID NO: 1173, SEQ ID NO: 1330, and SEQ ID NO: 1487, respectively;
lxxv. SEQ ID NO: 703, SEQ ID NO: 860, SEQ ID NO: 1017, SEQ ID NO: 1174, SEQ ID NO: 1331, and SEQ ID NO: 1488, respectively;
lxxvi. SEQ ID NO: 704, SEQ ID NO: 861, SEQ ID NO: 1018, SEQ ID NO: 1175, SEQ ID NO: 1332, and SEQ ID NO: 1489, respectively;
lxxvii. SEQ ID NO: 705, SEQ ID NO: 862, SEQ ID NO: 1019, SEQ ID NO: 1176, SEQ ID NO: 1333, and SEQ ID NO: 1490, respectively;
lxxviii. SEQ ID NO: 706, SEQ ID NO: 863, SEQ ID NO: 1020, SEQ ID NO: 1177, SEQ ID NO: 1334, and SEQ ID NO: 1491, respectively;
lxxix. SEQ ID NO: 707, SEQ ID NO: 864, SEQ ID NO: 1021, SEQ ID NO: 1178, SEQ ID NO: 1335, and SEQ ID NO: 1492, respectively;
lxxx. SEQ ID NO: 708, SEQ ID NO: 865, SEQ ID NO: 1022, SEQ ID NO: 1179, SEQ ID NO: 1336, and SEQ ID NO: 1493, respectively;
lxxxi. SEQ ID NO: 709, SEQ ID NO: 866, SEQ ID NO: 1023, SEQ ID NO: 1180, SEQ ID NO: 1337, and SEQ ID NO: 1494, respectively;
lxxxii. SEQ ID NO: 710, SEQ ID NO: 867, SEQ ID NO: 1024, SEQ ID NO: 1181, SEQ ID NO: 1338, and SEQ ID NO: 1495, respectively;
lxxxiii. SEQ ID NO: 711, SEQ ID NO: 868, SEQ ID NO: 1025, SEQ ID NO: 1182, SEQ ID NO: 1339, and SEQ ID NO: 1496, respectively;
lxxxiv. SEQ ID NO: 712, SEQ ID NO: 869, SEQ ID NO: 1026, SEQ ID NO: 1183, SEQ ID NO: 1340, and SEQ ID NO: 1497, respectively;
lxxxv. SEQ ID NO: 713, SEQ ID NO: 870, SEQ ID NO: 1027, SEQ ID NO: 1184, SEQ ID NO: 1341, and SEQ ID NO: 1498, respectively;
lxxxvi. SEQ ID NO: 714, SEQ ID NO: 871, SEQ ID NO: 1028, SEQ ID NO: 1185, SEQ ID NO: 1342, and SEQ ID NO: 1499, respectively;
lxxxvii. SEQ ID NO: 715, SEQ ID NO: 872, SEQ ID NO: 1029, SEQ ID NO: 1186, SEQ ID NO: 1343, and SEQ ID NO: 1500, respectively;
lxxxviii. SEQ ID NO: 716, SEQ ID NO: 873, SEQ ID NO: 1030, SEQ ID NO: 1187, SEQ ID NO: 1344, and SEQ ID NO: 1501, respectively;
lxxxix. SEQ ID NO: 717, SEQ ID NO: 874, SEQ ID NO: 1031, SEQ ID NO: 1188, SEQ ID NO: 1345, and SEQ ID NO: 1502, respectively;
xc. SEQ ID NO: 718, SEQ ID NO: 875, SEQ ID NO: 1032, SEQ ID NO: 1189, SEQ ID NO: 1346, and SEQ ID NO: 1503, respectively;
xci. SEQ ID NO: 719, SEQ ID NO: 876, SEQ ID NO: 1033, SEQ ID NO: 1190, SEQ ID NO: 1347, and SEQ ID NO: 1504, respectively;
xcii. SEQ ID NO: 720, SEQ ID NO: 877, SEQ ID NO: 1034, SEQ ID NO: 1191, SEQ ID NO: 1348, and SEQ ID NO: 1505, respectively;
xciii. SEQ ID NO: 721, SEQ ID NO: 878, SEQ ID NO: 1035, SEQ ID NO: 1192, SEQ ID NO: 1349, and SEQ ID NO: 1506, respectively;
xciv. SEQ ID NO: 722, SEQ ID NO: 879, SEQ ID NO: 1036, SEQ ID NO: 1193, SEQ ID NO: 1350, and SEQ ID NO: 1507, respectively;
xcv. SEQ ID NO: 723, SEQ ID NO: 880, SEQ ID NO: 1037, SEQ ID NO: 1194, SEQ ID NO: 1351, and SEQ ID NO: 1508, respectively;
xcvi. SEQ ID NO: 724, SEQ ID NO: 881, SEQ ID NO: 1038, SEQ ID NO: 1195, SEQ ID NO: 1352, and SEQ ID NO: 1509, respectively;
xcvii. SEQ ID NO: 725, SEQ ID NO: 882, SEQ ID NO: 1039, SEQ ID NO: 1196, SEQ ID NO: 1353, and SEQ ID NO: 1510, respectively;
xcviii. SEQ ID NO: 726, SEQ ID NO: 883, SEQ ID NO: 1040, SEQ ID NO: 1197, SEQ ID NO: 1354, and SEQ ID NO: 1511, respectively;
xcix. SEQ ID NO: 727, SEQ ID NO: 884, SEQ ID NO: 1041, SEQ ID NO: 1198, SEQ ID NO: 1355, and SEQ ID NO: 1512, respectively;
c. SEQ ID NO: 728, SEQ ID NO: 885, SEQ ID NO: 1042, SEQ ID NO: 1199, SEQ ID NO: 1356, and SEQ ID NO: 1513, respectively;
ci. SEQ ID NO: 729, SEQ ID NO: 886, SEQ ID NO: 1043, SEQ ID NO: 1200, SEQ ID NO: 1357, and SEQ ID NO: 1514, respectively;
cii. SEQ ID NO: 730, SEQ ID NO: 887, SEQ ID NO: 1044, SEQ ID NO: 1201, SEQ ID NO: 1358, and SEQ ID NO: 1515, respectively;
ciii. SEQ ID NO: 731, SEQ ID NO: 888, SEQ ID NO: 1045, SEQ ID NO: 1202, SEQ ID NO: 1359, and SEQ ID NO: 1516, respectively;
civ. SEQ ID NO: 732, SEQ ID NO: 889, SEQ ID NO: 1046, SEQ ID NO: 1203, SEQ ID NO: 1360, and SEQ ID NO: 1517, respectively;
cv. SEQ ID NO: 733, SEQ ID NO: 890, SEQ ID NO: 1047, SEQ ID NO: 1204, SEQ ID NO: 1361, and SEQ ID NO: 1518, respectively;
cvi. SEQ ID NO: 734, SEQ ID NO: 891, SEQ ID NO: 1048, SEQ ID NO: 1205, SEQ ID NO: 1362, and SEQ ID NO: 1519, respectively;
cvii. SEQ ID NO: 735, SEQ ID NO: 892, SEQ ID NO: 1049, SEQ ID NO: 1206, SEQ ID NO: 1363, and SEQ ID NO: 1520, respectively;
cviii. SEQ ID NO: 736, SEQ ID NO: 893, SEQ ID NO: 1050, SEQ ID NO: 1207, SEQ ID NO: 1364, and SEQ ID NO: 1521, respectively;
cix. SEQ ID NO: 737, SEQ ID NO: 894, SEQ ID NO: 1051, SEQ ID NO: 1208, SEQ ID NO: 1365, and SEQ ID NO: 1522, respectively;
cx. SEQ ID NO: 738, SEQ ID NO: 895, SEQ ID NO: 1052, SEQ ID NO: 1209, SEQ ID NO: 1366, and SEQ ID NO: 1523, respectively;
cxi. SEQ ID NO: 739, SEQ ID NO: 896, SEQ ID NO: 1053, SEQ ID NO: 1210, SEQ ID NO: 1367, and SEQ ID NO: 1524, respectively;
cxii. SEQ ID NO: 740, SEQ ID NO: 897, SEQ ID NO: 1054, SEQ ID NO: 1211, SEQ ID NO: 1368, and SEQ ID NO: 1525, respectively;
cxiii. SEQ ID NO: 741, SEQ ID NO: 898, SEQ ID NO: 1055, SEQ ID NO: 1212, SEQ ID NO: 1369, and SEQ ID NO: 1526, respectively;
cxiv. SEQ ID NO: 742, SEQ ID NO: 899, SEQ ID NO: 1056, SEQ ID NO: 1213, SEQ ID NO: 1370, and SEQ ID NO: 1527, respectively;
cxv. SEQ ID NO: 743, SEQ ID NO: 900, SEQ ID NO: 1057, SEQ ID NO: 1214, SEQ ID NO: 1371, and SEQ ID NO: 1528, respectively;
cxvi. SEQ ID NO: 744, SEQ ID NO: 901, SEQ ID NO: 1058, SEQ ID NO: 1215, SEQ ID NO: 1372, and SEQ ID NO: 1529, respectively;
cxvii. SEQ ID NO: 745, SEQ ID NO: 902, SEQ ID NO: 1059, SEQ ID NO: 1216, SEQ ID NO: 1373, and SEQ ID NO: 1530, respectively;
cxviii. SEQ ID NO: 746, SEQ ID NO: 903, SEQ ID NO: 1060, SEQ ID NO: 1217, SEQ ID NO: 1374, and SEQ ID NO: 1531, respectively;
cxix. SEQ ID NO: 747, SEQ ID NO: 904, SEQ ID NO: 1061, SEQ ID NO: 1218, SEQ ID NO: 1375, and SEQ ID NO: 1532, respectively;
cxx. SEQ ID NO: 748, SEQ ID NO: 905, SEQ ID NO: 1062, SEQ ID NO: 1219, SEQ ID NO: 1376, and SEQ ID NO: 1533, respectively;
cxxi. SEQ ID NO: 749, SEQ ID NO: 906, SEQ ID NO: 1063, SEQ ID NO: 1220, SEQ ID NO: 1377, and SEQ ID NO: 1534, respectively;
cxxii. SEQ ID NO: 750, SEQ ID NO: 907, SEQ ID NO: 1064, SEQ ID NO: 1221, SEQ ID NO: 1378, and SEQ ID NO: 1535, respectively;
cxxiii. SEQ ID NO: 751, SEQ ID NO: 908, SEQ ID NO: 1065, SEQ ID NO: 1222, SEQ ID NO: 1379, and SEQ ID NO: 1536, respectively;
cxxiv. SEQ ID NO: 752, SEQ ID NO: 909, SEQ ID NO: 1066, SEQ ID NO: 1223, SEQ ID NO: 1380, and SEQ ID NO: 1537, respectively;
cxxv. SEQ ID NO: 753, SEQ ID NO: 910, SEQ ID NO: 1067, SEQ ID NO: 1224, SEQ ID NO: 1381, and SEQ ID NO: 1538, respectively;
cxxvi. SEQ ID NO: 754, SEQ ID NO: 911, SEQ ID NO: 1068, SEQ ID NO: 1225, SEQ ID NO: 1382, and SEQ ID NO: 1539, respectively;
cxxvii. SEQ ID NO: 755, SEQ ID NO: 912, SEQ ID NO: 1069, SEQ ID NO: 1226, SEQ ID NO: 1383, and SEQ ID NO: 1540, respectively;
cxxviii. SEQ ID NO: 756, SEQ ID NO: 913, SEQ ID NO: 1070, SEQ ID NO: 1227, SEQ ID NO: 1384, and SEQ ID NO: 1541, respectively;
cxxix. SEQ ID NO: 757, SEQ ID NO: 914, SEQ ID NO: 1071, SEQ ID NO: 1228, SEQ ID NO: 1385, and SEQ ID NO: 1542, respectively;
cxxx. SEQ ID NO: 758, SEQ ID NO: 915, SEQ ID NO: 1072, SEQ ID NO: 1229, SEQ ID NO: 1386, and SEQ ID NO: 1543, respectively;
cxxxi. SEQ ID NO: 759, SEQ ID NO: 916, SEQ ID NO: 1073, SEQ ID NO: 1230, SEQ ID NO: 1387, and SEQ ID NO: 1544, respectively;
cxxxii. SEQ ID NO: 760, SEQ ID NO: 917, SEQ ID NO: 1074, SEQ ID NO: 1231, SEQ ID NO: 1388, and SEQ ID NO: 1545, respectively;
cxxxiii. SEQ ID NO: 761, SEQ ID NO: 918, SEQ ID NO: 1075, SEQ ID NO: 1232, SEQ ID NO: 1389, and SEQ ID NO: 1546, respectively;
cxxxiv. SEQ ID NO: 762, SEQ ID NO: 919, SEQ ID NO: 1076, SEQ ID NO: 1233, SEQ ID NO: 1390, and SEQ ID NO: 1547, respectively;
cxxxv. SEQ ID NO: 763, SEQ ID NO: 920, SEQ ID NO: 1077, SEQ ID NO: 1234, SEQ ID NO: 1391, and SEQ ID NO: 1548, respectively;
cxxxvi. SEQ ID NO: 764, SEQ ID NO: 921, SEQ ID NO: 1078, SEQ ID NO: 1235, SEQ ID NO: 1392, and SEQ ID NO: 1549, respectively;
cxxxvii. SEQ ID NO: 765, SEQ ID NO: 922, SEQ ID NO: 1079, SEQ ID NO: 1236, SEQ ID NO: 1393, and SEQ ID NO: 1550, respectively;
cxxxviii. SEQ ID NO: 766, SEQ ID NO: 923, SEQ ID NO: 1080, SEQ ID NO: 1237, SEQ ID NO: 1394, and SEQ ID NO: 1551, respectively;
cxxxix. SEQ ID NO: 767, SEQ ID NO: 924, SEQ ID NO: 1081, SEQ ID NO: 1238, SEQ ID NO: 1395, and SEQ ID NO: 1552, respectively;
cxl. SEQ ID NO: 768, SEQ ID NO: 925, SEQ ID NO: 1082, SEQ ID NO: 1239, SEQ ID NO: 1396, and SEQ ID NO: 1553, respectively;
cxli. SEQ ID NO: 769, SEQ ID NO: 926, SEQ ID NO: 1083, SEQ ID NO: 1240, SEQ ID NO: 1397, and SEQ ID NO: 1554, respectively;
cxlii. SEQ ID NO: 770, SEQ ID NO: 927, SEQ ID NO: 1084, SEQ ID NO: 1241, SEQ ID NO: 1398, and SEQ ID NO: 1555, respectively;
cxliii. SEQ ID NO: 771, SEQ ID NO: 928, SEQ ID NO: 1085, SEQ ID NO: 1242, SEQ ID NO: 1399, and SEQ ID NO: 1556, respectively;
cxliv. SEQ ID NO: 772, SEQ ID NO: 929, SEQ ID NO: 1086, SEQ ID NO: 1243, SEQ ID NO: 1400, and SEQ ID NO: 1557, respectively;
cxlv. SEQ ID NO: 773, SEQ ID NO: 930, SEQ ID NO: 1087, SEQ ID NO: 1244, SEQ ID NO: 1401, and SEQ ID NO: 1558, respectively;
cxlvi. SEQ ID NO: 774, SEQ ID NO: 931, SEQ ID NO: 1088, SEQ ID NO: 1245, SEQ ID NO: 1402, and SEQ ID NO: 1559, respectively;
cxlvii. SEQ ID NO: 775, SEQ ID NO: 932, SEQ ID NO: 1089, SEQ ID NO: 1246, SEQ ID NO: 1403, and SEQ ID NO: 1560, respectively;
cxlviii. SEQ ID NO: 776, SEQ ID NO: 933, SEQ ID NO: 1090, SEQ ID NO: 1247, SEQ ID NO: 1404, and SEQ ID NO: 1561, respectively;
cxlix. SEQ ID NO: 777, SEQ ID NO: 934, SEQ ID NO: 1091, SEQ ID NO: 1248, SEQ ID NO: 1405, and SEQ ID NO: 1562, respectively;
cl. SEQ ID NO: 778, SEQ ID NO: 935, SEQ ID NO: 1092, SEQ ID NO: 1249, SEQ ID NO: 1406, and SEQ ID NO: 1563, respectively;
cli. SEQ ID NO: 779, SEQ ID NO: 936, SEQ ID NO: 1093, SEQ ID NO: 1250, SEQ ID NO: 1407, and SEQ ID NO: 1564, respectively;
clii. SEQ ID NO: 780, SEQ ID NO: 937, SEQ ID NO: 1094, SEQ ID NO: 1251, SEQ ID NO: 1408, and SEQ ID NO: 1565, respectively;
cliii. SEQ ID NO: 781, SEQ ID NO: 938, SEQ ID NO: 1095, SEQ ID NO: 1252, SEQ ID NO: 1409, and SEQ ID NO: 1566, respectively;
cliv. SEQ ID NO: 782, SEQ ID NO: 939, SEQ ID NO: 1096, SEQ ID NO: 1253, SEQ ID NO: 1410, and SEQ ID NO: 1567, respectively;
clv. SEQ ID NO: 783, SEQ ID NO: 940, SEQ ID NO: 1097, SEQ ID NO: 1254, SEQ ID NO: 1411, and SEQ ID NO: 1568, respectively;
clvi. SEQ ID NO: 784, SEQ ID NO: 941, SEQ ID NO: 1098, SEQ ID NO: 1255, SEQ ID NO: 1412, and SEQ ID NO: 1569, respectively; and
clvii. SEQ ID NO: 785, SEQ ID NO: 942, SEQ ID NO: 1099, SEQ ID NO: 1256, SEQ ID NO: 1413, and SEQ ID NO: 1570, respectively,
preferably wherein the CDRL1, the CDRL2, the CDRL3, the CDRH1, the CDRH2, and the CDRH3 comprise the amino acid sequences of SEQ ID NO: 702, SEQ ID NO: 859, SEQ ID NO: 1016, SEQ ID NO: 1173, SEQ ID NO: 1330, and SEQ ID NO: 1487, respectively.

In some embodiments, the antigen-binding protein is an antibody comprising at least one cysteine conjugation site at a position selected from the group consisting of 88 (e.g., D88) of the light chain, 384 (e.g., E384) of the heavy chain, and 487 (e.g., T487) of the heavy chain, all according to AHo numbering.

In some embodiments, the antigen-binding protein (e.g., the anti-GIPR antibody) comprises a full-length light chain and a full-length heavy chain as listed in one of the rows for one of the antibodies listed in Tables 9, 14, and 15.

In some embodiments, the antigen-binding protein (e.g., the anti-GIPR antibody) comprises a full-length light chain and a full-length heavy chain as listed in one of the rows for one of the antibodies listed in Tables 9, 14, and 15, except that one or both of the chains differs from the amino acid sequence specified in the table at only 1, 2, 3, 4, 5, 6, 7, 8, 9, 10, 11, 12, 13, 14, or 15 amino acid residues, wherein each such sequence difference is independently a single amino acid deletion, insertion, or substitution, with the deletions, insertions, and/or substitutions resulting in no more than 1, 2, 3, 4, 5, 6, 7, 8, 9, 10, 11, 12, 13, 14 or 15 amino acid changes relative to the full-length sequences specified in Tables 14 and 15.

In some embodiments, the antigen-binding protein (e.g., the anti-GIPR antibody) comprises a full-length light chain and/or a full-length heavy chain from Table 14 or Table 15, respectively, with the N-terminal methionine deleted.

In some embodiments, the antigen-binding protein (e.g., the anti-GIPR antibody) comprises a full-length light chain and/or a full length heavy chain from Table 14 or Table 15, respectively with the C-terminal lysine deleted.

In some embodiments, the antigen-binding protein (e.g., the anti-GIPR antibody) comprises a full-length light chain and a full-length heavy chain as listed in one of the rows for one of the antibodies listed in Tables 9, 14, and 15, except that one or both of the chains differs from the amino acid sequence specified in Tables 14 and 15 in that the light chain and/or heavy chain comprises or consists of a sequence of amino acids that has at least 70%, at least 75%, at least 80%, at least 85%, at least 90%, at least 95%, at least 96%, at least 97%, at least 98%, or at least 99% sequence identity to the amino acid sequences of the light chain or heavy chain sequences as specified in Table 14 or Table 15, respectively.

In some embodiments, the antigen-binding protein (e.g., the anti-GIPR antibody) consists of a just a light or a heavy chain polypeptide as set forth in Table 14 or Table 15, respectively.

In some embodiments, the antigen-binding protein is an antibody comprising a light chain comprising an amino acid sequence selected from the group consisting of SEQ ID NOs: 472-628 and a heavy chain comprising an amino acid sequence selected from the group consisting of SEQ ID NOs: 472-628. In some embodiments, the antibody comprises at least one cysteine conjugation site at a position selected from the group consisting of 88 (e.g., D88) of the light chain, 384 (e.g., E384) of the heavy chain, and 487 (e.g., T487) of the heavy chain, all according to AHo numbering.

In some embodiments, the antigen-binding protein (e.g., the anti-GIPR antibody) comprises a light chain and a heavy chain, wherein the light chain and heavy chain comprise amino acid sequences selected from:
i. SEQ ID NO: 315 and SEQ ID NO: 472, respectively;
ii. SEQ ID NO: 316 and SEQ ID NO: 473, respectively;
iii. SEQ ID NO: 317 and SEQ ID NO: 474, respectively;
iv. SEQ ID NO: 318 and SEQ ID NO: 475, respectively;
v. SEQ ID NO: 319 and SEQ ID NO: 476, respectively;
vi. SEQ ID NO: 320 and SEQ ID NO: 477, respectively;
vii. SEQ ID NO: 321 and SEQ ID NO: 478, respectively;
viii. SEQ ID NO: 322 and SEQ ID NO: 479, respectively;
ix. SEQ ID NO: 323 and SEQ ID NO: 480, respectively;
x. SEQ ID NO: 324 and SEQ ID NO: 481, respectively;
xi. SEQ ID NO: 325 and SEQ ID NO: 482, respectively;
xii. SEQ ID NO: 326 and SEQ ID NO: 483, respectively;
xiii. SEQ ID NO: 327 and SEQ ID NO: 484, respectively;
xiv. SEQ ID NO: 328 and SEQ ID NO: 485, respectively;
xv. SEQ ID NO: 329 and SEQ ID NO: 486, respectively;
xvi. SEQ ID NO: 330 and SEQ ID NO: 487, respectively;
xvii. SEQ ID NO: 331 and SEQ ID NO: 488, respectively;
xviii. SEQ ID NO: 332 and SEQ ID NO: 489, respectively;
xix. SEQ ID NO: 333 and SEQ ID NO: 490, respectively;
xx. SEQ ID NO: 334 and SEQ ID NO: 491, respectively;
xxi. SEQ ID NO: 335 and SEQ ID NO: 492, respectively;
xxii. SEQ ID NO: 336 and SEQ ID NO: 493, respectively;
xxiii. SEQ ID NO: 337 and SEQ ID NO: 494, respectively;
xxiv. SEQ ID NO: 338 and SEQ ID NO: 495, respectively;
xxv. SEQ ID NO: 339 and SEQ ID NO: 496, respectively;
xxvi. SEQ ID NO: 340 and SEQ ID NO: 497, respectively;
xxvii. SEQ ID NO: 341 and SEQ ID NO: 498, respectively;
xxviii. SEQ ID NO: 342 and SEQ ID NO: 499, respectively;
xxix. SEQ ID NO: 343 and SEQ ID NO: 500, respectively;
xxx. SEQ ID NO: 344 and SEQ ID NO: 501, respectively;
xxxi. SEQ ID NO: 345 and SEQ ID NO: 502, respectively;
xxxii. SEQ ID NO: 346 and SEQ ID NO: 503, respectively;
xxxiii. SEQ ID NO: 347 and SEQ ID NO: 504, respectively;
xxxiv. SEQ ID NO: 348 and SEQ ID NO: 505, respectively;
xxxv. SEQ ID NO: 349 and SEQ ID NO: 506, respectively;
xxxvi. SEQ ID NO: 350 and SEQ ID NO: 507, respectively;
xxxvii. SEQ ID NO: 351 and SEQ ID NO: 508, respectively;
xxxviii. SEQ ID NO: 352 and SEQ ID NO: 509, respectively;
xxxix. SEQ ID NO: 353 and SEQ ID NO: 510, respectively;
xl. SEQ ID NO: 354 and SEQ ID NO: 511, respectively;
xli. SEQ ID NO: 355 and SEQ ID NO: 512, respectively;
xlii. SEQ ID NO: 356 and SEQ ID NO: 513, respectively;
xliii. SEQ ID NO: 357 and SEQ ID NO: 514, respectively;
xliv. SEQ ID NO: 358 and SEQ ID NO: 515, respectively;
xlv. SEQ ID NO: 359 and SEQ ID NO: 516, respectively;
xlvi. SEQ ID NO: 360 and SEQ ID NO: 517, respectively;
xlvii. SEQ ID NO: 361 and SEQ ID NO: 518, respectively;
xlviii. SEQ ID NO: 362 and SEQ ID NO: 519, respectively;
xlix. SEQ ID NO: 363 and SEQ ID NO: 520, respectively;
l. SEQ ID NO: 364 and SEQ ID NO: 521, respectively;
li. SEQ ID NO: 365 and SEQ ID NO: 522, respectively;
lii. SEQ ID NO: 366 and SEQ ID NO: 523, respectively;
liii. SEQ ID NO: 367 and SEQ ID NO: 524, respectively;
liv. SEQ ID NO: 368 and SEQ ID NO: 525, respectively;
lv. SEQ ID NO: 369 and SEQ ID NO: 526, respectively;
lvi. SEQ ID NO: 370 and SEQ ID NO: 527, respectively;
lvii. SEQ ID NO: 371 and SEQ ID NO: 528, respectively;
lviii. SEQ ID NO: 372 and SEQ ID NO: 529, respectively;
lix. SEQ ID NO: 373 and SEQ ID NO: 530, respectively;
lx. SEQ ID NO: 374 and SEQ ID NO: 531, respectively;
lxi. SEQ ID NO: 375 and SEQ ID NO: 532, respectively;
lxii. SEQ ID NO: 376 and SEQ ID NO: 533, respectively;
lxiii. SEQ ID NO: 377 and SEQ ID NO: 534, respectively;
lxiv. SEQ ID NO: 378 and SEQ ID NO: 535, respectively;
lxv. SEQ ID NO: 379 and SEQ ID NO: 536, respectively;
lxvi. SEQ ID NO: 380 and SEQ ID NO: 537, respectively;
lxvii. SEQ ID NO: 381 and SEQ ID NO: 538, respectively;
lxviii. SEQ ID NO: 382 and SEQ ID NO: 539, respectively;
lxix. SEQ ID NO: 383 and SEQ ID NO: 540, respectively;
lxx. SEQ ID NO: 384 and SEQ ID NO: 541, respectively;
lxxi. SEQ ID NO: 385 and SEQ ID NO: 542, respectively;
lxxii. SEQ ID NO: 386 and SEQ ID NO: 543, respectively;
lxxiii. SEQ ID NO: 387 and SEQ ID NO: 544, respectively;
lxxiv. SEQ ID NO: 388 and SEQ ID NO: 545, respectively;
lxxv. SEQ ID NO: 389 and SEQ ID NO: 546, respectively;
lxxvi. SEQ ID NO: 390 and SEQ ID NO: 547, respectively;
lxxvii. SEQ ID NO: 391 and SEQ ID NO: 548, respectively;
lxxviii. SEQ ID NO: 392 and SEQ ID NO: 549, respectively;
lxxix. SEQ ID NO: 393 and SEQ ID NO: 550, respectively;
lxxx. SEQ ID NO: 394 and SEQ ID NO: 551, respectively;
lxxxi. SEQ ID NO: 395 and SEQ ID NO: 552, respectively;
lxxxii. SEQ ID NO: 396 and SEQ ID NO: 553, respectively;
lxxxiii. SEQ ID NO: 397 and SEQ ID NO: 554, respectively;
lxxxiv. SEQ ID NO: 398 and SEQ ID NO: 555, respectively;
lxxxv. SEQ ID NO: 399 and SEQ ID NO: 556, respectively;
lxxxvi. SEQ ID NO: 400 and SEQ ID NO: 557, respectively;
lxxxvii. SEQ ID NO: 401 and SEQ ID NO: 558, respectively;
lxxxviii. SEQ ID NO: 402 and SEQ ID NO: 559, respectively;
lxxxix. SEQ ID NO: 403 and SEQ ID NO: 560, respectively;
xc. SEQ ID NO: 404 and SEQ ID NO: 561, respectively;
xci. SEQ ID NO: 405 and SEQ ID NO: 562, respectively;
xcii. SEQ ID NO: 406 and SEQ ID NO: 563, respectively;
xciii. SEQ ID NO: 407 and SEQ ID NO: 564, respectively;
xciv. SEQ ID NO: 408 and SEQ ID NO: 565, respectively;
xcv. SEQ ID NO: 409 and SEQ ID NO: 566, respectively;
xcvi. SEQ ID NO: 410 and SEQ ID NO: 567, respectively;
xcvii. SEQ ID NO: 411 and SEQ ID NO: 568, respectively;
xcviii. SEQ ID NO: 412 and SEQ ID NO: 569, respectively;
xcix. SEQ ID NO: 413 and SEQ ID NO: 570, respectively;
c. SEQ ID NO: 414 and SEQ ID NO: 571, respectively;
ci. SEQ ID NO: 415 and SEQ ID NO: 572, respectively;
cii. SEQ ID NO: 416 and SEQ ID NO: 573, respectively;
ciii. SEQ ID NO: 417 and SEQ ID NO: 574, respectively;
civ. SEQ ID NO: 418 and SEQ ID NO: 575, respectively;
cv. SEQ ID NO: 419 and SEQ ID NO: 576, respectively;
cvi. SEQ ID NO: 420 and SEQ ID NO: 577, respectively;
cvii. SEQ ID NO: 421 and SEQ ID NO: 578, respectively;
cviii. SEQ ID NO: 422 and SEQ ID NO: 579, respectively;
cix. SEQ ID NO: 423 and SEQ ID NO: 580, respectively;
cx. SEQ ID NO: 424 and SEQ ID NO: 581, respectively;
cxi. SEQ ID NO: 425 and SEQ ID NO: 582, respectively;
cxii. SEQ ID NO: 426 and SEQ ID NO: 583, respectively;
cxiii. SEQ ID NO: 427 and SEQ ID NO: 584, respectively;
cxiv. SEQ ID NO: 428 and SEQ ID NO: 585, respectively;
cxv. SEQ ID NO: 429 and SEQ ID NO: 586, respectively;
cxvi. SEQ ID NO: 430 and SEQ ID NO: 587, respectively;
cxvii. SEQ ID NO: 431 and SEQ ID NO: 588, respectively;
cxviii. SEQ ID NO: 432 and SEQ ID NO: 589, respectively;
cxix. SEQ ID NO: 433 and SEQ ID NO: 590, respectively;
cxx. SEQ ID NO: 434 and SEQ ID NO: 591, respectively;
cxxi. SEQ ID NO: 435 and SEQ ID NO: 592, respectively;
cxxii. SEQ ID NO: 436 and SEQ ID NO: 593, respectively;
cxxiii. SEQ ID NO: 437 and SEQ ID NO: 594, respectively;
cxxiv. SEQ ID NO: 438 and SEQ ID NO: 595, respectively;
cxxv. SEQ ID NO: 439 and SEQ ID NO: 596, respectively;
cxxvi. SEQ ID NO: 440 and SEQ ID NO: 597, respectively;
cxxvii. SEQ ID NO: 441 and SEQ ID NO: 598, respectively;
cxxviii. SEQ ID NO: 442 and SEQ ID NO: 599, respectively;
cxxix. SEQ ID NO: 443 and SEQ ID NO: 600, respectively;
cxxx. SEQ ID NO: 444 and SEQ ID NO: 601, respectively;
cxxxi. SEQ ID NO: 445 and SEQ ID NO: 602, respectively;
cxxxii. SEQ ID NO: 446 and SEQ ID NO: 603, respectively;
cxxxiii. SEQ ID NO: 447 and SEQ ID NO: 604, respectively;
cxxxiv. SEQ ID NO: 448 and SEQ ID NO: 605, respectively;
cxxxv. SEQ ID NO: 449 and SEQ ID NO: 606, respectively;
cxxxvi. SEQ ID NO: 450 and SEQ ID NO: 607, respectively;
cxxxvii. SEQ ID NO: 451 and SEQ ID NO: 608, respectively;
cxxxviii. SEQ ID NO: 452 and SEQ ID NO: 609, respectively;
cxxxix. SEQ ID NO: 453 and SEQ ID NO: 610, respectively;
cxl. SEQ ID NO: 454 and SEQ ID NO: 611, respectively;
cxli. SEQ ID NO: 455 and SEQ ID NO: 612, respectively;
cxlii. SEQ ID NO: 456 and SEQ ID NO: 613, respectively;
cxliii. SEQ ID NO: 457 and SEQ ID NO: 614, respectively;
cxliv. SEQ ID NO: 458 and SEQ ID NO: 615, respectively;
cxlv. SEQ ID NO: 459 and SEQ ID NO: 616, respectively;
cxlvi. SEQ ID NO: 460 and SEQ ID NO: 617, respectively;
cxlvii. SEQ ID NO: 461 and SEQ ID NO: 618, respectively;
cxlviii. SEQ ID NO: 462 and SEQ ID NO: 619, respectively;
cxlix. SEQ ID NO: 463 and SEQ ID NO: 620, respectively;
cl. SEQ ID NO: 464 and SEQ ID NO: 621, respectively;
cli. SEQ ID NO: 465 and SEQ ID NO: 622, respectively;
clii. SEQ ID NO: 466 and SEQ ID NO: 623, respectively;
cliii. SEQ ID NO: 467 and SEQ ID NO: 624, respectively;
cliv. SEQ ID NO: 468 and SEQ ID NO: 625, respectively;
clv. SEQ ID NO: 469 and SEQ ID NO: 626, respectively;
clvi. SEQ ID NO: 470 and SEQ ID NO: 627, respectively; and
clvii. SEQ ID NO: 471 and SEQ ID NO: 628, respectively,
wherein the antigen-binding protein (e.g., the anti-GIPR antibody) preferably comprises one or more cysteine amino acid substitution(s) at one or more position(s) selected from 88 of the light chain, 384 of the heavy chain, or 487 of the heavy chain, according to AHo numbering.

In some embodiments, the light chain comprises the amino acid sequence of SEQ ID NO: 388 and the heavy chain comprises the amino acid sequence of SEQ ID NO: 1571; or the light chain comprises the amino acid sequence of SEQ ID NO: 455 and the heavy chain comprises the amino acid sequence of SEQ ID NO: 1572; or the light chain comprises the amino acid sequence of SEQ ID NO: 389 and the heavy chain comprises the amino acid sequence of SEQ ID NO: 1573; or the light chain comprises the amino acid sequence of SEQ ID NO: 455 and the heavy chain comprises the amino acid sequence of SEQ ID NO: 1574; or the light chain comprises the amino acid sequence of SEQ ID NO: 1575 and the heavy chain comprises the amino acid sequence of SEQ ID NO: 612.

In some embodiments, the light chain comprises the amino acid sequence of SEQ ID NO: 388 and the heavy chain comprises the amino acid sequence of SEQ ID NO: 1571.

In some embodiments, the antigen-binding protein comprises the CDRs, variable domains, and/or full length sequences listed in Tables 9-17 and is a monoclonal antibody, a chimeric antibody, a humanized antibody, a human antibody, a multispecific antibody, or an antibody fragment of the foregoing. In some embodiments, the antigen-binding protein is a monoclonal antibody. In some embodiments, the antigen-binding protein is a chimeric antibody. In some embodiments, the antigen-binding protein is a humanized antibody. In some embodiments, the antigen-binding protein is a human antibody. In some embodiments, the antigen-binding protein is a multispecific antibody. In some embodiments, the antigen-binding protein is an antibody fragment, optionally a Fab fragment, a Fab′ fragment, an F(ab′)₂ fragment, an Fv fragment, a diabody, or a scFv.

In some embodiments, the antigen-binding protein (e.g., the anti-GIPR antibody) competes with one of the antibodies of Table 9 for specific binding to a human GIPR (e.g., SEQ ID NO: 1577, SEQ ID NO: 1578, or SEQ ID NO: 1579). In some embodiments, the antigen-binding protein binds to the same epitope as one of the one of the antibodies of Table 9, or to an overlapping epitope.

In some embodiments, the antigen-binding protein (e.g., the anti-GIPR antibody) competes with iPS:361192 for specific binding to a human GIPR (e.g., SEQ ID NO: 1577, SEQ ID NO: 1578, or SEQ ID NO: 1579). In some embodiments, the antigen-binding protein binds to the same epitope as iPS:361192, or to an overlapping epitope.

In some embodiments, the antigen-binding protein (e.g., the anti-GIPR antibody) competes with iPS:336067 for specific binding to a human GIPR (e.g., SEQ ID NO: 1577, SEQ ID NO: 1578, or SEQ ID NO: 1579). In some embodiments, the antigen-binding protein binds to the same epitope as iPS:336067, or to an overlapping epitope.

In some embodiments, the antigen-binding protein is a monoclonal antibody. Monoclonal antibodies can be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Myeloma cells for use in hybridoma-producing fusion procedures preferably are non-antibody-producing, have high fusion efficiency, and possess enzyme deficiencies that render them incapable of growing in certain selective media which support the growth of only the desired fused cells (hybridomas). Examples of suitable cell lines for use in mouse fusions include, but are not limited to, Sp-20, P3-X63/Ag8, P3-X63-Ag8.653, NS1/1.Ag 4 1, Sp210-Ag14, FO, NSO/U, MPC-11, MPC11-X45-GTG 1.7 and 5194/5XXO Bul; examples of cell lines used in rat fusions include, but are not limited to, R210.RCY3, Y3-Ag 1.2.3, IR983F and 4B210. Other cell lines useful for cell fusions include, but are not limited to, U-266, GM1500-GRG2, LICR-LON-HMy2 and UC729-6.

In some instances, a hybridoma cell line can be produced by immunizing an animal (e.g., a transgenic animal having human immunoglobulin sequences) with a GIPR immunogen; harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds a GIPR polypeptide. Monoclonal antibodies secreted by a hybridoma cell line can be purified using any technique known in the art.

In some embodiments, the antigen-binding protein is a chimeric antibody, which is an antibody composed of protein segments from different antibodies that are covalently joined to produce functional immunoglobulin light or heavy chains or immunologically functional portions thereof. Generally, a portion of the heavy chain and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. For methods relating to chimeric antibodies, see, for example, U.S. Pat. No. 4,816,567; and Morrison et al., 1985, Proc. Natl. Acad. Sci. USA 81:6851-6855, which are hereby incorporated by reference. CDR grafting is described, for example, in U.S. Pat. Nos. 6,180,370, 5,693,762, 5,693,761, 5,585,089, and 5,530,101.

Generally, the goal of making a chimeric antibody is to create a chimera in which the number of amino acids from the intended patient species is maximized. One non-limiting example is the “CDR-grafted” antibody, in which the antibody comprises one or more complementarity determining regions (CDRs) from a particular species or belonging to a particular antibody class or subclass, while the remainder of the antibody chain(s) is/are identical with or homologous to a corresponding sequence in antibodies derived from another species or belonging to another antibody class or subclass. For use in humans, the variable region or selected CDRs from a rodent antibody can be grafted into a human antibody, replacing the naturally-occurring variable regions or CDRs of the human antibody.

In some embodiments, the chimeric antibody is a humanized antibody. Generally, a humanized antibody is produced from a monoclonal antibody raised initially in a non-human animal. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent variable region for the corresponding regions of a human antibody (see, e.g., U.S. Pat. Nos. 5,585,089, and 5,693,762; Jones et al., 1986, Nature 321:522-525; Riechmann et al., 1988, Nature 332:323-27; Verhoeyen et al., 1988, Science 239:1534-1536).

In some embodiments, the CDRs of the light and heavy chain variable regions of the anti-GIPR antibodies provided herein are grafted to framework regions (FRs) from antibodies from the same, or a different, phylogenetic species. For example, the CDRs of the heavy and light chain variable regions V_{H}1, V_{H}2, V_{H}3, V_{H}4, V_{H}5, V_{H}6, V_{H}7, V_{H}8, V_{H}9, V_{H}10, V_{H}11, V_{H}12 and/or V_{L}1, and V_{L}2 can be grafted to consensus human FRs. To create consensus human FRs, FRs from several human heavy chain or light chain amino acid sequences may be aligned to identify a consensus amino acid sequence. In other embodiments, the FRs of a heavy chain or light chain disclosed herein are replaced with the FRs from a different heavy chain or light chain. In one aspect, rare amino acids in the FRs of the heavy and light chains of GIPR antibodies are not replaced, while the rest of the FR amino acids are replaced. A “rare amino acid” is a specific amino acid that is in a position in which this particular amino acid is not usually found in an FR. Alternatively, the grafted variable regions from the one heavy or light chain may be used with a constant region that is different from the constant region of that particular heavy or light chain as disclosed herein. In other embodiments, the grafted variable regions are part of a single chain Fv antibody.

In some embodiments, constant regions from species other than human can be used along with the human variable region(s) to produce hybrid antibodies.

In some embodiments, the antigen-binding protein is a human antibody. Methods are available for making fully human antibodies specific for a given antigen without exposing human beings to the antigen. One specific means provided for implementing the production of fully human antibodies is the “humanization” of the mouse humoral immune system. Introduction of human immunoglobulin (Ig) loci into mice in which the endogenous Ig genes have been inactivated is one means of producing fully human monoclonal antibodies (mAbs) in mouse, an animal that can be immunized with any desirable antigen. Using fully human antibodies can minimize the immunogenic and allergic responses that can sometimes be caused by administering mouse or mouse-derived mAbs to humans as therapeutic agents.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, e.g., Jakobovits et al., 1993, Proc. Natl. Acad. Sci. USA 90:2551-2555; Jakobovits et al., 1993, Nature 362:255-258; and Bruggermann et al., 1993, Year in Immunol. 7:33. In one example of such a method, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then cross-bred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, WO96/33735 and WO94/02602. Additional methods relating to transgenic mice for making human antibodies are described in U.S. Pat. Nos. 5,545,807; 6,713,610; 6,673,986; 6,162,963; 5,545,807; 6,300,129; 6,255,458; 5,877,397; 5,874,299 and 5,545,806; in PCT publications WO91/10741, WO90/04036, and in EP 546073B1 and EP 546073A1.

Fully human antibodies can also be derived from phage-display libraries (as disclosed in Hoogenboom et al., 1991, J. Mol. Biol. 227:381; and Marks et al., 1991, J. Mol. Biol. 222:581). Phage display techniques mimic immune selection through the display of antibody repertoires on the surface of filamentous bacteriophage, and subsequent selection of phage by their binding to an antigen of choice. One such technique is described in PCT Publication No. WO 99/10494 (hereby incorporated by reference).

In some embodiments, the antigen-binding protein is a mimetic (e.g., a “peptide mimetic” or “peptidomimetic”) based upon the variable region domains and CDRs that are described herein. These analogs can be peptides, non-peptides, or combinations of peptide and non-peptide regions. Fauchere, 1986, Adv. Drug Res. 15:29; Veber and Freidinger, 1985, TINS p. 392; and Evans et al., 1987, J. Med. Chem. 30:1229, which are incorporated herein by reference for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics are proteins that are structurally similar to an antibody displaying a desired biological activity, such as the ability to specifically bind GIPR, but have one or more peptide linkages optionally replaced by a linkage selected from: —CH₂NH—, —CH₂S—, —CH₂—CH₂—, —CH—CH— (cis and trans), —COCH₂—, —CH(OH)CH₂—, and —CH₂SO—, by methods well known in the art. In some embodiments, systematic substitution of one or more amino acids of a consensus sequence with a D-amino acid of the same type (e.g., D-lysine in place of L-lysine) can be used to generate more stable proteins. In addition, constrained peptides comprising a consensus sequence or a substantially identical consensus sequence variation can be generated by methods known in the art (Rizo and Gierasch, 1992, Ann. Rev. Biochem. 61:387), incorporated herein by reference), for example, by adding internal cysteine residues capable of forming intramolecular disulfide bridges which cyclize the peptide.

In some embodiments, the antigen-binding protein may be derivatized. In some embodiments, the derivatized antigen-binding protein comprises a molecule that imparts a desired property to the antigen-binding protein, such as increased half-life in a particular use. The derivatized antigen-binding protein can comprise, for example, a detectable (or labeling) moiety (e.g., a radioactive, colorimetric, antigenic, or enzymatic molecule, a detectable bead (such as, e.g., a magnetic or electrodense (e.g., gold) bead), or a molecule that binds to another molecule (e.g., biotin or streptavidin)), a therapeutic or diagnostic moiety (e.g., a radioactive, cytotoxic, or pharmaceutically active moiety), or a molecule that increases the suitability of the antigen-binding protein for a particular use (e.g., administration to a subject, such as a human subject, or other in vivo or in vitro uses). Non-limiting examples of molecules that can be used to derivatize an antigen-binding protein include albumin (e.g., human serum albumin) and polyethylene glycol (PEG). Albumin-linked and PEGylated derivatives of antigen-binding proteins can be prepared using techniques well known in the art. In some embodiments, the antigen-binding protein may be conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, polypropylene glycol homopolymers, polypropylene oxide/ethylene oxide co-polymers, polyoxyethylated polyols, and polyvinyl alcohols.

Antigen-binding proteins used in the conjugates described herein may be prepared by any of a number of conventional techniques. For example, antigen-binding proteins can be produced by recombinant expression systems. See, e.g., Monoclonal Antibodies, Hybridomas: A New Dimension in Biological Analyses, Kennet et al. (eds.) Plenum Press, New York (1980); and Antibodies: A Laboratory Manual, Harlow and Lane (eds.), Cold Spring Harbor Laboratory Press, Cold Spring Harbor, N.Y. (1988).

In some embodiments, antigen-binding proteins (e.g., anti-GIPR antibodies) can be expressed in hybridoma cell lines or in cell lines other than hybridomas. Expression constructs encoding the antigen-binding proteins (e.g., anti-GIPR antibodies) can be used to transform a mammalian, insect or microbial host cell. Transformation can be performed using any known method for introducing polynucleotides into a host cell, including, for example, packaging the polynucleotide in a virus or bacteriophage and transducing a host cell with the construct by transfection procedures known in the art, as exemplified by U.S. Pat. Nos. 4,399,216; 4,912,040; 4,740,461; 4,959,455. The choice of transformation procedure used will depend upon which type of host cell is being transformed. Methods for introduction of heterologous polynucleotides into mammalian cells are known in the art and include, but are not limited to, dextran-mediated transfection, calcium phosphate precipitation, polybrene mediated transfection, protoplast fusion, electroporation, encapsulation of the polynucleotide(s) in liposomes, mixing nucleic acid with positively-charged lipids, and direct microinjection of the DNA into nuclei.

Recombinant expression constructs typically comprise a nucleic acid molecule encoding a polypeptide comprising one or more of the following: one or more CDRs; a light chain constant region; a light chain variable region; a heavy chain constant region (e.g., C_{H}1, C_{H}2 and/or C_{H}3); and/or another scaffold portion of an antigen-binding protein (e.g., an anti-GIPR antibody). These nucleic acid sequences are inserted into an appropriate expression vector using standard ligation techniques. In some embodiments, the heavy or light chain constant region is appended to the C-terminus of the anti-GIPR specific heavy or light chain variable region and is ligated into an expression vector. The vector is typically selected to be functional in the particular host cell employed (i.e., the vector is compatible with the host cell machinery, permitting amplification, and/or expression of the gene can occur). In some embodiments, vectors are used that employ protein-fragment complementation assays using protein reporters, such as dihydrofolate reductase (see, for example, U.S. Pat. No. 6,270,964, which is hereby incorporated by reference). Expression vectors can be purchased, for example, from Invitrogen Life Technologies or BD Biosciences. Other useful vectors for cloning and expressing antigen-binding proteins (e.g., anti-GIPR antibodies) include, but are not limited to, those described in Bianchi and McGrew, 2003, Biotech. Biotechnol. Bioeng. 84:439-44, which is hereby incorporated by reference. Additional suitable expression vectors are discussed, for example, in Methods Enzymol., vol. 185 (D. V. Goeddel, ed.), 1990, New York: Academic Press.

Typically, expression vectors used in any of the host cells will contain sequences for plasmid maintenance and for cloning and expression of exogenous nucleotide sequences. Such sequences, collectively referred to as “flanking sequences,” can include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Optionally, the vector may contain a “tag”-encoding sequence, i.e., an oligonucleotide molecule located at the 5′ or 3′ end of the GIPR antigen binding protein coding sequence; the oligonucleotide sequence encodes polyHis (such as hexaHis), or another “tag” such as FLAG®, HA (hemagglutinin influenza virus), or myc, for which commercially available antibodies exist. This tag is typically fused to the polypeptide upon expression of the polypeptide, and can serve as a means for affinity purification or detection of the antigen-binding protein from the host cell. Affinity purification can be accomplished, for example, by column chromatography using antibodies against the tag as an affinity matrix. Optionally, the tag can subsequently be removed from the purified antigen-binding protein by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), synthetic, or native. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors can be obtained by methods known in the art. Typically, flanking sequences will have been previously identified by mapping and/or by restriction endonuclease digestion and can thus be isolated from the proper tissue source using the appropriate restriction endonucleases. In some cases, the full nucleotide sequence of a flanking sequence may be known. The flanking sequence may be synthesized using conventional methods for nucleic acid synthesis or cloning.

Whether all or only a portion of the flanking sequence is known, it may be obtained using polymerase chain reaction (PCR) and/or by screening a genomic library with a suitable probe such as an oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, CA), or other methods known to the skilled artisan. The selection of suitable enzymes to accomplish this purpose will be readily apparent to one of ordinary skill in the art.

An origin of replication is typically a part of commercially available prokaryotic expression vectors; the origin of replication aids in the amplification of the vector in a host cell. If the vector of choice does not contain an origin of replication site, one may be chemically synthesized based on a known sequence and ligated into the vector. For example, the origin of replication from the plasmid pBR322 (New England Biolabs, Beverly, MA) is suitable for most gram-negative bacteria and various viral origins (e.g., SV40, polyoma, adenovirus, vesicular stomatitus virus (VSV), or papillomaviruses, such as, e.g., HPV or BPV). Generally, the origin of replication component is not needed for mammalian expression vectors (for example, the SV40 origin is often used only because it also contains the virus early promoter).

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using conventional methods for nucleic acid synthesis.

A selectable marker gene encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene may also be used for selection in both prokaryotic and eukaryotic host cells.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include dihydrofolate reductase (DHFR) and promoterless thyrnidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene, such as an antigen binding protein that binds GIPR polypeptide. As a result, increased quantities of a polypeptide such as an antigen binding protein are synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed.

In some cases, such as where glycosylation is desired in a eukaryotic host cell expression system, one may manipulate the various pre- or pro-sequences to improve glycosylation or yield. For example, one may alter the peptidase cleavage site of a particular signal peptide, or add prosequences, which also may affect glycosylation. The final protein product may have, in the −1 position (relative to the first amino acid of the mature protein), one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired polypeptide, if the enzyme cuts at such area within the mature polypeptide.

Expression and cloning will typically contain a promoter that is recognized by the host organism and operably linked to the molecule encoding the antigen-binding protein. Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe a gene to which they are operably linked, that is, with little or no control over gene expression. A large number of promoters, recognized by a variety of potential host cells, are known. A suitable promoter is operably linked to the DNA encoding heavy chain or light chain comprising a GIPR antigen binding protein by removing the promoter from the source DNA by restriction enzyme digestion and inserting the desired promoter sequence into the vector.

Suitable promoters for use with yeast hosts are also known in the art. Yeast enhancers are advantageously used with yeast promoters. Suitable promoters for use with mammalian host cells are known and include, but are not limited to, those obtained from the genomes of viruses such as polyoma virus, fowlpox virus, adenovirus (such as, e.g., Adenovirus 2), bovine papilloma virus, avian sarcoma virus, cytomegalovirus, retroviruses, hepatitis-B virus, and Simian Virus 40 (SV40). Other suitable mammalian promoters include, but are not limited to, heterologous mammalian promoters, for example, heat-shock promoters and the actin promoter.

An enhancer sequence may be inserted into the vector to increase transcription of DNA encoding light chain or heavy chain comprising a GIPR antigen binding protein by higher eukaryotes. Enhancers are cis-acting elements of DNA, usually about 10-300 bp in length, which act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Alternatively, an enhancer from a virus can be used. The SV40 enhancer, the cytomegalovirus early promoter enhancer, the polyoma enhancer, and adenovirus enhancers known in the art are non-limiting examples of enhancing elements for the activation of eukaryotic promoters. While an enhancer may be positioned in the vector either 5′ or 3′ to a coding sequence, it is typically located at a site 5′ from the promoter. A sequence encoding an appropriate native or heterologous signal sequence (leader sequence or signal peptide) can be incorporated into an expression vector, e.g., to promote extracellular secretion of the antibody. The choice of signal peptide or leader depends on the type of host cells in which the antibody is to be produced, and a heterologous signal sequence can replace the native signal sequence. Non-limiting examples of signal peptides that are functional in mammalian host cells include the following: the signal sequence for interleukin-7 (IL-7) described in U.S. Pat. No. 4,965,195; the signal sequence for interleukin-2 receptor described in Cosman et al., 1984, Nature 312:768; the interleukin-4 receptor signal peptide described in EP Patent No. 0367 566; the type I interleukin-1 receptor signal peptide described in U.S. Pat. No. 4,968,607; the type II interleukin-1 receptor signal peptide described in EP Patent No. 0 460 846.

In one embodiment the leader sequence comprises SEQ ID NO: 1585 (MDMRVPAQLL GLLLLWLRGA RC). In another embodiment the leader sequence comprises SEQ ID NO: 1586 (MAWALLLLTL LTQGTGSWA).

Expression vectors may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are known to one skilled in the art.

After the vector has been constructed and a nucleic acid molecule encoding a light chain, a heavy chain, or a light chain and a heavy chain has been inserted into the proper site of the vector, the completed vector may be inserted into a suitable host cell for amplification and/or polypeptide expression. The transformation of an expression vector for an antigen-binding protein into a selected host cell may be accomplished by conventional methods including, but not limited to, transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are known to the skilled artisan, and are set forth, for example, in Sambrook et al., 2001, supra.

A host cell, when cultured under appropriate conditions, synthesizes an antigen-binding protein that can subsequently be collected from the culture medium (if the host cell secretes it into the medium) or directly from the host cell producing it (if it is not secreted). The selection of an appropriate host cell will depend upon various factors, such as, e.g., desired expression levels, polypeptide modifications that are desirable or necessary for activity (such as, e.g., glycosylation or phosphorylation), and ease of folding into a biologically active molecule.

Mammalian cell lines available as hosts for expression are known in the art and include, but are not limited to, immortalized cell lines available from the American Type Culture Collection (ATCC), including, but not limited to, Chinese hamster ovary (CHO) cells, HeLa cells, baby hamster kidney (BHK) cells, monkey kidney cells (COS), human hepatocellular carcinoma cells (e.g., Hep G2), and a number of other cell lines. In some embodiments, a host cell line can be selected by determining which cell lines have high expression levels and constitutively produce the desired antigen-binding protein (e.g., the desired anti-GIPR antibody). In some embodiments, a cell line from the B cell lineage that does not make its own antibody but has a capacity to make and secrete a heterologous antibody can be selected.

Additionally, in some embodiments, a mammalian cell line modified to provide for reduced cleavage of peptides that will be conjugated to polypeptides expressed by the cells (e.g., a cathepsin D knock-out cell line) can be used. In some embodiments, a mammalian cell line (e.g., a CHO cell line) in which both alleles of cathepsin D are knocked out (e.g., using CRISPR or zinc-finger technology) can be used. An example of a cathepsin D knock-out cell that can be used to produce an antibody-peptide conjugate of the disclosure is described in WO 2023/086790, which is incorporated by reference herein.

In some embodiments, the first polypeptide that agonizes a glucagon receptor is glucagon or a glucagon analog. In some embodiments, the first polypeptide is glucagon. In some embodiments, the first polypeptide is human glucagon. In some embodiments, the first polypeptide is a glucagon analog. In some embodiments, the first polypeptide is a human glucagon analog.

In some embodiments, the first polypeptide comprises the amino acid sequence of SEQ ID NO: 1576. In some embodiments, the first polypeptide consists of the amino acid sequence of SEQ ID NO: 1576.

In some embodiments, the first polypeptide has at least 68%, at least 72%, at least 75%, at least 79%, at least 82%, at least 86%, at least 89%, at least 93%, or at least 96% sequence identity to SEQ ID NO: 1576. In some embodiments, the first polypeptide has at least 68% sequence identity to SEQ ID NO: 1576. In some embodiments, the first polypeptide has at least 72% sequence identity to SEQ ID NO: 1576. In some embodiments, the first polypeptide has at least 75% sequence identity to SEQ ID NO: 1576. In some embodiments, the first polypeptide has at least 79% sequence identity to SEQ ID NO: 1576. In some embodiments, the first polypeptide has at least 82% sequence identity to SEQ ID NO: 1576. In some embodiments, the first polypeptide has at least 86% sequence identity to SEQ ID NO: 1576. In some embodiments, the first polypeptide has at least 89% sequence identity to SEQ ID NO: 1576. In some embodiments, the first polypeptide has at least 93% sequence identity to SEQ ID NO: 1576. In some embodiments, the first polypeptide has at least 96% sequence identity to SEQ ID NO: 1576.

In some embodiments, the first polypeptide comprises an amino acid sequence having at most nine (e.g., zero, one, two, three, four, five, six, seven, eight, or nine) amino acid modifications relative to SEQ ID NO: 1576. In some embodiments, the first polypeptide comprises an amino acid sequence having at most eight amino acid modifications relative to SEQ ID NO: 1576. In some embodiments, the first polypeptide comprises an amino acid sequence having at most seven amino acid modifications relative to SEQ ID NO: 1576. In some embodiments, the first polypeptide comprises an amino acid sequence having at most six amino acid modifications relative to SEQ ID NO: 1576. In some embodiments, the first polypeptide comprises an amino acid sequence having at most five amino acid modifications relative to SEQ ID NO: 1576. In some embodiments, the first polypeptide comprises an amino acid sequence having at most four amino acid modifications relative to SEQ ID NO: 1576. In some embodiments, the first polypeptide comprises an amino acid sequence having at most three amino acid modifications relative to SEQ ID NO: 1576. In some embodiments, the first polypeptide comprises an amino acid sequence having at most two amino acid modifications relative to SEQ ID NO: 1576. In some embodiments, the first polypeptide comprises an amino acid sequence having at most one amino acid modification relative to SEQ ID NO: 1576.

In some embodiments, each amino acid modification, if any, is an amino acid substitution. In some embodiments, each amino acid modification, if any, is a conservative amino acid substitution. In some embodiments, each amino acid modification, if any, is a conservative amino acid substitution listed in Table 1.

In some embodiments, the at most one amino acid modification is an amino acid deletion. In some embodiments, the at most one amino acid modification is an amino acid addition.

Potential amino acid modifications relative to SEQ ID NO: 1576, include, but are not limited to, S2s, S16Aib, S16Q, S16E, R17K, R18A, R18Y, R18F, D21E, Q24D, Q24E, Q24K, W25-5-BrW, M27L, M27E, N28A, N28D, N28K, N28Q, and T29D.

In some embodiments, the first polypeptide is an glucagon receptor agonist polypeptide provided herein.

In some embodiments, the first polypeptide comprises an amino acid sequence with between three and nine modifications relative to SEQ ID NO: 1576, wherein the modifications are selected from:
tyrosine and phenylalanine at position 1;
d-serine, 2-aminoisobutyric acid, and d-threonine at position 2;
glutamic acid at position 3;
histidine at position 7;
tryptophan at position 10;
glutamic acid at position 15;
2-aminoisobutyric acid, glutamine, homophenylalanine, and glutamic acid at position 16;
lysine, citrulline, glutamine, and alanine at position 17;
2-naphthylalanine, L-4,4′-biphenylalanine, alanine, citrulline, and lysine at position 18;
4-chloro-L-phenylalanine, alanine, d-glutamine, homoserine, histidine, arginine, and glutamic acid at position 20;
glutamic acid, citrulline, and d-aspartic acid at position 21;
tryptophan and β-cyclohexyl-L-alanine at position 22;
aspartic acid, lysine, alanine, 2-aminoisobutyric acid, glycine, histidine, asparagine, threonine, d-glutamine, glutamic acid, arginine, phenylalanine, leucine, serine, tyrosine, valine, isoleucine, homoserine, and 2,3-diaminopropionic acid at position 24;
5-bromo-L-tryptophan, tyrosine, L-beta-homotryptophan, 5-methoxy-L-tryptophan, 5-methyl-L-tryptophan, 6-bromo-L-tryptophan, 6-chloro-L-tryptophan, 6-methyl-L-tryptophan, and 7-bromo-L-tryptophan at position 25;
leucine, glutamic acid, and L-α-aminoadipic acid at position 27;
lysine, aspartic acid, serine, 6-azido-L-lysine, glutamic acid, and alanine at position 28;
glutamic acid, serine, aspartic acid, and alanine at position 29;
an additional amino acid at position 30, wherein the additional amino acid is lysine; and
an additional amino acid at position 31, wherein the additional amino acid is lysine.

In some embodiments, the modifications comprise a d-serine at position 2, a 2-aminoisobutyric acid at position 16, and between one and seven other modifications at position 1, 3, 7, 10, 15, 17, 18, 20, 21, 22, 24, 25, 26, 27, 28, 29, 30, or 31 (e.g., at position 17, 21, 24, 25, 27, 28, or 29) as described above. In some embodiments, the modifications are selected from:
lysine at position 17;
glutamic acid at position 21;
lysine, alanine, and glutamic acid at position 24;
5-bromo-L-tryptophan at position 25;
leucine and glutamic acid at position 27;
lysine, aspartic acid, and glutamic acid at position 28; and
glutamic acid and serine at position 29.

In some embodiments, the modifications comprise a d-serine at position 2, a 2-aminoisobutyric acid at position 16, and between one and six other modifications selected from:
lysine at position 17;
glutamic acid at position 21;
lysine, alanine, and glutamic acid at position 24;
leucine and glutamic acid at position 27;
lysine, aspartic acid, and glutamic acid at position 28; and
glutamic acid and serine at position 29.

In some embodiments, the modifications comprise a d-serine at position 2, a 2-aminoisobutyric acid at position 16, and one or two other modifications selected from:
lysine at position 24; and
lysine and glutamic acid at position 28.

In some embodiments, the modifications comprise d-serine at position 2, 2-aminoisobutyric acid at position 16, and lysine at position 24. In some embodiments, the modifications comprise d-serine at position 2, 2-aminoisobutyric acid at position 16, lysine at position 24, and glutamic acid at position 28.

In some embodiments, the modifications comprise d-serine at position 2, 2-aminoisobutyric acid at position 16, and lysine at position 28.

In some embodiments, the modifications comprise tyrosine at position 1, d-serine at position 2, and 2-aminoisobutyric acid at position 16. In some embodiments, the modifications comprise phenylalanine at position 1, d-serine at position 2, and 2-aminoisobutyric acid at position 16.

In some embodiments, the modifications comprise d-serine at position 2, glutamic acid at position 3, and 2-aminoisobutyric acid at position 16.

In some embodiments, the modifications comprise d-serine at position 2, histidine at position 7, and 2-aminoisobutyric acid at position 16.

In some embodiments, the modifications comprise d-serine at position 2, tryptophan at position 10, and 2-aminoisobutyric acid at position 16.

In some embodiments, the modifications comprise d-serine at position 2, glutamic acid at position 15, and 2-aminoisobutyric acid at position 16.

In some embodiments, the modifications comprise d-serine at position 2, 2-aminoisobutyric acid at position 16, and lysine at position 17. In some embodiments, the modifications comprise d-serine at position 2, 2-aminoisobutyric acid at position 16, and citrulline at position 17. In some embodiments, the modifications comprise d-serine at position 2, 2-aminoisobutyric acid at position 16, and glutamine at position 17. In some embodiments, the modifications comprise d-serine at position 2, 2-aminoisobutyric acid at position 16, and alanine at position 17.

In some embodiments, the modifications comprise d-serine at position 2, 2-aminoisobutyric acid at position 16, and 2-naphthylalanine at position 18. In some embodiments, the modifications comprise d-serine at position 2, 2-aminoisobutyric acid at position 16, and L-4,4′-biphenylalanine at position 18. In some embodiments, the modifications comprise d-serine at position 2, 2-aminoisobutyric acid at position 16, and alanine at position 18. In some embodiments, the modifications comprise d-serine at position 2, 2-aminoisobutyric acid at position 16, and citrulline at position 18. In some embodiments, the modifications comprise d-serine at position 2, 2-aminoisobutyric acid at position 16, and lysine at position 18.

In some embodiments, the modifications comprise d-serine at position 2, 2-aminoisobutyric acid at position 16, and 4-chloro-L-phenylalanine at position 20. In some embodiments, the modifications comprise d-serine at position 2, 2-aminoisobutyric acid at position 16, and alanine at position 20. In some embodiments, the modifications comprise d-serine at position 2, 2-aminoisobutyric acid at position 16, and d-glutamine at position 20. In some embodiments, the modifications comprise d-serine at position 2, 2-aminoisobutyric acid at position 16, and homoserine at position 20. In some embodiments, the modifications comprise d-serine at position 2, 2-aminoisobutyric acid at position 16, and histidine at position 20. In some embodiments, the modifications comprise d-serine at position 2, 2-aminoisobutyric acid at position 16, and arginine at position 20. In some embodiments, the modifications comprise d-serine at position 2, 2-aminoisobutyric acid at position 16, and glutamic acid at position 20.

In some embodiments, the modifications comprise d-serine at position 2, 2-aminoisobutyric acid at position 16, and glutamic acid at position 21. In some embodiments, the modifications comprise d-serine at position 2, 2-aminoisobutyric acid at position 16, and citrulline at position 21. In some embodiments, the modifications comprise d-serine at position 2, 2-aminoisobutyric acid at position 16, and d-aspartic acid at position 21.

In some embodiments, the modifications comprise d-serine at position 2, 2-aminoisobutyric acid at position 16, and tryptophan at position 22. In some embodiments, the modifications comprise d-serine at position 2, 2-aminoisobutyric acid at position 16, and β-cyclohexyl-L-alanine at position 22.

In some embodiments, the modifications comprise d-serine at position 2, 2-aminoisobutyric acid at position 16, and aspartic acid at position 24. In some embodiments, the modifications comprise d-serine at position 2, 2-aminoisobutyric acid at position 16, and lysine at position 24. In some embodiments, the modifications comprise d-serine at position 2, 2-aminoisobutyric acid at position 16, and alanine at position 24. In some embodiments, the modifications comprise d-serine at position 2, 2-aminoisobutyric acid at position 16, and 2-aminoisobutyric acid at position 24. In some embodiments, the modifications comprise d-serine at position 2, 2-aminoisobutyric acid at position 16, and glycine at position 24. In some embodiments, the modifications comprise d-serine at position 2, 2-aminoisobutyric acid at position 16, and histidine at position 24. In some embodiments, the modifications comprise d-serine at position 2, 2-aminoisobutyric acid at position 16, and asparagine at position 24. In some embodiments, the modifications comprise d-serine at position 2, 2-aminoisobutyric acid at position 16, and threonine at position 24. In some embodiments, the modifications comprise d-serine at position 2, 2-aminoisobutyric acid at position 16, and d-glutamine at position 24. In some embodiments, the modifications comprise d-serine at position 2, 2-aminoisobutyric acid at position 16, and glutamic acid at position 24. In some embodiments, the modifications comprise d-serine at position 2, 2-aminoisobutyric acid at position 16, and arginine at position 24. In some embodiments, the modifications comprise d-serine at position 2, 2-aminoisobutyric acid at position 16, and phenylalanine at position 24. In some embodiments, the modifications comprise d-serine at position 2, 2-aminoisobutyric acid at position 16, and leucine at position 24. In some embodiments, the modifications comprise d-serine at position 2, 2-aminoisobutyric acid at position 16, and serine at position 24. In some embodiments, the modifications comprise d-serine at position 2, 2-aminoisobutyric acid at position 16, and tyrosine at position 24. In some embodiments, the modifications comprise d-serine at position 2, 2-aminoisobutyric acid at position 16, and valine at position 24. In some embodiments, the modifications comprise d-serine at position 2, 2-aminoisobutyric acid at position 16, and isoleucine at position 24. In some embodiments, the modifications comprise d-serine at position 2, 2-aminoisobutyric acid at position 16, and homoserine at position 24. In some embodiments, the modifications comprise d-serine at position 2, 2-aminoisobutyric acid at position 16, and 2,3-diaminopropionic acid at position 24.

In some embodiments, the modifications comprise d-serine at position 2, 2-aminoisobutyric acid at position 16, and 5-bromo-L-tryptophan at position 25. In some embodiments, the modifications comprise d-serine at position 2, 2-aminoisobutyric acid at position 16, and tyrosine at position 25. In some embodiments, the modifications comprise d-serine at position 2, 2-aminoisobutyric acid at position 16, and L-beta-homotryptophan at position 25. In some embodiments, the modifications comprise d-serine at position 2, 2-aminoisobutyric acid at position 16, and 5-methoxy-L-tryptophan at position 25. In some embodiments, the modifications comprise d-serine at position 2, 2-aminoisobutyric acid at position 16, and 5-methyl-L-tryptophan at position 25. In some embodiments, the modifications comprise d-serine at position 2, 2-aminoisobutyric acid at position 16, and 6-bromo-L-tryptophan at position 25. In some embodiments, the modifications comprise d-serine at position 2, 2-aminoisobutyric acid at position 16, and 6-chloro-L-tryptophan at position 25. In some embodiments, the modifications comprise d-serine at position 2, 2-aminoisobutyric acid at position 16, and 6-methyl-L-tryptophan at position 25. In some embodiments, the modifications comprise d-serine at position 2, 2-aminoisobutyric acid at position 16, and 7-bromo-L-tryptophan at position 25.

In some embodiments, the modifications comprise d-serine at position 2, 2-aminoisobutyric acid at position 16, and alanine at position 24.

In some embodiments, the modifications comprise d-serine at position 2, 2-aminoisobutyric acid at position 16, and leucine at position 27. In some embodiments, the modifications comprise d-serine at position 2, 2-aminoisobutyric acid at position 16, and glutamic acid at position 27. In some embodiments, the modifications comprise d-serine at position 2, 2-aminoisobutyric acid at position 16, and L-α-aminoadipic acid at position 27.

In some embodiments, the modifications comprise d-serine at position 2, 2-aminoisobutyric acid at position 16, and lysine at position 28. In some embodiments, the modifications comprise d-serine at position 2, 2-aminoisobutyric acid at position 16, and aspartic acid at position 28. In some embodiments, the modifications comprise d-serine at position 2, 2-aminoisobutyric acid at position 16, and serine at position 28. In some embodiments, the modifications comprise d-serine at position 2, 2-aminoisobutyric acid at position 16, and 6-azido-L-lysine at position 28. In some embodiments, the modifications comprise d-serine at position 2, 2-aminoisobutyric acid at position 16, and glutamic acid at position 28. In some embodiments, the modifications comprise d-serine at position 2, 2-aminoisobutyric acid at position 16, and alanine at position 28.

In some embodiments, the modifications comprise d-serine at position 2, 2-aminoisobutyric acid at position 16, and glutamic acid at position 29. In some embodiments, the modifications comprise d-serine at position 2, 2-aminoisobutyric acid at position 16, and serine at position 29. In some embodiments, the modifications comprise d-serine at position 2, 2-aminoisobutyric acid at position 16, and aspartic acid at position 29. In some embodiments, the modifications comprise d-serine at position 2, 2-aminoisobutyric acid at position 16, and alanine at position 29.

In some embodiments, the modifications comprise d-serine at position 2, 2-aminoisobutyric acid at position 16, and an additional amino acid at position 30, wherein the additional amino acid is lysine.

In some embodiments, the modifications comprise d-serine at position 2, 2-aminoisobutyric acid at position 16, and an additional amino acid at position 31, wherein the additional amino acid is lysine. In some embodiments, the modifications comprise d-serine at position 2, 2-aminoisobutyric acid at position 16, an additional amino acid at position 30, and an additional amino acid at position 31, wherein the additional amino acid at position 30 and the additional amino acid at position 31 are both lysine.

In some embodiments, the first polypeptide comprises at least 25 amino acids, wherein: the first polypeptide comprises 5-bromo-tryptophan at position 25; and the first polypeptide has at least 79% (e.g., at least 82%, at least 86%, at least 89%, at least 93%, at least 96%) sequence identity to the amino acid sequence of SEQ ID NO: 1587. In some embodiments, the first polypeptide comprises SEQ ID NO: 1587. In some embodiments, the first polypeptide consists of SEQ ID NO: 1587.

In some embodiments, the first polypeptide comprises at least 28 amino acids, wherein: the first polypeptide comprises 2-aminoisobutyric acid at position 16 and lysine, serine, or aspartic acid at position 28; and the first polypeptide has at least 89% (e.g., at least 93%, at least 96%) sequence identity to the amino acid sequence of SEQ ID NO: 1596. In some embodiments, the first polypeptide comprises SEQ ID NO: 1596. In some embodiments, the first polypeptide consists of SEQ ID NO: 1596.

In some embodiments, the first polypeptide comprises at least 28 amino acids, wherein: the first polypeptide comprises 2-aminoisobutyric acid at position 16, leucine at position 27, and lysine at position 28; and the first polypeptide has at least 89% (e.g., at least 93%, at least 96%) sequence identity to the amino acid sequence of SEQ ID NO: 1615. In some embodiments, the first polypeptide comprises SEQ ID NO: 1615. In some embodiments, the first polypeptide consists of SEQ ID NO: 1615.

In some embodiments, the first polypeptide comprises at least 28 amino acids, wherein: the first polypeptide comprises 2-aminoisobutyric acid at position 16, aspartic acid at position 24, and lysine at position 28; and the first polypeptide has at least 89% (e.g., at least 93%, at least 96%) sequence identity to the amino acid sequence of SEQ ID NO: 1626.

In some embodiments, the first polypeptide comprises SEQ ID NO: 1626. In some embodiments, the first polypeptide consists of SEQ ID NO: 1626.

In some embodiments, the first polypeptide comprises at least 28 amino acids, wherein the first polypeptide comprises a d-serine at position 2 and a 2-aminoisobutyric acid at position 16; and the first polypeptide has at least 89% (e.g., at least 93%, at least 96%) sequence identity to the amino acid sequence of SEQ ID NO: 1822. In some embodiments, the first polypeptide comprises SEQ ID NO: 1822. In some embodiments, the first polypeptide consists of SEQ ID NO: 1822.

In some embodiments, the first polypeptide comprises the amino acid sequence of any one of SEQ ID NOs: 1587-1627, 1747-1840, 1859-1862, or 1879-1881. In some embodiments, the first polypeptide comprises the amino acid sequence of any one of SEQ ID NOs: 1587-1627 or 1747. In some embodiments, the first polypeptide comprises the amino acid sequence of any one of SEQ ID NOs: 1748-1840, 1859-1862, or 1879-1881.

In some embodiments, the first polypeptide comprises the amino acid sequence of any one of SEQ ID NOs: 1587-1627.

In some embodiments, the first polypeptide comprises the amino acid sequence of any one of SEQ ID NOs: 1587, 1592, 1615, 1626, 1818, 1822, 1825, or 1826. In some embodiments, the first polypeptide comprises the amino acid sequence of any one of SEQ ID NOs: 1587, 1592, 1615, or 1626. In some embodiments, the first polypeptide comprises the amino acid sequence of any one of SEQ ID NOs: 1626, 1818, 1822, 1825, or 1826.

In some embodiments, the first polypeptide comprises the amino acid sequence of any one of SEQ ID NOs: 1596, 1597, 1615, 1626, 1818, 1822, 1825, or 1826. In some embodiments, the first polypeptide comprises the amino acid sequence of any one of SEQ ID NOs: 1596, 1597, 1615, or 1626.

In some embodiments, the first polypeptide consists of the amino acid sequence of any one of SEQ ID NOs: 1587-1627, 1747-1840, 1859-1862, or 1879-1881. In some embodiments, the first polypeptide consists of the amino acid sequence of any one of SEQ ID NOs: 1587-1627 or 1747. In some embodiments, the first polypeptide consists of the amino acid sequence of any one of SEQ ID NOs: 1748-1840, 1859-1862, or 1879-1881.

In some embodiments, the first polypeptide consists of the amino acid sequence of any one of SEQ ID NOs: 1587-1627.

In some embodiments, the first polypeptide consists of the amino acid sequence of any one of SEQ ID NOs: 1587, 1592, 1615, 1626, 1818, 1822, 1825, or 1826. In some embodiments, the first polypeptide consists of the amino acid sequence of any one of SEQ ID NOs: 1587, 1592, 1615, or 1626. In some embodiments, the first polypeptide consists of the amino acid sequence of any one of SEQ ID NOs: 1626, 1818, 1822, 1825, or 1826.

In some embodiments, the first polypeptide consists of the amino acid sequence of any one of SEQ ID NOs: 1596, 1597, 1615, or 1626.

In some embodiments, the first polypeptide comprises the amino acid sequence of SEQ ID NO: 1587. In some embodiments, the first polypeptide comprises the amino acid sequence of SEQ ID NO: 1588. In some embodiments, the first polypeptide comprises the amino acid sequence of SEQ ID NO: 1589. In some embodiments, the first polypeptide comprises the amino acid sequence of SEQ ID NO: 1590. In some embodiments, the first polypeptide comprises the amino acid sequence of SEQ ID NO: 1591. In some embodiments, the first polypeptide comprises the amino acid sequence of SEQ ID NO: 1592. In some embodiments, the first polypeptide comprises the amino acid sequence of SEQ ID NO: 1593. In some embodiments, the first polypeptide comprises the amino acid sequence of SEQ ID NO: 1594. In some embodiments, the first polypeptide comprises the amino acid sequence of SEQ ID NO: 1595. In some embodiments, the first polypeptide comprises the amino acid sequence of SEQ ID NO: 1596. In some embodiments, the first polypeptide comprises the amino acid sequence of SEQ ID NO: 1597. In some embodiments, the first polypeptide comprises the amino acid sequence of SEQ ID NO: 1598. In some embodiments, the first polypeptide comprises the amino acid sequence of SEQ ID NO: 1599. In some embodiments, the first polypeptide comprises the amino acid sequence of SEQ ID NO: 1600. In some embodiments, the first polypeptide comprises the amino acid sequence of SEQ ID NO: 1601. In some embodiments, the first polypeptide comprises the amino acid sequence of SEQ ID NO: 1602. In some embodiments, the first polypeptide comprises the amino acid sequence of SEQ ID NO: 1603. In some embodiments, the first polypeptide comprises the amino acid sequence of SEQ ID NO: 1604. In some embodiments, the first polypeptide comprises the amino acid sequence of SEQ ID NO: 1605. In some embodiments, the first polypeptide comprises the amino acid sequence of SEQ ID NO: 1606. In some embodiments, the first polypeptide comprises the amino acid sequence of SEQ ID NO: 1607. In some embodiments, the first polypeptide comprises the amino acid sequence of SEQ ID NO: 1608. In some embodiments, the first polypeptide comprises the amino acid sequence of SEQ ID NO: 1609. In some embodiments, the first polypeptide comprises the amino acid sequence of SEQ ID NO: 1610. In some embodiments, the first polypeptide comprises the amino acid sequence of SEQ ID NO: 1611. In some embodiments, the first polypeptide comprises the amino acid sequence of SEQ ID NO: 1612. In some embodiments, the first polypeptide comprises the amino acid sequence of SEQ ID NO: 1613. In some embodiments, the first polypeptide comprises the amino acid sequence of SEQ ID NO: 1614. In some embodiments, the first polypeptide comprises the amino acid sequence of SEQ ID NO: 1615. In some embodiments, the first polypeptide comprises the amino acid sequence of SEQ ID NO: 1616. In some embodiments, the first polypeptide comprises the amino acid sequence of SEQ ID NO: 1617. In some embodiments, the first polypeptide comprises the amino acid sequence of SEQ ID NO: 1618. In some embodiments, the first polypeptide comprises the amino acid sequence of SEQ ID NO: 1619. In some embodiments, the first polypeptide comprises the amino acid sequence of SEQ ID NO: 1620. In some embodiments, the first polypeptide comprises the amino acid sequence of SEQ ID NO: 1621. In some embodiments, the first polypeptide comprises the amino acid sequence of SEQ ID NO: 1622. In some embodiments, the first polypeptide comprises the amino acid sequence of SEQ ID NO: 1623. In some embodiments, the first polypeptide comprises the amino acid sequence of SEQ ID NO: 1624. In some embodiments, the first polypeptide comprises the amino acid sequence of SEQ ID NO: 1625. In some embodiments, the first polypeptide comprises the amino acid sequence of SEQ ID NO: 1626. In some embodiments, the first polypeptide comprises the amino acid sequence of SEQ ID NO: 1627.

In some embodiments, the first polypeptide comprises the amino acid sequence of SEQ ID NO: 1747. In some embodiments, the first polypeptide comprises the amino acid sequence of SEQ ID NO: 1748. In some embodiments, the first polypeptide comprises the amino acid sequence of SEQ ID NO: 1749. In some embodiments, the first polypeptide comprises the amino acid sequence of SEQ ID NO: 1750. In some embodiments, the first polypeptide comprises the amino acid sequence of SEQ ID NO: 1751. In some embodiments, the first polypeptide comprises the amino acid sequence of SEQ ID NO: 1752. In some embodiments, the first polypeptide comprises the amino acid sequence of SEQ ID NO: 1753. In some embodiments, the first polypeptide comprises the amino acid sequence of SEQ ID NO: 1754. In some embodiments, the first polypeptide comprises the amino acid sequence of SEQ ID NO: 1755. In some embodiments, the first polypeptide comprises the amino acid sequence of SEQ ID NO: 1756. In some embodiments, the first polypeptide comprises the amino acid sequence of SEQ ID NO: 1757. In some embodiments, the first polypeptide comprises the amino acid sequence of SEQ ID NO: 1758. In some embodiments, the first polypeptide comprises the amino acid sequence of SEQ ID NO: 1759. In some embodiments, the first polypeptide comprises the amino acid sequence of SEQ ID NO: 1760. In some embodiments, the first polypeptide comprises the amino acid sequence of SEQ ID NO: 1761. In some embodiments, the first polypeptide comprises the amino acid sequence of SEQ ID NO: 1762. In some embodiments, the first polypeptide comprises the amino acid sequence of SEQ ID NO: 1763. In some embodiments, the first polypeptide comprises the amino acid sequence of SEQ ID NO: 1764. In some embodiments, the first polypeptide comprises the amino acid sequence of SEQ ID NO: 1765. In some embodiments, the first polypeptide comprises the amino acid sequence of SEQ ID NO: 1766. In some embodiments, the first polypeptide comprises the amino acid sequence of SEQ ID NO: 1767. In some embodiments, the first polypeptide comprises the amino acid sequence of SEQ ID NO: 1768. In some embodiments, the first polypeptide comprises the amino acid sequence of SEQ ID NO: 1769. In some embodiments, the first polypeptide comprises the amino acid sequence of SEQ ID NO: 1770. In some embodiments, the first polypeptide comprises the amino acid sequence of SEQ ID NO: 1771. In some embodiments, the first polypeptide comprises the amino acid sequence of SEQ ID NO: 1772. In some embodiments, the first polypeptide comprises the amino acid sequence of SEQ ID NO: 1773. In some embodiments, the first polypeptide comprises the amino acid sequence of SEQ ID NO: 1774. In some embodiments, the first polypeptide comprises the amino acid sequence of SEQ ID NO: 1775. In some embodiments, the first polypeptide comprises the amino acid sequence of SEQ ID NO: 1776. In some embodiments, the first polypeptide comprises the amino acid sequence of SEQ ID NO: 1777. In some embodiments, the first polypeptide comprises the amino acid sequence of SEQ ID NO: 1778. In some embodiments, the first polypeptide comprises the amino acid sequence of SEQ ID NO: 1779. In some embodiments, the first polypeptide comprises the amino acid sequence of SEQ ID NO: 1780. In some embodiments, the first polypeptide comprises the amino acid sequence of SEQ ID NO: 1781. In some embodiments, the first polypeptide comprises the amino acid sequence of SEQ ID NO: 1782. In some embodiments, the first polypeptide comprises the amino acid sequence of SEQ ID NO: 1783. In some embodiments, the first polypeptide comprises the amino acid sequence of SEQ ID NO: 1784. In some embodiments, the first polypeptide comprises the amino acid sequence of SEQ ID NO: 1785. In some embodiments, the first polypeptide comprises the amino acid sequence of SEQ ID NO: 1786. In some embodiments, the first polypeptide comprises the amino acid sequence of SEQ ID NO: 1787. In some embodiments, the first polypeptide comprises the amino acid sequence of SEQ ID NO: 1788. In some embodiments, the first polypeptide comprises the amino acid sequence of SEQ ID NO: 1789. In some embodiments, the first polypeptide comprises the amino acid sequence of SEQ ID NO: 1790. In some embodiments, the first polypeptide comprises the amino acid sequence of SEQ ID NO: 1791. In some embodiments, the first polypeptide comprises the amino acid sequence of SEQ ID NO: 1792. In some embodiments, the first polypeptide comprises the amino acid sequence of SEQ ID NO: 1793. In some embodiments, the first polypeptide comprises the amino acid sequence of SEQ ID NO: 1794. In some embodiments, the first polypeptide comprises the amino acid sequence of SEQ ID NO: 1795. In some embodiments, the first polypeptide comprises the amino acid sequence of SEQ ID NO: 1796. In some embodiments, the first polypeptide comprises the amino acid sequence of SEQ ID NO: 1797. In some embodiments, the first polypeptide comprises the amino acid sequence of SEQ ID NO: 1798. In some embodiments, the first polypeptide comprises the amino acid sequence of SEQ ID NO: 1799. In some embodiments, the first polypeptide comprises the amino acid sequence of SEQ ID NO: 1800. In some embodiments, the first polypeptide comprises the amino acid sequence of SEQ ID NO: 1801. In some embodiments, the first polypeptide comprises the amino acid sequence of SEQ ID NO: 1802. In some embodiments, the first polypeptide comprises the amino acid sequence of SEQ ID NO: 1803. In some embodiments, the first polypeptide comprises the amino acid sequence of SEQ ID NO: 1804. In some embodiments, the first polypeptide comprises the amino acid sequence of SEQ ID NO: 1805. In some embodiments, the first polypeptide comprises the amino acid sequence of SEQ ID NO: 1806. In some embodiments, the first polypeptide comprises the amino acid sequence of SEQ ID NO: 1807. In some embodiments, the first polypeptide comprises the amino acid sequence of SEQ ID NO: 1808. In some embodiments, the first polypeptide comprises the amino acid sequence of SEQ ID NO: 1809. In some embodiments, the first polypeptide comprises the amino acid sequence of SEQ ID NO: 1810. In some embodiments, the first polypeptide comprises the amino acid sequence of SEQ ID NO: 1811. In some embodiments, the first polypeptide comprises the amino acid sequence of SEQ ID NO: 1812. In some embodiments, the first polypeptide comprises the amino acid sequence of SEQ ID NO: 1813. In some embodiments, the first polypeptide comprises the amino acid sequence of SEQ ID NO: 1814. In some embodiments, the first polypeptide comprises the amino acid sequence of SEQ ID NO: 1815. In some embodiments, the first polypeptide comprises the amino acid sequence of SEQ ID NO: 1816. In some embodiments, the first polypeptide comprises the amino acid sequence of SEQ ID NO: 1817. In some embodiments, the first polypeptide comprises the amino acid sequence of SEQ ID NO: 1818. In some embodiments, the first polypeptide comprises the amino acid sequence of SEQ ID NO: 1819. In some embodiments, the first polypeptide comprises the amino acid sequence of SEQ ID NO: 1820. In some embodiments, the first polypeptide comprises the amino acid sequence of SEQ ID NO: 1821. In some embodiments, the first polypeptide comprises the amino acid sequence of SEQ ID NO: 1822. In some embodiments, the first polypeptide comprises the amino acid sequence of SEQ ID NO: 1823. In some embodiments, the first polypeptide comprises the amino acid sequence of SEQ ID NO: 1824. In some embodiments, the first polypeptide comprises the amino acid sequence of SEQ ID NO: 1825. In some embodiments, the first polypeptide comprises the amino acid sequence of SEQ ID NO: 1826. In some embodiments, the first polypeptide comprises the amino acid sequence of SEQ ID NO: 1827. In some embodiments, the first polypeptide comprises the amino acid sequence of SEQ ID NO: 1828. In some embodiments, the first polypeptide comprises the amino acid sequence of SEQ ID NO: 1829. In some embodiments, the first polypeptide comprises the amino acid sequence of SEQ ID NO: 1830. In some embodiments, the first polypeptide comprises the amino acid sequence of SEQ ID NO: 1831. In some embodiments, the first polypeptide comprises the amino acid sequence of SEQ ID NO: 1832. In some embodiments, the first polypeptide comprises the amino acid sequence of SEQ ID NO: 1833. In some embodiments, the first polypeptide comprises the amino acid sequence of SEQ ID NO: 1834. In some embodiments, the first polypeptide comprises the amino acid sequence of SEQ ID NO: 1835. In some embodiments, the first polypeptide comprises the amino acid sequence of SEQ ID NO: 1836. In some embodiments, the first polypeptide comprises the amino acid sequence of SEQ ID NO: 1837. In some embodiments, the first polypeptide comprises the amino acid sequence of SEQ ID NO: 1838. In some embodiments, the first polypeptide comprises the amino acid sequence of SEQ ID NO: 1839. In some embodiments, the first polypeptide comprises the amino acid sequence of SEQ ID NO: 1840. In some embodiments, the first polypeptide comprises the amino acid sequence of SEQ ID NO: 1859. In some embodiments, the first polypeptide comprises the amino acid sequence of SEQ ID NO: 1860. In some embodiments, the first polypeptide comprises the amino acid sequence of SEQ ID NO: 1861. In some embodiments, the first polypeptide comprises the amino acid sequence of SEQ ID NO: 1862. In some embodiments, the first polypeptide consists of the amino acid sequence of SEQ ID NO: 1879. In some embodiments, the first polypeptide consists of the amino acid sequence of SEQ ID NO: 1880. In some embodiments, the first polypeptide consists of the amino acid sequence of SEQ ID NO: 1881.

In some embodiments, the first polypeptide consists of the amino acid sequence of SEQ ID NO: 1587. In some embodiments, the first polypeptide consists of the amino acid sequence of SEQ ID NO: 1588. In some embodiments, the first polypeptide consists of the amino acid sequence of SEQ ID NO: 1589. In some embodiments, the first polypeptide consists of the amino acid sequence of SEQ ID NO: 1590. In some embodiments, the first polypeptide consists of the amino acid sequence of SEQ ID NO: 1591. In some embodiments, the first polypeptide consists of the amino acid sequence of SEQ ID NO: 1592. In some embodiments, the first polypeptide consists of the amino acid sequence of SEQ ID NO: 1593. In some embodiments, the first polypeptide consists of the amino acid sequence of SEQ ID NO: 1594. In some embodiments, the first polypeptide consists of the amino acid sequence of SEQ ID NO: 1595. In some embodiments, the first polypeptide consists of the amino acid sequence of SEQ ID NO: 1596. In some embodiments, the first polypeptide consists of the amino acid sequence of SEQ ID NO: 1597. In some embodiments, the first polypeptide consists of the amino acid sequence of SEQ ID NO: 1598. In some embodiments, the first polypeptide consists of the amino acid sequence of SEQ ID NO: 1599. In some embodiments, the first polypeptide consists of the amino acid sequence of SEQ ID NO: 1600. In some embodiments, the first polypeptide consists of the amino acid sequence of SEQ ID NO: 1601. In some embodiments, the first polypeptide consists of the amino acid sequence of SEQ ID NO: 1602. In some embodiments, the first polypeptide consists of the amino acid sequence of SEQ ID NO: 1603. In some embodiments, the first polypeptide consists of the amino acid sequence of SEQ ID NO: 1604. In some embodiments, the first polypeptide consists of the amino acid sequence of SEQ ID NO: 1605. In some embodiments, the first polypeptide consists of the amino acid sequence of SEQ ID NO: 1606. In some embodiments, the first polypeptide consists of the amino acid sequence of SEQ ID NO: 1607. In some embodiments, the first polypeptide consists of the amino acid sequence of SEQ ID NO: 1608. In some embodiments, the first polypeptide consists of the amino acid sequence of SEQ ID NO: 1609. In some embodiments, the first polypeptide consists of the amino acid sequence of SEQ ID NO: 1610. In some embodiments, the first polypeptide consists of the amino acid sequence of SEQ ID NO: 1611. In some embodiments, the first polypeptide consists of the amino acid sequence of SEQ ID NO: 1612. In some embodiments, the first polypeptide consists of the amino acid sequence of SEQ ID NO: 1613. In some embodiments, the first polypeptide consists of the amino acid sequence of SEQ ID NO: 1614. In some embodiments, the first polypeptide consists of the amino acid sequence of SEQ ID NO: 1615. In some embodiments, the first polypeptide consists of the amino acid sequence of SEQ ID NO: 1616. In some embodiments, the first polypeptide consists of the amino acid sequence of SEQ ID NO: 1617. In some embodiments, the first polypeptide consists of the amino acid sequence of SEQ ID NO: 1618. In some embodiments, the first polypeptide consists of the amino acid sequence of SEQ ID NO: 1619. In some embodiments, the first polypeptide consists of the amino acid sequence of SEQ ID NO: 1620. In some embodiments, the first polypeptide consists of the amino acid sequence of SEQ ID NO: 1621. In some embodiments, the first polypeptide consists of the amino acid sequence of SEQ ID NO: 1622. In some embodiments, the first polypeptide consists of the amino acid sequence of SEQ ID NO: 1623. In some embodiments, the first polypeptide consists of the amino acid sequence of SEQ ID NO: 1624. In some embodiments, the first polypeptide consists of the amino acid sequence of SEQ ID NO: 1625. In some embodiments, the first polypeptide consists of the amino acid sequence of SEQ ID NO: 1626. In some embodiments, the first polypeptide consists of the amino acid sequence of SEQ ID NO: 1627.

In some embodiments, the first polypeptide consists of the amino acid sequence of SEQ ID NO: 1747. In some embodiments, the first polypeptide consists of the amino acid sequence of SEQ ID NO: 1748. In some embodiments, the first polypeptide consists of the amino acid sequence of SEQ ID NO: 1749. In some embodiments, the first polypeptide consists of the amino acid sequence of SEQ ID NO: 1750. In some embodiments, the first polypeptide consists of the amino acid sequence of SEQ ID NO: 1751. In some embodiments, the first polypeptide consists of the amino acid sequence of SEQ ID NO: 1752. In some embodiments, the first polypeptide consists of the amino acid sequence of SEQ ID NO: 1753. In some embodiments, the first polypeptide consists of the amino acid sequence of SEQ ID NO: 1754. In some embodiments, the first polypeptide consists of the amino acid sequence of SEQ ID NO: 1755. In some embodiments, the first polypeptide consists of the amino acid sequence of SEQ ID NO: 1756. In some embodiments, the first polypeptide consists of the amino acid sequence of SEQ ID NO: 1757. In some embodiments, the first polypeptide consists of the amino acid sequence of SEQ ID NO: 1758. In some embodiments, the first polypeptide consists of the amino acid sequence of SEQ ID NO: 1759. In some embodiments, the first polypeptide consists of the amino acid sequence of SEQ ID NO: 1760. In some embodiments, the first polypeptide consists of the amino acid sequence of SEQ ID NO: 1761. In some embodiments, the first polypeptide consists of the amino acid sequence of SEQ ID NO: 1762. In some embodiments, the first polypeptide consists of the amino acid sequence of SEQ ID NO: 1763. In some embodiments, the first polypeptide consists of the amino acid sequence of SEQ ID NO: 1764. In some embodiments, the first polypeptide consists of the amino acid sequence of SEQ ID NO: 1765. In some embodiments, the first polypeptide consists of the amino acid sequence of SEQ ID NO: 1766. In some embodiments, the first polypeptide consists of the amino acid sequence of SEQ ID NO: 1767. In some embodiments, the first polypeptide consists of the amino acid sequence of SEQ ID NO: 1768. In some embodiments, the first polypeptide consists of the amino acid sequence of SEQ ID NO: 1769. In some embodiments, the first polypeptide consists of the amino acid sequence of SEQ ID NO: 1770. In some embodiments, the first polypeptide consists of the amino acid sequence of SEQ ID NO: 1771. In some embodiments, the first polypeptide consists of the amino acid sequence of SEQ ID NO: 1772. In some embodiments, the first polypeptide consists of the amino acid sequence of SEQ ID NO: 1773. In some embodiments, the first polypeptide consists of the amino acid sequence of SEQ ID NO: 1774. In some embodiments, the first polypeptide consists of the amino acid sequence of SEQ ID NO: 1775. In some embodiments, the first polypeptide consists of the amino acid sequence of SEQ ID NO: 1776. In some embodiments, the first polypeptide consists of the amino acid sequence of SEQ ID NO: 1777. In some embodiments, the first polypeptide consists of the amino acid sequence of SEQ ID NO: 1778. In some embodiments, the first polypeptide consists of the amino acid sequence of SEQ ID NO: 1779. In some embodiments, the first polypeptide consists of the amino acid sequence of SEQ ID NO: 1780. In some embodiments, the first polypeptide consists of the amino acid sequence of SEQ ID NO: 1781. In some embodiments, the first polypeptide consists of the amino acid sequence of SEQ ID NO: 1782. In some embodiments, the first polypeptide consists of the amino acid sequence of SEQ ID NO: 1783. In some embodiments, the first polypeptide consists of the amino acid sequence of SEQ ID NO: 1784. In some embodiments, the first polypeptide consists of the amino acid sequence of SEQ ID NO: 1785. In some embodiments, the first polypeptide consists of the amino acid sequence of SEQ ID NO: 1786. In some embodiments, the first polypeptide consists of the amino acid sequence of SEQ ID NO: 1787. In some embodiments, the first polypeptide consists of the amino acid sequence of SEQ ID NO: 1788. In some embodiments, the first polypeptide consists of the amino acid sequence of SEQ ID NO: 1789. In some embodiments, the first polypeptide consists of the amino acid sequence of SEQ ID NO: 1790. In some embodiments, the first polypeptide consists of the amino acid sequence of SEQ ID NO: 1791. In some embodiments, the first polypeptide consists of the amino acid sequence of SEQ ID NO: 1792. In some embodiments, the first polypeptide consists of the amino acid sequence of SEQ ID NO: 1793. In some embodiments, the first polypeptide consists of the amino acid sequence of SEQ ID NO: 1794. In some embodiments, the first polypeptide consists of the amino acid sequence of SEQ ID NO: 1795. In some embodiments, the first polypeptide consists of the amino acid sequence of SEQ ID NO: 1796. In some embodiments, the first polypeptide consists of the amino acid sequence of SEQ ID NO: 1797. In some embodiments, the first polypeptide consists of the amino acid sequence of SEQ ID NO: 1798. In some embodiments, the first polypeptide consists of the amino acid sequence of SEQ ID NO: 1799. In some embodiments, the first polypeptide consists of the amino acid sequence of SEQ ID NO: 1800. In some embodiments, the first polypeptide consists of the amino acid sequence of SEQ ID NO: 1801. In some embodiments, the first polypeptide consists of the amino acid sequence of SEQ ID NO: 1802. In some embodiments, the first polypeptide consists of the amino acid sequence of SEQ ID NO: 1803. In some embodiments, the first polypeptide consists of the amino acid sequence of SEQ ID NO: 1804. In some embodiments, the first polypeptide consists of the amino acid sequence of SEQ ID NO: 1805. In some embodiments, the first polypeptide consists of the amino acid sequence of SEQ ID NO: 1806. In some embodiments, the first polypeptide consists of the amino acid sequence of SEQ ID NO: 1807. In some embodiments, the first polypeptide consists of the amino acid sequence of SEQ ID NO: 1808. In some embodiments, the first polypeptide consists of the amino acid sequence of SEQ ID NO: 1809. In some embodiments, the first polypeptide consists of the amino acid sequence of SEQ ID NO: 1810. In some embodiments, the first polypeptide consists of the amino acid sequence of SEQ ID NO: 1811. In some embodiments, the first polypeptide consists of the amino acid sequence of SEQ ID NO: 1812. In some embodiments, the first polypeptide consists of the amino acid sequence of SEQ ID NO: 1813. In some embodiments, the first polypeptide consists of the amino acid sequence of SEQ ID NO: 1814. In some embodiments, the first polypeptide consists of the amino acid sequence of SEQ ID NO: 1815. In some embodiments, the first polypeptide consists of the amino acid sequence of SEQ ID NO: 1816. In some embodiments, the first polypeptide consists of the amino acid sequence of SEQ ID NO: 1817. In some embodiments, the first polypeptide consists of the amino acid sequence of SEQ ID NO: 1818. In some embodiments, the first polypeptide consists of the amino acid sequence of SEQ ID NO: 1819. In some embodiments, the first polypeptide consists of the amino acid sequence of SEQ ID NO: 1820. In some embodiments, the first polypeptide consists of the amino acid sequence of SEQ ID NO: 1821. In some embodiments, the first polypeptide consists of the amino acid sequence of SEQ ID NO: 1822. In some embodiments, the first polypeptide consists of the amino acid sequence of SEQ ID NO: 1823. In some embodiments, the first polypeptide consists of the amino acid sequence of SEQ ID NO: 1824. In some embodiments, the first polypeptide consists of the amino acid sequence of SEQ ID NO: 1825. In some embodiments, the first polypeptide consists of the amino acid sequence of SEQ ID NO: 1826. In some embodiments, the first polypeptide consists of the amino acid sequence of SEQ ID NO: 1827. In some embodiments, the first polypeptide consists of the amino acid sequence of SEQ ID NO: 1828. In some embodiments, the first polypeptide consists of the amino acid sequence of SEQ ID NO: 1829. In some embodiments, the first polypeptide consists of the amino acid sequence of SEQ ID NO: 1830. In some embodiments, the first polypeptide consists of the amino acid sequence of SEQ ID NO: 1831. In some embodiments, the first polypeptide consists of the amino acid sequence of SEQ ID NO: 1832. In some embodiments, the first polypeptide consists of the amino acid sequence of SEQ ID NO: 1833. In some embodiments, the first polypeptide consists of the amino acid sequence of SEQ ID NO: 1834. In some embodiments, the first polypeptide consists of the amino acid sequence of SEQ ID NO: 1835. In some embodiments, the first polypeptide consists of the amino acid sequence of SEQ ID NO: 1836. In some embodiments, the first polypeptide consists of the amino acid sequence of SEQ ID NO: 1837. In some embodiments, the first polypeptide consists of the amino acid sequence of SEQ ID NO: 1838. In some embodiments, the first polypeptide consists of the amino acid sequence of SEQ ID NO: 1839. In some embodiments, the first polypeptide consists of the amino acid sequence of SEQ ID NO: 1840. In some embodiments, the first polypeptide consists of the amino acid sequence of SEQ ID NO: 1859. In some embodiments, the first polypeptide consists of the amino acid sequence of SEQ ID NO: 1860. In some embodiments, the first polypeptide consists of the amino acid sequence of SEQ ID NO: 1861. In some embodiments, the first polypeptide consists of the amino acid sequence of SEQ ID NO: 1862. In some embodiments, the first polypeptide consists of the amino acid sequence of SEQ ID NO: 1879. In some embodiments, the first polypeptide consists of the amino acid sequence of SEQ ID NO: 1880. In some embodiments, the first polypeptide consists of the amino acid sequence of SEQ ID NO: 1881.

In some embodiments, the first polypeptide is conjugated to the antigen-binding protein (e.g., the anti-GIPR antibody) through a linker moiety, such as, e.g., a linker moiety described below. In some embodiments, the first polypeptide is conjugated to the antigen-binding protein (e.g., the anti-GIPR antibody) through a first linker polypeptide, such as, e.g., a linker polypeptide described below. In some embodiments, the first linker polypeptide comprises the amino acid sequence of any one of SEQ ID NOs: 1628-1683, 1739-1746, or 1841-1852. In some embodiments, the first linker polypeptide comprises the amino acid sequence of any one of SEQ ID NOs: 1628-1683, 1739, 1850, or 1851. In some embodiments, the first linker polypeptide comprises the amino acid sequence of any one of SEQ ID NOs: 1740-1746, 1841-1849, or 1852. In some embodiments, the first linker polypeptide comprises the amino acid sequence of any one of SEQ ID NOs: 1628-1683. In some embodiments, the first linker polypeptide comprises the amino acid sequence of any one of SEQ ID NOs: 1628-1630. In some embodiments, the first linker polypeptide comprises the amino acid sequence of SEQ ID NO: 1628. In some embodiments, the first linker polypeptide comprises the amino acid sequence of SEQ ID NO: 1629. In some embodiments, the first linker polypeptide comprises the amino acid sequence of SEQ ID NO: 1630.

In some embodiments, a C-terminus of the first polypeptide is covalently linked to a C-terminus of a first linker polypeptide. In some embodiments, the C-terminus of the first polypeptide is derivatized. In some embodiments, the C-terminus of the first linker polypeptide is derivatized. In some embodiments, the C-termini of both the first polypeptide and the first linker polypeptide are derivatized.

In some embodiments, an ε-amino group of a lysine residue of the first polypeptide is covalently linked to a C-terminus of a first linker polypeptide. In some embodiments, a lysine residue of the first polypeptide is covalently linked to a C-terminus of the first linker polypeptide via an amide bond, e.g., an amide bond formed by condensation of an ε-amino group of a lysine residue of the first polypeptide and a carboxyl group of a C-terminus of the first polypeptide linker.

In some embodiments, an N-terminus of the first linker polypeptide is conjugated to a cysteine residue of the antigen-binding protein (e.g., the anti-GIPR antibody). For example, in some embodiments, a derivatized N-terminus of the first linker polypeptide is covalently linked to a cysteine residue of the antigen-binding protein (e.g., the anti-GIPR antibody) via a thioether linkage that comprises a sulfur atom of the cysteine residue. In some embodiments, an acetylated N-terminus of the first linker polypeptide is covalently linked to a cysteine residue of the antigen-binding protein (e.g., the anti-GIPR antibody) via a thioether linkage that comprises a sulfur atom of the cysteine residue.

In some embodiments, the antigen-binding protein is an antibody and the cysteine residue of the antibody that is conjugated to the N-terminus of the first linker polypeptide is at a position selected from the group consisting of 88 of a light chain, 384 of a heavy chain, and 487 of a heavy chain, according to AHo numbering. In some embodiments, the cysteine residue of the antibody that is conjugated to the N-terminus of the first linker polypeptide is at position 384 of a heavy chain, according to AHo numbering.

In some embodiments, an ε-amino group of a lysine residue of the first polypeptide is covalently linked to a C-terminus of a first linker polypeptide, and an N-terminus of the first linker polypeptide is conjugated to a cysteine residue of the antigen-binding protein (e.g., the anti-GIPR antibody). In some embodiments, the antigen-binding protein is an antibody and the cysteine residue of the antibody that is conjugated to the N-terminus of the first linker polypeptide is at a position selected from the group consisting of 88 of a light chain, 384 of a heavy chain, and 487 of a heavy chain, according to AHo numbering. In some embodiments, the cysteine residue of the antibody that is conjugated to the N-terminus of the first linker polypeptide is at position 384 of a heavy chain, according to AHo numbering.

In some embodiments, a C-terminal amino acid residue of the first polypeptide is covalently linked to a N-terminal amino acid residue of a first linker polypeptide. Additionally, in some embodiments, the C-terminus of the first linker polypeptide is derivatized. In some embodiments, the C-terminal amino acid residue of the first linker polypeptide is modified for coupling to a cysteine residue (e.g., is bromoacetylated). In some embodiments, the C-terminal amino acid residue of the first linker polypeptide is a lysine residue. In some embodiments, the C-terminal amino acid residue of the first linker polypeptide is modified for conjugation to a cysteine residue (e.g., bromoacetylated). In some embodiments, the C-terminal amino acid residue of the first linker polypeptide is a bromoacetylated lysine residue.

In some embodiments, a C-terminus of the first linker polypeptide is conjugated to a cysteine residue of the antigen-binding protein (e.g., the anti-GIPR antibody). For example, in some embodiments, a derivatized C-terminus of the first linker polypeptide is covalently linked to a cysteine residue of the antigen-binding protein (e.g., the anti-GIPR antibody) via a thioether linkage that comprises a sulfur atom of the cysteine residue. In some embodiments, an acetylated C-terminus of the first linker polypeptide is covalently linked to a cysteine residue of the antigen-binding protein (e.g., the anti-GIPR antibody) via a thioether linkage that comprises a sulfur atom of the cysteine residue.

In some embodiments, the antigen-binding protein is an antibody and the cysteine residue of the antibody that is conjugated to the C-terminus of the first linker polypeptide is at a position selected from the group consisting of 88 of a light chain, 384 of a heavy chain, and 487 of a heavy chain, according to AHo numbering. In some embodiments, the cysteine residue of the antibody that is conjugated to the C-terminus of the first linker polypeptide is at position 384 of a heavy chain, according to AHo numbering.

In some embodiments, a C-terminal amino acid residue of the first polypeptide is covalently linked to a N-terminal amino acid residue of a first linker polypeptide, and an C-terminus of the first linker polypeptide is conjugated to a cysteine residue of the antigen-binding protein (e.g., the anti-GIPR antibody). In some embodiments, the antigen-binding protein is an antibody and the cysteine residue of the antibody that is conjugated to the C-terminus of the first linker polypeptide is at a position selected from the group consisting of 88 of a light chain, 384 of a heavy chain, and 487 of a heavy chain, according to AHo numbering. In some embodiments, the cysteine residue of the antibody that is conjugated to the C-terminus of the first linker polypeptide is at position 384 of a heavy chain, according to AHo numbering.

In some embodiments, the molecule further comprises a second polypeptide that agonizes a GCGR. The second polypeptide agonist can be a polypeptide agonist as described above (such as, e.g., glucagon (SEQ ID NO: 1576) or a glucagon analog (e.g., a variant of SEQ ID NO: 1576 that includes one or more amino modifications, which may include, but are not limited to, S2s, S16Aib, S16Q, S16E, R17K, R18A, R18Y, R18F, D21E, Q24D, Q24E, Q24K, W25-5-BrW, M27L, M27E, N28A, N28D, N28K, N28Q, and T29D modifications, as described above). In some embodiments, the second polypeptide has the same amino acid sequence as the first polypeptide. In some embodiments, the second polypeptide has a different amino acid sequence than the first polypeptide.

In some embodiments, the second polypeptide comprises the amino acid sequence of any one of SEQ ID NOs: 1587-1627, 1747-1840, 1859-1862, or 1879-1881. In some embodiments, the second polypeptide comprises the amino acid sequence of any one of SEQ ID NOs: 1587-1627 or 1747. In some embodiments, the second polypeptide comprises the amino acid sequence of any one of SEQ ID NOs: 1587-1627. In some embodiments, the second polypeptide comprises the amino acid sequence of any one of SEQ ID NOs: 1747-1840, 1859-1862, or 1879-1881.

In some embodiments, the second polypeptide comprises the amino acid sequence of any one of SEQ ID NOs: 1587, 1592, 1615, 1626, 1818, 1822, 1825, or 1826. In some embodiments, the second polypeptide comprises the amino acid sequence of any one of SEQ ID NOs: 1587, 1592, 1596, 1615, or 1626. In some embodiments, the second polypeptide comprises the amino acid sequence of any one of SEQ ID NOs: 1587, 1592, 1615, or 1626. In some embodiments, the second polypeptide comprises the amino acid sequence of any one of SEQ ID NOs: 1626, 1818, 1822, 1825, or 1826. In some embodiments, the second polypeptide comprises the amino acid sequence of SEQ ID NO: 1587. In some embodiments, the second polypeptide comprises the amino acid sequence of SEQ ID NO: 1592. In some embodiments, the second polypeptide comprises the amino acid sequence of SEQ ID NO: 1596. In some embodiments, the second polypeptide comprises the amino acid sequence of SEQ ID NO: 1615. In some embodiments, the second polypeptide comprises the amino acid sequence of SEQ ID NO: 1626. In some embodiments, the second polypeptide comprises the amino acid sequence of SEQ ID NO: 1818. In some embodiments, the second polypeptide comprises the amino acid sequence of SEQ ID NO: 1822. In some embodiments, the second polypeptide comprises the amino acid sequence of SEQ ID NO: 1825. In some embodiments, the second polypeptide comprises the amino acid sequence of SEQ ID NO: 1826.

In some embodiments, the first polypeptide and the second polypeptide each independently comprise the amino acid sequence of any one of SEQ ID NOs: 1587-1627, 1747-1840, 1859-1862, or 1879-1881.

In some embodiments, the first polypeptide and the second polypeptide each independently comprise the amino acid sequence of any one of SEQ ID NOs: 1587-1627.

In some embodiments, the first polypeptide and the second polypeptide each independently comprise the amino acid sequence of any one of SEQ ID NOs: 1587, 1592, 1596, 1615, 1626, 1818, 1822, 1825, or 1826. In some embodiments, the first polypeptide and the second polypeptide each independently comprise the amino acid sequence of any one of SEQ ID NOs: 1587, 1592, 1615, 1626, 1818, 1822, 1825, or 1826. In some embodiments, the first polypeptide and the second polypeptide each independently comprise the amino acid sequence of any one of SEQ ID NOs: 1626, 1818, 1822, 1825, or 1826.

In some embodiments, the first polypeptide and the second polypeptide each independently comprise the amino acid sequence of any one of SEQ ID NOs: 1587, 1592, 1596, 1615, or 1626.

In some embodiments, the first polypeptide and the second polypeptide both comprise the amino acid sequence of SEQ ID NO: 1587. In some embodiments, the first polypeptide and the second polypeptide both comprise the amino acid sequence of SEQ ID NO: 1592. In some embodiments, the first polypeptide and the second polypeptide both comprise the amino acid sequence of SEQ ID NO: 1596. In some embodiments, the first polypeptide and the second polypeptide both comprise the amino acid sequence of SEQ ID NO: 1615. In some embodiments, the first polypeptide and the second polypeptide both comprise the amino acid sequence of SEQ ID NO: 1626. In some embodiments, the first polypeptide and the second polypeptide both comprise the amino acid sequence of SEQ ID NO: 1818. In some embodiments, the first polypeptide and the second polypeptide both comprise the amino acid sequence of SEQ ID NO: 1822. In some embodiments, the first polypeptide and the second polypeptide both comprise the amino acid sequence of SEQ ID NO: 1825. In some embodiments, the first polypeptide and the second polypeptide both comprise the amino acid sequence of SEQ ID NO: 1826.

In some embodiments, the second polypeptide is conjugated to the antigen-binding protein (e.g., the anti-GIPR antibody) through a linker moiety, such as, e.g., a linker moiety described below, which may be the same or different from the linker moiety used to conjugate the first polypeptide to the antigen-binding protein (e.g., the anti-GIPR antibody). In some embodiments, the second polypeptide is conjugated to the antigen-binding protein (e.g., the anti-GIPR antibody) through a second linker polypeptide, such as, e.g., a linker polypeptide described below.

In some embodiments, the second linker polypeptide comprises the amino acid sequence of any one of SEQ ID NOs: 1628-1683, 1739-1746, or 1841-1852. In some embodiments, the second linker polypeptide comprises the amino acid sequence of any one of SEQ ID NOs: 1628-1683, 1739, 1850, or 1851. In some embodiments, the second linker polypeptide comprises the amino acid sequence of any one of SEQ ID NOs: 1740-1746, 1841-1849, or 1852.

In some embodiments, the second linker polypeptide comprises the amino acid sequence of any one of SEQ ID NOs: 1628-1683. In some embodiments, the second linker polypeptide comprises the amino acid sequence of any one of SEQ ID NOs: 1628-1630. In some embodiments, the second linker polypeptide comprises the amino acid sequence of SEQ ID NO: 1628. In some embodiments, the second linker polypeptide comprises the amino acid sequence of SEQ ID NO: 1629. In some embodiments, the second linker polypeptide comprises the amino acid sequence of SEQ ID NO: 1630.

In some embodiments, the first linker polypeptide and the second linker polypeptide each independently comprise the amino acid sequence of any one of SEQ ID NOs: 1628-1683, 1739-1746, or 1841-1852. In some embodiments, the first linker polypeptide and the second linker polypeptide each independently comprise the amino acid sequence of any one of SEQ ID NOs: 1628-1683, 1739, 1850, or 1851. In some embodiments, the first linker polypeptide and the second linker polypeptide each independently comprise the amino acid sequence of any one of SEQ ID NOs: 1740-1746, 1841-1849, or 1852.

In some embodiments, the first linker polypeptide and the second linker polypeptide each independently comprise the amino acid sequence of any one of SEQ ID NOs: 1628-1683. In some embodiments, the first linker polypeptide and the second linker polypeptide are identical and comprise the amino acid sequence of any one of SEQ ID NOs: 1628-1683.

In some embodiments, the first linker polypeptide and the second linker polypeptide each independently the amino acid sequence of any one of SEQ ID NOs: 1628-1630. In some embodiments, the first linker polypeptide and the second linker polypeptide are identical and comprise the amino acid sequence of any one of SEQ ID NOs: 1628-1630.

In some embodiments, the first linker polypeptide and the second linker polypeptide both comprise the amino acid sequence of any one of SEQ ID NOs: 1628-1683, 1739-1746, or 1841-1852. In some embodiments, the first linker polypeptide and the second linker polypeptide both comprise the amino acid sequence of any one of SEQ ID NOs: 1628-1683, 1739, 1850, or 1851. In some embodiments, the first linker polypeptide and the second linker polypeptide both comprise the amino acid sequence of any one of SEQ ID NOs: 1740-1746, 1841-1849, or 1852.

In some embodiments, the first linker polypeptide and the second linker polypeptide both comprise the amino acid sequence of SEQ ID NO: 1628. In some embodiments, the first linker polypeptide and the second linker polypeptide both comprise the amino acid sequence of SEQ ID NO: 1629. In some embodiments, the first linker polypeptide and the second linker polypeptide both comprise the amino acid sequence of SEQ ID NO: 1630.

In some embodiments, a C-terminus of the second polypeptide is covalently linked to a C-terminus of a second linker polypeptide. In some embodiments, the C-terminus of the second polypeptide is derivatized. In some embodiments, the C-terminus of the second linker polypeptide is derivatized. In some embodiments, the C-termini of both the second polypeptide and the second linker polypeptide are derivatized.

In some embodiments, a C-terminus of the first polypeptide is covalently linked to a C-terminus of a first linker polypeptide, and a C-terminus of the second polypeptide is covalently linked to a C-terminus of a second linker polypeptide.

In some embodiments, an ε-amino group of a lysine residue of the second polypeptide is covalently linked to a C-terminus of a second linker polypeptide. For example, in some embodiments, a lysine residue of the second polypeptide is covalently linked to a C-terminus of the second linker polypeptide via an amide bond, e.g., an amide bond formed by condensation of an ε-amino group of a lysine residue of the second polypeptide and a carboxyl group of a C-terminus of the second polypeptide linker.

In some embodiments, an β-amino group of a lysine residue of the first polypeptide is covalently linked to a C-terminus of a first linker polypeptide, and an ε-amino group of a lysine residue of the second polypeptide is covalently linked to a C-terminus of a second linker polypeptide. Illustratively, in some embodiments, a lysine residue of the first polypeptide is covalently linked to a C-terminus of the first linker polypeptide via an amide bond, e.g., an amide bond formed by condensation of an ε-amino group of a lysine residue of the first polypeptide and a carboxyl group of a C-terminus of the first polypeptide linker, and a lysine residue of the second polypeptide is covalently linked to a C-terminus of the second linker polypeptide via an amide bond, e.g., an amide bond formed by condensation of an ε-amino group of a lysine residue of the second polypeptide and a carboxyl group of a C-terminus of the second polypeptide linker.

In some embodiments, an N-terminus of the second linker polypeptide is conjugated to a cysteine residue of the antigen-binding protein (e.g., the anti-GIPR antibody). For example, in some embodiments, an derivatized N-terminus of the second linker polypeptide is covalently linked to a cysteine residue of the antigen-binding protein (e.g., the anti-GIPR antibody) via a thioether linkage that comprises a sulfur atom of the cysteine residue. In some embodiments, an acetylated N-terminus of the second linker polypeptide is covalently linked to a cysteine residue of the antigen-binding protein (e.g., the anti-GIPR antibody) via a thioether linkage that comprises a sulfur atom of the cysteine residue.

In some embodiments, the antigen-binding protein is an antibody and the cysteine residue of the antibody that is conjugated to the N-terminus of the second linker polypeptide is at a position selected from the group consisting of 88 of a light chain, 384 of a heavy chain, and 487 of a heavy chain, according to AHo numbering. In some embodiments, the cysteine residue of the antibody that is conjugated to the N-terminus of the second linker polypeptide is at position 384 of a heavy chain, according to AHo numbering.

In some embodiments, an ε-amino group of a lysine residue of the second polypeptide is covalently linked to a C-terminus of a second linker polypeptide, and an N-terminus of the second linker polypeptide is conjugated to a cysteine residue of the antigen-binding protein (e.g., the anti-GIPR antibody). Illustratively, in some embodiments, a lysine residue of the second polypeptide is covalently linked to a C-terminus of the second linker polypeptide via an amide bond, e.g., an amide bond formed by condensation of an ε-amino group of a lysine residue of the second polypeptide and a carboxyl group of a C-terminus of the second polypeptide linker, and a derivatized (e.g., acetylated) N-terminus of the second linker polypeptide is covalently linked to a cysteine residue of the antigen-binding protein (e.g., the anti-GIPR antibody) via a thioether linkage that comprises a sulfur atom of the cysteine residue.

In some embodiments, the antigen-binding protein is an antibody and the cysteine residue of the antibody that is conjugated to the N-terminus of the second linker polypeptide is at a position selected from the group consisting of 88 of a light chain, 384 of a heavy chain, and 487 of a heavy chain, according to AHo numbering. In some embodiments, the cysteine residue of the antibody that is conjugated to the N-terminus of the second linker polypeptide is at position 384 of a heavy chain, according to AHo numbering.

In some embodiments, the cysteine residues of the antibody that are conjugated to the N-termini of the first linker polypeptide and the second linker polypeptide are at positions selected from the group consisting of 88 of both light chains, 384 of both heavy chains, and 487 of both heavy chains, according to AHo numbering.

In some embodiments, the cysteine residues of the antibody that are conjugated to the N-termini of the first linker polypeptide and the second linker polypeptide are at positions 384 of both heavy chains, according to AHo numbering.

In some embodiments, a C-terminal amino acid residue of the second polypeptide is covalently linked to a N-terminal amino acid of a second linker polypeptide.

In some embodiments, a C-terminal amino acid residue of the first polypeptide is covalently linked to a N-terminal amino acid residue of a first linker polypeptide, and a C-terminal amino acid residue of the second polypeptide is covalently linked to a N-terminal amino acid of a second linker polypeptide.

In some embodiments, a C-terminus of the second linker polypeptide is conjugated to a cysteine residue of the antigen-binding protein (e.g., the anti-GIPR antibody). For example, in some embodiments, a derivatized C-terminus of the second linker polypeptide is covalently linked to a cysteine residue of the antigen-binding protein (e.g., the anti-GIPR antibody) via a thioether linkage that comprises a sulfur atom of the cysteine residue. In some embodiments, an acetylated C-terminus of the second linker polypeptide is covalently linked to a cysteine residue of the antigen-binding protein (e.g., the anti-GIPR antibody) via a thioether linkage that comprises a sulfur atom of the cysteine residue.

In some embodiments, the antigen-binding protein is an antibody and the cysteine residue of the antibody that is conjugated to the C-terminus of the second linker polypeptide is at a position selected from the group consisting of 88 of a light chain, 384 of a heavy chain, and 487 of a heavy chain, according to AHo numbering. In some embodiments, the cysteine residue of the antibody that is conjugated to the C-terminus of the second linker polypeptide is at position 384 of a heavy chain, according to AHo numbering.

In some embodiments, a C-terminal amino acid residue of the second polypeptide is covalently linked to a N-terminal amino acid residue of a second linker polypeptide, and a C-terminus of the second linker polypeptide is conjugated to a cysteine residue of the antigen-binding protein (e.g., the anti-GIPR antibody).

In some embodiments, the antigen-binding protein is an antibody and the cysteine residue of the antibody that is conjugated to the C-terminus of the second linker polypeptide is at a position selected from the group consisting of 88 of a light chain, 384 of a heavy chain, and 487 of a heavy chain, according to AHo numbering. In some embodiments, the cysteine residue of the antibody that is conjugated to the C-terminus of the second linker polypeptide is at position 384 of a heavy chain, according to AHo numbering.

In some embodiments, the cysteine residues of the antibody that are conjugated to the C-termini of the first linker polypeptide and the second linker polypeptide are at positions selected from the group consisting of 88 of both light chains, 384 of both heavy chains, and 487 of both heavy chains, according to AHo numbering.

In some embodiments, the cysteine residues of the antibody that are conjugated to the C-termini of the first linker polypeptide and the second linker polypeptide are at positions 384 of both heavy chains, according to AHo numbering.

Also provided herein is a molecule comprising:
a first polypeptide that agonizes a glucagon receptor (“GCGR”);
a first linker polypeptide;
an antibody that specifically binds to a glucose-dependent insulinotropic polypeptide receptor (“GIPR”);
a second linker polypeptide; and
a second polypeptide that agonizes a GCGR, wherein:
   an ε-amino group of a lysine residue of the first polypeptide is covalently linked to a C-terminus of the first linker polypeptide;
   an N-terminus of the first linker polypeptide is conjugated to a cysteine residue of the antibody;
   an ε-amino group of a lysine residue of the second polypeptide is covalently linked to a C-terminus of the second linker polypeptide; and
   an N-terminus of the second linker polypeptide is conjugated to a cysteine residue of the antibody.

In some embodiments, the antibody that specifically binds to a GIPR (i.e., the anti-GIPR antibody) can be any anti-GIPR antibody described herein (e.g., an anti-GIPR antibody of Table 1 or otherwise described herein).

In some embodiments, the anti-GIPR antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise the amino acid sequences of SEQ ID NO: 702, SEQ ID NO: 859, SEQ ID NO: 1016, SEQ ID NO: 1173, SEQ ID NO: 1330, and SEQ ID NO: 1487, respectively.

In some embodiments, the anti-GIPR antibody comprises a light chain variable region comprising the amino acid sequence of SEQ ID NO: 74 and a heavy chain variable region comprising the amino acid sequence of SEQ ID NO: 231.

In some embodiments, the anti-GIPR antibody comprises a light chain comprising the amino acid sequence of SEQ ID NO: 388 and a heavy chain comprising the amino acid sequence of SEQ ID NO: 1571.

In some embodiments, the first polypeptide and the second polypeptide can be any glucagon receptor agonist described herein (such as, e.g., glucagon (SEQ ID NO: 1576) or a glucagon analog (e.g., a variant of SEQ ID NO: 1576 that includes one or more amino modifications, which may include, but are not limited to, S2s, S16Aib, S16Q, S16E, R17K, R18A, R18Y, R18F, D21E, Q24D, Q24E, Q24K, W25-5-BrW, M27L, M27E, N28A, N28D, N28K, N28Q, and T29D modifications, as described above). In some embodiments, the second polypeptide has the same amino acid sequence as the first polypeptide. In some embodiments, the second polypeptide has a different amino acid sequence than the first polypeptide.

In some embodiments, the first polypeptide and the second polypeptide independently comprise amino acid sequences selected from SEQ ID NOs: 1587, 1592, 1615, 1626, 1818, 1822, 1825, and 1826. In some embodiments, the first polypeptide and the second polypeptide independently comprise amino acid sequences selected from SEQ ID NOs: 1626, 1818, 1822, 1825, and 1826.

In some embodiments, the first polypeptide and the second polypeptide independently comprise amino acid sequences selected from SEQ ID NOs: 1587, 1592, 1596, 1615, and 1626. In some embodiments, the first polypeptide and the second polypeptide independently comprise amino acid sequences selected from SEQ ID NOs: 1587, 1592, 1615, and 1626.

In some embodiments, the first polypeptide has the same amino acid sequence as the second polypeptide. In some embodiments, the first polypeptide has the same amino acid sequence as the second polypeptide, wherein the amino acid sequence is selected from SEQ ID NOs: 1587, 1592, 1615, 1626, 1818, 1822, 1825, and 1826. In some embodiments, the first polypeptide has the same amino acid sequence as the second polypeptide, wherein the amino acid sequence is selected from SEQ ID NOs: 1626, 1818, 1822, 1825, and 1826. In some embodiments, the first polypeptide has the same amino acid sequence as the second polypeptide, wherein the amino acid sequence is selected from SEQ ID NOs: 1587, 1592, 1596, 1615, and 1626. In some embodiments, the first polypeptide has the same amino acid sequence as the second polypeptide, wherein the amino acid sequence is selected from SEQ ID NOs: 1587, 1592, 1615, and 1626.

In some embodiments, the first linker polypeptide and the second linker polypeptide independently comprise amino acid sequences selected from SEQ ID NOs: 1628-1683, 1739-1746, and 1841-1852. In some embodiments, the first linker polypeptide and the second linker polypeptide independently comprise amino acid sequences selected from SEQ ID NOs: 1628-1683, 1739, 1850, and 1851. In some embodiments, the first linker polypeptide and the second linker polypeptide independently comprise amino acid sequences selected from SEQ ID NOs: 1740-1746, 1841-1849, and 1852.

In some embodiments, the first linker polypeptide and the second linker polypeptide independently comprise amino acid sequences selected from SEQ ID NOs: 1628-1683, preferably SEQ ID NOs: 1628-1630, preferably SEQ ID NO: 1630.

In some embodiments, the first linker polypeptide has the same amino acid sequence as the second linker polypeptide. In some embodiments, the first linker polypeptide has the same amino acid sequence as the second linker polypeptide, wherein the amino acid sequence is selected from 1628-1683, 1739-1746, and 1841-1852. In some embodiments, the first linker polypeptide has the same amino acid sequence as the second linker polypeptide, wherein the amino acid sequence is selected from SEQ ID NOs: 1628-1683, 1739, 1850, and 1851. In some embodiments, the first linker polypeptide has the same amino acid sequence as the second linker polypeptide, wherein the amino acid sequence is selected from 1740-1746, 1841-1849, and 1852. In some embodiments, the first linker polypeptide has the same amino acid sequence as the second linker polypeptide, wherein the amino acid sequence is selected from SEQ ID NOs: 1628-1683, preferably SEQ ID NOs: 1628-1630, preferably SEQ ID NO: 1630.

In some embodiments, the first polypeptide has the same amino acid sequence as the second polypeptide; and the first linker polypeptide has the same amino acid sequence as the second linker polypeptide. In some embodiments, the first polypeptide has the same amino acid sequence as the second polypeptide, wherein the amino acid sequence is selected from SEQ ID NOs: 1587-1627, 1747-1840, and 1859-1862, preferably SEQ ID NOs: 1587, 1592, 1615, 1626, 1818, 1822, 1825, or 1826, and the first linker polypeptide has the same amino acid sequence as the second linker polypeptide, wherein the amino acid sequence is selected from SEQ ID NOs: 1628-1683, 1739-1746, and 1841-1852, preferably SEQ ID NOs: 1628-1630, preferably SEQ ID NO: 1628 or SEQ ID NO: 1630.

In some embodiments, the first polypeptide has the same amino acid sequence as the second polypeptide, wherein the amino acid sequence is selected from SEQ ID NOs: 1587, 1592, 1596, 1615, and 1626, and the first linker polypeptide has the same amino acid sequence as the second linker polypeptide, wherein the amino acid sequence is selected from SEQ ID NOs: 1628-1683, preferably SEQ ID NOs: 1628-1630, preferably SEQ ID NO: 1630.

In some embodiments, the lysine residue of the first polypeptide comprising the ε-amino group that is covalently linked to the C-terminus of the first linker polypeptide is at position 21, position 24, position 28, or position 31. In some embodiments, the lysine residue of the first polypeptide comprising the ε-amino group that is covalently linked to the C-terminus of the first linker polypeptide is at position 24 or position 28. In some embodiments, the lysine residue is at position 21. In some embodiments, the lysine residue is at position 24. In some embodiments, the lysine residue is at position 28. In some embodiments, the lysine residue is at position 31.

In some embodiments, the lysine residue of the second polypeptide comprising the ε-amino group that is covalently linked to the C-terminus of the second linker polypeptide is at position 24 or position 28. In some embodiments, the lysine residue is at position 24. In some embodiments, the lysine residue is at position 28.

In some embodiments, the lysine residue of the first polypeptide comprising the ε-amino group that is covalently linked to the C-terminus of the first linker polypeptide is at position 24 or position 28; and the lysine residue of the second polypeptide comprising the ε-amino group that is covalently linked to the C-terminus of the second linker polypeptide is at position 24 or position 28.

In some embodiments, the lysine residue of the first polypeptide comprising the ε-amino group that is covalently linked to the C-terminus of the first linker polypeptide is at position 21; and the lysine residue of the second polypeptide comprising the ε-amino group that is covalently linked to the C-terminus of the second linker polypeptide is at position 21.

In some embodiments, the lysine residue of the first polypeptide comprising the ε-amino group that is covalently linked to the C-terminus of the first linker polypeptide is at position 24; and the lysine residue of the second polypeptide comprising the ε-amino group that is covalently linked to the C-terminus of the second linker polypeptide is at position 24.

In some embodiments, the lysine residue of the first polypeptide comprising the ε-amino group that is covalently linked to the C-terminus of the first linker polypeptide is at position 28; and the lysine residue of the second polypeptide comprising the ε-amino group that is covalently linked to the C-terminus of the second linker polypeptide is at position 28.

In some embodiments, the lysine residue of the first polypeptide comprising the ε-amino group that is covalently linked to the C-terminus of the first linker polypeptide is at position 31; and the lysine residue of the second polypeptide comprising the ε-amino group that is covalently linked to the C-terminus of the second linker polypeptide is at position 31.

In some embodiments, the cysteine residues of the antibody that are conjugated to the N-termini of the first linker polypeptide and the second linker polypeptide are at positions selected from the group consisting of 88 of both light chains, 384 of both heavy chains, and 487 of both heavy chains, according to AHo numbering.

In some embodiments, the cysteine residues of the antibody that are conjugated to the N-termini of the first linker polypeptide and the second linker polypeptide are at positions 384 of both heavy chains, according to AHo numbering.

In some embodiments, the lysine residue of the first polypeptide comprising the ε-amino group that is covalently linked to the C-terminus of the first linker polypeptide is at position 24; the lysine residue of the second polypeptide comprising the ε-amino group that is covalently linked to the C-terminus of the second linker polypeptide is at position 24; and the cysteine residues of the antibody that are conjugated to the N-termini of the first linker polypeptide and the second linker polypeptide are at positions 384 of both heavy chains, according to AHo numbering.

In some embodiments, the lysine residue of the first polypeptide comprising the ε-amino group that is covalently linked to the C-terminus of the first linker polypeptide is at position 28; the lysine residue of the second polypeptide comprising the ε-amino group that is covalently linked to the C-terminus of the second linker polypeptide is at position 28; and the cysteine residues of the antibody that are conjugated to the N-termini of the first linker polypeptide and the second linker polypeptide are at positions 384 of both heavy chains, according to AHo numbering.

Also provided herein is a molecule comprising:
a polypeptide that agonizes a glucagon receptor (“GCGR”) comprising the amino acid sequence of any one of SEQ ID NOs: 1587-1627, 1747-1749, 1751-1792, 1794-1798, 1801-1830, 1833-1840, 1859-1862, or 1879-1881;
a linker polypeptide comprising the amino acid sequence of any one of SEQ ID NOs: 1628-1683, 1739-1746, or 1841-1852 (e.g., SEQ ID NOs: 1628-1683, 1739, 1850, or 1851; SEQ ID NOs: 1628-1630); and
an antibody that specifically binds to a glucose-dependent insulinotropic polypeptide receptor (“GIPR”), wherein the antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise the amino acid sequences of SEQ ID NO: 702, SEQ ID NO: 859, SEQ ID NO: 1016, SEQ ID NO: 1173, SEQ ID NO: 1330, and SEQ ID NO: 1487, respectively,
wherein:
   an ε-amino group of a lysine residue at position 21, position 24, position 28, or position 31 of the polypeptide is covalently linked to a C-terminus of the linker polypeptide; and
   an N-terminus of the linker polypeptide is conjugated to a cysteine residue of the antibody.

In some embodiments, the linker polypeptide comprises an amino acid sequence selected from SEQ ID NOs: 1628-1683, 1739, 1850, and 1851.

In some embodiments, the polypeptide comprises the amino acid sequence of any one of SEQ ID NOs: 1587-1627, 1747-1749, 1751-1788, 1790-1792, 1794-1798, 1801-1830, 1833-1840, 1859, 1861, or 1862, and an ε-amino group of a lysine residue at position 24 or position 28 of the polypeptide is covalently linked to a C-terminus of the linker polypeptide.

In some embodiments, the polypeptide comprises the amino acid sequence of any one of SEQ ID NOs: 1589, 1592, 1594, 1597, 1598, 1613, 1625, 1762, 1766-1768, 1787, 1788, 1797, 1798, 1804, 1805, 1811, 1812, 1821, 1822, 1833, 1837, or 1838, and an ε-amino group of a lysine residue at position 24 of the polypeptide is covalently linked to a C-terminus of the linker polypeptide.

In some embodiments, the polypeptide comprises the amino acid sequence of any one of SEQ ID NOs: 1587, 1588, 1590, 1591, 1593, 1595, 1596, 1599-1612, 1614-1624, 1626, 1627, 1747-1749, 1751-1761, 1763-1765, 1769-1786, 1790-1792, 1794-1796, 1801-1803, 1806-1810, 1813-1820, 1823-1830, 1834-1836, 1839, 1840, 1859, 1861, or 1862, and an ε-amino group of a lysine residue at position 28 of the polypeptide is covalently linked to a C-terminus of the linker polypeptide.

In some embodiments, the antibody comprises a light chain variable region comprising the amino acid sequence of SEQ ID NO: 74 and a heavy chain variable region comprising the amino acid sequence of SEQ ID NO: 231.

In some embodiments, an N-terminus of the linker polypeptide is conjugated to a cysteine residue of the antibody at a position selected from 88 of a light chain, 384 of a heavy chain, and 487 of a heavy chain, according to AHo numbering.

In some embodiments, an N-terminus of the linker polypeptide is conjugated to a cysteine residue at position 275 of the heavy chain of the antibody.

In some embodiments, the antibody comprises a light chain and a heavy chain, wherein the light chain comprises the amino acid sequence of SEQ ID NO: 388 and the heavy chain comprises the amino acid sequence of SEQ ID NO: 1571.

In some embodiments, the antibody comprises a light chain and a heavy chain, wherein the light chain comprises the amino acid sequence of SEQ ID NO: 388 and the heavy chain comprises the amino acid sequence of SEQ ID NO: 1571, and an N-terminus of the linker polypeptide is conjugated to a cysteine residue at position 275 of the heavy chain of the antibody.

Also provided herein is a molecule comprising:
a polypeptide that agonizes a glucagon receptor (“GCGR”) comprising the amino acid sequence of any one of SEQ ID NOs: 1587-1627 or 1747;
a linker polypeptide comprising the amino acid sequence of any one of SEQ ID NOs: 1628-1683 (e.g., SEQ ID NOs: 1628-1630); and
an antibody that specifically binds to a glucose-dependent insulinotropic polypeptide receptor (“GIPR”), wherein the antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise the amino acid sequences of SEQ ID NO: 702, SEQ ID NO: 859, SEQ ID NO: 1016, SEQ ID NO: 1173, SEQ ID NO: 1330, and SEQ ID NO: 1487, respectively,
wherein:
   an ε-amino group of a lysine residue at position 24 or position 28 of the polypeptide is covalently linked to a C-terminus of the linker polypeptide; and
   an N-terminus of the linker polypeptide is conjugated to a cysteine residue of the antibody.

In some embodiments, the antibody comprises a light chain variable region comprising the amino acid sequence of SEQ ID NO: 74 and a heavy chain variable region comprising the amino acid sequence of SEQ ID NO: 231.

In some embodiments, an N-terminus of the linker polypeptide is conjugated to a cysteine residue of the antibody at a position selected from 88 of a light chain, 384 of a heavy chain, and 487 of a heavy chain, according to AHo numbering.

In some embodiments, an N-terminus of the linker polypeptide is conjugated to a cysteine residue at position 275 of the heavy chain of the antibody.

In some embodiments, the antibody comprises a light chain and a heavy chain, wherein the light chain comprises the amino acid sequence of SEQ ID NO: 388 and the heavy chain comprises the amino acid sequence of SEQ ID NO: 1571.

In some embodiments, the antibody comprises a light chain and a heavy chain, wherein the light chain comprises the amino acid sequence of SEQ ID NO: 388 and the heavy chain comprises the amino acid sequence of SEQ ID NO: 1571, and an N-terminus of the linker polypeptide is conjugated to a cysteine residue at position 275 of the heavy chain of the antibody.

Also provided herein is a molecule comprising:
a polypeptide that agonizes a glucagon receptor (“GCGR”) comprising the amino acid sequence of any one of SEQ ID NOs: 1587-1627 (e.g., SEQ ID NOs: 1587, 1592, 1596, 1615, or 1626);
a linker polypeptide comprising the amino acid sequence of any one of SEQ ID NOs: 1628-1683 (e.g., SEQ ID NOs: 1628-1630); and
an antibody that specifically binds to a glucose-dependent insulinotropic polypeptide receptor (“GIPR”), wherein the antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise the amino acid sequences of SEQ ID NO: 702, SEQ ID NO: 859, SEQ ID NO: 1016, SEQ ID NO: 1173, SEQ ID NO: 1330, and SEQ ID NO: 1487, respectively,
wherein:
   an ε-amino group of a lysine residue at position 24 or position 28 of the polypeptide is covalently linked to a C-terminus of the linker polypeptide; and
   an N-terminus of the linker polypeptide is conjugated to a cysteine residue of the antibody.

In some embodiments, the antibody comprises a light chain variable region comprising the amino acid sequence of SEQ ID NO: 74 and a heavy chain variable region comprising the amino acid sequence of SEQ ID NO: 231.

In some embodiments, an N-terminus of the linker polypeptide is conjugated to a cysteine residue of the antibody at a position selected from 88 of a light chain, 384 of a heavy chain, and 487 of a heavy chain, according to AHo numbering.

In some embodiments, an N-terminus of the linker polypeptide is conjugated to a cysteine residue at position 275 of the heavy chain of the antibody.

In some embodiments, the antibody comprises a light chain and a heavy chain, wherein the light chain comprises the amino acid sequence of SEQ ID NO: 388 and the heavy chain comprises the amino acid sequence of SEQ ID NO: 1571.

In some embodiments, the antibody comprises a light chain and a heavy chain, wherein the light chain comprises the amino acid sequence of SEQ ID NO: 388 and the heavy chain comprises the amino acid sequence of SEQ ID NO: 1571, and an N-terminus of the linker polypeptide is conjugated to a cysteine residue at position 275 of the heavy chain of the antibody.

Also provided herein is a molecule comprising:
a first polypeptide and a second polypeptide that agonize a glucagon receptor (“GCGR”), wherein each of the first polypeptide and the second polypeptide comprise an amino acid sequence independently selected from SEQ ID NOs: 1587-1627, 1747-1749, 1751-1792, 1794-1798, 1801-1830, 1833-1840, and 1859-1862;
a first linker polypeptide and a second linker polypeptide, wherein each of the first linker polypeptide and the second linker polypeptide comprise an amino acid sequence selected from SEQ ID NOs: 1628-1683, 1739-1746, and 1841-1852 (e.g., SEQ ID NOs: 1628-1683; SEQ ID NOs: 1628-1630); and
an antibody that specifically binds to a glucose-dependent insulinotropic polypeptide receptor (“GIPR”), wherein the antibody comprises a first light chain and a second light chain, each comprising a CDRL1, a CDRL2, and a CDRL3, wherein the CDRL1, the CDRL2, and the CDRL3 comprise the amino acid sequences of SEQ ID NO: 702, SEQ ID NO: 859, and SEQ ID NO: 1016, respectively, and a first heavy chain and a second heavy chain, each comprising a CDRH1, a CDRH2, and a CDRH3, wherein the CDRH1, the CDRH2, and the CDRH3 comprise SEQ ID NO: 1173, SEQ ID NO: 1330, and SEQ ID NO: 1487, respectively,
wherein:
   an ε-amino group of a lysine residue at position 21, position 24, position 28, or position 31 of the first polypeptide is covalently linked to a C-terminus of the first linker polypeptide;
   an N-terminus of the first linker polypeptide is conjugated to a cysteine residue of the first heavy chain of the antibody;
   an ε-amino group of a lysine residue at position 21, position 24, position 28, or position 31 of the second polypeptide is covalently linked to a C-terminus of the second linker polypeptide; and
   an N-terminus of the second linker polypeptide is conjugated to a cysteine residue of the second heavy chain of the antibody.

In some embodiments, each of the first linker polypeptide and the second linker polypeptide comprise an amino acid sequence selected from SEQ ID NOs: 1628-1683, 1739, 1850, and 1851.

In some embodiments, each of the first polypeptide and the second polypeptide comprise an amino acid sequence independently selected from SEQ ID NOs: 1589, 1592, 1594, 1597, 1598, 1613, 1625, 1762, 1766-1768, 1787, 1788, 1797, 1798, 1804, 1805, 1811, 1812, 1821, 1822, 1833, 1837, and 1838, the ε-amino group of a lysine residue at position 24 of the first polypeptide is covalently linked to a C-terminus of the first linker polypeptide, and the ε-amino group of a lysine residue at position 24 of the second polypeptide is covalently linked to a C-terminus of the second linker polypeptide.

In some embodiments, each of the first polypeptide and the second polypeptide comprise an amino acid sequence independently selected from SEQ ID NOs: 1587, 1588, 1590, 1591, 1593, 1595, 1596, 1599-1612, 1614-1624, 1626, 1627, 1747-1749, 1751-1761, 1763-1765, 1769-1786, 1790-1792, 1794-1796, 1801-1803, 1806-1810, 1813-1820, 1823-1830, 1834-1836, 1839, 1840, 1859, 1861, and 1862, the ε-amino group of a lysine residue at position 28 of the first polypeptide is covalently linked to a C-terminus of the first linker polypeptide, and the ε-amino group of a lysine residue at position 28 of the second polypeptide is covalently linked to a C-terminus of the second linker polypeptide.

In some embodiments, the first light chain and the second light chain each comprise a light chain variable region comprising the amino acid sequence of SEQ ID NO: 74.

In some embodiments, the first heavy chain and the second heavy chain each comprise a heavy chain variable region comprising the amino acid sequence of SEQ ID NO: 231.

In some embodiments, the first light chain and the second light chain each comprise a light chain variable region comprising the amino acid sequence of SEQ ID NO: 74, and the first heavy chain and the second heavy chain each comprise a heavy chain variable region comprising the amino acid sequence of SEQ ID NO: 231.

In some embodiments, an N-terminus of the first linker polypeptide is conjugated to a cysteine residue of the antibody at a position selected from 88 of the first light chain, 384 of the first heavy chain, and 487 of the first heavy chain, according to AHo numbering.

In some embodiments, an N-terminus of the second linker polypeptide is conjugated to a cysteine residue of the antibody at a position selected from 88 of the second light chain, 384 of the second heavy chain, and 487 of the second heavy chain, according to AHo numbering.

In some embodiments, an N-terminus of the first linker polypeptide is conjugated to a cysteine residue of the antibody at a position selected from 88 of the first light chain, 384 of the first heavy chain, and 487 of the first heavy chain, according to AHo numbering, and an N-terminus of the second linker polypeptide is conjugated to a cysteine residue of the antibody at a position selected from 88 of the second light chain, 384 of the second heavy chain, and 487 of the second heavy chain, according to AHo numbering.

In some embodiments, the cysteine residues of the antibody that are conjugated to the N-termini of the first linker polypeptide and the second linker polypeptide are at positions 384 of the first heavy chain and the second heavy chain, respectively, according to AHo numbering.

In some embodiments, the first light chain and the second light chain each comprise the amino acid sequence of SEQ ID NO: 388.

In some embodiments, the first heavy chain and the second heavy chain each comprise the amino acid sequence of SEQ ID NO: 1571.

In some embodiments, the first light chain and the second light chain each comprise the amino acid sequence of SEQ ID NO: 388, and the first heavy chain and the second heavy chain each comprise the amino acid sequence of SEQ ID NO: 1571.

In some embodiments, an N-terminus of the first linker polypeptide is conjugated to a cysteine residue at position 275 of the first heavy chain of the antibody.

In some embodiments, an N-terminus of the second linker polypeptide is conjugated to a cysteine residue at position 275 of the second heavy chain of the antibody.

In some embodiments, an N-terminus of the first linker polypeptide is conjugated to a cysteine residue at position 275 of the first heavy chain of the antibody, and an N-terminus of the second linker polypeptide is conjugated to a cysteine residue at position 275 of the second heavy chain of the antibody.

In some embodiments, the first light chain and the second light chain each comprise the amino acid sequence of SEQ ID NO: 388, the first heavy chain and the second heavy chain each comprise the amino acid sequence of SEQ ID NO: 1571, an N-terminus of the first linker polypeptide is conjugated to a cysteine residue at position 275 of the first heavy chain of the antibody, and an N-terminus of the second linker polypeptide is conjugated to a cysteine residue at position 275 of the second heavy chain of the antibody.

In some embodiments, the first polypeptide and the second polypeptide have identical amino acid sequences. In some embodiments, the first linker polypeptide and the second linker polypeptide have identical amino acid sequences. In some embodiments, the first polypeptide and the second polypeptide have identical amino acid sequences, and the first linker polypeptide and the second linker polypeptide have identical amino acid sequences.

Also provided herein is a molecule comprising:
a first polypeptide and a second polypeptide that agonize a glucagon receptor (“GCGR”), wherein each of the first polypeptide and the second polypeptide comprise an amino acid sequence independently selected from SEQ ID NOs: 1587-1627 or 1747;
a first linker polypeptide and a second linker polypeptide, wherein each of the first linker polypeptide and the second linker polypeptide comprise an amino acid sequence selected from SEQ ID NOs: 1628-1683 (e.g., SEQ ID NOs: 1628-1630); and
an antibody that specifically binds to a glucose-dependent insulinotropic polypeptide receptor (“GIPR”), wherein the antibody comprises a first light chain and a second light chain, each comprising a CDRL1, a CDRL2, and a CDRL3, wherein the CDRL1, the CDRL2, and the CDRL3 comprise the amino acid sequences of SEQ ID NO: 702, SEQ ID NO: 859, and SEQ ID NO: 1016, respectively, and a first heavy chain and a second heavy chain, each comprising a CDRH1, a CDRH2, and a CDRH3, wherein the CDRH1, the CDRH2, and the CDRH3 comprise SEQ ID NO: 1173, SEQ ID NO: 1330, and SEQ ID NO: 1487, respectively,
wherein:
   an ε-amino group of a lysine residue at position 24 or position 28 of the first polypeptide is covalently linked to a C-terminus of the first linker polypeptide;
   an N-terminus of the first linker polypeptide is conjugated to a cysteine residue of the first heavy chain of the antibody;
   an ε-amino group of a lysine residue at position 24 or position 28 of the second polypeptide is covalently linked to a C-terminus of the second linker polypeptide; and
   an N-terminus of the second linker polypeptide is conjugated to a cysteine residue of the second heavy chain of the antibody.

In some embodiments, the first light chain and the second light chain each comprise a light chain variable region comprising the amino acid sequence of SEQ ID NO: 74.

In some embodiments, the first heavy chain and the second heavy chain each comprise a heavy chain variable region comprising the amino acid sequence of SEQ ID NO: 231.

In some embodiments, the first light chain and the second light chain each comprise a light chain variable region comprising the amino acid sequence of SEQ ID NO: 74, and the first heavy chain and the second heavy chain each comprise a heavy chain variable region comprising the amino acid sequence of SEQ ID NO: 231.

In some embodiments, an N-terminus of the first linker polypeptide is conjugated to a cysteine residue of the antibody at a position selected from 88 of the first light chain, 384 of the first heavy chain, and 487 of the first heavy chain, according to AHo numbering.

In some embodiments, an N-terminus of the second linker polypeptide is conjugated to a cysteine residue of the antibody at a position selected from 88 of the second light chain, 384 of the second heavy chain, and 487 of the second heavy chain, according to AHo numbering.

In some embodiments, an N-terminus of the first linker polypeptide is conjugated to a cysteine residue of the antibody at a position selected from 88 of the first light chain, 384 of the first heavy chain, and 487 of the first heavy chain, according to AHo numbering, and an N-terminus of the second linker polypeptide is conjugated to a cysteine residue of the antibody at a position selected from 88 of the second light chain, 384 of the second heavy chain, and 487 of the second heavy chain, according to AHo numbering.

In some embodiments, the cysteine residues of the antibody that are conjugated to the N-termini of the first linker polypeptide and the second linker polypeptide are at positions 384 of the first heavy chain and the second heavy chain, respectively, according to AHo numbering.

In some embodiments, the first light chain and the second light chain each comprise the amino acid sequence of SEQ ID NO: 388.

In some embodiments, the first heavy chain and the second heavy chain each comprise the amino acid sequence of SEQ ID NO: 1571.

In some embodiments, the first light chain and the second light chain each comprise the amino acid sequence of SEQ ID NO: 388, and the first heavy chain and the second heavy chain each comprise the amino acid sequence of SEQ ID NO: 1571.

In some embodiments, an N-terminus of the first linker polypeptide is conjugated to a cysteine residue at position 275 of the first heavy chain of the antibody.

In some embodiments, an N-terminus of the second linker polypeptide is conjugated to a cysteine residue at position 275 of the second heavy chain of the antibody.

In some embodiments, an N-terminus of the first linker polypeptide is conjugated to a cysteine residue at position 275 of the first heavy chain of the antibody, and an N-terminus of the second linker polypeptide is conjugated to a cysteine residue at position 275 of the second heavy chain of the antibody.

In some embodiments, the first light chain and the second light chain each comprise the amino acid sequence of SEQ ID NO: 388, the first heavy chain and the second heavy chain each comprise the amino acid sequence of SEQ ID NO: 1571, an N-terminus of the first linker polypeptide is conjugated to a cysteine residue at position 275 of the first heavy chain of the antibody, and an N-terminus of the second linker polypeptide is conjugated to a cysteine residue at position 275 of the second heavy chain of the antibody.

In some embodiments, the first polypeptide and the second polypeptide have identical amino acid sequences. In some embodiments, the first linker polypeptide and the second linker polypeptide have identical amino acid sequences. In some embodiments, the first polypeptide and the second polypeptide have identical amino acid sequences, and the first linker polypeptide and the second linker polypeptide have identical amino acid sequences.

Also provided herein is a molecule comprising:
a first polypeptide and a second polypeptide that agonize a glucagon receptor (“GCGR”), wherein each of the first polypeptide and the second polypeptide comprise an amino acid sequence independently selected from SEQ ID NOs: 1587-1627 (e.g., SEQ ID NOs: 1587, 1592, 1596, 1615, or 1626);
a first linker polypeptide and a second linker polypeptide, wherein each of the first linker polypeptide and the second linker polypeptide comprise an amino acid sequence selected from SEQ ID NOs: 1628-1683 (e.g., SEQ ID NOs: 1628-1630); and
an antibody that specifically binds to a glucose-dependent insulinotropic polypeptide receptor (“GIPR”), wherein the antibody comprises a first light chain and a second light chain, each comprising a CDRL1, a CDRL2, and a CDRL3, wherein the CDRL1, the CDRL2, and the CDRL3 comprise the amino acid sequences of SEQ ID NO: 702, SEQ ID NO: 859, and SEQ ID NO: 1016, respectively, and a first heavy chain and a second heavy chain, each comprising a CDRH1, a CDRH2, and a CDRH3, wherein the CDRH1, the CDRH2, and the CDRH3 comprise SEQ ID NO: 1173, SEQ ID NO: 1330, and SEQ ID NO: 1487, respectively,
wherein:
   an ε-amino group of a lysine residue at position 24 or position 28 of the first polypeptide is covalently linked to a C-terminus of the first linker polypeptide;
   an N-terminus of the first linker polypeptide is conjugated to a cysteine residue of the first heavy chain of the antibody;
   an ε-amino group of a lysine residue at position 24 or position 28 of the second polypeptide is covalently linked to a C-terminus of the second linker polypeptide; and
   an N-terminus of the second linker polypeptide is conjugated to a cysteine residue of the second heavy chain of the antibody.

In some embodiments, the first light chain and the second light chain each comprise a light chain variable region comprising the amino acid sequence of SEQ ID NO: 74.

In some embodiments, the first heavy chain and the second heavy chain each comprise a heavy chain variable region comprising the amino acid sequence of SEQ ID NO: 231.

In some embodiments, the first light chain and the second light chain each comprise a light chain variable region comprising the amino acid sequence of SEQ ID NO: 74, and the first heavy chain and the second heavy chain each comprise a heavy chain variable region comprising the amino acid sequence of SEQ ID NO: 231.

In some embodiments, an N-terminus of the first linker polypeptide is conjugated to a cysteine residue of the antibody at a position selected from 88 of the first light chain, 384 of the first heavy chain, and 487 of the first heavy chain, according to AHo numbering.

In some embodiments, an N-terminus of the second linker polypeptide is conjugated to a cysteine residue of the antibody at a position selected from 88 of the second light chain, 384 of the second heavy chain, and 487 of the second heavy chain, according to AHo numbering.

In some embodiments, an N-terminus of the first linker polypeptide is conjugated to a cysteine residue of the antibody at a position selected from 88 of the first light chain, 384 of the first heavy chain, and 487 of the first heavy chain, according to AHo numbering, and an N-terminus of the second linker polypeptide is conjugated to a cysteine residue of the antibody at a position selected from 88 of the second light chain, 384 of the second heavy chain, and 487 of the second heavy chain, according to AHo numbering.

In some embodiments, the cysteine residues of the antibody that are conjugated to the N-termini of the first linker polypeptide and the second linker polypeptide are at positions 384 of the first heavy chain and the second heavy chain, respectively, according to AHo numbering.

In some embodiments, the first light chain and the second light chain each comprise the amino acid sequence of SEQ ID NO: 388.

In some embodiments, the first heavy chain and the second heavy chain each comprise the amino acid sequence of SEQ ID NO: 1571.

In some embodiments, the first light chain and the second light chain each comprise the amino acid sequence of SEQ ID NO: 388, and the first heavy chain and the second heavy chain each comprise the amino acid sequence of SEQ ID NO: 1571.

In some embodiments, an N-terminus of the first linker polypeptide is conjugated to a cysteine residue at position 275 of the first heavy chain of the antibody.

In some embodiments, an N-terminus of the second linker polypeptide is conjugated to a cysteine residue at position 275 of the second heavy chain of the antibody.

In some embodiments, an N-terminus of the first linker polypeptide is conjugated to a cysteine residue at position 275 of the first heavy chain of the antibody, and an N-terminus of the second linker polypeptide is conjugated to a cysteine residue at position 275 of the second heavy chain of the antibody.

In some embodiments, the first light chain and the second light chain each comprise the amino acid sequence of SEQ ID NO: 388, the first heavy chain and the second heavy chain each comprise the amino acid sequence of SEQ ID NO: 1571, an N-terminus of the first linker polypeptide is conjugated to a cysteine residue at position 275 of the first heavy chain of the antibody, and an N-terminus of the second linker polypeptide is conjugated to a cysteine residue at position 275 of the second heavy chain of the antibody.

In some embodiments, the first polypeptide and the second polypeptide have identical amino acid sequences. In some embodiments, the first linker polypeptide and the second linker polypeptide have identical amino acid sequences. In some embodiments, the first polypeptide and the second polypeptide have identical amino acid sequences, and the first linker polypeptide and the second linker polypeptide have identical amino acid sequences.

Also provided herein is a molecule comprising:
a polypeptide that agonizes a glucagon receptor (“GCGR”) comprising the amino acid sequence of SEQ ID NO: 1615; a linker polypeptide comprising the amino acid sequence of SEQ ID NO: 1629; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Also provided herein is a molecule comprising:
a polypeptide that agonizes a glucagon receptor (“GCGR”) comprising the amino acid sequence of SEQ ID NO: 1626;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Also provided herein is a molecule comprising:
a polypeptide that agonizes a glucagon receptor (“GCGR”) comprising the amino acid sequence of SEQ ID NO: 1592;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 24 of the polypeptide is covalently linked to a C-terminus of the linker polypeptide; and
   an N-terminus of the linker polypeptide is conjugated to a cysteine residue at position 275 of the heavy chain of the antibody.

Also provided herein is a molecule comprising:
a polypeptide that agonizes a glucagon receptor (“GCGR”) comprising the amino acid sequence of SEQ ID NO: 1626;
a linker polypeptide comprising the amino acid sequence of SEQ ID NO: 1629; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Also provided herein is a molecule comprising:
a polypeptide that agonizes a glucagon receptor (“GCGR”) comprising the amino acid sequence of SEQ ID NO: 1587;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Also provided herein is a molecule comprising:
a polypeptide that agonizes a glucagon receptor (“GCGR”) comprising the amino acid sequence of SEQ ID NO: 1587;
a linker polypeptide comprising the amino acid sequence of SEQ ID NO: 1630; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Also provided herein is a molecule comprising:
a polypeptide that agonizes a glucagon receptor (“GCGR”) comprising the amino acid sequence of SEQ ID NO: 1822;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 24 of the polypeptide is covalently linked to a C-terminus of the linker polypeptide; and
   an N-terminus of the linker polypeptide is conjugated to a cysteine residue at position 275 of the heavy chain of the antibody.

Also provided herein is a molecule comprising:
a polypeptide that agonizes a glucagon receptor (“GCGR”) comprising the amino acid sequence of SEQ ID NO: 1825;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Also provided herein is a molecule comprising:
a polypeptide that agonizes a glucagon receptor (“GCGR”) comprising the amino acid sequence of SEQ ID NO: 1826;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Also provided herein is a molecule comprising:
a polypeptide that agonizes a glucagon receptor (“GCGR”) comprising the amino acid sequence of SEQ ID NO: 1818;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

Also provided herein is a molecule comprising:
a first polypeptide and a second polypeptide that agonize a glucagon receptor (“GCGR”), wherein each of the first polypeptide and the second polypeptide comprise an amino acid sequence independently selected from SEQ ID NOs: 1587-1627 or 1747; a first linker polypeptide and a second linker polypeptide, wherein each of the first linker polypeptide and the second linker polypeptide comprise an amino acid sequence selected from SEQ ID NOs: 1628-1683 (e.g., SEQ ID NOs: 1628-1630); and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 24 or position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 24 or position 28 of the second polypeptide is covalently linked to a C-terminus of the second linker polypeptide; and
   an N-terminus of the second linker polypeptide is conjugated to a cysteine residue at position 275 of the second heavy chain of the antibody.

In some embodiments, the first polypeptide and the second polypeptide have identical amino acid sequences. In some embodiments, the first linker polypeptide and the second linker polypeptide have identical amino acid sequences. In some embodiments, the first polypeptide and the second polypeptide have identical amino acid sequences, and the first linker polypeptide and the second linker polypeptide have identical amino acid sequences.

Also provided herein is a molecule comprising:
a first polypeptide and a second polypeptide that agonize a glucagon receptor (“GCGR”), wherein each of the first polypeptide and the second polypeptide comprise an amino acid sequence independently selected from SEQ ID NOs: 1587-1627, 1747-1749, 1751-1792, 1794-1798, 1801-1830, 1833-1840, and 1859-1862;
a first linker polypeptide and a second linker polypeptide, wherein each of the first linker polypeptide and the second linker polypeptide comprise an amino acid sequence selected from SEQ ID NOs: 1628-1683, 1739-1746, and 1841-1852; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 21, position 24, position 28, or position 31 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 21, position 24, position 28, or position 31 of the second polypeptide is covalently linked to a C-terminus of the second linker polypeptide; and
   an N-terminus of the second linker polypeptide is conjugated to a cysteine residue at position 275 of the second heavy chain of the antibody.

In some embodiments, each of the first linker polypeptide and the second linker polypeptide comprise an amino acid sequence selected from SEQ ID NOs: 1628-1683, 1739, 1850, and 1851.

In some embodiments, the first polypeptide and the second polypeptide have identical amino acid sequences. In some embodiments, the first linker polypeptide and the second linker polypeptide have identical amino acid sequences. In some embodiments, the first polypeptide and the second polypeptide have identical amino acid sequences, and the first linker polypeptide and the second linker polypeptide have identical amino acid sequences.

In some embodiments, the first polypeptide and the second polypeptide comprise an amino acid sequence independently selected from SEQ ID NOs: 1589, 1592, 1594, 1597, 1598, 1613, 1625, 1762, 1766-1768, 1787, 1788, 1797, 1798, 1804, 1805, 1811, 1812, 1821, 1822, 1833, 1837, and 1838, the ε-amino group of the lysine residue at position 24 of the first polypeptide is covalently linked to the C-terminus of the first linker polypeptide, and the ε-amino group of the lysine residue at position 24 of the second polypeptide is covalently linked to the C-terminus of the second linker polypeptide. In some embodiments, the first polypeptide and the second polypeptide have identical amino acid sequences. In some embodiments, the first linker polypeptide and the second linker polypeptide have identical amino acid sequences. In some embodiments, the first polypeptide and the second polypeptide have identical amino acid sequences, and the first linker polypeptide and the second linker polypeptide have identical amino acid sequences.

In some embodiments, the first polypeptide and the second polypeptide comprise an amino acid sequence independently selected from SEQ ID NOs: 1587, 1588, 1590, 1591, 1593, 1595, 1596, 1599-1612, 1614-1624, 1626, 1627, 1747-1749, 1751-1761, 1763-1765, 1769-1786, 1790-1792, 1794-1796, 1801-1803, 1806-1810, 1813-1820, 1823-1830, 1834-1836, 1839, 1840, 1859, 1861, and 1862, the ε-amino group of the lysine residue at position 28 of the first polypeptide is covalently linked to the C-terminus of the first linker polypeptide, and the ε-amino group of the lysine residue at position 28 of the second polypeptide is covalently linked to the C-terminus of the second linker polypeptide. In some embodiments, the first polypeptide and the second polypeptide have identical amino acid sequences. In some embodiments, the first linker polypeptide and the second linker polypeptide have identical amino acid sequences. In some embodiments, the first polypeptide and the second polypeptide have identical amino acid sequences, and the first linker polypeptide and the second linker polypeptide have identical amino acid sequences.

In some embodiments, the first polypeptide and the second polypeptide comprise an amino acid sequence of SEQ ID NO: 1860, the ε-amino group of the lysine residue at position 21 of the first polypeptide is covalently linked to the C-terminus of the first linker polypeptide, and the ε-amino group of the lysine residue at position 21 of the second polypeptide is covalently linked to the C-terminus of the second linker polypeptide. In some embodiments, the first linker polypeptide and the second linker polypeptide have identical amino acid sequences.

In some embodiments, the first polypeptide and the second polypeptide comprise an amino acid sequence of SEQ ID NO: 1789, the ε-amino group of the lysine residue at position 31 of the first polypeptide is covalently linked to the C-terminus of the first linker polypeptide, and the ε-amino group of the lysine residue at position 31 of the second polypeptide is covalently linked to the C-terminus of the second linker polypeptide. In some embodiments, the first linker polypeptide and the second linker polypeptide have identical amino acid sequences.

Also provided herein is a molecule comprising:
a first polypeptide and a second polypeptide that agonize a glucagon receptor (“GCGR”), wherein each of the first polypeptide and the second polypeptide comprise an amino acid sequence independently selected from SEQ ID NOs: 1587-1627 (e.g., SEQ ID NOs: 1587, 1592, 1596, 1615, or 1626);
a first linker polypeptide and a second linker polypeptide, wherein each of the first linker polypeptide and the second linker polypeptide comprise an amino acid sequence selected from SEQ ID NOs: 1628-1683 (e.g., SEQ ID NOs: 1628-1630); and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 24 or position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 24 or position 28 of the second polypeptide is covalently linked to a C-terminus of the second linker polypeptide; and
   an N-terminus of the second linker polypeptide is conjugated to a cysteine residue at position 275 of the second heavy chain of the antibody.

In some embodiments, the first polypeptide and the second polypeptide have identical amino acid sequences. In some embodiments, the first linker polypeptide and the second linker polypeptide have identical amino acid sequences. In some embodiments, the first polypeptide and the second polypeptide have identical amino acid sequences, and the first linker polypeptide and the second linker polypeptide have identical amino acid sequences.

Also provided herein is a molecule comprising:
a first polypeptide and a second polypeptide that agonize a glucagon receptor (“GCGR”), wherein each of the first polypeptide and the second polypeptide comprise an amino acid sequence independently selected from SEQ ID NOs: 1587-1627, 1747-1840, and 1859-1862;
a first linker polypeptide and a second linker polypeptide, wherein each of the first linker polypeptide and the second linker polypeptide comprise an amino acid sequence selected from SEQ ID NOs: 1628-1683, 1739-1746, and 1841-1852; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   a C-terminal amino acid residue of the first polypeptide is covalently linked to an N-terminal amino acid residue of the first linker polypeptide;
   a C-terminal amino acid residue of the first linker polypeptide is conjugated to a cysteine residue at position 275 of the first heavy chain of the antibody;
   a C-terminal amino acid residue of the second polypeptide is covalently linked to an N-terminal amino acid residue of the second linker polypeptide; and
   a C-terminal amino acid residue of the second linker polypeptide is conjugated to a cysteine residue at position 275 of the second heavy chain of the antibody.

In some embodiments, each of the first polypeptide and the second polypeptide comprise an amino acid sequence independently selected from SEQ ID NOs: 1793, 1799, 1800, 1831, and 1832. In some embodiments, each of the first linker polypeptide and the second linker polypeptide comprise an amino acid sequence independently selected from SEQ ID NOs: 1740-1746, 1841-1849, or 1852. In some embodiments, each of the first polypeptide and the second polypeptide comprise an amino acid sequence independently selected from SEQ ID NOs: 1793, 1799, 1800, 1831, and 1832, and each of the first linker polypeptide and the second linker polypeptide comprise an amino acid sequence independently selected from SEQ ID NOs: 1740-1746, 1841-1849, or 1852.

In some embodiments, the first polypeptide and the second polypeptide have identical amino acid sequences. In some embodiments, the first linker polypeptide and the second linker polypeptide have identical amino acid sequences. In some embodiments, the first polypeptide and the second polypeptide have identical amino acid sequences, and the first linker polypeptide and the second linker polypeptide have identical amino acid sequences.

Also provided herein is a molecule comprising:
a first polypeptide and a second polypeptide that agonize a glucagon receptor (“GCGR”), wherein the first polypeptide and the second polypeptide each comprise the amino acid sequence of SEQ ID NO: 1615;
a first linker polypeptide and a second linker polypeptide, wherein the first linker polypeptide and the second linker polypeptide each comprise the amino acid sequence of SEQ ID NO: 1629; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Also provided herein is a molecule comprising:
a first polypeptide and a second polypeptide that agonize a glucagon receptor (“GCGR”), wherein the first polypeptide and the second polypeptide each comprise the amino acid sequence of SEQ ID NO: 1626;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Also provided herein is a molecule comprising:
a first polypeptide and a second polypeptide that agonize a glucagon receptor (“GCGR”), wherein the first polypeptide and the second polypeptide each comprise the amino acid sequence of SEQ ID NO: 1592;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 24 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 24 of the second polypeptide is covalently linked to a C-terminus of the second linker polypeptide; and
   an N-terminus of the second linker polypeptide is conjugated to a cysteine residue at position 275 of the second heavy chain of the antibody.

Also provided herein is a molecule comprising:
a first polypeptide and a second polypeptide that agonize a glucagon receptor (“GCGR”), wherein the first polypeptide and the second polypeptide each comprise the amino acid sequence of SEQ ID NO: 1626;
a first linker polypeptide and a second linker polypeptide, wherein the first linker polypeptide and the second linker polypeptide each comprise the amino acid sequence of SEQ ID NO: 1629; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Also provided herein is a molecule comprising:
a first polypeptide and a second polypeptide that agonize a glucagon receptor (“GCGR”), wherein the first polypeptide and the second polypeptide each comprise the amino acid sequence of SEQ ID NO: 1587;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Also provided herein is a molecule comprising:
a first polypeptide and a second polypeptide that agonize a glucagon receptor (“GCGR”), wherein the first polypeptide and the second polypeptide each comprise the amino acid sequence of SEQ ID NO: 1587;
a first linker polypeptide and a second linker polypeptide, wherein the first linker polypeptide and the second linker polypeptide each comprise the amino acid sequence of SEQ ID NO: 1630; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Also provided herein is a molecule comprising:
a first polypeptide and a second polypeptide that agonize a glucagon receptor (“GCGR”), wherein the first polypeptide and the second polypeptide each comprise the amino acid sequence of SEQ ID NO: 1822;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 24 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 24 of the second polypeptide is covalently linked to a C-terminus of the second linker polypeptide; and
   an N-terminus of the second linker polypeptide is conjugated to a cysteine residue at position 275 of the second heavy chain of the antibody.

Also provided herein is a molecule comprising:
a first polypeptide and a second polypeptide that agonize a glucagon receptor (“GCGR”), wherein the first polypeptide and the second polypeptide each comprise the amino acid sequence of SEQ ID NO: 1825;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Also provided herein is a molecule comprising:
a first polypeptide and a second polypeptide that agonize a glucagon receptor (“GCGR”), wherein the first polypeptide and the second polypeptide each comprise the amino acid sequence of SEQ ID NO: 1826;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Also provided herein is a molecule comprising:
a first polypeptide and a second polypeptide that agonize a glucagon receptor (“GCGR”), wherein the first polypeptide and the second polypeptide each comprise the amino acid sequence of SEQ ID NO: 1818;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

Also provided herein is a molecule that is a product of a thiol-bromoacetyl reaction between a structure as depicted in FIG. 9 and a thiol group of a cysteine residue of an anti-GIPR antibody. In some embodiments, the molecule is a product of a thiol-bromoacetyl reaction between a structure as depicted in FIG. 9 and a thiol group of a cysteine residue at position 275 of SEQ ID NO: 1571.

Also provided herein is a molecule prepared by condensing two structures as depicted in FIG. 9 with an anti-GIPR antibody comprising two heavy chains, each comprising the amino acid sequence of SEQ ID NO: 1571, and two light chains, each comprising the amino acid sequence of SEQ ID NO: 388, wherein each structure as depicted in FIG. 9 is covalently linked to the anti-GIPR antibody via a thiol-bromoacetyl reaction between its bromoacetylated N-terminus and a thiol group of a cysteine residue at position 275 of a heavy chain (one molecule per heavy chain).

Also provided herein is a molecule comprising the structure of FIG. 10, wherein the squiggly line represents a connection point between the structure of FIG. 10 and a sulfur atom of a cysteine residue of an anti-GIPR antibody.

In some embodiments, the molecule comprises the structure of FIG. 10, wherein the squiggly line represents a connection point between the structure of FIG. 10 and a sulfur atom of a cysteine residue at position 275 of SEQ ID NO: 1571.

In some embodiments, the molecule comprises:
a first structure of FIG. 10, wherein the squiggly line represents a connection point between the first structure of FIG. 10 and a sulfur atom of a cysteine residue at position 275 of a first heavy chain, wherein the first heavy chain comprises the amino acid sequence of SEQ ID NO: 1571;
a second structure of FIG. 10, wherein the squiggly line represents a connection point between the second structure of FIG. 10 and a sulfur atom of a cysteine residue at position 275 of a second heavy chain, wherein the second heavy chain comprises the amino acid sequence of SEQ ID NO: 1571; and
a first light chain and a second light chain, wherein the first light chain and the second light chain both comprise the amino acid sequence of SEQ ID NO: 388.

Also provided herein is a molecule that is a product of a thiol-bromoacetyl reaction between a structure as depicted in FIG. 11 and a thiol group of a cysteine residue of an anti-GIPR antibody. In some embodiments, the molecule is a product of a thiol-bromoacetyl reaction between a structure as depicted in FIG. 11 and a thiol group of a cysteine residue at position 275 of SEQ ID NO: 1571.

Also provided herein is a molecule prepared by condensing two structures as depicted in FIG. 11 with an anti-GIPR antibody comprising two heavy chains, each comprising the amino acid sequence of SEQ ID NO: 1571, and two light chains, each comprising the amino acid sequence of SEQ ID NO: 388, wherein each structure as depicted in FIG. 11 is covalently linked to the anti-GIPR antibody via a thiol-bromoacetyl reaction between its bromoacetylated N-terminus and a thiol group of a cysteine residue at position 275 of a heavy chain (one molecule per heavy chain).

Also provided herein is a molecule comprising the structure of FIG. 12, wherein the squiggly line represents a connection point between the structure of FIG. 12 and a sulfur atom of a cysteine residue of an anti-GIPR antibody.

In some embodiments, the molecule comprises the structure of FIG. 12, wherein the squiggly line represents a connection point between the structure of FIG. 12 and a sulfur atom of a cysteine residue at position 275 of SEQ ID NO: 1571.

In some embodiments, the molecule comprises:
a first structure of FIG. 12, wherein the squiggly line represents a connection point between the first structure of FIG. 12 and a sulfur atom of a cysteine residue at position 275 of a first heavy chain, wherein the first heavy chain comprises the amino acid sequence of SEQ ID NO: 1571;
a second structure of FIG. 12, wherein the squiggly line represents a connection point between the second structure of FIG. 12 and a sulfur atom of a cysteine residue at position 275 of a second heavy chain, wherein the second heavy chain comprises the amino acid sequence of SEQ ID NO: 1571; and
a first light chain and a second light chain, wherein the first light chain and the second light chain both comprise the amino acid sequence of SEQ ID NO: 388.

Also provided herein is a molecule that is a product of a thiol-bromoacetyl reaction between a structure as depicted in FIG. 13 and a thiol group of a cysteine residue of an anti-GIPR antibody. In some embodiments, the molecule is a product of a thiol-bromoacetyl reaction between a structure as depicted in FIG. 13 and a thiol group of a cysteine residue at position 275 of SEQ ID NO: 1571.

Also provided herein is a molecule prepared by condensing two structures as depicted in FIG. 13 with an anti-GIPR antibody comprising two heavy chains, each comprising the amino acid sequence of SEQ ID NO: 1571, and two light chains, each comprising the amino acid sequence of SEQ ID NO: 388, wherein each structure as depicted in FIG. 13 is covalently linked to the anti-GIPR antibody via a thiol-bromoacetyl reaction between its bromoacetylated N-terminus and a thiol group of a cysteine residue at position 275 of a heavy chain (one molecule per heavy chain).

Also provided herein is a molecule comprising the structure of FIG. 14, wherein the squiggly line represents a connection point between the structure of FIG. 14 and a sulfur atom of a cysteine residue of an anti-GIPR antibody.

In some embodiments, the molecule comprises the structure of FIG. 14, wherein the squiggly line represents a connection point between the structure of FIG. 14 and a sulfur atom of a cysteine residue at position 275 of SEQ ID NO: 1571.

In some embodiments, the molecule comprises:
a first structure of FIG. 14, wherein the squiggly line represents a connection point between the first structure of FIG. 14 and a sulfur atom of a cysteine residue at position 275 of a first heavy chain, wherein the first heavy chain comprises the amino acid sequence of SEQ ID NO: 1571;
a second structure of FIG. 14, wherein the squiggly line represents a connection point between the second structure of FIG. 14 and a sulfur atom of a cysteine residue at position 275 of a second heavy chain, wherein the second heavy chain comprises the amino acid sequence of SEQ ID NO: 1571; and
a first light chain and a second light chain, wherein the first light chain and the second light chain both comprise the amino acid sequence of SEQ ID NO: 388.

Also provided herein is a molecule that is a product of a thiol-bromoacetyl reaction between a structure as depicted in FIG. 15 and a thiol group of a cysteine residue of an anti-GIPR antibody. In some embodiments, the molecule is a product of a thiol-bromoacetyl reaction between a structure as depicted in FIG. 15 and a thiol group of a cysteine residue at position 275 of SEQ ID NO: 1571.

Also provided herein is a molecule prepared by condensing two structures as depicted in FIG. 15 with an anti-GIPR antibody comprising two heavy chains, each comprising the amino acid sequence of SEQ ID NO: 1571, and two light chains, each comprising the amino acid sequence of SEQ ID NO: 388, wherein each structure as depicted in FIG. 15 is covalently linked to the anti-GIPR antibody via a thiol-bromoacetyl reaction between its bromoacetylated N-terminus and a thiol group of a cysteine residue at position 275 of a heavy chain (one molecule per heavy chain).

Also provided herein is a molecule comprising the structure of FIG. 16, wherein the squiggly line represents a connection point between the structure of FIG. 16 and a sulfur atom of a cysteine residue of an anti-GIPR antibody.

In some embodiments, the molecule comprises the structure of FIG. 16, wherein the squiggly line represents a connection point between the structure of FIG. 16 and a sulfur atom of a cysteine residue at position 275 of SEQ ID NO: 1571.

In some embodiments, the molecule comprises:
a first structure of FIG. 16, wherein the squiggly line represents a connection point between the first structure of FIG. 16 and a sulfur atom of a cysteine residue at position 275 of a first heavy chain, wherein the first heavy chain comprises the amino acid sequence of SEQ ID NO: 1571;
a second structure of FIG. 16, wherein the squiggly line represents a connection point between the second structure of FIG. 16 and a sulfur atom of a cysteine residue at position 275 of a second heavy chain, wherein the second heavy chain comprises the amino acid sequence of SEQ ID NO: 1571; and
a first light chain and a second light chain, wherein the first light chain and the second light chain both comprise the amino acid sequence of SEQ ID NO: 388.

In some embodiments of the present disclosure, the structure as depicted in FIG. 10 is conjugated to an anti-GIPR antibody via a cysteine residue of the anti-GIPR antibody. Illustratively, provided herein is a molecule comprising a structure as depicted in FIG. 10 and an antibody that specifically binds to a glucose-dependent insulinotropic polypeptide receptor (“GIPR”) (e.g., an anti-GIPR antibody as described herein). In some embodiments, the structure as depicted in FIG. 10 is conjugated to a cysteine residue of the antibody. In some embodiments, the antibody comprises a light chain and a heavy chain, wherein the light chain comprises the amino acid sequence of SEQ ID NO: 388 and the heavy chain comprises the amino acid sequence of SEQ ID NO: 1571, and, in some embodiments, the structure as depicted in FIG. 10 is conjugated to a cysteine residue at position 275 of the heavy chain. Also provided herein is a molecule comprising a first structure as depicted in FIG. 10, a second structure as depicted in FIG. 10, and an antibody that specifically binds to a glucose-dependent insulinotropic polypeptide receptor (“GIPR”) (e.g., an anti-GIPR antibody as described herein). In some embodiments, the first and second structures as depicted in FIG. 10 are each conjugated to a cysteine residue of the antibody. In some embodiments, the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571, and further wherein the first structure as depicted in FIG. 10 is conjugated to a cysteine residue at position 275 of the first heavy chain and the second structure as depicted in FIG. 10 is conjugated to a cysteine residue at position 275 of the second heavy chain.

In some embodiments of the present disclosure, the structure as depicted in FIG. 12 is conjugated to an anti-GIPR antibody via a cysteine residue of the anti-GIPR antibody. Illustratively, provided herein is a molecule comprising a structure as depicted in FIG. 12 and an antibody that specifically binds to a glucose-dependent insulinotropic polypeptide receptor (“GIPR”) (e.g., an anti-GIPR antibody as described herein). In some embodiments, the structure as depicted in FIG. 12 is conjugated to a cysteine residue of the antibody. In some embodiments, the antibody comprises a light chain and a heavy chain, wherein the light chain comprises the amino acid sequence of SEQ ID NO: 388 and the heavy chain comprises the amino acid sequence of SEQ ID NO: 1571, and, in some embodiments, the structure as depicted in FIG. 12 is conjugated to a cysteine residue at position 275 of the heavy chain. Also provided herein is a molecule comprising a first structure as depicted in FIG. 12, a second structure as depicted in FIG. 12, and an antibody that specifically binds to a glucose-dependent insulinotropic polypeptide receptor (“GIPR”) (e.g., an anti-GIPR antibody as described herein). In some embodiments, the first and second structures as depicted in FIG. 12 are each conjugated to a cysteine residue of the antibody. In some embodiments, the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571, and further wherein the first structure as depicted in FIG. 12 is conjugated to a cysteine residue at position 275 of the first heavy chain and the second structure as depicted in FIG. 12 is conjugated to a cysteine residue at position 275 of the second heavy chain.

In some embodiments of the present disclosure, the structure as depicted in FIG. 14 is conjugated to an anti-GIPR antibody via a cysteine residue of the anti-GIPR antibody. Illustratively, provided herein is a molecule comprising a structure as depicted in FIG. 14 and an antibody that specifically binds to a glucose-dependent insulinotropic polypeptide receptor (“GIPR”) (e.g., an anti-GIPR antibody as described herein). In some embodiments, the structure as depicted in FIG. 14 is conjugated to a cysteine residue of the antibody. In some embodiments, the antibody comprises a light chain and a heavy chain, wherein the light chain comprises the amino acid sequence of SEQ ID NO: 388 and the heavy chain comprises the amino acid sequence of SEQ ID NO: 1571, and, in some embodiments, the structure as depicted in FIG. 14 is conjugated to a cysteine residue at position 275 of the heavy chain. Also provided herein is a molecule comprising a first structure as depicted in FIG. 14, a second structure as depicted in FIG. 14, and an antibody that specifically binds to a glucose-dependent insulinotropic polypeptide receptor (“GIPR”) (e.g., an anti-GIPR antibody as described herein). In some embodiments, the first and second structures as depicted in FIG. 14 are each conjugated to a cysteine residue of the antibody. In some embodiments, the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571, and further wherein the first structure as depicted in FIG. 14 is conjugated to a cysteine residue at position 275 of the first heavy chain and the second structure as depicted in FIG. 14 is conjugated to a cysteine residue at position 275 of the second heavy chain.

In some embodiments of the present disclosure, the structure as depicted in FIG. 16 is conjugated to an anti-GIPR antibody via a cysteine residue of the anti-GIPR antibody. Illustratively, provided herein is a molecule comprising a structure as depicted in FIG. 16 and an antibody that specifically binds to a glucose-dependent insulinotropic polypeptide receptor (“GIPR”) (e.g., an anti-GIPR antibody as described herein). In some embodiments, the structure as depicted in FIG. 16 is conjugated to a cysteine residue of the antibody. In some embodiments, the antibody comprises a light chain and a heavy chain, wherein the light chain comprises the amino acid sequence of SEQ ID NO: 388 and the heavy chain comprises the amino acid sequence of SEQ ID NO: 1571, and, in some embodiments, the structure as depicted in FIG. 16 is conjugated to a cysteine residue at position 275 of the heavy chain. Also provided herein is a molecule comprising a first structure as depicted in FIG. 16, a second structure as depicted in FIG. 16, and an antibody that specifically binds to a glucose-dependent insulinotropic polypeptide receptor (“GIPR”) (e.g., an anti-GIPR antibody as described herein). In some embodiments, the first and second structures as depicted in FIG. 16 are each conjugated to a cysteine residue of the antibody. In some embodiments, the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571, and further wherein the first structure as depicted in FIG. 16 is conjugated to a cysteine residue at position 275 of the first heavy chain and the second structure as depicted in FIG. 16 is conjugated to a cysteine residue at position 275 of the second heavy chain.

In some embodiments, the anti-GIPR×GCGR agonist conjugates described herein have a hGCGR EC50 of less than or equal to 1 nM.

In some embodiments, the anti-GIPR×GCGR agonist conjugates described herein have a hGCGR EC₅₀ of less than or equal to 500 pM (e.g., less than or equal to 475 pM, less than or equal to 450 pM, less than or equal to 425 pM, less than or equal to 400 pM, less than or equal to 375 pM, less than or equal to 350 pM, less than or equal to 325 pM, less than or equal to 300 pM, less than or equal to 275 pM, less than or equal to 250 pM, less than or equal to 225 pM, less than or equal to 200 pM, less than or equal to 175 pM, less than or equal to 150 pM, less than or equal to 125 pM, less than or equal to 100 pM). In some embodiments, the hGCGR EC₅₀ is measured in a functional assay described in “EXAMPLE 1: GCG Receptor Agonist Activity.”

In some embodiments, the anti-GIPR×GCGR agonist conjugates described herein have a hGCGR EC₅₀ of 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 155 pM, 200 pM, 205 pM, 210 pM, 215 pM, 220 pM, 225 pM, 230 pM, 235 pM, 240 pM, 245 pM, 250 pM, 255 pM, 260 pM, 265 pM, 270 pM, 275 pM, 280 pM, 285 pM, 290 pM, 295 pM, 300 pM, 305 pM, 310 pM, 315 pM, 320 pM, 325 pM, 330 pM, 335 pM, 340 pM, 345 pM, 350 pM, 355 pM, 360 pM, 365 pM, 370 pM, 375 pM, 380 pM, 385 pM, 390 pM, 395 pM, 400 pM, 405 pM, 410 pM, 415 pM, 420 pM, 425 pM, 430 pM, 435 pM, 440 pM, 445 pM, 450 pM, 455 pM, 460 pM, 465 pM, 470 pM, 475 pM, 480 pM, 485 pM, 490 pM, 495 pM, or 500 pM. In some embodiments, the hGCGR EC₅₀ is measured in a functional assay described in “EXAMPLE 1: GCG Receptor Agonist Activity.”

In some embodiments, the conjugate has a human glucagon-like peptide-1 receptor (“hGLP-1R”) EC₅₀:hGCGR EC₅₀ ratio of at least 30:1. In some embodiments, the conjugate has a hGLP-1R EC₅₀:hGCGR EC₅₀ ratio of at least 40:1. In some embodiments, the conjugate has a hGLP-1R EC₅₀:hGCGR EC₅₀ ratio of at least 50:1. In some embodiments, the conjugate has a hGLP-1R EC₅₀:hGCGR EC₅₀ ratio of at least 60:1. In some embodiments, the conjugate has a hGLP-1R EC₅₀:hGCGR EC₅₀ ratio of at least 70:1. In some embodiments, the conjugate has a hGLP-1R EC₅₀:hGCGR EC₅₀ ratio of at least 80:1. In some embodiments, the conjugate has a hGLP-1R EC₅₀:hGCGR EC₅₀ ratio of at least 90:1. In some embodiments, the conjugate has a hGLP-1R EC₅₀:hGCGR EC₅₀ ratio of at least 100:1. In some embodiments, the conjugate has a hGLP-1R EC₅₀:hGCGR EC₅₀ ratio of at least 150:1. In some embodiments, the conjugate has a hGLP-1R EC₅₀:hGCGR EC₅₀ ratio of at least 200:1. In some embodiments, the conjugate has a hGLP-1R EC₅₀:hGCGR EC₅₀ ratio of at least 250:1. In some embodiments, the conjugate has a hGLP-1R EC₅₀:hGCGR EC₅₀ ratio of at least 300:1.

In some embodiments, the conjugate has a hGLP-1R EC₅₀:hGCGR EC₅₀ ratio of at least 350:1. In some embodiments, the conjugate has a hGLP-1R EC₅₀:hGCGR EC₅₀ ratio of at least 400:1. In some embodiments, the conjugate has a hGLP-1R EC₅₀:hGCGR EC₅₀ ratio of at least 450:1. In some embodiments, the conjugate has a hGLP-1R EC₅₀:hGCGR EC₅₀ ratio of at least 500:1. In some embodiments, the conjugate has a hGLP-1R EC₅₀:hGCGR EC₅₀ ratio of at least 550:1. In some embodiments, the conjugate has a hGLP-1R EC₅₀:hGCGR EC₅₀ ratio of at least 650:1. In some embodiments, the conjugate has a hGLP-1R EC₅₀:hGCGR EC₅₀ ratio of at least 700:1. In some embodiments, the conjugate has a hGLP-1R EC₅₀:hGCGR EC₅₀ ratio of at least 750:1. In some embodiments, the conjugate has a hGLP-1R EC₅₀:hGCGR EC₅₀ ratio of at least 800:1. In some embodiments, the conjugate has a hGLP-1R EC₅₀:hGCGR EC₅₀ ratio of at least 850:1. In some embodiments, the conjugate has a hGLP-1R EC₅₀:hGCGR EC₅₀ ratio of at least 900:1. In some embodiments, the conjugate has a hGLP-1R EC₅₀:hGCGR EC₅₀ ratio of at least 950:1. In some embodiments, the conjugate has a hGLP-1R EC₅₀:hGCGR EC₅₀ ratio of at least 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

In some embodiments, the conjugate has a hGLP-1R EC₅₀:hGCGR EC₅₀ ratio of 30:1, 40:1, 45:1, 50:1, 55:1, 60:1, 65:1, 70:1, 75:1, 80:1, 85:1, 90:1, 95:1, 100:1, 125:1, 150:1, 175:1, 200:1, 225:1, 250:1, 275:1, 300:1, 325:1, 350:1, 375:1, 400:1, 425:1, 450:1, 475:1, 500:1, 525:1, 550:1, 575:1, 600:1, 625:1, 650:1, 675:1, 700:1, 725:1, 750:1, 775:1, 800:1, 825:1, 850:1, 875:1, 900:1, 925:1, 950:1, 975:1, or 1000:1. In some embodiments, the hGCGR EC₅₀ is measured in a functional assay described in “EXAMPLE 1: GCG Receptor Agonist Activity” and the hGLP-1R EC₅₀ is measured in a functional assay described in “EXAMPLE 2: GLP-1 Receptor Agonist Activity.”

In some embodiments, the anti-GIPR×GCGR agonist conjugates described herein have a hGIPR IC₅₀ of less than or equal to 500 nM (e.g., less than or equal to 475 nM, less than or equal to 450 nM, less than or equal to 425 nM, less than or equal to 400 nM, less than or equal to 375 nM, less than or equal to 350 nM, less than or equal to 325 nM, less than or equal to 300 nM, less than or equal to 275 nM, less than or equal to 250 nM, less than or equal to 225 nM, less than or equal to 200 nM, less than or equal to 175 nM, less than or equal to 150 nM, less than or equal to 125 nM, less than or equal to 100 nM). In some embodiments, the hGIPR IC₅₀ is measured in a functional assay described in “EXAMPLE 3: GIPR Antagonist Activity.”

In some embodiments, the anti-GIPR×GCGR agonist conjugates described herein have a hGIPR IC₅₀ of 5 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 105 nM, 110 nM, 115 nM, 120 nM, 125 nM, 130 nM, 135 nM, 140 nM, 145 nM, 150 nM, 155 nM, 200 nM, 205 nM, 210 nM, 215 nM, 220 nM, 225 nM, 230 nM, 235 nM, 240 nM, 245 nM, 250 nM, 255 nM, 260 nM, 265 nM, 270 nM, 275 nM, 280 nM, 285 nM, 290 nM, 295 nM, 300 nM, 305 nM, 310 nM, 315 nM, 320 nM, 325 nM, 330 nM, 335 nM, 340 nM, 345 nM, 350 nM, 355 nM, 360 nM, 365 nM, 370 nM, 375 nM, 380 nM, 385 nM, 390 nM, 395 nM, 400 nM, 405 nM, 410 nM, 415 nM, 420 nM, 425 nM, 430 nM, 435 nM, 440 nM, 445 nM, 450 nM, 455 nM, 460 nM, 465 nM, 470 nM, 475 nM, 480 nM, 485 nM, 490 nM, 495 nM, or 500 nM. In some embodiments, the hGIPR IC₅₀ is measured in a functional assay described in “EXAMPLE 3: GIPR Antagonist Activity.”

### Linker Moieties and Conjugation Methods

Linker moieties may be used in certain conjugates disclosed herein to link a glucagon or glucagon analog to another molecule, such as a half-life extending domain (e.g., an Fc-containing polypeptide) or an antigen-binding protein (e.g., an anti-GIPR antibody). When present, the chemical structure of the linker moiety is not critical, since it serves primarily as a spacer to position, join, connect, or optimize presentation or position of one functional moiety in relation to one or more other functional moieties. In some embodiments, a linker moiety, if present, can be made up of amino acids linked together by peptide bonds and referred to as a peptidyl linker or a linker polypeptide. In some embodiments, a linker moiety, if present, can be independently the same or different from any other linker moiety, or linker moieties, that may be present in a disclosed molecule. In some embodiments, the linker moiety can be chemically derivatized (such as, e.g., at the N-terminus or the C-terminus), e.g., when a functional group was installed to facilitate covalent joining or conjugation of the linker moiety to another molecule.

As stated above, in some embodiments, the linker moiety, if present, can be “peptidyl” in nature (i.e., made up of amino acids linked together by peptide bonds) and made up in length, preferably, of from 1 up to about 40 amino acid residues, more preferably, of from 1 up to about 20 amino acid residues, and most preferably of from 1 to about 10 amino acid residues. Preferably, but not necessarily, the amino acid residues in the linker are from among the twenty canonical amino acids, more preferably, cysteine, glycine, alanine, proline, asparagine, glutamine, and/or serine. Even more preferably, a peptidyl linker can be made up of a majority of amino acids that are sterically unhindered, such as glycine, serine, and alanine linked by a peptide bond. In some embodiments, if present, a peptidyl linker is selected that avoids rapid proteolytic turnover in circulation in vivo.

Non-limiting examples of linker moieties that can be used in molecules of the present disclosure include linker polypeptides comprising the amino acid sequences presented in Table 18A or Table 18B. In Table 18A, Pra is shorthand for L-propargylglycine and Hyp is shorthand for 4-hydroxyproline. In some embodiments, the linker polypeptide comprises an amino acid sequence selected from the amino acid sequences of Table 18A (e.g., any of SEQ ID NOs: 1628-1630; SEQ ID NO: 1628, SEQ TD NO: 1629, SEQ TD NO: 1630), wherein the linker polypeptide is derivatized to facilitate conjugation. In some embodiments, the linker polypeptide comprises an amino acid sequence selected from the amino acid sequences of Table 18A (e.g., any of SEQ ID NOs: 1628-1630; SEQ ID NO: 1628, SEQ TD NO: 1629, SEQ ID NO: 1630), wherein the N-terminus is derivatized to facilitate conjugation. In some embodiments, the linker polypeptide comprises an amino acid sequence selected from the amino acid sequences of Table 18A (e.g., any of SEQ TD NOs: 1628-1630; SEQ ID NO: 1628, SEQ TD NO: 1629, SEQ ID NO: 1630), wherein the N-terminus is acetylated.

In some embodiments, the linker polypeptide comprises an amino acid sequence selected from the amino acid sequences of Table 18B3, wherein the linker polypeptide is derivatized to facilitate conjugation. In some embodiments, the linker polypeptide comprises an amino acid sequence selected from the amino acid sequences of Table 18B3, wherein the N-terminus is derivatized to facilitate conjugation. In some embodiments, the linker polypeptide comprises an amino acid sequence selected from the amino acid sequences of Table 181B, wherein the N-terminus is acetylated.

TABLE 18A
Example Linker Sequences

| | SEQ |
|---|---|
| Linker Sequence | ID NO: |
| GGGGS GGGGS PAPAP APAPA PASGG | 1628 |
| GSPAP APAPA PAPAP APAPA PASGG | 1629 |
| GGGGS GGGGS GGGGS GGGGS GGGGS | 1630 |
| GSPAP APAPA PAPAP APAPA PASG | 1631 |
| GSPAP APAPA PAPAG GGGSG GGGS | 1632 |
| GSPAP APAPA PASGG GG | 1633 |
| GSPAP APAPA PASGG | 1634 |
| GSPAP APAPA SGG | 1635 |
| GGGGS GGGGS GGGGS GGGGS | 1636 |
| GSGGG GSGGG GSGGG GSGGG G | 1637 |
| GGGGS GGGGS GGGGS | 1638 |
| GSGGG GSGGG GSGGG G | 1639 |
| GAKAA AEKAA AEKAA AEKAA AEA | 1640 |
| GAKAQ ADAKA QADAK AQADA KAQAD SGG | 1641 |
| GS[Hyp][Hyp][Hyp][Hyp][Hyp][Hyp][Hyp] | 1642 |
| [Hyp][Hyp][Hyp][Hyp][Hyp]S GG | |
| G[NPeg24] | 1643 |
| G[NPeg27] | 1644 |
| GG[NPeg24] | 1645 |
| GG[NPeg6][NPeg11] | 1646 |
| GINPeg11]SPA PAPAP APAPA SGG | 1647 |
| GGGSG GGS | 1648 |
| GGGSG GGSGG GS | 1649 |
| GGGSG GGSGG GSGGG S | 1650 |
| GGGSG GGSGG GSGGG SGGGS | 1651 |
| GGGSG GGSGG GSGGG SGGGS GGGS | 1652 |
| GGGGS GGGGS | 1653 |
| GGGGS GGGGS GGGGS GGGGS GGGGS GGGGS | 1654 |
| GGG | 1655 |
| GGGG | 1656 |
| GGGGG | 1657 |
| GGGGG GG | 1658 |
| GGGGG K | 1659 |
| GGGGG KR | 1660 |
| GGGKG GGG | 1661 |
| GGGNG SGG | 1662 |
| GGGCG GGG | 1663 |
| GPNGG | 1664 |
| GGGGS | 1665 |
| GGEGG G | 1666 |
| GGEEE GGG | 1667 |
| GEEEG | 1668 |
| GEEE | 1669 |
| GGDGG G | 1670 |
| GGDDD GG | 1671 |
| GDDDG | 1672 |
| GDDD | 1673 |
| GGGGS DDSDE GSDGE DGGGG S | 1674 |
| WEWEW | 1675 |
| FEFEF | 1676 |
| EEEWW W | 1677 |
| EEEFF F | 1678 |
| WWEEE WW | 1679 |
| FFEEE FF | 1680 |
| GSGSA TGGSG STASS GSGSA TH | 1681 |
| HGSGS ATGGS GSTAS SGSGS AT | 1682 |
| AEAAA KEAAA KEAAA KAGG | 1683 |
| GSPAP APAPA PAPAP APAPA PASGG [Pra] | 1739 |
| [NPeg27]K | 1740 |
| A PAPAP APAPA PAGGG GSGGG GSK | 1741 |
| A PAPAP APAPA PAPAP APAPA K | 1742 |
| G GGGSG GGGSG GGGSG GGGSK | 1743 |
| G GGGSG GGGSG GGGSK | 1744 |
| G GSAPA PAPAP APAPA PAPAP APSGK | 1745 |
| GG GGSGG GGSGG GGSK | 1746 |

TABLE 18B
Additional Example Linker Sequences

| Linker Sequence | SEQ ID NO: |
|---|---|
| GGGGSGGGGSAPAPAPAPAPAPAK | 1841 |
| GGGGSGGGGSAPAPAPAPAPGGSK | 1842 |
| GPSGGGGSGGGGSAPAPAPAPAPAPGGSK | 1843 |
| GPSGGSAPAPAPAPAPAPGGGGSGGGGSK | 1844 |
| GPSSGAPPPSAPAPAPAPAPAPAPAPAPAPAK | 1845 |
| GPSSGAPPPSGGGGSGGGGSGGGGSK | 1846 |
| GSAPAPAPAPAPAPAPAPAPAPGGSK | 1847 |
| PSSGAPPPSAPAPAPAPAPAPAPAPAPAPAK | 1848 |
| PSSGAPPPSGGGGSGGGGSGGGGSK | 1849 |
| GQPAP APAPA PAPAP APAPA PAQGG | 1850 |
| GGGGQ GGGGQ PAPAP APAPA PAQGG | 1851 |
| G PSGSA PAPAP APAPA PAPAP APAPG GSK | 1852 |

In some embodiments, one or more amino acids of a linker polypeptide may be glycosylated.

In some embodiments, a linker polypeptide comprises a sialylation site. In some embodiments, the linker polypeptide comprises X₁X₂NX₄X₅G (SEQ ID NO: 1684), wherein X₁, X₂, X₄ and X₅ are each independently any amino acid residue.

In some embodiments, a linker polypeptide comprises a phosphorylation site. In some embodiments, the linker polypeptide comprises X₁X₂YX₄X₅G (SEQ ID NO: 1685), wherein X₁, X₂, X₄, and X₅ are each independently any amino acid residue. In some embodiments, the linker polypeptide comprises X₁X₂SX₄X₅G (SEQ ID NO: 1686), wherein X₁, X₂, X₄, and X₅ are each independently any amino acid residue. In some embodiments, the linker polypeptide comprises X₁X₂TX₄X₅G (SEQ ID NO: 1687), wherein X₁, X₂, X₄, and X₅ are each independently any amino acid residue.

Specific polypeptide linkers are provided for the purposes of illustration only. Peptidyl linkers within the scope of this disclosure may be much longer and may include other residues than those specifically recited herein. For example, a linker polypeptide can comprise, e.g., a cysteine, another thiol, or nucleophile for conjugation with a half-life extending moiety, such as, e.g., a Fc-containing polypeptide. In other embodiments, a linker polypeptide can comprise a cysteine or homocysteine residue, or other 2-amino-ethanethiol or 3-amino-propanethiol moiety for conjugation to a maleimide, iodoacetaamide, or thioester functional group. Moreover, in alternative embodiments of example embodiments of the disclosure that recite a linker polypeptide comprising the amino acid sequence of any one of SEQ ID NOs: 1628-1683, a linker polypeptide described in this section (e.g., a linker polypeptide comprising the amino acid sequence of any one of SEQ ID NOs: 1684-1687 or SEQ ID NOs: 1739-1746) can be used instead.

Alternatively, a non-peptidyl linker moiety can be used in certain molecules described herein. For example, in some embodiments, alkyl linkers such as —NH—(CH₂)ₛ—C(O)—, wherein s=2-20, can be used. These alkyl linkers may further be substituted by any non-sterically hindering group. One non-limiting example class of non-peptidyl linkers is polyethylene glycol (PEG) linkers, such as, e.g.,

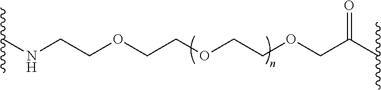

wherein n is such that the linker has a molecular weight of about 100 Daltons (Da) to about 5000 Da, preferably about 100 Da to about 500 Da.

In some embodiments, the non-peptidyl linker comprises a rigid polyheterocyclic core of controlled length. Such linkers may be chemically differentiated on either end to accommodate orthogonal coupling chemistries (i.e., azide “click”, amide coupling, thioether formation by alkylation with maleimide or haloacetamide, oxime formation, reductive amination, etc.).

Non-peptidyl linkers can be synthesized by conventional organic chemistry reactions.

The above is merely illustrative and not an exhaustive treatment of the kinds of non-peptidyl linkers that can optionally be employed in accordance with the present disclosure.

In some embodiments, a chemistry for chemoselective conjugation can be used to conjugate a polypeptide to a linker moiety. Such chemistries include, but are not limited to, copper(I)-catalyzed azide-alkyne [3+2] dipolar cycloadditions, Staudinger ligation, other acyl transfers processes, oximations, hydrazone bonding formation, and other suitable organic chemistry reactions such as cross-couplings using water-soluble palladium catalysts. (E.g., Bong et al., Chemoselective Pd(0)-catalyzed peptide coupling in water, Organic Letters 3(16):2509-11 (2001); Dibowski et al., Bioconjugation of peptides by palladium-catalyzed C—C cross-coupling in water, Angew. Chem. Int. Ed. 37(4):476-78 (1998); DeVasher et al., Aqueous-phase, palladium-catalyzed cross-coupling of aryl bromides under mild conditions, using water-soluble, sterically demanding alkylphosphines, J. Org. Chem. 69:7919-27 (2004); Shaugnessy et al., J. Org. Chem, 2003, 68, 6767-6774; Prescher, JA and Bertozzi C R, Chemistry in living system, Nature Chemical Biology 1(1); 13-21 (2005)).

Glucagon receptor agonist amino acid residues that can provide a primary amine moiety for conjugation include residues of lysine, homolysine, ornithine, α, β-diaminopropionic acid (Dap), α, β-diaminopropionoic acid (Dpr), and α, γ-diaminobutyric acid (Dab), aminobutyric acid (Abu), and α-amino-isobutyric acid (Aib). The polypeptide N-terminus also provides a useful α-amino group for conjugation as does an amidated polypeptide C-terminus.

In some embodiments, an anti-GIPR antigen-binding protein can be conjugated to a linker moiety through the side chain of an amino acid residue at an internal conjugation site, such as, e.g., a cysteinyl residue. In some embodiments, the amino acid residue, e.g., a cysteinyl residue, at the internal conjugation site that is selected can be one that occupies the same amino acid residue position in a native Fc domain sequence, or the amino acid residue can be engineered into the Fc domain sequence by substitution or insertion.

In some embodiments, an antibody conjugate of the present disclosure can be prepared using a conjugation method described in WO 2019/028382, which is incorporated by reference herein.

### Pharmaceutical Compositions

While it may be possible to administer a polypeptide or molecule disclosed herein alone in the uses described, the polypeptide or molecule will normally be administered as an active ingredient in a pharmaceutical composition. Thus, further provided herein is a pharmaceutical composition comprising a polypeptide or a molecule disclosed herein and a pharmaceutically acceptable excipient. Non-limiting examples of pharmaceutically acceptable excipients include calcium carbonate, calcium phosphate, sugars (e.g., lactose, glucose, or sucrose), starches, cellulose derivatives, gelatin, vegetable oils, polyethylene glycols, and physiologically compatible solvents. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018.

In some cases, the pharmaceutical composition described herein comprises a therapeutically effective amount of a polypeptide or molecule disclosed herein. In some embodiments, the pharmaceutical composition is made in the form of a dosage unit containing a particular amount of the active ingredient.

The polypeptides and molecules disclosed herein may be administered by any suitable route in the form of a pharmaceutical composition adapted to such a route and in a dose effective for the treatment intended. The polypeptides, molecules, and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients. For example, in some embodiments, a pharmaceutical composition disclosed herein may be provided for peripheral administration, such as parenteral (e.g., subcutaneous, intravenous, intramuscular), continuous infusion (e.g., intravenous drip, intravenous bolus, intravenous infusion), topical, nasal, or oral administration.

Pharmaceutical compositions disclosed herein may be sterilized by conventional sterilization techniques or may be sterile-filtered. In some embodiments, such compositions comprise sterile water. In some embodiments, such compositions can contain pharmaceutically acceptable auxiliary substances as required to approximate physiological conditions, such as, e.g., pH buffering agents (e.g., an acetic acid buffer).

In some embodiments, the pharmaceutical composition comprises sterile water and sodium chloride. In some embodiments, the pharmaceutical composition comprises sterile water and dextrose. In some embodiments, the pharmaceutical composition comprises sterile water, sodium chloride, and dextrose.

In some embodiments, the pharmaceutical composition comprises an aqueous carrier. In some embodiments, the aqueous carrier is an isotonic buffer solution at a pH in the range of 3.0 to 8.0 (such as, e.g., in the range of 3.0 to 5.0).

In some embodiments, the pharmaceutical composition is a parenteral composition. In some embodiments, the pharmaceutical composition is a parenteral composition for injection. In some embodiments, the pharmaceutical composition is a parenteral composition for infusion.

In some embodiments, a form of repository or “depot” slow release preparation may be used so that therapeutically effective amounts of the preparation are delivered into the bloodstream over many hours or days following subcutaneous injection, transdermal injection, or other delivery method. The desired isotonicity may be accomplished using sodium chloride or other pharmaceutically acceptable excipients, such as, e.g., dextrose, boric acid, sodium tartrate, propylene glycol, polyols (such as, e.g., mannitol and sorbitol), or other inorganic or organic solutes.

### Treatment Methods

The present disclosure provides polypeptides that agonize a glucagon receptor (“GCGR”) and molecules comprising such polypeptides and a half-life extending domain (e.g., an Fc-containing polypeptide), including molecules comprising a polypeptide that agonizes a GCGR and an antibody that specifically binds to a glucose-dependent insulinotropic polypeptide receptor (“GIPR”). Without intending to be bound by any particular theory, the polypeptides and molecules of the disclosure can, in some cases, increase energy expenditure and improve circulating insulin and glucose concentrations, leading to reduced body weight, including reduced fat mass. Besides being useful for human treatment, the polypeptides and molecules provided herein may be useful for veterinary treatment of companion animals, exotic animals, and farm animals, including mammals, rodents, and the like. For example, animals including horses, dogs, and cats may be treated with polypeptides, molecules, and pharmaceutical compositions provided herein.

The polypeptides, molecules, and pharmaceutical compositions disclosed herein may be administered to a subject in a dose effective for the treatment intended and through any suitable routable, such as, e.g., by intravenous (IV) injection, intraperitoneal (IP) injection, subcutaneous injection, intramuscular injection, or orally in the form of a tablet or liquid formation.

In some embodiments, as disclosed elsewhere herein, a method of treating a patient is provided. In some embodiments, the method comprises administering a therapeutic amount of a polypeptide or molecule disclosed herein to a patient. In some embodiments, the patient is overweight or obese. In some embodiments, the subject has a body mass index (BMI) of at least 25.0 kg/m² (e.g., in the range of 25.0 kg/m² to 29.9 kg/m², inclusive of the endpoints; in the range of 30.0 kg/m² to 40.0 kg/m², inclusive of the endpoints; at least 30.0 kg/m²).

Another aspect of the disclosure provides a polypeptide or molecule disclosed herein for use in therapy. In some cases, the use is in weight management. In some cases, the use is in treating obesity. In some cases, the polypeptide or molecule may be used in acute therapy. In other cases, the polypeptide or molecule may be used in chronic therapy. Yet another aspect of the disclosure provides a use of a polypeptide or molecule disclosed herein in the manufacture of a medicament. In some cases, the medicament is for use in weight management. In some cases, the medicament is for use in treating obesity.

Still another aspect of the disclosure provides a method of treating obesity in a subject in need thereof, the method comprising administering a polypeptide, molecule, or pharmaceutical composition disclosed herein to the subject.

In some embodiments, the administration lowers blood glucose, insulin, triglyceride, or cholesterol levels; reduces body weight; or improves glucose tolerance, energy expenditure, or insulin sensitivity. In some embodiments, the administration lowers blood glucose, insulin, triglyceride, or cholesterol levels. In some embodiments, the administration lowers blood glucose levels. In some embodiments, the administration lowers insulin levels. In some embodiments, the administration lowers triglyceride levels. In some embodiments, the administration lowers cholesterol levels. In some embodiments, the administration reduces body weight. In some embodiments, the administration improves glucose tolerance, energy expenditure, or insulin sensitivity. In some embodiments, the administration improves glucose tolerance. In some embodiments, the administration improves energy expenditure. In some embodiments, the administration improves insulin sensitivity.

In some embodiments, prior to the administering, the subject has received prior treatment with a GLP-1 agonist (e.g., semaglutide). In other embodiments, prior to the administering, the subject has not received prior treatment with a GLP-1 agonist (e.g., semaglutide).

In some embodiments, the subject also receives a GLP-1 agonist treatment (e.g., semaglutide) during the administration. In other embodiments, the subject does not receive a GLP-1 agonist treatment (e.g., semaglutide) during the administration.

In some embodiments, prior to the administering, the subject has received prior treatment with a GLP-1 agonist (e.g., semaglutide), and the subject also receives a GLP-1 agonist treatment (e.g., semaglutide) during the administration.

In some embodiments, prior to the administering, the subject has not received prior treatment with a GLP-1 agonist (e.g., semaglutide), but the subject also receives a GLP-1 agonist treatment (e.g., semaglutide) during the administration.

In some embodiments, prior to the administering, the subject has received prior treatment with a GLP-1 agonist (e.g., semaglutide), but the subject does not receive a GLP-1 agonist treatment (e.g., semaglutide) during the administration.

In some embodiments, prior to the administering, the subject has not received prior treatment with a GLP-1 agonist (e.g., semaglutide), and the subject does not receive a GLP-1 agonist treatment (e.g., semaglutide) during the administration.

In some embodiments, prior to the administering, the subject is experiencing a weight-loss plateau. In some embodiments, prior to the administering, the subject is experiencing a weight-loss plateau while receiving treatment with a GLP-1 agonist (e.g., semaglutide).

In some embodiments, prior to the administering, the subject is experiencing a weight-loss plateau while receiving treatment with a GLP-1 agonist (e.g., semaglutide), and the subject also receives a GLP-1 agonist treatment (e.g., semaglutide) during the administration.

In some embodiments, prior to the administering, the subject is experiencing a weight-loss plateau while receiving treatment with a GLP-1 agonist (e.g., semaglutide), and the subject does not receive a GLP-1 agonist treatment (e.g., semaglutide) during the administration.

In some embodiments, prior to the administering, the subject has experienced a change in body weight of less than or equal to 2.5% (e.g., ≤2.4%, ≤2.3%, ≤2.2%, ≤2.1%, ≤2.0%, ≤1.9%, ≤1.8%, ≤1.7%, ≤1.6%, ≤1.5%, ≤1.4%, ≤1.3%, ≤1.2%, ≤1.1%, ≤1.0%, ≤0.9%, ≤0.8%, ≤0.7%, ≤0.6%, ≤0.5%, 0.4% 0.3% 0.2%, ≤0.1%; 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration. In some embodiments, the baseline is the subject's body weight 30-days prior to the administration. In some embodiments, the baseline is the subject's body weight prior to a first dose of a GLP-1 agonist (semaglutide).

In some embodiments, prior to the administering, the subject has experienced a change in body weight of less than or equal to 2.5% (e.g., ≤2.4%, ≤2.3%, ≤2.2%, ≤2.1%, ≤2.0%, ≤1.9%, ≤1.8%, ≤1.7%, ≤1.6%, ≤1.5%, ≤1.4%, ≤1.3%, ≤1.2%, ≤1.1%, ≤1.0%, ≤0.9%, ≤0.8%, ≤0.7%, ≤0.6%, ≤0.5% 0.4%, ≤0.3%, ≤0.2%, ≤0.1%; 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration while receiving treatment with a GLP-1 agonist (e.g., semaglutide). In some embodiments, the baseline is the subject's body weight 30-days prior to the administration. In some embodiments, the baseline is the subject's body weight prior to a first dose of a GLP-1 agonist.

In some embodiments, prior to the administering, the subject has experienced a change in body weight of less than or equal to 2.5% (e.g., ≤2.4%, ≤2.3%, ≤2.2%, ≤2.1%, ≤2.0%, ≤1.9%, ≤1.8%, ≤1.7%, ≤1.6%, ≤1.5%, ≤1.4%, ≤1.3%, ≤1.2%, ≤1.1%, ≤1.0%, ≤0.9%, ≤0.8%, ≤0.7%, ≤0.6%, ≤0.5%, ≤0.4%, ≤0.3%, ≤0.2%, ≤0.1%; 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration while receiving treatment with a GLP-1 agonist (e.g., semaglutide), and the subject also receives a GLP-1 agonist treatment (e.g., semaglutide) during the administration.

In some embodiments, prior to the administering, the subject has experienced a change in body weight of less than or equal to 2.5% (e.g., ≤2.4%, ≤2.3%, ≤2.2%, ≤2.1%, ≤2.0%, ≤1.9%, ≤1.8%, ≤1.7%, ≤1.6%, ≤1.5%, ≤1.4%, ≤1.3%, ≤1.2%, ≤1.1%, ≤1.0%, ≤0.9%, ≤0.8%, ≤0.7%, ≤0.6%, ≤0.5%, ≤0.4%, ≤0.3%, ≤0.2%, ≤0.1%; 2.4%, ≤2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration while receiving treatment with a GLP-1 agonist (e.g., semaglutide), and the subject does not receive a GLP-1 agonist treatment (e.g., semaglutide) during the administration.

In some embodiments, the subject has a body mass index (BMI) of at least 30.0 kg/m² (e.g., 30 kg/m², 30.5 kg/m², 31 kg/m², 31.5 kg/m², 32 kg/m², 32.5 kg/m², 33 kg/m², 33.5 kg/m², 34 kg/m², 34.5 kg/m², 35 kg/m², 35.5 kg/m², 36 kg/m², 36.5 kg/m², 37 kg/m², 37.5 kg/m², 38 kg/m², 38.5 kg/m², 39 kg/m², 39.5 kg/m², 40 kg/m²).

In some embodiments, the subject has a body mass index (BMI) in the range of 30.0 kg/m² to 40.0 kg/m², inclusive of the endpoints.

In some embodiments, the subject is a human subject.

In some embodiments, the administration reduces body weight. In some embodiments, the administration reduces body weight gain. In some embodiments, the administration reduces fat mass.

In some embodiments, the administration provides one or more sustained beneficial effect(s).

Another aspect of the disclosure provides a polypeptide, molecule, or pharmaceutical composition disclosed herein for use in treating obesity.

Still another aspect of the disclosure provides use of a polypeptide, molecule, or pharmaceutical composition disclosed herein in the manufacture of a medicament for treating obesity.

Another aspect of the disclosure provides a method of stimulating insulin secretion from a cell, the method comprising contacting the cell with a polypeptide, molecule, or pharmaceutical composition disclosed herein. In some embodiments, the cell is a human pancreatic microislet cell.

Yet another aspect of the disclosure provides a method of reducing body weight and/or food intake in a subject in need thereof, the method comprising administering a polypeptide, molecule, or pharmaceutical composition disclosed herein to the subject. In some embodiments, the administration reduces body weight. In some embodiments, the administration reduces food intake. In some embodiments, the administration reduces body weight and food intake.

In some embodiments, prior to the administering, the subject has received prior treatment with a GLP-1 agonist (e.g., semaglutide). In other embodiments, prior to the administering, the subject has not received prior treatment with a GLP-1 agonist (e.g., semaglutide).

In some embodiments, the subject also receives a GLP-1 agonist treatment (e.g., semaglutide) during the administration. In other embodiments, the subject does not receive a GLP-1 agonist treatment (e.g., semaglutide) during the administration.

In some embodiments, prior to the administering, the subject has received prior treatment with a GLP-1 agonist (e.g., semaglutide), and the subject also receives a GLP-1 agonist treatment (e.g., semaglutide) during the administration.

In some embodiments, prior to the administering, the subject has not received prior treatment with a GLP-1 agonist (e.g., semaglutide), but the subject also receives a GLP-1 agonist treatment (e.g., semaglutide) during the administration.

In some embodiments, prior to the administering, the subject has received prior treatment with a GLP-1 agonist (e.g., semaglutide), but the subject does not receive a GLP-1 agonist treatment (e.g., semaglutide) during the administration.

In some embodiments, prior to the administering, the subject has not received prior treatment with a GLP-1 agonist (e.g., semaglutide), and the subject does not receive a GLP-1 agonist treatment (e.g., semaglutide) during the administration.

In some embodiments, prior to the administering, the subject is experiencing a weight-loss plateau. In some embodiments, prior to the administering, the subject is experiencing a weight-loss plateau while receiving treatment with a GLP-1 agonist (e.g., semaglutide).

In some embodiments, prior to the administering, the subject is experiencing a weight-loss plateau while receiving treatment with a GLP-1 agonist (e.g., semaglutide), and the subject also receives a GLP-1 agonist treatment (e.g., semaglutide) during the administration.

In some embodiments, prior to the administering, the subject is experiencing a weight-loss plateau while receiving treatment with a GLP-1 agonist (e.g., semaglutide), and the subject does not receive a GLP-1 agonist treatment (e.g., semaglutide) during the administration.

In some embodiments, prior to the administering, the subject has experienced a change in body weight of less than or equal to 2.5% (e.g., ≤2.4%, ≤2.3%, ≤2.2%, ≤2.1%, ≤2.0%, ≤1.9%, ≤1.8%, ≤1.7%, ≤1.6%, ≤1.5%, ≤1.4%, ≤1.3%, ≤1.2%, ≤1.1%, ≤1.0% 0.9%, ≤0.8%, ≤0.7%, ≤0.6%, ≤0.5%, 0.4%, ≤0.3% 0.2%, ≤0.1%; 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration. In some embodiments, the baseline is the subject's body weight 30-days prior to the administration. In some embodiments, the baseline is the subject's body weight prior to a first dose of a GLP-1 agonist (semaglutide).

In some embodiments, prior to the administering, the subject has experienced a change in body weight of less than or equal to 2.5% (e.g., ≤2.4%, ≤2.3%, ≤2.2%, ≤2.1%, ≤2.0%, ≤1.9%, ≤1.8%, ≤1.7%, ≤1.6%, ≤1.5%, ≤1.4%, ≤1.3%, ≤1.2%, ≤1.1%, ≤1.0% 0.9%, ≤0.8%, ≤0.7%, ≤0.6%, ≤0.5%, ≤0.4%, ≤0.3%, ≤0.2%, ≤0.1%; 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration while receiving treatment with a GLP-1 agonist (e.g., semaglutide). In some embodiments, the baseline is the subject's body weight 30-days prior to the administration. In some embodiments, the baseline is the subject's body weight prior to a first dose of a GLP-1 agonist.

In some embodiments, prior to the administering, the subject has experienced a change in body weight of less than or equal to 2.5% (e.g., ≤2.4%, ≤2.3%, ≤2.2%, ≤2.1%, ≤2.0%, ≤1.9%, ≤1.8%, ≤1.7%, ≤1.6%, ≤1.5%, ≤1.4%, ≤1.3%, ≤1.2%, ≤1.1%, ≤1.0% 0.9%, ≤0.8%, ≤0.7%, ≤0.6%, ≤0.5%, ≤0.4%, ≤0.3%, ≤0.2%, ≤0.1%; 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration while receiving treatment with a GLP-1 agonist (e.g., semaglutide), and the subject also receives a GLP-1 agonist treatment (e.g., semaglutide) during the administration.

In some embodiments, prior to the administering, the subject has experienced a change in body weight of less than or equal to 2.5% (e.g., ≤2.4%, ≤2.3%, ≤2.2%, ≤2.1%, ≤2.0%, ≤1.9%, ≤1.8%, ≤1.7%, ≤1.6%, ≤1.5%, ≤1.4%, ≤1.3%, ≤1.2%, ≤1.1%, ≤1.0%, ≤0.9%, ≤0.8%, ≤0.7%, ≤0.6%, ≤0.5%, ≤0.4%, ≤0.3%, ≤0.2%, ≤0.1%; 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration while receiving treatment with a GLP-1 agonist (e.g., semaglutide), and the subject does not receive a GLP-1 agonist treatment (e.g., semaglutide) during the administration.

In some embodiments, the subject is overweight or obese. In some embodiments, the subject is overweight. In some embodiments, the subject is obese.

In some embodiments, the subject has a body mass index (BMI) of at least 25.0 kg/m² (e.g., 25 kg/m², 25.5 kg/m², 26 kg/m², 26.5 kg/m², 27 kg/m², 27.5 kg/m², 28 kg/m², 28.5 kg/m², 29 kg/m², 29.5 kg/m², 30 kg/m², 30.5 kg/m², 31 kg/m², 31.5 kg/m², 32 kg/m², 32.5 kg/m², 33 kg/m², 33.5 kg/m², 34 kg/m², 34.5 kg/m², 35 kg/m², 35.5 kg/m², 36 kg/m², 36.5 kg/m², 37 kg/m², 37.5 kg/m², 38 kg/m², 38.5 kg/m², 39 kg/m², 39.5 kg/m², 40 kg/m²). In some embodiments, the subject has a body mass index (BMI) of at least 30.0 kg/m².

In some embodiments, the subject has a body mass index (BMI) in the range of 25.0 kg/m² to 29.9 kg/m², inclusive of the endpoints.

In some embodiments, the subject has a body mass index (BMI) in the range of 30.0 kg/m² to 40.0 kg/m², inclusive of the endpoints.

In some embodiments, the subject is a human subject.

Another aspect of the disclosure provides a polypeptide, molecule, or pharmaceutical composition disclosed herein for use in reducing body weight and/or food intake in a subject in need thereof. Still another aspect of the disclosure provides a use of a polypeptide, molecule, or pharmaceutical composition disclosed herein in the manufacture of a medicament for reducing body weight and/or food intake in a subject in need thereof.

Another aspect of the disclosure provides a GCGR agonist and a GIPR antagonist for use in therapy. Yet another aspect of the disclosure provides a GCGR agonist for use in therapy in combination with a GIPR antagonist. Still another aspect provides a GIPR antagonist for use in therapy in combination with a GCGR agonist. In some cases, the GCGR agonist and the GIPR antagonist may be used in acute therapy. In other cases, the GCGR agonist and the GIPR antagonist may be used in chronic therapy. The GCGR agonist and the GIPR antagonist may be present in the same or separate pharmaceutical compositions.

Another aspect of the disclosure provides a use of a GCGR agonist and a GIPR antagonist in the preparation of a medicament. Yet another aspect of the disclosure provides a use of a GCGR agonist in the preparation of a medicament for use in combination therapy with a GIPR antagonist. Still another aspect of the disclosure provides a use of a GIPR antagonist in the preparation of a medicament for use in combination therapy with a GCGR agonist.

Another aspect of the disclosure provides a method of treating obesity in a subject in need thereof, the method comprising administering a glucagon receptor (GCGR) agonist and a GIPR antagonist to the subject. Still another aspect of the disclosure provides a method of reducing body weight and/or food intake in a subject in need thereof, the method comprising administering a glucagon receptor (GCGR) agonist and a GIPR antagonist to the subject.

In some embodiments, therapeutically effective amounts of the GCGR agonist and the GIPR antagonist are combined prior to administration to the subject.

In some embodiments, the GCGR agonist is administered to the subject concurrently with the GIPR antagonist.

In some embodiments, therapeutically effective amounts of the GCGR agonist and the GIPR antagonist are administered to the subject sequentially. In some embodiments, the GCGR agonist is administered to the subject after the GIPR antagonist. In some embodiments, the GCGR agonist is administered to the subject before the GIPR antagonist.

In some embodiments, the therapeutically effective amounts of the GCGR agonist and the GIPR antagonist are synergistically effective amounts.

In some embodiments, prior to the administering, the subject received a prior therapy comprising a GLP-1 agonist, such as, e.g., semaglutide.

In some embodiments, prior to the administering, the subject received a prior therapy comprising a GCGR agonist. In some embodiments, prior to the administering, the subject received a prior therapy comprising a GIPR antagonist.

In some embodiments, prior to the administering, the subject has received prior treatment with a GLP-1 agonist (e.g., semaglutide). In other embodiments, prior to the administering, the subject has not received prior treatment with a GLP-1 agonist (e.g., semaglutide).

In some embodiments, the subject also receives a GLP-1 agonist treatment (e.g., semaglutide) during the administration.

In some embodiments, the subject does not receive a GLP-1 agonist treatment (e.g., semaglutide) during the administration.

In some embodiments, prior to the administering, the subject has received prior treatment with a GLP-1 agonist (e.g., semaglutide), and the subject also receives a GLP-1 agonist treatment (e.g., semaglutide) during the administration.

In some embodiments, prior to the administering, the subject has not received prior treatment with a GLP-1 agonist (e.g., semaglutide), but the subject also receives a GLP-1 agonist treatment (e.g., semaglutide) during the administration.

In some embodiments, prior to the administering, the subject has received prior treatment with a GLP-1 agonist (e.g., semaglutide), but the subject does not receive a GLP-1 agonist treatment (e.g., semaglutide) during the administration.

In some embodiments, prior to the administering, the subject has not received prior treatment with a GLP-1 agonist (e.g., semaglutide), and the subject does not receive a GLP-1 agonist treatment (e.g., semaglutide) during the administration.

In some embodiments, prior to the administering, the subject is experiencing a weight-loss plateau. In some embodiments, prior to the administering, the subject is experiencing a weight-loss plateau while receiving treatment with a GLP-1 agonist (e.g., semaglutide).

In some embodiments, prior to the administering, the subject is experiencing a weight-loss plateau while receiving treatment with a GLP-1 agonist (e.g., semaglutide), and the subject also receives a GLP-1 agonist treatment (e.g., semaglutide) during the administration.

In some embodiments, prior to the administering, the subject is experiencing a weight-loss plateau while receiving treatment with a GLP-1 agonist (e.g., semaglutide), and the subject does not receive a GLP-1 agonist treatment (e.g., semaglutide) during the administration.

In some embodiments, prior to the administering, the subject has experienced a change in body weight of less than or equal to 2.5% (e.g., ≤2.4%, ≤2.3%, ≤2.2%, ≤2.1%, ≤2.0%, ≤1.9%, ≤1.8%, ≤1.7%, ≤1.6%, ≤1.5%, ≤1.4%, ≤1.3%, ≤1.2%, ≤1.1%, ≤1.0% 0.9%, ≤0.8%, ≤0.7%, ≤0.6%, ≤0.5%, ≤0.4%, ≤0.3%, 0.2%, ≤0.1%; 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration. In some embodiments, the baseline is the subject's body weight 30-days prior to the administration. In some embodiments, the baseline is the subject's body weight prior to a first dose of a GLP-1 agonist (semaglutide).

In some embodiments, prior to the administering, the subject has experienced a change in body weight of less than or equal to 2.5% (e.g., ≤2.4%, ≤2.3%, ≤2.2%, ≤2.1%, ≤2.0%, ≤1.9%, ≤1.8%, ≤1.7%, ≤1.6%, ≤1.5%, ≤1.4%, ≤1.3%, ≤1.2%, ≤1.1%, ≤1.0%, ≤0.9%, ≤0.8%, ≤0.7%, ≤0.6%, ≤0.5%, ≤0.4%, ≤0.3%, ≤0.2%, ≤0.1%; 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration while receiving treatment with a GLP-1 agonist (e.g., semaglutide). In some embodiments, the baseline is the subject's body weight 30-days prior to the administration. In some embodiments, the baseline is the subject's body weight prior to a first dose of a GLP-1 agonist.

In some embodiments, prior to the administering, the subject has experienced a change in body weight of less than or equal to 2.5% (e.g., ≤2.4%, ≤2.3%, ≤2.2%, ≤2.1%, ≤2.0%, ≤1.9%, ≤1.8%, ≤1.7%, ≤1.6%, ≤1.5%, ≤1.4%, ≤1.3%, ≤1.2%, ≤1.1%, ≤1.0% 0.9%, ≤0.8%, ≤0.7%, ≤0.6%, ≤0.5%, ≤0.4%, ≤0.3%, ≤0.2%, ≤0.1%; 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration while receiving treatment with a GLP-1 agonist (e.g., semaglutide), and the subject also receives a GLP-1 agonist treatment (e.g., semaglutide) during the administration.

In some embodiments, prior to the administering, the subject has experienced a change in body weight of less than or equal to 2.5% (e.g., ≤2.4%, ≤2.3%, ≤2.2%, ≤2.1%, ≤2.0%, ≤1.9%, ≤1.8%, ≤1.7%, ≤1.6%, ≤1.5%, ≤1.4%, ≤1.3%, ≤1.2%, ≤1.1%, ≤1.0% 0.9%, 0.8%, ≤0.7%, ≤0.6%, ≤0.5%, ≤0.4%, ≤0.3%, 0.2% 0.1%; 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration while receiving treatment with a GLP-1 agonist (e.g., semaglutide), and the subject does not receive a GLP-1 agonist treatment (e.g., semaglutide) during the administration.

In some embodiments, the subject has a body mass index (BMI) of at least 30.0 kg/m² (e.g., 30 kg/m², 30.5 kg/m², 31 kg/m², 31.5 kg/m², 32 kg/m², 32.5 kg/m², 33 kg/m², 33.5 kg/m², 34 kg/m², 34.5 kg/m², 35 kg/m², 35.5 kg/m², 36 kg/m², 36.5 kg/m², 37 kg/m², 37.5 kg/m², 38 kg/m², 38.5 kg/m², 39 kg/m², 39.5 kg/m², 40 kg/m²).

In some embodiments, the subject has a body mass index (BMI) in the range of 30.0 kg/m² to 40.0 kg/m², inclusive of the endpoints.

In some embodiments, the subject is a human subject.

In some embodiments, the administration reduces body weight. In some embodiments, the administration reduces body weight gain. In some embodiments, the administration reduces fat mass.

In some embodiments, the administration lowers blood glucose, insulin, triglyceride, or cholesterol levels; reduces body weight; or improves glucose tolerance, energy expenditure, or insulin sensitivity. In some embodiments, the administration lowers blood glucose, insulin, triglyceride, or cholesterol levels. In some embodiments, the administration lowers blood glucose levels. In some embodiments, the administration lowers insulin levels. In some embodiments, the administration lowers triglyceride levels. In some embodiments, the administration lowers cholesterol levels. In some embodiments, the administration reduces body weight. In some embodiments, the administration improves glucose tolerance, energy expenditure, or insulin sensitivity. In some embodiments, the administration improves glucose tolerance. In some embodiments, the administration improves energy expenditure. In some embodiments, the administration improves insulin sensitivity.

In some embodiments, administration of the GCGR agonist in combination with the GIPR antagonist provides one or more sustained beneficial effect(s).

### Combination Therapies

Polypeptides and molecules disclosed herein can be co-administered with other active ingredients, if desired. The identity and properties of other active ingredients will depend on the nature of the condition to be treated or ameliorated.

Illustratively, the present disclosure provides methods for combination therapies in which a GLP-1 agonist (e.g., semaglutide) is used in combination with a polypeptide or molecule disclosed herein. In some cases, the polypeptides and molecules of the disclosure can be administered simultaneously, separately, or sequentially with an effective amount of a GLP-1 agonist (e.g., semaglutide) in any of the methods described herein. In some embodiments, the GLP-1 agonist (e.g., semaglutide) is administered as a pharmaceutical composition comprising the GLP-1 agonist (e.g., semaglutide) and a pharmaceutically acceptable excipient.

For example, provided herein is a method of treating obesity in a subject in need thereof, the method comprising administering a polypeptide, molecule, or pharmaceutical composition disclosed herein to the subject in combination with a GLP-1 agonist (e.g., semaglutide).

In some embodiments, the subject is overweight or obese. In some embodiments, the subject is overweight. In some embodiments, the subject is obese.

In some embodiments, the subject has a body mass index (BMI) of at least 25.0 kg/m² (e.g., 25 kg/m², 25.5 kg/m², 26 kg/m², 26.5 kg/m², 27 kg/m², 27.5 kg/m², 28 kg/m², 28.5 kg/m², 29 kg/m², 29.5 kg/m², 30 kg/m², 30.5 kg/m², 31 kg/m², 31.5 kg/m², 32 kg/m², 32.5 kg/m², 33 kg/m², 33.5 kg/m², 34 kg/m², 34.5 kg/m², 35 kg/m², 35.5 kg/m², 36 kg/m², 36.5 kg/m², 37 kg/m², 37.5 kg/m², 38 kg/m², 38.5 kg/m², 39 kg/m², 39.5 kg/m², 40 kg/m²). In some embodiments, the subject has a body mass index (BMI) of at least 30.0 kg/m².

In some embodiments, the subject has a body mass index (BMI) in the range of 25.0 kg/m² to 29.9 kg/m², inclusive of the endpoints.

In some embodiments, the subject has a body mass index (BMI) in the range of 30.0 kg/m² to 40.0 kg/m², inclusive of the endpoints.

In some embodiments, the subject is a human subject.

In some embodiments, the GLP-1 agonist (e.g., semaglutide) and the polypeptide, molecule, or pharmaceutical composition are administered in consecutive, non-overlapping dosing intervals, wherein the GLP-1 agonist (e.g., semaglutide) is administered prior to the polypeptide, molecule, or pharmaceutical composition.

In some embodiments, the GLP-1 agonist (e.g., semaglutide) and the polypeptide, molecule, or pharmaceutical composition are administered concurrently.

In some embodiments, at least one dose of the GLP-1 agonist (e.g., semaglutide) is administered prior to a first administration of the polypeptide, molecule, or pharmaceutical composition.

In some embodiments, prior to the administering, the subject has received prior treatment with a GLP-1 agonist (e.g., semaglutide). In other embodiments, prior to the administering, the subject has not received prior treatment with a GLP-1 agonist (e.g., semaglutide).

In some embodiments, prior to the administering, the subject is experiencing a weight-loss plateau. In some embodiments, prior to the administering, the subject is experiencing a weight-loss plateau while receiving treatment with a GLP-1 agonist (e.g., semaglutide).

In some embodiments, prior to the administering, the subject has experienced a change in body weight of less than or equal to 2.5% (e.g., ≤2.4%, ≤2.3%, ≤2.2%, ≤2.1%, ≤2.0%, ≤1.9%, ≤1.8%, ≤1.7%, ≤1.6%, ≤1.5%, ≤1.4%, ≤1.3%, ≤1.2%, ≤1.1%, ≤1.0%, ≤0.9%, ≤0.8%, ≤0.7%, ≤0.6%, ≤0.5%, 0.4% 0.3% 0.2%, ≤0.1%; 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration. In some embodiments, the baseline is the subject's body weight 30-days prior to the administration. In some embodiments, the baseline is the subject's body weight prior to a first dose of a GLP-1 agonist (semaglutide).

Also provided herein is a method of reducing body weight and/or food intake in a subject in need thereof, the method comprising administering a polypeptide, molecule, or pharmaceutical composition disclosed herein to the subject in combination with a GLP-1 agonist (e.g., semaglutide).

In some embodiments, the subject is overweight or obese. In some embodiments, the subject is overweight. In some embodiments, the subject is obese.

In some embodiments, the subject has a body mass index (BMI) of at least 25.0 kg/m² (e.g., 25 kg/m², 25.5 kg/m², 26 kg/m², 26.5 kg/m², 27 kg/m², 27.5 kg/m², 28 kg/m², 28.5 kg/m², 29 kg/m², 29.5 kg/m², 30 kg/m², 30.5 kg/m², 31 kg/m², 31.5 kg/m², 32 kg/m², 32.5 kg/m², 33 kg/m², 33.5 kg/m², 34 kg/m², 34.5 kg/m², 35 kg/m², 35.5 kg/m², 36 kg/m², 36.5 kg/m², 37 kg/m², 37.5 kg/m², 38 kg/m², 38.5 kg/m², 39 kg/m², 39.5 kg/m², 40 kg/m²). In some embodiments, the subject has a body mass index (BMI) of at least 30.0 kg/m².

In some embodiments, the subject has a body mass index (BMI) in the range of 25.0 kg/m² to 29.9 kg/m², inclusive of the endpoints.

In some embodiments, the subject has a body mass index (BMI) in the range of 30.0 kg/m² to 40.0 kg/m², inclusive of the endpoints.

In some embodiments, the subject is a human subject.

In some embodiments, the GLP-1 agonist (e.g., semaglutide) and the polypeptide, molecule, or pharmaceutical composition are administered in consecutive, non-overlapping dosing intervals, wherein the GLP-1 agonist (e.g., semaglutide) is administered prior to the polypeptide, molecule, or pharmaceutical composition.

In some embodiments, the GLP-1 agonist (e.g., semaglutide) and the polypeptide, molecule, or pharmaceutical composition are administered concurrently.

In some embodiments, at least one dose of the GLP-1 agonist (e.g., semaglutide) is administered prior to a first administration of the polypeptide, molecule, or pharmaceutical composition.

In some embodiments, prior to the administering, the subject has received prior treatment with a GLP-1 agonist (e.g., semaglutide). In other embodiments, prior to the administering, the subject has not received prior treatment with a GLP-1 agonist (e.g., semaglutide).

In some embodiments, prior to the administering, the subject is experiencing a weight-loss plateau. In some embodiments, prior to the administering, the subject is experiencing a weight-loss plateau while receiving treatment with a GLP-1 agonist (e.g., semaglutide).

In some embodiments, prior to the administering, the subject has experienced a change in body weight of less than or equal to 2.5% (e.g., ≤2.4%, ≤2.3%, ≤2.2%, ≤2.1%, ≤2.0%, 1.9%, ≤1.8%, ≤1.7%, ≤1.6%, ≤1.5%, ≤1.4%, ≤1.3%, ≤1.2%, ≤1.1%, ≤1.0% 0.9%, ≤0.8%, ≤0.7%, ≤0.6%, ≤0.5%, ≤0.4%, ≤0.3% 0.2%, ≤0.1%; 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0%) relative to a baseline in the 30-days preceding the administration. In some embodiments, the baseline is the subject's body weight 30-days prior to the administration. In some embodiments, the baseline is the subject's body weight prior to a first dose of a GLP-1 agonist (semaglutide).

Provided herein is a GLP-1 agonist (e.g., semaglutide) and a polypeptide or molecule disclosed herein for use in treating obesity. Also provided herein is a GLP-1 agonist for use in treating obesity in combination with a polypeptide or molecule disclosed herein. Further provided herein is a polypeptide or molecule disclosed herein for use in treating obesity in combination with a GLP-1 agonist (e.g., semaglutide).

Provided herein is a GLP-1 agonist (e.g., semaglutide) and a polypeptide or molecule disclosed herein for use in reducing body weight and/or food intake in a subject in need thereof. Also provided herein is a GLP-1 agonist for use in reducing body weight and/or food intake in a subject in need thereof in combination with a polypeptide or molecule disclosed herein. Further provided herein is a polypeptide or molecule disclosed herein for use in reducing body weight and/or food intake in a subject in need thereof in combination with a GLP-1 agonist (e.g., semaglutide).

Provided herein is a use of a GLP-1 agonist (e.g., semaglutide) and a polypeptide or molecule disclosed herein for the manufacture of a medicament for treating obesity. Also provided herein is a use of a GLP-1 agonist (e.g., semaglutide) for the manufacture of a medicament for treating obesity in combination with a polypeptide or molecule disclosed herein. Further provided herein is a use of a polypeptide or a molecule disclosed herein for the manufacture of a medicament for treating obesity in combination with a GLP-1 agonist (e.g., semaglutide).

Provided herein is a use of a GLP-1 agonist (e.g., semaglutide) and a polypeptide or molecule disclosed herein for the manufacture of a medicament for reducing body weight and/or food intake in a subject in need thereof. Also provided herein is a use of a GLP-1 agonist (e.g., semaglutide) for the manufacture of a medicament for reducing body weight and/or food intake in a subject in need thereof in combination with a polypeptide or molecule disclosed herein. Further provided herein is a use of a polypeptide or a molecule disclosed herein for the manufacture of a medicament for reducing body weight and/or food intake in a subject in need thereof in combination with a GLP-1 agonist (e.g., semaglutide).

### Kits

Also provided herein are kits for practicing the disclosed methods. Such kits can comprise a pharmaceutical composition, such as those described herein, which can be provided in a sterile container. Optionally, instructions on how to employ the provided pharmaceutical composition in weight management and/or the treatment of obesity can also be included or be made available to a patient or a medical service provider.

In one aspect, a kit comprises (a) a pharmaceutical composition disclosed herein; and (b) one or more containers for the pharmaceutical composition. Such a kit can also comprise instructions for the use thereof; the instructions can be tailored to the patient population being treated. In some cases, the instructions can describe the use and nature of the materials provided in the kit. For example, in some embodiments, the kit comprises instructions for a patient to carry out administration.

The optional instructions can be printed on a substrate, such as paper or plastic, etc., and can be present in a kit as a package insert, in the labeling of the container of the kit or components thereof (e.g., associated with the packaging), etc. In some embodiments, the instructions are present as an electronic storage data file present on a suitable computer readable storage medium, such as, e.g., a CD-ROM, diskette, etc. In other embodiments, the actual instructions are not present in the kit, but means for obtaining the instructions from a remote source, such as over the internet, are provided. Illustratively, in some embodiments, the kit includes a web address where the instructions can be viewed and/or from which the instructions can be downloaded.

Often it will be desirable that some or all components of a kit are packaged in suitable packaging to maintain sterility. In some embodiments, the components of a kit can be packaged in a kit containment element to make a single, easily handled unit, where the kit containment element, e.g., box or analogous structure, may or may not be an airtight container, e.g., to further preserve the sterility of some or all of the components of the kit.

### Additional Non-Limiting Example Embodiments

Non-limiting example embodiments of the present disclosure also include:

E1. A polypeptide that agonizes a glucagon receptor (“GCGR”), wherein:
the polypeptide comprises at least 25 amino acids, wherein the polypeptide comprises 5-bromo-tryptophan at position 25; and
the polypeptide has at least 79% sequence identity to the amino acid sequence of SEQ ID NO: 1587.

E2. The polypeptide of E1, wherein the polypeptide has at least 82% sequence identity to the amino acid sequence of SEQ ID NO: 1587.

E3. The polypeptide of E1 or E2, wherein the polypeptide has at least 86% sequence identity to the amino acid sequence of SEQ ID NO: 1587.

E4. The polypeptide of any one of E1 to E3, wherein the polypeptide has at least 89% sequence identity to the amino acid sequence of SEQ ID NO: 1587.

E5. The polypeptide of any one of E1 to E4, wherein the polypeptide has at least 93% sequence identity to the amino acid sequence of SEQ ID NO: 1587.

E6. The polypeptide of any one of E1 to E5, wherein the polypeptide has at least 96% sequence identity to the amino acid sequence of SEQ ID NO: 1587.

E7. The polypeptide of E1, wherein the polypeptide comprises the amino acid sequence of any one of SEQ ID NOs: 1587-1595.

E8. The polypeptide of E1, wherein the polypeptide comprises the amino acid sequence of SEQ ID NO: 1587.

E9. The polypeptide of E1, wherein the polypeptide consists of the amino acid sequence of SEQ ID NO: 1587.

E10. The polypeptide of E1, wherein the polypeptide comprises the amino acid sequence of SEQ ID NO: 1588.

E11. The polypeptide of E1, wherein the polypeptide consists of the amino acid sequence of SEQ ID NO: 1588.

E12. The polypeptide of E1, wherein the polypeptide comprises the amino acid sequence of SEQ ID NO: 1589.

E13. The polypeptide of E1, wherein the polypeptide consists of the amino acid sequence of SEQ ID NO: 1589.

E14. The polypeptide of E1, wherein the polypeptide comprises the amino acid sequence of SEQ ID NO: 1590.

E15. The polypeptide of E1, wherein the polypeptide consists of the amino acid sequence of SEQ ID NO: 1590.

E16. The polypeptide of E1, wherein the polypeptide comprises the amino acid sequence of SEQ ID NO: 1591.

E17. The polypeptide of E1, wherein the polypeptide consists of the amino acid sequence of SEQ ID NO: 1591.

E18. The polypeptide of E1, wherein the polypeptide comprises the amino acid sequence of SEQ ID NO: 1592.

E19. The polypeptide of E1, wherein the polypeptide consists of the amino acid sequence of SEQ ID NO: 1592.

E20. The polypeptide of E1, wherein the polypeptide comprises the amino acid sequence of SEQ ID NO: 1593.

E21. The polypeptide of E1, wherein the polypeptide consists of the amino acid sequence of SEQ ID NO: 1593.

E22. The polypeptide of E1, wherein the polypeptide comprises the amino acid sequence of SEQ ID NO: 1594.

E23. The polypeptide of E1, wherein the polypeptide consists of the amino acid sequence of SEQ ID NO: 1594.

E24. The polypeptide of E1, wherein the polypeptide comprises the amino acid sequence of SEQ ID NO: 1595.

E25. The polypeptide of E1, wherein the polypeptide consists of the amino acid sequence of SEQ ID NO: 1595.

E26. The polypeptide of E1, comprising at least 27 amino acids.

E27. The polypeptide of E26, wherein the polypeptide further comprises one or more of:
d-serine at position 2;
2-aminoisobutyric acid at position 16;
lysine at position 17;
β-(2-naphthyl)-L-alanine at position 18;
aspartic acid, lysine, alanine, or glutamic acid at position 24; and
leucine at position 27.

E28. The polypeptide of any one of E1, E26, or E27, wherein the polypeptide further comprises d-serine at position 2.

E29. The polypeptide of any one of E1 or E26-E28, wherein the polypeptide further comprises 2-aminoisobutyric acid at position 16.

E30. The polypeptide of any one of E1 or E26-E29, wherein the polypeptide further comprises lysine at position 17.

E31. The polypeptide of any one of E1 or E26-E30, wherein the polypeptide further comprises β-(2-naphthyl)-L-alanine at position 18.

E32. The polypeptide of any one of E1 or E26-E31, wherein the polypeptide further comprises aspartic acid at position 24.

E33. The polypeptide of any one of E1 or E26-E31, wherein the polypeptide further comprises lysine at position 24.

E34. The polypeptide of any one of E1 or E26-E31, wherein the polypeptide further comprises alanine at position 24.

E35. The polypeptide of any one of E1 or E26-E31, wherein the polypeptide further comprises glutamic acid at position 24.

E36. The polypeptide of any one of E1 or E26-E35, wherein the polypeptide further comprises leucine at position 27.

E37. The polypeptide of any one of E1 or E26-E36, wherein the polypeptide comprises d-serine at position 2 and 2-aminoisobutyric acid at position 16.

E38. The polypeptide of any one of E1 or E26-E37, wherein the polypeptide comprises d-serine at position 2, 2-aminoisobutyric acid at position 16, and leucine at position 27.

E39. The polypeptide of any one of E1 or E26-E38, comprising at least 28 amino acids.

E40. The polypeptide of E36, wherein the polypeptide further comprises lysine or aspartic acid at position 28.

E41. The polypeptide of E36 or E37, wherein the polypeptide further comprises lysine at position 28.

E42. The polypeptide of E36 or E37, wherein the polypeptide further comprises aspartic acid at position 28.

E43. The polypeptide of E1 or E39, wherein the polypeptide further comprises d-serine at position 2, 2-aminoisobutyric acid at position 16, leucine at position 27, and lysine or aspartic acid at position 28.

E44. The polypeptide of E1 or E39, wherein the polypeptide further comprises d-serine at position 2, 2-aminoisobutyric acid at position 16, aspartic acid at position 24, leucine at position 27, and lysine at position 28.

E45. The polypeptide of E1 or E39, wherein the polypeptide further comprises d-serine at position 2, 2-aminoisobutyric acid at position 16, leucine at position 27, and lysine at position 28.

E46. The polypeptide of E1 or E39, wherein the polypeptide further comprises d-serine at position 2, 2-aminoisobutyric acid at position 16, lysine at position 24, leucine at position 27, and aspartic acid at position 28.

E47. The polypeptide of E1 or E39, wherein the polypeptide further comprises d-serine at position 2, 2-aminoisobutyric acid at position 16, aspartic acid at position 24, leucine at position 27, and lysine at position 28.

E48. The polypeptide of E1 or E39, wherein the polypeptide further comprises d-serine at position 2, 2-aminoisobutyric acid at position 16, lysine at position 17, alanine at position 24, leucine at position 27, and lysine at position 28.

E49. The polypeptide of E1 or E39, wherein the polypeptide further comprises d-serine at position 2, 2-aminoisobutyric acid at position 16, alanine at position 24, and lysine at position 28.

E50. The polypeptide of E1 or E39, wherein the polypeptide further comprises d-serine at position 2, 2-aminoisobutyric acid at position 16, lysine at position 17, glutamic acid at position 24, leucine at position 27, and lysine at position 28.

E51. The polypeptide of E1 or E39, wherein the polypeptide further comprises d-serine at position 2, 2-aminoisobutyric acid at position 16, β-(2-naphthyl)-L-alanine at position 18, lysine at position 24, leucine at position 27, and aspartic acid at position 28.

E52. The polypeptide of E1 or E39, wherein the polypeptide further comprises d-serine at position 2, 2-aminoisobutyric acid at position 16, β-(2-naphthyl)-L-alanine at position 18, alanine at position 24, leucine at position 27, and lysine at position 28.

E53. A polypeptide that agonizes a glucagon receptor (“GCGR”), wherein:
the polypeptide comprises at least 28 amino acids, wherein the polypeptide comprises 2-aminoisobutyric acid at position 16 and lysine, serine, or aspartic acid at position 28; and
the polypeptide has at least 89% sequence identity to the amino acid sequence of SEQ ID NO: 1596.

E54. The polypeptide of E53, wherein the polypeptide has at least 93% sequence identity to the amino acid sequence of SEQ ID NO: 1596.

E55. The polypeptide of E53, wherein the polypeptide has at least 96% sequence identity to the amino acid sequence of SEQ ID NO: 1596.

E56. The polypeptide of any one of E53-E56, wherein the polypeptide comprises lysine at position 28.

E57. The polypeptide of any one of E53-E56, wherein the polypeptide comprises serine at position 28.

E58. The polypeptide of any one of E53-E56, wherein the polypeptide comprises aspartic acid at position 28.

E59. The polypeptide of any one of E53-E58, wherein the polypeptide further comprises one or more of:
tyrosine at position 1;
d-serine, d-threonine, or 2-aminoisobutyric acid at position 2;
histidine at position 7;
lysine, citrulline, or glutamine at position 17;
β-(2-naphthyl)-L-alanine or β-(4,4′-biphenyl)alanine at position 18;
lysine, alanine, asparagine, glutamic acid, glycine, aspartic acid, histidine, threonine, or 2-aminoisobutyric acid at position 24;
tyrosine, 5-bromo-tryptophan, or L-beta-homotryptophan at position 25; and
leucine, glutamic acid, or α-aminoadipic acid at position 27.

E60. The polypeptide of E59, wherein the polypeptide further comprises d-serine, d-threonine, or 2-aminoisobutyric acid at position 2.

E61. The polypeptide of E59 or E60, wherein the polypeptide further comprises d-serine at position 2.

E62. The polypeptide of any one of E59-E61, wherein the polypeptide further comprises leucine, glutamic acid, or α-aminoadipic acid at position 27.

E63. The polypeptide of any one of E59-E62, wherein the polypeptide further comprises leucine at position 27.

E64. The polypeptide of any one of E53-E55, wherein the polypeptide comprises d-serine at position 2, 2-aminoisobutyric acid at position 16, leucine at position 27, and lysine at position 28.

E65. The polypeptide of any one of E53-E64, wherein the polypeptide further comprises lysine, citrulline, or glutamine at position 17.

E66. The polypeptide of E65, wherein the polypeptide further comprises lysine at position 17.

E67. The polypeptide of any one of E53-E55, wherein the polypeptide comprises d-serine at position 2, 2-aminoisobutyric acid at position 16, lysine at position 17, leucine at position 27, and lysine at position 28.

E68. The polypeptide of any one of E53-E64, wherein the polypeptide further comprises (3-(2-naphthyl)-L-alanine or β-(4,4′-biphenyl)alanine at position 18.

E69. The polypeptide of any one of E53-E64, wherein the polypeptide further comprises lysine, alanine, asparagine, glutamic acid, glycine, aspartic acid, histidine, threonine, or 2-aminoisobutyric acid at position 24.

E70. The polypeptide of E69, wherein the polypeptide further comprises lysine at position 24.

E71. The polypeptide of any one of E53-E55, wherein the polypeptide comprises d-serine at position 2, 2-aminoisobutyric acid at position 16, lysine, alanine, asparagine, glutamic acid, glycine, aspartic acid, histidine, threonine, or 2-aminoisobutyric acid at position 24, and lysine at position 28.

E72. The polypeptide of any one of E53-E55, wherein the polypeptide further comprises histidine at position 7.

E73. The polypeptide of any one of E53-E55, wherein the polypeptide further comprises tyrosine at position 1.

E74. The polypeptide of any one of E53-E55, wherein the polypeptide further comprises tyrosine, 5-bromo-tryptophan, or L-beta-homotryptophan at position 25.

E75. The polypeptide of any one of E53-E55, wherein the polypeptide comprises d-serine at position 2, 2-aminoisobutyric acid at position 16, tyrosine, 5-bromo-tryptophan, or L-beta-homotryptophan at position 25, leucine at position 27, and lysine at position 28.

E76. The polypeptide of any one of E53-E75, comprising at least 29 amino acids.

E77. The polypeptide of E76, wherein the polypeptide further comprises alanine, aspartic acid, glutamic acid, or serine at position 29.

E78. The polypeptide of E77, wherein the polypeptide comprises d-serine at position 2, 2-aminoisobutyric acid at position 16, lysine at position 28, and alanine, aspartic acid, glutamic acid, or serine at position 29.

E79. A polypeptide that agonizes a glucagon receptor (“GCGR”), wherein:
the polypeptide comprises at least 28 amino acids, wherein the polypeptide comprises 2-aminoisobutyric acid at position 16, leucine at position 27, and lysine at position 28; and
the polypeptide has at least 89% sequence identity to the amino acid sequence of SEQ ID NO: 1615.

E80. The polypeptide of E79, wherein the polypeptide has at least 93% sequence identity to the amino acid sequence of SEQ ID NO: 1615.

E81. The polypeptide of E79, wherein the polypeptide has at least 96% sequence identity to the amino acid sequence of SEQ ID NO: 1615.

E82. The polypeptide of any one of E79-E81, wherein the polypeptide further comprises d-serine at position 2.

E83. The polypeptide of any one of E79-E82, wherein the polypeptide further comprises aspartic acid or glutamic acid at position 24.

E84. The polypeptide of any one of E79-E83, wherein the polypeptide further comprises d-serine at position 2 and aspartic acid or glutamic acid at position 24.

E85. The polypeptide of any one of E79-E84, wherein the polypeptide further comprises d-serine at position 2 and aspartic acid at position 24.

E86. The polypeptide of any one of E79-E85, wherein the polypeptide further comprises lysine at position 17.

E87. The polypeptide of any one of E79-E86, wherein the polypeptide further comprises d-serine at position 2, lysine at position 17, and aspartic acid at position 24.

E88. A polypeptide that agonizes a glucagon receptor (“GCGR”), wherein:
the polypeptide comprises at least 28 amino acids, wherein the polypeptide comprises 2-aminoisobutyric acid at position 16, aspartic acid at position 24, and lysine at position 28; and
the polypeptide has at least 89% sequence identity to the amino acid sequence of SEQ ID NO: 1626.

E89. The polypeptide of E88, wherein the polypeptide has at least 93% sequence identity to the amino acid sequence of SEQ ID NO: 1626.

E90. The polypeptide of E88, wherein the polypeptide has at least 96% sequence identity to the amino acid sequence of SEQ ID NO: 1626.

E91. The polypeptide of any one of E88-E90, wherein the polypeptide further comprises d-serine at position 2.

E92. The polypeptide of any one of E88-E91, wherein the polypeptide further comprises lysine at position 17.

E93. The polypeptide of any one of E88-E92, wherein the polypeptide further comprises leucine at position 27.

E94. A polypeptide that agonizes a glucagon receptor (“GCGR”) comprising the amino acid sequence of any one of SEQ ID NOs: 1587-1627.

E95. The polypeptide of E94, wherein the polypeptide comprises the amino acid sequence of SEQ ID NO: 1596.

E96. The polypeptide of E94, wherein the polypeptide comprises the amino acid sequence of SEQ ID NO: 1597.

E97. The polypeptide of E94, wherein the polypeptide comprises the amino acid sequence of SEQ ID NO: 1615.

E98. The polypeptide of E94, wherein the polypeptide comprises the amino acid sequence of SEQ ID NO: 1626.

E99. The polypeptide of E94, wherein the polypeptide consists of the amino acid sequence of any one of SEQ ID NOs: 1587-1627.

E100. The polypeptide of E99, wherein the polypeptide consists of the amino acid sequence of SEQ ID NO: 1596.

E101. The polypeptide of E99, wherein the polypeptide consists of the amino acid sequence of SEQ ID NO: 1597.

E102. The polypeptide of E99, wherein the polypeptide consists of the amino acid sequence of SEQ ID NO: 1615.

E103. The polypeptide of E99, wherein the polypeptide consists of the amino acid sequence of SEQ ID NO: 1626.

E104. The polypeptide of any one of E1-E103, wherein the polypeptide agonizes human GCGR (“hGCGR”).

E105. The polypeptide of any one of E1-E104, wherein the polypeptide has a hGCGR EC₅₀ of less than or equal to 500 pM.

E106. The polypeptide of any one of E1-E105, wherein the polypeptide has a human glucagon-like peptide-1 receptor (“hGLP-1R”) EC₅₀:hGCGR EC₅₀ ratio of at least 40:1 (e.g., at least 50:1, at least 60:1, at least 70:1, at least 80:1, at least 90:1, at least 100:1, at least 250:1, at least 500:1, at least 1000:1).

E107. The polypeptide of any one of E1-E106, wherein the polypeptide has a hGLP-1R EC₅₀:hGCGR EC₅₀ ratio of at least 100:1.

E108. The polypeptide of any one of E1-E107, wherein the polypeptide has a hGLP-1R EC₅₀:hGCGR EC₅₀ ratio of at least 500:1.

E109. A molecule comprising:
a first polypeptide that agonizes a glucagon receptor (“GCGR”), wherein the first polypeptide is selected from the polypeptides of any one of E1-108; and
a second polypeptide comprising the amino acid sequence of any one of SEQ ID NOs: 1628-1683,
wherein the C-terminus of the second polypeptide is covalently linked to an ε-amino group of a lysine residue of the first polypeptide (e.g., an ε-amino group of a lysine residue of the first polypeptide has been condensed with a carboxyl group of the C-terminus of the second polypeptide to form an amide bond between the first polypeptide and the second polypeptide).

E110. The molecule of E109, wherein the C-terminus of the second polypeptide is covalently linked to the ε-amino group of a lysine at position 24 or position 28 of the first polypeptide (e.g., the ε-amino group of a lysine residue at position 24 or position 28 of the first polypeptide has been condensed with a carboxyl group of the C-terminus of the second polypeptide to form an amide bond between the first polypeptide and the second polypeptide).

E111. The molecule of E109 or E110, wherein the C-terminus of the second polypeptide is covalently linked to the ε-amino group of a lysine at position 24 of the first polypeptide.

E112. The molecule of E109 or E110, wherein the C-terminus of the second polypeptide is covalently linked to the ε-amino group of a lysine at position 28 of the first polypeptide.

E113. The molecule of any one of E109-E112, wherein the second polypeptide comprises the amino acid sequence of any one of SEQ ID NOs: 1628-1630.

E114. The molecule of any one of E109-E113, wherein the second polypeptide comprises the amino acid sequence of SEQ ID NO: 1628.

E115. The molecule of any one of E109-E113, wherein the second polypeptide comprises the amino acid sequence of SEQ ID NO: 1629.

E116. The molecule of any one of E109-E113, wherein the second polypeptide comprises the amino acid sequence of SEQ ID NO: 1630.

E117. The molecule of any one of E109-E113, wherein the first polypeptide comprises the amino acid sequence of any one of SEQ ID NOs: 1587-1627.

E118. The molecule of any one of E109-E117, wherein the first polypeptide comprises the amino acid sequence of any one of SEQ ID NOs: 1587, 1592, 1596, 1615, or 1626.

E119. The molecule of any one of E109-E118, wherein the first polypeptide comprises the amino acid sequence of SEQ ID NO: 1587.

E120. The molecule of any one of E109-E118, wherein the first polypeptide comprises the amino acid sequence of SEQ ID NO: 1592.

E121. The molecule of any one of E109-E118, wherein the first polypeptide comprises the amino acid sequence of SEQ ID NO: 1596.

E122. The molecule of any one of E109-E118, wherein the first polypeptide comprises the amino acid sequence of SEQ ID NO: 1615.

E123. The molecule of any one of E109-E118, wherein the first polypeptide comprises the amino acid sequence of SEQ ID NO: 1626.

E124. A molecule comprising:
a polypeptide that agonizes a glucagon receptor (“GCGR”), wherein the polypeptide is selected from the polypeptides of any one of E1-E108; and
a half-life extending domain (e.g., an Fc-containing polypeptide).

E125. The molecule of E124, wherein:
the half-life extending domain is an Fc-containing polypeptide;
   an ε-amino group of a lysine residue of the polypeptide is covalently linked to a C-terminus of a linker polypeptide; and
   an N-terminus of the linker polypeptide is conjugated to a cysteine residue of the Fc-containing polypeptide.

E126. The molecule of E125, wherein the linker polypeptide is a linear polypeptide.

E127. The molecule of E125 or E126, wherein the linker polypeptide comprises the amino acid sequence of any one of SEQ ID NOs: 1628-1683.

E128. The molecule of any one of E125-E127, wherein the linker polypeptide comprises the amino acid sequence of any one of SEQ ID NOs: 1628-1630.

E129. The molecule of any one of E125-E128, wherein the linker polypeptide comprises the amino acid sequence of SEQ ID NO: 1628.

E130. The molecule of any one of E125-E128, wherein the linker polypeptide comprises the amino acid sequence of SEQ ID NO: 1629.

E131. The molecule of any one of E125-E128, wherein the linker polypeptide comprises the amino acid sequence of SEQ ID NO: 1630.

E132. The molecule of any one of E124-E131, wherein the polypeptide comprises the amino acid sequence of any one of SEQ ID NOs: 1587-1627.

E133. The molecule of any one of E124-E132, wherein the polypeptide comprises the amino acid sequence of any one of SEQ ID NOs: 1588 or 1596-1611.

E134. The molecule of any one of E124-E132, wherein the polypeptide comprises the amino acid sequence of any one of SEQ ID NOs: 1587, 1592, 1596, 1615, or 1626.

E135. The molecule of any one of E124-E132, wherein the polypeptide comprises the amino acid sequence of SEQ ID NO: 1587.

E136. The molecule of any one of E124-E132, wherein the polypeptide comprises the amino acid sequence of SEQ ID NO: 1592.

E137. The molecule of any one of E124-E132, wherein the polypeptide comprises the amino acid sequence of SEQ ID NO: 1596.

E138. The molecule of any one of E124-E132, wherein the polypeptide comprises the amino acid sequence of SEQ ID NO: 1615.

E139. The molecule of any one of E124-E132, wherein the polypeptide comprises the amino acid sequence of SEQ ID NO: 1626.

E140. The molecule of any one of E124-E139, wherein the half-life extending domain is an antibody fragment.

E141. The molecule of any one of E124-E139, wherein the half-life extending domain is an antibody.

E142. The molecule of E140, wherein the antibody is of the IgG1-, IgG2-, or IgG4-subclass.

E143. The molecule of E140 or E141, wherein the antibody is of the IgG1- or IgG2-subclass.

E144. The molecule of any one of E141-E143, wherein the antibody specifically binds to 2,4-dinitrophenol (“DNP”).

E145. The molecule of any one of E141-E143, wherein the antibody specifically binds to a glucose-dependent insulinotropic polypeptide receptor (“GIPR”).

E146. The molecule of any one of E141-E143, wherein the antibody specifically binds to human GIPR.

E147. The molecule of E145 or E146, wherein the antibody inhibits binding of GIP to the extracellular portion of human GIPR.

E148. The molecule of E147, wherein the antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise the amino acid sequences of SEQ ID NO: 702, SEQ ID NO: 859, SEQ ID NO: 1016, SEQ ID NO: 1173, SEQ ID NO: 1330, and SEQ ID NO: 1487, respectively.

E149. The molecule of E147 or E148, wherein the antibody comprises a light chain variable region comprising the amino acid sequence of SEQ ID NO: 74 and a heavy chain variable region comprising the amino acid sequence of SEQ ID NO: 231.

E150. The molecule of any one of E145-E149, wherein the antibody comprises a light chain comprising the amino acid sequence of SEQ ID NO: 388 and a heavy chain comprising the amino acid sequence of SEQ ID NO: 1571.

E151. A molecule comprising:
a first polypeptide that agonizes a glucagon receptor (“GCGR”); and
an antibody that specifically binds to a glucose-dependent insulinotropic polypeptide receptor (“GIPR”).

E152. The molecule of E151, wherein:
an ε-amino group of a lysine residue of the first polypeptide is covalently linked to a C-terminus of a first linker polypeptide; and
an N-terminus of the first linker polypeptide is conjugated to a cysteine residue of the antibody.

E153. The molecule of E151 or E152, wherein the first polypeptide is selected from the polypeptides of any one of E1-108.

E154. The molecule of any one of E151-E153, wherein the first polypeptide comprises the amino acid sequence of any one of SEQ ID NOs: 1587-1627.

E155. The molecule of any one of E151-E154, wherein the first polypeptide comprises the amino acid sequence of any one of SEQ ID NOs: 1587, 1592, 1596, 1615, or 1626.

E156. The molecule of any one of E151-E155, wherein the first polypeptide comprises the amino acid sequence of SEQ ID NO: 1587.

E157. The molecule of any one of E151-E155, wherein the first polypeptide comprises the amino acid sequence of SEQ ID NO: 1592.

E158. The molecule of any one of E151-E155, wherein the first polypeptide comprises the amino acid sequence of SEQ ID NO: 1596.

E159. The molecule of any one of E151-E155, wherein the first polypeptide comprises the amino acid sequence of SEQ ID NO: 1615.

E160. The molecule of any one of E151-E155, wherein the first polypeptide comprises the amino acid sequence of SEQ ID NO: 1626.

E161. The molecule of any one of E152-E160, wherein the first linker polypeptide is a linear polypeptide.

E162. The molecule of any one of E152-E161, wherein the first linker polypeptide comprises the amino acid sequence of any one of SEQ ID NOs: 1628-1683.

E163. The molecule of any one of E152-E162, wherein the first linker polypeptide comprises the amino acid sequence of any one of SEQ ID NOs: 1628-1630.

E164. The molecule of any one of E152-E163, wherein the first linker polypeptide comprises the amino acid sequence of SEQ ID NO: 1628.

E165. The molecule of any one of E152-E163, wherein the first linker polypeptide comprises the amino acid sequence of SEQ ID NO: 1629.

E166. The molecule of any one of E152-E163, wherein the first linker polypeptide comprises the amino acid sequence of SEQ ID NO: 1630.

E167. The molecule of any one of E152-E166, wherein the cysteine residue of the antibody that is conjugated to the N-terminus of the first linker polypeptide is at a position selected from the group consisting of 88 of a light chain, 384 of a heavy chain, and 487 of a heavy chain, according to AHo numbering.

E168. The molecule of E167, wherein the cysteine residue of the antibody that is conjugated to the N-terminus of the first linker polypeptide is at position 384 of a heavy chain, according to AHo numbering.

E169. The molecule of any one of E151-E168, further comprising a second polypeptide that agonizes a GCGR.

E170. The molecule of E169, wherein:
an ε-amino group of a lysine residue of the second polypeptide is covalently linked to a C-terminus of a second linker polypeptide; and
an N-terminus of the second linker polypeptide is conjugated to a cysteine residue of the antibody.

E171. The molecule of E169 or E170, wherein the second polypeptide is selected from the polypeptides of any one of E1-108.

E172. The molecule of any one of E169-E171, wherein the second polypeptide comprises the amino acid sequence of any one of SEQ ID NOs: 1587-1627.

E173. The molecule of any one of E169-E172, wherein the second polypeptide comprises the amino acid sequence of any one of SEQ ID NOs: 1587, 1592, 1596, 1615, or 1626.

E174. The molecule of any one of E169-E173, wherein the second polypeptide comprises the amino acid sequence of SEQ ID NO: 1587.

E175. The molecule of any one of E169-E173, wherein the second polypeptide comprises the amino acid sequence of SEQ ID NO: 1592.

E176. The molecule of any one of E169-E173, wherein the second polypeptide comprises the amino acid sequence of SEQ ID NO: 1596.

E177. The molecule of any one of E169-E173, wherein the second polypeptide comprises the amino acid sequence of SEQ ID NO: 1615.

E178. The molecule of any one of E169-E173, wherein the second polypeptide comprises the amino acid sequence of SEQ ID NO: 1626.

E179. The molecule of any one of E169-E178, wherein the first polypeptide has the same amino acid sequence as the second polypeptide.

E180. The molecule of any one of E170-E179, wherein the second linker polypeptide is a linear polypeptide.

E181. The molecule of any one of E170-E180, wherein the second linker polypeptide comprises the amino acid sequence of any one of SEQ ID NOs: 1628-1683.

E182. The molecule of any one of E170-E181, wherein the second linker polypeptide comprises the amino acid sequence of any one of SEQ ID NOs: 1628-1630.

E183. The molecule of any one of E170-E182, wherein the second linker polypeptide comprises the amino acid sequence of SEQ ID NO: 1628.

E184. The molecule of any one of E170-E182, wherein the second linker polypeptide comprises the amino acid sequence of SEQ ID NO: 1629.

E185. The molecule of any one of E170-E182, wherein the second linker polypeptide comprises the amino acid sequence of SEQ ID NO: 1630.

E186. The molecule of any one of E170-E185, wherein the first linker polypeptide has the same amino acid sequence as the second linker polypeptide.

E187. The molecule of any one of E170-E186, wherein the cysteine residue of the antibody that is conjugated to the N-terminus of the second linker polypeptide is at a position selected from the group consisting of 88 of a light chain, 384 of a heavy chain, and 487 of a heavy chain, according to AHo numbering.

E188. The molecule of E187, wherein the cysteine residue of the antibody that is conjugated to the N-terminus of the second linker polypeptide is at position 384 of a heavy chain, according to AHo numbering.

E189. The molecule of any one of E170-E187, wherein the cysteine residues of the antibody that are conjugated to the N-termini of the first linker polypeptide and the second linker polypeptide are at positions selected from the group consisting of 88 of both light chains, 384 of both heavy chains, and 487 of both heavy chains, according to AHo numbering.

E190. The molecule of E189, wherein the cysteine residues of the antibody that are conjugated to the N-termini of the first linker polypeptide and the second linker polypeptide are at positions 384 of both heavy chains, according to AHo numbering.

E191. The molecule of any one of E151-E190, wherein the antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise amino acid sequences selected from:
i. SEQ ID NO: 629, SEQ ID NO: 786, SEQ ID NO: 943, SEQ ID NO: 1100, SEQ ID NO: 1257, and SEQ ID NO: 1414, respectively;
ii. SEQ ID NO: 630, SEQ ID NO: 787, SEQ ID NO: 944, SEQ ID NO: 1101, SEQ ID NO: 1258, and SEQ ID NO: 1415, respectively;
iii. SEQ ID NO: 631, SEQ ID NO: 788, SEQ ID NO: 945, SEQ ID NO: 1102, SEQ ID NO: 1259, and SEQ ID NO: 1416, respectively;
iv. SEQ ID NO: 632, SEQ ID NO: 789, SEQ ID NO: 946, SEQ ID NO: 1103, SEQ ID NO: 1260, and SEQ ID NO: 1417, respectively;
v. SEQ ID NO: 633, SEQ ID NO: 790, SEQ ID NO: 947, SEQ ID NO: 1104, SEQ ID NO: 1261, and SEQ ID NO: 1418, respectively;
vi. SEQ ID NO: 634, SEQ ID NO: 791, SEQ ID NO: 948, SEQ ID NO: 1105, SEQ ID NO: 1262, and SEQ ID NO: 1419, respectively;
vii. SEQ ID NO: 635, SEQ ID NO: 792, SEQ ID NO: 949, SEQ ID NO: 1106, SEQ ID NO: 1263, and SEQ ID NO: 1420, respectively;
viii. SEQ ID NO: 636, SEQ ID NO: 793, SEQ ID NO: 950, SEQ ID NO: 1107, SEQ ID NO: 1264, and SEQ ID NO: 1421, respectively;
ix. SEQ ID NO: 637, SEQ ID NO: 794, SEQ ID NO: 951, SEQ ID NO: 1108, SEQ ID NO: 1265, and SEQ ID NO: 1422, respectively;
x. SEQ ID NO: 638, SEQ ID NO: 795, SEQ ID NO: 952, SEQ ID NO: 1109, SEQ ID NO: 1266, and SEQ ID NO: 1423, respectively;
xi. SEQ ID NO: 639, SEQ ID NO: 796, SEQ ID NO: 953, SEQ ID NO: 1110, SEQ ID NO: 1267, and SEQ ID NO: 1424, respectively;
xii. SEQ ID NO: 640, SEQ ID NO: 797, SEQ ID NO: 954, SEQ ID NO: 1111, SEQ ID NO: 1268, and SEQ ID NO: 1425, respectively;
xiii. SEQ ID NO: 641, SEQ ID NO: 798, SEQ ID NO: 955, SEQ ID NO: 1112, SEQ ID NO: 1269, and SEQ ID NO: 1426, respectively;
xiv. SEQ ID NO: 642, SEQ ID NO: 799, SEQ ID NO: 956, SEQ ID NO: 1113, SEQ ID NO: 1270, and SEQ ID NO: 1427, respectively;
xv. SEQ ID NO: 643, SEQ ID NO: 800, SEQ ID NO: 957, SEQ ID NO: 1114, SEQ ID NO: 1271, and SEQ ID NO: 1428, respectively;
xvi. SEQ ID NO: 644, SEQ ID NO: 801, SEQ ID NO: 958, SEQ ID NO: 1115, SEQ ID NO: 1272, and SEQ ID NO: 1429, respectively;
xvii. SEQ ID NO: 645, SEQ ID NO: 802, SEQ ID NO: 959, SEQ ID NO: 1116, SEQ ID NO: 1273, and SEQ ID NO: 1430, respectively;
xviii. SEQ ID NO: 646, SEQ ID NO: 803, SEQ ID NO: 960, SEQ ID NO: 1117, SEQ ID NO: 1274, and SEQ ID NO: 1431, respectively;
xix. SEQ ID NO: 647, SEQ ID NO: 804, SEQ ID NO: 961, SEQ ID NO: 1118, SEQ ID NO: 1275, and SEQ ID NO: 1432, respectively;
xx. SEQ ID NO: 648, SEQ ID NO: 805, SEQ ID NO: 962, SEQ ID NO: 1119, SEQ ID NO: 1276, and SEQ ID NO: 1433, respectively;
xxi. SEQ ID NO: 649, SEQ ID NO: 806, SEQ ID NO: 963, SEQ ID NO: 1120, SEQ ID NO: 1277, and SEQ ID NO: 1434, respectively;
xxii. SEQ ID NO: 650, SEQ ID NO: 807, SEQ ID NO: 964, SEQ ID NO: 1121, SEQ ID NO: 1278, and SEQ ID NO: 1435, respectively;
xxiii. SEQ ID NO: 651, SEQ ID NO: 808, SEQ ID NO: 965, SEQ ID NO: 1122, SEQ ID NO: 1279, and SEQ ID NO: 1436, respectively;
xxiv. SEQ ID NO: 652, SEQ ID NO: 809, SEQ ID NO: 966, SEQ ID NO: 1123, SEQ ID NO: 1280, and SEQ ID NO: 1437, respectively;
xxv. SEQ ID NO: 653, SEQ ID NO: 810, SEQ ID NO: 967, SEQ ID NO: 1124, SEQ ID NO: 1281, and SEQ ID NO: 1438, respectively;
xxvi. SEQ ID NO: 654, SEQ ID NO: 811, SEQ ID NO: 968, SEQ ID NO: 1125, SEQ ID NO: 1282, and SEQ ID NO: 1439, respectively;
xxvii. SEQ ID NO: 655, SEQ ID NO: 812, SEQ ID NO: 969, SEQ ID NO: 1126, SEQ ID NO: 1283, and SEQ ID NO: 1440, respectively;
xxviii. SEQ ID NO: 656, SEQ ID NO: 813, SEQ ID NO: 970, SEQ ID NO: 1127, SEQ ID NO: 1284, and SEQ ID NO: 1441, respectively;
xxix. SEQ ID NO: 657, SEQ ID NO: 814, SEQ ID NO: 971, SEQ ID NO: 1128, SEQ ID NO: 1285, and SEQ ID NO: 1442, respectively;
xxx. SEQ ID NO: 658, SEQ ID NO: 815, SEQ ID NO: 972, SEQ ID NO: 1129, SEQ ID NO: 1286, and SEQ ID NO: 1443, respectively;
xxxi. SEQ ID NO: 659, SEQ ID NO: 816, SEQ ID NO: 973, SEQ ID NO: 1130, SEQ ID NO: 1287, and SEQ ID NO: 1444, respectively;
xxxii. SEQ ID NO: 660, SEQ ID NO: 817, SEQ ID NO: 974, SEQ ID NO: 1131, SEQ ID NO: 1288, and SEQ ID NO: 1445, respectively;
xxxiii. SEQ ID NO: 661, SEQ ID NO: 818, SEQ ID NO: 975, SEQ ID NO: 1132, SEQ ID NO: 1289, and SEQ ID NO: 1446, respectively;
xxxiv. SEQ ID NO: 662, SEQ ID NO: 819, SEQ ID NO: 976, SEQ ID NO: 1133, SEQ ID NO: 1290, and SEQ ID NO: 1447, respectively;
xxxv. SEQ ID NO: 663, SEQ ID NO: 820, SEQ ID NO: 977, SEQ ID NO: 1134, SEQ ID NO: 1291, and SEQ ID NO: 1448, respectively;
xxxvi. SEQ ID NO: 664, SEQ ID NO: 821, SEQ ID NO: 978, SEQ ID NO: 1135, SEQ ID NO: 1292, and SEQ ID NO: 1449, respectively;
xxxvii. SEQ ID NO: 665, SEQ ID NO: 822, SEQ ID NO: 979, SEQ ID NO: 1136, SEQ ID NO: 1293, and SEQ ID NO: 1450, respectively;
xxxviii. SEQ ID NO: 666, SEQ ID NO: 823, SEQ ID NO: 980, SEQ ID NO: 1137, SEQ ID NO: 1294, and SEQ ID NO: 1451, respectively;
xxxix. SEQ ID NO: 667, SEQ ID NO: 824, SEQ ID NO: 981, SEQ ID NO: 1138, SEQ ID NO: 1295, and SEQ ID NO: 1452, respectively;
xl. SEQ ID NO: 668, SEQ ID NO: 825, SEQ ID NO: 982, SEQ ID NO: 1139, SEQ ID NO: 1296, and SEQ ID NO: 1453, respectively;
xli. SEQ ID NO: 669, SEQ ID NO: 826, SEQ ID NO: 983, SEQ ID NO: 1140, SEQ ID NO: 1297, and SEQ ID NO: 1454, respectively;
xlii. SEQ ID NO: 670, SEQ ID NO: 827, SEQ ID NO: 984, SEQ ID NO: 1141, SEQ ID NO: 1298, and SEQ ID NO: 1455, respectively;
xliii. SEQ ID NO: 671, SEQ ID NO: 828, SEQ ID NO: 985, SEQ ID NO: 1142, SEQ ID NO: 1299, and SEQ ID NO: 1456, respectively;
xliv. SEQ ID NO: 672, SEQ ID NO: 829, SEQ ID NO: 986, SEQ ID NO: 1143, SEQ ID NO: 1300, and SEQ ID NO: 1457, respectively;
xlv. SEQ ID NO: 673, SEQ ID NO: 830, SEQ ID NO: 987, SEQ ID NO: 1144, SEQ ID NO: 1301, and SEQ ID NO: 1458, respectively;
xlvi. SEQ ID NO: 674, SEQ ID NO: 831, SEQ ID NO: 988, SEQ ID NO: 1145, SEQ ID NO: 1302, and SEQ ID NO: 1459, respectively;
xlvii. SEQ ID NO: 675, SEQ ID NO: 832, SEQ ID NO: 989, SEQ ID NO: 1146, SEQ ID NO: 1303, and SEQ ID NO: 1460, respectively;
xlviii. SEQ ID NO: 676, SEQ ID NO: 833, SEQ ID NO: 990, SEQ ID NO: 1147, SEQ ID NO: 1304, and SEQ ID NO: 1461, respectively;
xlix. SEQ ID NO: 677, SEQ ID NO: 834, SEQ ID NO: 991, SEQ ID NO: 1148, SEQ ID NO: 1305, and SEQ ID NO: 1462, respectively;
l. SEQ ID NO: 678, SEQ ID NO: 835, SEQ ID NO: 992, SEQ ID NO: 1149, SEQ ID NO: 1306, and SEQ ID NO: 1463, respectively;
li. SEQ ID NO: 679, SEQ ID NO: 836, SEQ ID NO: 993, SEQ ID NO: 1150, SEQ ID NO: 1307, and SEQ ID NO: 1464, respectively;
lii. SEQ ID NO: 680, SEQ ID NO: 837, SEQ ID NO: 994, SEQ ID NO: 1151, SEQ ID NO: 1308, and SEQ ID NO: 1465, respectively;
liii. SEQ ID NO: 681, SEQ ID NO: 838, SEQ ID NO: 995, SEQ ID NO: 1152, SEQ ID NO: 1309, and SEQ ID NO: 1466, respectively;
liv. SEQ ID NO: 682, SEQ ID NO: 839, SEQ ID NO: 996, SEQ ID NO: 1153, SEQ ID NO: 1310, and SEQ ID NO: 1467, respectively;
lv. SEQ ID NO: 683, SEQ ID NO: 840, SEQ ID NO: 997, SEQ ID NO: 1154, SEQ ID NO: 1311, and SEQ ID NO: 1468, respectively;
lvi. SEQ ID NO: 684, SEQ ID NO: 841, SEQ ID NO: 998, SEQ ID NO: 1155, SEQ ID NO: 1312, and SEQ ID NO: 1469, respectively;
lvii. SEQ ID NO: 685, SEQ ID NO: 842, SEQ ID NO: 999, SEQ ID NO: 1156, SEQ ID NO: 1313, and SEQ ID NO: 1470, respectively;
lviii. SEQ ID NO: 686, SEQ ID NO: 843, SEQ ID NO: 1000, SEQ ID NO: 1157, SEQ ID NO: 1314, and SEQ ID NO: 1471, respectively;
lix. SEQ ID NO: 687, SEQ ID NO: 844, SEQ ID NO: 1001, SEQ ID NO: 1158, SEQ ID NO: 1315, and SEQ ID NO: 1472, respectively;
lx. SEQ ID NO: 688, SEQ ID NO: 845, SEQ ID NO: 1002, SEQ ID NO: 1159, SEQ ID NO: 1316, and SEQ ID NO: 1473, respectively;
lxi. SEQ ID NO: 689, SEQ ID NO: 846, SEQ ID NO: 1003, SEQ ID NO: 1160, SEQ ID NO: 1317, and SEQ ID NO: 1474, respectively;
lxii. SEQ ID NO: 690, SEQ ID NO: 847, SEQ ID NO: 1004, SEQ ID NO: 1161, SEQ ID NO: 1318, and SEQ ID NO: 1475, respectively;
lxiii. SEQ ID NO: 691, SEQ ID NO: 848, SEQ ID NO: 1005, SEQ ID NO: 1162, SEQ ID NO: 1319, and SEQ ID NO: 1476, respectively;
lxiv. SEQ ID NO: 692, SEQ ID NO: 849, SEQ ID NO: 1006, SEQ ID NO: 1163, SEQ ID NO: 1320, and SEQ ID NO: 1477, respectively;
lxv. SEQ ID NO: 693, SEQ ID NO: 850, SEQ ID NO: 1007, SEQ ID NO: 1164, SEQ ID NO: 1321, and SEQ ID NO: 1478, respectively;
lxvi. SEQ ID NO: 694, SEQ ID NO: 851, SEQ ID NO: 1008, SEQ ID NO: 1165, SEQ ID NO: 1322, and SEQ ID NO: 1479, respectively;
lxvii. SEQ ID NO: 695, SEQ ID NO: 852, SEQ ID NO: 1009, SEQ ID NO: 1166, SEQ ID NO: 1323, and SEQ ID NO: 1480, respectively;
lxviii. SEQ ID NO: 696, SEQ ID NO: 853, SEQ ID NO: 1010, SEQ ID NO: 1167, SEQ ID NO: 1324, and SEQ ID NO: 1481, respectively;
lxix. SEQ ID NO: 697, SEQ ID NO: 854, SEQ ID NO: 1011, SEQ ID NO: 1168, SEQ ID NO: 1325, and SEQ ID NO: 1482, respectively;
lxx. SEQ ID NO: 698, SEQ ID NO: 855, SEQ ID NO: 1012, SEQ ID NO: 1169, SEQ ID NO: 1326, and SEQ ID NO: 1483, respectively;
lxxi. SEQ ID NO: 699, SEQ ID NO: 856, SEQ ID NO: 1013, SEQ ID NO: 1170, SEQ ID NO: 1327, and SEQ ID NO: 1484, respectively;
lxxii. SEQ ID NO: 700, SEQ ID NO: 857, SEQ ID NO: 1014, SEQ ID NO: 1171, SEQ ID NO: 1328, and SEQ ID NO: 1485, respectively;
lxxiii. SEQ ID NO: 701, SEQ ID NO: 858, SEQ ID NO: 1015, SEQ ID NO: 1172, SEQ ID NO: 1329, and SEQ ID NO: 1486, respectively;
lxxiv. SEQ ID NO: 702, SEQ ID NO: 859, SEQ ID NO: 1016, SEQ ID NO: 1173, SEQ ID NO: 1330, and SEQ ID NO: 1487, respectively;
lxxv. SEQ ID NO: 703, SEQ ID NO: 860, SEQ ID NO: 1017, SEQ ID NO: 1174, SEQ ID NO: 1331, and SEQ ID NO: 1488, respectively;
lxxvi. SEQ ID NO: 704, SEQ ID NO: 861, SEQ ID NO: 1018, SEQ ID NO: 1175, SEQ ID NO: 1332, and SEQ ID NO: 1489, respectively;
lxxvii. SEQ ID NO: 705, SEQ ID NO: 862, SEQ ID NO: 1019, SEQ ID NO: 1176, SEQ ID NO: 1333, and SEQ ID NO: 1490, respectively;
lxxviii. SEQ ID NO: 706, SEQ ID NO: 863, SEQ ID NO: 1020, SEQ ID NO: 1177, SEQ ID NO: 1334, and SEQ ID NO: 1491, respectively;
lxxix. SEQ ID NO: 707, SEQ ID NO: 864, SEQ ID NO: 1021, SEQ ID NO: 1178, SEQ ID NO: 1335, and SEQ ID NO: 1492, respectively;
lxxx. SEQ ID NO: 708, SEQ ID NO: 865, SEQ ID NO: 1022, SEQ ID NO: 1179, SEQ ID NO: 1336, and SEQ ID NO: 1493, respectively;
lxxxi. SEQ ID NO: 709, SEQ ID NO: 866, SEQ ID NO: 1023, SEQ ID NO: 1180, SEQ ID NO: 1337, and SEQ ID NO: 1494, respectively;
lxxxii. SEQ ID NO: 710, SEQ ID NO: 867, SEQ ID NO: 1024, SEQ ID NO: 1181, SEQ ID NO: 1338, and SEQ ID NO: 1495, respectively;
lxxxiii. SEQ ID NO: 711, SEQ ID NO: 868, SEQ ID NO: 1025, SEQ ID NO: 1182, SEQ ID NO: 1339, and SEQ ID NO: 1496, respectively;
lxxxiv. SEQ ID NO: 712, SEQ ID NO: 869, SEQ ID NO: 1026, SEQ ID NO: 1183, SEQ ID NO: 1340, and SEQ ID NO: 1497, respectively;
lxxxv. SEQ ID NO: 713, SEQ ID NO: 870, SEQ ID NO: 1027, SEQ ID NO: 1184, SEQ ID NO: 1341, and SEQ ID NO: 1498, respectively;
lxxxvi. SEQ ID NO: 714, SEQ ID NO: 871, SEQ ID NO: 1028, SEQ ID NO: 1185, SEQ ID NO: 1342, and SEQ ID NO: 1499, respectively;
lxxxvii. SEQ ID NO: 715, SEQ ID NO: 872, SEQ ID NO: 1029, SEQ ID NO: 1186, SEQ ID NO: 1343, and SEQ ID NO: 1500, respectively;
lxxxviii. SEQ ID NO: 716, SEQ ID NO: 873, SEQ ID NO: 1030, SEQ ID NO: 1187, SEQ ID NO: 1344, and SEQ ID NO: 1501, respectively;
lxxxix. SEQ ID NO: 717, SEQ ID NO: 874, SEQ ID NO: 1031, SEQ ID NO: 1188, SEQ ID NO: 1345, and SEQ ID NO: 1502, respectively;
xc. SEQ ID NO: 718, SEQ ID NO: 875, SEQ ID NO: 1032, SEQ ID NO: 1189, SEQ ID NO: 1346, and SEQ ID NO: 1503, respectively;
xci. SEQ ID NO: 719, SEQ ID NO: 876, SEQ ID NO: 1033, SEQ ID NO: 1190, SEQ ID NO: 1347, and SEQ ID NO: 1504, respectively;
xcii. SEQ ID NO: 720, SEQ ID NO: 877, SEQ ID NO: 1034, SEQ ID NO: 1191, SEQ ID NO: 1348, and SEQ ID NO: 1505, respectively;
xciii. SEQ ID NO: 721, SEQ ID NO: 878, SEQ ID NO: 1035, SEQ ID NO: 1192, SEQ ID NO: 1349, and SEQ ID NO: 1506, respectively;
xciv. SEQ ID NO: 722, SEQ ID NO: 879, SEQ ID NO: 1036, SEQ ID NO: 1193, SEQ ID NO: 1350, and SEQ ID NO: 1507, respectively;
xcv. SEQ ID NO: 723, SEQ ID NO: 880, SEQ ID NO: 1037, SEQ ID NO: 1194, SEQ ID NO: 1351, and SEQ ID NO: 1508, respectively;
xcvi. SEQ ID NO: 724, SEQ ID NO: 881, SEQ ID NO: 1038, SEQ ID NO: 1195, SEQ ID NO: 1352, and SEQ ID NO: 1509, respectively;
xcvii. SEQ ID NO: 725, SEQ ID NO: 882, SEQ ID NO: 1039, SEQ ID NO: 1196, SEQ ID NO: 1353, and SEQ ID NO: 1510, respectively;
xcviii. SEQ ID NO: 726, SEQ ID NO: 883, SEQ ID NO: 1040, SEQ ID NO: 1197, SEQ ID NO: 1354, and SEQ ID NO: 1511, respectively;
xcix. SEQ ID NO: 727, SEQ ID NO: 884, SEQ ID NO: 1041, SEQ ID NO: 1198, SEQ ID NO: 1355, and SEQ ID NO: 1512, respectively;
c. SEQ ID NO: 728, SEQ ID NO: 885, SEQ ID NO: 1042, SEQ ID NO: 1199, SEQ ID NO: 1356, and SEQ ID NO: 1513, respectively;
ci. SEQ ID NO: 729, SEQ ID NO: 886, SEQ ID NO: 1043, SEQ ID NO: 1200, SEQ ID NO: 1357, and SEQ ID NO: 1514, respectively;
cii. SEQ ID NO: 730, SEQ ID NO: 887, SEQ ID NO: 1044, SEQ ID NO: 1201, SEQ ID NO: 1358, and SEQ ID NO: 1515, respectively;
ciii. SEQ ID NO: 731, SEQ ID NO: 888, SEQ ID NO: 1045, SEQ ID NO: 1202, SEQ ID NO: 1359, and SEQ ID NO: 1516, respectively;
civ. SEQ ID NO: 732, SEQ ID NO: 889, SEQ ID NO: 1046, SEQ ID NO: 1203, SEQ ID NO: 1360, and SEQ ID NO: 1517, respectively;
cv. SEQ ID NO: 733, SEQ ID NO: 890, SEQ ID NO: 1047, SEQ ID NO: 1204, SEQ ID NO: 1361, and SEQ ID NO: 1518, respectively;
cvi. SEQ ID NO: 734, SEQ ID NO: 891, SEQ ID NO: 1048, SEQ ID NO: 1205, SEQ ID NO: 1362, and SEQ ID NO: 1519, respectively;
cvii. SEQ ID NO: 735, SEQ ID NO: 892, SEQ ID NO: 1049, SEQ ID NO: 1206, SEQ ID NO: 1363, and SEQ ID NO: 1520, respectively;
cviii. SEQ ID NO: 736, SEQ ID NO: 893, SEQ ID NO: 1050, SEQ ID NO: 1207, SEQ ID NO: 1364, and SEQ ID NO: 1521, respectively;
cix. SEQ ID NO: 737, SEQ ID NO: 894, SEQ ID NO: 1051, SEQ ID NO: 1208, SEQ ID NO: 1365, and SEQ ID NO: 1522, respectively;
cx. SEQ ID NO: 738, SEQ ID NO: 895, SEQ ID NO: 1052, SEQ ID NO: 1209, SEQ ID NO: 1366, and SEQ ID NO: 1523, respectively;
cxi. SEQ ID NO: 739, SEQ ID NO: 896, SEQ ID NO: 1053, SEQ ID NO: 1210, SEQ ID NO: 1367, and SEQ ID NO: 1524, respectively;
cxii. SEQ ID NO: 740, SEQ ID NO: 897, SEQ ID NO: 1054, SEQ ID NO: 1211, SEQ ID NO: 1368, and SEQ ID NO: 1525, respectively;
cxiii. SEQ ID NO: 741, SEQ ID NO: 898, SEQ ID NO: 1055, SEQ ID NO: 1212, SEQ ID NO: 1369, and SEQ ID NO: 1526, respectively;
cxiv. SEQ ID NO: 742, SEQ ID NO: 899, SEQ ID NO: 1056, SEQ ID NO: 1213, SEQ ID NO: 1370, and SEQ ID NO: 1527, respectively;
cxv. SEQ ID NO: 743, SEQ ID NO: 900, SEQ ID NO: 1057, SEQ ID NO: 1214, SEQ ID NO: 1371, and SEQ ID NO: 1528, respectively;
cxvi. SEQ ID NO: 744, SEQ ID NO: 901, SEQ ID NO: 1058, SEQ ID NO: 1215, SEQ ID NO: 1372, and SEQ ID NO: 1529, respectively;
cxvii. SEQ ID NO: 745, SEQ ID NO: 902, SEQ ID NO: 1059, SEQ ID NO: 1216, SEQ ID NO: 1373, and SEQ ID NO: 1530, respectively;
cxviii. SEQ ID NO: 746, SEQ ID NO: 903, SEQ ID NO: 1060, SEQ ID NO: 1217, SEQ ID NO: 1374, and SEQ ID NO: 1531, respectively;
cxix. SEQ ID NO: 747, SEQ ID NO: 904, SEQ ID NO: 1061, SEQ ID NO: 1218, SEQ ID NO: 1375, and SEQ ID NO: 1532, respectively;
cxx. SEQ ID NO: 748, SEQ ID NO: 905, SEQ ID NO: 1062, SEQ ID NO: 1219, SEQ ID NO: 1376, and SEQ ID NO: 1533, respectively;
cxxi. SEQ ID NO: 749, SEQ ID NO: 906, SEQ ID NO: 1063, SEQ ID NO: 1220, SEQ ID NO: 1377, and SEQ ID NO: 1534, respectively;
cxxii. SEQ ID NO: 750, SEQ ID NO: 907, SEQ ID NO: 1064, SEQ ID NO: 1221, SEQ ID NO: 1378, and SEQ ID NO: 1535, respectively;
cxxiii. SEQ ID NO: 751, SEQ ID NO: 908, SEQ ID NO: 1065, SEQ ID NO: 1222, SEQ ID NO: 1379, and SEQ ID NO: 1536, respectively;
cxxiv. SEQ ID NO: 752, SEQ ID NO: 909, SEQ ID NO: 1066, SEQ ID NO: 1223, SEQ ID NO: 1380, and SEQ ID NO: 1537, respectively;
cxxv. SEQ ID NO: 753, SEQ ID NO: 910, SEQ ID NO: 1067, SEQ ID NO: 1224, SEQ ID NO: 1381, and SEQ ID NO: 1538, respectively;
cxxvi. SEQ ID NO: 754, SEQ ID NO: 911, SEQ ID NO: 1068, SEQ ID NO: 1225, SEQ ID NO: 1382, and SEQ ID NO: 1539, respectively;
cxxvii. SEQ ID NO: 755, SEQ ID NO: 912, SEQ ID NO: 1069, SEQ ID NO: 1226, SEQ ID NO: 1383, and SEQ ID NO: 1540, respectively;
cxxviii. SEQ ID NO: 756, SEQ ID NO: 913, SEQ ID NO: 1070, SEQ ID NO: 1227, SEQ ID NO: 1384, and SEQ ID NO: 1541, respectively;
cxxix. SEQ ID NO: 757, SEQ ID NO: 914, SEQ ID NO: 1071, SEQ ID NO: 1228, SEQ ID NO: 1385, and SEQ ID NO: 1542, respectively;
cxxx. SEQ ID NO: 758, SEQ ID NO: 915, SEQ ID NO: 1072, SEQ ID NO: 1229, SEQ ID NO: 1386, and SEQ ID NO: 1543, respectively;
cxxxi. SEQ ID NO: 759, SEQ ID NO: 916, SEQ ID NO: 1073, SEQ ID NO: 1230, SEQ ID NO: 1387, and SEQ ID NO: 1544, respectively;
cxxxii. SEQ ID NO: 760, SEQ ID NO: 917, SEQ ID NO: 1074, SEQ ID NO: 1231, SEQ ID NO: 1388, and SEQ ID NO: 1545, respectively;
cxxxiii. SEQ ID NO: 761, SEQ ID NO: 918, SEQ ID NO: 1075, SEQ ID NO: 1232, SEQ ID NO: 1389, and SEQ ID NO: 1546, respectively;
cxxxiv. SEQ ID NO: 762, SEQ ID NO: 919, SEQ ID NO: 1076, SEQ ID NO: 1233, SEQ ID NO: 1390, and SEQ ID NO: 1547, respectively;
cxxxv. SEQ ID NO: 763, SEQ ID NO: 920, SEQ ID NO: 1077, SEQ ID NO: 1234, SEQ ID NO: 1391, and SEQ ID NO: 1548, respectively;
cxxxvi. SEQ ID NO: 764, SEQ ID NO: 921, SEQ ID NO: 1078, SEQ ID NO: 1235, SEQ ID NO: 1392, and SEQ ID NO: 1549, respectively;
cxxxvii. SEQ ID NO: 765, SEQ ID NO: 922, SEQ ID NO: 1079, SEQ ID NO: 1236, SEQ ID NO: 1393, and SEQ ID NO: 1550, respectively;
cxxxviii. SEQ ID NO: 766, SEQ ID NO: 923, SEQ ID NO: 1080, SEQ ID NO: 1237, SEQ ID NO: 1394, and SEQ ID NO: 1551, respectively;
cxxxix. SEQ ID NO: 767, SEQ ID NO: 924, SEQ ID NO: 1081, SEQ ID NO: 1238, SEQ ID NO: 1395, and SEQ ID NO: 1552, respectively;
cxl. SEQ ID NO: 768, SEQ ID NO: 925, SEQ ID NO: 1082, SEQ ID NO: 1239, SEQ ID NO: 1396, and SEQ ID NO: 1553, respectively;
cxli. SEQ ID NO: 769, SEQ ID NO: 926, SEQ ID NO: 1083, SEQ ID NO: 1240, SEQ ID NO: 1397, and SEQ ID NO: 1554, respectively;
cxlii. SEQ ID NO: 770, SEQ ID NO: 927, SEQ ID NO: 1084, SEQ ID NO: 1241, SEQ ID NO: 1398, and SEQ ID NO: 1555, respectively;
cxliii. SEQ ID NO: 771, SEQ ID NO: 928, SEQ ID NO: 1085, SEQ ID NO: 1242, SEQ ID NO: 1399, and SEQ ID NO: 1556, respectively;
cxliv. SEQ ID NO: 772, SEQ ID NO: 929, SEQ ID NO: 1086, SEQ ID NO: 1243, SEQ ID NO: 1400, and SEQ ID NO: 1557, respectively;
cxlv. SEQ ID NO: 773, SEQ ID NO: 930, SEQ ID NO: 1087, SEQ ID NO: 1244, SEQ ID NO: 1401, and SEQ ID NO: 1558, respectively;
cxlvi. SEQ ID NO: 774, SEQ ID NO: 931, SEQ ID NO: 1088, SEQ ID NO: 1245, SEQ ID NO: 1402, and SEQ ID NO: 1559, respectively;
cxlvii. SEQ ID NO: 775, SEQ ID NO: 932, SEQ ID NO: 1089, SEQ ID NO: 1246, SEQ ID NO: 1403, and SEQ ID NO: 1560, respectively;
cxlviii. SEQ ID NO: 776, SEQ ID NO: 933, SEQ ID NO: 1090, SEQ ID NO: 1247, SEQ ID NO: 1404, and SEQ ID NO: 1561, respectively;
cxlix. SEQ ID NO: 777, SEQ ID NO: 934, SEQ ID NO: 1091, SEQ ID NO: 1248, SEQ ID NO: 1405, and SEQ ID NO: 1562, respectively;
cl. SEQ ID NO: 778, SEQ ID NO: 935, SEQ ID NO: 1092, SEQ ID NO: 1249, SEQ ID NO: 1406, and SEQ ID NO: 1563, respectively;
cli. SEQ ID NO: 779, SEQ ID NO: 936, SEQ ID NO: 1093, SEQ ID NO: 1250, SEQ ID NO: 1407, and SEQ ID NO: 1564, respectively;
clii. SEQ ID NO: 780, SEQ ID NO: 937, SEQ ID NO: 1094, SEQ ID NO: 1251, SEQ ID NO: 1408, and SEQ ID NO: 1565, respectively;
cliii. SEQ ID NO: 781, SEQ ID NO: 938, SEQ ID NO: 1095, SEQ ID NO: 1252, SEQ ID NO: 1409, and SEQ ID NO: 1566, respectively;
cliv. SEQ ID NO: 782, SEQ ID NO: 939, SEQ ID NO: 1096, SEQ ID NO: 1253, SEQ ID NO: 1410, and SEQ ID NO: 1567, respectively;
clv. SEQ ID NO: 783, SEQ ID NO: 940, SEQ ID NO: 1097, SEQ ID NO: 1254, SEQ ID NO: 1411, and SEQ ID NO: 1568, respectively;
clvi. SEQ ID NO: 784, SEQ ID NO: 941, SEQ ID NO: 1098, SEQ ID NO: 1255, SEQ ID NO: 1412, and SEQ ID NO: 1569, respectively; and
clvii. SEQ ID NO: 785, SEQ ID NO: 942, SEQ ID NO: 1099, SEQ ID NO: 1256, SEQ ID NO: 1413, and SEQ ID NO: 1570, respectively,
preferably wherein the CDRL1, the CDRL2, the CDRL3, the CDRH1, the CDRH2, and the CDRH3 comprise the amino acid sequences of SEQ ID NO: 702, SEQ ID NO: 859, SEQ ID NO: 1016, SEQ ID NO: 1173, SEQ ID NO: 1330, and SEQ ID NO: 1487, respectively.

E192. The molecule of any one of E151-E191, wherein the antibody comprises a light chain variable region and a heavy chain variable region, wherein the light chain variable region and the heavy chain variable region comprise amino acid sequences selected from:
i. SEQ ID NO: 1 and SEQ ID NO: 158, respectively;
ii. SEQ ID NO: 2 and SEQ ID NO: 159, respectively;
iii. SEQ ID NO: 3 and SEQ ID NO: 160, respectively;
iv. SEQ ID NO: 4 and SEQ ID NO: 161, respectively;
v. SEQ ID NO: 5 and SEQ ID NO: 162, respectively;
vi. SEQ ID NO: 6 and SEQ ID NO: 163, respectively;
vii. SEQ ID NO: 7 and SEQ ID NO: 164, respectively;
viii. SEQ ID NO: 8 and SEQ ID NO: 165, respectively;
ix. SEQ ID NO: 9 and SEQ ID NO: 166, respectively;
x. SEQ ID NO: 10 and SEQ ID NO: 167, respectively;
xi. SEQ ID NO: 11 and SEQ ID NO: 168, respectively;
xii. SEQ ID NO: 12 and SEQ ID NO: 169, respectively;
xiii. SEQ ID NO: 13 and SEQ ID NO: 170, respectively;
xiv. SEQ ID NO: 14 and SEQ ID NO: 171, respectively;
xv. SEQ ID NO: 15 and SEQ ID NO: 172, respectively;
xvi. SEQ ID NO: 16 and SEQ ID NO: 173, respectively;
xvii. SEQ ID NO: 17 and SEQ ID NO: 174, respectively;
xviii. SEQ ID NO: 18 and SEQ ID NO: 175, respectively;
xix. SEQ ID NO: 19 and SEQ ID NO: 176, respectively;
xx. SEQ ID NO: 20 and SEQ ID NO: 177, respectively;
xxi. SEQ ID NO: 21 and SEQ ID NO: 178, respectively;
xxii. SEQ ID NO: 22 and SEQ ID NO: 179, respectively;
xxiii. SEQ ID NO: 23 and SEQ ID NO: 180, respectively;
xxiv. SEQ ID NO: 24 and SEQ ID NO: 181, respectively;
xxv. SEQ ID NO: 25 and SEQ ID NO: 182, respectively;
xxvi. SEQ ID NO: 26 and SEQ ID NO: 183, respectively;
xxvii. SEQ ID NO: 27 and SEQ ID NO: 184, respectively;
xxviii. SEQ ID NO: 28 and SEQ ID NO: 185, respectively;
xxix. SEQ ID NO: 29 and SEQ ID NO: 186, respectively;
xxx. SEQ ID NO: 30 and SEQ ID NO: 187, respectively;
xxxi. SEQ ID NO: 31 and SEQ ID NO: 188, respectively;
xxxii. SEQ ID NO: 32 and SEQ ID NO: 189, respectively;
xxxiii. SEQ ID NO: 33 and SEQ ID NO: 190, respectively;
xxxiv. SEQ ID NO: 34 and SEQ ID NO: 191, respectively;
xxxv. SEQ ID NO: 35 and SEQ ID NO: 192, respectively;
xxxvi. SEQ ID NO: 36 and SEQ ID NO: 193, respectively;
xxxvii. SEQ ID NO: 37 and SEQ ID NO: 194, respectively;
xxxviii. SEQ ID NO: 38 and SEQ ID NO: 195, respectively;
xxxix. SEQ ID NO: 39 and SEQ ID NO: 196, respectively;
xl. SEQ ID NO: 40 and SEQ ID NO: 197, respectively;
xli. SEQ ID NO: 41 and SEQ ID NO: 198, respectively;
xlii. SEQ ID NO: 42 and SEQ ID NO: 199, respectively;
xliii. SEQ ID NO: 43 and SEQ ID NO: 200, respectively;
xliv. SEQ ID NO: 44 and SEQ ID NO: 201, respectively;
xlv. SEQ ID NO: 45 and SEQ ID NO: 202, respectively;
xlvi. SEQ ID NO: 46 and SEQ ID NO: 203, respectively;
xlvii. SEQ ID NO: 47 and SEQ ID NO: 204, respectively;
xlviii. SEQ ID NO: 48 and SEQ ID NO: 205, respectively;
xlix. SEQ ID NO: 49 and SEQ ID NO: 206, respectively;
l. SEQ ID NO: 50 and SEQ ID NO: 207, respectively;
li. SEQ ID NO: 51 and SEQ ID NO: 208, respectively;
lii. SEQ ID NO: 52 and SEQ ID NO: 209, respectively;
liii. SEQ ID NO: 53 and SEQ ID NO: 210, respectively;
liv. SEQ ID NO: 54 and SEQ ID NO: 211, respectively;
lv. SEQ ID NO: 55 and SEQ ID NO: 212, respectively;
lvi. SEQ ID NO: 56 and SEQ ID NO: 213, respectively;
lvii. SEQ ID NO: 57 and SEQ ID NO: 214, respectively;
lviii. SEQ ID NO: 58 and SEQ ID NO: 215, respectively;
lix. SEQ ID NO: 59 and SEQ ID NO: 216, respectively;
lx. SEQ ID NO: 60 and SEQ ID NO: 217, respectively;
lxi. SEQ ID NO: 61 and SEQ ID NO: 218, respectively;
lxii. SEQ ID NO: 62 and SEQ ID NO: 219, respectively;
lxiii. SEQ ID NO: 63 and SEQ ID NO: 220, respectively;
lxiv. SEQ ID NO: 64 and SEQ ID NO: 221, respectively;
lxv. SEQ ID NO: 65 and SEQ ID NO: 222, respectively;
lxvi. SEQ ID NO: 66 and SEQ ID NO: 223, respectively;
lxvii. SEQ ID NO: 67 and SEQ ID NO: 224, respectively;
lxviii. SEQ ID NO: 68 and SEQ ID NO: 225, respectively;
lxix. SEQ ID NO: 69 and SEQ ID NO: 226, respectively;
lxx. SEQ ID NO: 70 and SEQ ID NO: 227, respectively;
lxxi. SEQ ID NO: 71 and SEQ ID NO: 228, respectively;
lxxii. SEQ ID NO: 72 and SEQ ID NO: 229, respectively;
lxxiii. SEQ ID NO: 73 and SEQ ID NO: 230, respectively;
lxxiv. SEQ ID NO: 74 and SEQ ID NO: 231, respectively;
lxxv. SEQ ID NO: 75 and SEQ ID NO: 232, respectively;
lxxvi. SEQ ID NO: 76 and SEQ ID NO: 233, respectively;
lxxvii. SEQ ID NO: 77 and SEQ ID NO: 234, respectively;
lxxviii. SEQ ID NO: 78 and SEQ ID NO: 235, respectively;
lxxix. SEQ ID NO: 79 and SEQ ID NO: 236, respectively;
lxxx. SEQ ID NO: 80 and SEQ ID NO: 237, respectively;
lxxxi. SEQ ID NO: 81 and SEQ ID NO: 238, respectively;
lxxxii. SEQ ID NO: 82 and SEQ ID NO: 239, respectively;
lxxxiii. SEQ ID NO: 83 and SEQ ID NO: 240, respectively;
lxxxiv. SEQ ID NO: 84 and SEQ ID NO: 241, respectively;
lxxxv. SEQ ID NO: 85 and SEQ ID NO: 242, respectively;
lxxxvi. SEQ ID NO: 86 and SEQ ID NO: 243, respectively;
lxxxvii. SEQ ID NO: 87 and SEQ ID NO: 244, respectively;
lxxxviii. SEQ ID NO: 88 and SEQ ID NO: 245, respectively;
lxxxix. SEQ ID NO: 89 and SEQ ID NO: 246, respectively;
xc. SEQ ID NO: 90 and SEQ ID NO: 247, respectively;
xci. SEQ ID NO: 91 and SEQ ID NO: 248, respectively;
xcii. SEQ ID NO: 92 and SEQ ID NO: 249, respectively;
xciii. SEQ ID NO: 93 and SEQ ID NO: 250, respectively;
xciv. SEQ ID NO: 94 and SEQ ID NO: 251, respectively;
xcv. SEQ ID NO: 95 and SEQ ID NO: 252, respectively;
xcvi. SEQ ID NO: 96 and SEQ ID NO: 253, respectively;
xcvii. SEQ ID NO: 97 and SEQ ID NO: 254, respectively;
xcviii. SEQ ID NO: 98 and SEQ ID NO: 255, respectively;
xcix. SEQ ID NO: 99 and SEQ ID NO: 256, respectively;
c. SEQ ID NO: 100 and SEQ ID NO: 257, respectively;
ci. SEQ ID NO: 101 and SEQ ID NO: 258, respectively;
cii. SEQ ID NO: 102 and SEQ ID NO: 259, respectively;
ciii. SEQ ID NO: 103 and SEQ ID NO: 260, respectively;
civ. SEQ ID NO: 104 and SEQ ID NO: 261, respectively;
cv. SEQ ID NO: 105 and SEQ ID NO: 262, respectively;
cvi. SEQ ID NO: 106 and SEQ ID NO: 263, respectively;
cvii. SEQ ID NO: 107 and SEQ ID NO: 264, respectively;
cviii. SEQ ID NO: 108 and SEQ ID NO: 265, respectively;
cix. SEQ ID NO: 109 and SEQ ID NO: 266, respectively;
cx. SEQ ID NO: 110 and SEQ ID NO: 267, respectively;
cxi. SEQ ID NO: 111 and SEQ ID NO: 268, respectively;
cxii. SEQ ID NO: 112 and SEQ ID NO: 269, respectively;
cxiii. SEQ ID NO: 113 and SEQ ID NO: 270, respectively;
cxiv. SEQ ID NO: 114 and SEQ ID NO: 271, respectively;
cxv. SEQ ID NO: 115 and SEQ ID NO: 272, respectively;
cxvi. SEQ ID NO: 116 and SEQ ID NO: 273, respectively;
cxvii. SEQ ID NO: 117 and SEQ ID NO: 274, respectively;
cxviii. SEQ ID NO: 118 and SEQ ID NO: 275, respectively;
cxix. SEQ ID NO: 119 and SEQ ID NO: 276, respectively;
cxx. SEQ ID NO: 120 and SEQ ID NO: 277, respectively;
cxxi. SEQ ID NO: 121 and SEQ ID NO: 278, respectively;
cxxii. SEQ ID NO: 122 and SEQ ID NO: 279, respectively;
cxxiii. SEQ ID NO: 123 and SEQ ID NO: 280, respectively;
cxxiv. SEQ ID NO: 124 and SEQ ID NO: 281, respectively;
cxxv. SEQ ID NO: 125 and SEQ ID NO: 282, respectively;
cxxvi. SEQ ID NO: 126 and SEQ ID NO: 283, respectively;
cxxvii. SEQ ID NO: 127 and SEQ ID NO: 284, respectively;
cxxviii. SEQ ID NO: 128 and SEQ ID NO: 285, respectively;
cxxix. SEQ ID NO: 129 and SEQ ID NO: 286, respectively;
cxxx. SEQ ID NO: 130 and SEQ ID NO: 287, respectively;
cxxxi. SEQ ID NO: 131 and SEQ ID NO: 288, respectively;
cxxxii. SEQ ID NO: 132 and SEQ ID NO: 289, respectively;
cxxxiii. SEQ ID NO: 133 and SEQ ID NO: 290, respectively;
cxxxiv. SEQ ID NO: 134 and SEQ ID NO: 291, respectively;
cxxxv. SEQ ID NO: 135 and SEQ ID NO: 292, respectively;
cxxxvi. SEQ ID NO: 136 and SEQ ID NO: 293, respectively;
cxxxvii. SEQ ID NO: 137 and SEQ ID NO: 294, respectively;
cxxxviii. SEQ ID NO: 138 and SEQ ID NO: 295, respectively;
cxxxix. SEQ ID NO: 139 and SEQ ID NO: 296, respectively;
cxl. SEQ ID NO: 140 and SEQ ID NO: 297, respectively;
cxli. SEQ ID NO: 141 and SEQ ID NO: 298, respectively;
cxlii. SEQ ID NO: 142 and SEQ ID NO: 299, respectively;
cxliii. SEQ ID NO: 143 and SEQ ID NO: 300, respectively;
cxliv. SEQ ID NO: 144 and SEQ ID NO: 301, respectively;
cxlv. SEQ ID NO: 145 and SEQ ID NO: 302, respectively;
cxlvi. SEQ ID NO: 146 and SEQ ID NO: 303, respectively;
cxlvii. SEQ ID NO: 147 and SEQ ID NO: 304, respectively;
cxlviii. SEQ ID NO: 148 and SEQ ID NO: 305, respectively;
cxlix. SEQ ID NO: 149 and SEQ ID NO: 306, respectively;
cl. SEQ ID NO: 150 and SEQ ID NO: 307, respectively;
cli. SEQ ID NO: 151 and SEQ ID NO: 308, respectively;
clii. SEQ ID NO: 152 and SEQ ID NO: 309, respectively;
cliii. SEQ ID NO: 153 and SEQ ID NO: 310, respectively;
cliv. SEQ ID NO: 154 and SEQ ID NO: 311, respectively;
clv. SEQ ID NO: 155 and SEQ ID NO: 312, respectively;
clvi. SEQ ID NO: 156 and SEQ ID NO: 313, respectively; and
clvii. SEQ ID NO: 157 and SEQ ID NO: 314, respectively,
preferably wherein the light chain variable region and the heavy chain variable region comprise the amino acid sequences of SEQ ID NO: 74 and SEQ ID NO: 231, respectively.

E193. The molecule of anyone of E151-167, E169-E187, E188, E189, E191, or E192, wherein the antibody comprises a light chain and a heavy chain, wherein the light chain and heavy chain comprise amino acid sequences selected from:
i. SEQ ID NO: 315 and SEQ ID NO: 472, respectively;
ii. SEQ ID NO: 316 and SEQ ID NO: 473, respectively;
iii. SEQ ID NO: 317 and SEQ ID NO: 474, respectively;
iv. SEQ ID NO: 318 and SEQ ID NO: 475, respectively;
v. SEQ ID NO: 319 and SEQ ID NO: 476, respectively;
vi. SEQ ID NO: 320 and SEQ ID NO: 477, respectively;
vii. SEQ ID NO: 321 and SEQ ID NO: 478, respectively;
viii. SEQ ID NO: 322 and SEQ ID NO: 479, respectively;
ix. SEQ ID NO: 323 and SEQ ID NO: 480, respectively;
x. SEQ ID NO: 324 and SEQ ID NO: 481, respectively;
xi. SEQ ID NO: 325 and SEQ ID NO: 482, respectively;
xii. SEQ ID NO: 326 and SEQ ID NO: 483, respectively;
xiii. SEQ ID NO: 327 and SEQ ID NO: 484, respectively;
xiv. SEQ ID NO: 328 and SEQ ID NO: 485, respectively;
xv. SEQ ID NO: 329 and SEQ ID NO: 486, respectively;
xvi. SEQ ID NO: 330 and SEQ ID NO: 487, respectively;
xvii. SEQ ID NO: 331 and SEQ ID NO: 488, respectively;
xviii. SEQ ID NO: 332 and SEQ ID NO: 489, respectively;
xix. SEQ ID NO: 333 and SEQ ID NO: 490, respectively;
xx. SEQ ID NO: 334 and SEQ ID NO: 491, respectively;
xxi. SEQ ID NO: 335 and SEQ ID NO: 492, respectively;
xxii. SEQ ID NO: 336 and SEQ ID NO: 493, respectively;
xxiii. SEQ ID NO: 337 and SEQ ID NO: 494, respectively;
xxiv. SEQ ID NO: 338 and SEQ ID NO: 495, respectively;
xxv. SEQ ID NO: 339 and SEQ ID NO: 496, respectively;
xxvi. SEQ ID NO: 340 and SEQ ID NO: 497, respectively;
xxvii. SEQ ID NO: 341 and SEQ ID NO: 498, respectively;
xxviii. SEQ ID NO: 342 and SEQ ID NO: 499, respectively;
xxix. SEQ ID NO: 343 and SEQ ID NO: 500, respectively;
xxx. SEQ ID NO: 344 and SEQ ID NO: 501, respectively;
xxxi. SEQ ID NO: 345 and SEQ ID NO: 502, respectively;
xxxii. SEQ ID NO: 346 and SEQ ID NO: 503, respectively;
xxxiii. SEQ ID NO: 347 and SEQ ID NO: 504, respectively;
xxxiv. SEQ ID NO: 348 and SEQ ID NO: 505, respectively;
xxxv. SEQ ID NO: 349 and SEQ ID NO: 506, respectively;
xxxvi. SEQ ID NO: 350 and SEQ ID NO: 507, respectively;
xxxvii. SEQ ID NO: 351 and SEQ ID NO: 508, respectively;
xxxviii. SEQ ID NO: 352 and SEQ ID NO: 509, respectively;
xxxix. SEQ ID NO: 353 and SEQ ID NO: 510, respectively;
xl. SEQ ID NO: 354 and SEQ ID NO: 511, respectively;
xli. SEQ ID NO: 355 and SEQ ID NO: 512, respectively;
xlii. SEQ ID NO: 356 and SEQ ID NO: 513, respectively;
xliii. SEQ ID NO: 357 and SEQ ID NO: 514, respectively;
xliv. SEQ ID NO: 358 and SEQ ID NO: 515, respectively;
xlv. SEQ ID NO: 359 and SEQ ID NO: 516, respectively;
xlvi. SEQ ID NO: 360 and SEQ ID NO: 517, respectively;
xlvii. SEQ ID NO: 361 and SEQ ID NO: 518, respectively;
xlviii. SEQ ID NO: 362 and SEQ ID NO: 519, respectively;
xlix. SEQ ID NO: 363 and SEQ ID NO: 520, respectively;
l. SEQ ID NO: 364 and SEQ ID NO: 521, respectively;
li. SEQ ID NO: 365 and SEQ ID NO: 522, respectively;
lii. SEQ ID NO: 366 and SEQ ID NO: 523, respectively;
liii. SEQ ID NO: 367 and SEQ ID NO: 524, respectively;
liv. SEQ ID NO: 368 and SEQ ID NO: 525, respectively;
lv. SEQ ID NO: 369 and SEQ ID NO: 526, respectively;
lvi. SEQ ID NO: 370 and SEQ ID NO: 527, respectively;
lvii. SEQ ID NO: 371 and SEQ ID NO: 528, respectively;
lviii. SEQ ID NO: 372 and SEQ ID NO: 529, respectively;
lix. SEQ ID NO: 373 and SEQ ID NO: 530, respectively;
lx. SEQ ID NO: 374 and SEQ ID NO: 531, respectively;
lxi. SEQ ID NO: 375 and SEQ ID NO: 532, respectively;
lxii. SEQ ID NO: 376 and SEQ ID NO: 533, respectively;
lxiii. SEQ ID NO: 377 and SEQ ID NO: 534, respectively;
lxiv. SEQ ID NO: 378 and SEQ ID NO: 535, respectively;
lxv. SEQ ID NO: 379 and SEQ ID NO: 536, respectively;
lxvi. SEQ ID NO: 380 and SEQ ID NO: 537, respectively;
lxvii. SEQ ID NO: 381 and SEQ ID NO: 538, respectively;
lxviii. SEQ ID NO: 382 and SEQ ID NO: 539, respectively;
lxix. SEQ ID NO: 383 and SEQ ID NO: 540, respectively;
lxx. SEQ ID NO: 384 and SEQ ID NO: 541, respectively;
lxxi. SEQ ID NO: 385 and SEQ ID NO: 542, respectively;
lxxii. SEQ ID NO: 386 and SEQ ID NO: 543, respectively;
lxxiii. SEQ ID NO: 387 and SEQ ID NO: 544, respectively;
lxxiv. SEQ ID NO: 388 and SEQ ID NO: 545, respectively;
lxxv. SEQ ID NO: 389 and SEQ ID NO: 546, respectively;
lxxvi. SEQ ID NO: 390 and SEQ ID NO: 547, respectively;
lxxvii. SEQ ID NO: 391 and SEQ ID NO: 548, respectively;
lxxviii. SEQ ID NO: 392 and SEQ ID NO: 549, respectively;
lxxix. SEQ ID NO: 393 and SEQ ID NO: 550, respectively;
lxxx. SEQ ID NO: 394 and SEQ ID NO: 551, respectively;
lxxxi. SEQ ID NO: 395 and SEQ ID NO: 552, respectively;
lxxxii. SEQ ID NO: 396 and SEQ ID NO: 553, respectively;
lxxxiii. SEQ ID NO: 397 and SEQ ID NO: 554, respectively;
lxxxiv. SEQ ID NO: 398 and SEQ ID NO: 555, respectively;
lxxxv. SEQ ID NO: 399 and SEQ ID NO: 556, respectively;
lxxxvi. SEQ ID NO: 400 and SEQ ID NO: 557, respectively;
lxxxvii. SEQ ID NO: 401 and SEQ ID NO: 558, respectively;
lxxxviii. SEQ ID NO: 402 and SEQ ID NO: 559, respectively;
lxxxix. SEQ ID NO: 403 and SEQ ID NO: 560, respectively;
xc. SEQ ID NO: 404 and SEQ ID NO: 561, respectively;
xci. SEQ ID NO: 405 and SEQ ID NO: 562, respectively;
xcii. SEQ ID NO: 406 and SEQ ID NO: 563, respectively;
xciii. SEQ ID NO: 407 and SEQ ID NO: 564, respectively;
xciv. SEQ ID NO: 408 and SEQ ID NO: 565, respectively;
xcv. SEQ ID NO: 409 and SEQ ID NO: 566, respectively;
xcvi. SEQ ID NO: 410 and SEQ ID NO: 567, respectively;
xcvii. SEQ ID NO: 411 and SEQ ID NO: 568, respectively;
xcviii. SEQ ID NO: 412 and SEQ ID NO: 569, respectively;
xcix. SEQ ID NO: 413 and SEQ ID NO: 570, respectively;
c. SEQ ID NO: 414 and SEQ ID NO: 571, respectively;
ci. SEQ ID NO: 415 and SEQ ID NO: 572, respectively;
cii. SEQ ID NO: 416 and SEQ ID NO: 573, respectively;
ciii. SEQ ID NO: 417 and SEQ ID NO: 574, respectively;
civ. SEQ ID NO: 418 and SEQ ID NO: 575, respectively;
cv. SEQ ID NO: 419 and SEQ ID NO: 576, respectively;
cvi. SEQ ID NO: 420 and SEQ ID NO: 577, respectively;
cvii. SEQ ID NO: 421 and SEQ ID NO: 578, respectively;
cviii. SEQ ID NO: 422 and SEQ ID NO: 579, respectively;
cix. SEQ ID NO: 423 and SEQ ID NO: 580, respectively;
cx. SEQ ID NO: 424 and SEQ ID NO: 581, respectively;
cxi. SEQ ID NO: 425 and SEQ ID NO: 582, respectively;
cxii. SEQ ID NO: 426 and SEQ ID NO: 583, respectively;
cxiii. SEQ ID NO: 427 and SEQ ID NO: 584, respectively;
cxiv. SEQ ID NO: 428 and SEQ ID NO: 585, respectively;
cxv. SEQ ID NO: 429 and SEQ ID NO: 586, respectively;
cxvi. SEQ ID NO: 430 and SEQ ID NO: 587, respectively;
cxvii. SEQ ID NO: 431 and SEQ ID NO: 588, respectively;
cxviii. SEQ ID NO: 432 and SEQ ID NO: 589, respectively;
cxix. SEQ ID NO: 433 and SEQ ID NO: 590, respectively;
cxx. SEQ ID NO: 434 and SEQ ID NO: 591, respectively;
cxxi. SEQ ID NO: 435 and SEQ ID NO: 592, respectively;
cxxii. SEQ ID NO: 436 and SEQ ID NO: 593, respectively;
cxxiii. SEQ ID NO: 437 and SEQ ID NO: 594, respectively;
cxxiv. SEQ ID NO: 438 and SEQ ID NO: 595, respectively;
cxxv. SEQ ID NO: 439 and SEQ ID NO: 596, respectively;
cxxvi. SEQ ID NO: 440 and SEQ ID NO: 597, respectively;
cxxvii. SEQ ID NO: 441 and SEQ ID NO: 598, respectively;
cxxviii. SEQ ID NO: 442 and SEQ ID NO: 599, respectively;
cxxix. SEQ ID NO: 443 and SEQ ID NO: 600, respectively;
cxxx. SEQ ID NO: 444 and SEQ ID NO: 601, respectively;
cxxxi. SEQ ID NO: 445 and SEQ ID NO: 602, respectively;
cxxxii. SEQ ID NO: 446 and SEQ ID NO: 603, respectively;
cxxxiii. SEQ ID NO: 447 and SEQ ID NO: 604, respectively;
cxxxiv. SEQ ID NO: 448 and SEQ ID NO: 605, respectively;
cxxxv. SEQ ID NO: 449 and SEQ ID NO: 606, respectively;
cxxxvi. SEQ ID NO: 450 and SEQ ID NO: 607, respectively;
cxxxvii. SEQ ID NO: 451 and SEQ ID NO: 608, respectively;
cxxxviii. SEQ ID NO: 452 and SEQ ID NO: 609, respectively;
cxxxix. SEQ ID NO: 453 and SEQ ID NO: 610, respectively;
cxl. SEQ ID NO: 454 and SEQ ID NO: 611, respectively;
cxli. SEQ ID NO: 455 and SEQ ID NO: 612, respectively;
cxlii. SEQ ID NO: 456 and SEQ ID NO: 613, respectively;
cxliii. SEQ ID NO: 457 and SEQ ID NO: 614, respectively;
cxliv. SEQ ID NO: 458 and SEQ ID NO: 615, respectively;
cxlv. SEQ ID NO: 459 and SEQ ID NO: 616, respectively;
cxlvi. SEQ ID NO: 460 and SEQ ID NO: 617, respectively;
cxlvii. SEQ ID NO: 461 and SEQ ID NO: 618, respectively;
cxlviii. SEQ ID NO: 462 and SEQ ID NO: 619, respectively;
cxlix. SEQ ID NO: 463 and SEQ ID NO: 620, respectively;
cl. SEQ ID NO: 464 and SEQ ID NO: 621, respectively;
cli. SEQ ID NO: 465 and SEQ ID NO: 622, respectively;
clii. SEQ ID NO: 466 and SEQ ID NO: 623, respectively;
cliii. SEQ ID NO: 467 and SEQ ID NO: 624, respectively;
cliv. SEQ ID NO: 468 and SEQ ID NO: 625, respectively;
clv. SEQ ID NO: 469 and SEQ ID NO: 626, respectively;
clvi. SEQ ID NO: 470 and SEQ ID NO: 627, respectively; and
clvii. SEQ ID NO: 471 and SEQ ID NO: 628, respectively,
wherein the antibody comprises one or more cysteine amino acid substitution(s) at one or more position(s) selected from 88 of the light chain, 384 of the heavy chain, or 487 of the heavy chain, according to AHo numbering.

E194. The molecule of E193, wherein:
the light chain comprises the amino acid sequence of SEQ ID NO: 388 and the heavy chain comprises the amino acid sequence of SEQ ID NO: 1571; or
the light chain comprises the amino acid sequence of SEQ ID NO: 455 and the heavy chain comprises the amino acid sequence of SEQ ID NO: 1572; or
the light chain comprises the amino acid sequence of SEQ ID NO: 389 and the heavy chain comprises the amino acid sequence of SEQ ID NO: 1573; or
the light chain comprises the amino acid sequence of SEQ ID NO: 455 and the heavy chain comprises the amino acid sequence of SEQ ID NO: 1574; or
the light chain comprises the amino acid sequence of SEQ ID NO: 1575 and the heavy chain comprises the amino acid sequence of SEQ ID NO: 612,
preferably wherein the light chain comprises the amino acid sequence of SEQ ID NO: 388 and the heavy chain comprises the amino acid sequence of SEQ ID NO: 1571.

E195. A molecule comprising:
a first polypeptide that agonizes a glucagon receptor (“GCGR”);
a first linker polypeptide;
an antibody that specifically binds to a glucose-dependent insulinotropic polypeptide receptor (“GIPR”);
a second linker polypeptide; and
a second polypeptide that agonizes a GCGR,
wherein:
   an ε-amino group of a lysine residue of the first polypeptide is covalently linked to a C-terminus of the first linker polypeptide;
   an N-terminus of the first linker polypeptide is conjugated to a cysteine residue of the antibody;
   an ε-amino group of a lysine residue of the second polypeptide is covalently linked to a C-terminus of the second linker polypeptide; and
   an N-terminus of the second linker polypeptide is conjugated to a cysteine residue of the antibody.

E196. The molecule of E195, wherein the first linker polypeptide and the second linker polypeptide independently comprise amino acid sequences selected from SEQ ID NOs: 1628-1683, preferably SEQ ID NOs: 1628-1630, preferably SEQ ID NO: 1630.

E197. The molecule of E195 or E196, wherein the first polypeptide and the second polypeptide independently comprise amino acid sequences selected from SEQ ID NOs: 1587, 1592, 1596, 1615, and 1626.

E198. The molecule of any one of E195-E197, wherein the first polypeptide has the same amino acid sequence as the second polypeptide.

E199. The molecule of any one of E195-E198, wherein the first linker polypeptide has the same amino acid sequence as the second linker polypeptide.

E200. The molecule of any one of E195-E199, wherein:
the first polypeptide has the same amino acid sequence as the second polypeptide; and
the first linker polypeptide has the same amino acid sequence as the second linker polypeptide.

E201. The molecule of any one of E195-E200, wherein the lysine residue of the first polypeptide comprising the ε-amino group that is covalently linked to the C-terminus of the first linker polypeptide is at position 24 or position 28.

E202. The molecule of any one of E195-E201, wherein the lysine residue of the second polypeptide comprising the ε-amino group that is covalently linked to the C-terminus of the second linker polypeptide is at position 24 or position 28.

E203. The molecule of any one of E195-E202, wherein:
the lysine residue of the first polypeptide comprising the ε-amino group that is covalently linked to the C-terminus of the first linker polypeptide is at position 28; and
the lysine residue of the second polypeptide comprising the ε-amino group that is covalently linked to the C-terminus of the second linker polypeptide is at position 28.

E204. The molecule of any one of E195-E203, wherein the cysteine residues of the antibody that are conjugated to the N-termini of the first linker polypeptide and the second linker polypeptide are at positions selected from the group consisting of 88 of both light chains, 384 of both heavy chains, and 487 of both heavy chains, according to AHo numbering.

E205. The molecule of E204, wherein the cysteine residues of the antibody that are conjugated to the N-termini of the first linker polypeptide and the second linker polypeptide are at positions 384 of both heavy chains, according to AHo numbering.

E206. The molecule of any one of E195-E205, wherein:
the lysine residue of the first polypeptide comprising the ε-amino group that is covalently linked to the C-terminus of the first linker polypeptide is at position 28;
the lysine residue of the second polypeptide comprising the ε-amino group that is covalently linked to the C-terminus of the second linker polypeptide is at position 28; and
the cysteine residues of the antibody that are conjugated to the N-termini of the first linker polypeptide and the second linker polypeptide are at positions 384 of both heavy chains, according to AHo numbering.

E207. The molecule of any one of E195-E206, wherein the antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise the amino acid sequences of SEQ ID NO: 702, SEQ ID NO: 859, SEQ ID NO: 1016, SEQ ID NO: 1173, SEQ ID NO: 1330, and SEQ ID NO: 1487, respectively.

E208. The molecule of any one of E195-E207, wherein the antibody comprises a light chain variable region comprising the amino acid sequence of SEQ ID NO: 74 and a heavy chain variable region comprising the amino acid sequence of SEQ ID NO: 231.

E209. The molecule of any one of E195-E208, wherein the antibody comprises a light chain comprising the amino acid sequence of SEQ ID NO: 388 and a heavy chain comprising the amino acid sequence of SEQ ID NO: 1571.

E210. A molecule comprising:
a polypeptide that agonizes a glucagon receptor (“GCGR”) comprising the amino acid sequence of SEQ ID NO: 1615;
a linker polypeptide comprising the amino acid sequence of SEQ ID NO: 1629; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

E211. A molecule comprising:
a polypeptide that agonizes a glucagon receptor (“GCGR”) comprising the amino acid sequence of SEQ ID NO: 1626;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

E212. A molecule comprising:
a polypeptide that agonizes a glucagon receptor (“GCGR”) comprising the amino acid sequence of SEQ ID NO: 1592;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 24 of the polypeptide is covalently linked to a C-terminus of the linker polypeptide; and
   an N-terminus of the linker polypeptide is conjugated to a cysteine residue at position 275 of the heavy chain of the antibody.

E213. A molecule comprising:
a polypeptide that agonizes a glucagon receptor (“GCGR”) comprising the amino acid sequence of SEQ ID NO: 1626;
a linker polypeptide comprising the amino acid sequence of SEQ ID NO: 1629; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

E214. A molecule comprising:
a polypeptide that agonizes a glucagon receptor (“GCGR”) comprising the amino acid sequence of SEQ ID NO: 1587;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

E215. A molecule comprising:
a polypeptide that agonizes a glucagon receptor (“GCGR”) comprising the amino acid sequence of SEQ ID NO: 1587;
a linker polypeptide comprising the amino acid sequence of SEQ ID NO: 1630; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

E216. A molecule comprising:
a first polypeptide and a second polypeptide that agonize a glucagon receptor (“GCGR”), wherein the first polypeptide and the second polypeptide each comprise the amino acid sequence of SEQ ID NO: 1615;
a first linker polypeptide and a second linker polypeptide, wherein the first linker polypeptide and the second linker polypeptide each comprise the amino acid sequence of SEQ ID NO: 1629; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

E217. A molecule comprising:
a first polypeptide and a second polypeptide that agonize a glucagon receptor (“GCGR”), wherein the first polypeptide and the second polypeptide each comprise the amino acid sequence of SEQ ID NO: 1626;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

E218. A molecule comprising:
a first polypeptide and a second polypeptide that agonize a glucagon receptor (“GCGR”), wherein the first polypeptide and the second polypeptide each comprise the amino acid sequence of SEQ ID NO: 1592;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 24 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 24 of the second polypeptide is covalently linked to a C-terminus of the second linker polypeptide; and
   an N-terminus of the second linker polypeptide is conjugated to a cysteine residue at position 275 of the second heavy chain of the antibody.

E219. A molecule comprising:
a first polypeptide and a second polypeptide that agonize a glucagon receptor (“GCGR”), wherein the first polypeptide and the second polypeptide each comprise the amino acid sequence of SEQ ID NO: 1626;
a first linker polypeptide and a second linker polypeptide, wherein the first linker polypeptide and the second linker polypeptide each comprise the amino acid sequence of SEQ ID NO: 1629; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

E220. A molecule comprising:
a first polypeptide and a second polypeptide that agonize a glucagon receptor (“GCGR”), wherein the first polypeptide and the second polypeptide each comprise the amino acid sequence of SEQ ID NO: 1587;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

E221. A molecule comprising:
a first polypeptide and a second polypeptide that agonize a glucagon receptor (“GCGR”), wherein the first polypeptide and the second polypeptide each comprise the amino acid sequence of SEQ ID NO: 1587;
a first linker polypeptide and a second linker polypeptide, wherein the first linker polypeptide and the second linker polypeptide each comprise the amino acid sequence of SEQ ID NO: 1630; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

E222. A pharmaceutical composition comprising:
a polypeptide of any one of E1-E108 or a molecule of any one of E109-E221; and
a pharmaceutically acceptable excipient.

E223. A method of treating obesity in a subject in need thereof, the method comprising administering a polypeptide of any one of E1-E108, a molecule of any one of E109-E221, or a pharmaceutical composition of E222 to the subject.

E224. A polypeptide of any one of E1-E108, a molecule of any one of E109-E221, or a pharmaceutical composition of E222 for use in treating obesity.

E225. Use of a polypeptide of any one of E1-E108 or a molecule of any one of E109-E221 in the manufacture of a medicament for treating obesity.

E226. The method of E223, the polypeptide, molecule or pharmaceutical composition for use of E224, and the use of E225, wherein treating obesity comprises lowering blood glucose, insulin, triglyceride, or cholesterol levels; reducing body weight; and/or improving glucose tolerance, energy expenditure, or insulin sensitivity.

E227. A method of reducing body weight and/or food intake in a subject in need thereof, the method comprising administering a polypeptide of any one of E1-E108, a molecule of any one of E109-E221, or a pharmaceutical composition of E222 to the subject.

E228. A polypeptide of any one of E1-E108, a molecule of any one of E109-E221, or a pharmaceutical composition of E222 for use in reducing body weight and/or food intake in a subject in need thereof.

E229. Use of a polypeptide of any one of E1-E108 or a molecule of any one of E109-E221 in the manufacture of a medicament for reducing body weight and/or food intake in a subject in need thereof.

E230. The method of E227, the polypeptide, molecule or pharmaceutical composition for use of E228, and the use of E229, wherein the subject is overweight or obese.

E231. A method of treating obesity in a subject in need thereof, the method comprising administering a polypeptide of any one of E1-E108, a molecule of any one of E109-E221, or a pharmaceutical composition of E222 to the subject in combination with a GLP-1 agonist.

E232. A polypeptide of any one of E1-E108, a molecule of any one of E109-E221, or a pharmaceutical composition of E222 for use in a method of treating obesity in a subject in need thereof, wherein the method comprises administering the polypeptide, molecule, or pharmaceutical composition to the subject in combination with a GLP-1 agonist.

E233. The method of E231 or the polypeptide, molecule or pharmaceutical composition for use of E232, wherein treating obesity comprises lowering blood glucose, insulin, triglyceride, or cholesterol levels; reducing body weight; and/or improving glucose tolerance, energy expenditure, or insulin sensitivity.

E234. The method of E231 or the polypeptide, molecule or pharmaceutical composition for use of E232, wherein, prior to the administering, the subject experienced a weight-loss plateau.

E235. The method of E231 or the polypeptide, molecule or pharmaceutical composition for use of E232, wherein the GLP-1 agonist is semaglutide.

E236. A method of reducing body weight and/or food intake in a subject in need thereof, the method comprising administering a polypeptide of any one of E1-E108, a molecule of any one of E109-E221, or a pharmaceutical composition of E222 to the subject in combination with a GLP-1 agonist.

E237. A polypeptide of any one of E1-E108, a molecule of any one of E109-E221, or a pharmaceutical composition of E222 for use in a method of reducing body weight and/or food intake in a subject in need thereof, wherein the method comprises administering the polypeptide, molecule, or pharmaceutical composition to the subject in combination with a GLP-1 agonist.

E238. The method of E236 or the polypeptide, molecule, or pharmaceutical composition for use of E237, wherein the subject is overweight or obese.

E239. The method of E236 or the polypeptide, molecule, or pharmaceutical composition for use of E237, wherein the GLP-1 agonist is semaglutide.

E240. The method of E236 or the polypeptide, molecule, or pharmaceutical composition for use of E237, wherein the subject is overweight or obese and the GLP-1 agonist is semaglutide.

E241. The method of any one of E236 or E238-E240 or the polypeptide, molecule, or pharmaceutical composition for use of any one of E237-E240, wherein the GLP-1 agonist and the polypeptide, molecule, or pharmaceutical composition for use are administered in consecutive, non-overlapping dosing intervals, wherein the GLP-1 agonist is administered prior to the polypeptide, molecule, or pharmaceutical composition.

E242. The method of any one of E236 or E238-E240 or the polypeptide, molecule, or pharmaceutical composition for use of any one of E237-E240, wherein the GLP-1 agonist and the polypeptide, molecule, or pharmaceutical composition for use are administered concurrently.

E243. The method or the polypeptide, molecule, or pharmaceutical composition for use of E242, wherein at least one dose of the GLP-1 agonist is administered prior to a first administration of the polypeptide, molecule, or pharmaceutical composition.

Further non-limiting example embodiments/features of the present disclosure include:

F1. A polypeptide that agonizes a glucagon receptor (“GCGR”), wherein the polypeptide comprises an amino acid sequence with between three and nine modifications relative to SEQ ID NO: 1576, wherein the modifications are selected from:
tyrosine and phenylalanine at position 1;
d-serine, 2-aminoisobutyric acid, and d-threonine at position 2;
glutamic acid at position 3;
histidine at position 7;
tryptophan at position 10;
glutamic acid at position 15;
2-aminoisobutyric acid, glutamine, homophenylalanine, and glutamic acid at position 16;
lysine, citrulline, glutamine, and alanine at position 17;
2-naphthylalanine, L-4,4′-biphenylalanine, alanine, citrulline, and lysine at position 18;
4-chloro-L-phenylalanine, alanine, d-glutamine, homoserine, histidine, arginine, and glutamic acid at position 20;
glutamic acid, citrulline, and d-aspartic acid at position 21;
tryptophan and β-cyclohexyl-L-alanine at position 22;
aspartic acid, lysine, alanine, 2-aminoisobutyric acid, glycine, histidine, asparagine, threonine, d-glutamine, glutamic acid, arginine, phenylalanine, leucine, serine, tyrosine, valine, isoleucine, homoserine, and 2,3-diaminopropionic acid at position 24;
5-bromo-L-tryptophan, tyrosine, L-beta-homotryptophan, 5-methoxy-L-tryptophan, 5-methyl-L-tryptophan, 6-bromo-L-tryptophan, 6-chloro-L-tryptophan, 6-methyl-L-tryptophan, and 7-bromo-L-tryptophan at position 25;
leucine, glutamic acid, and L-α-aminoadipic acid at position 27;
lysine, aspartic acid, serine, 6-azido-L-lysine, glutamic acid, and alanine at position 28;
glutamic acid, serine, aspartic acid, and alanine at position 29;
an additional amino acid at position 30, wherein the additional amino acid is lysine; and
an additional amino acid at position 31, wherein the additional amino acid is lysine.

F2. The polypeptide of F1, wherein the modifications are selected from: tyrosine and phenylalanine at position 1;
d-serine, 2-aminoisobutyric acid, and d-threonine at position 2;
glutamic acid at position 3;
histidine at position 7;
tryptophan at position 10;
glutamic acid at position 15;
2-aminoisobutyric acid, glutamine, homophenylalanine, and glutamic acid at position 16;
lysine, citrulline, glutamine, and alanine at position 17;
2-naphthylalanine, L-4,4′-biphenylalanine, alanine, citrulline, and lysine at position 18;
4-chloro-L-phenylalanine, alanine, d-glutamine, homoserine, histidine, arginine, and glutamic acid at position 20;
glutamic acid, citrulline, and d-aspartic acid at position 21;
tryptophan and β-cyclohexyl-L-alanine at position 22;
aspartic acid, lysine, alanine, 2-aminoisobutyric acid, glycine, histidine, asparagine, threonine, d-glutamine, glutamic acid, arginine, phenylalanine, leucine, serine, tyrosine, valine, isoleucine, homoserine, and 2,3-diaminopropionic acid at position 24;
5-bromo-L-tryptophan, tyrosine, L-beta-homotryptophan, 5-methoxy-L-tryptophan, 5-methyl-L-tryptophan, 6-bromo-L-tryptophan, 6-chloro-L-tryptophan, 6-methyl-L-tryptophan, and 7-bromo-L-tryptophan at position 25;
leucine, glutamic acid, and L-α-aminoadipic acid at position 27;
lysine, aspartic acid, serine, 6-azido-L-lysine, glutamic acid, and alanine at position 28;
glutamic acid, serine, aspartic acid, and alanine at position 29.

F3. The polypeptide of F1 or F2, wherein the modifications comprise d-serine at position 2.

F4. The polypeptide of any one of F1 to F3, wherein the modifications comprise 2-aminoisobutyric acid at position 16.

F5. The polypeptide of any one of F1 to F4, wherein the modifications comprise d-serine at position 2 and 2-aminoisobutyric acid at position 16.

F6. The polypeptide of any one of F1 to F5, wherein the modifications comprise lysine at position 17.

F7. The polypeptide of any one of F1 to F6, wherein the modifications comprise glutamic acid at position 21.

F8. The polypeptide of any one of F1 to F7, wherein the modifications comprise lysine, alanine, or glutamic acid at position 24.

F9. The polypeptide of any one of F1 to F8, wherein the modifications comprise 5-bromo-L-tryptophan at position 25.

F10. The polypeptide of any one of F1 to F9, wherein the modifications comprise leucine or glutamic acid at position 27.

F11. The polypeptide of any one of F1 to F10, wherein the modifications comprise lysine, aspartic acid, or glutamic acid at position 28.

F12. The polypeptide of any one of F1 to F11, wherein the modifications comprise glutamic acid or serine at position 29.

F13. The polypeptide of any one of F1 to F12, wherein the modifications comprise lysine at position 24 or position 28.

F14. The polypeptide of any one of F1 to F13, wherein the polypeptide comprises 29 amino acids.

F15. The polypeptide of any one of F1 or F3 to F13, wherein the polypeptide comprises 31 amino acids, and further wherein the polypeptide comprises lysine at position 30 and lysine at position 31.

F16. The polypeptide of F1, wherein the modifications comprise d-serine at position 2, 2-aminoisobutyric acid at position 16, and between one and seven other modifications selected from:
lysine at position 17;
glutamic acid at position 21;
lysine, alanine, and glutamic acid at position 24;
5-bromo-L-tryptophan at position 25;
leucine and glutamic acid at position 27;
lysine, aspartic acid, and glutamic acid at position 28; and
glutamic acid and serine at position 29.

F17. The polypeptide of F1 or F16, wherein the modifications comprise d-serine at position 2, 2-aminoisobutyric acid at position 16, and between one and six other modifications selected from:
lysine at position 17;
glutamic acid at position 21;
lysine, alanine, and glutamic acid at position 24;
leucine and glutamic acid at position 27;
lysine, aspartic acid, and glutamic acid at position 28; and
glutamic acid and serine at position 29.

F18. The polypeptide of F1, F16, or F17, wherein the modifications comprise d-serine at position 2, 2-aminoisobutyric acid at position 16, and one or two other modifications selected from:
lysine at position 24; and
lysine and glutamic acid at position 28.

F19. The polypeptide of any one of F16 to F18, wherein the polypeptide comprises 29 amino acids.

F20. A polypeptide that agonizes a glucagon receptor (“GCGR”), wherein the polypeptide comprises at least 28 amino acids, wherein the polypeptide comprises a d-serine at position 2 and a 2-aminoisobutyric acid at position 16; and the polypeptide has at least 89% (e.g., at least 93%, at least 96%, or 100%) sequence identity to the amino acid sequence of SEQ ID NO: 1822.

F21. The polypeptide of F20, wherein the polypeptide has at least 93% sequence identity to the amino acid sequence of SEQ ID NO: 93.

F22. The polypeptide of F20 or F21, wherein the polypeptide comprises the amino acid sequence of SEQ ID NO: 1822.

F23. The polypeptide of F20, F21, or F22, wherein the polypeptide comprises 29 amino acids.

F24. The polypeptide of any one of F20 to F23, wherein the polypeptide consists of the amino acid sequence of SEQ ID NO: 1822.

F25. A polypeptide that agonizes a glucagon receptor (“GCGR”), wherein the polypeptide comprises the amino acid sequence of any one of SEQ ID NOs: 1587-1627, 1747-1840, 1859-1862, or 1879-1881.

F26. The polypeptide of F25, wherein the polypeptide comprises the amino acid sequence of any one of SEQ ID NOs: 1587-1627 or 1747.

F27. The polypeptide of F25, wherein the polypeptide comprises the amino acid sequence of any one of SEQ ID NOs: 1747-1840, 1859-1862, or 1879-1881.

F28. The polypeptide of F25, wherein the polypeptide comprises the amino acid sequence of any one of SEQ ID NOs: 1596, 1597, 1615, 1626, 1818, 1822, 1825, or 1826.

F29. The polypeptide of F25, wherein the polypeptide comprises the amino acid sequence of any one of SEQ ID NOs: 1587, 1592, 1596, 1615, or 1626.

F30. The polypeptide of F25, wherein the polypeptide comprises the amino acid sequence of any one of SEQ ID NOs: 1626, 1818, 1822, 1825, or 1826.

F31. The polypeptide of F25, wherein the polypeptide comprises the amino acid sequence of SEQ ID NO: 1587.

F32. The polypeptide of F25 or F31, wherein the polypeptide consists of the amino acid sequence of SEQ ID NO: 1587.

F33. The polypeptide of F25, wherein the polypeptide comprises the amino acid sequence of SEQ ID NO: 1592.

F34. The polypeptide of F25 or F33, wherein the polypeptide consists of the amino acid sequence of SEQ ID NO: 1592.

F35. The polypeptide of F25, wherein the polypeptide comprises the amino acid sequence of SEQ ID NO: 1596.

F36. The polypeptide of F25 or F35, wherein the polypeptide consists of the amino acid sequence of SEQ ID NO: 1596.

F37. The polypeptide of F25, wherein the polypeptide comprises the amino acid sequence of SEQ ID NO: 1597.

F38. The polypeptide of F25 or F37, wherein the polypeptide consists of the amino acid sequence of SEQ ID NO: 1597.

F39. The polypeptide of F25, wherein the polypeptide comprises the amino acid sequence of SEQ ID NO: 1615.

F40. The polypeptide of F25 or F39, wherein the polypeptide consists of the amino acid sequence of SEQ ID NO: 1615.

F41. The polypeptide of F25, wherein the polypeptide comprises the amino acid sequence of SEQ ID NO: 1626.

F42. The polypeptide of F25 or F41, wherein the polypeptide consists of the amino acid sequence of SEQ ID NO: 1626.

F43. The polypeptide of F25, wherein the polypeptide comprises the amino acid sequence of SEQ ID NO: 1818.

F44. The polypeptide of F25 or F43, wherein the polypeptide consists of the amino acid sequence of SEQ ID NO: 1818.

F45. The polypeptide of F25, wherein the polypeptide comprises the amino acid sequence of SEQ ID NO: 1822.

F46. The polypeptide of F25 or F45, wherein the polypeptide consists of the amino acid sequence of SEQ ID NO: 1822.

F47. The polypeptide of F25, wherein the polypeptide comprises the amino acid sequence of SEQ ID NO: 1825.

F48. The polypeptide of F25 or F47, wherein the polypeptide consists of the amino acid sequence of SEQ ID NO: 1825.

F49. The polypeptide of F25, wherein the polypeptide comprises the amino acid sequence of SEQ ID NO: 1826.

F50. The polypeptide of F25 or F49, wherein the polypeptide consists of the amino acid sequence of SEQ ID NO: 1826.

F51. The polypeptide of F25 or F26, wherein the polypeptide consists of the amino acid sequence of any one of SEQ ID NOs: 1587-1627 or 1747.

F52. The polypeptide of F25 or F27, wherein the polypeptide consists of the amino acid sequence of any one of SEQ ID NOs: 1747-1840, 1859-1862, or 1879-1881.

F53. The polypeptide of F25 or F28, wherein the polypeptide consists of the amino acid sequence of any one of SEQ ID NOs: 1596, 1597, 1615, 1626, 1818, 1822, 1825, or 1826.

F54. The polypeptide of F25 or F29, wherein the polypeptide consists of the amino acid sequence of any one of SEQ ID NOs: 1587, 1592, 1596, 1615, or 1626.

F55. The polypeptide of F25 or F30, wherein the polypeptide consists of the amino acid sequence of any one of SEQ ID NOs: 1626, 1818, 1822, 1825, or 1826.

F56. A molecule comprising:
a first polypeptide that agonizes a glucagon receptor (“GCGR”), wherein the first polypeptide is selected from the polypeptides of any one of F1-F55; and
a second polypeptide,
wherein the C-terminus of the second polypeptide is covalently linked to an ε-amino group of a lysine residue of the first polypeptide.

F57. The molecule of F56, wherein the second polypeptide comprises the amino acid sequence of any one of SEQ ID NOs: 1628-1683, 1739-1746, or 1841-1852.

F58. The molecule of F56 or F57, wherein the second polypeptide comprises the amino acid sequence of any one of SEQ ID NOs: 1628-1683, 1739, 1850, or 1851.

F59. The molecule of any one of F56 to F58, wherein the second polypeptide comprises the amino acid sequence of any one of SEQ ID NOs: 1628-1630.

F60. The molecule of any one of F56 to F59, wherein the second polypeptide comprises the amino acid sequence of SEQ ID NO: 1628.

F61. The molecule of any one of F56 to F59, wherein the second polypeptide comprises the amino acid sequence of SEQ ID NO: 1629.

F62. The molecule of any one of F56 to F59, wherein the second polypeptide comprises the amino acid sequence of SEQ ID NO: 1630.

F63. The molecule of any one of F56 to F62, wherein the C-terminus of the second polypeptide is covalently linked to the ε-amino group of a lysine at position 21, position 24, position 28, or position 31 of the first polypeptide (e.g., the ε-amino group of a lysine residue at position 21, position 24, position 28, or position 31 of the first polypeptide has been condensed with a carboxyl group of the C-terminus of the second polypeptide to form an amide bond between the first polypeptide and the second polypeptide).

F64. The molecule of any one of F56 to F63, wherein the C-terminus of the second polypeptide is covalently linked to the ε-amino group of a lysine at position 24 or position 28 of the first polypeptide.

F65. The molecule of any one of F56 to F63, wherein the C-terminus of the second polypeptide is covalently linked to the ε-amino group of a lysine at position 21 of the first polypeptide.

F66. The molecule of any one of F56 to F63 or F64, wherein the C-terminus of the second polypeptide is covalently linked to the ε-amino group of a lysine at position 24 of the first polypeptide.

F67. The molecule of any one of F56 to F63 or F64, wherein the C-terminus of the second polypeptide is covalently linked to the ε-amino group of a lysine at position 28 of the first polypeptide.

F68. The molecule of any one of F56 to F63, wherein the C-terminus of the second polypeptide is covalently linked to the ε-amino group of a lysine at position 31 of the first polypeptide.

F69. The molecule of any one of F56 to F63, wherein the first polypeptide comprises the amino acid sequence of any one of SEQ ID NOs: 1587-1627, 1747-1749, 1751-1792, 1794-1798, 1801-1830, 1833-1840, 1859-1862, or 1879-1881.

F70. The molecule of F56, wherein the first polypeptide comprises an amino acid sequence selected from SEQ ID NOs: 1587-1627, 1747-1749, 1751-1792, 1794-1798, 1801-1830, 1833-1840, and 1859-1862, and the second polypeptide comprises the amino acid sequence of any one of SEQ ID NOs: 1628-1683, 1739, 1850, or 1851.

F71. The molecule of F56, wherein the first polypeptide comprises an amino acid sequence selected from SEQ ID NOs: 1587-1627, 1747-1749, 1751-1792, 1794-1798, 1801-1830, 1833-1840, and 1859-1862, and the second polypeptide comprises the amino acid sequence of any one of SEQ ID NOs: 1628, 1629, 1630, 1631, 1632, 1640, or 1644.

F72. The molecule of F56, wherein the second polypeptide comprises the amino acid sequence of SEQ ID NO: 1628, 1629, 1630, 1631, 1632, 1640, or 1644.

F73. The molecule of F56, wherein the first polypeptide comprises the amino acid sequence of SEQ ID NO: 1860, the second polypeptide comprises the amino acid sequence of any one of SEQ ID NOs: 1628-1683, 1739, 1850, or 1851, and the C-terminus of the second polypeptide is covalently linked to the ε-amino group of a lysine at position 21 of the first polypeptide.

F74. The molecule of F56, wherein the first polypeptide comprises the amino acid sequence of any one of SEQ ID NOs: 1589, 1592, 1594, 1597, 1598, 1613, 1625, 1762, 1766-1768, 1787, 1788, 1797, 1798, 1804, 1805, 1811, 1812, 1821, 1822, 1833, 1837, or 1838, the second polypeptide comprises the amino acid sequence of any one of SEQ ID NOs: 1628-1683, 1739, 1850, or 1851, and the C-terminus of the second polypeptide is covalently linked to the ε-amino group of a lysine at position 24 of the first polypeptide.

F75. The molecule of F56, wherein the first polypeptide comprises the amino acid sequence of any one of SEQ ID NOs: 1587, 1588, 1590, 1591, 1593, 1595, 1596, 1599-1612, 1614-1624, 1626, 1627, 1747-1749, 1751-1761, 1763-1765, 1769-1786, 1790-1792, 1794-1796, 1801-1803, 1806-1810, 1813-1820, 1823-1830, 1834-1836, 1839, 1840, 1859, 1861, or 1862, the second polypeptide comprises the amino acid sequence of any one of SEQ ID NOs: 1628-1683, 1739, 1850, or 1851, and the C-terminus of the second polypeptide is covalently linked to the ε-amino group of a lysine at position 28 of the first polypeptide.

F76. The molecule of F56, wherein the first polypeptide comprises the amino acid sequence of SEQ ID NO: 1789, the second polypeptide comprises the amino acid sequence of any one of SEQ ID NOs: 1628-1683, 1739, 1850, or 1851, and the C-terminus of the second polypeptide is covalently linked to the ε-amino group of a lysine at position 31 of the first polypeptide.

F77. The molecule of any one of F56 to F76, wherein the N-terminal amino acid residue of the second polypeptide is modified for coupling to a cysteine residue.

F78. The molecule of any one of F56 to F77, wherein the N-terminal amino acid residue of the second polypeptide is bromoacetylated.

F79. A molecule comprising:
a first polypeptide that agonizes a glucagon receptor (“GCGR”), wherein the first polypeptide is selected from the polypeptides of any one of F1-F55; and
a second polypeptide,
wherein the C-terminal amino acid residue of the first polypeptide is covalently linked to the N-terminal amino acid residue of the second polypeptide.

F80. The molecule of F79, wherein the second polypeptide comprises the amino acid sequence of any one of SEQ ID NOs: 1628-1683, 1739-1746, or 1841-1852.

F81. The molecule of F79 or F80, wherein the second polypeptide comprises the amino acid sequence of any one of SEQ ID NOs: 1740-1746, 1841-1849, or 1852.

F82. The molecule of any one of F79 to F81, wherein the first polypeptide comprises the amino acid sequence of any one of SEQ ID NOs: 1793, 1799, 1800, 1831, or 1832.

F83. The molecule of F79, wherein the first polypeptide comprises an amino acid sequence selected from SEQ ID NOs: 1793, 1799, 1800, 1831, and 1832, and the second polypeptide comprises the amino acid sequence of any one of SEQ ID NOs: 1740-1746, 1841-1849, or 1852.

F84. The molecule of any one of F79 to F83, wherein the C-terminal amino acid residue of the second polypeptide is modified for coupling to a cysteine residue.

F85. The molecule of any one of F79 to F84, wherein the C-terminal amino acid residue of the second polypeptide is bromoacetylated.

F86. A molecule comprising:
a polypeptide that agonizes a glucagon receptor (“GCGR”), wherein the polypeptide is selected from the polypeptides of any one of F1-F55; and
a half-life extending domain (e.g., an Fc-containing polypeptide).

F87. The molecule of F86, wherein the half-life extending domain is an Fc-containing polypeptide.

F88. The molecule of F86 or F87, wherein:
the half-life extending domain is an Fc-containing polypeptide;
   an ε-amino group of a lysine residue of the polypeptide is covalently linked to a C-terminus of a linker polypeptide; and
   an N-terminus of the linker polypeptide is conjugated to a cysteine residue of the Fc-containing polypeptide.

F89. The molecule of F86 or F87, wherein:
the half-life extending domain is an Fc-containing polypeptide;
a C-terminal amino acid residue of the polypeptide is covalently linked to an N-terminal amino acid residue of the linker polypeptide; and
a C-terminal amino acid residue of the linker polypeptide is conjugated to a cysteine residue of the Fc-containing polypeptide.

F90. The molecule of F88 or F89, wherein the linker polypeptide is a linear polypeptide.

F91. The molecule of any one of F88 to F90, wherein the linker polypeptide comprises the amino acid sequence of any one of SEQ ID NOs: 1628-1683, 1739-1746, or 1841-1852.

F92. The molecule of F88, wherein the linker polypeptide comprises the amino acid sequence of any one of SEQ ID NOs: 1628-1683, 1739, 1850, or 1851.

F93. The molecule of F88 or F92, wherein the polypeptide comprises the amino acid sequence of any one of SEQ ID NOs: 1587-1627, 1747-1749, 1751-1792, 1794-1798, 1801-1830, 1833-1840, 1859-1862, or 1879-1881.

F94. The molecule of F89, wherein the linker polypeptide comprises the amino acid sequence of any one of SEQ ID NOs: 1740-1746, 1841-1849, or 1852.

F95. The molecule of F89 or F94, wherein the polypeptide comprises the amino acid sequence of any one of SEQ ID NOs: 1793, 1799, 1800, 1831, or 1832.

F96. The molecule of any one of F88 to F91, wherein the linker polypeptide comprises the amino acid sequence of any one of SEQ ID NOs: 1628-1683.

F97. The molecule of any one of F88 to F91, wherein the linker polypeptide comprises the amino acid sequence of any one of SEQ ID NOs: 1628-1630.

F98. The molecule of any one of F88 to F91, wherein the linker polypeptide comprises the amino acid sequence of SEQ ID NO: 1628.

F99. The molecule of any one of F88 to F91, wherein the linker polypeptide comprises the amino acid sequence of SEQ ID NO: 1629.

F100. The molecule of any one of F88 to F91, wherein the linker polypeptide comprises the amino acid sequence of SEQ ID NO: 1630.

F101. The molecule of any one of F88 to F91 or F96 to F100, wherein the polypeptide comprises the amino acid sequence of any one of SEQ ID NOs: 1587-1627, 1747-1840, 1859-1862, or 1879-1881.

F102. The molecule of any one of F88 to F91 or F96 to F101, wherein the polypeptide comprises the amino acid sequence of any one of SEQ ID NOs: 1587-1627 or 1747.

F103. The molecule of any one of F88 to F91 or F96 to F102, wherein the polypeptide comprises the amino acid sequence of any one of SEQ ID NOs: 1587-1627.

F104. The molecule of any one of F88 to F91 or F96 to F103, wherein the polypeptide comprises the amino acid sequence of any one of SEQ ID NOs: 1588 or 1596-1611.

F105. The molecule of any one of F86-F92, F94, or F96-F101, wherein the polypeptide comprises the amino acid sequence of any one of SEQ ID NOs: 1587, 1592, 1615, 1626, 1818, 1822, 1825, or 1826.

F106. The molecule of any one of F86-F92, F94, or F96-F101, wherein the polypeptide comprises the amino acid sequence of any one of SEQ ID NOs: 1587, 1592, 1596, 1615, or 1626.

F107. The molecule of any one of F86-F92, F94, or F96-F101, wherein the polypeptide comprises the amino acid sequence of any one of SEQ ID NOs: 1587, 1592, 1615, or 1626.

F108. The molecule of any one of F86-F92, F94, or F96-F101, wherein the polypeptide comprises the amino acid sequence of any one of SEQ ID NOs: 1626, 1818, 1822, 1825, or 1826.

F109. The molecule of any one of F86-F92, F94, F96-F103, or F105-F107, wherein the polypeptide comprises the amino acid sequence of SEQ ID NO: 1587.

F110. The molecule of any one of F86-F92, F94, F96-F103, or F105-F107, wherein the polypeptide comprises the amino acid sequence of SEQ ID NO: 1592.

Fill. The molecule of any one of F86-F92, F94, F96-F104, or F106, wherein the polypeptide comprises the amino acid sequence of SEQ ID NO: 1596.

F112. The molecule of any one of F86-F92, F94, F96-F103, or F105-107, wherein the polypeptide comprises the amino acid sequence of SEQ ID NO: 1615.

F113. The molecule of any one of F86-F92, F94, F96-F103, or F105-F108, wherein the polypeptide comprises the amino acid sequence of SEQ ID NO: 1626.

F114. The molecule of any one of F86-F92, F94, F96-F101, F105, or F108, wherein the polypeptide comprises the amino acid sequence of SEQ ID NO: 1818.

F115. The molecule of any one of F86-F92, F94, F96-F101, F105, or F108, wherein the polypeptide comprises the amino acid sequence of SEQ ID NO: 1822.

F116. The molecule of any one of F86-F92, F94, F96-F101, F105, or F108, wherein the polypeptide comprises the amino acid sequence of SEQ ID NO: 1825.

F117. The molecule of any one of F86-F92, F94, F96-F101, F105, or F108, wherein the polypeptide comprises the amino acid sequence of SEQ ID NO: 1826.

F118. The molecule of any one of F86-F117, wherein the half-life extending domain is an antibody fragment.

F119. The molecule of any one of F86-F117, wherein the half-life extending domain is an antibody.

F120. The molecule of F119, wherein the antibody is of the IgG1-, IgG2-, or IgG4-subclass.

F121. The molecule of F119 or F120, wherein the antibody is of the IgG1- or IgG2-subclass.

F122. The molecule of any one of F119-F121, wherein the antibody specifically binds to 2,4-dinitrophenol (“DNP”).

F123. The molecule of any one of F119-F121, wherein the antibody specifically binds to a glucose-dependent insulinotropic polypeptide receptor (“GIPR”).

F124. The molecule of any one of F119-121 or F123, wherein the antibody specifically binds to human GIPR.

F125. The molecule of any one of F119-121, F123, or F124, wherein the antibody inhibits binding of GIP to the extracellular portion of human GIPR.

F126. The molecule of any one of F119-121 or F123-F125, wherein the antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise the amino acid sequences of SEQ ID NO: 702, SEQ ID NO: 859, SEQ ID NO: 1016, SEQ ID NO: 1173, SEQ ID NO: 1330, and SEQ ID NO: 1487, respectively.

F127. The molecule of any one of F119-121 or F123-F126, wherein the antibody comprises a light chain variable region comprising the amino acid sequence of SEQ ID NO: 74 and a heavy chain variable region comprising the amino acid sequence of SEQ ID NO: 231.

F128. The molecule of any one of any one of F119-121 or F123-F127, wherein the antibody comprises a light chain comprising the amino acid sequence of SEQ ID NO: 388 and a heavy chain comprising the amino acid sequence of SEQ ID NO: 1571.

F129. A molecule comprising:
a first polypeptide that agonizes a glucagon receptor (“GCGR”); and
an antibody that specifically binds to a glucose-dependent insulinotropic polypeptide receptor (“GIPR”).

F130. The molecule of F129, wherein:
an ε-amino group of a lysine residue of the first polypeptide is covalently linked to a C-terminus of a first linker polypeptide; and
an N-terminus of the first linker polypeptide is conjugated to a cysteine residue of the antibody.

F131. The molecule of F129, wherein:
a C-terminal amino acid residue of the first polypeptide is covalently linked to an N-terminal amino acid residue of a first linker polypeptide; and
a C-terminal amino acid residue of the first linker polypeptide is conjugated to a cysteine residue of the antibody.

F132. The molecule of any one of F129-F131, wherein the first polypeptide is glucagon or a glucagon analog.

F133. The molecule of any one of F129-F132, wherein the first polypeptide is selected from the polypeptides of any one of F1-F55.

F134. The molecule of any one of F129-F133, wherein the first polypeptide comprises the amino acid sequence of any one of SEQ ID NOs: 1587-1627, 1747-1840, 1859-1862, or 1879-1881.

F135. The molecule of any one of F129-F134, wherein the first polypeptide comprises the amino acid sequence of any one of SEQ ID NOs: 1587-1627 or 1747.

F136. The molecule of any one of F129-F135, wherein the first polypeptide comprises the amino acid sequence of any one of SEQ ID NOs: 1587-1627.

F137. The molecule of any one of F129-F136, wherein the first polypeptide comprises the amino acid sequence of any one of SEQ ID NOs: 1587, 1592, 1596, 1615, or 1626.

F138. The molecule of any one of F129-F134, wherein the first polypeptide comprises the amino acid sequence of any one of SEQ ID NOs: 1587, 1592, 1596, 1615, 1626, 1818, 1822, 1825, or 1826.

F139. The molecule of any one of F129-F134, wherein the first polypeptide comprises the amino acid sequence of any one of SEQ ID NOs: 1587, 1592, 1615, 1626, 1818, 1822, 1825, or 1826.

F140. The molecule of any one of F129-F134, wherein the first polypeptide comprises the amino acid sequence of any one of SEQ ID NOs: 1626, 1818, 1822, 1825, or 1826.

F141. The molecule of F134, wherein the first polypeptide comprises the amino acid sequence of SEQ ID NO: 1587.

F142. The molecule of F134, wherein the first polypeptide comprises the amino acid sequence of SEQ ID NO: 1592.

F143. The molecule of F134, wherein the first polypeptide comprises the amino acid sequence of SEQ ID NO: 1596.

F144. The molecule of F134, wherein the first polypeptide comprises the amino acid sequence of SEQ ID NO: 1615.

F145. The molecule of F134, wherein the first polypeptide comprises the amino acid sequence of SEQ ID NO: 1626.

F146. The molecule of F134, wherein the first polypeptide comprises the amino acid sequence of SEQ ID NO: 1818.

F147. The molecule of F134, wherein the first polypeptide comprises the amino acid sequence of SEQ ID NO: 1822.

F148. The molecule of F134, wherein the first polypeptide comprises the amino acid sequence of SEQ ID NO: 1825.

F149. The molecule of F134, wherein the first polypeptide comprises the amino acid sequence of SEQ ID NO: 1826.

F150. The molecule of any one of F129-F149, wherein the first linker polypeptide is a linear polypeptide.

F151. The molecule of any one of F129-F150, wherein the first linker polypeptide comprises the amino acid sequence of any one of SEQ ID NOs: 1628-1683, 1739-1746, or 1841-1852.

F152. The molecule of any one of F129-F151, wherein the first linker polypeptide comprises the amino acid sequence of any one of SEQ ID NOs: 1628-1683.

F153. The molecule of any one of F129-F152, wherein the first linker polypeptide comprises the amino acid sequence of any one of SEQ ID NOs: 1628-1630.

F154. The molecule of any one of F129-F153, wherein the first linker polypeptide comprises the amino acid sequence of SEQ ID NO: 1628.

F155. The molecule of any one of F129-F153, wherein the first linker polypeptide comprises the amino acid sequence of SEQ ID NO: 1629.

F156. The molecule of any one of F129-F153, wherein the first linker polypeptide comprises the amino acid sequence of SEQ ID NO: 1630.

F157. The molecule of any one of F129-F156, wherein the cysteine residue of the antibody that is conjugated to the first linker polypeptide is at a position selected from the group consisting of 88 of a light chain, 384 of a heavy chain, and 487 of a heavy chain, according to AHo numbering.

F158. The molecule of F157, wherein the cysteine residue of the antibody that is conjugated to the first linker polypeptide is at position 384 of a heavy chain, according to AHo numbering.

F159. The molecule of any one of F129-F158, further comprising a second polypeptide that agonizes a GCGR.

F160. The molecule of F159, wherein:
an ε-amino group of a lysine residue of the second polypeptide is covalently linked to a C-terminus of a second linker polypeptide; and
an N-terminus of the second linker polypeptide is conjugated to a cysteine residue of the antibody.

F161. The molecule of F159, wherein:
a C-terminal amino acid residue of the second polypeptide is covalently linked to an N-terminal amino acid residue of a second linker polypeptide; and
a C-terminal amino acid residue of the second linker polypeptide is conjugated to a cysteine residue of the antibody.

F162. The molecule of any one of F159-F161, wherein the second polypeptide is glucagon or a glucagon analog.

F163. The molecule of any one of F159-F162, wherein the second polypeptide is selected from the polypeptides of any one of F1-F55.

F164. The molecule of any one of F159-F163, wherein the second polypeptide comprises the amino acid sequence of any one of SEQ ID NOs: 1587-1627, 1747-1840, 1859-1862, or 1879-1881.

F165. The molecule of any one of F159-F164, wherein the second polypeptide comprises the amino acid sequence of any one of SEQ ID NOs: 1587-1627 or 1747.

F166. The molecule of any one of F159-F165, wherein the second polypeptide comprises the amino acid sequence of any one of SEQ ID NOs: 1587-1627.

F167. The molecule of any one of F159-F166, wherein the second polypeptide comprises the amino acid sequence of any one of SEQ ID NOs: 1587, 1592, 1596, 1615, or 1626.

F168. The molecule of any one of F159-F164, wherein the second polypeptide comprises the amino acid sequence of any one of SEQ ID NOs: 1587, 1592, 1596, 1615, 1626, 1818, 1822, 1825, or 1826.

F169. The molecule of any one of F159-F164, wherein the second polypeptide comprises the amino acid sequence of any one of SEQ ID NOs: 1587, 1592, 1615, 1626, 1818, 1822, 1825, or 1826.

F170. The molecule of any one of F159-F164, wherein the second polypeptide comprises the amino acid sequence of any one of SEQ ID NOs: 1626, 1818, 1822, 1825, or 1826.

F171. The molecule of any one of F159-F169, wherein the second polypeptide comprises the amino acid sequence of SEQ ID NO: 1587.

F172. The molecule of any one of F159-F169, wherein the second polypeptide comprises the amino acid sequence of SEQ ID NO: 1592.

F173. The molecule of any one of F159-F169, wherein the second polypeptide comprises the amino acid sequence of SEQ ID NO: 1596.

F174. The molecule of any one of F159-F169, wherein the second polypeptide comprises the amino acid sequence of SEQ ID NO: 1615.

F175. The molecule of any one of F159-F170, wherein the second polypeptide comprises the amino acid sequence of SEQ ID NO: 1626.

F176. The molecule of any one of F164 or F168-F170, wherein the second polypeptide comprises the amino acid sequence of SEQ ID NO: 1818.

F177. The molecule of any one of F164 or F168-F170, wherein the second polypeptide comprises the amino acid sequence of SEQ ID NO: 1822.

F178. The molecule of any one of F164 or F168-F170, wherein the second polypeptide comprises the amino acid sequence of SEQ ID NO: 1825.

F179. The molecule of any one of F164 or F168-F170, wherein the second polypeptide comprises the amino acid sequence of SEQ ID NO: 1826.

F180. The molecule of any one of F159-F179, wherein the first polypeptide has the same amino acid sequence as the second polypeptide.

F181. The molecule of any one of F159-F180, wherein the second linker polypeptide is a linear polypeptide.

F182. The molecule of any one of F159-F181, wherein the second linker polypeptide comprises the amino acid sequence of any one of SEQ ID NOs: 1628-1683, 1739-1746, or 1841-1852.

F183. The molecule of any one of F160-F182, wherein the second linker polypeptide comprises the amino acid sequence of any one of SEQ ID NOs: 1628-1683.

F184. The molecule of any one of F160-F183, wherein the second linker polypeptide comprises the amino acid sequence of any one of SEQ ID NOs: 1628-1630.

F185. The molecule of any one of F160-F184, wherein the second linker polypeptide comprises the amino acid sequence of SEQ ID NO: 1628.

F186. The molecule of any one of F160-F184, wherein the second linker polypeptide comprises the amino acid sequence of SEQ ID NO: 1629.

F187. The molecule of any one of F160-F184, wherein the second linker polypeptide comprises the amino acid sequence of SEQ ID NO: 1630.

F188. The molecule of any one of F160-F187, wherein the first linker polypeptide has the same amino acid sequence as the second linker polypeptide.

F189. The molecule of any one of F160-F188, wherein the cysteine residue of the antibody that is conjugated to the second linker polypeptide is at a position selected from the group consisting of 88 of a light chain, 384 of a heavy chain, and 487 of a heavy chain, according to AHo numbering.

F190. The molecule of F189, wherein the cysteine residue of the antibody that is conjugated to the second linker polypeptide is at position 384 of a heavy chain, according to AHo numbering.

F191. The molecule of any one of F160-F189, wherein the cysteine residues of the antibody that are conjugated to the first linker polypeptide and the second linker polypeptide are at positions selected from the group consisting of 88 of both light chains, 384 of both heavy chains, and 487 of both heavy chains, according to AHo numbering.

F192. The molecule of F191, wherein the cysteine residues of the antibody that are conjugated to the first linker polypeptide and the second linker polypeptide are at positions 384 of both heavy chains, according to AHo numbering.

F193. The molecule of any one of F119-F125 or F129-F192, wherein the antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise amino acid sequences selected from:
i. SEQ ID NO: 629, SEQ ID NO: 786, SEQ ID NO: 943, SEQ ID NO: 1100, SEQ ID NO: 1257, and SEQ ID NO: 1414, respectively;
ii. SEQ ID NO: 630, SEQ ID NO: 787, SEQ ID NO: 944, SEQ ID NO: 1101, SEQ ID NO: 1258, and SEQ ID NO: 1415, respectively;
iii. SEQ ID NO: 631, SEQ ID NO: 788, SEQ ID NO: 945, SEQ ID NO: 1102, SEQ ID NO: 1259, and SEQ ID NO: 1416, respectively;
iv. SEQ ID NO: 632, SEQ ID NO: 789, SEQ ID NO: 946, SEQ ID NO: 1103, SEQ ID NO: 1260, and SEQ ID NO: 1417, respectively;
v. SEQ ID NO: 633, SEQ ID NO: 790, SEQ ID NO: 947, SEQ ID NO: 1104, SEQ ID NO: 1261, and SEQ ID NO: 1418, respectively;
vi. SEQ ID NO: 634, SEQ ID NO: 791, SEQ ID NO: 948, SEQ ID NO: 1105, SEQ ID NO: 1262, and SEQ ID NO: 1419, respectively;
vii. SEQ ID NO: 635, SEQ ID NO: 792, SEQ ID NO: 949, SEQ ID NO: 1106, SEQ ID NO: 1263, and SEQ ID NO: 1420, respectively;
viii. SEQ ID NO: 636, SEQ ID NO: 793, SEQ ID NO: 950, SEQ ID NO: 1107, SEQ ID NO: 1264, and SEQ ID NO: 1421, respectively;
ix. SEQ ID NO: 637, SEQ ID NO: 794, SEQ ID NO: 951, SEQ ID NO: 1108, SEQ ID NO: 1265, and SEQ ID NO: 1422, respectively;
x. SEQ ID NO: 638, SEQ ID NO: 795, SEQ ID NO: 952, SEQ ID NO: 1109, SEQ ID NO: 1266, and SEQ ID NO: 1423, respectively;
xi. SEQ ID NO: 639, SEQ ID NO: 796, SEQ ID NO: 953, SEQ ID NO: 1110, SEQ ID NO: 1267, and SEQ ID NO: 1424, respectively;
xii. SEQ ID NO: 640, SEQ ID NO: 797, SEQ ID NO: 954, SEQ ID NO: 1111, SEQ ID NO: 1268, and SEQ ID NO: 1425, respectively;
xiii. SEQ ID NO: 641, SEQ ID NO: 798, SEQ ID NO: 955, SEQ ID NO: 1112, SEQ ID NO: 1269, and SEQ ID NO: 1426, respectively;
xiv. SEQ ID NO: 642, SEQ ID NO: 799, SEQ ID NO: 956, SEQ ID NO: 1113, SEQ ID NO: 1270, and SEQ ID NO: 1427, respectively;
xv. SEQ ID NO: 643, SEQ ID NO: 800, SEQ ID NO: 957, SEQ ID NO: 1114, SEQ ID NO: 1271, and SEQ ID NO: 1428, respectively;
xvi. SEQ ID NO: 644, SEQ ID NO: 801, SEQ ID NO: 958, SEQ ID NO: 1115, SEQ ID NO: 1272, and SEQ ID NO: 1429, respectively;
xvii. SEQ ID NO: 645, SEQ ID NO: 802, SEQ ID NO: 959, SEQ ID NO: 1116, SEQ ID NO: 1273, and SEQ ID NO: 1430, respectively;
xviii. SEQ ID NO: 646, SEQ ID NO: 803, SEQ ID NO: 960, SEQ ID NO: 1117, SEQ ID NO: 1274, and SEQ ID NO: 1431, respectively;
xix. SEQ ID NO: 647, SEQ ID NO: 804, SEQ ID NO: 961, SEQ ID NO: 1118, SEQ ID NO: 1275, and SEQ ID NO: 1432, respectively;
xx. SEQ ID NO: 648, SEQ ID NO: 805, SEQ ID NO: 962, SEQ ID NO: 1119, SEQ ID NO: 1276, and SEQ ID NO: 1433, respectively;
xxi. SEQ ID NO: 649, SEQ ID NO: 806, SEQ ID NO: 963, SEQ ID NO: 1120, SEQ ID NO: 1277, and SEQ ID NO: 1434, respectively;
xxii. SEQ ID NO: 650, SEQ ID NO: 807, SEQ ID NO: 964, SEQ ID NO: 1121, SEQ ID NO: 1278, and SEQ ID NO: 1435, respectively;
xxiii. SEQ ID NO: 651, SEQ ID NO: 808, SEQ ID NO: 965, SEQ ID NO: 1122, SEQ ID NO: 1279, and SEQ ID NO: 1436, respectively;
xxiv. SEQ ID NO: 652, SEQ ID NO: 809, SEQ ID NO: 966, SEQ ID NO: 1123, SEQ ID NO: 1280, and SEQ ID NO: 1437, respectively;
xxv. SEQ ID NO: 653, SEQ ID NO: 810, SEQ ID NO: 967, SEQ ID NO: 1124, SEQ ID NO: 1281, and SEQ ID NO: 1438, respectively;
xxvi. SEQ ID NO: 654, SEQ ID NO: 811, SEQ ID NO: 968, SEQ ID NO: 1125, SEQ ID NO: 1282, and SEQ ID NO: 1439, respectively;
xxvii. SEQ ID NO: 655, SEQ ID NO: 812, SEQ ID NO: 969, SEQ ID NO: 1126, SEQ ID NO: 1283, and SEQ ID NO: 1440, respectively;
xxviii. SEQ ID NO: 656, SEQ ID NO: 813, SEQ ID NO: 970, SEQ ID NO: 1127, SEQ ID NO: 1284, and SEQ ID NO: 1441, respectively;
xxix. SEQ ID NO: 657, SEQ ID NO: 814, SEQ ID NO: 971, SEQ ID NO: 1128, SEQ ID NO: 1285, and SEQ ID NO: 1442, respectively;
xxx. SEQ ID NO: 658, SEQ ID NO: 815, SEQ ID NO: 972, SEQ ID NO: 1129, SEQ ID NO: 1286, and SEQ ID NO: 1443, respectively;
xxxi. SEQ ID NO: 659, SEQ ID NO: 816, SEQ ID NO: 973, SEQ ID NO: 1130, SEQ ID NO: 1287, and SEQ ID NO: 1444, respectively;
xxxii. SEQ ID NO: 660, SEQ ID NO: 817, SEQ ID NO: 974, SEQ ID NO: 1131, SEQ ID NO: 1288, and SEQ ID NO: 1445, respectively;
xxxiii. SEQ ID NO: 661, SEQ ID NO: 818, SEQ ID NO: 975, SEQ ID NO: 1132, SEQ ID NO: 1289, and SEQ ID NO: 1446, respectively;
xxxiv. SEQ ID NO: 662, SEQ ID NO: 819, SEQ ID NO: 976, SEQ ID NO: 1133, SEQ ID NO: 1290, and SEQ ID NO: 1447, respectively;
xxxv. SEQ ID NO: 663, SEQ ID NO: 820, SEQ ID NO: 977, SEQ ID NO: 1134, SEQ ID NO: 1291, and SEQ ID NO: 1448, respectively;
xxxvi. SEQ ID NO: 664, SEQ ID NO: 821, SEQ ID NO: 978, SEQ ID NO: 1135, SEQ ID NO: 1292, and SEQ ID NO: 1449, respectively;
xxxvii. SEQ ID NO: 665, SEQ ID NO: 822, SEQ ID NO: 979, SEQ ID NO: 1136, SEQ ID NO: 1293, and SEQ ID NO: 1450, respectively;
xxxviii. SEQ ID NO: 666, SEQ ID NO: 823, SEQ ID NO: 980, SEQ ID NO: 1137, SEQ ID NO: 1294, and SEQ ID NO: 1451, respectively;
xxxix. SEQ ID NO: 667, SEQ ID NO: 824, SEQ ID NO: 981, SEQ ID NO: 1138, SEQ ID NO: 1295, and SEQ ID NO: 1452, respectively;
xl. SEQ ID NO: 668, SEQ ID NO: 825, SEQ ID NO: 982, SEQ ID NO: 1139, SEQ ID NO: 1296, and SEQ ID NO: 1453, respectively;
xli. SEQ ID NO: 669, SEQ ID NO: 826, SEQ ID NO: 983, SEQ ID NO: 1140, SEQ ID NO: 1297, and SEQ ID NO: 1454, respectively;
xlii. SEQ ID NO: 670, SEQ ID NO: 827, SEQ ID NO: 984, SEQ ID NO: 1141, SEQ ID NO: 1298, and SEQ ID NO: 1455, respectively;
xliii. SEQ ID NO: 671, SEQ ID NO: 828, SEQ ID NO: 985, SEQ ID NO: 1142, SEQ ID NO: 1299, and SEQ ID NO: 1456, respectively;
xliv. SEQ ID NO: 672, SEQ ID NO: 829, SEQ ID NO: 986, SEQ ID NO: 1143, SEQ ID NO: 1300, and SEQ ID NO: 1457, respectively;
xlv. SEQ ID NO: 673, SEQ ID NO: 830, SEQ ID NO: 987, SEQ ID NO: 1144, SEQ ID NO: 1301, and SEQ ID NO: 1458, respectively;
xlvi. SEQ ID NO: 674, SEQ ID NO: 831, SEQ ID NO: 988, SEQ ID NO: 1145, SEQ ID NO: 1302, and SEQ ID NO: 1459, respectively;
xlvii. SEQ ID NO: 675, SEQ ID NO: 832, SEQ ID NO: 989, SEQ ID NO: 1146, SEQ ID NO: 1303, and SEQ ID NO: 1460, respectively;
xlviii. SEQ ID NO: 676, SEQ ID NO: 833, SEQ ID NO: 990, SEQ ID NO: 1147, SEQ ID NO: 1304, and SEQ ID NO: 1461, respectively;
xlix. SEQ ID NO: 677, SEQ ID NO: 834, SEQ ID NO: 991, SEQ ID NO: 1148, SEQ ID NO: 1305, and SEQ ID NO: 1462, respectively;
l. SEQ ID NO: 678, SEQ ID NO: 835, SEQ ID NO: 992, SEQ ID NO: 1149, SEQ ID NO: 1306, and SEQ ID NO: 1463, respectively;
li. SEQ ID NO: 679, SEQ ID NO: 836, SEQ ID NO: 993, SEQ ID NO: 1150, SEQ ID NO: 1307, and SEQ ID NO: 1464, respectively;
lii. SEQ ID NO: 680, SEQ ID NO: 837, SEQ ID NO: 994, SEQ ID NO: 1151, SEQ ID NO: 1308, and SEQ ID NO: 1465, respectively;
liii. SEQ ID NO: 681, SEQ ID NO: 838, SEQ ID NO: 995, SEQ ID NO: 1152, SEQ ID NO: 1309, and SEQ ID NO: 1466, respectively;
liv. SEQ ID NO: 682, SEQ ID NO: 839, SEQ ID NO: 996, SEQ ID NO: 1153, SEQ ID NO: 1310, and SEQ ID NO: 1467, respectively;
lv. SEQ ID NO: 683, SEQ ID NO: 840, SEQ ID NO: 997, SEQ ID NO: 1154, SEQ ID NO: 1311, and SEQ ID NO: 1468, respectively;
lvi. SEQ ID NO: 684, SEQ ID NO: 841, SEQ ID NO: 998, SEQ ID NO: 1155, SEQ ID NO: 1312, and SEQ ID NO: 1469, respectively;
lvii. SEQ ID NO: 685, SEQ ID NO: 842, SEQ ID NO: 999, SEQ ID NO: 1156, SEQ ID NO: 1313, and SEQ ID NO: 1470, respectively;
lviii. SEQ ID NO: 686, SEQ ID NO: 843, SEQ ID NO: 1000, SEQ ID NO: 1157, SEQ ID NO: 1314, and SEQ ID NO: 1471, respectively;
lix. SEQ ID NO: 687, SEQ ID NO: 844, SEQ ID NO: 1001, SEQ ID NO: 1158, SEQ ID NO: 1315, and SEQ ID NO: 1472, respectively;
lx. SEQ ID NO: 688, SEQ ID NO: 845, SEQ ID NO: 1002, SEQ ID NO: 1159, SEQ ID NO: 1316, and SEQ ID NO: 1473, respectively;
lxi. SEQ ID NO: 689, SEQ ID NO: 846, SEQ ID NO: 1003, SEQ ID NO: 1160, SEQ ID NO: 1317, and SEQ ID NO: 1474, respectively;
lxii. SEQ ID NO: 690, SEQ ID NO: 847, SEQ ID NO: 1004, SEQ ID NO: 1161, SEQ ID NO: 1318, and SEQ ID NO: 1475, respectively;
lxiii. SEQ ID NO: 691, SEQ ID NO: 848, SEQ ID NO: 1005, SEQ ID NO: 1162, SEQ ID NO: 1319, and SEQ ID NO: 1476, respectively;
lxiv. SEQ ID NO: 692, SEQ ID NO: 849, SEQ ID NO: 1006, SEQ ID NO: 1163, SEQ ID NO: 1320, and SEQ ID NO: 1477, respectively;
lxv. SEQ ID NO: 693, SEQ ID NO: 850, SEQ ID NO: 1007, SEQ ID NO: 1164, SEQ ID NO: 1321, and SEQ ID NO: 1478, respectively;
lxvi. SEQ ID NO: 694, SEQ ID NO: 851, SEQ ID NO: 1008, SEQ ID NO: 1165, SEQ ID NO: 1322, and SEQ ID NO: 1479, respectively;
lxvii. SEQ ID NO: 695, SEQ ID NO: 852, SEQ ID NO: 1009, SEQ ID NO: 1166, SEQ ID NO: 1323, and SEQ ID NO: 1480, respectively;
lxviii. SEQ ID NO: 696, SEQ ID NO: 853, SEQ ID NO: 1010, SEQ ID NO: 1167, SEQ ID NO: 1324, and SEQ ID NO: 1481, respectively;
lxix. SEQ ID NO: 697, SEQ ID NO: 854, SEQ ID NO: 1011, SEQ ID NO: 1168, SEQ ID NO: 1325, and SEQ ID NO: 1482, respectively;
lxx. SEQ ID NO: 698, SEQ ID NO: 855, SEQ ID NO: 1012, SEQ ID NO: 1169, SEQ ID NO: 1326, and SEQ ID NO: 1483, respectively;
lxxi. SEQ ID NO: 699, SEQ ID NO: 856, SEQ ID NO: 1013, SEQ ID NO: 1170, SEQ ID NO: 1327, and SEQ ID NO: 1484, respectively;
lxxii. SEQ ID NO: 700, SEQ ID NO: 857, SEQ ID NO: 1014, SEQ ID NO: 1171, SEQ ID NO: 1328, and SEQ ID NO: 1485, respectively;
lxxiii. SEQ ID NO: 701, SEQ ID NO: 858, SEQ ID NO: 1015, SEQ ID NO: 1172, SEQ ID NO: 1329, and SEQ ID NO: 1486, respectively;
lxxiv. SEQ ID NO: 702, SEQ ID NO: 859, SEQ ID NO: 1016, SEQ ID NO: 1173, SEQ ID NO: 1330, and SEQ ID NO: 1487, respectively;
lxxv. SEQ ID NO: 703, SEQ ID NO: 860, SEQ ID NO: 1017, SEQ ID NO: 1174, SEQ ID NO: 1331, and SEQ ID NO: 1488, respectively;
lxxvi. SEQ ID NO: 704, SEQ ID NO: 861, SEQ ID NO: 1018, SEQ ID NO: 1175, SEQ ID NO: 1332, and SEQ ID NO: 1489, respectively;
lxxvii. SEQ ID NO: 705, SEQ ID NO: 862, SEQ ID NO: 1019, SEQ ID NO: 1176, SEQ ID NO: 1333, and SEQ ID NO: 1490, respectively;
lxxviii. SEQ ID NO: 706, SEQ ID NO: 863, SEQ ID NO: 1020, SEQ ID NO: 1177, SEQ ID NO: 1334, and SEQ ID NO: 1491, respectively;
lxxix. SEQ ID NO: 707, SEQ ID NO: 864, SEQ ID NO: 1021, SEQ ID NO: 1178, SEQ ID NO: 1335, and SEQ ID NO: 1492, respectively;
lxxx. SEQ ID NO: 708, SEQ ID NO: 865, SEQ ID NO: 1022, SEQ ID NO: 1179, SEQ ID NO: 1336, and SEQ ID NO: 1493, respectively;
lxxxi. SEQ ID NO: 709, SEQ ID NO: 866, SEQ ID NO: 1023, SEQ ID NO: 1180, SEQ ID NO: 1337, and SEQ ID NO: 1494, respectively;
lxxxii. SEQ ID NO: 710, SEQ ID NO: 867, SEQ ID NO: 1024, SEQ ID NO: 1181, SEQ ID NO: 1338, and SEQ ID NO: 1495, respectively;
lxxxiii. SEQ ID NO: 711, SEQ ID NO: 868, SEQ ID NO: 1025, SEQ ID NO: 1182, SEQ ID NO: 1339, and SEQ ID NO: 1496, respectively;
lxxxiv. SEQ ID NO: 712, SEQ ID NO: 869, SEQ ID NO: 1026, SEQ ID NO: 1183, SEQ ID NO: 1340, and SEQ ID NO: 1497, respectively;
lxxxv. SEQ ID NO: 713, SEQ ID NO: 870, SEQ ID NO: 1027, SEQ ID NO: 1184, SEQ ID NO: 1341, and SEQ ID NO: 1498, respectively;
lxxxvi. SEQ ID NO: 714, SEQ ID NO: 871, SEQ ID NO: 1028, SEQ ID NO: 1185, SEQ ID NO: 1342, and SEQ ID NO: 1499, respectively;
lxxxvii. SEQ ID NO: 715, SEQ ID NO: 872, SEQ ID NO: 1029, SEQ ID NO: 1186, SEQ ID NO: 1343, and SEQ ID NO: 1500, respectively;
lxxxviii. SEQ ID NO: 716, SEQ ID NO: 873, SEQ ID NO: 1030, SEQ ID NO: 1187, SEQ ID NO: 1344, and SEQ ID NO: 1501, respectively;
lxxxix. SEQ ID NO: 717, SEQ ID NO: 874, SEQ ID NO: 1031, SEQ ID NO: 1188, SEQ ID NO: 1345, and SEQ ID NO: 1502, respectively;
xc. SEQ ID NO: 718, SEQ ID NO: 875, SEQ ID NO: 1032, SEQ ID NO: 1189, SEQ ID NO: 1346, and SEQ ID NO: 1503, respectively;
xci. SEQ ID NO: 719, SEQ ID NO: 876, SEQ ID NO: 1033, SEQ ID NO: 1190, SEQ ID NO: 1347, and SEQ ID NO: 1504, respectively;
xcii. SEQ ID NO: 720, SEQ ID NO: 877, SEQ ID NO: 1034, SEQ ID NO: 1191, SEQ ID NO: 1348, and SEQ ID NO: 1505, respectively;
xciii. SEQ ID NO: 721, SEQ ID NO: 878, SEQ ID NO: 1035, SEQ ID NO: 1192, SEQ ID NO: 1349, and SEQ ID NO: 1506, respectively;
xciv. SEQ ID NO: 722, SEQ ID NO: 879, SEQ ID NO: 1036, SEQ ID NO: 1193, SEQ ID NO: 1350, and SEQ ID NO: 1507, respectively;
xcv. SEQ ID NO: 723, SEQ ID NO: 880, SEQ ID NO: 1037, SEQ ID NO: 1194, SEQ ID NO: 1351, and SEQ ID NO: 1508, respectively;
xcvi. SEQ ID NO: 724, SEQ ID NO: 881, SEQ ID NO: 1038, SEQ ID NO: 1195, SEQ ID NO: 1352, and SEQ ID NO: 1509, respectively;
xcvii. SEQ ID NO: 725, SEQ ID NO: 882, SEQ ID NO: 1039, SEQ ID NO: 1196, SEQ ID NO: 1353, and SEQ ID NO: 1510, respectively;
xcviii. SEQ ID NO: 726, SEQ ID NO: 883, SEQ ID NO: 1040, SEQ ID NO: 1197, SEQ ID NO: 1354, and SEQ ID NO: 1511, respectively;
xcix. SEQ ID NO: 727, SEQ ID NO: 884, SEQ ID NO: 1041, SEQ ID NO: 1198, SEQ ID NO: 1355, and SEQ ID NO: 1512, respectively;
c. SEQ ID NO: 728, SEQ ID NO: 885, SEQ ID NO: 1042, SEQ ID NO: 1199, SEQ ID NO: 1356, and SEQ ID NO: 1513, respectively;
ci. SEQ ID NO: 729, SEQ ID NO: 886, SEQ ID NO: 1043, SEQ ID NO: 1200, SEQ ID NO: 1357, and SEQ ID NO: 1514, respectively;
cii. SEQ ID NO: 730, SEQ ID NO: 887, SEQ ID NO: 1044, SEQ ID NO: 1201, SEQ ID NO: 1358, and SEQ ID NO: 1515, respectively;
ciii. SEQ ID NO: 731, SEQ ID NO: 888, SEQ ID NO: 1045, SEQ ID NO: 1202, SEQ ID NO: 1359, and SEQ ID NO: 1516, respectively;
civ. SEQ ID NO: 732, SEQ ID NO: 889, SEQ ID NO: 1046, SEQ ID NO: 1203, SEQ ID NO: 1360, and SEQ ID NO: 1517, respectively;
cv. SEQ ID NO: 733, SEQ ID NO: 890, SEQ ID NO: 1047, SEQ ID NO: 1204, SEQ ID NO: 1361, and SEQ ID NO: 1518, respectively;
cvi. SEQ ID NO: 734, SEQ ID NO: 891, SEQ ID NO: 1048, SEQ ID NO: 1205, SEQ ID NO: 1362, and SEQ ID NO: 1519, respectively;
cvii. SEQ ID NO: 735, SEQ ID NO: 892, SEQ ID NO: 1049, SEQ ID NO: 1206, SEQ ID NO: 1363, and SEQ ID NO: 1520, respectively;
cviii. SEQ ID NO: 736, SEQ ID NO: 893, SEQ ID NO: 1050, SEQ ID NO: 1207, SEQ ID NO: 1364, and SEQ ID NO: 1521, respectively;
cix. SEQ ID NO: 737, SEQ ID NO: 894, SEQ ID NO: 1051, SEQ ID NO: 1208, SEQ ID NO: 1365, and SEQ ID NO: 1522, respectively;
cx. SEQ ID NO: 738, SEQ ID NO: 895, SEQ ID NO: 1052, SEQ ID NO: 1209, SEQ ID NO: 1366, and SEQ ID NO: 1523, respectively;
cxi. SEQ ID NO: 739, SEQ ID NO: 896, SEQ ID NO: 1053, SEQ ID NO: 1210, SEQ ID NO: 1367, and SEQ ID NO: 1524, respectively;
cxii. SEQ ID NO: 740, SEQ ID NO: 897, SEQ ID NO: 1054, SEQ ID NO: 1211, SEQ ID NO: 1368, and SEQ ID NO: 1525, respectively;
cxiii. SEQ ID NO: 741, SEQ ID NO: 898, SEQ ID NO: 1055, SEQ ID NO: 1212, SEQ ID NO: 1369, and SEQ ID NO: 1526, respectively;
cxiv. SEQ ID NO: 742, SEQ ID NO: 899, SEQ ID NO: 1056, SEQ ID NO: 1213, SEQ ID NO: 1370, and SEQ ID NO: 1527, respectively;
cxv. SEQ ID NO: 743, SEQ ID NO: 900, SEQ ID NO: 1057, SEQ ID NO: 1214, SEQ ID NO: 1371, and SEQ ID NO: 1528, respectively;
cxvi. SEQ ID NO: 744, SEQ ID NO: 901, SEQ ID NO: 1058, SEQ ID NO: 1215, SEQ ID NO: 1372, and SEQ ID NO: 1529, respectively;
cxvii. SEQ ID NO: 745, SEQ ID NO: 902, SEQ ID NO: 1059, SEQ ID NO: 1216, SEQ ID NO: 1373, and SEQ ID NO: 1530, respectively;
cxviii. SEQ ID NO: 746, SEQ ID NO: 903, SEQ ID NO: 1060, SEQ ID NO: 1217, SEQ ID NO: 1374, and SEQ ID NO: 1531, respectively;
cxix. SEQ ID NO: 747, SEQ ID NO: 904, SEQ ID NO: 1061, SEQ ID NO: 1218, SEQ ID NO: 1375, and SEQ ID NO: 1532, respectively;
cxx. SEQ ID NO: 748, SEQ ID NO: 905, SEQ ID NO: 1062, SEQ ID NO: 1219, SEQ ID NO: 1376, and SEQ ID NO: 1533, respectively;
cxxi. SEQ ID NO: 749, SEQ ID NO: 906, SEQ ID NO: 1063, SEQ ID NO: 1220, SEQ ID NO: 1377, and SEQ ID NO: 1534, respectively;
cxxii. SEQ ID NO: 750, SEQ ID NO: 907, SEQ ID NO: 1064, SEQ ID NO: 1221, SEQ ID NO: 1378, and SEQ ID NO: 1535, respectively;
cxxiii. SEQ ID NO: 751, SEQ ID NO: 908, SEQ ID NO: 1065, SEQ ID NO: 1222, SEQ ID NO: 1379, and SEQ ID NO: 1536, respectively;
cxxiv. SEQ ID NO: 752, SEQ ID NO: 909, SEQ ID NO: 1066, SEQ ID NO: 1223, SEQ ID NO: 1380, and SEQ ID NO: 1537, respectively;
cxxv. SEQ ID NO: 753, SEQ ID NO: 910, SEQ ID NO: 1067, SEQ ID NO: 1224, SEQ ID NO: 1381, and SEQ ID NO: 1538, respectively;
cxxvi. SEQ ID NO: 754, SEQ ID NO: 911, SEQ ID NO: 1068, SEQ ID NO: 1225, SEQ ID NO: 1382, and SEQ ID NO: 1539, respectively;
cxxvii. SEQ ID NO: 755, SEQ ID NO: 912, SEQ ID NO: 1069, SEQ ID NO: 1226, SEQ ID NO: 1383, and SEQ ID NO: 1540, respectively;
cxxviii. SEQ ID NO: 756, SEQ ID NO: 913, SEQ ID NO: 1070, SEQ ID NO: 1227, SEQ ID NO: 1384, and SEQ ID NO: 1541, respectively;
cxxix. SEQ ID NO: 757, SEQ ID NO: 914, SEQ ID NO: 1071, SEQ ID NO: 1228, SEQ ID NO: 1385, and SEQ ID NO: 1542, respectively;
cxxx. SEQ ID NO: 758, SEQ ID NO: 915, SEQ ID NO: 1072, SEQ ID NO: 1229, SEQ ID NO: 1386, and SEQ ID NO: 1543, respectively;
cxxxi. SEQ ID NO: 759, SEQ ID NO: 916, SEQ ID NO: 1073, SEQ ID NO: 1230, SEQ ID NO: 1387, and SEQ ID NO: 1544, respectively;
cxxxii. SEQ ID NO: 760, SEQ ID NO: 917, SEQ ID NO: 1074, SEQ ID NO: 1231, SEQ ID NO: 1388, and SEQ ID NO: 1545, respectively;
cxxxiii. SEQ ID NO: 761, SEQ ID NO: 918, SEQ ID NO: 1075, SEQ ID NO: 1232, SEQ ID NO: 1389, and SEQ ID NO: 1546, respectively;
cxxxiv. SEQ ID NO: 762, SEQ ID NO: 919, SEQ ID NO: 1076, SEQ ID NO: 1233, SEQ ID NO: 1390, and SEQ ID NO: 1547, respectively;
cxxxv. SEQ ID NO: 763, SEQ ID NO: 920, SEQ ID NO: 1077, SEQ ID NO: 1234, SEQ ID NO: 1391, and SEQ ID NO: 1548, respectively;
cxxxvi. SEQ ID NO: 764, SEQ ID NO: 921, SEQ ID NO: 1078, SEQ ID NO: 1235, SEQ ID NO: 1392, and SEQ ID NO: 1549, respectively;
cxxxvii. SEQ ID NO: 765, SEQ ID NO: 922, SEQ ID NO: 1079, SEQ ID NO: 1236, SEQ ID NO: 1393, and SEQ ID NO: 1550, respectively;
cxxxviii. SEQ ID NO: 766, SEQ ID NO: 923, SEQ ID NO: 1080, SEQ ID NO: 1237, SEQ ID NO: 1394, and SEQ ID NO: 1551, respectively;
cxxxix. SEQ ID NO: 767, SEQ ID NO: 924, SEQ ID NO: 1081, SEQ ID NO: 1238, SEQ ID NO: 1395, and SEQ ID NO: 1552, respectively;
cxl. SEQ ID NO: 768, SEQ ID NO: 925, SEQ ID NO: 1082, SEQ ID NO: 1239, SEQ ID NO: 1396, and SEQ ID NO: 1553, respectively;
cxli. SEQ ID NO: 769, SEQ ID NO: 926, SEQ ID NO: 1083, SEQ ID NO: 1240, SEQ ID NO: 1397, and SEQ ID NO: 1554, respectively;
cxlii. SEQ ID NO: 770, SEQ ID NO: 927, SEQ ID NO: 1084, SEQ ID NO: 1241, SEQ ID NO: 1398, and SEQ ID NO: 1555, respectively;
cxliii. SEQ ID NO: 771, SEQ ID NO: 928, SEQ ID NO: 1085, SEQ ID NO: 1242, SEQ ID NO: 1399, and SEQ ID NO: 1556, respectively;
cxliv. SEQ ID NO: 772, SEQ ID NO: 929, SEQ ID NO: 1086, SEQ ID NO: 1243, SEQ ID NO: 1400, and SEQ ID NO: 1557, respectively;
cxlv. SEQ ID NO: 773, SEQ ID NO: 930, SEQ ID NO: 1087, SEQ ID NO: 1244, SEQ ID NO: 1401, and SEQ ID NO: 1558, respectively;
cxlvi. SEQ ID NO: 774, SEQ ID NO: 931, SEQ ID NO: 1088, SEQ ID NO: 1245, SEQ ID NO: 1402, and SEQ ID NO: 1559, respectively;
cxlvii. SEQ ID NO: 775, SEQ ID NO: 932, SEQ ID NO: 1089, SEQ ID NO: 1246, SEQ ID NO: 1403, and SEQ ID NO: 1560, respectively;
cxlviii. SEQ ID NO: 776, SEQ ID NO: 933, SEQ ID NO: 1090, SEQ ID NO: 1247, SEQ ID NO: 1404, and SEQ ID NO: 1561, respectively;
cxlix. SEQ ID NO: 777, SEQ ID NO: 934, SEQ ID NO: 1091, SEQ ID NO: 1248, SEQ ID NO: 1405, and SEQ ID NO: 1562, respectively;
cl. SEQ ID NO: 778, SEQ ID NO: 935, SEQ ID NO: 1092, SEQ ID NO: 1249, SEQ ID NO: 1406, and SEQ ID NO: 1563, respectively;
cli. SEQ ID NO: 779, SEQ ID NO: 936, SEQ ID NO: 1093, SEQ ID NO: 1250, SEQ ID NO: 1407, and SEQ ID NO: 1564, respectively;
clii. SEQ ID NO: 780, SEQ ID NO: 937, SEQ ID NO: 1094, SEQ ID NO: 1251, SEQ ID NO: 1408, and SEQ ID NO: 1565, respectively;
cliii. SEQ ID NO: 781, SEQ ID NO: 938, SEQ ID NO: 1095, SEQ ID NO: 1252, SEQ ID NO: 1409, and SEQ ID NO: 1566, respectively;
cliv. SEQ ID NO: 782, SEQ ID NO: 939, SEQ ID NO: 1096, SEQ ID NO: 1253, SEQ ID NO: 1410, and SEQ ID NO: 1567, respectively;
clv. SEQ ID NO: 783, SEQ ID NO: 940, SEQ ID NO: 1097, SEQ ID NO: 1254, SEQ ID NO: 1411, and SEQ ID NO: 1568, respectively;
clvi. SEQ ID NO: 784, SEQ ID NO: 941, SEQ ID NO: 1098, SEQ ID NO: 1255, SEQ ID NO: 1412, and SEQ ID NO: 1569, respectively; and
clvii. SEQ ID NO: 785, SEQ ID NO: 942, SEQ ID NO: 1099, SEQ ID NO: 1256, SEQ ID NO: 1413, and SEQ ID NO: 1570, respectively, preferably wherein the CDRL1, the CDRL2, the CDRL3, the CDRH1, the CDRH2, and the CDRH3 comprise the amino acid sequences of SEQ ID NO: 702, SEQ ID NO: 859, SEQ ID NO: 1016, SEQ ID NO: 1173, SEQ ID NO: 1330, and SEQ ID NO: 1487, respectively.

F194. The molecule of any one of F119-F125 or F129-F193, wherein the antibody comprises a light chain variable region and a heavy chain variable region, wherein the light chain variable region and the heavy chain variable region comprise amino acid sequences selected from:
i. SEQ ID NO: 1 and SEQ ID NO: 158, respectively;
ii. SEQ ID NO: 2 and SEQ ID NO: 159, respectively;
iii. SEQ ID NO: 3 and SEQ ID NO: 160, respectively;
iv. SEQ ID NO: 4 and SEQ ID NO: 161, respectively;
v. SEQ ID NO: 5 and SEQ ID NO: 162, respectively;
vi. SEQ ID NO: 6 and SEQ ID NO: 163, respectively;
vii. SEQ ID NO: 7 and SEQ ID NO: 164, respectively;
viii. SEQ ID NO: 8 and SEQ ID NO: 165, respectively;
ix. SEQ ID NO: 9 and SEQ ID NO: 166, respectively;
x. SEQ ID NO: 10 and SEQ ID NO: 167, respectively;
xi. SEQ ID NO: 11 and SEQ ID NO: 168, respectively;
xii. SEQ ID NO: 12 and SEQ ID NO: 169, respectively;
xiii. SEQ ID NO: 13 and SEQ ID NO: 170, respectively;
xiv. SEQ ID NO: 14 and SEQ ID NO: 171, respectively;
xv. SEQ ID NO: 15 and SEQ ID NO: 172, respectively;
xvi. SEQ ID NO: 16 and SEQ ID NO: 173, respectively;
xvii. SEQ ID NO: 17 and SEQ ID NO: 174, respectively;
xviii. SEQ ID NO: 18 and SEQ ID NO: 175, respectively;
xix. SEQ ID NO: 19 and SEQ ID NO: 176, respectively;
xx. SEQ ID NO: 20 and SEQ ID NO: 177, respectively;
xxi. SEQ ID NO: 21 and SEQ ID NO: 178, respectively;
xxii. SEQ ID NO: 22 and SEQ ID NO: 179, respectively;
xxiii. SEQ ID NO: 23 and SEQ ID NO: 180, respectively;
xxiv. SEQ ID NO: 24 and SEQ ID NO: 181, respectively;
xxv. SEQ ID NO: 25 and SEQ ID NO: 182, respectively;
xxvi. SEQ ID NO: 26 and SEQ ID NO: 183, respectively;
xxvii. SEQ ID NO: 27 and SEQ ID NO: 184, respectively;
xxviii. SEQ ID NO: 28 and SEQ ID NO: 185, respectively;
xxix. SEQ ID NO: 29 and SEQ ID NO: 186, respectively;
xxx. SEQ ID NO: 30 and SEQ ID NO: 187, respectively;
xxxi. SEQ ID NO: 31 and SEQ ID NO: 188, respectively;
xxxii. SEQ ID NO: 32 and SEQ ID NO: 189, respectively;
xxxiii. SEQ ID NO: 33 and SEQ ID NO: 190, respectively;
xxxiv. SEQ ID NO: 34 and SEQ ID NO: 191, respectively;
xxxv. SEQ ID NO: 35 and SEQ ID NO: 192, respectively;
xxxvi. SEQ ID NO: 36 and SEQ ID NO: 193, respectively;
xxxvii. SEQ ID NO: 37 and SEQ ID NO: 194, respectively;
xxxviii. SEQ ID NO: 38 and SEQ ID NO: 195, respectively;
xxxix. SEQ ID NO: 39 and SEQ ID NO: 196, respectively;
xl. SEQ ID NO: 40 and SEQ ID NO: 197, respectively;
xli. SEQ ID NO: 41 and SEQ ID NO: 198, respectively;
xlii. SEQ ID NO: 42 and SEQ ID NO: 199, respectively;
xliii. SEQ ID NO: 43 and SEQ ID NO: 200, respectively;
xliv. SEQ ID NO: 44 and SEQ ID NO: 201, respectively;
xlv. SEQ ID NO: 45 and SEQ ID NO: 202, respectively;
xlvi. SEQ ID NO: 46 and SEQ ID NO: 203, respectively;
xlvii. SEQ ID NO: 47 and SEQ ID NO: 204, respectively;
xlviii. SEQ ID NO: 48 and SEQ ID NO: 205, respectively;
xlix. SEQ ID NO: 49 and SEQ ID NO: 206, respectively;
l. SEQ ID NO: 50 and SEQ ID NO: 207, respectively;
li. SEQ ID NO: 51 and SEQ ID NO: 208, respectively;
lii. SEQ ID NO: 52 and SEQ ID NO: 209, respectively;
liii. SEQ ID NO: 53 and SEQ ID NO: 210, respectively;
liv. SEQ ID NO: 54 and SEQ ID NO: 211, respectively;
lv. SEQ ID NO: 55 and SEQ ID NO: 212, respectively;
lvi. SEQ ID NO: 56 and SEQ ID NO: 213, respectively;
lvii. SEQ ID NO: 57 and SEQ ID NO: 214, respectively;
lviii. SEQ ID NO: 58 and SEQ ID NO: 215, respectively;
lix. SEQ ID NO: 59 and SEQ ID NO: 216, respectively;
lx. SEQ ID NO: 60 and SEQ ID NO: 217, respectively;
lxi. SEQ ID NO: 61 and SEQ ID NO: 218, respectively;
lxii. SEQ ID NO: 62 and SEQ ID NO: 219, respectively;
lxiii. SEQ ID NO: 63 and SEQ ID NO: 220, respectively;
lxiv. SEQ ID NO: 64 and SEQ ID NO: 221, respectively;
lxv. SEQ ID NO: 65 and SEQ ID NO: 222, respectively;
lxvi. SEQ ID NO: 66 and SEQ ID NO: 223, respectively;
lxvii. SEQ ID NO: 67 and SEQ ID NO: 224, respectively;
lxviii. SEQ ID NO: 68 and SEQ ID NO: 225, respectively;
lxix. SEQ ID NO: 69 and SEQ ID NO: 226, respectively;
lxx. SEQ ID NO: 70 and SEQ ID NO: 227, respectively;
lxxi. SEQ ID NO: 71 and SEQ ID NO: 228, respectively;
lxxii. SEQ ID NO: 72 and SEQ ID NO: 229, respectively;
lxxiii. SEQ ID NO: 73 and SEQ ID NO: 230, respectively;
lxxiv. SEQ ID NO: 74 and SEQ ID NO: 231, respectively;
lxxv. SEQ ID NO: 75 and SEQ ID NO: 232, respectively;
lxxvi. SEQ ID NO: 76 and SEQ ID NO: 233, respectively;
lxxvii. SEQ ID NO: 77 and SEQ ID NO: 234, respectively;
lxxviii. SEQ ID NO: 78 and SEQ ID NO: 235, respectively;
lxxix. SEQ ID NO: 79 and SEQ ID NO: 236, respectively;
lxxx. SEQ ID NO: 80 and SEQ ID NO: 237, respectively;
lxxxi. SEQ ID NO: 81 and SEQ ID NO: 238, respectively;
lxxxii. SEQ ID NO: 82 and SEQ ID NO: 239, respectively;
lxxxiii. SEQ ID NO: 83 and SEQ ID NO: 240, respectively;
lxxxiv. SEQ ID NO: 84 and SEQ ID NO: 241, respectively;
lxxxv. SEQ ID NO: 85 and SEQ ID NO: 242, respectively;
lxxxvi. SEQ ID NO: 86 and SEQ ID NO: 243, respectively;
lxxxvii. SEQ ID NO: 87 and SEQ ID NO: 244, respectively;
lxxxviii. SEQ ID NO: 88 and SEQ ID NO: 245, respectively;
lxxxix. SEQ ID NO: 89 and SEQ ID NO: 246, respectively;
xc. SEQ ID NO: 90 and SEQ ID NO: 247, respectively;
xci. SEQ ID NO: 91 and SEQ ID NO: 248, respectively;
xcii. SEQ ID NO: 92 and SEQ ID NO: 249, respectively;
xciii. SEQ ID NO: 93 and SEQ ID NO: 250, respectively;
xciv. SEQ ID NO: 94 and SEQ ID NO: 251, respectively;
xcv. SEQ ID NO: 95 and SEQ ID NO: 252, respectively;
xcvi. SEQ ID NO: 96 and SEQ ID NO: 253, respectively;
xcvii. SEQ ID NO: 97 and SEQ ID NO: 254, respectively;
xcviii. SEQ ID NO: 98 and SEQ ID NO: 255, respectively;
xcix. SEQ ID NO: 99 and SEQ ID NO: 256, respectively;
c. SEQ ID NO: 100 and SEQ ID NO: 257, respectively;
ci. SEQ ID NO: 101 and SEQ ID NO: 258, respectively;
cii. SEQ ID NO: 102 and SEQ ID NO: 259, respectively;
ciii. SEQ ID NO: 103 and SEQ ID NO: 260, respectively;
civ. SEQ ID NO: 104 and SEQ ID NO: 261, respectively;
cv. SEQ ID NO: 105 and SEQ ID NO: 262, respectively;
cvi. SEQ ID NO: 106 and SEQ ID NO: 263, respectively;
cvii. SEQ ID NO: 107 and SEQ ID NO: 264, respectively;
cviii. SEQ ID NO: 108 and SEQ ID NO: 265, respectively;
cix. SEQ ID NO: 109 and SEQ ID NO: 266, respectively;
cx. SEQ ID NO: 110 and SEQ ID NO: 267, respectively;
cxi. SEQ ID NO: 111 and SEQ ID NO: 268, respectively;
cxii. SEQ ID NO: 112 and SEQ ID NO: 269, respectively;
cxiii. SEQ ID NO: 113 and SEQ ID NO: 270, respectively;
cxiv. SEQ ID NO: 114 and SEQ ID NO: 271, respectively;
cxv. SEQ ID NO: 115 and SEQ ID NO: 272, respectively;
cxvi. SEQ ID NO: 116 and SEQ ID NO: 273, respectively;
cxvii. SEQ ID NO: 117 and SEQ ID NO: 274, respectively;
cxviii. SEQ ID NO: 118 and SEQ ID NO: 275, respectively;
cxix. SEQ ID NO: 119 and SEQ ID NO: 276, respectively;
cxx. SEQ ID NO: 120 and SEQ ID NO: 277, respectively;
cxxi. SEQ ID NO: 121 and SEQ ID NO: 278, respectively;
cxxii. SEQ ID NO: 122 and SEQ ID NO: 279, respectively;
cxxiii. SEQ ID NO: 123 and SEQ ID NO: 280, respectively;
cxxiv. SEQ ID NO: 124 and SEQ ID NO: 281, respectively;
cxxv. SEQ ID NO: 125 and SEQ ID NO: 282, respectively;
cxxvi. SEQ ID NO: 126 and SEQ ID NO: 283, respectively;
cxxvii. SEQ ID NO: 127 and SEQ ID NO: 284, respectively;
cxxviii. SEQ ID NO: 128 and SEQ ID NO: 285, respectively;
cxxix. SEQ ID NO: 129 and SEQ ID NO: 286, respectively;
cxxx. SEQ ID NO: 130 and SEQ ID NO: 287, respectively;
cxxxi. SEQ ID NO: 131 and SEQ ID NO: 288, respectively;
cxxxii. SEQ ID NO: 132 and SEQ ID NO: 289, respectively;
cxxxiii. SEQ ID NO: 133 and SEQ ID NO: 290, respectively;
cxxxiv. SEQ ID NO: 134 and SEQ ID NO: 291, respectively;
cxxxv. SEQ ID NO: 135 and SEQ ID NO: 292, respectively;
cxxxvi. SEQ ID NO: 136 and SEQ ID NO: 293, respectively;
cxxxvii. SEQ ID NO: 137 and SEQ ID NO: 294, respectively;
cxxxviii. SEQ ID NO: 138 and SEQ ID NO: 295, respectively;
cxxxix. SEQ ID NO: 139 and SEQ ID NO: 296, respectively;
cxl. SEQ ID NO: 140 and SEQ ID NO: 297, respectively;
cxli. SEQ ID NO: 141 and SEQ ID NO: 298, respectively;
cxlii. SEQ ID NO: 142 and SEQ ID NO: 299, respectively;
cxliii. SEQ ID NO: 143 and SEQ ID NO: 300, respectively;
cxliv. SEQ ID NO: 144 and SEQ ID NO: 301, respectively;
cxlv. SEQ ID NO: 145 and SEQ ID NO: 302, respectively;
cxlvi. SEQ ID NO: 146 and SEQ ID NO: 303, respectively;
cxlvii. SEQ ID NO: 147 and SEQ ID NO: 304, respectively;
cxlviii. SEQ ID NO: 148 and SEQ ID NO: 305, respectively;
cxlix. SEQ ID NO: 149 and SEQ ID NO: 306, respectively;
cl. SEQ ID NO: 150 and SEQ ID NO: 307, respectively;
cli. SEQ ID NO: 151 and SEQ ID NO: 308, respectively;
clii. SEQ ID NO: 152 and SEQ ID NO: 309, respectively;
cliii. SEQ ID NO: 153 and SEQ ID NO: 310, respectively;
cliv. SEQ ID NO: 154 and SEQ ID NO: 311, respectively;
clv. SEQ ID NO: 155 and SEQ ID NO: 312, respectively;
clvi. SEQ ID NO: 156 and SEQ ID NO: 313, respectively; and
clvii. SEQ ID NO: 157 and SEQ ID NO: 314, respectively,
preferably wherein the light chain variable region and the heavy chain variable region comprise the amino acid sequences of SEQ ID NO: 74 and SEQ ID NO: 231, respectively.

F195. The molecule of any one of F119-F125 or F129-F194, wherein the antibody comprises a light chain and a heavy chain, wherein the light chain and heavy chain comprise amino acid sequences selected from:
i. SEQ ID NO: 315 and SEQ ID NO: 472, respectively;
ii. SEQ ID NO: 316 and SEQ ID NO: 473, respectively;
iii. SEQ ID NO: 317 and SEQ ID NO: 474, respectively;
iv. SEQ ID NO: 318 and SEQ ID NO: 475, respectively;
v. SEQ ID NO: 319 and SEQ ID NO: 476, respectively;
vi. SEQ ID NO: 320 and SEQ ID NO: 477, respectively;
vii. SEQ ID NO: 321 and SEQ ID NO: 478, respectively;
viii. SEQ ID NO: 322 and SEQ ID NO: 479, respectively;
ix. SEQ ID NO: 323 and SEQ ID NO: 480, respectively;
x. SEQ ID NO: 324 and SEQ ID NO: 481, respectively;
xi. SEQ ID NO: 325 and SEQ ID NO: 482, respectively;
xii. SEQ ID NO: 326 and SEQ ID NO: 483, respectively;
xiii. SEQ ID NO: 327 and SEQ ID NO: 484, respectively;
xiv. SEQ ID NO: 328 and SEQ ID NO: 485, respectively;
xv. SEQ ID NO: 329 and SEQ ID NO: 486, respectively;
xvi. SEQ ID NO: 330 and SEQ ID NO: 487, respectively;
xvii. SEQ ID NO: 331 and SEQ ID NO: 488, respectively;
xviii. SEQ ID NO: 332 and SEQ ID NO: 489, respectively;
xix. SEQ ID NO: 333 and SEQ ID NO: 490, respectively;
xx. SEQ ID NO: 334 and SEQ ID NO: 491, respectively;
xxi. SEQ ID NO: 335 and SEQ ID NO: 492, respectively;
xxii. SEQ ID NO: 336 and SEQ ID NO: 493, respectively;
xxiii. SEQ ID NO: 337 and SEQ ID NO: 494, respectively;
xxiv. SEQ ID NO: 338 and SEQ ID NO: 495, respectively;
xxv. SEQ ID NO: 339 and SEQ ID NO: 496, respectively;
xxvi. SEQ ID NO: 340 and SEQ ID NO: 497, respectively;
xxvii. SEQ ID NO: 341 and SEQ ID NO: 498, respectively;
xxviii. SEQ ID NO: 342 and SEQ ID NO: 499, respectively;
xxix. SEQ ID NO: 343 and SEQ ID NO: 500, respectively;
xxx. SEQ ID NO: 344 and SEQ ID NO: 501, respectively;
xxxi. SEQ ID NO: 345 and SEQ ID NO: 502, respectively;
xxxii. SEQ ID NO: 346 and SEQ ID NO: 503, respectively;
xxxiii. SEQ ID NO: 347 and SEQ ID NO: 504, respectively;
xxxiv. SEQ ID NO: 348 and SEQ ID NO: 505, respectively;
xxxv. SEQ ID NO: 349 and SEQ ID NO: 506, respectively;
xxxvi. SEQ ID NO: 350 and SEQ ID NO: 507, respectively;
xxxvii. SEQ ID NO: 351 and SEQ ID NO: 508, respectively;
xxxviii. SEQ ID NO: 352 and SEQ ID NO: 509, respectively;
xxxix. SEQ ID NO: 353 and SEQ ID NO: 510, respectively;
xl. SEQ ID NO: 354 and SEQ ID NO: 511, respectively;
xli. SEQ ID NO: 355 and SEQ ID NO: 512, respectively;
xlii. SEQ ID NO: 356 and SEQ ID NO: 513, respectively;
xliii. SEQ ID NO: 357 and SEQ ID NO: 514, respectively;
xliv. SEQ ID NO: 358 and SEQ ID NO: 515, respectively;
xlv. SEQ ID NO: 359 and SEQ ID NO: 516, respectively;
xlvi. SEQ ID NO: 360 and SEQ ID NO: 517, respectively;
xlvii. SEQ ID NO: 361 and SEQ ID NO: 518, respectively;
xlviii. SEQ ID NO: 362 and SEQ ID NO: 519, respectively;
xlix. SEQ ID NO: 363 and SEQ ID NO: 520, respectively;
l. SEQ ID NO: 364 and SEQ ID NO: 521, respectively;
li. SEQ ID NO: 365 and SEQ ID NO: 522, respectively;
lii. SEQ ID NO: 366 and SEQ ID NO: 523, respectively;
liii. SEQ ID NO: 367 and SEQ ID NO: 524, respectively;
liv. SEQ ID NO: 368 and SEQ ID NO: 525, respectively;
lv. SEQ ID NO: 369 and SEQ ID NO: 526, respectively;
lvi. SEQ ID NO: 370 and SEQ ID NO: 527, respectively;
lvii. SEQ ID NO: 371 and SEQ ID NO: 528, respectively;
lviii. SEQ ID NO: 372 and SEQ ID NO: 529, respectively;
lix. SEQ ID NO: 373 and SEQ ID NO: 530, respectively;
lx. SEQ ID NO: 374 and SEQ ID NO: 531, respectively;
lxi. SEQ ID NO: 375 and SEQ ID NO: 532, respectively;
lxii. SEQ ID NO: 376 and SEQ ID NO: 533, respectively;
lxiii. SEQ ID NO: 377 and SEQ ID NO: 534, respectively;
lxiv. SEQ ID NO: 378 and SEQ ID NO: 535, respectively;
lxv. SEQ ID NO: 379 and SEQ ID NO: 536, respectively;
lxvi. SEQ ID NO: 380 and SEQ ID NO: 537, respectively;
lxvii. SEQ ID NO: 381 and SEQ ID NO: 538, respectively;
lxviii. SEQ ID NO: 382 and SEQ ID NO: 539, respectively;
lxix. SEQ ID NO: 383 and SEQ ID NO: 540, respectively;
lxx. SEQ ID NO: 384 and SEQ ID NO: 541, respectively;
lxxi. SEQ ID NO: 385 and SEQ ID NO: 542, respectively;
lxxii. SEQ ID NO: 386 and SEQ ID NO: 543, respectively;
lxxiii. SEQ ID NO: 387 and SEQ ID NO: 544, respectively;
lxxiv. SEQ ID NO: 388 and SEQ ID NO: 545, respectively;
lxxv. SEQ ID NO: 389 and SEQ ID NO: 546, respectively;
lxxvi. SEQ ID NO: 390 and SEQ ID NO: 547, respectively;
lxxvii. SEQ ID NO: 391 and SEQ ID NO: 548, respectively;
lxxviii. SEQ ID NO: 392 and SEQ ID NO: 549, respectively;
lxxix. SEQ ID NO: 393 and SEQ ID NO: 550, respectively;
lxxx. SEQ ID NO: 394 and SEQ ID NO: 551, respectively;
lxxxi. SEQ ID NO: 395 and SEQ ID NO: 552, respectively;
lxxxii. SEQ ID NO: 396 and SEQ ID NO: 553, respectively;
lxxxiii. SEQ ID NO: 397 and SEQ ID NO: 554, respectively;
lxxxiv. SEQ ID NO: 398 and SEQ ID NO: 555, respectively;
lxxxv. SEQ ID NO: 399 and SEQ ID NO: 556, respectively;
lxxxvi. SEQ ID NO: 400 and SEQ ID NO: 557, respectively;
lxxxvii. SEQ ID NO: 401 and SEQ ID NO: 558, respectively;
lxxxviii. SEQ ID NO: 402 and SEQ ID NO: 559, respectively;
lxxxix. SEQ ID NO: 403 and SEQ ID NO: 560, respectively;
xc. SEQ ID NO: 404 and SEQ ID NO: 561, respectively;
xci. SEQ ID NO: 405 and SEQ ID NO: 562, respectively;
xcii. SEQ ID NO: 406 and SEQ ID NO: 563, respectively;
xciii. SEQ ID NO: 407 and SEQ ID NO: 564, respectively;
xciv. SEQ ID NO: 408 and SEQ ID NO: 565, respectively;
xcv. SEQ ID NO: 409 and SEQ ID NO: 566, respectively;
xcvi. SEQ ID NO: 410 and SEQ ID NO: 567, respectively;
xcvii. SEQ ID NO: 411 and SEQ ID NO: 568, respectively;
xcviii. SEQ ID NO: 412 and SEQ ID NO: 569, respectively;
xcix. SEQ ID NO: 413 and SEQ ID NO: 570, respectively;
c. SEQ ID NO: 414 and SEQ ID NO: 571, respectively;
ci. SEQ ID NO: 415 and SEQ ID NO: 572, respectively;
cii. SEQ ID NO: 416 and SEQ ID NO: 573, respectively;
ciii. SEQ ID NO: 417 and SEQ ID NO: 574, respectively;
civ. SEQ ID NO: 418 and SEQ ID NO: 575, respectively;
cv. SEQ ID NO: 419 and SEQ ID NO: 576, respectively;
cvi. SEQ ID NO: 420 and SEQ ID NO: 577, respectively;
cvii. SEQ ID NO: 421 and SEQ ID NO: 578, respectively;
cviii. SEQ ID NO: 422 and SEQ ID NO: 579, respectively;
cix. SEQ ID NO: 423 and SEQ ID NO: 580, respectively;
cx. SEQ ID NO: 424 and SEQ ID NO: 581, respectively;
cxi. SEQ ID NO: 425 and SEQ ID NO: 582, respectively;
cxii. SEQ ID NO: 426 and SEQ ID NO: 583, respectively;
cxiii. SEQ ID NO: 427 and SEQ ID NO: 584, respectively;
cxiv. SEQ ID NO: 428 and SEQ ID NO: 585, respectively;
cxv. SEQ ID NO: 429 and SEQ ID NO: 586, respectively;
cxvi. SEQ ID NO: 430 and SEQ ID NO: 587, respectively;
cxvii. SEQ ID NO: 431 and SEQ ID NO: 588, respectively;
cxviii. SEQ ID NO: 432 and SEQ ID NO: 589, respectively;
cxix. SEQ ID NO: 433 and SEQ ID NO: 590, respectively;
cxx. SEQ ID NO: 434 and SEQ ID NO: 591, respectively;
cxxi. SEQ ID NO: 435 and SEQ ID NO: 592, respectively;
cxxii. SEQ ID NO: 436 and SEQ ID NO: 593, respectively;
cxxiii. SEQ ID NO: 437 and SEQ ID NO: 594, respectively;
cxxiv. SEQ ID NO: 438 and SEQ ID NO: 595, respectively;
cxxv. SEQ ID NO: 439 and SEQ ID NO: 596, respectively;
cxxvi. SEQ ID NO: 440 and SEQ ID NO: 597, respectively;
cxxvii. SEQ ID NO: 441 and SEQ ID NO: 598, respectively;
cxxviii. SEQ ID NO: 442 and SEQ ID NO: 599, respectively;
cxxix. SEQ ID NO: 443 and SEQ ID NO: 600, respectively;
cxxx. SEQ ID NO: 444 and SEQ ID NO: 601, respectively;
cxxxi. SEQ ID NO: 445 and SEQ ID NO: 602, respectively;
cxxxii. SEQ ID NO: 446 and SEQ ID NO: 603, respectively;
cxxxiii. SEQ ID NO: 447 and SEQ ID NO: 604, respectively;
cxxxiv. SEQ ID NO: 448 and SEQ ID NO: 605, respectively;
cxxxv. SEQ ID NO: 449 and SEQ ID NO: 606, respectively;
cxxxvi. SEQ ID NO: 450 and SEQ ID NO: 607, respectively;
cxxxvii. SEQ ID NO: 451 and SEQ ID NO: 608, respectively;
cxxxviii. SEQ ID NO: 452 and SEQ ID NO: 609, respectively;
cxxxix. SEQ ID NO: 453 and SEQ ID NO: 610, respectively;
cxl. SEQ ID NO: 454 and SEQ ID NO: 611, respectively;
cxli. SEQ ID NO: 455 and SEQ ID NO: 612, respectively;
cxlii. SEQ ID NO: 456 and SEQ ID NO: 613, respectively;
cxliii. SEQ ID NO: 457 and SEQ ID NO: 614, respectively;
cxliv. SEQ ID NO: 458 and SEQ ID NO: 615, respectively;
cxlv. SEQ ID NO: 459 and SEQ ID NO: 616, respectively;
cxlvi. SEQ ID NO: 460 and SEQ ID NO: 617, respectively;
cxlvii. SEQ ID NO: 461 and SEQ ID NO: 618, respectively;
cxlviii. SEQ ID NO: 462 and SEQ ID NO: 619, respectively;
cxlix. SEQ ID NO: 463 and SEQ ID NO: 620, respectively;
cl. SEQ ID NO: 464 and SEQ ID NO: 621, respectively;
cli. SEQ ID NO: 465 and SEQ ID NO: 622, respectively;
clii. SEQ ID NO: 466 and SEQ ID NO: 623, respectively;
cliii. SEQ ID NO: 467 and SEQ ID NO: 624, respectively;
cliv. SEQ ID NO: 468 and SEQ ID NO: 625, respectively;
clv. SEQ ID NO: 469 and SEQ ID NO: 626, respectively;
clvi. SEQ ID NO: 470 and SEQ ID NO: 627, respectively; and
clvii. SEQ ID NO: 471 and SEQ ID NO: 628, respectively,
wherein the antibody comprises one or more cysteine amino acid substitution(s) at one or more position(s) selected from 88 of the light chain, 384 of the heavy chain, or 487 of the heavy chain, according to AHo numbering.

F196. The molecule of F195, wherein:
the light chain comprises the amino acid sequence of SEQ ID NO: 388 and the heavy chain comprises the amino acid sequence of SEQ ID NO: 1571; or
the light chain comprises the amino acid sequence of SEQ ID NO: 455 and the heavy chain comprises the amino acid sequence of SEQ ID NO: 1572; or
the light chain comprises the amino acid sequence of SEQ ID NO: 389 and the heavy chain comprises the amino acid sequence of SEQ ID NO: 1573; or
the light chain comprises the amino acid sequence of SEQ ID NO: 455 and the heavy chain comprises the amino acid sequence of SEQ ID NO: 1574; or
the light chain comprises the amino acid sequence of SEQ ID NO: 1575 and the heavy chain comprises the amino acid sequence of SEQ ID NO: 612,
preferably wherein the light chain comprises the amino acid sequence of SEQ ID NO: 388 and the heavy chain comprises the amino acid sequence of SEQ ID NO: 1571.

F197. A molecule of F195 or F196, wherein the light chain comprises the amino acid sequence of SEQ ID NO: 388 and the heavy chain comprises the amino acid sequence of SEQ ID NO: 1571.

F198. A molecule comprising:
a first polypeptide that agonizes a glucagon receptor (“GCGR”);
a first linker polypeptide;
an antibody that specifically binds to a glucose-dependent insulinotropic polypeptide receptor (“GIPR”);
a second linker polypeptide; and
a second polypeptide that agonizes a GCGR,
wherein:
   an ε-amino group of a lysine residue of the first polypeptide is covalently linked to a C-terminus of the first linker polypeptide;
   an N-terminus of the first linker polypeptide is conjugated to a cysteine residue of the antibody;
   an ε-amino group of a lysine residue of the second polypeptide is covalently linked to a C-terminus of the second linker polypeptide; and
   an N-terminus of the second linker polypeptide is conjugated to a cysteine residue of the antibody.

F199. A molecule comprising:
a first polypeptide that agonizes a glucagon receptor (“GCGR”);
a first linker polypeptide;
an antibody that specifically binds to a glucose-dependent insulinotropic polypeptide receptor (“GIPR”);
a second linker polypeptide; and
a second polypeptide that agonizes a GCGR,
wherein:
   a C-terminal amino acid residue of the first polypeptide is covalently linked to a N-terminal amino acid residue of the first linker polypeptide;
   a C-terminal amino acid residue of the first linker polypeptide is conjugated to a cysteine residue of the antibody;
   a C-terminal amino acid residue of the second polypeptide is covalently linked to a N-terminal amino acid residue of the second linker polypeptide; and
   a C-terminal amino acid of the second linker polypeptide is conjugated to a cysteine residue of the antibody.

F200. The molecule of F198 or F199, wherein each of the first linker polypeptide and the second linker polypeptide independently comprises the amino acid sequence of any one of SEQ ID NOs: 1628-1683, 1739-1746, or 1841-1852.

F201. The molecule of any one of F198-F200, wherein each of the first linker polypeptide and the second linker polypeptide independently comprises the amino acid sequence of any one of SEQ ID NOs: 1628-1683, preferably SEQ ID NOs: 1628-1630, preferably SEQ ID NO: 1628.

F202. The molecule of any one of F198-F201, wherein each of the first polypeptide and the second polypeptide independently comprises glucagon or a glucagon analog.

F203. The molecule of any one of F198-F202, wherein each of the first polypeptide and the second polypeptide is independently selected from the polypeptides of any one of F1-F55.

F204. The molecule of any one of F198-F203, wherein each of the first polypeptide and the second polypeptide independently comprises the amino acid sequence of any one of SEQ ID NOs: 1587-1627, 1747-1840, 1859-1862, or 1879-1881.

F205. The molecule of any one of F198-F204, wherein each of the first polypeptide and the second polypeptide independently comprises the amino acid sequence of any one of SEQ ID NOs: 1587-1627 or 1747.

F206. The molecule of any one of F198-F203, wherein each of the first polypeptide and the second polypeptide independently comprises the amino acid sequence of any one of SEQ ID NOs: 1626, 1818, 1822, 1825, or 1826.

F207. The molecule of F198-F205, wherein each of the first polypeptide and the second polypeptide independently comprises the amino acid sequence of any one of SEQ ID NOs: 1587, 1592, 1596, 1615, and 1626.

F208. The molecule of any one of F198-F207, wherein the first polypeptide has the same amino acid sequence as the second polypeptide.

F209. The molecule of any one of F198-F208, wherein the first linker polypeptide has the same amino acid sequence as the second linker polypeptide.

F210. The molecule of any one of F198-F209, wherein: the first polypeptide has the same amino acid sequence as the second polypeptide; and the first linker polypeptide has the same amino acid sequence as the second linker polypeptide.

F211. The molecule of any one of F198 or F200-F210, wherein the lysine residue of the first polypeptide comprising the ε-amino group that is covalently linked to the C-terminus of the first linker polypeptide is at position 24 or position 28.

F212. The molecule of any one of F198 or F200-F211, wherein the lysine residue of the second polypeptide comprising the ε-amino group that is covalently linked to the C-terminus of the second linker polypeptide is at position 24 or position 28.

F213. The molecule of any one of F198 or F200-F212, wherein: the lysine residue of the first polypeptide comprising the ε-amino group that is covalently linked to the C-terminus of the first linker polypeptide is at position 28; and the lysine residue of the second polypeptide comprising the ε-amino group that is covalently linked to the C-terminus of the second linker polypeptide is at position 28.

F214. The molecule of any one of F198-F213, wherein the cysteine residues of the antibody that are conjugated to the first linker polypeptide and the second linker polypeptide are at positions selected from the group consisting of 88 of both light chains, 384 of both heavy chains, and 487 of both heavy chains, according to AHo numbering.

F215. The molecule of any one of F198-F214, wherein the cysteine residues of the antibody that are conjugated to the first linker polypeptide and the second linker polypeptide are at positions 384 of both heavy chains, according to AHo numbering.

F216. The molecule of any one of F198 or F200-F215, wherein:
the lysine residue of the first polypeptide comprising the ε-amino group that is covalently linked to the C-terminus of the first linker polypeptide is at position 28;
the lysine residue of the second polypeptide comprising the ε-amino group that is covalently linked to the C-terminus of the second linker polypeptide is at position 28; and
the cysteine residues of the antibody that are conjugated to the N-termini of the first linker polypeptide and the second linker polypeptide are at positions 384 of both heavy chains, according to AHo numbering.

F217. The molecule of any one of F198-F216, wherein the antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise the amino acid sequences of SEQ ID NO: 702, SEQ ID NO: 859, SEQ ID NO: 1016, SEQ ID NO: 1173, SEQ ID NO: 1330, and SEQ ID NO: 1487, respectively.

F218. The molecule of any one of F198-F217, wherein the antibody comprises a light chain variable region comprising the amino acid sequence of SEQ ID NO: 74 and a heavy chain variable region comprising the amino acid sequence of SEQ ID NO: 231.

F219. The molecule of any one of F198-F218, wherein the antibody comprises a light chain comprising the amino acid sequence of SEQ ID NO: 388 and a heavy chain comprising the amino acid sequence of SEQ ID NO: 1571.

F220. A molecule comprising:
a polypeptide that agonizes a glucagon receptor (“GCGR”) comprising the amino acid sequence of SEQ ID NO: 1826;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

F221. A molecule comprising:
a polypeptide that agonizes a glucagon receptor (“GCGR”) comprising the amino acid sequence of SEQ ID NO: 1822;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 24 of the polypeptide is covalently linked to a C-terminus of the linker polypeptide; and
   an N-terminus of the linker polypeptide is conjugated to a cysteine residue at position 275 of the heavy chain of the antibody.

F222. A molecule comprising:
a polypeptide that agonizes a glucagon receptor (“GCGR”) comprising the amino acid sequence of SEQ ID NO: 1825;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

F223. A molecule comprising:
a polypeptide that agonizes a glucagon receptor (“GCGR”) comprising the amino acid sequence of SEQ ID NO: 1818;
a linker polypeptide comprising the amino acid sequence of SEQ ID NO: 1628; and
an antibody that specifically binds to a glucose-dependent insulinotropic polypeptide receptor (“GIPR”), wherein the antibody comprises a light chain and a heavy chain, wherein the light chain comprises the amino acid sequence of SEQ ID NO: 388 and the heavy chain comprises the amino acid sequence of SEQ ID NO: 1571,
wherein:
   an ε-amino group of a lysine residue at position 28 of the polypeptide is covalently linked to a C-terminus of the linker polypeptide; and
   an N-terminus of the linker polypeptide is conjugated to a cysteine residue at position 275 of the heavy chain of the antibody.

F224. A molecule comprising:
a first polypeptide and a second polypeptide that agonize a glucagon receptor (“GCGR”), wherein the first polypeptide and the second polypeptide each comprise the amino acid sequence of SEQ ID NO: 1822;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 24 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 24 of the second polypeptide is covalently linked to a C-terminus of the second linker polypeptide; and
   an N-terminus of the second linker polypeptide is conjugated to a cysteine residue at position 275 of the second heavy chain of the antibody.

F225. A molecule comprising:
a first polypeptide and a second polypeptide that agonize a glucagon receptor (“GCGR”), wherein the first polypeptide and the second polypeptide each comprise the amino acid sequence of SEQ ID NO: 1825;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

F225. A molecule comprising:
a first polypeptide and a second polypeptide that agonize a glucagon receptor (“GCGR”), wherein the first polypeptide and the second polypeptide each comprise the amino acid sequence of SEQ ID NO: 1826;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

F226. A molecule comprising:
a first polypeptide and a second polypeptide that agonize a glucagon receptor (“GCGR”), wherein the first polypeptide and the second polypeptide each comprise the amino acid sequence of SEQ ID NO: 1818;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
   an ε-amino group of a lysine residue at position 28 of the first polypeptide is covalently linked to a C-terminus of the first linker polypeptide;
   an N-terminus of the first linker polypeptide is conjugated to a cysteine residue at position 275 of the first heavy chain of the antibody;
   an ε-amino group of a lysine residue at position 28 of the second polypeptide is covalently linked to a C-terminus of the second linker polypeptide; and
   an N-terminus of the second linker polypeptide is conjugated to a cysteine residue at position 275 of the second heavy chain of the antibody.

F227. A pharmaceutical composition comprising:
a polypeptide of any one of F1-F55 or a molecule of any one of F56-F226; and a pharmaceutically acceptable excipient.

F228. A method of treating obesity in a subject in need thereof, the method comprising administering a polypeptide of any one of F1-F55, a molecule of any one of F56-F226, or a pharmaceutical composition of F227 to the subject.

F229. A polypeptide of any one of F1-F55, a molecule of any one of F56-F226, or a pharmaceutical composition of F227 for use in treating obesity.

F230. Use of a polypeptide of any one of F1-F55 or a molecule of any one of F56-F226 in the manufacture of a medicament for treating obesity.

F231. The method of F228, the polypeptide, molecule or pharmaceutical composition for use of F229, and the use of F230, wherein treating obesity comprises lowering blood glucose, insulin, triglyceride, or cholesterol levels; reducing body weight; and/or improving glucose tolerance, energy expenditure, or insulin sensitivity.

F232. A method of reducing body weight and/or food intake in a subject in need thereof, the method comprising administering a polypeptide of any one of F1-F55, a molecule of any one of F56-F226, or a pharmaceutical composition of F227 to the subject.

F233. A polypeptide of any one of F1-F55, a molecule of any one of F56-F226, or a pharmaceutical composition of F227 for use in reducing body weight and/or food intake in a subject in need thereof.

F234. Use of a polypeptide of any one of F1-F55 or a molecule of any one of F56-F226 in the manufacture of a medicament for reducing body weight and/or food intake in a subject in need thereof.

F235. The method of F232, the polypeptide, molecule or pharmaceutical composition for use of F233, and the use of F234, wherein the subject is overweight or obese.

F236. A method of treating obesity in a subject in need thereof, the method comprising administering a polypeptide of any one of F1-F55, a molecule of any one of F56-F226, or a pharmaceutical composition of F227 to the subject in combination with a GLP-1 agonist.

F237. A polypeptide of any one of F1-F55, a molecule of any one of F56-F226, or a pharmaceutical composition of F227 for use in a method of treating obesity in a subject in need thereof, wherein the method comprises administering the polypeptide, molecule, or pharmaceutical composition to the subject in combination with a GLP-1 agonist.

F238. The method of F236 or the polypeptide, molecule or pharmaceutical composition for use of F237, wherein treating obesity comprises lowering blood glucose, insulin, triglyceride, or cholesterol levels; reducing body weight; and/or improving glucose tolerance, energy expenditure, or insulin sensitivity.

F239. The method of F236 or the polypeptide, molecule or pharmaceutical composition for use of F237, wherein, prior to the administering, the subject experienced a weight-loss plateau.

F240. The method of F236 or the polypeptide, molecule or pharmaceutical composition for use of F237, wherein the GLP-1 agonist is semaglutide.

F241. A method of reducing body weight and/or food intake in a subject in need thereof, the method comprising administering a polypeptide of any one of F1-F55, a molecule of any one of F56-F226, or a pharmaceutical composition of F227 to the subject in combination with a GLP-1 agonist.

F242. A polypeptide of any one of F1-F55, a molecule of any one of F56-F226, or a pharmaceutical composition of F227 for use in a method of reducing body weight and/or food intake in a subject in need thereof, wherein the method comprises administering the polypeptide, molecule, or pharmaceutical composition to the subject in combination with a GLP-1 agonist.

F243. The method of F241 or the polypeptide, molecule, or pharmaceutical composition for use of F242, wherein the subject is overweight or obese.

F244. The method of F241 or the polypeptide, molecule, or pharmaceutical composition for use of F242, wherein the GLP-1 agonist is semaglutide.

F245. The method of F241 or the polypeptide, molecule, or pharmaceutical composition for use of F242, wherein the subject is overweight or obese and the GLP-1 agonist is semaglutide.

F246. The method of any one of F241 or F243-F245 or the polypeptide, molecule, or pharmaceutical composition for use of any one of F242-F245, wherein the GLP-1 agonist and the polypeptide, molecule, or pharmaceutical composition for use are administered in consecutive, non-overlapping dosing intervals, wherein the GLP-1 agonist is administered prior to the polypeptide, molecule, or pharmaceutical composition.

F247. The method of any one of F241 or F243-F245 or the polypeptide, molecule, or pharmaceutical composition for use of any one of F242-F245, wherein the GLP-1 agonist and the polypeptide, molecule, or pharmaceutical composition for use are administered concurrently.

F248. The method or the polypeptide, molecule, or pharmaceutical composition for use of F247, wherein at least one dose of the GLP-1 agonist is administered prior to a first administration of the polypeptide, molecule, or pharmaceutical composition.

The following examples are given for the purpose of illustrating various embodiments of the disclosure and are not meant to limit the present disclosure in any fashion. One skilled in the art will appreciate readily that the present disclosure is well-adapted to carry out the objects and obtain the ends and advantages mentioned, as well as those objects, ends, and advantages inherent herein. Changes therein and other uses which are encompassed within the spirit of the disclosure as defined by the scope of the claims will occur to those skilled in the art.

### EXAMPLES

This section provides specific examples of polypeptide agonists of human glucagon receptor, conjugates comprising the same, and methods of making and using the same.

### List of Abbreviations

° degree(s)
% percentage
Å angstrom(s)
ACN or MeCN or CH₃CN acetonitrile
aDNP anti-dinitrophenyl
aGIPR anti-glucose-dependent insulinotropic polypeptide receptor
ANOVA analysis of variance
BOC or Boc tert-butyloxycarbonyl
BSA bovine serum albumin
cAMP cyclic adenosine monophosphate
CHO Chinese hamster ovary
CLND chemiluminescent nitrogen detection
CV column volume(s)
Da dalton(s)
DCM dichloromethane
DHAA dehydroascorbic acid
DIC N,N-diisopropylcarbodiimide
DIO diet-induced obese
DMF N,N-dimethylformamide
DMSO dimethyl sulfoxide
DNP dinitrophenyl
DNP×GCG Conjugate comprising an anti-dinitrophenyl antibody and a glucagon receptor agonist
ECD extracellular domain
EDTA ethylenediaminetetraacetic acid
ELISA enzyme-linked immunosorbent assay
EPI epididymal
eq or eq. or equiv. equivalent
Fmoc fluorenylmethoxycarbonyl
g or gr gram(s)
GCG glucagon
GCGR glucagon receptor
GLP-1 glucagon-like peptide-1
GLP-1R glucagon-like peptide-1 receptor
GIP glucose-dependent insulinotropic polypeptide
GIPR glucose-dependent insulinotropic polypeptide receptor
GIPR×GCG Conjugate comprising an anti-glucose-dependent insulinotropic polypeptide receptor antibody and a glucagon receptor agonist
h or hr hour(s)
H₂O water
HCl hydrogen chloride
HEPES 4-(2-hydroxyethyl)-1-piperazineethanesulfonic acid
HPLC high pressure liquid chromatography
HTRF homogeneous time-resolved fluorescence
hu human
IBMX 3-isobutyl-1-methylxanthine
ING inguinal
i.p. intraperitoneal
IR infrared
ivDde (4,4-dimethyl-2,6-dioxocyclohex-1-ylidene)-3-methylbutyl
kDa kilodalton(s)
kg kilogram(s)
L liter(s)
LC MS, LCMS, LC-MS or LC/MS liquid chromatography mass spectroscopy
M molar
MBHA methylbenzhydryl
mg milligram(s)
min minute(s)
mL milliliter(s)
mM millimolar(s)
mpk milligram(s) (mg) per kilogram (kg)
Na₂CO₃ sodium carbonate
NaOAc sodium acetate
(NH₄)₂SO₄ ammonium sulfate
No. number
OGTT oral glucose tolerance test
Oxyma ethyl cyanohydroxyiminoacetate
P20 or PS20 polysorbate 20
PBS phosphate buffered saline
PES polyethersulfone
RP reverse phase
RT room temperature
SE or SEM standard error of the mean
SEC size exclusion chromatography
TFA trifluoroacetic acid
TGA triacylglycerol
TIS triisopropyl silane
TOF time-of-flight
TPPTS triphenylphosphine-3,3′,3″-trisulfonic acid trisodium salt
μg microgram
μM micromolar
WAT white adipose tissue

### Section 1: Synthesis of Antibody-Peptide Conjugates

Provided in this section is a description of a general procedure used to prepare the specific example antibody-peptide conjugates and Fc-peptide conjugates provided herein in Table 19. In Table 19, a conjugate number may be formatted as a five-digit number followed by a dash and a one-digit number. The five-digit number corresponds to the sequence of the conjugate, and, where present, the one-digit number refers to a lot number. Additionally, conjugate descriptions in Table 19 are written such that the antibody or Fc name precedes the dash and the numerical peptide-linker molecule identifier follows the dash. Additionally, in Table 19, the peptide attachment point describes the amino acid of the GCGR agonist peptide that connects to the linker polypeptide. The HC attachment point describes the amino acid of the anti-GIPR antibody that is connected to the linker polypeptide.

The GCGR agonist peptides in conjugates 40749, 40752, 40754, 43479, 43480, 91660, and 91661 are amidated at their C-termini.

### Part 1: Peptide-Linker Molecule Synthesis

Peptides with linkers were prepared by fluorenylmethoxycarbonyl (Fmoc)-based solid peptide synthesis using either 4-(2′,4′-dimethoxyphenyl-Fmoc-aminmethyl)-phenoxyacetamido-methylbenzhydryl amine resin (Rink Amide-MBHA Resin, ChemPep Inc.) to generate C-terminal amides or pre-substituted p-benzyloxybenzyl alcohol resin (pre-substituted Wang resin, Polymer Laboratories) to generate C-terminal acids. The syntheses were typically performed on either a MultiPep RSi (Intavis Inc.) at ambient temperature or a Gyros-Protein Technologies Tribute IR-Heated Synthesizer (50-75° C.) or a CEM Liberty Blue microwave-heated synthesizer (50-90° C.). The synthesizers utilized 20% v/v 4-methyl piperidine in N,N-dimethylformamide (DMF) for Fmoc removal and 1,3-diisopropylcarbodiimide (DIC)/ethyl cyano(hydroxyimino)acetate (oxyma) for amino acid coupling. Each residue was coupled with an excess of coupling solution (5.0 eq), and each coupling reaction was performed twice at each position. Differential protection of selected lysine residues was performed with (4,4-dimethyl-2,6-dioxocyclohex-1-ylidene)-3-methylbutyl (ivDde), and the ivDde group was selectively removed with 5% v/v hydrazine in DMF. The completed N-terminal residue on the main chain was protected with a t-butoxycarbonyl (Boc) protecting group.

Separately, a solution of bromoacetic acid (0.5 M in DCM, 20 eq) was carefully treated with DIC (1.0 M in DMF, 10 eq) and allowed to form the anhydride for 10 min. The entire solution was then added to the deprotected resin from above to install the bromoacetyl amide.

The completed peptide sequence was treated with a solution of trifluoroacetic acid/triisopropyl silane/water (TFA/TIS/H₂O=94:3:3) and stirred for 2 hours. The mixture was filtered, and the solution was partially dried by vacuum distillation. The peptide was precipitated with cyclopentylmethyl ether and centrifuged to pellet, and the solid was washed with methyl-t-butyl ether. After drying under vacuum overnight, the solid was dissolved in DMSO and purified by reverse-phase HPLC (Phenomenex Gemini®, 5 μm, 110 Å, 250×30 mm, A: 0.1% v/v TFA in water, B: 0.1% v/v TFA in ACN, gradient: 25%-40% v/v B over 60 min). The clean fractions of desired product were pooled, frozen, and lyophilized. The dried peptide was characterized for purity by LC/MS (Agilent 1290 HPLC equipped with a 6130 single-quad MS) and for peptide content by CLND (PAC).

Part 2: Antibody Conjugation with Peptide-Linker Molecule

Engineered antibodies with specific cysteine mutations were incubated with a solution of 1.0 mM cysteamine and 2.5 mM cystamine in 40 mM HEPES buffer (pH 8.2) at 2.5 mg/mL concentration for 15-20 hours. The reaction mixture was filtered through a 0.22 m polyethersulfone (PES) filter and diluted with 100 mM sodium acetate buffer pH 5.0. The reaction mixture was purified by cation exchange chromatography (Cytiva HiPrep™ 25 mL SP/HP, A: 20 mM NaOAc, pH 5.0, B: A+1.0 M NaCl, gradient: 0-30% v/v B over 10 CV, 5 mL/min). The main peak containing cysteamine-capped protein was collected and buffer exchanged to 10 mM sodium acetate with 9% w/v sucrose, pH 5.2 using MilliporeAmicon® Ultra-15 centrifugal concentrators (30 kDa membrane).

The above protein solution (6 mg/mL in 10 mM sodium acetate with 9% sucrose) was partially reduced using four equivalents of triphenylphosphine-3,3′,3′″-trisulfonic acid trisodium salt (TPPTS) at room temperature (RT) for 60-90 minutes. Reaction completion was determined by analytical cation exchange chromatography (YMC BioPro SP-F, 30×4.6 mm, A: 20 mM NaOAc, pH 5.0, B: A+1.0 M NaCl, 1.5 mL/min, Gradient: 10-30% B over 4 min). Excess TPPTS and reduction byproducts were removed by buffer exchange into clean 10 mM NaOAc, 9% sucrose, pH 5.2 using a centrifugal filter with a molecular weight cutoff at 30 kDa (MilliporeAmicon® Ultra-15). The reduced protein was diluted with 50 mM sodium phosphate buffer containing 2 mM ethylenediaminetetraacetic acid (EDTA) at pH 7.5 and treated with ten equivalents of dehydroascorbic acid (DHAA, BioSynth International) at RT until only trace amounts of partially reduced protein species were observed (30-120 minutes, monitored by RP-HPLC, Agilent PLRP-S, 4000 Å pore size, 5 μm particle size, 50×4.6 mm, A: 0.1% v/v TFA in H₂O, B: 0.1% v/v TFA in CH₃CN, gradient: 2-50% v/v B over 8 min). Without removing DHAA, 3-8 equivalents of bromoacetyl-glucagon-active peptide were added to the reaction mixture and incubated at RT for 15-20 hours. The completed reaction was typically purified by hydrophobic interaction chromatography (Cytiva HiTrap™ Butyl-HP, 5 mL; A: 1.0 M (NH₄)₂SO₄, 20 mM NaOAc, pH 5.0, B: 20 mM NaOAc, 10% v/v acetonitrile, pH 5.0, gradient: 0-100% v/v B over 50 min, 5.0 mL/min). The fractions containing desired product were pooled and buffer exchanged into 10 mM NaOAc, 9% w/v sucrose, pH 5.2. The material was characterized by HPLC-TOF-MS (Agilent 1260 HPLC equipped with a G6230B TOF-MS) and size exclusion chromatography (Agilent 1260 Bio-inert HPLC equipped with a Tosoh QC-Pak GFC 300 column).

Fc conjugates may be prepared by a similar process.

TABLE 19
GCGR Agonist Conjugate Constructs

| | | GCGR | | | | | | |
|---|---|---|---|---|---|---|---|---|
| | | Agonist | Peptide | Linker | HC | hGCGR | | |
| Conjugate | | SEQ ID | Attachment | SEQ ID | Attachment | EC₅₀ | hGIPR | hGLP-1R |
| No. | Description | NO: | Point | NO: | Point | (pM) | IC₅₀ (nM) | EC₅₀ (pM) |
| 16746-6 | aDNP SEFL2 | 1596 | K28 | 1629 | C273 | 79.5 | 0 | 27900 |
| | E384C-15997 | | | | | | | |
| 31214-1 | aDNP 3B1 SEFL2 | 1597 | K24 | 1629 | C278 | 52.1 | not tested | 136800 |
| | E384C-29137 | | | | | | | |
| 31215-1 | aDNP 3B1 SEFL2 | 1598 | K24 | 1629 | C278 | 62.1 | not tested | ~62020 |
| | E384C-29138 | | | | | | | |
| 31216-1 | aDNP 3B1 SEFL2 | 1599 | K28 | 1629 | C278 | 69.5 | not tested | 42350 |
| | E384C-29139 | | | | | | | |
| 31217-1 | aDNP 3B1 SEFL2 | 1600 | K28 | 1629 | C278 | 105.3 | not tested | 75290 |
| | E384C-29141 | | | | | | | |
| 31219-1 | aDNP 3B1 SEFL2 | 1601 | K28 | 1629 | C278 | 152.6 | not tested | 49620 |
| | E384C-29636 | | | | | | | |
| 31220-1 | aDNP 3B1 SEFL2 | 1602 | K28 | 1629 | C278 | 57.9 | not tested | 35010 |
| | E384C-29637 | | | | | | | |
| 31224-1 | aDNP 3B1 SEFL2 | 1603 | K28 | 1629 | C278 | 118.7 | not tested | 47460 |
| | E384C-29144 | | | | | | | |
| 34457-1 | aDNP 3B1 SEFL2 | 1604 | K28 | 1629 | C278 | 171.6 | not tested | 23300 |
| | E384C-31470 | | | | | | | |
| 34458-1 | aDNP 3B1 SEFL2 | 1605 | K28 | 1629 | C278 | 60.9 | not tested | 118500 |
| | E384C-31471 | | | | | | | |
| 34462-1 | aDNP 3B1 SEFL2 | 1588 | K28 | 1629 | C278 | 122.6 | not tested | 15500 |
| | E384C-31474 | | | | | | | |
| 34463-1 | aDNP 3B1 SEFL2 | 1606 | K28 | 1629 | C278 | 131.5 | not tested | 40930 |
| | E384C-32348 | | | | | | | |
| 34467-1 | aDNP 3B1 SEFL2 | 1607 | K28 | 1629 | C278 | 130.0 | not tested | 59630 |
| | E384C-32241 | | | | | | | |
| 34470-1 | aDNP 3B1 SEFL2 | 1608 | K28 | 1629 | C278 | 183.9 | not tested | 37750 |
| | E384C-32243 | | | | | | | |
| 34471-1 | aDNP 3B1 SEFL2 | 1609 | K28 | 1629 | C278 | 232.4 | not tested | 12750 |
| | E384C-32244 | | | | | | | |
| 35187-1 | aDNP 3B1 SEFL2 | 1879 | K28 | 1631 | C278 | 186.7 | not tested | 91510 |
| | E384C-31442 | | | | | | | |
| 35190-1 | aDNP 3B1 SEFL2 | 1611 | K28 | 1629 | C278 | 79.7 | not tested | 8934 |
| | E384C-32308 | | | | | | | |
| 36839-1 | aGIPR 5G12 SEFL2 | 1747 | K28 | 1629 | C276 | 123.1 | not tested | 43630 |
| | E384C-35881 | | | | | | | |
| 37468-1 | aGIPR 5G12 SEFL2 | 1603 | K28 | 1629 | C276 | 47.9 | not tested | 17340 |
| | E384C-29144 | | | | | | | |
| 39010-1 | aGIPR 5G12 SEFL2 | 1612 | K28 | 1629 | C276 | 24.7 | not tested | |
| | E384C-37059 | | | | | | | |
| 39011-1 | aGIPR 5G12 SEFL2 | 1613 | K24 | 1629 | C276 | 47.7 | not tested | 32040 |
| | E384C-36852 | | | | | | | |
| 39314-1 | aGIPR 5G12 SEFL2 | 1880 | K28 | 1629 | C276 | 58.1 | not tested | 50110 |
| | E384C-38864 | | | | | | | |
| 39316-1 | aGIPR 5G12 SEFL2 | 1615 | K28 | 1629 | C276 | 71.2 | 144.6 | 99060 |
| | E384C-38866 | | | | | | | |
| 39543-1 | aGIPR 5G12 SEFL2 | 1616 | K28 | 1629 | C276 | 27.8 | 103.1 | 40940 |
| | E384C-38867 | | | | | | | |
| 39546-1 | aGIPR 5G12 SEFL2 | 1617 | K28 | 1629 | C276 | 61.2 | not tested | 38970 |
| | E384C-38870 | | | | | | | |
| 39565-1 | aGIPR 5G12 SEFL2 | 1618 | K28 | 1629 | C276 | 71.3 | not tested | 19180 |
| | E384C-38871 | | | | | | | |
| 39584-1 | aGIPR 5G12 SEFL2 | 1619 | K28 | 1629 | C276 | 40.0 | 139.6 | 23860 |
| | E384C-38878 | | | | | | | |
| 39585-1 | aGIPR 5G12 SEFL2 | 1620 | K28 | 1629 | C276 | 52.4 | 113.7 | 44410 |
| | E384C-38879 | | | | | | | |
| 39988-1 | aGIPR 5G12 SEFL2 | 1588 | K28 | 1629 | C276 | 115.8 | 117.8 | 12590 |
| | E384C-31474 | | | | | | | |
| 40757-1 | aGIPR 5G12 SEFL2 | 1621 | K28 | 1629 | C276 | 22.8 | 171.0 | 21410 |
| | E384C-39983 | | | | | | | |
| 40759-1 | aGIPR 5G12 SEFL2 | 1622 | K28 | 1629 | C276 | 20.2 | 164.0 | 11590 |
| | E384C-39985 | | | | | | | |
| 40760-1 | aGIPR 5G12 SEFL2 | 1623 | K28 | 1629 | C276 | 26.6 | 195.4 | 4995 |
| | E384C-39890 | | | | | | | |
| 41871-1 | aGIPR 5G12 SEFL2 | 1624 | K28 | 1629 | C276 | 108.6 | not tested | 46540 |
| | E384C-41471 | | | | | | | |
| 41961-1 | aGIPR 5G12 SEFL2 | 1589 | K24 | 1629 | C276 | 68.7 | not tested | 5170 |
| | E384C-41683 | | | | | | | |
| 41962-1 | aGIPR 5G12 SEFL2 | 1592 | K24 | 1629 | C276 | 49 .. | not tested | 5471 |
| | E384C-41682 | | | | | | | |
| 41963-1 | aGIPR 5G12 SEFL2 | 1594 | K24 | 1629 | C276 | 135.1 | not tested | 86160 |
| | E384C-41681 | | | | | | | |
| 41964-1 | aGIPR 5G12 SEFL2 | 1590 | K28 | 1629 | C276 | 89.1 | not tested | 3650 |
| | E384C-41680 | | | | | | | |
| 41965-1 | aGIPR 5G12 SEFL2 | 1595 | K28 | 1629 | C276 | 106.2 | not tested | 27550 |
| | E384C-41679 | | | | | | | |
| 41966-1 | aGIPR 5G12 SEFL2 | 1881 | K24 | 1629 | C276 | 35.6 | not tested | 3411 |
| | E384C-41678 | | | | | | | |
| 41968-1 | aGIPR 5G12 SEFL2 | 1599 | K28 | 1629 | C276 | 52.3 | not tested | 5994 |
| | E384C-29139 | | | | | | | |
| 41972-1 | aGIPR 5G12 SEFL2 | 1596 | K28 | 1640 | C276 | 37.7 | not tested | 1754 |
| | E384C-41216 | | | | | | | |
| 41974-1 | aGIPR 5G12 SEFL2 | 1596 | K28 | 1628 | C276 | 172.6 | not tested | 13700 |
| | E384C-41218 | | | | | | | |
| 41975-1 | aGIPR 5G12 SEFL2 | 1596 | K28 | 1630 | C276 | 109.3 | not tested | 11020 |
| | E384C-41219 | | | | | | | |
| 41978-1 | aGIPR 5G12 SEFL2 | 1597 | K24 | 1640 | C276 | 62.5 | not tested | 9727 |
| | E384C-41477 | | | | | | | |
| 41979-1 | aGIPR 5G12 SEFL2 | 1597 | K24 | 1632 | C276 | 60.1 | not tested | 8566 |
| | E384C-41476 | | | | | | | |
| 41980-1 | aGIPR 5G12 SEFL2 | 1597 | K24 | 1628 | C276 | 50.3 | not tested | 3115 |
| | E384C-41475 | | | | | | | |
| 41981-1 | aGIPR 5G12 SEFL2 | 1597 | K24 | 1630 | C276 | 155.0 | not tested | 15710 |
| | E384C-41474 | | | | | | | |
| 41986-1 | aGIPR 5G12 SEFL2 | 1591 | K28 | 1629 | C276 | 86.2 | 162.1 | 47520 |
| | E384C-41684 | | | | | | | |
| 43161-1 | aGIPR 5G12 SEFL2 | 1596 | K28 | 1644 | C276 | 77.6 | not tested | 20880 |
| | E384C-42021 | | | | | | | |
| 43888-2 | aGIPR 5G12 SEFL2 | 1615 | K28 | 1630 | C276 | 79.7 | not tested | 90660 |
| | E384C-43779 | | | | | | | |
| 44499-1 | aGIPR 5G12 SEFL2 | 1626 | K28 | 1629 | C276 | 56.2 | 76.5 | 25220 |
| | E384C-44405 | | | | | | | |
| 44958-1 | aGIPR 5G12 SEFL2 | 1627 | K28 | 1628 | C276 | 26.8 | not tested | 40740 |
| | E384C-44868 | | | | | | | |
| 44959-1 | aGIPR 5G12 SEFL2 | 1626 | K28 | 1628 | C276 | 87.2 | 162.3 | 47330 |
| | E384C-44869 | | | | | | | |
| 44960-1 | aGIPR 5G12 SEFL2 | 1593 | K28 | 1628 | C276 | 37.7 | not tested | 41870 |
| | E384C-44870 | | | | | | | |
| 44961-1 | aGIPR 5G12 SEFL2 | 1592 | K24 | 1628 | C276 | 22.3 | 104.7 | 93700 |
| | E384C-44871 | | | | | | | |
| 44962-1 | aGIPR 5G12 SEFL2 | 1621 | K28 | 1628 | C276 | 37.0 | not tested | 5206000 |
| | E384C-44872 | | | | | | | |
| 44963-1 | aGIPR 5G12 SEFL2 | 1621 | K28 | 1630 | C276 | 60.4 | not tested | 680800 |
| | E384C-44873 | | | | | | | |
| 44964-1 | aGIPR 5G12 SEFL2 | 1615 | K28 | 1628 | C276 | 27.8 | not tested | 99610 |
| | E384C-44874 | | | | | | | |
| 44965-1 | aGIPR 5G12 SEFL2 | 1622 | K28 | 1630 | C276 | 51.5 | not tested | 128900 |
| | E384C-45025 | | | | | | | |
| 44966-1 | aGIPR 5G12 SEFL2 | 1622 | K28 | 1628 | C276 | 24.7 | not tested | 42360 |
| | E384C-44875 | | | | | | | |
| 45030-4 | aGIPR 2G10 SEFL2 | 1592 | K24 | 1628 | C275 | 44.9 | 88.9 | 88380 |
| | E384C-44871 | | | | | | | |
| 45053-1 | aGIPR 5G12 SEFL2 | 1627 | K28 | 1630 | C276 | 52.6 | not tested | 72290 |
| | E384C-45023 | | | | | | | |
| 45055-1 | aGIPR 5G12 SEFL2 | 1626 | K28 | 1630 | C276 | 63.4 | not tested | 79260 |
| | E384C-45024 | | | | | | | |
| 45056-1 | aGIPR 5G12 SEFL2 | 1593 | K28 | 1630 | C276 | 47.3 | not tested | 25170 |
| | E384C-44876 | | | | | | | |
| 45333-1 | aGIPR 2G10 SEFL2 | 1615 | K28 | 1629 | C275 | 68.0 | 51.9 | 58850 |
| | E384C-38866 | | | | | | | |
| 46172-4 | aGIPR 2G10 SEFL2 | 1626 | K28 | 1628 | C275 | 63.4 | 37.4 | 57540 |
| | E384C-44869 | | | | | | | |
| 50705-1 | aGIPR 2G10 SEFL2 | 1626 | K28 | 1629 | C275 | 37.9 | 29.1 | 39390 |
| | E384C-44405 | | | | | | | |
| 51671-1 | aGIPR 2G10 SEFL2 | 1587 | K28 | 1628 | C275 | 56.1 | 7.8 | 23690 |
| | E384C-50927 | | | | | | | |
| 51672-1 | aGIPR 2G10 SEFL2 | 1587 | K28 | 1630 | C275 | 236.4 | 92.1 | 17230 |
| | E384C-51493 | | | | | | | |
| 52398-1 | aGIPR 5G12 SEFL2 | 1587 | K28 | 1628 | C276 | 109.6 | 71.3 | 38850 |
| | E384C-50927 | | | | | | | |
| 52399-1 | aGIPR 5G12 SEFL2 | 1587 | K28 | 1630 | C276 | 452.4 | 162.2 | 50010 |
| | E384C-51493 | | | | | | | |
| 1658 | aGIPR 2G10.007 | 1859 | K28 | 1639 | C275 | not tested | not tested | not tested |
| | SEFL2-E384C- | | | | | | | |
| | 3358060 | | | | | | | |
| 1659 | aGIPR 2G10.007 | 1860 | K21 | 1639 | C275 | not tested | not tested | not tested |
| | SEFL2-E384C- | | | | | | | |
| | 3358061 | | | | | | | |
| 1898 | aGIPR 2G10.007 | 1859 | K28 | 1629 | C275 | 704.8 | not tested | 9616.0 |
| | SEFL2-E384C- | | | | | | | |
| | 3360331 | | | | | | | |
| 16745 | aDNP SEFL2 E384C- | 1748 | K28 | 1629 | C273 | not tested | not tested | not tested |
| | 15996 | | | | | | | |
| 24998 | aDNP SEFL2 E384C- | 1750 | Azk28 | 1739 | C273 | 28.0 | not tested | not tested |
| | 15997-triazole | | | | | | | |
| 25267 | 655-341 SEFL2 | 1596 | K28 | 1629 | C364 | 16.8 | not tested | 4065.0 |
| | T487C-15997 | | | | | | | |
| 25289 | 655-341 SEFL2 | 1596 | K28 | 1629 | C277 | 28.3 | 0.0 | 25770.0 |
| | E384C-15997 | | | | | | | |
| 25435 | Fc SEFL2 T487C- | 1596 | K28 | 1629 | C139 | 9.1 | not tested | 9770.0 |
| | 15997 | | | | | | | |
| 25444 | aDNP 3B1 SEFL2 | 1596 | K28 | 1629 | C278 | 53.1 | 0.0 | 25500.0 |
| | E384C-15997 | | | | | | | |
| 25445 | aGIPR 2G10.007 | 1596 | K28 | 1629 | C275 | 51.5 | curve not | 34680.0 |
| | SEFL2-E384C- | | | | | | converged | |
| | 15997 | | | | | | | |
| 25458 | aGIPR 5G12 SEFL2 | 1596 | K28 | 1629 | C276 | 32.4 | 223.7 | 20290.0 |
| | E384C-15997 | | | | | | | |
| 25653 | aDNP SEFL2 E384C- | 1749 | K28 | 1629 | C273 | 16.9 | not tested | 1539.0 |
| | 23775 | | | | | | | |
| 25675 | aGIPR 5G12 SEFL2 | 1596 | K28 | 1629 | C363 | 16.5 | not tested | 10960.0 |
| | T487C-15997 | | | | | | | |
| 25676 | 655-341 SEFL2 D88C- | 1596 | K28 | 1629 | C71 | 12.8 | not tested | 7034.0 |
| | 15997 | | | | | | | |
| 26917 | aDNP SEFL2 E384C- | 1751 | K28 | 1629 | C273 | 149.9 | not tested | 7957.0 |
| | 25278 | | | | | | | |
| 31226 | aDNP 3B1 SEFL2 | 1764 | K28 | 1631 | C278 | 354.0 | not tested | 146300.0 |
| | E384C-30796 | | | | | | | |
| 34465 | aDNP 3B1 SEFL2 | 1766 | K28 | 1629 | C278 | 613.1 | not tested | 57800.0 |
| | E384C-32350 | | | | | | | |
| 35188 | aDNP 3B1 SEFL2 | 1764 | K28 | 1629 | C278 | 347.9 | not tested | 79520.0 |
| | E384C-32319 | | | | | | | |
| 35191 | aDNP 3B1 SEFL2 | 1765 | K28 | 1629 | C278 | 176.2 | not tested | 141100.0 |
| | E384C-32307 | | | | | | | |
| 36799 | aGIPR 5G12 SEFL2 | 1767 | K24 | 1629 | C276 | 26.6 | not tested | 0.0 |
| | E384C-35507 | | | | | | | |
| 36801 | aGIPR 5G12 SEFL2 | 1861 | K28 | 1629 | C276 | 124.3 | not tested | 0.0 |
| | E384C-35509 | | | | | | | |
| 36803 | aGIPR 5G12 SEFL2 | 1768 | K24 | 1629 | C276 | 51.4 | not tested | ~70460 |
| | E384C-35510 | | | | | | | |
| 36805 | aGIPR 5G12 SEFL2 | 1769 | K28 | 1629 | C276 | 74.0 | not tested | ~4.5e15 |
| | E384C-35512 | | | | | | | |
| 36834 | aGIPR 5G12 SEFL2 | 1770 | K28 | 1629 | C276 | 36.8 | not tested | 9530.0 |
| | E384C-35515 | | | | | | | |
| 36835 | aGIPR 5G12 SEFL2 | 1771 | K28 | 1629 | C276 | 263.0 | not tested | 17350.0 |
| | E384C-35516 | | | | | | | |
| 36836 | aGIPR 5G12 SEFL2 | 1862 | K28 | 1629 | C276 | 1050.0 | not tested | 188400.0 |
| | E384C-35878 | | | | | | | |
| 36837 | aGIPR 5G12 SEFL2 | 1772 | K28 | 1629 | C276 | 620.2 | not tested | ~76670 |
| | E384C-35879 | | | | | | | |
| 36838 | aGIPR 5G12 SEFL2 | 1773 | K28 | 1629 | C276 | 721.0 | not tested | ~323300000 |
| | E384C-35880 | | | | | | | |
| 37467 | aGIPR 5G12 SEFL2 | 1597 | K24 | 1629 | C276 | 24.5 | not tested | 52500.0 |
| | E384C-29137 | | | | | | | |
| 39009 | aGIPR 5G12 SEFL2 | 1774 | K28 | 1629 | C276 | 57.1 | not tested | 0.0 |
| | E384C-37058 | | | | | | | |
| 39315 | aGIPR 5G12 SEFL2 | 1840 | K28 | 1629 | C276 | 191.2 | not tested | 88780.0 |
| | E384C-38865 | | | | | | | |
| 39544 | aGIPR 5G12 SEFL2 | 1775 | K28 | 1629 | C276 | 87.6 | not tested | 17140.0 |
| | E384C-38868 | | | | | | | |
| 39545 | aGIPR 5G12 SEFL2 | 1776 | K28 | 1629 | C276 | 92.6 | not tested | 28710.0 |
| | E384C-38869 | | | | | | | |
| 39578 | aGIPR 5G12 SEFL2 | 1763 | K28 | 1629 | C276 | 155.0 | not tested | 10260.0 |
| | E384C-38872 | | | | | | | |
| 39579 | aGIPR 5G12 SEFL2 | 1777 | K28 | 1629 | C276 | 80.0 | not tested | 49090.0 |
| | E384C-38873 | | | | | | | |
| 39580 | aGIPR 5G12 SEFL2 | 1778 | K28 | 1629 | C276 | 74.6 | not tested | 38000.0 |
| | E384C-38874 | | | | | | | |
| 39581 | aGIPR 5G12 SEFL2 | 1779 | K28 | 1629 | C276 | 98.0 | not tested | 40390.0 |
| | E384C-38875 | | | | | | | |
| 39582 | aGIPR 5G12 SEFL2 | 1780 | K28 | 1629 | C276 | 174.4 | not tested | 152500.0 |
| | E384C-38876 | | | | | | | |
| 39583 | aGIPR 5G12 SEFL2 | 1781 | K28 | 1629 | C276 | 113.6 | not tested | 52330.0 |
| | E384C-38877 | | | | | | | |
| 39586 | aGIPR 5G12 SEFL2 | 1782 | K28 | 1629 | C276 | 366.6 | not tested | 15970.0 |
| | E384C-38880 | | | | | | | |
| 39587 | aGIPR 5G12 SEFL2 | 1783 | K28 | 1629 | C276 | 108.2 | not tested | 39080.0 |
| | E384C-38881 | | | | | | | |
| 40741 | aGIPR 5G12 SEFL2 | 1784 | K28 | 1629 | C276 | 40.9 | not tested | 8015.0 |
| | E384C-39903 | | | | | | | |
| 40743 | aGIPR 5G12 SEFL2 | 1755 | K28 | 1629 | C276 | 31.5 | not tested | 5028.0 |
| | E384C-39906 | | | | | | | |
| 40744 | aGIPR 5G12 SEFL2 | 1785 | K28 | 1629 | C276 | 122.5 | not tested | 8068.0 |
| | E384C-39907 | | | | | | | |
| 40745 | aGIPR 5G12 SEFL2 | 1786 | K28 | 1629 | C276 | 28.2 | not tested | 4634.0 |
| | E384C-39908 | | | | | | | |
| 40746 | aGIPR 5G12 SEFL2 | 1787 | K24 | 1629 | C276 | 17.3 | not tested | ~3821000 |
| | E384C-39909 | | | | | | | |
| 40747 | aGIPR 5G12 SEFL2 | 1791 | K28 | 1629 | C276 | 21.1 | not tested | 73570.0 |
| | E384C-39981 | | | | | | | |
| 40749 | aGIPR 5G12 SEFL2 | 1788 | K24 | 1629 | C276 | 15.4 | not tested | 7916.0 |
| | E384C-39912 | | | | | | | |
| 40752 | aGIPR 5G12 SEFL2 | 1762 | K24 | 1629 | C276 | 22.3 | not tested | 3260.0 |
| | E384C-39915 | | | | | | | |
| 40754 | aGIPR 5G12 SEFL2 | 1789 | K31 | 1629 | C276 | 81.5 | not tested | 5471.0 |
| | E384C-39917 | | | | | | | |
| 40755 | aGIPR 5G12 SEFL2 | 1792 | K28 | 1629 | C276 | 151.0 | not tested | 15010.0 |
| | E384C-39982 | | | | | | | |
| 40758 | aGIPR 5G12 SEFL2 | 1794 | K28 | 1629 | C276 | 70.5 | not tested | 16630.0 |
| | E384C-39984 | | | | | | | |
| 41383 | aGIPR 5G12 SEFL2 | 1754 | K28 | 1629 | C276 | 129.1 | not tested | 183000.0 |
| | E384C-40387 | | | | | | | |
| 41385 | aGIPR 5G12 SEFL2 | 1795 | K28 | 1629 | C276 | 151.7 | not tested | 42870.0 |
| | E384C-40389 | | | | | | | |
| 41389 | aGIPR 5G12 SEFL2 | 1796 | K28 | 1629 | C276 | 44.7 | not tested | 226100.0 |
| | E384C-40835 | | | | | | | |
| 41390 | aGIPR 5G12 SEFL2 | 1790 | K28 | 1629 | C276 | 59.7 | not tested | 223000.0 |
| | E384C-39920 | | | | | | | |
| 41391 | aGIPR 5G12 SEFL2 | 1797 | K24 | 1629 | C276 | 53.3 | not tested | ~69750 |
| | E384C-40837 | | | | | | | |
| 41967 | aGIPR 5G12 SEFL2 | 1798 | K24 | 1629 | C276 | 86.2 | not tested | 38000.0 |
| | E384C-41677 | | | | | | | |
| 41973 | aGIPR 5G12 SEFL2 | 1596 | K28 | 1632 | C276 | 185.6 | not tested | 10460.0 |
| | E384C-41217 | | | | | | | |
| 41977 | aGIPR 5G12 SEFL2 | 1597 | K24 | 1634 | C276 | 139.7 | not tested | 11020.0 |
| | E384C-41478 | | | | | | | |
| 41982 | aGIPR 5G12 SEFL2 | 1597 | K24 | 1641 | C276 | 167.5 | not tested | 24370.0 |
| | E384C-41687 | | | | | | | |
| 41984 | aGIPR 5G12 SEFL2 | 1597 | K24 | 1642 | C276 | 56.6 | not tested | 21550.0 |
| | E384C-41686 | | | | | | | |
| 41985 | aGIPR 5G12 SEFL2 | 1596 | K28 | 1642 | C276 | 76.3 | not tested | 9559.0 |
| | E384C-41685 | | | | | | | |
| 42137 | aGIPR 5G12 SEFL2 | 1757 | K28 | 1629 | C276 | 58.4 | not tested | 11070.0 |
| | E384C-41902 | | | | | | | |
| 42138 | aGIPR 5G12 SEFL2 | 1756 | K28 | 1629 | C276 | 83.2 | not tested | 26850.0 |
| | E384C-41903 | | | | | | | |
| 42139 | aGIPR 5G12 SEFL2 | 1759 | K28 | 1629 | C276 | 80.5 | not tested | 49840.0 |
| | E384C-41904 | | | | | | | |
| 42140 | aGIPR 5G12 SEFL2 | 1758 | K28 | 1629 | C276 | 76.7 | not tested | 54700.0 |
| | E384C-41905 | | | | | | | |
| 42141 | aGIPR 5G12 SEFL2 | 1760 | K28 | 1629 | C276 | 33.0 | not tested | 26710.0 |
| | E384C-41906 | | | | | | | |
| 42142 | aGIPR 5G12 SEFL2 | 1761 | K28 | 1629 | C276 | 72.1 | not tested | 64190.0 |
| | E384C-41907 | | | | | | | |
| 43142 | aGIPR 5G12 SEFL2 | 1596 | K28 | 1635 | C276 | 335.5 | not tested | 38910.0 |
| | E384C-42020 | | | | | | | |
| 43162 | aGIPR 5G12 SEFL2 | 1596 | K28 | 1647 | C276 | 77.8 | not tested | 30790.0 |
| | E384C-42022 | | | | | | | |
| 43163 | aGIPR 5G12 SEFL2 | 1597 | K24 | 1644 | C276 | 51.7 | not tested | 219600.0 |
| | E384C-42023 | | | | | | | |
| 43164 | aGIPR 5G12 SEFL2 | 1597 | K24 | 1647 | C276 | 50.9 | not tested | 196900.0 |
| | E384C-42024 | | | | | | | |
| 43166 | aGIPR 5G12 SEFL2 | 1799 | C-term | 1742 | C276 | 48.9 | not tested | 2056.0 |
| | E384C-42026 | | | | | | | |
| 43167 | aGIPR 5G12 SEFL2 | 1800 | C-term | 1845 | C276 | 27.7 | not tested | 10800.0 |
| | E384C-42027 | | | | | | | |
| 43168 | aGIPR 5G12 SEFL2 | 1800 | C-term | 1846 | C276 | 59.0 | not tested | 14590.0 |
| | E384C-42028 | | | | | | | |
| 43464 | aGIPR 5G12 SEFL2 | 1753 | K28 | 1629 | C276 | 66.3 | not tested | 4292.0 |
| | E384C-42175 | | | | | | | |
| 43465 | aGIPR 5G12 SEFL2 | 1808 | K28 | 1629 | C276 | 46.0 | not tested | 28720.0 |
| | E384C-42176 | | | | | | | |
| 43466 | aGIPR 5G12 SEFL2 | 1809 | K28 | 1629 | C276 | 60.1 | not tested | 9295.0 |
| | E384C-42177 | | | | | | | |
| 43467 | aGIPR 5G12 SEFL2 | 1810 | K28 | 1629 | C276 | 58.0 | not tested | 5946.0 |
| | E384C-42178 | | | | | | | |
| 43468 | aGIPR 5G12 SEFL2 | 1811 | K24 | 1629 | C276 | 25.4 | not tested | 48570.0 |
| | E384C-42179 | | | | | | | |
| 43469 | aGIPR 5G12 SEFL2 | 1812 | K24 | 1629 | C276 | 55.0 | not tested | 23660.0 |
| | E384C-42180 | | | | | | | |
| 43470 | aGIPR 5G12 SEFL2 | 1813 | K28 | 1629 | C276 | 77.0 | not tested | ~1663000000 |
| | E384C-42181 | | | | | | | |
| 43471 | aGIPR 5G12 SEFL2 | 1814 | K28 | 1629 | C276 | 38.3 | not tested | 152800.0 |
| | E384C-42182 | | | | | | | |
| 43472 | aGIPR 5G12 SEFL2 | 1815 | K28 | 1629 | C276 | 63.8 | not tested | 17790.0 |
| | E384C-42183 | | | | | | | |
| 43473 | aGIPR 5G12 SEFL2 | 1799 | C-term | 1849 | C276 | 84.7 | not tested | 3471.0 |
| | E384C-42193 | | | | | | | |
| 43474 | aGIPR 5G12 SEFL2 | 1799 | C-term | 1848 | C276 | 52.1 | not tested | 3298.0 |
| | E384C-42194 | | | | | | | |
| 43475 | aGIPR 5G12 SEFL2 | 1788 | K24 | 1629 | C276 | 21.5 | not tested | 44220.0 |
| | E384C-42127 | | | | | | | |
| 43476 | aGIPR 5G12 SEFL2 | 1801 | K28 | 1629 | C276 | 21.7 | not tested | 6572.0 |
| | E384C-42128 | | | | | | | |
| 43477 | aGIPR 5G12 SEFL2 | 1802 | K28 | 1629 | C276 | 37.0 | not tested | 10400.0 |
| | E384C-42129 | | | | | | | |
| 43478 | aGIPR 5G12 SEFL2 | 1803 | K28 | 1629 | C276 | 276.9 | not tested | ~63140 |
| | E384C-42130 | | | | | | | |
| 43479 | aGIPR 5G12 SEFL2 | 1804 | K24 | 1629 | C276 | 108.4 | not tested | 1163.0 |
| | E384C-42131 | | | | | | | |
| 43480 | aGIPR 5G12 SEFL2 | 1805 | K24 | 1629 | C276 | 34.3 | not tested | 3423.0 |
| | E384C-42132 | | | | | | | |
| 43481 | aGIPR 5G12 SEFL2 | 1806 | K28 | 1629 | C276 | 35.3 | not tested | 21330.0 |
| | E384C-42133 | | | | | | | |
| 43483 | aGIPR 5G12 SEFL2 | 1807 | K28 | 1629 | C276 | 39.4 | not tested | 16640.0 |
| | E384C-42135 | | | | | | | |
| 43911 | aGIPR 5G12 SEFL2 | 1615 | K28 | 1638 | C276 | 261.4 | not tested | 49230.0 |
| | E384C-43818 | | | | | | | |
| 43912 | aGIPR 5G12 SEFL2 | 1615 | K28 | 1640 | C276 | 92.0 | not tested | 53680.0 |
| | E384C-43817 | | | | | | | |
| 44496 | aGIPR 5G12 SEFL2 | 1816 | K28 | 1629 | C276 | 85.5 | not tested | 22450.0 |
| | E384C-44402 | | | | | | | |
| 44497 | aGIPR 5G12 SEFL2 | 1817 | K28 | 1629 | C276 | 101.2 | not tested | 15820.0 |
| | E384C-44403 | | | | | | | |
| 44498 | aGIPR 5G12 SEFL2 | 1627 | K28 | 1629 | C276 | 50.1 | not tested | 21610.0 |
| | E384C-44404 | | | | | | | |
| 44500 | aGIPR 5G12 SEFL2 | 1818 | K28 | 1629 | C276 | 46.6 | not tested | 12540.0 |
| | E384C-44406 | | | | | | | |
| 44501 | aGIPR 5G12 SEFL2 | 1819 | K28 | 1629 | C276 | 33.2 | not tested | 21790.0 |
| | E384C-44407 | | | | | | | |
| 44502 | aGIPR 5G12 SEFL2 | 1820 | K28 | 1629 | C276 | 70.6 | not tested | 21030.0 |
| | E384C-44408 | | | | | | | |
| 44503 | aGIPR 5G12 SEFL2 | 1593 | K28 | 1629 | C276 | 49.1 | not tested | 190700.0 |
| | E384C-44409 | | | | | | | |
| 44984 | aGIPR 2G10 SEFL2 | 1593 | K28 | 1630 | C275 | 139.2 | 33.8 | 46010.0 |
| | E384C-44876 | | | | | | | |
| 45027 | aGIPR 2G10 SEFL2 | 1621 | K28 | 1628 | C275 | 78.5 | 86.9 | 175600.0 |
| | E384C-44872 | | | | | | | |
| 45028 | aGIPR 2G10 SEFL2 | 1621 | K28 | 1630 | C275 | 166.5 | 58.2 | 40220.0 |
| | E384C-44873 | | | | | | | |
| 45029 | aGIPR 2G10 SEFL2 | 1593 | K28 | 1628 | C275 | 69.8 | 49.3 | 28870.0 |
| | E384C-44870 | | | | | | | |
| 45034 | aGIPR 2G10 SEFL2 | 1615 | K28 | 1628 | C275 | 89.9 | 47.9 | 69310.0 |
| | E384C-44874 | | | | | | | |
| 45057 | aGIPR 5G12 SEFL2 | 1793 | C-term | 1741 | C276 | 50.4 | not tested | 5192.0 |
| | E384C-45022 | | | | | | | |
| 45332 | aGIPR 2G10 SEFL2 | 1621 | K28 | 1629 | C275 | 29.7 | 104.6 | 30100.0 |
| | E384C-39983 | | | | | | | |
| 45591 | aGIPR 2G10 SEFL2 | 1622 | K28 | 1628 | C275 | 97.7 | 32.9 | 118100.0 |
| | E384C-44875 | | | | | | | |
| 45592 | aGIPR 2G10 SEFL2 | 1627 | K28 | 1628 | C275 | 75.1 | 67.3 | 56480.0 |
| | E384C-44868 | | | | | | | |
| 46147 | aGIPR 2G10 SEFL2 | 1622 | K28 | 1630 | C275 | 67.2 | 102.4 | 26830.0 |
| | E384C-45025 | | | | | | | |
| 46148 | aGIPR 2G10 SEFL2 | 1627 | K28 | 1630 | C275 | 123.8 | 60.0 | 43160.0 |
| | E384C-45023 | | | | | | | |
| 46149 | aGIPR 2G10 SEFL2 | 1615 | K28 | 1630 | C275 | 291.0 | 80.9 | 63310.0 |
| | E384C-43779 | | | | | | | |
| 46173 | aGIPR 2G10 SEFL2 | 1626 | K28 | 1630 | C275 | 113.3 | 63.1 | 72600.0 |
| | E384C-45024 | | | | | | | |
| 46174 | aGIPR 2G10 SEFL2 | 1622 | K28 | 1629 | C275 | 62.5 | 80.5 | 40170.0 |
| | E384C-39985 | | | | | | | |
| 46175 | aGIPR 2G10 SEFL2 | 1793 | C-term | 1741 | C275 | 190.8 | 115.1 | 12170.0 |
| | E384C-45022 | | | | | | | |
| 60883 | aDNP SEFL2 | 1587 | K28 | 1628 | C273 | 32.5 | ~181.0 | 11690.0 |
| | E384C-50927 | | | | | | | |
| 60894 | aDNP SEFL2 | 1626 | K28 | 1628 | C273 | 42.2 | ~281.8 | 42230.0 |
| | E384C-44869 | | | | | | | |
| 60895 | aDNP SEFL2 | 1626 | K28 | 1629 | C273 | 21.7 | ~543.3 | 25030.0 |
| | E384C-44405 | | | | | | | |
| 88006 | aGIPR 5G12 SEFL2 | 1821 | K24 | 1628 | C276 | 75.4 | not tested | not tested |
| | E384C-86873 | | | | | | | |
| 88007 | aGIPR 5G12 SEFL2 | 1822 | K24 | 1628 | C276 | 30.9 | not tested | not tested |
| | E384C-86874 | | | | | | | |
| 88008 | aGIPR 5G12 SEFL2 | 1819 | K28 | 1628 | C276 | 56.1 | not tested | not tested |
| | E384C-87586 | | | | | | | |
| 88009 | aGIPR 5G12 SEFL2 | 1823 | K28 | 1628 | C276 | 44.4 | not tested | not tested |
| | E384C-86875 | | | | | | | |
| 88010 | aGIPR 5G12 SEFL2 | 1824 | K28 | 1628 | C276 | 37.7 | not tested | not tested |
| | E384C-86876 | | | | | | | |
| 88011 | aGIPR 5G12 SEFL2 | 1825 | K28 | 1628 | C276 | 64.9 | not tested | not tested |
| | E384C-87020 | | | | | | | |
| 88012 | aGIPR 5G12 SEFL2 | 1826 | K28 | 1628 | C276 | 51.6 | not tested | not tested |
| | E384C-87588 | | | | | | | |
| 88013 | aGIPR 5G12 SEFL2 | 1752 | K28 | 1628 | C276 | 41.3 | not tested | not tested |
| | E384C-87021 | | | | | | | |
| 88014 | aGIPR 5G12 SEFL2 | 1793 | C-term | 1841 | C276 | 86.1 | not tested | not tested |
| | E384C-86881 | | | | | | | |
| 88016 | aGIPR 5G12 SEFL2 | 1818 | K28 | 1628 | C276 | 37.2 | not tested | not tested |
| | E384C-87587 | | | | | | | |
| 89982 | aGIPR 2G10 SEFL2 | 1818 | K28 | 1628 | C275 | not tested | not tested | not tested |
| | E384C-87587 | | | | | | | |
| 89983 | aGIPR 2G10 SEFL2 | 1826 | K28 | 1628 | C275 | not tested | not tested | not tested |
| | E384C-87588 | | | | | | | |
| 89984 | aGIPR 2G10 SEFL2 | 1822 | K24 | 1628 | C275 | not tested | not tested | not tested |
| | E384C-86874 | | | | | | | |
| 89985 | aGIPR 2G10 SEFL2 | 1825 | K28 | 1628 | C275 | not tested | not tested | not tested |
| | E384C-87020 | | | | | | | |
| 90658 | aGIPR 5G12 SEFL2 | 1825 | K28 | 1628 | C363 | 40.2 | not tested | 8250.0 |
| | T487C-87020 | | | | | | | |
| 91657 | aGIPR 5G12 SEFL2 | 1827 | K28 | 1628 | C276 | 153.5 | not tested | 53990.0 |
| | E384C-91253 | | | | | | | |
| 91658 | aGIPR 5G12 SEFL2 | 1828 | K28 | 1628 | C276 | 185.7 | not tested | 1963.0 |
| | E384C-91254 | | | | | | | |
| 91659 | aGIPR 5G12 SEFL2 | 1829 | K28 | 1628 | C276 | 152.3 | not tested | 6754.0 |
| | E384C-91255 | | | | | | | |
| 91660 | aGIPR 5G12 SEFL2 | 1825 | K28 | 1628 | C276 | 283.3 | not tested | 2072.0 |
| | E384C-91256 | | | | | | | |
| 91661 | aGIPR 5G12 SEFL2 | 1830 | K28 | 1628 | C276 | 118.9 | not tested | 6346.0 |
| | E384C-91257 | | | | | | | |
| 91662 | aGIPR 5G12 SEFL2 | 1831 | C-term | 1842 | C276 | 286.7 | not tested | 3398.0 |
| | E384C-91258 | | | | | | | |
| 91663 | aGIPR 5G12 SEFL2 | 1831 | C-term | 1847 | C276 | 80.8 | not tested | 1475.0 |
| | E384C-91259 | | | | | | | |
| 91664 | aGIPR 5G12 SEFL2 | 1832 | C-term | 1842 | C276 | 95.9 | not tested | 12420.0 |
| | E384C-91260 | | | | | | | |
| 91665 | aGIPR 5G12 SEFL2 | 1831 | C-term | 1843 | C276 | 127.5 | not tested | 3585.0 |
| | E384C-91261 | | | | | | | |
| 91666 | aGIPR 5G12 SEFL2 | 1831 | C-term | 1844 | C276 | 152.8 | not tested | 5052.0 |
| | E384C-91262 | | | | | | | |
| 91667 | aGIPR 5G12 SEFL2 | 1833 | K24 | 1628 | C276 | 353.1 | not tested | 63400.0 |
| | E384C-91656 | | | | | | | |
| 92868 | aGIPR 5G12 SEFL2 | 1834 | K28 | 1628 | C276 | 127.2 | not tested | 289.0 |
| | E384C-92840 | | | | | | | |
| 92874 | aGIPR 5G12 SEFL2 | 1835 | K28 | 1628 | C276 | 13350.0 | not tested | 6379.0 |
| | E384C-92841 | | | | | | | |
| 93070 | aGIPR 5G12 SEFL2 | 1836 | K28 | 1628 | C276 | 146.7 | not tested | 1983.0 |
| | E384C-92842 | | | | | | | |
| 93071 | aGIPR 5G12 SEFL2 | 1837 | K24 | 1628 | C276 | 127.3 | not tested | 22970.0 |
| | E384C-92843 | | | | | | | |
| 93072 | aGIPR 5G12 SEFL2 | 1838 | K24 | 1628 | C276 | 110.5 | not tested | 16380.0 |
| | E384C-92847 | | | | | | | |

### Section 2: In Vitro Characterization of GCGR Agonist Conjugate Constructs

### Example 1: GCG Receptor Agonist Activity

CHOK1 cells stably expressing human GCGR and primary human and mouse hepatocytes with endogenous levels of GCGR were used to measure conjugate-induced cAMP production in a homogeneous time-resolved fluorescence (HTRF) assay (Cisbio, Catalog No. 62AM4PEJ). Serial diluted conjugates were incubated with 40,000 cells in assay buffer (0.1% v/v BSA, 500 pM IBMX in F12 media) for 15 min at 37° C. Cells were then lysed with lysis buffer containing cAMP-d2 and cAMP cryptate (Cisbio) and incubated for 1 hour at room temperature before fluorescence was measured in an EnVision® plate reader (PerkinElmer).

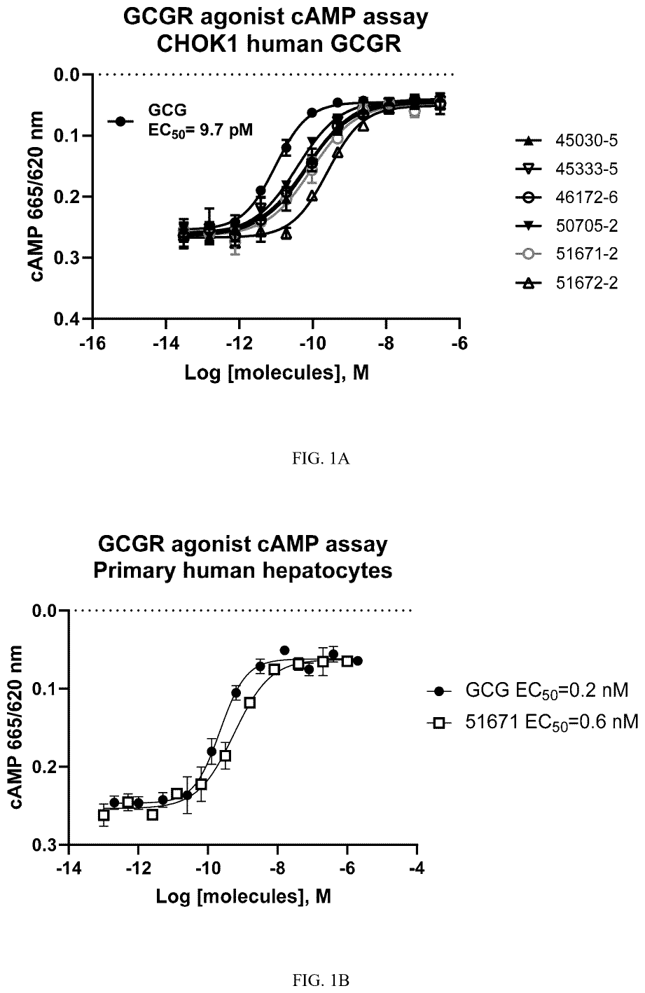

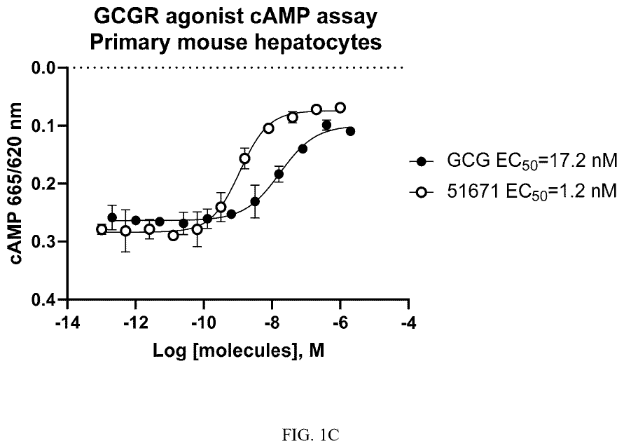

cAMP levels for six conjugates (45030-5, 45333-5, 46172-6, 50705-2, 51671-2, and 51672-2) and a positive control (recombinant human glucagon (GCG)) are expressed as a fluorescence ratio of 665/620 nm and shown in FIG. 1A for the CHOK1 cells stably expressing human GCGR.

Additionally, cAMP levels for conjugate 51671 and a positive control (recombinant human GCG) are shown in FIG. 1B for the primary human hepatocytes expressing human GCGR, where each data point represents the mean of two biological replicates±standard deviation. In addition, cAMP levels for conjugate 51671 and a positive control (recombinant mouse GCG) are shown in FIG. 1C for the primary mouse hepatocytes expressing mouse GCGR, where each data point represents the mean of two biological replicates±standard deviation.

EC₅₀ values for human GCGR determined using this functional assay with CHOK1 cells stably expressing human GCGR are provided for example conjugates in Table 19.

### Example 2: GLP-1 Receptor Agonist Activity

The GCGR agonist conjugates disclosed herein are highly selective for the glucagon receptor relative to the GLP-1 receptor. To assess GLP-1 receptor agonist activity for the conjugates, CHOK1 cells stably expressing human GLP-1R were used to measure conjugate-induced cAMP production in a homogeneous time-resolved fluorescence (HTRF) assay (Cisbio, Catalog No. 62AM4PEJ). Serial diluted conjugates were incubated with 40,000 cells in assay buffer (0.1% BSA, 500 pM IBMX in F12 media) for 15 min at 37° C. Cells were then lysed with lysis buffer containing cAMP-d2 and cAMP cryptate (Cisbio) and incubated for 1 hour at room temperature before fluorescence was measured in an EnVision® plate reader (PerkinElmer).

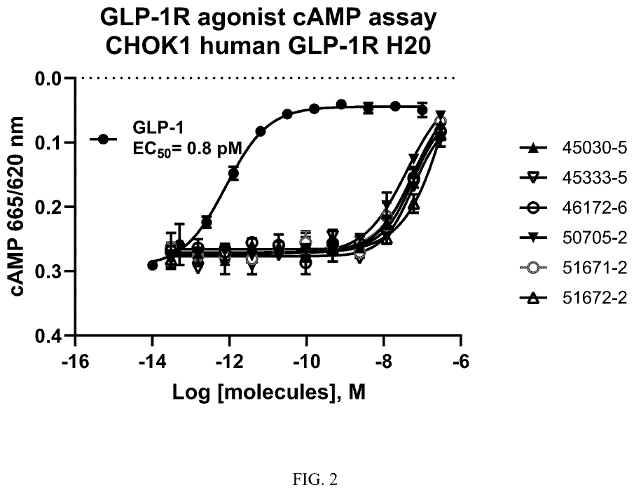

Representative cAMP levels for six conjugates (45030-5, 45333-5, 46172-6, 50705-2, 51671-2, and 51672-2) and a positive control (recombinant human GLP-1) are expressed as a fluorescence ratio of 665/620 nm and shown in FIG. 2.

Additionally, EC₅₀ values for human GLP-1 receptor determined using this functional assay are provided for example conjugates in Table 19.

### Example 3: GIPR Antagonist Activity

HEK 293T cells stably expressing human GIPR and CHO AM1D cells expressing mouse GIPR were used to measure conjugate-induced cAMP generation in a HTRF assay (Cisbio, Catalog No. 62AM4PEJ). Serial diluted conjugates were incubated with 30,000 cells in assay buffer (0.1% BSA, 500 pM IBMX in F12 media) for 30 min at 37° C. before being treated with GIP at final concentration of 50 pM (human GIP) in the HEK 293T human GIPR cells and 90 pM in the CHO AM1D mouse GIPR cells (mouse GIP). Cells were incubated for an additional 30 min at 37° C. and then lysed in lysis buffer containing cAMP-d2 and cAMP cryptate (Cisbio) for 1 hour at room temperature before fluorescence was measured with an EnVision® plate reader (PerkinElmer).

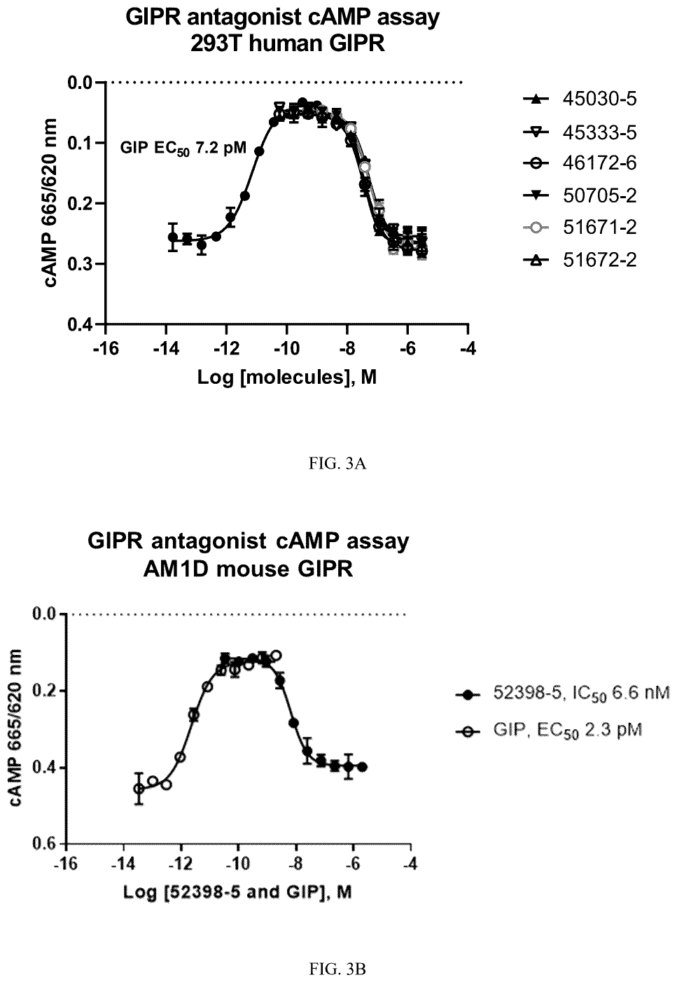

Representative cAMP levels for six conjugates (45030-5, 45333-5, 46172-6, 50705-2, 51671-2, and 51672-2) and a positive control (recombinant human GIP) are expressed as a fluorescence ratio of 665/620 nm and shown in FIG. 3A for the HEK 293T cells stably expressing the human GIPR. In addition, cAMP levels for conjugate 52398-5 and a positive control (recombinant mouse GIP) are shown in FIG. 3B, where each data point represents the mean of two biological replicates±standard deviation.

Additionally, IC₅₀ values for human GIPR determined using this functional assay with HEK 293T cells stably expressing the human GIPR are provided for certain example conjugates in Table 19.

### Example 4: KinExA® Analysis of Binding to Human GIPR Extracellular Domain (ECD)

Binding of the anti-GIPR antagonist/GCGR receptor agonist bispecific conjugate 51671 to human GIPR was measured by a solution equilibrium binding assay using KinExA® 3200 (Sapidyne Instruments Inc., Boise, ID). In brief, 100 mg (dry weight) bis-acrylamide/azlactone copolymer beads (Thermo Scientific (Pierce), Catalog No. 53110) was pre-coated with 50 pg human GIPR ECD in 50 mM Na₂CO₃, pH 9.6 at 4° C. overnight. The ligand protein coated beads were then blocked with 10 mg/mL BSA (Sigma-Aldrich, St. Louis, MO) in 1 M Tris-HCl, pH 8.0 at 4° C. for 2 hours. The beads were then re-suspended in 35 mL beads buffer containing PBS with 0.1 mg/mL BSA, 0.005% v/v P20, and 0.04% sodium azide. Prior to analysis, 30 pM, 100 pM, and 300 pM of conjugate were mixed with increasing concentrations (0.268 pM to 100 nM) of human GIPR ECD and equilibrated for over 8 hours at room temperature in sample buffer containing PBS with 0.1 mg/mL BSA and 0.005% v/v P20. The mixtures were then passed over the GIPR-coated beads. The amount of bead-bound conjugate was quantified using fluorescent Alexa Fluor® 647 labeled goat anti-huFc antibodies (Jackson Immuno Research, West Grove, PA) at 1 μg/mL in SuperBlock™ (Pierce Biotechnology, Inc., Rockford, IL). Since only free conjugate molecules in the mixtures can bind to huGIPR-coated beads, the quantified florescent binding signal is proportional to the concentration of free conjugate at equilibrium with a given GIPR concentration. The dissociation equilibrium constant (K_{D}) was obtained from global nonlinear regression of the “competition” curves using a n-curve one-site homogeneous binding model provided in the KinExA Pro software (Table 20).

TABLE 20
Equilibrium Dissociation Constants
for 51671 Binding to huGIPR ECD

95% Confidence Interval

| huGIPR ECD binding K_{D} [pM] | K_{D} High | K_{D} LOW |
|---|---|---|
| 106 | 134 | 82 |

### Section 3: In Vivo Characterization of GCGR Agonist Conjugate Constructs

This section provides in vivo characterization data for example GCGR agonist constructs. GCGR agonists conjugated to the anti-GIPR antagonist antibody aGIPR 5G12 SEFL2 E384C were used for murine studies as the anti-GIPR antagonist antibody aGIPR 2G10 SEFL2 E384C does not fully antagonize mouse GIPR at assay-relevant concentrations (Table 21). Molecules comprising aGIPR 5G12 SEFL2 E384C in place of aGIPR 2G10 SEFL2 E384C but the same GCGR agonist peptide and linker can be considered mouse surrogates of the aGIPR 2G10 SEFL2 E384C molecule.

TABLE 21
Comparisons of Mouse and Human In
Vitro GIPR Antagonist Activity

| | | hGIPR IC₅₀ | mGIPR |
|---|---|---|---|
| Identifier | Description | (nM) | IC₅₀ (nM) |
| PL-46818 | GIPR Ab 5G12 | 213.8 | 6.2 |
| PL-48946 | GIPR Ab 2G10 | 91.5 | 1176 |

Additionally, GCGR agonists conjugated to anti-DNP antibodies were used in certain experiments; the anti-DNP antibody acts as a half-life extending carrier immunoglobulin for its conjugated GCGR agonist and is not expected to contribute to biological activity beyond extension of GCGR agonist half-life.

### Example 5: Body Weight Changes in Diet-Induced Obese (DIO) Mice

Naïve male C57BL6 mice from Envigo or the Jackson Laboratory were fed a high-fat diet (D12492; Research Diets Inc.) for 14-16 weeks starting at 5-6 weeks of age. Mice were acclimated to all study-related procedures prior to study initiation and were then sorted into treatment groups with equal distribution of body weight (n=7 per group). Mice received intraperitoneal injection of either vehicle (10 mM sodium acetate, 9% sucrose, pH 5.2) or other test articles in 10 mM sodium acetate, 9% sucrose, pH 5.2 at each specified dose level on day 0. Body weight was monitored throughout the study.

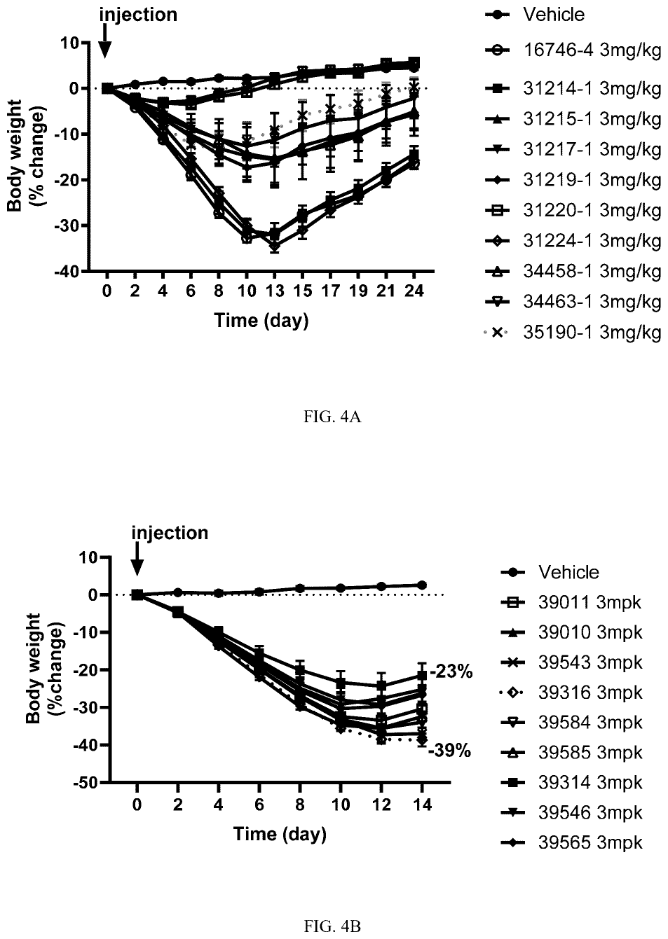

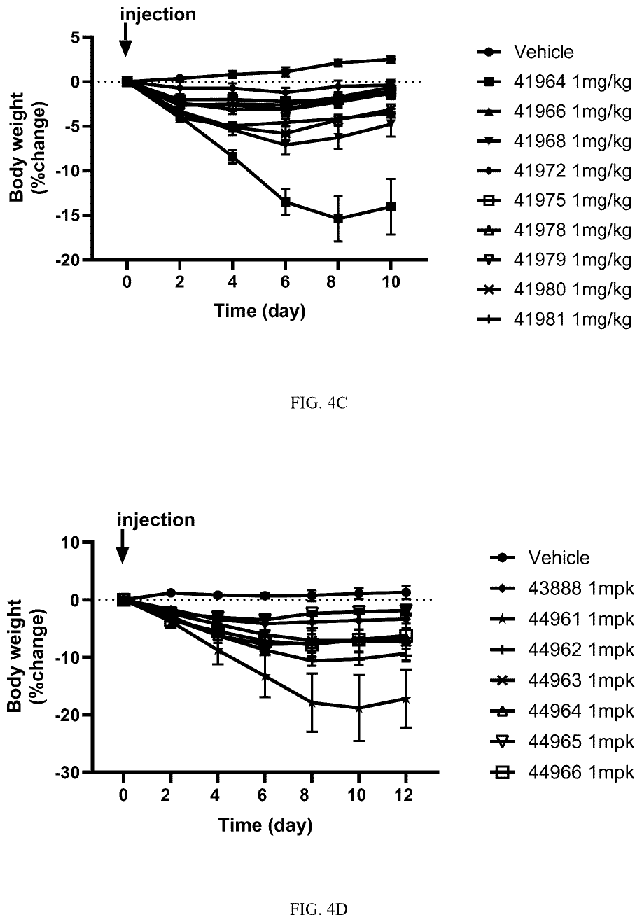

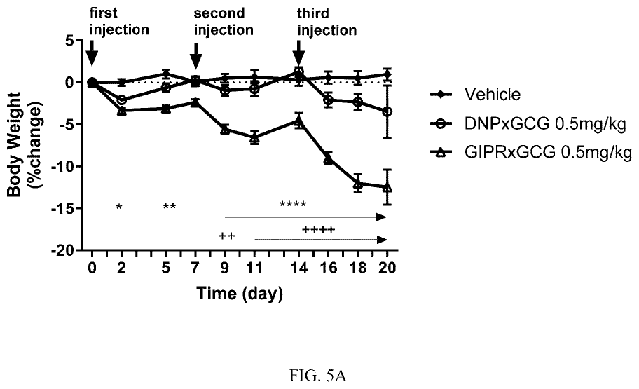

Changes in body weight from baseline are shown in FIG. 4A for ten example GCGR agonists conjugated to anti-DNP antibodies dosed at 3 mg/kg on day 0.

Changes in body weight from baseline are shown in FIG. 4B for ten example GCGR agonists conjugated to anti-GIPR antibodies dosed at 3 mg/kg on day 0.

Changes in body weight from baseline are shown in FIG. 4C for nine example GCGR agonists conjugated to anti-GIPR antibodies dosed at 1 mg/kg on day 0.

Changes in body weight from baseline are shown in FIG. 4D for seven example GCGR agonists conjugated to anti-GIPR antibodies dosed at 1 mg/kg on day 0.

In general, administration of the conjugates led to reduced body weight in mice with diet-induced obesity.

### Example 6: Body Weight Changes in DIO Mice for a Glucagon (GCG) Receptor Agonist Conjugated to an Anti-DNP Antibody (DNP×GCG) Vs. An Anti-GIPR Antibody (GIPR×GCG)

Naïve male C57BL6 mice from Envigo were fed a high-fat diet (D12492; Research Diets Inc.) for 15 weeks starting at 5 weeks of age. Mice were acclimated to all study-related procedures prior to study initiation and were then sorted into 3 treatment groups (n=7-8 per group) with equal distribution of body weight. Mice received intraperitoneal injections of either vehicle (10 mM sodium acetate, 9% sucrose, pH 5.2), DNP×GCG 0.5 mg/kg in 10 mM sodium acetate, 9% sucrose, pH 5.2, or GIPR×GCG 0.5 mg/kg in 10 mM sodium acetate, 9% sucrose, pH 5.2 on days 0, 7, and 14 at the same time in the morning.

DNP×GCG corresponds to conjugate 16746, while GIPR×GCG includes the same GCGR agonist peptide (SEQ ID NO: 1596) and linker (SEQ ID NO: 1629) conjugated to an aGIPR 5G12 SEFL2 E384C antibody.

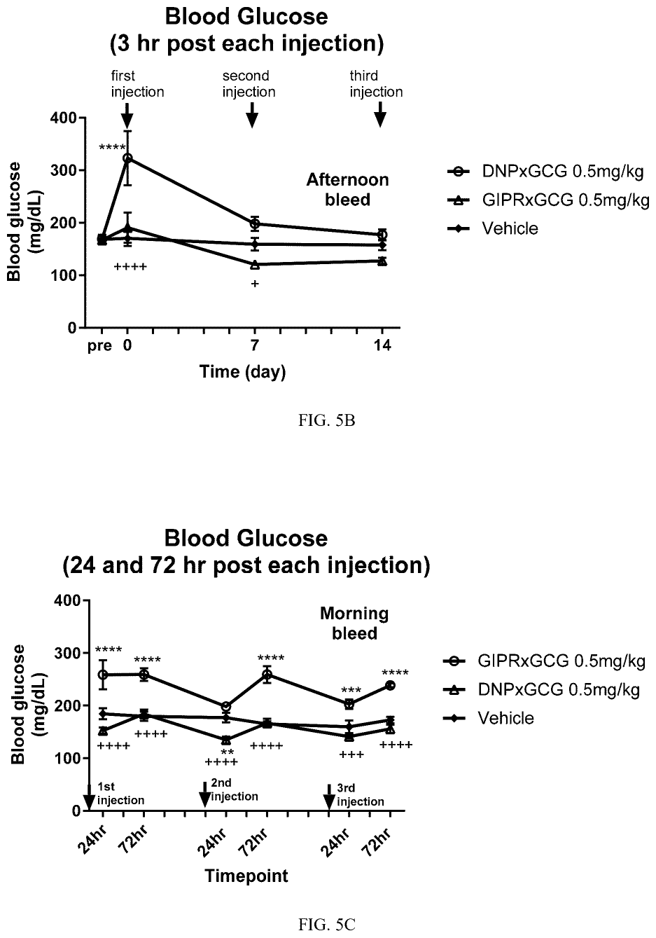

Three (3), 24, and 72 hours after each injection, conscious mice were retro-orbitally bled for glucose measurement (FIGS. 5B and 5C). Glucose was measured on handheld glucometer designed for rodent blood (AlphaTRAK®). Body weight was monitored throughout the study (FIG. 5A). The purpose of this study was not to maximize body weight reduction but to compare effects on body weight and glucose levels with anti-GIPR antibody and anti-DNP antibody when conjugated to the same GCG. Data were analyzed by two-way ANOVA using GraphPad Prism software and statistical significance is denoted as *p<0.05, **p<0.01, ***p<0.001 or ****p<0.0001 versus vehicle and +p<0.05, ++p<0.01, +++p<0.001 or ++++p<0.0001 GIPR×GCG versus DNP×GCG.

One of the primary functions of GIP is to stimulate insulin secretion from pancreatic R cells in a glucose-dependent manner (Dupre et al., J Clin Endocrinol Metab, 37(5), 826-828, 1973). Inhibition of GIPR may lead to a reduction in insulin secretion, which subsequently results in elevated blood glucose levels. Conversely, GCG has an established physiological role as a hormone that increases blood glucose by elevating hepatic glucose production via enhanced glycogen breakdown and stimulation of gluconeogenesis (Jiang & Zhang, Am J Physiol Endocrinol Metab, 284(4), E671-678, 2003). It counterbalances the glucose-lowering effects of insulin, thereby maintaining tight homeostatic control of blood glucose levels across a broad range of metabolic situations (Roder, Wu, Liu, & Han, Exp Mol Med, 48(3), e219, 2016). Consequently, the simultaneous inhibition of GIPR and stimulation of GCGR may lead to an increase in blood glucose levels, given their divergent functions in the regulation of glucose metabolism.

Unexpectedly, this study revealed that GIPR antagonism counteracted the GCG-induced glucose excursion in obese mice. In this study, the effects of vehicle control, long-acting GCG (DNP×GCG), and GIPR×GCG were compared in diet-induced obese (DIO) mice. The mice treated with GIPR×GCG showed more weight loss than the mice treated with long-acting GCG (FIG. 5A). As expected, the long-acting GCG-treated mice showed an increase in glucose levels when measured at 3 hrs after the first injection (FIG. 5B); however, the increase was not observed after the second or the third injection (FIG. 5C). Surprisingly, the transiently increased glucose levels were ameliorated in the mice treated with GIPR×GCG. Similar results were observed when glucose levels were measured at 24 hrs post each injection. These data suggest that when anti-GIPR antibody is utilized, the conjugate promotes more weight loss compared to GCG alone and counteracts GCG's effects on increasing glucose levels. Taken together, the data suggest that GIPR×GCG not only reduces body weight but also improves glucose homeostasis.

### Example 7: Body Weight, Liver Weight, Fat Mass, Fasting Plasma Glucose, Fasting Plasma Insulin, and Plasma Lipid Profile Changes in DIO Mice

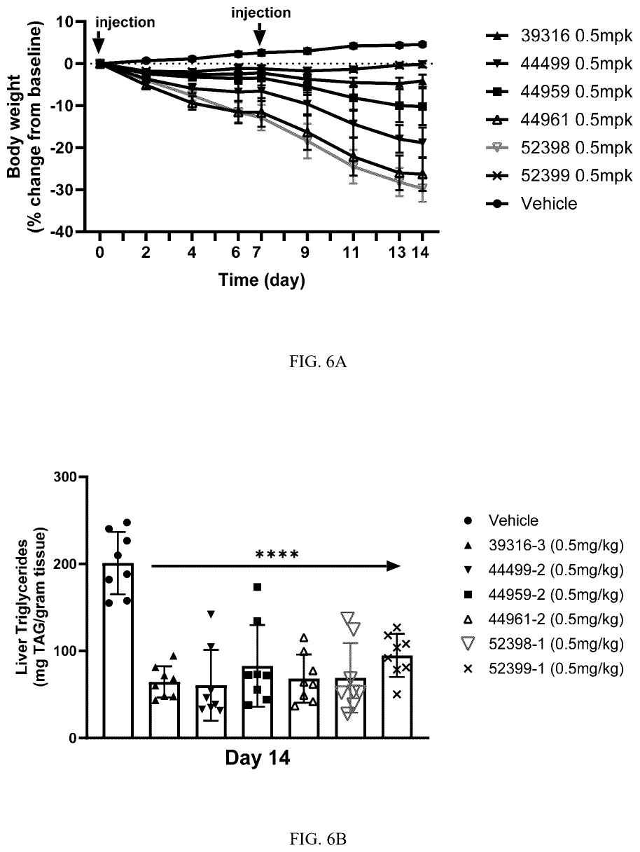

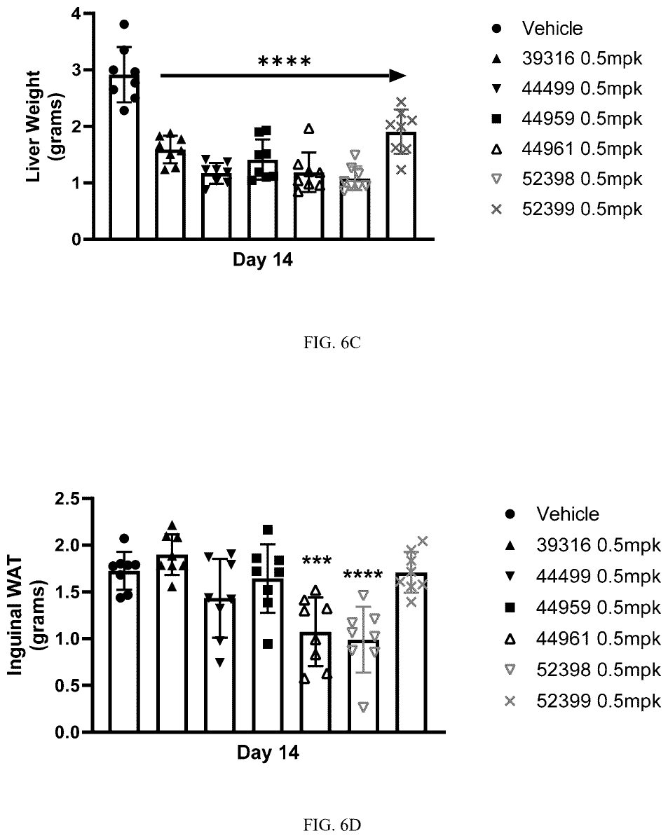

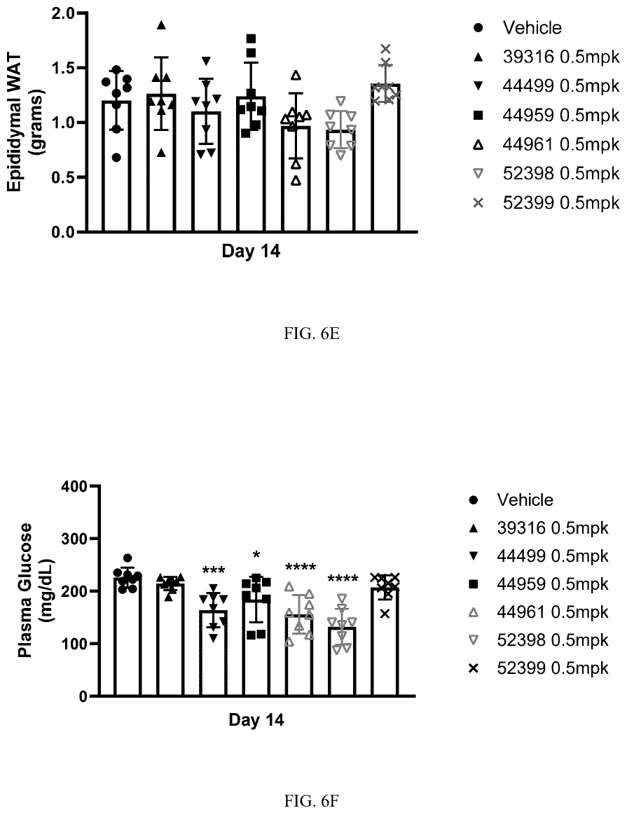

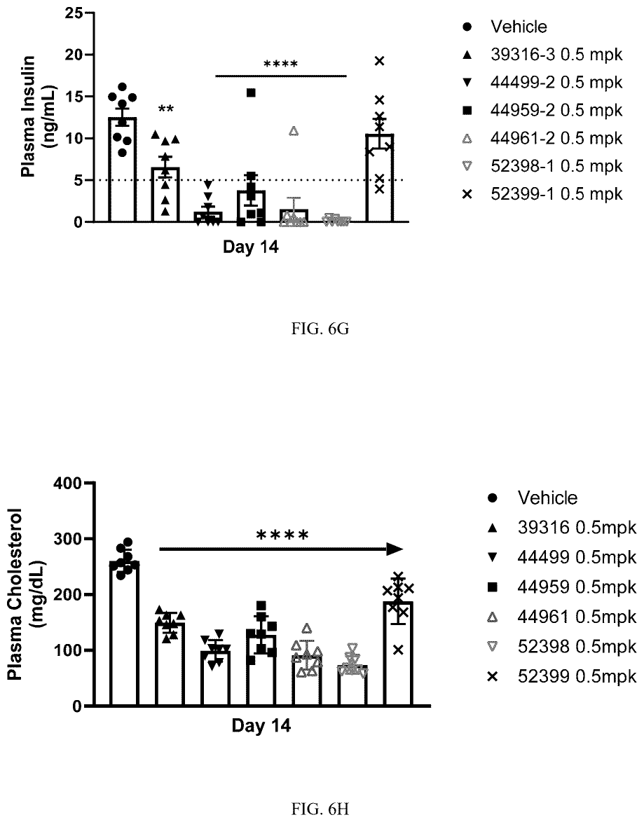

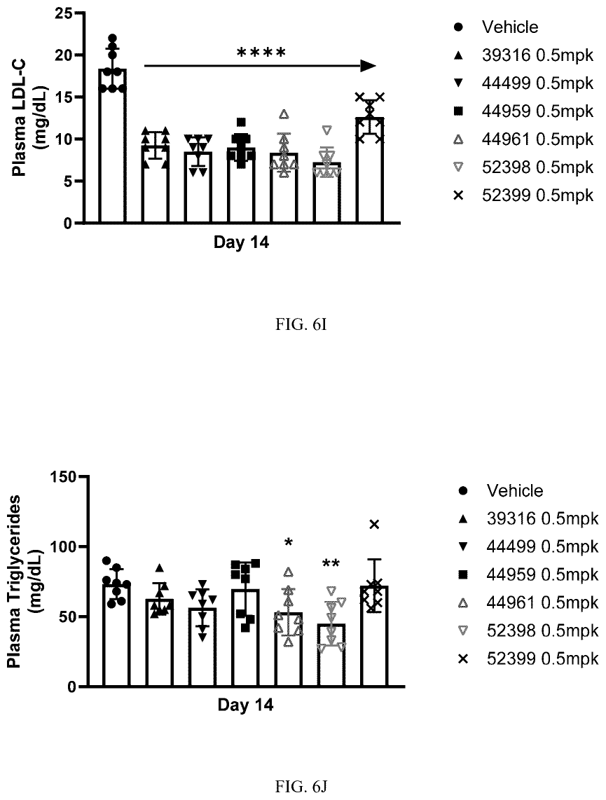

Naïve male C57BL6 mice from the Jackson Laboratory were fed a high-fat diet (D12492; Research Diets Inc.) for 14 weeks starting at 6 weeks of age. Mice were acclimated to all study-related procedures prior to study initiation and were then sorted into treatment groups with equal distribution of body weight. Mice received intraperitoneal injection of either vehicle (10 mM sodium acetate, 9% sucrose, pH 5.2) or a GIPR×GCG conjugate at 0.5 mg/kg in 10 mM sodium acetate, 9% sucrose, pH 5.2 on day 0 and day 7. Body weight was monitored throughout the study (FIG. 6A). A subcutaneous injection of lactated Ringer's solution was administered to mice as needed with robust weight loss and low food intake per veterinary clinical staff to prevent any dehydration concerns. All mice were bright, alert, and responsive throughout the study. Mice were euthanized after 4-hour fast on day 14 and trunk blood was collected into EDTA tubes for further plasma analysis. Tissue collection and tissue weights included inguinal and epidydimal white adipose tissue and liver.

Administration of GIPR×GCG conjugates reduced body weight (FIG. 6A) and liver triglycerides (FIG. 6B) in the diet-induced obese (DIO) mice. Additionally, administration of GIPR×GCG conjugates significantly reduced liver weight (FIG. 6C) and fat mass (FIGS. 6D, 6E) in the DIO mice. Moreover, administration of GIPR×GCG conjugates significantly reduced fasting plasma glucose (FIG. 6F) and insulin (FIG. 6G) levels and improved lipid levels (FIGS. 6H-6J) in the DIO mice.

Data in FIGS. 6B-6J are represented as group mean±standard error of mean (SE). Comparisons between treatment groups and vehicle were performed using a one-way ANOVA with Dunnett's multiple comparisons in GraphPad Prism (*p<0.05, ***p<0.001, ****p<0.0001 vs. vehicle).

### Example 8: Oral Glucose Tolerance Test (OGTT)

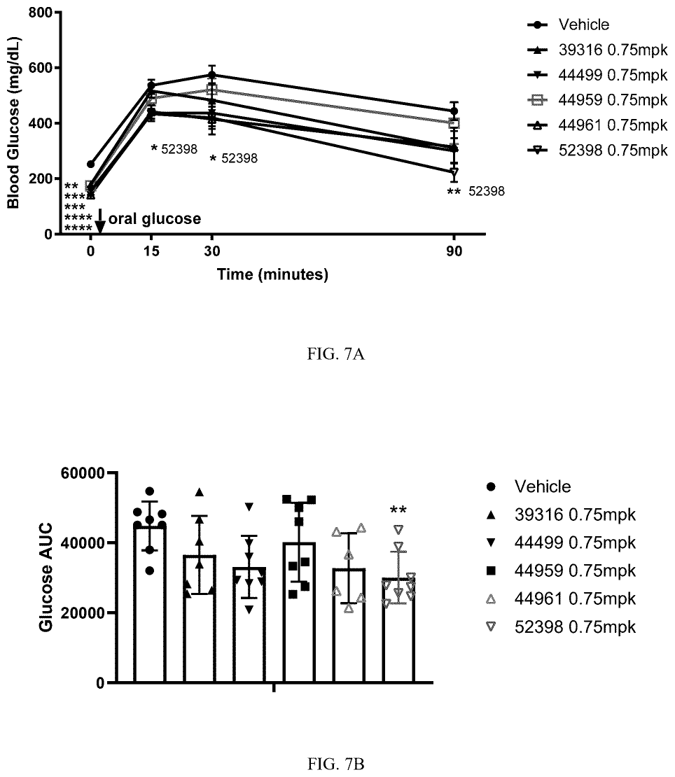

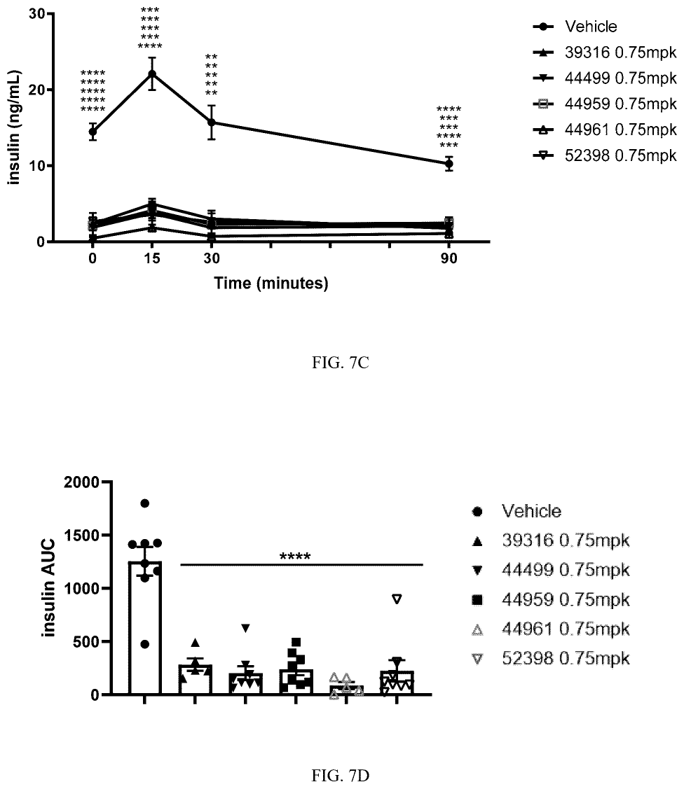

Naïve male C57B16 from the Jackson Laboratory were fed a high-fat diet (D12492; Research Diets Inc.) for 15 weeks starting at 6 weeks of age. Mice were acclimated to all study-related procedures prior to study initiation and were then sorted into 2 treatment groups (n=6-8 per group) with equal distribution of body weight. Mice received intraperitoneal injections of either vehicle (10 mM sodium acetate, 9% sucrose, pH 5.2) or various GIPR×GCG conjugates at 0.75 mg/kg in 10 mM sodium acetate, 9% sucrose, pH 5.2 on day 0, and body weight and food intake were measured on day 0, day 2, and day 4. On day 4, mice were fasted for 6 hours and then conscious mice were retro-orbitally bled for baseline glucose and insulin measurements. Mice then received an oral glucose bolus (1 g/kg) and were bled again 15, 30, and 90-minutes post oral glucose for glucose and insulin measurements. Glucose levels (FIGS. 7A, 7B) were measured on a handheld glucometer designed for rodent blood (AlphaTRAK®), and insulin levels (FIG. 7C, 7D) were measured using a commercial ELISA assay. Terminal blood sample was collected after the 90-minute timepoint for drug concentration measurements. The OGTT occurred 4 days (96 hours) post-treatment. Data were analyzed by two-way ANOVA using GraphPad Prism software and statistical significance is denoted as *p<0.05, **p<0.01, ***p<0.001, ****p<0.0001 versus vehicle. Data in FIGS. 7A-7D are represented as group mean±standard error of mean (SE).

In FIG. 7A, statistical significance is shown to the left of the first data points at time zero for 39316, 44499, 44959, 44961, and 52398 relative to vehicle in that order from top to bottom. At the 15, 30, and 90 minute timepoints, only 52398 was statistically significant relative to vehicle.

In FIG. 7C, statistical significance at each time point is plotted above the time point data in order (from top to bottom) of 39316, 44499, 44959, 44961, and 52398 relative to vehicle.

Improvement in glucose and insulin levels during OGTT was observed with 4-day pre-treatment with GIPR×GCG conjugates.

### Example 9: Combination Treatment with the GLP-1 Agonist Semaglutide

Naïve male C57B16 mice from the Jackson Laboratory were fed a high-fat diet (D12492; Research Diets Inc.) for 16 weeks starting at 6 weeks of age prior to study initiation. Prior to study start, mice were acclimated to all study-related procedures (handling, weighing, dosing, etc.) and were then randomized into four treatment groups with equal distribution of body weights across all groups.

The first treatment group received vehicle only. All mice in this treatment group were given a daily subcutaneous injection of PBS as vehicle at 2 mL/kg from day 0 to day 27. On days 18 and 24, all mice were also given an i.p. injection of 10 mM sodium acetate, 9% sucrose, pH 5.2 at 2.5 mL/kg.

The second treatment group received the GLP-1 agonist semaglutide only throughout the study period. All mice in this treatment group were given a daily subcutaneous injection of semaglutide from day 0 to day 27. The semaglutide dose on day 0 and day 1 was 0.004 mg/kg; from day 2 to day 27, the semaglutide dose was 0.012 mg/kg. On days 18 and 24, this group was also given an i.p. injection of 10 mM sodium acetate, 9% sucrose, pH 5.2 at 2.5 mL/kg.

The third treatment group received semaglutide for 17 days and then received a GIPR×GCG conjugate (52398) on days 18 and 24. Specifically, all mice in this treatment group were given a daily subcutaneous injection of semaglutide from day 0 to day 17. The semaglutide dose on day 0 and day 1 was 0.004 mg/kg; from day 2 to day 17, the semaglutide dose was 0.012 mg/kg. On days 18 and 24, mice were given an i.p. injection of 0.5 mg/kg of 52398.

The fourth treatment group received semaglutide for 27 days and also received a GIPR×GCG conjugate (52398) on days 18 and 24. Specifically, all mice in this treatment group were given a daily subcutaneous injection of semaglutide from day 0 to day 27. The semaglutide dose on day 0 and day 1 was 0.004 mg/kg; from day 2 to day 27, the semaglutide dose was 0.012 mg/kg. On day 18 and day 24, the mice were also given an i.p. injection of 52398. On day 18, mice received 0.5 mg/kg of 52398. On day 24, mice received a lower dose of 52398 (0.25 mg/kg) due to robust body weight reduction.

Body weight and food intake measurements were recorded throughout the study, and injections were given at approximately 8 AM each day. A subcutaneous injection of lactated Ringer's solution was administered to mice as needed with robust weight loss and low food intake per veterinary clinical staff to prevent any dehydration concerns. All mice were bright, alert, and responsive throughout the study. Mice were euthanized after 4-hour fast on day 28 and trunk blood was collected into EDTA tubes for further plasma analysis. Tissue collection and tissue weights included inguinal (ING) white adipose tissue (WAT) (subcutaneous), epidydimal (EPI) WAT (visceral), kidney (paired weights), liver, and brain.

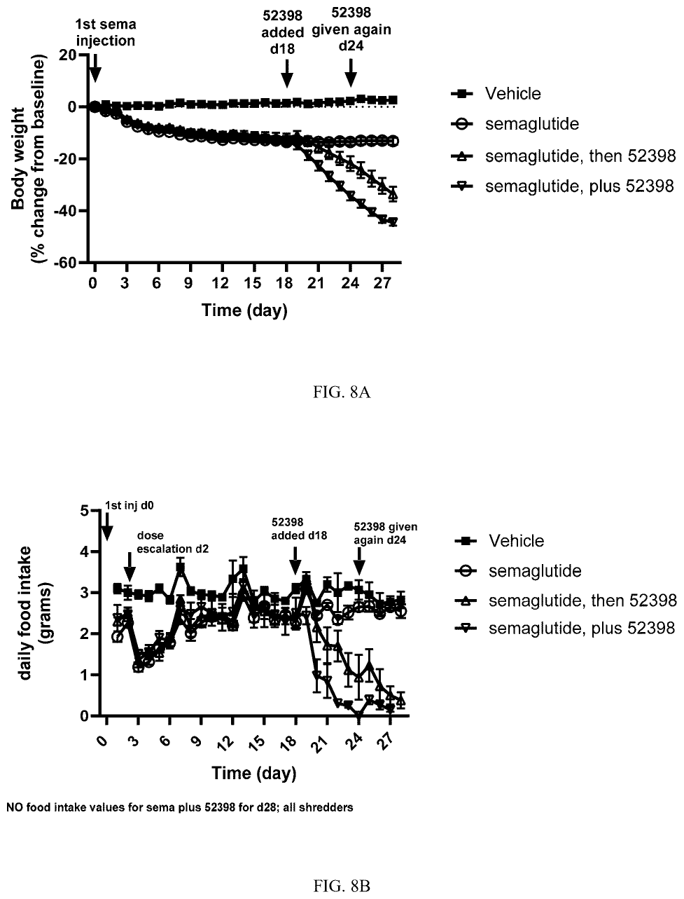

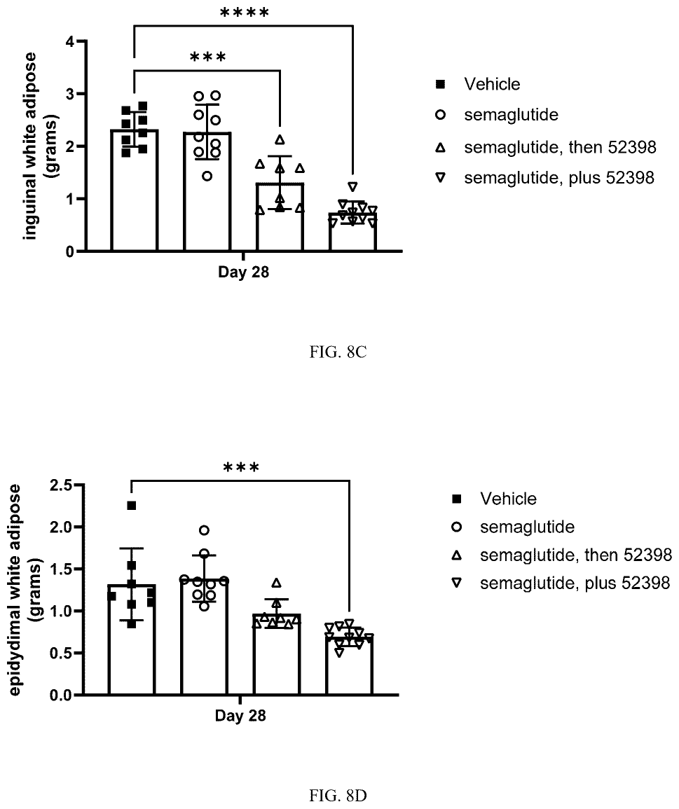

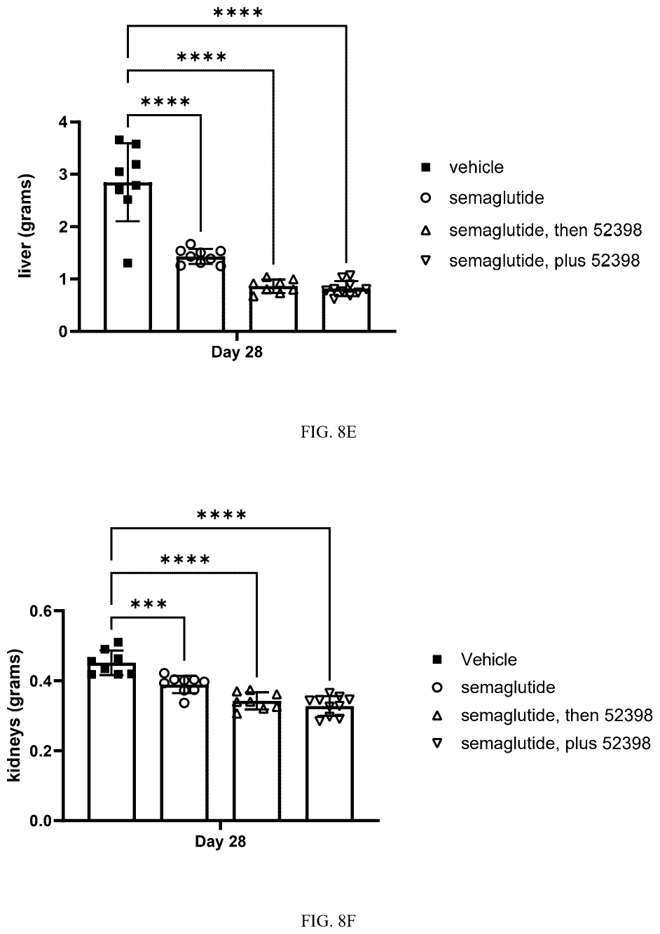

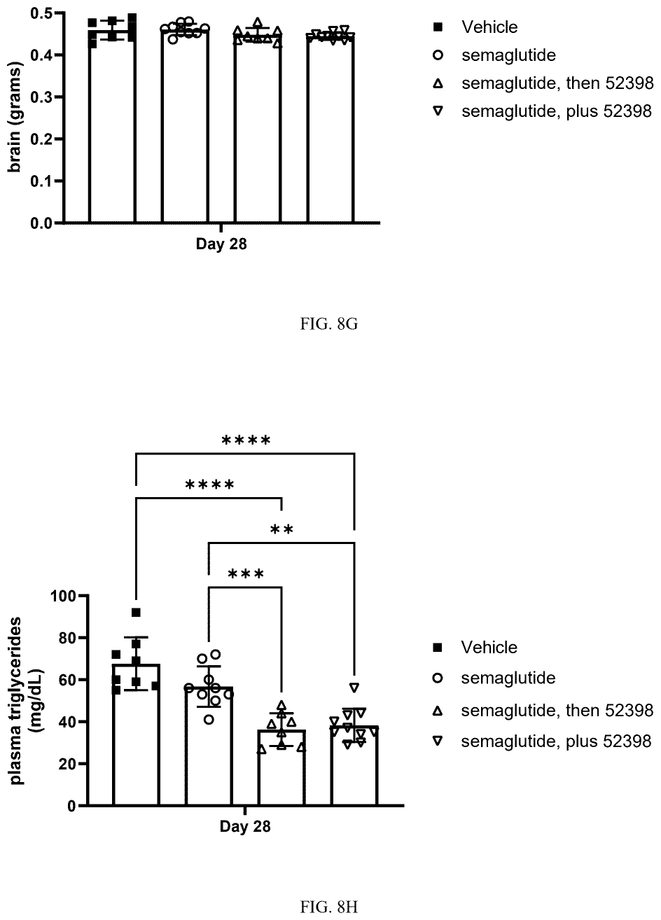

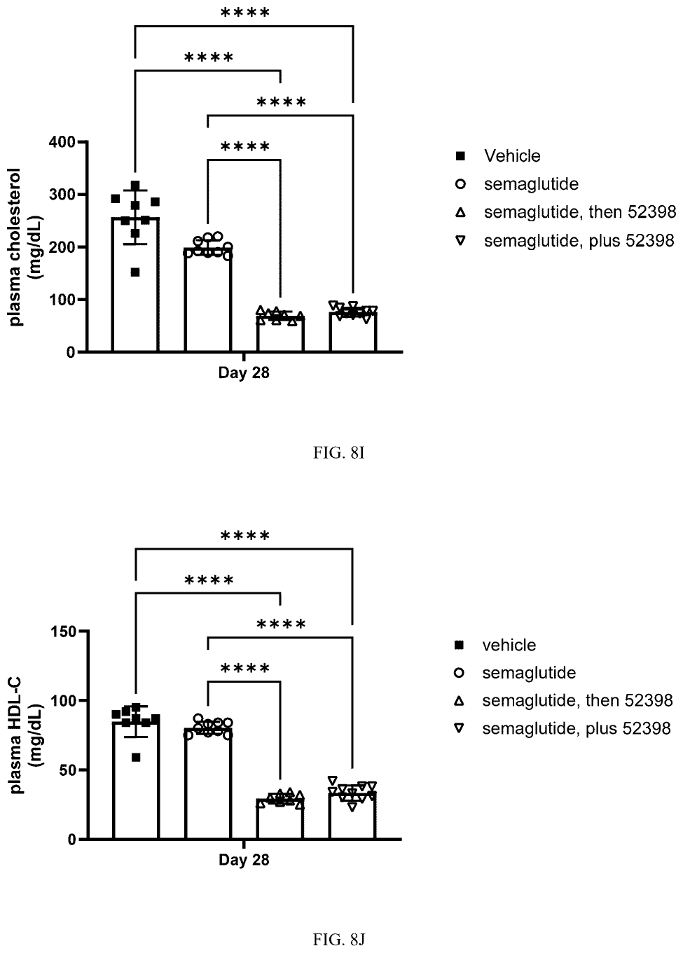

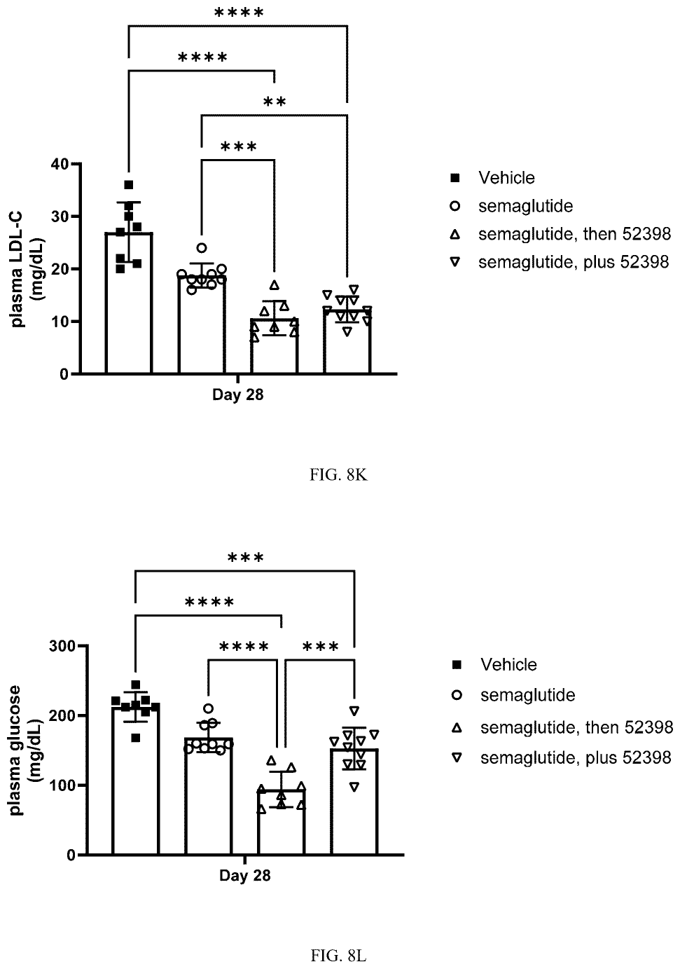

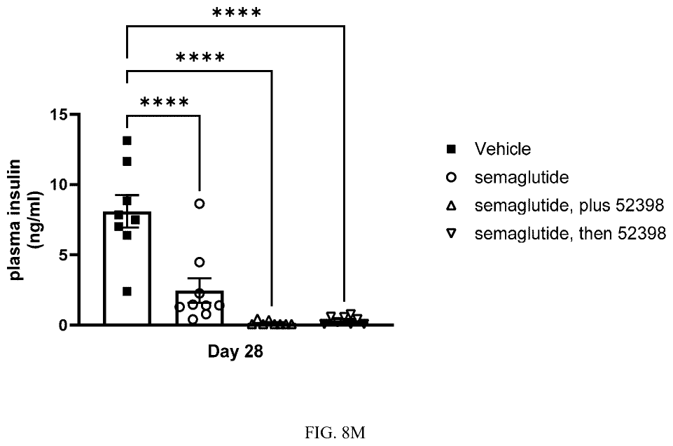

Combination therapy, with sequential or simultaneous semaglutide dosing, led to improved body weight reduction (FIG. 8A), reduced food intake (FIG. 8B), a trend toward reduced white adipose depots (FIGS. 8C, 8D), reduced liver (FIG. 8E) and kidney (FIG. 8F) weights without reduction in brain weight (FIG. 8G), improved lipid profiles (FIGS. 8H-8K), and improved plasma glucose (FIG. 8L) and insulin (FIG. 8M) levels in the DIO mice.

Data in FIGS. 8A-8M are represented as group mean±SE. Comparative statistics were not run for the data represented in FIGS. 8A and 8B. For FIG. 8B, no food intake values were collected for semaglutide in combination with 52398 on day 8 because all treated mice exhibited food shredding behavior in which mice shred high fat pellets and food intake could not be measured. Comparisons between treatment groups were performed for the data represented in FIGS. 8C-8M using a one-way ANOVA with Tukey's multiple comparisons test in GraphPad Prism (*p<0.05, **p<0.01, ***p<0.001, ****p<0.0001).

The section headings used herein are for organizational purposes only and are not to be construed as limiting the subject matter described.

All documents, or portions of documents, cited in this application, including but not limited to patents, patent applications, articles, books, and treatises, are hereby expressly incorporated by reference. What is described in an embodiment of the disclosure can be combined with one or more other embodiments of the disclosure unless context clearly indicates otherwise.

The disclosed subject matter is not intended to be limited in scope by the specific embodiments described herein, which are instead intended as non-limiting illustrations of individual aspects of the disclosure. Functionally equivalent methods and components are within the scope of the disclosure. Indeed, various modifications of the disclosed subject matter, in addition to those shown and described herein, will be apparent to those skilled in the art from the foregoing description and accompanying drawing(s). Such modifications are intended to fall within the scope of the disclosed subject matter.

The descriptions of the various embodiments and/or examples of the disclosed subject matter have been presented for purposes of illustration, but are not intended to be exhaustive or limiting in any way. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, and/or to enable others of ordinary skill in the art to understand the disclosed subject matter.

What is claimed is:

1. A molecule comprising:
a first polypeptide that agonizes a glucagon receptor (“GCGR”); and
an antibody that specifically binds to a glucose-dependent insulinotropic polypeptide receptor (“GIPR”).

2. The molecule of claim 1, wherein:
an ε-amino group of a lysine residue of the first polypeptide is covalently linked to a C-terminus of a first linker polypeptide; and
an N-terminus of the first linker polypeptide is conjugated to a cysteine residue of the antibody.

3. The molecule of claim 1, wherein:
a C-terminal amino acid residue of the first polypeptide is covalently linked to a N-terminal amino acid of a first linker polypeptide; and
a C-terminal amino acid residue of the first linker polypeptide is conjugated to a cysteine residue of the antibody.

4. The molecule of any one of claims 1-3, wherein the first polypeptide is glucagon or a glucagon analog.

5. The molecule of any one of claims 1-4, wherein the first polypeptide comprises the amino acid sequence of any one of SEQ ID NOs: 1587-1627, 1747-1840, 1859-1862, or 1879-1881.

6. The molecule of any one of claims 1-5, wherein the first polypeptide comprises the amino acid sequence of any one of SEQ ID NOs: 1587-1627 or 1747.

7. The molecule of any one of claims 1-5, wherein the first polypeptide comprises the amino acid sequence of any one of SEQ ID NOs: 1587, 1592, 1596, 1615, 1626, 1818, 1822, 1825, or 1826.

8. The molecule of any one of claims 1-5 or 7, wherein the first polypeptide comprises the amino acid sequence of any one of SEQ ID NOs: 1626, 1818, 1822, 1825, or 1826.

9. The molecule of any one of claims 1-8, wherein the first linker polypeptide comprises the amino acid sequence of any one of SEQ ID NOs: 1628-1683, 1739-1746, or 1841-1852.

10. The molecule of any one of claims 1-9, wherein the first linker polypeptide comprises the amino acid sequence of any one of SEQ ID NOs: 1628-1683.

11. The molecule of any one of claims 1-10, wherein the first linker polypeptide comprises the amino acid sequence of any one of SEQ ID NOs: 1628-1630.

12. The molecule of claim 2, wherein:
the first polypeptide comprises the amino acid sequence of any one of SEQ ID NOs: 1587-1627, 1747-1749, 1751-1792, 1794-1798, 1801-1830, 1833-1840, 1859-1862, or 1879-1881; and
the first polypeptide linker comprises the amino acid sequence of any one of SEQ ID NOs: 1628-1683, 1739, 1850, or 1851.

13. The molecule of claim 3, wherein:
the first polypeptide comprises the amino acid sequence of any one of SEQ ID NOs: 1793, 1799, 1800, 1831, or 1832; and
the first polypeptide linker comprises the amino acid sequence of any one of SEQ ID NOs: 1740-1746, 1841-1849, or 1852.

14. The molecule of any one of claims 2-12, wherein the cysteine residue of the antibody that is conjugated to the first linker polypeptide is at a position selected from the group consisting of 88 of a light chain, 384 of a heavy chain, and 487 of a heavy chain, according to AHo numbering.

15. The molecule of any one of claims 1-14, further comprising a second polypeptide that agonizes a GCGR.

16. The molecule of claim 15, wherein:
an ε-amino group of a lysine residue of the second polypeptide is covalently linked to a C-terminus of a second linker polypeptide; and
an N-terminus of the second linker polypeptide is conjugated to a cysteine residue of the antibody.

17. The molecule of claim 15, wherein:
a C-terminal amino acid residue of the second polypeptide is covalently linked to a N-terminal amino acid of a second linker polypeptide; and
a C-terminal amino acid residue of the second linker polypeptide is conjugated to a cysteine residue of the antibody.

18. The molecule of any one of claims 15-17, wherein the second polypeptide comprises the amino acid sequence of any one of SEQ ID NOs: 1587-1627, 1747-1840, 1859-1862, or 1879-1881.

19. The molecule of any one of claims 15-18, wherein the second polypeptide comprises the amino acid sequence of any one of SEQ ID NOs: 1587-1627 or 1747.

20. The molecule of any one of claims 15-18, wherein the second polypeptide comprises the amino acid sequence of any one of SEQ ID NOs: 1587, 1592, 1596, 1615, 1626, 1818, 1822, 1825, or 1826.

21. The molecule of any one of claims 15-18 or 20, wherein the second polypeptide comprises the amino acid sequence of any one of SEQ ID NOs: 1626, 1818, 1822, 1825, or 1826.

22. The molecule of any one of claims 15-21, wherein the first polypeptide has the same amino acid sequence as the second polypeptide.

23. The molecule of any one of claims 15-22, wherein the second linker polypeptide comprises the amino acid sequence of any one of SEQ ID NOs: 1628-1683, 1739-1746, or 1841-1852.

24. The molecule of any one of claims 15-23, wherein the second linker polypeptide comprises the amino acid sequence of any one of SEQ ID NOs: 1628-1683.

25. The molecule of any one of claims 15-24, wherein the second linker polypeptide comprises the amino acid sequence of any one of SEQ ID NOs: 1628-1630.

26. The molecule of any one of claims 15-25, wherein the first linker polypeptide has the same amino acid sequence as the second linker polypeptide.

27. The molecule of any one of claims 15-26, wherein the cysteine residue of the antibody that is conjugated to the second linker polypeptide is at a position selected from the group consisting of 88 of a light chain, 384 of a heavy chain, and 487 of a heavy chain, according to AHo numbering.

28. The molecule of any one of claims 1-27, wherein the antibody comprises a CDRL1, a CDRL2, a CDRL3, a CDRH1, a CDRH2, and a CDRH3, wherein the CDRL1, the CDRL2, the CDRL3, the CDRH1, the CDRH2, and the CDRH3 comprise amino acid sequences selected from:
i. SEQ ID NO: 629, SEQ ID NO: 786, SEQ ID NO: 943, SEQ ID NO: 1100, SEQ ID NO: 1257, and SEQ ID NO: 1414, respectively;
ii. SEQ ID NO: 630, SEQ ID NO: 787, SEQ ID NO: 944, SEQ ID NO: 1101, SEQ ID NO: 1258, and SEQ ID NO: 1415, respectively;
iii. SEQ ID NO: 631, SEQ ID NO: 788, SEQ ID NO: 945, SEQ ID NO: 1102, SEQ ID NO: 1259, and SEQ ID NO: 1416, respectively;
iv. SEQ ID NO: 632, SEQ ID NO: 789, SEQ ID NO: 946, SEQ ID NO: 1103, SEQ ID NO: 1260, and SEQ ID NO: 1417, respectively;
v. SEQ ID NO: 633, SEQ ID NO: 790, SEQ ID NO: 947, SEQ ID NO: 1104, SEQ ID NO: 1261, and SEQ ID NO: 1418, respectively;
vi. SEQ ID NO: 634, SEQ ID NO: 791, SEQ ID NO: 948, SEQ ID NO: 1105, SEQ ID NO: 1262, and SEQ ID NO: 1419, respectively;
vii. SEQ ID NO: 635, SEQ ID NO: 792, SEQ ID NO: 949, SEQ ID NO: 1106, SEQ ID NO: 1263, and SEQ ID NO: 1420, respectively;
viii. SEQ ID NO: 636, SEQ ID NO: 793, SEQ ID NO: 950, SEQ ID NO: 1107, SEQ ID NO: 1264, and SEQ ID NO: 1421, respectively;
ix. SEQ ID NO: 637, SEQ ID NO: 794, SEQ ID NO: 951, SEQ ID NO: 1108, SEQ ID NO: 1265, and SEQ ID NO: 1422, respectively;
x. SEQ ID NO: 638, SEQ ID NO: 795, SEQ ID NO: 952, SEQ ID NO: 1109, SEQ ID NO: 1266, and SEQ ID NO: 1423, respectively;
xi. SEQ ID NO: 639, SEQ ID NO: 796, SEQ ID NO: 953, SEQ ID NO: 1110, SEQ ID NO: 1267, and SEQ ID NO: 1424, respectively;
xii. SEQ ID NO: 640, SEQ ID NO: 797, SEQ ID NO: 954, SEQ ID NO: 1111, SEQ ID NO: 1268, and SEQ ID NO: 1425, respectively;
xiii. SEQ ID NO: 641, SEQ ID NO: 798, SEQ ID NO: 955, SEQ ID NO: 1112, SEQ ID NO: 1269, and SEQ ID NO: 1426, respectively;
xiv. SEQ ID NO: 642, SEQ ID NO: 799, SEQ ID NO: 956, SEQ ID NO: 1113, SEQ ID NO: 1270, and SEQ ID NO: 1427, respectively;
xv. SEQ ID NO: 643, SEQ ID NO: 800, SEQ ID NO: 957, SEQ ID NO: 1114, SEQ ID NO: 1271, and SEQ ID NO: 1428, respectively;
xvi. SEQ ID NO: 644, SEQ ID NO: 801, SEQ ID NO: 958, SEQ ID NO: 1115, SEQ ID NO: 1272, and SEQ ID NO: 1429, respectively;
xvii. SEQ ID NO: 645, SEQ ID NO: 802, SEQ ID NO: 959, SEQ ID NO: 1116, SEQ ID NO: 1273, and SEQ ID NO: 1430, respectively;
xviii. SEQ ID NO: 646, SEQ ID NO: 803, SEQ ID NO: 960, SEQ ID NO: 1117, SEQ ID NO: 1274, and SEQ ID NO: 1431, respectively;
xix. SEQ ID NO: 647, SEQ ID NO: 804, SEQ ID NO: 961, SEQ ID NO: 1118, SEQ ID NO: 1275, and SEQ ID NO: 1432, respectively;
xx. SEQ ID NO: 648, SEQ ID NO: 805, SEQ ID NO: 962, SEQ ID NO: 1119, SEQ ID NO: 1276, and SEQ ID NO: 1433, respectively;
xxi. SEQ ID NO: 649, SEQ ID NO: 806, SEQ ID NO: 963, SEQ ID NO: 1120, SEQ ID NO: 1277, and SEQ ID NO: 1434, respectively;
xxii. SEQ ID NO: 650, SEQ ID NO: 807, SEQ ID NO: 964, SEQ ID NO: 1121, SEQ ID NO: 1278, and SEQ ID NO: 1435, respectively;
xxiii. SEQ ID NO: 651, SEQ ID NO: 808, SEQ ID NO: 965, SEQ ID NO: 1122, SEQ ID NO: 1279, and SEQ ID NO: 1436, respectively;
xxiv. SEQ ID NO: 652, SEQ ID NO: 809, SEQ ID NO: 966, SEQ ID NO: 1123, SEQ ID NO: 1280, and SEQ ID NO: 1437, respectively;
xxv. SEQ ID NO: 653, SEQ ID NO: 810, SEQ ID NO: 967, SEQ ID NO: 1124, SEQ ID NO: 1281, and SEQ ID NO: 1438, respectively;
xxvi. SEQ ID NO: 654, SEQ ID NO: 811, SEQ ID NO: 968, SEQ ID NO: 1125, SEQ ID NO: 1282, and SEQ ID NO: 1439, respectively;
xxvii. SEQ ID NO: 655, SEQ ID NO: 812, SEQ ID NO: 969, SEQ ID NO: 1126, SEQ ID NO: 1283, and SEQ ID NO: 1440, respectively;
xxviii. SEQ ID NO: 656, SEQ ID NO: 813, SEQ ID NO: 970, SEQ ID NO: 1127, SEQ ID NO: 1284, and SEQ ID NO: 1441, respectively;
xxix. SEQ ID NO: 657, SEQ ID NO: 814, SEQ ID NO: 971, SEQ ID NO: 1128, SEQ ID NO: 1285, and SEQ ID NO: 1442, respectively;
xxx. SEQ ID NO: 658, SEQ ID NO: 815, SEQ ID NO: 972, SEQ ID NO: 1129, SEQ ID NO: 1286, and SEQ ID NO: 1443, respectively;
xxxi. SEQ ID NO: 659, SEQ ID NO: 816, SEQ ID NO: 973, SEQ ID NO: 1130, SEQ ID NO: 1287, and SEQ ID NO: 1444, respectively;
xxxii. SEQ ID NO: 660, SEQ ID NO: 817, SEQ ID NO: 974, SEQ ID NO: 1131, SEQ ID NO: 1288, and SEQ ID NO: 1445, respectively;
xxxiii. SEQ ID NO: 661, SEQ ID NO: 818, SEQ ID NO: 975, SEQ ID NO: 1132, SEQ ID NO: 1289, and SEQ ID NO: 1446, respectively;
xxxiv. SEQ ID NO: 662, SEQ ID NO: 819, SEQ ID NO: 976, SEQ ID NO: 1133, SEQ ID NO: 1290, and SEQ ID NO: 1447, respectively;
xxxv. SEQ ID NO: 663, SEQ ID NO: 820, SEQ ID NO: 977, SEQ ID NO: 1134, SEQ ID NO: 1291, and SEQ ID NO: 1448, respectively;
xxxvi. SEQ ID NO: 664, SEQ ID NO: 821, SEQ ID NO: 978, SEQ ID NO: 1135, SEQ ID NO: 1292, and SEQ ID NO: 1449, respectively;
xxxvii. SEQ ID NO: 665, SEQ ID NO: 822, SEQ ID NO: 979, SEQ ID NO: 1136, SEQ ID NO: 1293, and SEQ ID NO: 1450, respectively;
xxxviii. SEQ ID NO: 666, SEQ ID NO: 823, SEQ ID NO: 980, SEQ ID NO: 1137, SEQ ID NO: 1294, and SEQ ID NO: 1451, respectively;
xxxix. SEQ ID NO: 667, SEQ ID NO: 824, SEQ ID NO: 981, SEQ ID NO: 1138, SEQ ID NO: 1295, and SEQ ID NO: 1452, respectively;
xl. SEQ ID NO: 668, SEQ ID NO: 825, SEQ ID NO: 982, SEQ ID NO: 1139, SEQ ID NO: 1296, and SEQ ID NO: 1453, respectively;
xli. SEQ ID NO: 669, SEQ ID NO: 826, SEQ ID NO: 983, SEQ ID NO: 1140, SEQ ID NO: 1297, and SEQ ID NO: 1454, respectively;
xlii. SEQ ID NO: 670, SEQ ID NO: 827, SEQ ID NO: 984, SEQ ID NO: 1141, SEQ ID NO: 1298, and SEQ ID NO: 1455, respectively;
xliii. SEQ ID NO: 671, SEQ ID NO: 828, SEQ ID NO: 985, SEQ ID NO: 1142, SEQ ID NO: 1299, and SEQ ID NO: 1456, respectively;
xliv. SEQ ID NO: 672, SEQ ID NO: 829, SEQ ID NO: 986, SEQ ID NO: 1143, SEQ ID NO: 1300, and SEQ ID NO: 1457, respectively;
xlv. SEQ ID NO: 673, SEQ ID NO: 830, SEQ ID NO: 987, SEQ ID NO: 1144, SEQ ID NO: 1301, and SEQ ID NO: 1458, respectively;
xlvi. SEQ ID NO: 674, SEQ ID NO: 831, SEQ ID NO: 988, SEQ ID NO: 1145, SEQ ID NO: 1302, and SEQ ID NO: 1459, respectively;
xlvii. SEQ ID NO: 675, SEQ ID NO: 832, SEQ ID NO: 989, SEQ ID NO: 1146, SEQ ID NO: 1303, and SEQ ID NO: 1460, respectively;
xlviii. SEQ ID NO: 676, SEQ ID NO: 833, SEQ ID NO: 990, SEQ ID NO: 1147, SEQ ID NO: 1304, and SEQ ID NO: 1461, respectively;
xlix. SEQ ID NO: 677, SEQ ID NO: 834, SEQ ID NO: 991, SEQ ID NO: 1148, SEQ ID NO: 1305, and SEQ ID NO: 1462, respectively;
l. SEQ ID NO: 678, SEQ ID NO: 835, SEQ ID NO: 992, SEQ ID NO: 1149, SEQ ID NO: 1306, and SEQ ID NO: 1463, respectively;
li. SEQ ID NO: 679, SEQ ID NO: 836, SEQ ID NO: 993, SEQ ID NO: 1150, SEQ ID NO: 1307, and SEQ ID NO: 1464, respectively;
lii. SEQ ID NO: 680, SEQ ID NO: 837, SEQ ID NO: 994, SEQ ID NO: 1151, SEQ ID NO: 1308, and SEQ ID NO: 1465, respectively;
liii. SEQ ID NO: 681, SEQ ID NO: 838, SEQ ID NO: 995, SEQ ID NO: 1152, SEQ ID NO: 1309, and SEQ ID NO: 1466, respectively;
liv. SEQ ID NO: 682, SEQ ID NO: 839, SEQ ID NO: 996, SEQ ID NO: 1153, SEQ ID NO: 1310, and SEQ ID NO: 1467, respectively;
lv. SEQ ID NO: 683, SEQ ID NO: 840, SEQ ID NO: 997, SEQ ID NO: 1154, SEQ ID NO: 1311, and SEQ ID NO: 1468, respectively;
lvi. SEQ ID NO: 684, SEQ ID NO: 841, SEQ ID NO: 998, SEQ ID NO: 1155, SEQ ID NO: 1312, and SEQ ID NO: 1469, respectively;
lvii. SEQ ID NO: 685, SEQ ID NO: 842, SEQ ID NO: 999, SEQ ID NO: 1156, SEQ ID NO: 1313, and SEQ ID NO: 1470, respectively;
lviii. SEQ ID NO: 686, SEQ ID NO: 843, SEQ ID NO: 1000, SEQ ID NO: 1157, SEQ ID NO: 1314, and SEQ ID NO: 1471, respectively;
lix. SEQ ID NO: 687, SEQ ID NO: 844, SEQ ID NO: 1001, SEQ ID NO: 1158, SEQ ID NO: 1315, and SEQ ID NO: 1472, respectively;
lx. SEQ ID NO: 688, SEQ ID NO: 845, SEQ ID NO: 1002, SEQ ID NO: 1159, SEQ ID NO: 1316, and SEQ ID NO: 1473, respectively;
lxi. SEQ ID NO: 689, SEQ ID NO: 846, SEQ ID NO: 1003, SEQ ID NO: 1160, SEQ ID NO: 1317, and SEQ ID NO: 1474, respectively;
lxii. SEQ ID NO: 690, SEQ ID NO: 847, SEQ ID NO: 1004, SEQ ID NO: 1161, SEQ ID NO: 1318, and SEQ ID NO: 1475, respectively;
lxiii. SEQ ID NO: 691, SEQ ID NO: 848, SEQ ID NO: 1005, SEQ ID NO: 1162, SEQ ID NO: 1319, and SEQ ID NO: 1476, respectively;
lxiv. SEQ ID NO: 692, SEQ ID NO: 849, SEQ ID NO: 1006, SEQ ID NO: 1163, SEQ ID NO: 1320, and SEQ ID NO: 1477, respectively;
lxv. SEQ ID NO: 693, SEQ ID NO: 850, SEQ ID NO: 1007, SEQ ID NO: 1164, SEQ ID NO: 1321, and SEQ ID NO: 1478, respectively;
lxvi. SEQ ID NO: 694, SEQ ID NO: 851, SEQ ID NO: 1008, SEQ ID NO: 1165, SEQ ID NO: 1322, and SEQ ID NO: 1479, respectively;
lxvii. SEQ ID NO: 695, SEQ ID NO: 852, SEQ ID NO: 1009, SEQ ID NO: 1166, SEQ ID NO: 1323, and SEQ ID NO: 1480, respectively;
lxviii. SEQ ID NO: 696, SEQ ID NO: 853, SEQ ID NO: 1010, SEQ ID NO: 1167, SEQ ID NO: 1324, and SEQ ID NO: 1481, respectively;
lxix. SEQ ID NO: 697, SEQ ID NO: 854, SEQ ID NO: 1011, SEQ ID NO: 1168, SEQ ID NO: 1325, and SEQ ID NO: 1482, respectively;
lxx. SEQ ID NO: 698, SEQ ID NO: 855, SEQ ID NO: 1012, SEQ ID NO: 1169, SEQ ID NO: 1326, and SEQ ID NO: 1483, respectively;
lxxi. SEQ ID NO: 699, SEQ ID NO: 856, SEQ ID NO: 1013, SEQ ID NO: 1170, SEQ ID NO: 1327, and SEQ ID NO: 1484, respectively;
lxxii. SEQ ID NO: 700, SEQ ID NO: 857, SEQ ID NO: 1014, SEQ ID NO: 1171, SEQ ID NO: 1328, and SEQ ID NO: 1485, respectively;
lxxiii. SEQ ID NO: 701, SEQ ID NO: 858, SEQ ID NO: 1015, SEQ ID NO: 1172, SEQ ID NO: 1329, and SEQ ID NO: 1486, respectively;
lxxiv. SEQ ID NO: 702, SEQ ID NO: 859, SEQ ID NO: 1016, SEQ ID NO: 1173, SEQ ID NO: 1330, and SEQ ID NO: 1487, respectively;
lxxv. SEQ ID NO: 703, SEQ ID NO: 860, SEQ ID NO: 1017, SEQ ID NO: 1174, SEQ ID NO: 1331, and SEQ ID NO: 1488, respectively;
lxxvi. SEQ ID NO: 704, SEQ ID NO: 861, SEQ ID NO: 1018, SEQ ID NO: 1175, SEQ ID NO: 1332, and SEQ ID NO: 1489, respectively;
lxxvii. SEQ ID NO: 705, SEQ ID NO: 862, SEQ ID NO: 1019, SEQ ID NO: 1176, SEQ ID NO: 1333, and SEQ ID NO: 1490, respectively;
lxxviii. SEQ ID NO: 706, SEQ ID NO: 863, SEQ ID NO: 1020, SEQ ID NO: 1177, SEQ ID NO: 1334, and SEQ ID NO: 1491, respectively;
lxxix. SEQ ID NO: 707, SEQ ID NO: 864, SEQ ID NO: 1021, SEQ ID NO: 1178, SEQ ID NO: 1335, and SEQ ID NO: 1492, respectively;
lxxx. SEQ ID NO: 708, SEQ ID NO: 865, SEQ ID NO: 1022, SEQ ID NO: 1179, SEQ ID NO: 1336, and SEQ ID NO: 1493, respectively;
lxxxi. SEQ ID NO: 709, SEQ ID NO: 866, SEQ ID NO: 1023, SEQ ID NO: 1180, SEQ ID NO: 1337, and SEQ ID NO: 1494, respectively;
lxxxii. SEQ ID NO: 710, SEQ ID NO: 867, SEQ ID NO: 1024, SEQ ID NO: 1181, SEQ ID NO: 1338, and SEQ ID NO: 1495, respectively;
lxxxiii. SEQ ID NO: 711, SEQ ID NO: 868, SEQ ID NO: 1025, SEQ ID NO: 1182, SEQ ID NO: 1339, and SEQ ID NO: 1496, respectively;
lxxxiv. SEQ ID NO: 712, SEQ ID NO: 869, SEQ ID NO: 1026, SEQ ID NO: 1183, SEQ ID NO: 1340, and SEQ ID NO: 1497, respectively;
lxxxv. SEQ ID NO: 713, SEQ ID NO: 870, SEQ ID NO: 1027, SEQ ID NO: 1184, SEQ ID NO: 1341, and SEQ ID NO: 1498, respectively;
lxxxvi. SEQ ID NO: 714, SEQ ID NO: 871, SEQ ID NO: 1028, SEQ ID NO: 1185, SEQ ID NO: 1342, and SEQ ID NO: 1499, respectively;
lxxxvii. SEQ ID NO: 715, SEQ ID NO: 872, SEQ ID NO: 1029, SEQ ID NO: 1186, SEQ ID NO: 1343, and SEQ ID NO: 1500, respectively;
lxxxviii. SEQ ID NO: 716, SEQ ID NO: 873, SEQ ID NO: 1030, SEQ ID NO: 1187, SEQ ID NO: 1344, and SEQ ID NO: 1501, respectively;
lxxxix. SEQ ID NO: 717, SEQ ID NO: 874, SEQ ID NO: 1031, SEQ ID NO: 1188, SEQ ID NO: 1345, and SEQ ID NO: 1502, respectively;
xc. SEQ ID NO: 718, SEQ ID NO: 875, SEQ ID NO: 1032, SEQ ID NO: 1189, SEQ ID NO: 1346, and SEQ ID NO: 1503, respectively;
xci. SEQ ID NO: 719, SEQ ID NO: 876, SEQ ID NO: 1033, SEQ ID NO: 1190, SEQ ID NO: 1347, and SEQ ID NO: 1504, respectively;
xcii. SEQ ID NO: 720, SEQ ID NO: 877, SEQ ID NO: 1034, SEQ ID NO: 1191, SEQ ID NO: 1348, and SEQ ID NO: 1505, respectively;
xciii. SEQ ID NO: 721, SEQ ID NO: 878, SEQ ID NO: 1035, SEQ ID NO: 1192, SEQ ID NO: 1349, and SEQ ID NO: 1506, respectively;
xciv. SEQ ID NO: 722, SEQ ID NO: 879, SEQ ID NO: 1036, SEQ ID NO: 1193, SEQ ID NO: 1350, and SEQ ID NO: 1507, respectively;
xcv. SEQ ID NO: 723, SEQ ID NO: 880, SEQ ID NO: 1037, SEQ ID NO: 1194, SEQ ID NO: 1351, and SEQ ID NO: 1508, respectively;
xcvi. SEQ ID NO: 724, SEQ ID NO: 881, SEQ ID NO: 1038, SEQ ID NO: 1195, SEQ ID NO: 1352, and SEQ ID NO: 1509, respectively;
xcvii. SEQ ID NO: 725, SEQ ID NO: 882, SEQ ID NO: 1039, SEQ ID NO: 1196, SEQ ID NO: 1353, and SEQ ID NO: 1510, respectively;
xcviii. SEQ ID NO: 726, SEQ ID NO: 883, SEQ ID NO: 1040, SEQ ID NO: 1197, SEQ ID NO: 1354, and SEQ ID NO: 1511, respectively;
xcix. SEQ ID NO: 727, SEQ ID NO: 884, SEQ ID NO: 1041, SEQ ID NO: 1198, SEQ ID NO: 1355, and SEQ ID NO: 1512, respectively;
c. SEQ ID NO: 728, SEQ ID NO: 885, SEQ ID NO: 1042, SEQ ID NO: 1199, SEQ ID NO: 1356, and SEQ ID NO: 1513, respectively;
ci. SEQ ID NO: 729, SEQ ID NO: 886, SEQ ID NO: 1043, SEQ ID NO: 1200, SEQ ID NO: 1357, and SEQ ID NO: 1514, respectively;
cii. SEQ ID NO: 730, SEQ ID NO: 887, SEQ ID NO: 1044, SEQ ID NO: 1201, SEQ ID NO: 1358, and SEQ ID NO: 1515, respectively;
ciii. SEQ ID NO: 731, SEQ ID NO: 888, SEQ ID NO: 1045, SEQ ID NO: 1202, SEQ ID NO: 1359, and SEQ ID NO: 1516, respectively;
civ. SEQ ID NO: 732, SEQ ID NO: 889, SEQ ID NO: 1046, SEQ ID NO: 1203, SEQ ID NO: 1360, and SEQ ID NO: 1517, respectively;
cv. SEQ ID NO: 733, SEQ ID NO: 890, SEQ ID NO: 1047, SEQ ID NO: 1204, SEQ ID NO: 1361, and SEQ ID NO: 1518, respectively;
cvi. SEQ ID NO: 734, SEQ ID NO: 891, SEQ ID NO: 1048, SEQ ID NO: 1205, SEQ ID NO: 1362, and SEQ ID NO: 1519, respectively;
cvii. SEQ ID NO: 735, SEQ ID NO: 892, SEQ ID NO: 1049, SEQ ID NO: 1206, SEQ ID NO: 1363, and SEQ ID NO: 1520, respectively;
cviii. SEQ ID NO: 736, SEQ ID NO: 893, SEQ ID NO: 1050, SEQ ID NO: 1207, SEQ ID NO: 1364, and SEQ ID NO: 1521, respectively;
cix. SEQ ID NO: 737, SEQ ID NO: 894, SEQ ID NO: 1051, SEQ ID NO: 1208, SEQ ID NO: 1365, and SEQ ID NO: 1522, respectively;
cx. SEQ ID NO: 738, SEQ ID NO: 895, SEQ ID NO: 1052, SEQ ID NO: 1209, SEQ ID NO: 1366, and SEQ ID NO: 1523, respectively;
cxi. SEQ ID NO: 739, SEQ ID NO: 896, SEQ ID NO: 1053, SEQ ID NO: 1210, SEQ ID NO: 1367, and SEQ ID NO: 1524, respectively;
cxii. SEQ ID NO: 740, SEQ ID NO: 897, SEQ ID NO: 1054, SEQ ID NO: 1211, SEQ ID NO: 1368, and SEQ ID NO: 1525, respectively;
cxiii. SEQ ID NO: 741, SEQ ID NO: 898, SEQ ID NO: 1055, SEQ ID NO: 1212, SEQ ID NO: 1369, and SEQ ID NO: 1526, respectively;
cxiv. SEQ ID NO: 742, SEQ ID NO: 899, SEQ ID NO: 1056, SEQ ID NO: 1213, SEQ ID NO: 1370, and SEQ ID NO: 1527, respectively;
cxv. SEQ ID NO: 743, SEQ ID NO: 900, SEQ ID NO: 1057, SEQ ID NO: 1214, SEQ ID NO: 1371, and SEQ ID NO: 1528, respectively;
cxvi. SEQ ID NO: 744, SEQ ID NO: 901, SEQ ID NO: 1058, SEQ ID NO: 1215, SEQ ID NO: 1372, and SEQ ID NO: 1529, respectively;
cxvii. SEQ ID NO: 745, SEQ ID NO: 902, SEQ ID NO: 1059, SEQ ID NO: 1216, SEQ ID NO: 1373, and SEQ ID NO: 1530, respectively;
cxviii. SEQ ID NO: 746, SEQ ID NO: 903, SEQ ID NO: 1060, SEQ ID NO: 1217, SEQ ID NO: 1374, and SEQ ID NO: 1531, respectively;
cxix. SEQ ID NO: 747, SEQ ID NO: 904, SEQ ID NO: 1061, SEQ ID NO: 1218, SEQ ID NO: 1375, and SEQ ID NO: 1532, respectively;
cxx. SEQ ID NO: 748, SEQ ID NO: 905, SEQ ID NO: 1062, SEQ ID NO: 1219, SEQ ID NO: 1376, and SEQ ID NO: 1533, respectively;
cxxi. SEQ ID NO: 749, SEQ ID NO: 906, SEQ ID NO: 1063, SEQ ID NO: 1220, SEQ ID NO: 1377, and SEQ ID NO: 1534, respectively;
cxxii. SEQ ID NO: 750, SEQ ID NO: 907, SEQ ID NO: 1064, SEQ ID NO: 1221, SEQ ID NO: 1378, and SEQ ID NO: 1535, respectively;
cxxiii. SEQ ID NO: 751, SEQ ID NO: 908, SEQ ID NO: 1065, SEQ ID NO: 1222, SEQ ID NO: 1379, and SEQ ID NO: 1536, respectively;
cxxiv. SEQ ID NO: 752, SEQ ID NO: 909, SEQ ID NO: 1066, SEQ ID NO: 1223, SEQ ID NO: 1380, and SEQ ID NO: 1537, respectively;
cxxv. SEQ ID NO: 753, SEQ ID NO: 910, SEQ ID NO: 1067, SEQ ID NO: 1224, SEQ ID NO: 1381, and SEQ ID NO: 1538, respectively;
cxxvi. SEQ ID NO: 754, SEQ ID NO: 911, SEQ ID NO: 1068, SEQ ID NO: 1225, SEQ ID NO: 1382, and SEQ ID NO: 1539, respectively;
cxxvii. SEQ ID NO: 755, SEQ ID NO: 912, SEQ ID NO: 1069, SEQ ID NO: 1226, SEQ ID NO: 1383, and SEQ ID NO: 1540, respectively;
cxxviii. SEQ ID NO: 756, SEQ ID NO: 913, SEQ ID NO: 1070, SEQ ID NO: 1227, SEQ ID NO: 1384, and SEQ ID NO: 1541, respectively;
cxxix. SEQ ID NO: 757, SEQ ID NO: 914, SEQ ID NO: 1071, SEQ ID NO: 1228, SEQ ID NO: 1385, and SEQ ID NO: 1542, respectively;
cxxx. SEQ ID NO: 758, SEQ ID NO: 915, SEQ ID NO: 1072, SEQ ID NO: 1229, SEQ ID NO: 1386, and SEQ ID NO: 1543, respectively;
cxxxi. SEQ ID NO: 759, SEQ ID NO: 916, SEQ ID NO: 1073, SEQ ID NO: 1230, SEQ ID NO: 1387, and SEQ ID NO: 1544, respectively;
cxxxii. SEQ ID NO: 760, SEQ ID NO: 917, SEQ ID NO: 1074, SEQ ID NO: 1231, SEQ ID NO: 1388, and SEQ ID NO: 1545, respectively;
cxxxiii. SEQ ID NO: 761, SEQ ID NO: 918, SEQ ID NO: 1075, SEQ ID NO: 1232, SEQ ID NO: 1389, and SEQ ID NO: 1546, respectively;
cxxxiv. SEQ ID NO: 762, SEQ ID NO: 919, SEQ ID NO: 1076, SEQ ID NO: 1233, SEQ ID NO: 1390, and SEQ ID NO: 1547, respectively;
cxxxv. SEQ ID NO: 763, SEQ ID NO: 920, SEQ ID NO: 1077, SEQ ID NO: 1234, SEQ ID NO: 1391, and SEQ ID NO: 1548, respectively;
cxxxvi. SEQ ID NO: 764, SEQ ID NO: 921, SEQ ID NO: 1078, SEQ ID NO: 1235, SEQ ID NO: 1392, and SEQ ID NO: 1549, respectively;
cxxxvii. SEQ ID NO: 765, SEQ ID NO: 922, SEQ ID NO: 1079, SEQ ID NO: 1236, SEQ ID NO: 1393, and SEQ ID NO: 1550, respectively;
cxxxviii. SEQ ID NO: 766, SEQ ID NO: 923, SEQ ID NO: 1080, SEQ ID NO: 1237, SEQ ID NO: 1394, and SEQ ID NO: 1551, respectively;
cxxxix. SEQ ID NO: 767, SEQ ID NO: 924, SEQ ID NO: 1081, SEQ ID NO: 1238, SEQ ID NO: 1395, and SEQ ID NO: 1552, respectively;
cxl. SEQ ID NO: 768, SEQ ID NO: 925, SEQ ID NO: 1082, SEQ ID NO: 1239, SEQ ID NO: 1396, and SEQ ID NO: 1553, respectively;
cxli. SEQ ID NO: 769, SEQ ID NO: 926, SEQ ID NO: 1083, SEQ ID NO: 1240, SEQ ID NO: 1397, and SEQ ID NO: 1554, respectively;
cxlii. SEQ ID NO: 770, SEQ ID NO: 927, SEQ ID NO: 1084, SEQ ID NO: 1241, SEQ ID NO: 1398, and SEQ ID NO: 1555, respectively;
cxliii. SEQ ID NO: 771, SEQ ID NO: 928, SEQ ID NO: 1085, SEQ ID NO: 1242, SEQ ID NO: 1399, and SEQ ID NO: 1556, respectively;
cxliv. SEQ ID NO: 772, SEQ ID NO: 929, SEQ ID NO: 1086, SEQ ID NO: 1243, SEQ ID NO: 1400, and SEQ ID NO: 1557, respectively;
cxlv. SEQ ID NO: 773, SEQ ID NO: 930, SEQ ID NO: 1087, SEQ ID NO: 1244, SEQ ID NO: 1401, and SEQ ID NO: 1558, respectively;
cxlvi. SEQ ID NO: 774, SEQ ID NO: 931, SEQ ID NO: 1088, SEQ ID NO: 1245, SEQ ID NO: 1402, and SEQ ID NO: 1559, respectively;
cxlvii. SEQ ID NO: 775, SEQ ID NO: 932, SEQ ID NO: 1089, SEQ ID NO: 1246, SEQ ID NO: 1403, and SEQ ID NO: 1560, respectively;
cxlviii. SEQ ID NO: 776, SEQ ID NO: 933, SEQ ID NO: 1090, SEQ ID NO: 1247, SEQ ID NO: 1404, and SEQ ID NO: 1561, respectively;
cxlix. SEQ ID NO: 777, SEQ ID NO: 934, SEQ ID NO: 1091, SEQ ID NO: 1248, SEQ ID NO: 1405, and SEQ ID NO: 1562, respectively;
cl. SEQ ID NO: 778, SEQ ID NO: 935, SEQ ID NO: 1092, SEQ ID NO: 1249, SEQ ID NO: 1406, and SEQ ID NO: 1563, respectively;
cli. SEQ ID NO: 779, SEQ ID NO: 936, SEQ ID NO: 1093, SEQ ID NO: 1250, SEQ ID NO: 1407, and SEQ ID NO: 1564, respectively;
clii. SEQ ID NO: 780, SEQ ID NO: 937, SEQ ID NO: 1094, SEQ ID NO: 1251, SEQ ID NO: 1408, and SEQ ID NO: 1565, respectively;
cliii. SEQ ID NO: 781, SEQ ID NO: 938, SEQ ID NO: 1095, SEQ ID NO: 1252, SEQ ID NO: 1409, and SEQ ID NO: 1566, respectively;
cliv. SEQ ID NO: 782, SEQ ID NO: 939, SEQ ID NO: 1096, SEQ ID NO: 1253, SEQ ID NO: 1410, and SEQ ID NO: 1567, respectively;
clv. SEQ ID NO: 783, SEQ ID NO: 940, SEQ ID NO: 1097, SEQ ID NO: 1254, SEQ ID NO: 1411, and SEQ ID NO: 1568, respectively;
clvi. SEQ ID NO: 784, SEQ ID NO: 941, SEQ ID NO: 1098, SEQ ID NO: 1255, SEQ ID NO: 1412, and SEQ ID NO: 1569, respectively; and
clvii. SEQ ID NO: 785, SEQ ID NO: 942, SEQ ID NO: 1099, SEQ ID NO: 1256, SEQ ID NO: 1413, and SEQ ID NO: 1570, respectively.

29. The molecule of any one of claims 1-28, wherein the antibody comprises a light chain variable region and a heavy chain variable region, wherein the light chain variable region and the heavy chain variable region comprise amino acid sequences selected from:
i. SEQ ID NO: 1 and SEQ ID NO: 158, respectively;
ii. SEQ ID NO: 2 and SEQ ID NO: 159, respectively;
iii. SEQ ID NO: 3 and SEQ ID NO: 160, respectively;
iv. SEQ ID NO: 4 and SEQ ID NO: 161, respectively;
v. SEQ ID NO: 5 and SEQ ID NO: 162, respectively;
vi. SEQ ID NO: 6 and SEQ ID NO: 163, respectively;
vii. SEQ ID NO: 7 and SEQ ID NO: 164, respectively;
viii. SEQ ID NO: 8 and SEQ ID NO: 165, respectively;
ix. SEQ ID NO: 9 and SEQ ID NO: 166, respectively;
x. SEQ ID NO: 10 and SEQ ID NO: 167, respectively;
xi. SEQ ID NO: 11 and SEQ ID NO: 168, respectively;
xii. SEQ ID NO: 12 and SEQ ID NO: 169, respectively;
xiii. SEQ ID NO: 13 and SEQ ID NO: 170, respectively;
xiv. SEQ ID NO: 14 and SEQ ID NO: 171, respectively;
xv. SEQ ID NO: 15 and SEQ ID NO: 172, respectively;
xvi. SEQ ID NO: 16 and SEQ ID NO: 173, respectively;
xvii. SEQ ID NO: 17 and SEQ ID NO: 174, respectively;
xviii. SEQ ID NO: 18 and SEQ ID NO: 175, respectively;
xix. SEQ ID NO: 19 and SEQ ID NO: 176, respectively;
xx. SEQ ID NO: 20 and SEQ ID NO: 177, respectively;
xxi. SEQ ID NO: 21 and SEQ ID NO: 178, respectively;
xxii. SEQ ID NO: 22 and SEQ ID NO: 179, respectively;
xxiii. SEQ ID NO: 23 and SEQ ID NO: 180, respectively;
xxiv. SEQ ID NO: 24 and SEQ ID NO: 181, respectively;
xxv. SEQ ID NO: 25 and SEQ ID NO: 182, respectively;
xxvi. SEQ ID NO: 26 and SEQ ID NO: 183, respectively;
xxvii. SEQ ID NO: 27 and SEQ ID NO: 184, respectively;
xxviii. SEQ ID NO: 28 and SEQ ID NO: 185, respectively;
xxix. SEQ ID NO: 29 and SEQ ID NO: 186, respectively;
xxx. SEQ ID NO: 30 and SEQ ID NO: 187, respectively;
xxxi. SEQ ID NO: 31 and SEQ ID NO: 188, respectively;
xxxii. SEQ ID NO: 32 and SEQ ID NO: 189, respectively;
xxxiii. SEQ ID NO: 33 and SEQ ID NO: 190, respectively;
xxxiv. SEQ ID NO: 34 and SEQ ID NO: 191, respectively;
xxxv. SEQ ID NO: 35 and SEQ ID NO: 192, respectively;
xxxvi. SEQ ID NO: 36 and SEQ ID NO: 193, respectively;
xxxvii. SEQ ID NO: 37 and SEQ ID NO: 194, respectively;
xxxviii. SEQ ID NO: 38 and SEQ ID NO: 195, respectively;
xxxix. SEQ ID NO: 39 and SEQ ID NO: 196, respectively;
xl. SEQ ID NO: 40 and SEQ ID NO: 197, respectively;
xli. SEQ ID NO: 41 and SEQ ID NO: 198, respectively;
xlii. SEQ ID NO: 42 and SEQ ID NO: 199, respectively;
xliii. SEQ ID NO: 43 and SEQ ID NO: 200, respectively;
xliv. SEQ ID NO: 44 and SEQ ID NO: 201, respectively;
xlv. SEQ ID NO: 45 and SEQ ID NO: 202, respectively;
xlvi. SEQ ID NO: 46 and SEQ ID NO: 203, respectively;
xlvii. SEQ ID NO: 47 and SEQ ID NO: 204, respectively;
xlviii. SEQ ID NO: 48 and SEQ ID NO: 205, respectively;
xlix. SEQ ID NO: 49 and SEQ ID NO: 206, respectively;
l. SEQ ID NO: 50 and SEQ ID NO: 207, respectively;
li. SEQ ID NO: 51 and SEQ ID NO: 208, respectively;
lii. SEQ ID NO: 52 and SEQ ID NO: 209, respectively;
liii. SEQ ID NO: 53 and SEQ ID NO: 210, respectively;
liv. SEQ ID NO: 54 and SEQ ID NO: 211, respectively;
lv. SEQ ID NO: 55 and SEQ ID NO: 212, respectively;
lvi. SEQ ID NO: 56 and SEQ ID NO: 213, respectively;
lvii. SEQ ID NO: 57 and SEQ ID NO: 214, respectively;
lviii. SEQ ID NO: 58 and SEQ ID NO: 215, respectively;
lix. SEQ ID NO: 59 and SEQ ID NO: 216, respectively;
lx. SEQ ID NO: 60 and SEQ ID NO: 217, respectively;
lxi. SEQ ID NO: 61 and SEQ ID NO: 218, respectively;
lxii. SEQ ID NO: 62 and SEQ ID NO: 219, respectively;
lxiii. SEQ ID NO: 63 and SEQ ID NO: 220, respectively;
lxiv. SEQ ID NO: 64 and SEQ ID NO: 221, respectively;
lxv. SEQ ID NO: 65 and SEQ ID NO: 222, respectively;
lxvi. SEQ ID NO: 66 and SEQ ID NO: 223, respectively;
lxvii. SEQ ID NO: 67 and SEQ ID NO: 224, respectively;
lxviii. SEQ ID NO: 68 and SEQ ID NO: 225, respectively;
lxix. SEQ ID NO: 69 and SEQ ID NO: 226, respectively;
lxx. SEQ ID NO: 70 and SEQ ID NO: 227, respectively;
lxxi. SEQ ID NO: 71 and SEQ ID NO: 228, respectively;
lxxii. SEQ ID NO: 72 and SEQ ID NO: 229, respectively;
lxxiii. SEQ ID NO: 73 and SEQ ID NO: 230, respectively;
lxxiv. SEQ ID NO: 74 and SEQ ID NO: 231, respectively;
lxxv. SEQ ID NO: 75 and SEQ ID NO: 232, respectively;
lxxvi. SEQ ID NO: 76 and SEQ ID NO: 233, respectively;
lxxvii. SEQ ID NO: 77 and SEQ ID NO: 234, respectively;
lxxviii. SEQ ID NO: 78 and SEQ ID NO: 235, respectively;
lxxix. SEQ ID NO: 79 and SEQ ID NO: 236, respectively;
lxxx. SEQ ID NO: 80 and SEQ ID NO: 237, respectively;
lxxxi. SEQ ID NO: 81 and SEQ ID NO: 238, respectively;
lxxxii. SEQ ID NO: 82 and SEQ ID NO: 239, respectively;
lxxxiii. SEQ ID NO: 83 and SEQ ID NO: 240, respectively;
lxxxiv. SEQ ID NO: 84 and SEQ ID NO: 241, respectively;
lxxxv. SEQ ID NO: 85 and SEQ ID NO: 242, respectively;
lxxxvi. SEQ ID NO: 86 and SEQ ID NO: 243, respectively;
lxxxvii. SEQ ID NO: 87 and SEQ ID NO: 244, respectively;
lxxxviii. SEQ ID NO: 88 and SEQ ID NO: 245, respectively;
lxxxix. SEQ ID NO: 89 and SEQ ID NO: 246, respectively;
xc. SEQ ID NO: 90 and SEQ ID NO: 247, respectively;
xci. SEQ ID NO: 91 and SEQ ID NO: 248, respectively;
xcii. SEQ ID NO: 92 and SEQ ID NO: 249, respectively;
xciii. SEQ ID NO: 93 and SEQ ID NO: 250, respectively;
xciv. SEQ ID NO: 94 and SEQ ID NO: 251, respectively;
xcv. SEQ ID NO: 95 and SEQ ID NO: 252, respectively;
xcvi. SEQ ID NO: 96 and SEQ ID NO: 253, respectively;
xcvii. SEQ ID NO: 97 and SEQ ID NO: 254, respectively;
xcviii. SEQ ID NO: 98 and SEQ ID NO: 255, respectively;
xcix. SEQ ID NO: 99 and SEQ ID NO: 256, respectively;
c. SEQ ID NO: 100 and SEQ ID NO: 257, respectively;
ci. SEQ ID NO: 101 and SEQ ID NO: 258, respectively;
cii. SEQ ID NO: 102 and SEQ ID NO: 259, respectively;
ciii. SEQ ID NO: 103 and SEQ ID NO: 260, respectively;
civ. SEQ ID NO: 104 and SEQ ID NO: 261, respectively;
cv. SEQ ID NO: 105 and SEQ ID NO: 262, respectively;
cvi. SEQ ID NO: 106 and SEQ ID NO: 263, respectively;
cvii. SEQ ID NO: 107 and SEQ ID NO: 264, respectively;
cviii. SEQ ID NO: 108 and SEQ ID NO: 265, respectively;
cix. SEQ ID NO: 109 and SEQ ID NO: 266, respectively;
cx. SEQ ID NO: 110 and SEQ ID NO: 267, respectively;
cxi. SEQ ID NO: 111 and SEQ ID NO: 268, respectively;
cxii. SEQ ID NO: 112 and SEQ ID NO: 269, respectively;
cxiii. SEQ ID NO: 113 and SEQ ID NO: 270, respectively;
cxiv. SEQ ID NO: 114 and SEQ ID NO: 271, respectively;
cxv. SEQ ID NO: 115 and SEQ ID NO: 272, respectively;
cxvi. SEQ ID NO: 116 and SEQ ID NO: 273, respectively;
cxvii. SEQ ID NO: 117 and SEQ ID NO: 274, respectively;
cxviii. SEQ ID NO: 118 and SEQ ID NO: 275, respectively;
cxix. SEQ ID NO: 119 and SEQ ID NO: 276, respectively;
cxx. SEQ ID NO: 120 and SEQ ID NO: 277, respectively;
cxxi. SEQ ID NO: 121 and SEQ ID NO: 278, respectively;
cxxii. SEQ ID NO: 122 and SEQ ID NO: 279, respectively;
cxxiii. SEQ ID NO: 123 and SEQ ID NO: 280, respectively;
cxxiv. SEQ ID NO: 124 and SEQ ID NO: 281, respectively;
cxxv. SEQ ID NO: 125 and SEQ ID NO: 282, respectively;
cxxvi. SEQ ID NO: 126 and SEQ ID NO: 283, respectively;
cxxvii. SEQ ID NO: 127 and SEQ ID NO: 284, respectively;
cxxviii. SEQ ID NO: 128 and SEQ ID NO: 285, respectively;
cxxix. SEQ ID NO: 129 and SEQ ID NO: 286, respectively;
cxxx. SEQ ID NO: 130 and SEQ ID NO: 287, respectively;
cxxxi. SEQ ID NO: 131 and SEQ ID NO: 288, respectively;
cxxxii. SEQ ID NO: 132 and SEQ ID NO: 289, respectively;
cxxxiii. SEQ ID NO: 133 and SEQ ID NO: 290, respectively;
cxxxiv. SEQ ID NO: 134 and SEQ ID NO: 291, respectively;
cxxxv. SEQ ID NO: 135 and SEQ ID NO: 292, respectively;
cxxxvi. SEQ ID NO: 136 and SEQ ID NO: 293, respectively;
cxxxvii. SEQ ID NO: 137 and SEQ ID NO: 294, respectively;
cxxxviii. SEQ ID NO: 138 and SEQ ID NO: 295, respectively;
cxxxix. SEQ ID NO: 139 and SEQ ID NO: 296, respectively;
cxl. SEQ ID NO: 140 and SEQ ID NO: 297, respectively;
cxli. SEQ ID NO: 141 and SEQ ID NO: 298, respectively;
cxlii. SEQ ID NO: 142 and SEQ ID NO: 299, respectively;
cxliii. SEQ ID NO: 143 and SEQ ID NO: 300, respectively;
cxliv. SEQ ID NO: 144 and SEQ ID NO: 301, respectively;
cxlv. SEQ ID NO: 145 and SEQ ID NO: 302, respectively;
cxlvi. SEQ ID NO: 146 and SEQ ID NO: 303, respectively;
cxlvii. SEQ ID NO: 147 and SEQ ID NO: 304, respectively;
cxlviii. SEQ ID NO: 148 and SEQ ID NO: 305, respectively;
cxlix. SEQ ID NO: 149 and SEQ ID NO: 306, respectively;
cl. SEQ ID NO: 150 and SEQ ID NO: 307, respectively;
cli. SEQ ID NO: 151 and SEQ ID NO: 308, respectively;
clii. SEQ ID NO: 152 and SEQ ID NO: 309, respectively;
cliii. SEQ ID NO: 153 and SEQ ID NO: 310, respectively;
cliv. SEQ ID NO: 154 and SEQ ID NO: 311, respectively;
clv. SEQ ID NO: 155 and SEQ ID NO: 312, respectively;
clvi. SEQ ID NO: 156 and SEQ ID NO: 313, respectively; and
clvii. SEQ ID NO: 157 and SEQ ID NO: 314, respectively.

30. The molecule of any one of claims 1-29, wherein the antibody comprises a light chain and a heavy chain, wherein the light chain and heavy chain comprise amino acid sequences selected from:
i. SEQ ID NO: 315 and SEQ ID NO: 472, respectively;
ii. SEQ ID NO: 316 and SEQ ID NO: 473, respectively;
iii. SEQ ID NO: 317 and SEQ ID NO: 474, respectively;
iv. SEQ ID NO: 318 and SEQ ID NO: 475, respectively;
v. SEQ ID NO: 319 and SEQ ID NO: 476, respectively;
vi. SEQ ID NO: 320 and SEQ ID NO: 477, respectively;
vii. SEQ ID NO: 321 and SEQ ID NO: 478, respectively;
viii. SEQ ID NO: 322 and SEQ ID NO: 479, respectively;
ix. SEQ ID NO: 323 and SEQ ID NO: 480, respectively;
x. SEQ ID NO: 324 and SEQ ID NO: 481, respectively;
xi. SEQ ID NO: 325 and SEQ ID NO: 482, respectively;
xii. SEQ ID NO: 326 and SEQ ID NO: 483, respectively;
xiii. SEQ ID NO: 327 and SEQ ID NO: 484, respectively;
xiv. SEQ ID NO: 328 and SEQ ID NO: 485, respectively;
xv. SEQ ID NO: 329 and SEQ ID NO: 486, respectively;
xvi. SEQ ID NO: 330 and SEQ ID NO: 487, respectively;
xvii. SEQ ID NO: 331 and SEQ ID NO: 488, respectively;
xviii. SEQ ID NO: 332 and SEQ ID NO: 489, respectively;
xix. SEQ ID NO: 333 and SEQ ID NO: 490, respectively;
xx. SEQ ID NO: 334 and SEQ ID NO: 491, respectively;
xxi. SEQ ID NO: 335 and SEQ ID NO: 492, respectively;
xxii. SEQ ID NO: 336 and SEQ ID NO: 493, respectively;
xxiii. SEQ ID NO: 337 and SEQ ID NO: 494, respectively;
xxiv. SEQ ID NO: 338 and SEQ ID NO: 495, respectively;
xxv. SEQ ID NO: 339 and SEQ ID NO: 496, respectively;
xxvi. SEQ ID NO: 340 and SEQ ID NO: 497, respectively;
xxvii. SEQ ID NO: 341 and SEQ ID NO: 498, respectively;
xxviii. SEQ ID NO: 342 and SEQ ID NO: 499, respectively;
xxix. SEQ ID NO: 343 and SEQ ID NO: 500, respectively;
xxx. SEQ ID NO: 344 and SEQ ID NO: 501, respectively;
xxxi. SEQ ID NO: 345 and SEQ ID NO: 502, respectively;
xxxii. SEQ ID NO: 346 and SEQ ID NO: 503, respectively;
xxxiii. SEQ ID NO: 347 and SEQ ID NO: 504, respectively;
xxxiv. SEQ ID NO: 348 and SEQ ID NO: 505, respectively;
xxxv. SEQ ID NO: 349 and SEQ ID NO: 506, respectively;
xxxvi. SEQ ID NO: 350 and SEQ ID NO: 507, respectively;
xxxvii. SEQ ID NO: 351 and SEQ ID NO: 508, respectively;
xxxviii. SEQ ID NO: 352 and SEQ ID NO: 509, respectively;
xxxix. SEQ ID NO: 353 and SEQ ID NO: 510, respectively;
xl. SEQ ID NO: 354 and SEQ ID NO: 511, respectively;
xli. SEQ ID NO: 355 and SEQ ID NO: 512, respectively;
xlii. SEQ ID NO: 356 and SEQ ID NO: 513, respectively;
xliii. SEQ ID NO: 357 and SEQ ID NO: 514, respectively;
xliv. SEQ ID NO: 358 and SEQ ID NO: 515, respectively;
xlv. SEQ ID NO: 359 and SEQ ID NO: 516, respectively;
xlvi. SEQ ID NO: 360 and SEQ ID NO: 517, respectively;
xlvii. SEQ ID NO: 361 and SEQ ID NO: 518, respectively;
xlviii. SEQ ID NO: 362 and SEQ ID NO: 519, respectively;
xlix. SEQ ID NO: 363 and SEQ ID NO: 520, respectively;
l. SEQ ID NO: 364 and SEQ ID NO: 521, respectively;
li. SEQ ID NO: 365 and SEQ ID NO: 522, respectively;
lii. SEQ ID NO: 366 and SEQ ID NO: 523, respectively;
liii. SEQ ID NO: 367 and SEQ ID NO: 524, respectively;
liv. SEQ ID NO: 368 and SEQ ID NO: 525, respectively;
lv. SEQ ID NO: 369 and SEQ ID NO: 526, respectively;
lvi. SEQ ID NO: 370 and SEQ ID NO: 527, respectively;
lvii. SEQ ID NO: 371 and SEQ ID NO: 528, respectively;
lviii. SEQ ID NO: 372 and SEQ ID NO: 529, respectively;
lix. SEQ ID NO: 373 and SEQ ID NO: 530, respectively;
lx. SEQ ID NO: 374 and SEQ ID NO: 531, respectively;
lxi. SEQ ID NO: 375 and SEQ ID NO: 532, respectively;
lxii. SEQ ID NO: 376 and SEQ ID NO: 533, respectively;
lxiii. SEQ ID NO: 377 and SEQ ID NO: 534, respectively;
lxiv. SEQ ID NO: 378 and SEQ ID NO: 535, respectively;
lxv. SEQ ID NO: 379 and SEQ ID NO: 536, respectively;
lxvi. SEQ ID NO: 380 and SEQ ID NO: 537, respectively;
lxvii. SEQ ID NO: 381 and SEQ ID NO: 538, respectively;
lxviii. SEQ ID NO: 382 and SEQ ID NO: 539, respectively;
lxix. SEQ ID NO: 383 and SEQ ID NO: 540, respectively;
lxx. SEQ ID NO: 384 and SEQ ID NO: 541, respectively;
lxxi. SEQ ID NO: 385 and SEQ ID NO: 542, respectively;
lxxii. SEQ ID NO: 386 and SEQ ID NO: 543, respectively;
lxxiii. SEQ ID NO: 387 and SEQ ID NO: 544, respectively;
lxxiv. SEQ ID NO: 388 and SEQ ID NO: 545, respectively;
lxxv. SEQ ID NO: 389 and SEQ ID NO: 546, respectively;
lxxvi. SEQ ID NO: 390 and SEQ ID NO: 547, respectively;
lxxvii. SEQ ID NO: 391 and SEQ ID NO: 548, respectively;
lxxviii. SEQ ID NO: 392 and SEQ ID NO: 549, respectively;
lxxix. SEQ ID NO: 393 and SEQ ID NO: 550, respectively;
lxxx. SEQ ID NO: 394 and SEQ ID NO: 551, respectively;
lxxxi. SEQ ID NO: 395 and SEQ ID NO: 552, respectively;
lxxxii. SEQ ID NO: 396 and SEQ ID NO: 553, respectively;
lxxxiii. SEQ ID NO: 397 and SEQ ID NO: 554, respectively;
lxxxiv. SEQ ID NO: 398 and SEQ ID NO: 555, respectively;
lxxxv. SEQ ID NO: 399 and SEQ ID NO: 556, respectively;
lxxxvi. SEQ ID NO: 400 and SEQ ID NO: 557, respectively;
lxxxvii. SEQ ID NO: 401 and SEQ ID NO: 558, respectively;
lxxxviii. SEQ ID NO: 402 and SEQ ID NO: 559, respectively;
lxxxix. SEQ ID NO: 403 and SEQ ID NO: 560, respectively;
xc. SEQ ID NO: 404 and SEQ ID NO: 561, respectively;
xci. SEQ ID NO: 405 and SEQ ID NO: 562, respectively;
xcii. SEQ ID NO: 406 and SEQ ID NO: 563, respectively;
xciii. SEQ ID NO: 407 and SEQ ID NO: 564, respectively;
xciv. SEQ ID NO: 408 and SEQ ID NO: 565, respectively;
xcv. SEQ ID NO: 409 and SEQ ID NO: 566, respectively;
xcvi. SEQ ID NO: 410 and SEQ ID NO: 567, respectively;
xcvii. SEQ ID NO: 411 and SEQ ID NO: 568, respectively;
xcviii. SEQ ID NO: 412 and SEQ ID NO: 569, respectively;
xcix. SEQ ID NO: 413 and SEQ ID NO: 570, respectively;
c. SEQ ID NO: 414 and SEQ ID NO: 571, respectively;
ci. SEQ ID NO: 415 and SEQ ID NO: 572, respectively;
cii. SEQ ID NO: 416 and SEQ ID NO: 573, respectively;
ciii. SEQ ID NO: 417 and SEQ ID NO: 574, respectively;
civ. SEQ ID NO: 418 and SEQ ID NO: 575, respectively;
cv. SEQ ID NO: 419 and SEQ ID NO: 576, respectively;
cvi. SEQ ID NO: 420 and SEQ ID NO: 577, respectively;
cvii. SEQ ID NO: 421 and SEQ ID NO: 578, respectively;
cviii. SEQ ID NO: 422 and SEQ ID NO: 579, respectively;
cix. SEQ ID NO: 423 and SEQ ID NO: 580, respectively;
cx. SEQ ID NO: 424 and SEQ ID NO: 581, respectively;
cxi. SEQ ID NO: 425 and SEQ ID NO: 582, respectively;
cxii. SEQ ID NO: 426 and SEQ ID NO: 583, respectively;
cxiii. SEQ ID NO: 427 and SEQ ID NO: 584, respectively;
cxiv. SEQ ID NO: 428 and SEQ ID NO: 585, respectively;
cxv. SEQ ID NO: 429 and SEQ ID NO: 586, respectively;
cxvi. SEQ ID NO: 430 and SEQ ID NO: 587, respectively;
cxvii. SEQ ID NO: 431 and SEQ ID NO: 588, respectively;
cxviii. SEQ ID NO: 432 and SEQ ID NO: 589, respectively;
cxix. SEQ ID NO: 433 and SEQ ID NO: 590, respectively;
cxx. SEQ ID NO: 434 and SEQ ID NO: 591, respectively;
cxxi. SEQ ID NO: 435 and SEQ ID NO: 592, respectively;
cxxii. SEQ ID NO: 436 and SEQ ID NO: 593, respectively;
cxxiii. SEQ ID NO: 437 and SEQ ID NO: 594, respectively;
cxxiv. SEQ ID NO: 438 and SEQ ID NO: 595, respectively;
cxxv. SEQ ID NO: 439 and SEQ ID NO: 596, respectively;
cxxvi. SEQ ID NO: 440 and SEQ ID NO: 597, respectively;
cxxvii. SEQ ID NO: 441 and SEQ ID NO: 598, respectively;
cxxviii. SEQ ID NO: 442 and SEQ ID NO: 599, respectively;
cxxix. SEQ ID NO: 443 and SEQ ID NO: 600, respectively;
cxxx. SEQ ID NO: 444 and SEQ ID NO: 601, respectively;
cxxxi. SEQ ID NO: 445 and SEQ ID NO: 602, respectively;
cxxxii. SEQ ID NO: 446 and SEQ ID NO: 603, respectively;
cxxxiii. SEQ ID NO: 447 and SEQ ID NO: 604, respectively;
cxxxiv. SEQ ID NO: 448 and SEQ ID NO: 605, respectively;
cxxxv. SEQ ID NO: 449 and SEQ ID NO: 606, respectively;
cxxxvi. SEQ ID NO: 450 and SEQ ID NO: 607, respectively;
cxxxvii. SEQ ID NO: 451 and SEQ ID NO: 608, respectively;
cxxxviii. SEQ ID NO: 452 and SEQ ID NO: 609, respectively;
cxxxix. SEQ ID NO: 453 and SEQ ID NO: 610, respectively;
cxl. SEQ ID NO: 454 and SEQ ID NO: 611, respectively;
cxli. SEQ ID NO: 455 and SEQ ID NO: 612, respectively;
cxlii. SEQ ID NO: 456 and SEQ ID NO: 613, respectively;
cxliii. SEQ ID NO: 457 and SEQ ID NO: 614, respectively;
cxliv. SEQ ID NO: 458 and SEQ ID NO: 615, respectively;
cxlv. SEQ ID NO: 459 and SEQ ID NO: 616, respectively;
cxlvi. SEQ ID NO: 460 and SEQ ID NO: 617, respectively;
cxlvii. SEQ ID NO: 461 and SEQ ID NO: 618, respectively;
cxlviii. SEQ ID NO: 462 and SEQ ID NO: 619, respectively;
cxlix. SEQ ID NO: 463 and SEQ ID NO: 620, respectively;
cl. SEQ ID NO: 464 and SEQ ID NO: 621, respectively;
cli. SEQ ID NO: 465 and SEQ ID NO: 622, respectively;
clii. SEQ ID NO: 466 and SEQ ID NO: 623, respectively;
cliii. SEQ ID NO: 467 and SEQ ID NO: 624, respectively;
cliv. SEQ ID NO: 468 and SEQ ID NO: 625, respectively;
clv. SEQ ID NO: 469 and SEQ ID NO: 626, respectively;
clvi. SEQ ID NO: 470 and SEQ ID NO: 627, respectively; and
clvii. SEQ ID NO: 471 and SEQ ID NO: 628, respectively,
wherein the antibody comprises one or more cysteine amino acid substitution(s) at one or more position(s) selected from 88 of the light chain, 384 of the heavy chain, or 487 of the heavy chain, according to AHo numbering.

31. A molecule comprising:
a first polypeptide and a second polypeptide that agonize a glucagon receptor (“GCGR”), wherein each of the first polypeptide and the second polypeptide comprise an amino acid sequence independently selected from SEQ ID NOs: 1587-1627, 1747-1749, 1751-1792, 1794-1798, 1801-1830, 1833-1840, and 1859-1862;
a first linker polypeptide and a second linker polypeptide, wherein each of the first linker polypeptide and the second linker polypeptide comprise an amino acid sequence selected from SEQ ID NOs: 1628-1683, 1739-1746, and 1841-1852; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
an ε-amino group of a lysine residue at position 21, position 24, position 28, or position 31 of the first polypeptide is covalently linked to a C-terminus of the first linker polypeptide;
an N-terminus of the first linker polypeptide is conjugated to a cysteine residue at position 275 of the first heavy chain of the antibody;
an ε-amino group of a lysine residue at position 21, position 24, position 28, or position 31 of the second polypeptide is covalently linked to a C-terminus of the second linker polypeptide; and
an N-terminus of the second linker polypeptide is conjugated to a cysteine residue at position 275 of the second heavy chain of the antibody.

32. A molecule comprising:
a first polypeptide and a second polypeptide that agonize a glucagon receptor (“GCGR”), wherein each of the first polypeptide and the second polypeptide comprise an amino acid sequence independently selected from SEQ ID NOs: 1587-1627, 1747-1840, and 1859-1862;
a first linker polypeptide and a second linker polypeptide, wherein each of the first linker polypeptide and the second linker polypeptide comprise an amino acid sequence selected from SEQ ID NOs: 1628-1683, 1739-1746, and 1841-1852; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
a C-terminal amino acid residue of the first polypeptide is covalently linked to an N-terminal amino acid residue of the first linker polypeptide;
a C-terminal amino acid residue of the first linker polypeptide is conjugated to a cysteine residue at position 275 of the first heavy chain of the antibody;
a C-terminal amino acid residue of the second polypeptide is covalently linked to an N-terminal amino acid residue of the second linker polypeptide; and
a C-terminal amino acid residue of the second linker polypeptide is conjugated to a cysteine residue at position 275 of the second heavy chain of the antibody.

33. A molecule comprising:
a first polypeptide and a second polypeptide that agonize a glucagon receptor (“GCGR”), wherein the first polypeptide and the second polypeptide each comprise the amino acid sequence of SEQ ID NO: 1615;
a first linker polypeptide and a second linker polypeptide, wherein the first linker polypeptide and the second linker polypeptide each comprise the amino acid sequence of SEQ ID NO: 1629; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
an ε-amino group of a lysine residue at position 28 of the first polypeptide is covalently linked to a C-terminus of the first linker polypeptide;
an N-terminus of the first linker polypeptide is conjugated to a cysteine residue at position 275 of the first heavy chain of the antibody;
an ε-amino group of a lysine residue at position 28 of the second polypeptide is covalently linked to a C-terminus of the second linker polypeptide; and
an N-terminus of the second linker polypeptide is conjugated to a cysteine residue at position 275 of the second heavy chain of the antibody.

34. A molecule comprising:
a first polypeptide and a second polypeptide that agonize a glucagon receptor (“GCGR”), wherein the first polypeptide and the second polypeptide each comprise the amino acid sequence of SEQ ID NO: 1626;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
an ε-amino group of a lysine residue at position 28 of the first polypeptide is covalently linked to a C-terminus of the first linker polypeptide;
an N-terminus of the first linker polypeptide is conjugated to a cysteine residue at position 275 of the first heavy chain of the antibody;
an ε-amino group of a lysine residue at position 28 of the second polypeptide is covalently linked to a C-terminus of the second linker polypeptide; and
an N-terminus of the second linker polypeptide is conjugated to a cysteine residue at position 275 of the second heavy chain of the antibody.

35. A molecule comprising:
a first polypeptide and a second polypeptide that agonize a glucagon receptor (“GCGR”), wherein the first polypeptide and the second polypeptide each comprise the amino acid sequence of SEQ ID NO: 1592;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
an ε-amino group of a lysine residue at position 24 of the first polypeptide is covalently linked to a C-terminus of the first linker polypeptide;
an N-terminus of the first linker polypeptide is conjugated to a cysteine residue at position 275 of the first heavy chain of the antibody;
an ε-amino group of a lysine residue at position 24 of the second polypeptide is covalently linked to a C-terminus of the second linker polypeptide; and
an N-terminus of the second linker polypeptide is conjugated to a cysteine residue at position 275 of the second heavy chain of the antibody.

36. A molecule comprising:
a first polypeptide and a second polypeptide that agonize a glucagon receptor (“GCGR”), wherein the first polypeptide and the second polypeptide each comprise the amino acid sequence of SEQ ID NO: 1626;
a first linker polypeptide and a second linker polypeptide, wherein the first linker polypeptide and the second linker polypeptide each comprise the amino acid sequence of SEQ ID NO: 1629; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
an ε-amino group of a lysine residue at position 28 of the first polypeptide is covalently linked to a C-terminus of the first linker polypeptide;
an N-terminus of the first linker polypeptide is conjugated to a cysteine residue at position 275 of the first heavy chain of the antibody;
an ε-amino group of a lysine residue at position 28 of the second polypeptide is covalently linked to a C-terminus of the second linker polypeptide; and
an N-terminus of the second linker polypeptide is conjugated to a cysteine residue at position 275 of the second heavy chain of the antibody.

37. A molecule comprising:
a first polypeptide and a second polypeptide that agonize a glucagon receptor (“GCGR”), wherein the first polypeptide and the second polypeptide each comprise the amino acid sequence of SEQ ID NO: 1587;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
an ε-amino group of a lysine residue at position 28 of the first polypeptide is covalently linked to a C-terminus of the first linker polypeptide;
an N-terminus of the first linker polypeptide is conjugated to a cysteine residue at position 275 of the first heavy chain of the antibody;
an ε-amino group of a lysine residue at position 28 of the second polypeptide is covalently linked to a C-terminus of the second linker polypeptide; and
an N-terminus of the second linker polypeptide is conjugated to a cysteine residue at position 275 of the second heavy chain of the antibody.

38. A molecule comprising:
a first polypeptide and a second polypeptide that agonize a glucagon receptor (“GCGR”), wherein the first polypeptide and the second polypeptide each comprise the amino acid sequence of SEQ ID NO: 1587;
a first linker polypeptide and a second linker polypeptide, wherein the first linker polypeptide and the second linker polypeptide each comprise the amino acid sequence of SEQ ID NO: 1630; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
an ε-amino group of a lysine residue at position 28 of the first polypeptide is covalently linked to a C-terminus of the first linker polypeptide;
an N-terminus of the first linker polypeptide is conjugated to a cysteine residue at position 275 of the first heavy chain of the antibody;
an ε-amino group of a lysine residue at position 28 of the second polypeptide is covalently linked to a C-terminus of the second linker polypeptide; and
an N-terminus of the second linker polypeptide is conjugated to a cysteine residue at position 275 of the second heavy chain of the antibody.

39. A molecule comprising:
a first polypeptide and a second polypeptide that agonize a glucagon receptor (“GCGR”), wherein the first polypeptide and the second polypeptide each comprise the amino acid sequence of SEQ ID NO: 1822;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
an ε-amino group of a lysine residue at position 24 of the first polypeptide is covalently linked to a C-terminus of the first linker polypeptide;
an N-terminus of the first linker polypeptide is conjugated to a cysteine residue at position 275 of the first heavy chain of the antibody;
an ε-amino group of a lysine residue at position 24 of the second polypeptide is covalently linked to a C-terminus of the second linker polypeptide; and
an N-terminus of the second linker polypeptide is conjugated to a cysteine residue at position 275 of the second heavy chain of the antibody.

40. A molecule comprising:
a first polypeptide and a second polypeptide that agonize a glucagon receptor (“GCGR”), wherein the first polypeptide and the second polypeptide each comprise the amino acid sequence of SEQ ID NO: 1825;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
an ε-amino group of a lysine residue at position 28 of the first polypeptide is covalently linked to a C-terminus of the first linker polypeptide;
an N-terminus of the first linker polypeptide is conjugated to a cysteine residue at position 275 of the first heavy chain of the antibody;
an ε-amino group of a lysine residue at position 28 of the second polypeptide is covalently linked to a C-terminus of the second linker polypeptide; and
an N-terminus of the second linker polypeptide is conjugated to a cysteine residue at position 275 of the second heavy chain of the antibody.

41. A molecule comprising:
a first polypeptide and a second polypeptide that agonize a glucagon receptor (“GCGR”), wherein the first polypeptide and the second polypeptide each comprise the amino acid sequence of SEQ ID NO: 1826;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
an ε-amino group of a lysine residue at position 28 of the first polypeptide is covalently linked to a C-terminus of the first linker polypeptide;
an N-terminus of the first linker polypeptide is conjugated to a cysteine residue at position 275 of the first heavy chain of the antibody;
an ε-amino group of a lysine residue at position 28 of the second polypeptide is covalently linked to a C-terminus of the second linker polypeptide; and
an N-terminus of the second linker polypeptide is conjugated to a cysteine residue at position 275 of the second heavy chain of the antibody.

42. A molecule comprising:
a first polypeptide and a second polypeptide that agonize a glucagon receptor (“GCGR”), wherein the first polypeptide and the second polypeptide each comprise the amino acid sequence of SEQ ID NO: 1818;
a first linker polypeptide and a second linker polypeptide, wherein the first linker polypeptide and the second linker polypeptide each comprise the amino acid sequence of SEQ ID NO: 1628; and
an antibody that specifically binds to a glucose-dependent insulinotropic polypeptide receptor (“GIPR”), wherein the antibody comprises a first light chain and a second light chain, wherein the first light chain and the second light chain each comprise the amino acid sequence of SEQ ID NO: 388, and a first heavy chain and a second heavy chain, wherein the first heavy chain and the second heavy chain each comprise the amino acid sequence of SEQ ID NO: 1571,
wherein:
an ε-amino group of a lysine residue at position 28 of the first polypeptide is covalently linked to a C-terminus of the first linker polypeptide;
an N-terminus of the first linker polypeptide is conjugated to a cysteine residue at position 275 of the first heavy chain of the antibody;
an ε-amino group of a lysine residue at position 28 of the second polypeptide is covalently linked to a C-terminus of the second linker polypeptide; and
an N-terminus of the second linker polypeptide is conjugated to a cysteine residue at position 275 of the second heavy chain of the antibody.

43. A molecule comprising:
a polypeptide that agonizes a glucagon receptor (“GCGR”), wherein the polypeptide comprises the amino acid sequence of any one of SEQ ID NOs: 1587-1627, 1747-1840, 1859-1862, or 1879-1881; and
a half-life extending domain.

44. The molecule of claim 43, wherein the half-life extending domain is an Fc-containing polypeptide.

45. A pharmaceutical composition comprising:
a molecule of any one of claims 1-44; and
a pharmaceutically acceptable excipient.

46. A method of reducing body weight and/or food intake in a subject in need thereof, the method comprising administering a molecule of any one of claims 1-44 or a pharmaceutical composition of claim 45 to the subject.

47. A molecule of any one of claims 1-44 or a pharmaceutical composition of claim 45 for use in a method of reducing body weight and/or food intake in a subject in need thereof.

48. A method of reducing body weight and/or food intake in a subject in need thereof, the method comprising administering a molecule of any one of claims 1-44 or a pharmaceutical composition of claim 45 to the subject in combination with a GLP-1 agonist.

49. A molecule of any one of claims 1-44 or a pharmaceutical composition of claim 45 for use in a method of reducing body weight and/or food intake in a subject in need thereof, wherein the method comprises administering the molecule or pharmaceutical composition to the subject in combination with a GLP-1 agonist.

50. The method of claim 48 or the molecule or pharmaceutical composition for use of claim 49, wherein the subject is overweight or obese and the GLP-1 agonist is semaglutide.
